(19) United States
(12) Patent Application Publication
(10) Pub. No.: US 2025/0340551 A1
(43) Pub. Date: Nov. 6, 2025

(54) SMALL MOLECULE MODULATORS OF STAT6

(71) Applicant: Gilead Sciences, Inc., Foster City, CA (US)

(72) Inventors: Pablo Martín-Gago, Frederiksberg (DK); Nadia Nasser Petersen, Ballerup (DK); Bjarne Nørremark, Stenløse (DK); Sebastian Clementson, Malmö (SE); Morten Dahl Sørensen, Hørsholm (DK); Shaoquan Lin, Ballerup (DK); Kevin Neil Dack, Kongens Lyngby (DK); Morten Jørgensen, Herlev (DK); Marcel John de Groot, Værløse (DK); Mia Noerreskov Burhardt, Valby (DK)

(21) Appl. No.: 19/091,644

(22) Filed: Mar. 26, 2025

(30) Foreign Application Priority Data
Mar. 27, 2024 (EP) EP24167010.8

(51) Int. Cl. C07D 471/04; A61K 31/4545; A61K 31/496; A61K 31/497; A61K 31/501; A61K 31/506; A61K 31/53; C07B 59/00

(52) U.S. Cl. CPC C07D 471/04; A61K 31/4545; A61K 31/496; A61K 31/497; A61K 31/501; A61K 31/506; A61K 31/53; C07B 59/002

(57) ABSTRACT

The present disclosure relates to a compound according to formula (I)

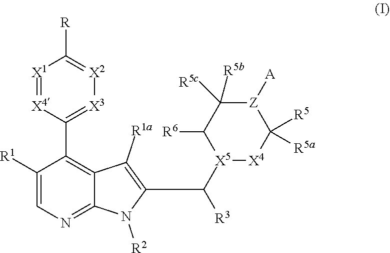

and pharmaceutically acceptable salts, hydrates, or solvates thereof. The disclosure further relates to said compounds for use in therapy, to pharmaceutical compositions comprising said compounds, to methods of treating diseases, e.g. dermal diseases, with said compounds, and to the use of said compounds in the manufacture of medicaments.

### CROSS-REFERENCE TO RELATED APPLICATIONS

This application claims priority to European Patent Application Number EP24167010.8, filed Mar. 27, 2024, the contents of which is hereby incorporated by reference in its entirety.

### FIELD

Provided herein are novel azaindoles and derivatives thereof and their use in therapy, as well as pharmaceutical compositions comprising said compounds.

### BACKGROUND

The disclosure relates generally to methods and compounds, and pharmaceutically acceptable salts thereof, for modulating a Signal transducer and activator of transcript 6 (STAT6) protein activity and treating STAT6 associated diseases. The following description set forth exemplary methods, parameters and the like. It should be recognized, however, that such description is not intended as a limitation on the scope of the present disclosure but instead provided as a description of exemplary embodiments.

Compound of the present disclosure include small molecule modulators of STAT6.

The signal Transducer and Activator of Transcription 6 (STAT6) belongs to a family of transcription factors (STAT1, STAT2, STAT3, STAT4, STAT5a, STAT5b and STAT6) which may be structurally and/or functionally related, and which may be involved in mediating signaling from multiple cytokine and/or growth factor receptors.

Without being bound by theory, STAT6 can selectively mediate signaling from IL-4 and IL-13 via the IL-4Ra subunit complexing with either the common gamma chain (γc) to form the type I receptor or with the IL-13Rα1 subunit to form a type II receptor. When IL-4 or IL-13 activates the receptor complex, the Janus Kinases (Jak) associated with the cytoplasmic tail of IL-4Ra can be activated and phosphorylate tyrosine residues on the intracellular part of the receptor. This phosphorylation can generate docking site(s) for STAT6, which can bind to the phosphorylated receptor via its Src homology-2 (SH2) domain. This may allow Jak kinases to phosphorylate tyrosine (Y)-641 on STAT6, potentially leading to activation. Activated STAT6 may form a homodimer and relocate to the nucleus and activate gene transcription. The genes transcribed by activated STAT6 can be cell specific and could in general induce Th2 immune responses (Walford and Taylor 2013: STAT6 and lung inflammation. JAK-STAT 2:4, e25301; October/November/December 2013; © 2013 Landes Bioscience).

STAT6 is expressed in numerous cell types including epithelial cells, fibroblasts and immune cells.

Without being bound by theory, inhibition of STAT6 activity can inhibit the IL-4 and IL-13 mediated effects in cells, including the differentiation of T-cells into Th2 cells and B-cell class shift into IgE and IgG1 producing cells (Walford). In epidermal keratinocytes, a STAT6 inhibitor could inhibit the secretion of pro-inflammatory chemokines and revert the cytokine-induced inhibition of barrier function proteins such as filaggrin (Tollenaire et al 2017: Skin Barrier and Inflammation Genes Associated with Atopic Dermatitis are Regulated by Interleukin-13 and Modulated by Tralokinumab In vitro. Acta Derm Venereol 2021; 101: adv00447).

Antibodies targeting Th2 immune responses such as the IL-4Ra (dupilumab) or IL-13 (tralokinumab, lebrikizumab) have shown efficacy in a number of Th2-driven diseases. Targeting STAT6 with a small molecule inhibitor allows for targeting the same pathway by an oral or dermal administration route and may have efficacy in diseases where Dupilumab has shown effect. A compound antagonizing STAT6 could, therefore, have utility in treating conditions characterized by Th2-mediated inflammation such as atopic dermatitis, prurigo nodularis, Bullous phemphigoid, asthma, chronic rhinosinusitis with nasal polyposis, urticaria (such as chronic spontaneous urticaria), rhinitis, eosinophilic esophagitis, food allergy diffuse cutaneous systemic sclerosis, alopecia areata and/or COPD.

STAT6 is also involved in differentiation and activity of M2 macrophages, including the tumor-associated macrophages (TAMs) in solid tumors. TAMs protect the tumor from immune attack by inducing a pro-tumor immunosuppressive environment. TAMs may inhibit T-cell proliferation, block migration of CD8 T-cells into the tumor and recruit Tregs into the tumor microenvironment (Karpathiou et al 2021: STAT6: A review of a signaling pathway implicated in various diseases with a special emphasis in its usefulness in pathology. Pathology—Research and Practice 223 (2021) 153477).

In addition, IL-13 may act as a growth factor for some tumors and for some tumors gain-of-function mutations in STAT6 have been described as oncogenes (Karpathiou et al 2021: STAT6: A review of a signaling pathway implicated in various diseases with a special emphasis in its usefulness in pathology. Pathology—Research and Practice 223 (2021) 153477).

Together these data suggests that a STAT6 inhibitor may treat different cancers such as lymphomas, non-small cell lung cancer and solid fibrous cancers either as a stand-alone treatment or in combination with other anticancer drugs such as check point inhibitors.

Although various antibodies against IL-4R or IL-13 are approved for medical use, there are currently no approved, orally available modulators of STAT6.

Therefore, there remains a continuous need to develop small molecule modulators of STAT-6, particularly small molecules suitable for oral administration.

In addition, some patients may be treated by topical application of small molecule modulators of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems.

### SUMMARY

The inventors have surprisingly found that novel compounds of the present disclosure exhibit modulating effects on the STAT-6 signalling pathway.

Compounds in embodiments of the present disclosure may be beneficial in preventing, treating, or ameliorating a variety of disease which involve up-regulation or de-regulation of STAT-6.

Compounds in embodiments of the present disclosure have advantageous properties such as high metabolic stability, membrane permeability and/or solubility that make them particularly suitable for oral administration.

Moreover, some patients may be treated by topical application of degraders of STAT-6. This can be particularly suitable, for example, for patients with skin lesions that are readily accessible and limited to areas on the body surface. Topical treatment may also be prescribed for certain patients who could benefit from avoiding systemic modulation of the STAT-6 pathway, for example when undergoing treatment for infections or gastrointestinal problems. Thus, some aspects of the present disclosure relate to methods for the topical application of the compounds and salts thereof as described herein.

Accordingly, in some embodiments, the present disclosure provides a compound according to formula (I):

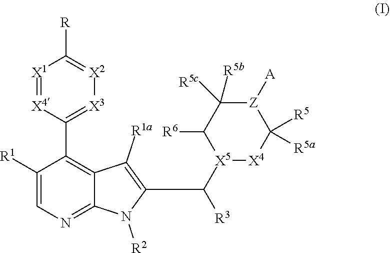

A is selected from,

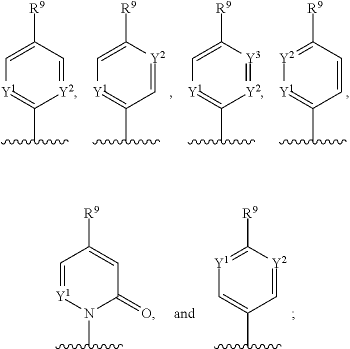

X¹ is selected from CR¹¹ and N; X² is selected from CR¹² and N; X³ is selected from CR¹³ and N, and X⁴′ is selected from CR¹⁴ and N, and wherein when X⁴′ is N the N may be an N-oxide, or X¹ is CR¹¹ and taken together with R forms a 5-membered heteroaryl ring which is optionally substituted with C(O)CF₃;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄ alkyl, halo-C₁₋₄alkyl, C₃₋₄cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅ alkyl may optionally be substituted one or more times with fluoro;
R³ is independently selected from hydrogen, C₁₋₄alkyl, and deuterated C₁₋₄alkyl;
each R⁴ and R⁶ are independently selected from hydrogen and C₁₋₄alkyl and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro; or
R⁵ and R¹⁰ together form a bond between the two carbons to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is —CONR¹⁵R¹⁶;
R¹¹, R¹², R¹³, and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3, and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —C(O)CF₃, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹⁵ and R¹⁶ are independently selected from hydrogen, C₁₋₄alkyl, and deuterated C₁₋₄alkyl; or a pharmaceutically acceptable salt or stereoisomer thereof.

In some embodiments are provided, compounds of formula (IA):

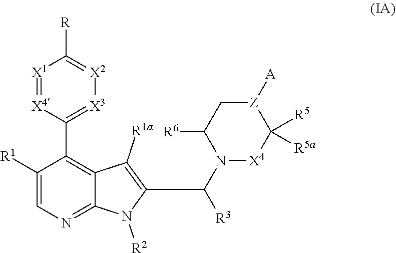

wherein A is selected from:
A is

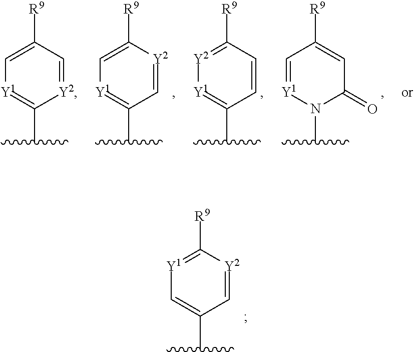

X¹ is selected from CR¹¹ and N; X² is selected from CR¹² and N; X³ is selected from CR¹³ and N, and X⁴ is selected from CR¹⁴ and N, and wherein when X⁴ is N the N may be an N-oxide, or X¹ is CR¹¹ and taken together with R forms a 5-membered heteroaryl ring which is optionally substituted with C(O)CF₃;
X⁴ is CO or CR⁴R⁴;
X⁶ is N or oxidized N;
Y¹ is selected from N and CR⁷;
Y² is selected from N and CR⁸;
Y³ is selected from NH and NHC⁸:
Z is selected from N and CR¹⁰;
R is selected from OH, CHF₂, NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)˜-O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄ alkyl, halo-C₁₋₄alkyl, C₃₋₄cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2 or R⁰ taken together with R¹¹ or R¹² forms a 5-6-membered heteroaryl or heterocyclic ring optionally substituted with COCF₃;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅ alkyl may optionally be substituted one or more times with fluoro;
R³ is independently selected from hydrogen and C₁₋₄alkyl;
each R⁴ and R⁶ are independently selected from hydrogen and C₁₋₄alkyl and C₁₋₄alkoxy,
wherein said C₁₋₄alkyl and C₁₋₄alkoxy may optionally be substituted with one or more halogens;
R⁵ is selected from hydrogen, fluoro and C₁₋₄alkyl; wherein said C₁₋₄alkyl may optionally be substituted with one or more halogens, and R^{5a} and R¹⁰ is hydrogen or fluoro; or
R⁵ and R^{5a} are both selected from fluoro or R⁵ and R^{5a} together form CO and R¹⁰ is hydrogen; or
R⁵ and R¹⁰ together form a bond, and R^{1a} is hydrogen;
R⁷ and R⁸ are independently selected from hydrogen, halogen, C₁₋₄alkyl and C₁₋₄alkoxy, —C₃-cycloalkyl, cyano wherein said C₁₋₄alkyl and C₁₋₄alkoxy may optionally be substituted with one or more halogen;
R⁹ is —CONR¹⁵R¹⁶;
R¹¹, R¹², R¹³, and R¹⁴ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3, and wherein R′ and R″ are each independently selected from hydrogen and C₁₋₄alkyl;
R¹⁵ and R¹⁶ are independently selected from hydrogen, methyl, and deuterated methyl;
or a pharmaceutically acceptable salt or stereoisomer thereof.

In another embodiment, the present disclosure provides a pharmaceutical composition comprising a compound of the present disclosure, and a pharmaceutically acceptable vehicle or excipient or pharmaceutically acceptable carrier(s).

In another embodiment, the present disclosure provides a method of treating an immune mediated disease or condition in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of modulating a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by a signal transducer and activator of transcription 6 (STAT6) protein activity in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In another embodiment, the present disclosure provides a method of treating a disease or condition mediated by interleukin 4 (IL-4) or interleukin 3 (IL-3) in a subject in need thereof, the method comprising administering to the subject a therapeutically effective amount of a compound of the present disclosure, or a pharmaceutically acceptable salt or stereoisomer thereof, or a pharmaceutical composition of the present disclosure.

In an embodiment the disclosure provides a method of preventing, treating or ameliorating a diseases characterized by Th2-mediated inflammation.

In another embodiment, the present disclosure provides a method for manufacturing a medicament for treating a disease or condition mediated by a signal transducer and activator of transcription 6 (STAT6) protein activity in a subject in need thereof, characterized in that a compound of the present disclosure, or a pharmaceutically acceptable salt or stereoisomer thereof, is used.

In another embodiment, the present disclosure provides a method for manufacturing a medicament for the treatment of a disease or condition mediated by a signal transducer and activator of transcription 6 (STAT6) protein in a subject.

In some embodiments, the present disclosure provides a compound of the present disclosure, or a pharmaceutically acceptable salt or stereoisomer thereof, for use in treating a disease or condition mediated by a signal transducer and activator of transcription 6 (STAT6) protein in a subject in need thereof.

In some embodiments, the present disclosure provides the compound of the present disclosure, or a pharmaceutically acceptable salt thereof, for use in therapy.

### BRIEF DESCRIPTION OF THE DRAWINGS

FIG. 1 presents the sensorgrams for Examples 1u2, 1x1, 1af26, 1y1, and 1af36 are shown below (where RU is plotted vs. time in seconds; dotted lines/points were excluded from the analysis due to effects from non-specific binding).

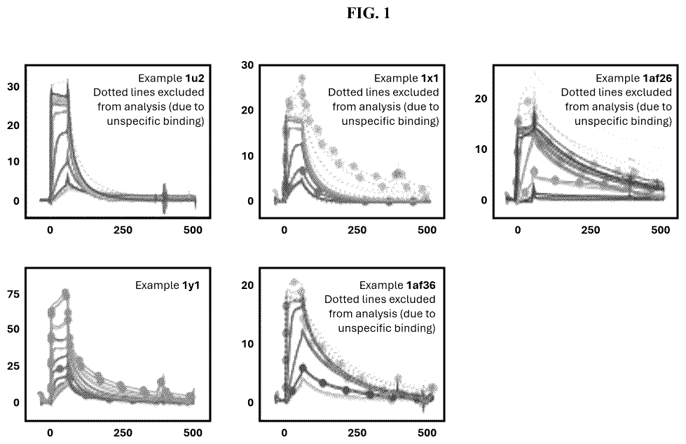

FIG. 2 presents the data from the human whole blood eotaxin-3 assay is shown below for Examples 1u2, 1x1, 1af26, and 1af36 (where the effect in % is plotted against the test concentration in M on a log-scale).

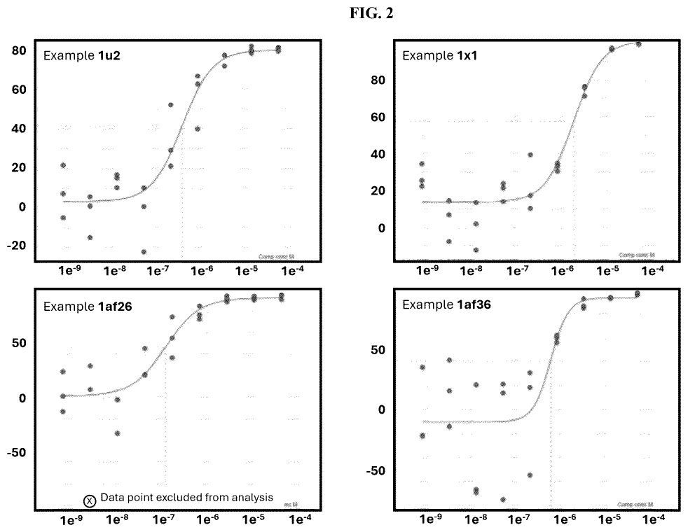

### DETAILED DESCRIPTION

### Definitions

Whenever the compound of formula (I) is mentioned herein it should be understood that the compound of formula (I), (II) and (III) are subgroups of the compound of formula (I) and that a statement related to the compound of formula (I) relates equally well to its subgroups.

The prefix “Cᵤ₋ᵥ” indicates that the following group has from u to v carbon atoms. For example, “C₁₋₄ alkyl” indicates that the alkyl group has from 1 to 4 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The term “C₁₋₄alkyl” is used herein to refer to hydrocarbon radical obtained when one hydrogen atom is removed from a branched or linear hydrocarbon. Said alkyl comprises (1-4) carbon atoms, 1-3 carbon atoms, 2-3 carbon atoms or 1-2 carbon atoms. The term includes the subclasses normal alkyl (n-alkyl), secondary and tertiary alkyl, such as methyl, ethyl, n-propyl, isopropyl, n-butyl, isobutyl, sec-butyl, and tert-butyl.

The term “(C₁-C₄)alkoxy” is used herein to refer to a radical of the formula —OR^{a}, wherein R^{a} is (C₁-C₄)alkyl as indicated herein, wherein the (C₁-C₄)alkyl group is appended to the parent molecular moiety through an oxygen atom, e.g. methoxy (—OCH₃), and ethoxy (—OCH₂CH₃).

The term “cyano” is used herein to refer to a —CN group attached to the parent molecular moiety through the carbon atom.

The term “(C₃-C₄)cycloalkyl is used herein to refer to a saturated (C₃-C₄)cycloalkane hydrocarbon radical, comprising 3-4 carbon atoms, e.g. cyclopropyl, or cyclobutyl.

The term “halogen” or “halo” is used herein to refer to chloro, bromo, fluoro, or iodo. In some embodiments, halogen is chloro, bromo, or fluoro. The term “halo-C₁₋₄alkyl” or “halo-C₁₋₄alkoxy” is used herein to refer to an “C₁₋₄alkyl” or “C₁₋₄alkoxy” group as defined above in which one or more hydrogen atoms have been replaced by halogen (e.g. fluorine). Examples of “halo-C₁₋₄alkyl” or “halo-C₁₋₄ alkoxy” include —CHF₂, —CF₃, —CH₂CF₃, —CF₂CF₃ or —OCF₃.

The term “deuterated C₁₋₄alkyl” is used herein to refer to an “C₁₋₄alkyl” group in which one or more hydrogen atoms have been replaced by deuterium. Examples of “deuterated C₁₋₄alkyl” include —CH₂D₂, —CHD₂ or -CD₃.

The term “C₁₋₄alkoxyl-C₁₋₄alkyl” is used herein the refer to “C₁₋₄alkyl” group in which one hydrogen atom have been replaced by (C₁-C₄)alkoxy. Examples of “C₁₋₄alkoxyl-C₁₋₄alkyl” “include methoxymethyl or methoxyethyl.

The term “(C₃-C₄)cycloalkyl-C₁₋₄alkyl” is used herein the refer to “C₁₋₄alkyl” group in which one hydrogen atom have been replaced by (C₃-C₄)cycloalkyl. Examples of “(C₃-C₄)cycloalkyl-C₁₋₄alkyl” include cyclopropylmethyl.

The term “phenyl-C₁₋₄alkyl” is used herein the refer to “C₁₋₄alkyl” group in which one hydrogen atom have been replaced by phenyl. Examples of “phenyl-C₁₋₄alkyl” include benzyl.

“Aromatic ring” or “aryl” refers to an aromatic carbocyclic group having a single ring (e.g., monocyclic) or multiple rings (e.g., bicyclic or tricyclic) including fused systems. An aryl may have 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Heteroaromatic ring” or “heteroaryl” refers to an aromatic group having a single ring, multiple rings or multiple fused rings, with one or more ring heteroatoms independently selected from nitrogen, oxygen, and sulfur unless specified otherwise. A heteroaryl may include 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thiophenyl (i.e., thienyl), triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclic ring” or “heterocyclyl” refers to a saturated or partially unsaturated cyclic alkyl group, with one or more ring heteroatoms independently selected from nitrogen, oxygen, and sulfur, unless specified otherwise. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. A heterocyclyl may have 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl.

If substituents are described as being independently selected from a group, each substituent is selected independent of the other. Each substituent may therefore be identical or different from the other substituent(s).

The term “optionally substituted” means “unsubstituted or substituted.”, In some embodiments, formulas described herein encompasses compounds containing the specified optional substituent(s) as well as compounds that do not contain the optional substituent(s).

In certain embodiments, as used herein, the phrase “one or more” refers to one to five. In certain embodiments, as used herein, the phrase “one or more” refers to one to three.

As used herein whenever a molecular drawing of a substituent contains an arrow—the arrow indicates the bond attaching the substituent to the rest of the molecule.

The term “pharmaceutically acceptable salt” is intended to indicate non-toxic salts including a free base form of a compound that possesses the desired pharmacological activity of the free base. These salts may be derived from an appropriate basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid.

Pharmaceutically acceptable salts of compounds of formula (I) comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which are incorporated herein by reference.

Compounds of the disclosure containing an amine function may also form N-oxides. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The term “solvate” is intended to indicate a species formed by interaction between a compound, e.g. a compound of formula (I) or a pharmaceutically acceptable salt or stereoisomer thereof, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

“Administering” refers to oral administration, administration as a suppository, topical contact (e.g., transdermal), parenteral, intravenous, intraperitoneal, intramuscular, intralesional, intranasal, inhaled, intradermal, and/or subcutaneous administration, intrathecal administration, and/or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

### Compounds

Provided herein are compound that modulate the activity of STAT6.

In certain embodiments, provided is a compound of formula (I):

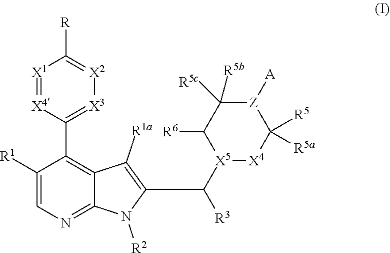

A is selected from

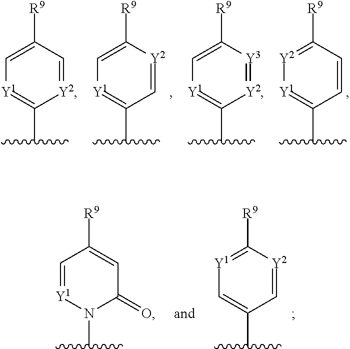

X¹ is selected from CR¹¹ and N; X² is selected from CR¹² and N; X³ is selected from CR¹³ and N, and X⁴′ is selected from CR¹⁴ and N, and wherein when X⁴′ is N the N may be an N-oxide, or X¹ is CR¹¹ and taken together with R forms a 5-membered heteroaryl ring which is optionally substituted with C(O)CF₃;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted one or more times with substituents independently selected from halogen, C₁₋₄ alkyl, halo-C₁₋₄alkyl, C₃₋₄cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅ alkyl may optionally be substituted one or more times with fluoro;
R³ is independently selected from hydrogen and C₁₋₄alkyl;
each R⁴ and R⁶ are independently selected from hydrogen and C₁₋₄alkyl and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄ alkoxy may optionally be substituted with one or more halogen;
R¹⁰ is selected from hydrogen and fluoro; or
R⁵ and R¹⁰ together form a bond between the two carbons to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is —CONR¹⁵R¹⁶;
R¹¹, R¹², R¹³ and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3, and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —C(O)CF₃, —NR′R″, —CONR′R″ and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl;
R¹⁵ and R¹⁶ are independently selected from hydrogen, C₁₋₄alkyl, and deuterated C₁₋₄alkyl; or a pharmaceutically acceptable salt or stereoisomer thereof.

In certain embodiments, R³ is methyl.

In one embodiment the disclosure relates to a compound of formula (I′)

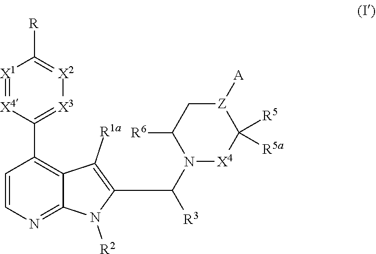

wherein A is selected from

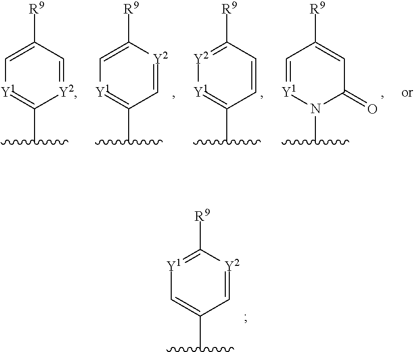

wherein X¹, X², X³, X^{4′}, X⁵, Y¹, Y², Z, R, R¹, R² R⁵, R^{5a}, R⁶ and R⁹ are as defined herein and R³ is C₁₋₄alkyl, or pharmaceutically acceptable salts thereof.

In a further embodiment, provided is a compound of formula (IIA), (IIB), (IIC), (IID) or (IIE):

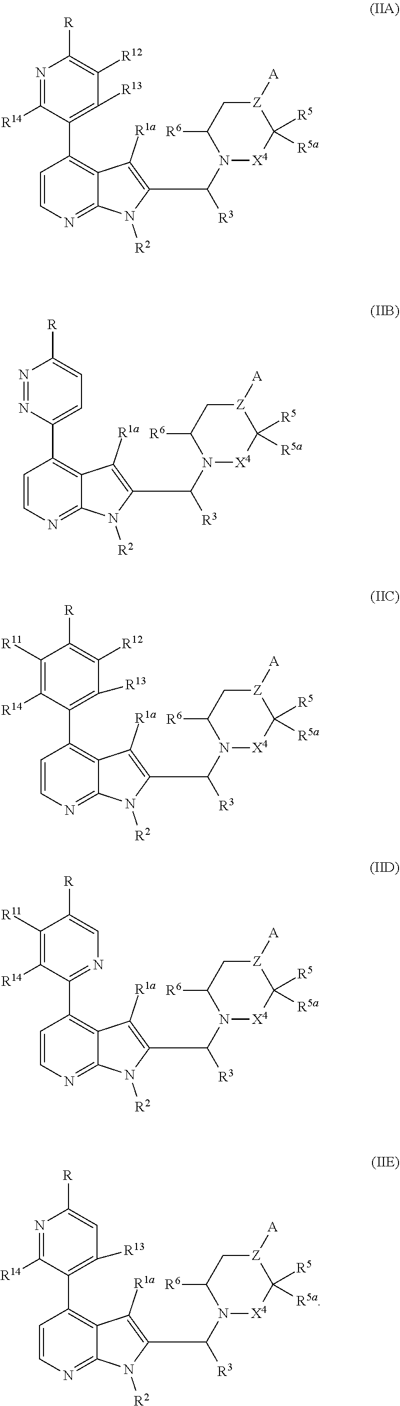

In a further embodiment, X¹ is selected from CR¹¹ and N; X² is selected from CR¹² and N; X³ is selected from CR¹³ and N and X⁴ is selected from CR⁴ and N.

In a further embodiment, X¹ is N, X² is CR¹², X³ is CR¹³ and X⁴ is CR¹⁴.

In a further embodiment, X¹ is CR¹¹, X² is CR¹², X⁴ is CR¹⁴ and X³ is N.

In a further embodiment, X¹ is CR¹¹, X² is CR¹², X³ is CR¹³ and X⁴ is CR¹⁴.

In a further embodiment, X¹ is N; X² is selected from CR¹², X³ is N and X⁴ is CR¹⁴.

In a further embodiment, X¹ is N, X² is N, X³ is CR¹³ and X⁴ is CR¹⁴.

In a further embodiment, X¹, X², X³ is N and X⁴ is CR¹⁴.

In a further embodiment, X¹ is CR¹¹, X² is CR¹², and X³ and X⁴ is N.

In a further embodiment, X¹ is N, X² is CR¹², and X³ and X⁴ is N.

In a further embodiment, X¹ is CR¹¹, X² is N, X³ is N and X⁴ is N.

In a further embodiment, each of R¹¹, R¹², R¹³, R¹⁴, if present, are all hydrogen. In further embodiment, each of R¹¹, R¹², R¹³, R¹⁴, if present, are independently selected from hydrogen, hydroxy, C₁₋₄alkyl optionally substituted with 1-3 halo or hydroxy, C₁₋₄alkoxy optionally substituted with halo, cyano, halo, —CO—CO₂R′, —CO₂R′, —SO₂—C₁₋₄alkyl, —NR′R″, and tetrazolyl.

In a further embodiment, only one of R¹¹, R¹², R¹³, R¹⁴ are selected from hydroxy, C₁₋₄alkyl optionally substituted with 1-3 halo or hydroxy, C₁₋₄alkoxy optionally substituted with halo, cyano, halo, —CO—CO₂R′, —CO₂R′, —SO₂—C₁₋₄alkyl, —NR′R″, and tetrazolyl and the remaining of R¹¹, R¹², R¹³, R¹⁴ are each hydrogen.

In a further embodiment, each of R¹¹, R¹², R¹³, R¹⁴ are each independently selected from hydrogen methyl, methoxy, fluoro, CF₃, CHF₂, —OCF₃, —C(O)C(O)OH, —COOH, —COOMe, —COOEt, —SO₂Me, N(CH₃)₂, tetrazolyl, and —C(O)CF₃.

In a further embodiment, Z is N.

In a further embodiment, Z is CR¹⁰ and R⁵ and R¹⁰ together form a bond.

In a further embodiment, Z is CR¹⁰ and R¹⁰ is hydrogen.

In some embodiments, R³ is selected from hydrogen and C₁₋₄alkyl.

In some embodiments, R³ is hydrogen.

In some embodiments, R³ is C₁₋₄alkyl. In some embodiments, R³ is methyl.

In some embodiments, A is selected from

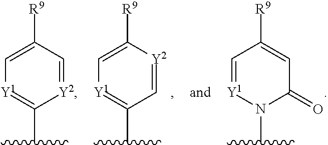

In some embodiments, A is

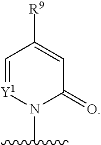

In some embodiments, A is selected from

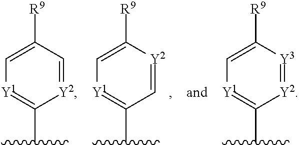

In some embodiments, A is selected from

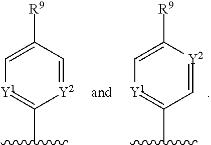

In some embodiments, Y¹ is N and Y² is CR⁸.

In some embodiments, Y¹ is N and Y² is N.

In some embodiments, Y¹ is CR⁷ and Y² is CR⁸.

In some embodiments, Y¹ is CR⁷ and Y² is N.

In some embodiments, A is selected from

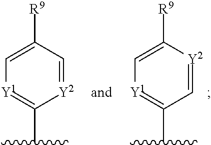

Y¹ is CR⁷; and Y² is N.

In some embodiments, A is selected from

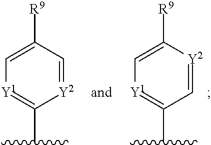

Y¹ is CR⁷; and Y² is CR⁸.

In some embodiments, A is selected from

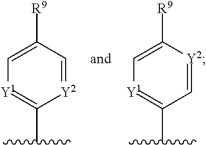

Y¹ is CR⁷; and Y² is CR⁸.

In some embodiments, A is

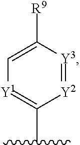

Y¹ is CR⁷, Y² is N or CR⁸, and Y³ is N.

In a further embodiment, Y¹ is CR⁷ and Y² is CR⁸.

In a further embodiment, Y¹ is CR⁷ and Y² is N.

In a further embodiment, Y¹ and Y² is N.

In a further embodiment,
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen;
(e) one of R⁷ and R⁸ is halogen and the other is hydrogen;
(f) one of R⁷ and R⁸ is cyclopropyl; or
(g) one R⁷ and R⁸ is cyano.

In a further embodiment, one or both of R⁷ and R⁸ is hydrogen.

In a further embodiment, one of R⁷ and R⁸ is hydrogen and the other is fluoro, cyano, or cyclopropyl.

In a further embodiment, R is NHR⁰ and R⁰ is selected from C₁₋₄alkyl, —(CH₂)ₙ—O—C₁₋₄alkyl, —(CH₂)ₙ—CO—C₁₋₄alkyl and —(CH₂)ₙ—CO₂R′ where n is 0, 1 or 2. In a further embodiment, the disclosure relates to a compound as above wherein R⁹ is selected from CON(CH₃)₂ and —CON(CD₃)₂.

In a further embodiment, R⁰ is selected from hydrogen, C₁₋₄alkyl, —(CH₂)ₙ—O—R′, —(CH₂)ₙ—CO—C₁₋₄alkyl and —(CH₂)ₙ—CO₂R′.

In some embodiments, the group NHR⁰ is selected from NH₂, NHCH₃, NHC(O)CH₃, NH(CH₃)₂OCH₃, and NHCH₃CO₂H.

In some embodiments, A is phenyl, pyridyl, or pyridazinyl each of which is optionally substituted with one or two of substituents independently selected from halo, C₁₋₄alkyl optionally substituted with one to 3 halo, C₁₋₄alkoxy, C₃₋₄cycloalkyl, or cyano.

In some embodiments, R″, when present, is selected from hydrogen, halogen, C₁₋₄alkyl, C₃₋₄ cycloalkyl, C₁₋₄alkoxy, hydroxy, cyano, —NR′R″, —CONR′R″, —CO₂R′, and —CO—(CH₂)ₘOH, wherein said C₁₋₄alkyl is optionally substituted one or more times with a substituent independently selected from halogen, hydroxy, and C₁₋₄alkoxy, and wherein m is 1, 2 or 3, and wherein R′ and R″ are each independently selected from hydrogen and C₁₋₄alkyl.

In some embodiments, R¹¹, when present, is selected from hydrogen, halogen, C₁₋₄alkyl, C₃₋₄ cycloalkyl, C₁₋₄alkoxy, cyano, and —CO—(CH₂)ₘOH, wherein said C₁₋₄alkyl is optionally substituted one or more times with a substituent independently selected from halogen and C₁₋₄alkoxy, and wherein m is 1, 2 or 3.

In some embodiments, R¹¹, when present, is selected from hydrogen, halogen, —CH₂—OMe, cyclopropyl, methyl, —CF₃, —CO—(CH₂)₃—OH, —CH₂—O-tBu, and methoxy.

In some embodiments, R is NHR⁰; and R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted one or more times with a substituent independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, wherein said C₁₋₄alkyl may optionally be substituted one or more times with a substituent independently selected from halogen and C₁₋₄alkoxy, and wherein R′ and R″ are each independently selected from hydrogen and C₁₋₄alkyl.

In some embodiments, R is NHR⁰; and R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted with a halo-C₁₋₄alkyl.

In some embodiments, R is NHR⁰; and R¹¹ and R⁰ together with the atoms attached thereto form a 5-6 membered heteroaromatic ring containing one or two N atoms, wherein said 5-6 membered heteroaromatic ring containing one or two N atoms is optionally substituted with a C₁₋₄haloalkyl.

In some embodiments, R is NHR⁰; and R¹¹ and R⁰ together with the atoms attached thereto form a pyrazole, wherein said pyrazole is optionally substituted with a C₁₋₄haloalkyl.

In some embodiments, R is NHR⁰; and R¹¹ and R⁰ together with the atoms attached thereto form a 5-6 membered heterocyclic ring containing one or two N atoms.

In some embodiments, R is NHR⁰; and R¹¹ and R⁰ together with the atoms attached thereto form a pyrrolidine or piperidine.

In some embodiments, each of R¹² and R¹³, when present, is independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, and —CO—(CH₂)ₘOH, wherein said C₁₋₄alkyl is optionally substituted one or more times with a substituent independently selected from halogen, hydroxy, and C₁₋₄alkoxy, and wherein m is 1, 2 or 3, and wherein R′ and R″ are each independently selected from hydrogen and C₁₋₄alkyl.

In some embodiments, each of R¹² and R¹³, when present, is independently selected from hydrogen, halogen, C₁₋₄alkyl, C₃₋₄cycloalkyl, C₁₋₄alkoxy, cyano, and —CO—(CH₂)ₘOH, wherein said C₁₋₄alkyl is optionally substituted one or more times with a substituent independently selected from halogen and C₁₋₄alkoxy, and wherein m is 1, 2, or 3.

In some embodiments, each of R¹² and R¹³, when present, is independently selected from hydrogen, halogen, C₁₋₄alkyl, C₁₋₄alkoxy, and cyano.

In some embodiments, each of R¹² and R¹³, when present, is independently selected from hydrogen, halogen, methyl, methoxy, and cyano.

In some embodiments, R¹² and R¹³ are both hydrogen.

In some embodiments, R¹¹, R¹² and R¹³ are all hydrogen.

In some embodiments, R is NHR⁰; and R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R, wherein said phenyl is optionally substituted one or more times with a substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1, or 2.

In some embodiments, R is NHR⁰; and R⁰ is selected from C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—C₁₋₄alkyl, —(CH₂)ₙ—CO—R′, and —(CH₂)ₙ—CO₂R, wherein said phenyl is optionally substituted one or more times with a substituents independently selected from halogen, C₁₋₄alkyl, halo-C₁₋₄alkyl, C₃₋₄cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl, and n is 0, 1 or 2.

In some embodiments, R is NHR⁰; and R⁰ is selected from hydrogen, C₁₋₄alkyl, C₃₋₄ cycloalkyl, C₃₋₄ cycloalkyl-C₁₋₄alkyl, phenyl-C₁₋₄alkyl, and —CO₂R′, wherein said phenyl is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl.

In some embodiments, R is NHR⁰; and R⁰ is selected from C₁₋₄alkyl, C₃₋₄ cycloalkyl, C₃₋₄ cycloalkyl-C₁₋₄alkyl, phenyl-C₁₋₄alkyl, and —CO₂R′, wherein said phenyl is optionally substituted one or more times with a substituent independently selected from halogen, C₁₋₄alkyl, halo-C₁₋₄alkyl, C₃₋₄ cycloalkyl, hydroxy, C₁₋₄alkoxy, cyano, —NR′R″, —CONR′R″, and —CO₂R′, and wherein R′ and R″ are each independently selected from hydrogen and C₁₋₄alkyl.

In some embodiments, R is NHR⁰; and R⁰ is selected from hydrogen, C₁₋₄alkyl, C₃₋₄ cycloalkyl, C₃₋₄ cycloalkyl-C₁₋₄alkyl, phenyl-C₁₋₄alkyl, and —CO₂R′, wherein R′ is C₁₋₄alkyl, and wherein said phenyl is optionally substituted one or more times with a substituent independently selected from halogen.

In some embodiments, R is NHR⁰; and R⁰ is selected from C₁₋₄alkyl, C₃₋₄ cycloalkyl, C₃₋₄ cycloalkyl-C₁₋₄alkyl, phenyl-C₁₋₄alkyl, and —CO₂R′, wherein R′ is C₁₋₄alkyl, and wherein said phenyl is optionally substituted one or more times with a substituent independently selected from halogen. In some embodiments, R is NHR⁰; and R⁰ is selected from hydrogen and C₁₋₄alkyl.

In some embodiments, R is NHR⁰; and R⁰ is C₁₋₄alkyl.

In some embodiments, R is NHR⁰; and R⁰ is hydrogen.

In some embodiments, R is NHR⁰; and R⁰ is selected from hydrogen, methyl, ‘butyl, cyclopropyl, —CO₂Me, —CO₂Et, —CO₂tBu, —CH₂-phenyl, and —CH₂-cyclopropyl, wherein said phenyl is optionally substituted one or more times with a substituent independently selected from halogen

In a further embodiment, R¹ is hydrogen, R² is hydrogen and R³ is selected from hydrogen and methyl.

In some embodiments, the halogen in b) c) f) and h) is fluoro.

In some embodiments, Z is N.

In some embodiments, Z is CR¹⁰.

In some embodiments, Z is CR¹⁰ and R¹⁰ is selected from hydrogen and fluoro.

In some embodiments, Z is CR¹⁰ and R¹⁰ is hydrogen.

In some embodiments, Z is CR¹⁰ and R⁵ and R¹⁰ together form a bond between the two carbons to which the are attached.

In some embodiments, R⁵ is selected from hydrogen, halogen, C₁₋₄alkyl, and C₁₋₄alkoxy, wherein said C₁₋₄alkyl and C₁₋₄alkoxy may optionally be substituted with one or more halogen.

In some embodiments, R⁵ is selected from hydrogen and halogen.

In some embodiments, R⁵ is selected from hydrogen and fluoro.

In some embodiments, R^{5a} is hydrogen.

In some embodiments, one of R⁵ and R^{5a} is hydrogen and the other one is fluoro.

In some embodiments, R⁵ and R^{5a} are both fluoro.

In some embodiments, R⁵ and R^{5a} together with the atom attached thereto form a CO group.

In some embodiments, R^{5b} and R^{5c} are independently selected from hydrogen and fluoro.

In some embodiments, R^{5b} and R^{5c} are both hydrogen.

In some embodiments, R^{5b} and R^{5c} are both hydrogen; R⁵ is selected from hydrogen, halogen, C₁₋₄alkyl, and C₁₋₄alkoxy, wherein said C₁₋₄alkyl and C₁₋₄alkoxy may optionally be substituted with one or more halogen; and R^{5a} is hydrogen.

In some embodiments, R^{5b} and R^{5c} are both hydrogen, and R⁵ and R^{5a} are both fluoro.

In some embodiments, R⁵, R^{5a}, R^{5b}, and R^{5c} are not all hydrogen.

In some embodiments, X⁴ is CO.

In some embodiments, X⁴ is CR⁴R⁴ and each R⁴ is selected from hydrogen and C₁₋₄alkyl, wherein said C₁₋₄alkyl is optionally substituted with one or more halogen.

In some embodiments, X⁴ is CR⁴R⁴ and each R⁴ is selected from hydrogen and C₁₋₄alkyl.

In some embodiments, X⁴ is CR⁴R⁴ and each R⁴ is selected from hydrogen and methyl.

In some embodiments, X⁵ is selected from N and oxidized N. In some embodiments, X⁵ is N. In some embodiments, X⁵ is oxidized N.

In some embodiments, R⁶ is selected from hydrogen and C₁₋₄alkyl, wherein said C₁₋₄alkyl is optionally substituted with one or more halogen.

In some embodiments, R⁶ is selected from hydrogen and C₁₋₄alkyl.

In some embodiments, R⁶ is selected from hydrogen and methyl.

In some embodiments, at least one of R¹⁰, R⁵, R^{5a}, R^{5b}, R^{5c}, R⁴, and R⁶ is not hydrogen. In some embodiments, at least one of R⁵, R^{5a}, R^{5b}, R^{5c}, R⁴, and R⁶ is not hydrogen. In some embodiments, at least one of R⁵, R^{5a}, R^{5b}, R^{5c}, and R⁶ is not hydrogen.

In some embodiments, the

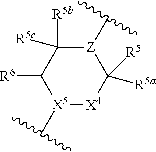

moiety is

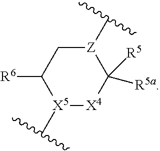

In some embodiments, the

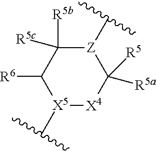

moiety is selected from:

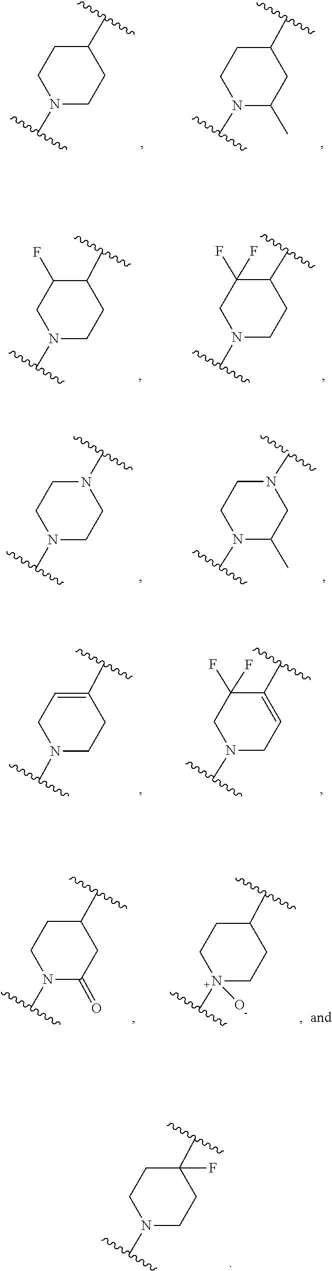

In some embodiments, the

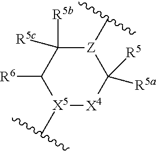

moiety is selected from:

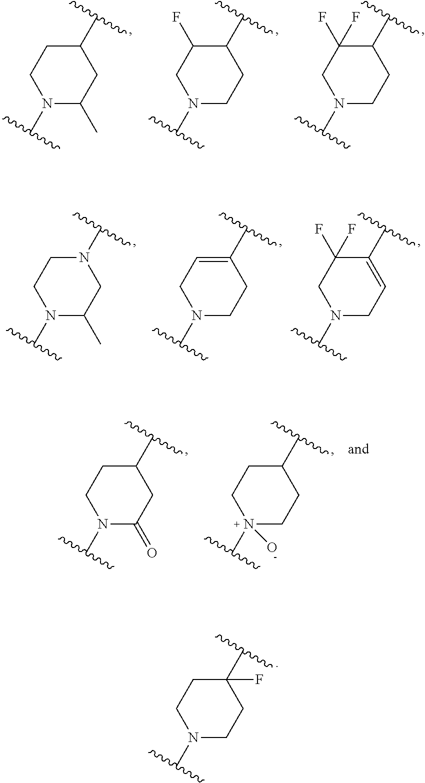

In some embodiments, the

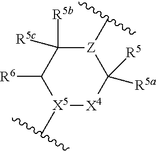

moiety is

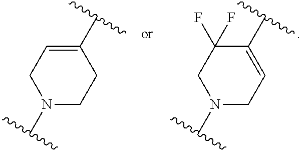 [SMILES: *C1=CCN(*)CC1 ; *C1=CCN(*)CC1(F)F]

In some embodiments, the

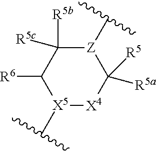

moiety is selected from:

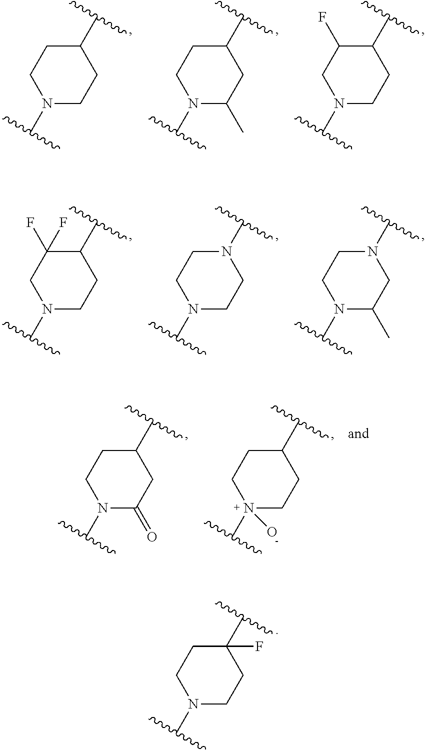

In some embodiments, the

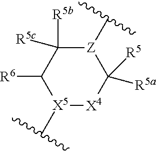

moiety is selected from:

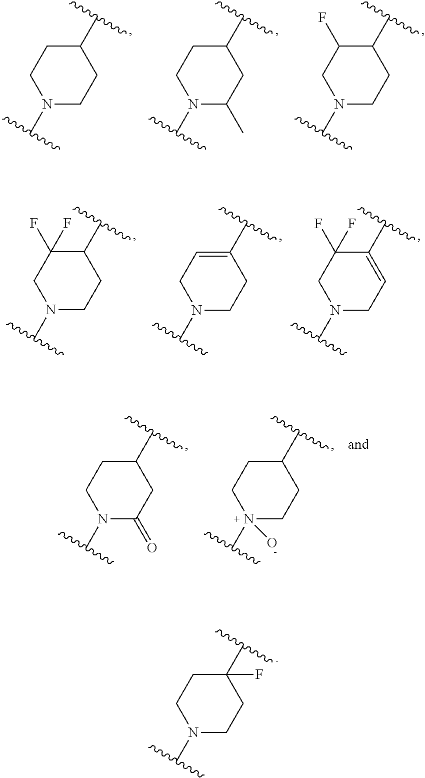

In some embodiments, the

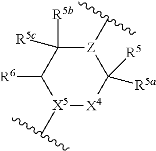

moiety is

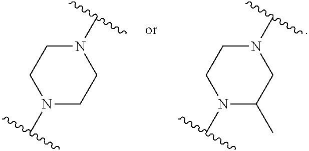

In some embodiments, R⁹ is —CONR¹⁴R¹⁵, wherein R¹⁴ and R¹⁵ are each independently selected from hydrogen, C₁₋₄alkyl, and deuterated C₁₋₄alkyl.

In some embodiments, R⁹ is —CONR¹⁴R¹⁵, wherein R¹⁴ and R¹⁵ are each independently selected from C₁₋₄alkyl and deuterated C₁₋₄alkyl.

In some embodiments, R⁹ is —NR¹⁴CO₂R¹⁵, wherein R¹⁴ and R¹⁵ are each independently selected from C₁₋₄alkyl and deuterated C₁₋₄alkyl.

In some embodiments, R⁹ is —CONR¹⁴R¹⁵; and R¹⁴ and R¹⁵ are each independently selected from methyl and deuterated methyl.

In some embodiments, R⁹ is selected from CON(CH₃)₂, CON(CH₂CH₃)₂, CON(CH₂CH₂CH₃)₂, CON(CD₃)₂, and CONH(CH₃).

In some embodiments, R⁹ is selected from CON(CH₃)₂ or CON(CD₃)₂.

In some embodiments, R¹ and R^{1a} are each independently selected from hydrogen, fluoro, and C₁₋₄alkyl.

In some embodiments, R¹ and R^{1a} are both hydrogen.

In some embodiments, R² is selected from hydrogen, C₁₋₄alkyl, and deuterated C₁₋₄alkyl; wherein said C₁₋₄alkyl is optionally substituted with one or more halogen.

In some embodiments, R² is selected from C₁₋₄alkyl and deuterated C₁₋₄alkyl; wherein said C₁₋₄alkyl is optionally substituted with one or more halogen.

In some embodiments, R² is methyl. In some embodiments, R² is hydrogen.

In certain embodiments, provided is a compound selected from Table 1, or a pharmaceutically acceptable salt or stereoisomer thereof:

| Example | Structure | Name |
|---|---|---|
| 1aa2 1aa2′ | 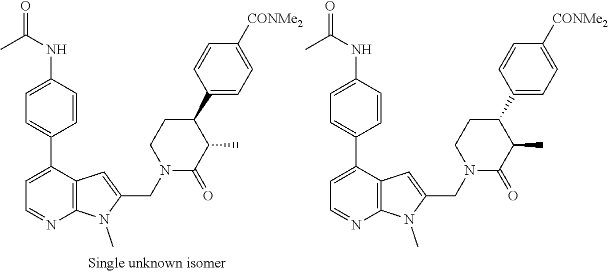 | N,N-Dimethyl-4-[(3S,4R)-1-[[4-(4- acetamidophenyl)-1-methyl- pyrrolo[2,3-b]pyridin-2-yl]methyl]-3- methyl-2-oxo-4-piperidyl]benzamide and N,N-dimethyl-4-[(3R,4S)-1-[[4-(4- acetamidophenyl)-1-methyl- pyrrolo[2,3-b]pyridin-2-yl]methyl]-3- methyl-2-oxo-4-piperidyl]benzamide |
| 1ad1 | 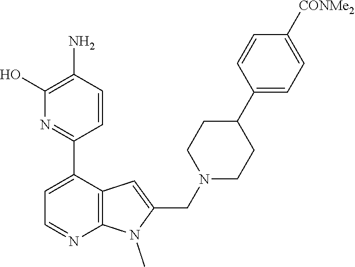 | 4-(1-((4-(5-Amino-6-hydroxypyridin- 2-yl)-1-methyl-1H-pyrrolo[2,3- b]pyridin-2-yl)methyl)piperidin-4-yl)- N,N-dimethylbenzamide |
| 1ad2 | 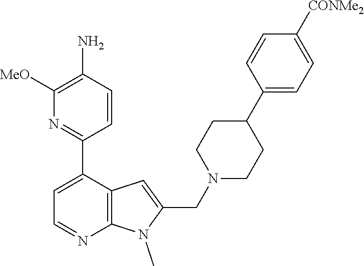 | 4-(1-((4-(5-Amino-6-methoxypyridin- 2-yl)-1-methyl-1H-pyrrolo[2,3- b]pyridin-2-yl)methyl)piperidin-4-yl)- N,N-dimethylbenzamide |
| 1ad3 | 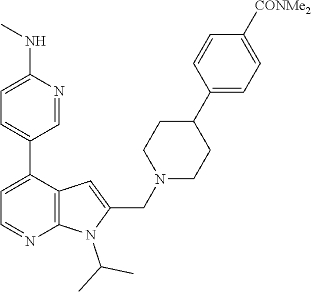 | 4-[1-[[1-iso-Propyl-4-[6- (methylamino)-3-pyridyl]pyrrolo[2,3- b]pyridin-2-yl]methyl]-4-piperidyl]- N,N-dimethyl-benzamide |
| 1ae4 1ae5 | 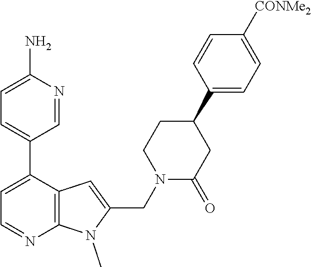 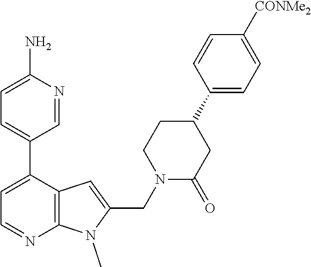 | (R)-N,N-Dimethyl-4-[1-[[4-(6-amino- 3-pyridyl)-1-methyl-pyrrolo[2,3- b]pyridin-2-yl]methyl]-2-oxo-4- piperidyl]benzamide and (S)-N,N- dimethyl-4-[1-[[4-(6-amino-3-pyridyl)- 1-methyl-pyrrolo[2,3-b]pyridin-2- yl]methyl]-2-oxo-4- piperidyl]benzamide |
| 1ae7 | 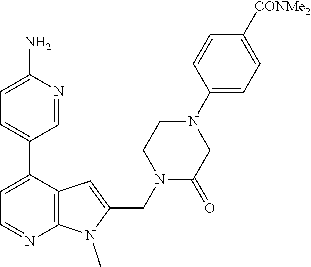 | 4-[4-[[4-(6-Amino-3-pyridyl)-1- methyl-pyrrolo[2,3-b]pyridin-2- yl]methyl]-3-oxo-piperazin-1-yl]-N,N- dimethyl-benzamide |
| 1ae10 | 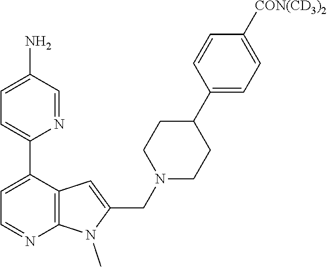 | 4-[1-[[4-(5-Amino-2-pyridyl)-1- methyl-pyrrolo[2,3-b]pyridin-2- yl]methyl]-4-piperidyl]-N,N- bis(trideuteriomethyl)benzamide |
| 1ae14 | 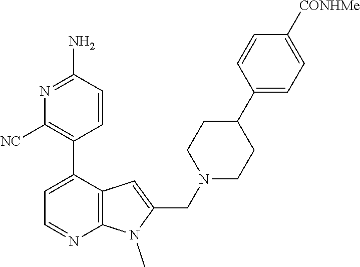 | 4-[1-[[4-(6-Amino-2-cyano-3-pyridyl)- 1-methyl-pyrrolo[2,3-b]pyridin-2- yl]methyl]-4-piperidyl]-N-methyl- benzamide |
| 1ae15 | 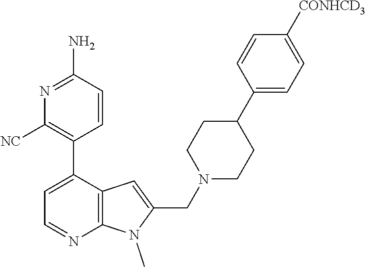 | 4-[1-[[4-(6-Amino-2-cyano-3-pyridyl)- 1-methyl-pyrrolo[2,3-b]pyridin-2- yl]methyl]-4-piperidyl]-N- (trideuteriomethyl)benzamide |
| 1ae19 | 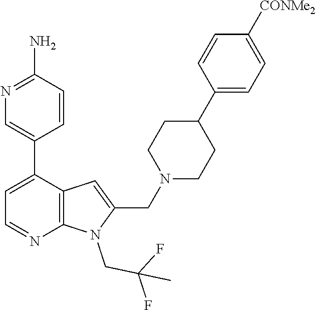 | 4-[1-[[1-(2,2-Difluoropropyl)-4-[6- (methylamino)-3-pyridyl]pyrrolo[2,3- b]pyridin-2-yl]methyl]-4-piperidyl]- N,N-dimethyl-benzamide |
| 1ae22 | 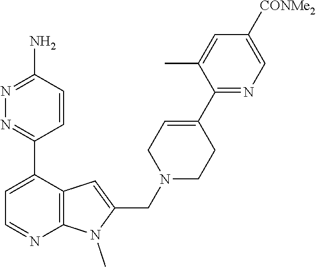 | 1′-((4-(6-Aminopyridazin-3-yl)-1- methyl-1H-pyrrolo[2,3-b]pyridin-2- yl)methyl)-N,N,3-trimethyl-1′,2′,3′,6′- tetrahydro-[2,4′-bipyridine]-5- carboxamide |
| 1af7 1af8 | 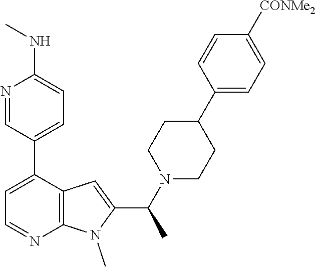 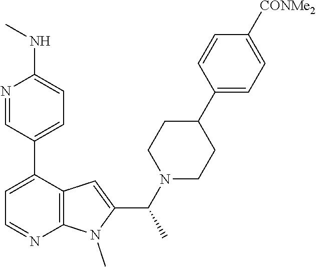 | (S)-N,N-Dimethyl-4-(1-(1-(1-methyl- 4-(6-(methylamino)pyridin-3-yl)-1H- pyrrolo[2,3-b]pyridin-2- yl)ethyl)piperidin-4-yl)benzamide and (R)-N,N-Dimethyl-4-(1-(1-(1-methyl- 4-(6-(methylamino)pyridin-3-yl)-1H- pyrrolo[2,3-b]pyridin-2- yl)ethyl)piperidin-4-yl)benzamide |
| 1af11 1af12 | 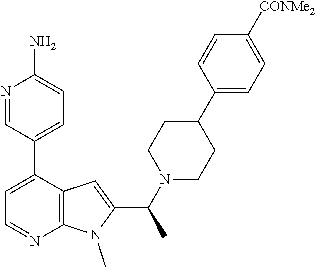 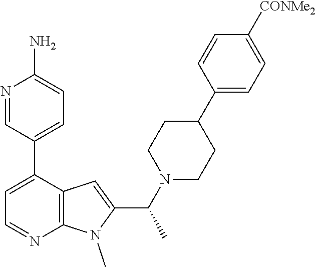 | (S)-4-(1-(1-(4-(6-amino-pyridin-3-yl)- 1-methyl-1H-pyrrolo[2,3-b]pyridin-2- yl)ethyl)-piperidin-4-yl)-N,N- dimethyl-benzamide and (R)-4-(1-(1- (4-(6-aminopyridin-3-yl)-1-methyl- 1H-pyrrolo[2,3-b]pyridin-2- yl)ethyl)piperidin-4-yl)-N,N- dimethylbenzamide |
| 1af13 1af14 | 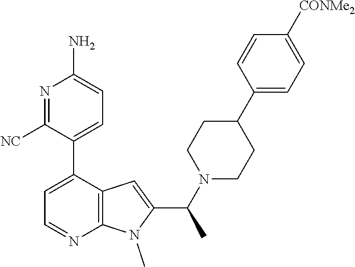 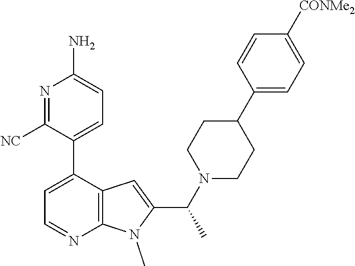 | (S)-4-(1-(1-(4-(6-amino-2- cyanopyridin-3-yl)-1-methyl-1H- pyrrolo[2,3-b]pyridin-2- yl)ethyl)piperidin-4-yl)-N,N- dimethylbenzamide and (R)-4-(1-(1- (4-(6-amino-2-cyanopyridin-3-yl)-1- methyl-1H-pyrrolo[2,3-b]pyridin-2- yl)ethyl)piperidin-4-yl)-N,N- dimethylbenzamide |
| 1af15 1af16 | 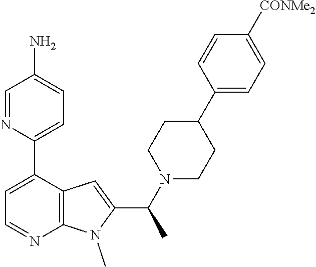 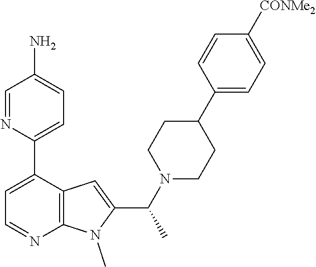 | (S)-4-(1-(1-(4-(5-amino-pyridin-2-yl)- 1-methyl-1H-pyrrolo[2,3-b]pyridin-2- yl)-ethyl)piperidin-4-yl)-N,N- dimethylbenzamide and (R)-4-(1-(1- (4-(5-aminopyridin-2-yl)-1-methyl- 1H-pyrrolo[2,3-b]pyridin-2- yl)ethyl)piperidin-4-yl)-N,N- dimethylbenzamide |
| 1af17 1af18 | 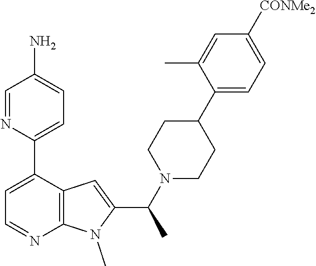 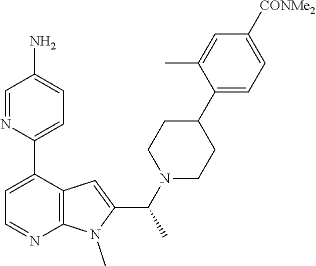 | (S)-4-(1-(1-(4-(5-aminopyridin-2-yl)- 1-methyl-1H-pyrrolo[2,3-b]pyridin-2- yl)ethyl)piperidin-4-yl)-N,N,3- trimethylbenzamide and (R)-4-(1-(1- (4-(5-aminopyridin-2-yl)-1-methyl- 1H-pyrrolo[2,3-b]pyridin-2- yl)ethyl)piperidin-4-yl)-N,N,3- trimethylbenzamide |
| 1af19 1af20 | 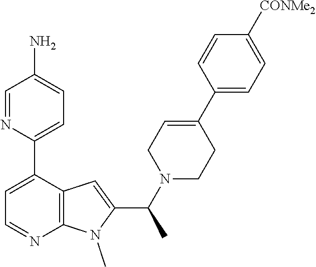 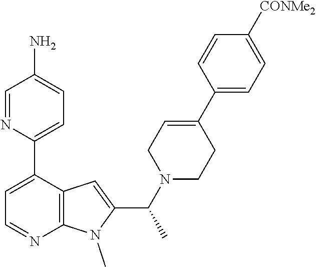 | (S)-4-(1-(1-(4-(5-Aminopyridin-2-yl)- 1-methyl-1H-pyrrolo[2,3-b]pyridin-2- yl)ethyl)-1,2,3,6-tetrahydropyridin-4- yl)-N,N-dimethylbenzamide and (R)-4- (1-(1-(4-(5-aminopyridin-2-yl)-1- methyl-1H-pyrrolo[2,3-b]pyridin-2- yl)ethyl)-1,2,3,6-tetrahydropyridin-4- yl)-N,N-dimethylbenzamide |
| 1af21 1af23 | 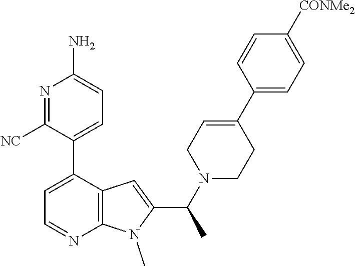 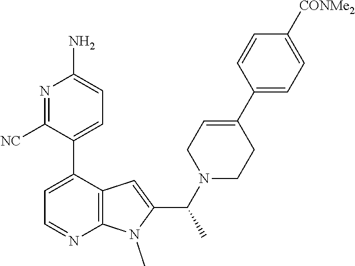 | S)-4-(1-(1-(4-(6-amino-2- cyanopyridin-3-yl)-1-methyl-1H- pyrrolo[2,3-b]pyridin-2-yl)ethyl)- 1,2,3,6-tetrahydropyridin-4-yl)-N,N- dimethylbenzamide and (R)-4-(1-(1- (4-(6-amino-2-cyanopyridin-3-yl)-1- methyl-1H-pyrrolo[2,3-b]pyridin-2- yl)ethyl)-1,2,3,6-tetrahydropyridin-4- yl)-N,N-dimethylbenzamide |
| 1af22 1af24 | 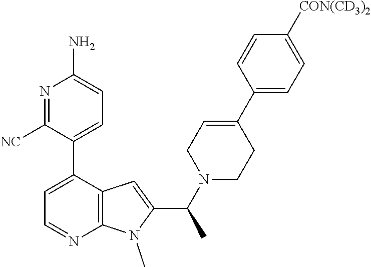 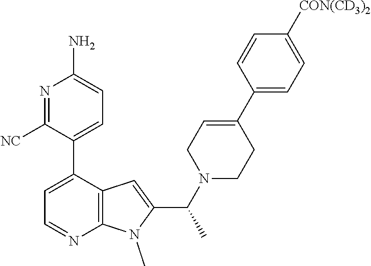 | (R)-4-(1-(1-(4-(6-Amino-2- cyanopyridin-3-yl)-1-methyl-1H- pyrrolo[2,3-b]pyridin-2-yl)ethyl)- 1,2,3,6-tetrahydropyridin-4-yl)-N,N- bis(methyl-d₃)benzamide and (S)-4-(1- (1-(4-(6-amino-2-cyanopyridin-3-yl)- 1-methyl-1H-pyrrolo[2,3-b]pyridin-2- yl)ethyl)-1,2,3,6-tetrahydropyridin-4- yl)-N,N-bis(methyl-d₃)benzamide |
| 1af25 1af26 | 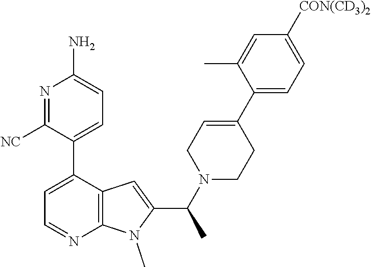 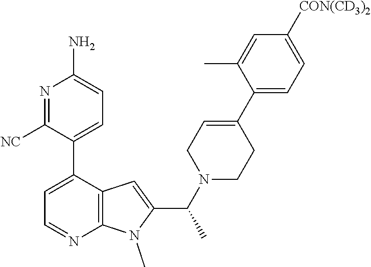 | (S)-4-(1-(1-(4-(6-amino-2- cyanopyridin-3-yl)-1-methyl-1H- pyrrolo[2,3-b]pyridin-2-yl)ethyl)- 1,2,3,6-tetrahydropyridin-4-yl)-3- methyl-N,N-bis(methyl-d₃)benzamide and (R)-4-(1-(1-(4-(6-amino-2- cyanopyridin-3-yl)-1-methyl-1H- pyrrolo[2,3-b]pyridin-2-yl)ethyl)- 1,2,3,6-tetrahydropyridin-4-yl)-3- methyl-N,N-bis(methyl-d₃)benzamide |
| 1af27 1af28 | 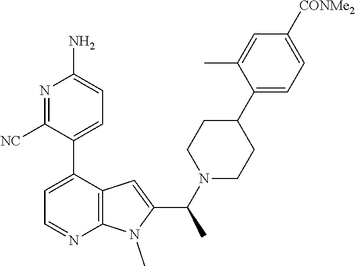 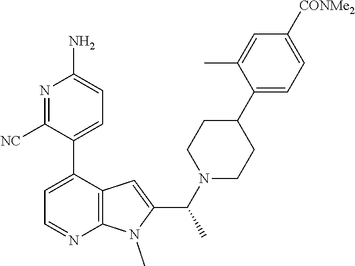 | (S)-4-(1-(1-(4-(6-Amino-2- cyanopyridin-3-yl)-1-methyl-1H- pyrrolo[2,3-b]pyridin-2- yl)ethyl)piperidin-4-yl)-N,N,3- trimethylbenzamide and (R)-4-(1-(1- (4-(6-amino-2-cyanopyridin-3-yl)-1- methyl-1H-pyrrolo[2,3-b]pyridin-2- yl)ethyl)piperidin-4-yl)-N,N,3- trimethylbenzamide |
| 1af29 1af30 | 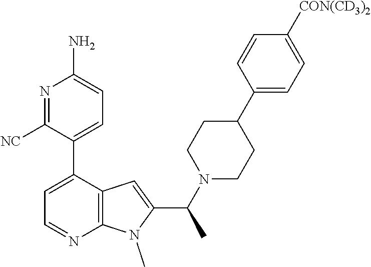 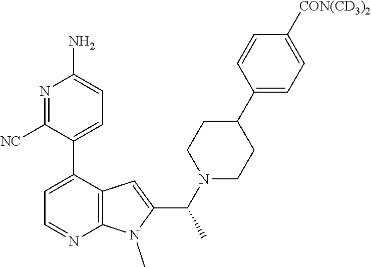 | (S)-4-(1-(1-(4-(6-Amino-2- cyanopyridin-3-yl)-1-methyl-1H- pyrrolo[2,3-b]pyridin-2-yl)ethyl)- piperidin-4-yl)-N,N-bis(methyl- d₃)benzamide and (R)-4-(1-(1-(4-(6- amino-2-cyanopyridin-3-yl)-1-methyl- 1H-pyrrolo[2,3-b]pyridin-2- yl)ethyl)piperidin-4-yl)-N,N- bis(methyl-d₃)benzamide |
| 1af31 1af33 | 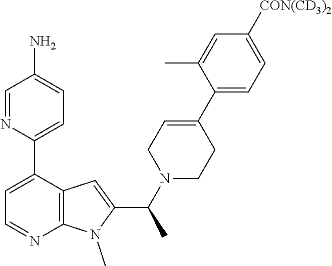 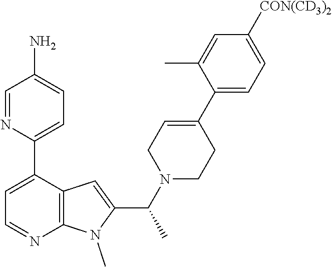 | (R)-4-(1-(1-(4-(5-aminopyridin-2-yl)- 1-methyl-1H-pyrrolo[2,3-b]pyridin-2- yl)ethyl)-1,2,3,6-tetrahydropyridin-4- yl)-3-methyl-N,N-bis(methyl- d₃)benzamide and (S)-4-(1-(1-(4-(5- aminopyridin-2-yl)-1-methyl-1H- pyrrolo[2,3-b]pyridin-2-yl)ethyl)- 1,2,3,6-tetrahydropyridin-4-yl)-3- methyl-N,N-bis(methyl-d₃)benzamide |
| 1af32 1af34 | 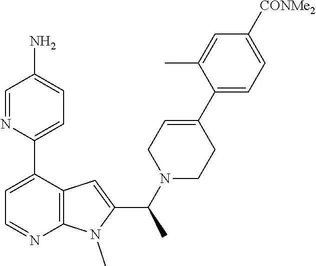 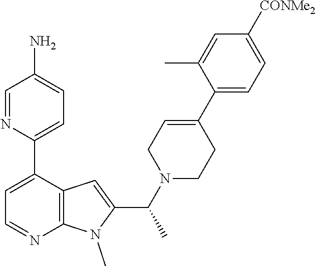 | (S)-4-(1-(1-(4-(5-Aminopyridin-2-yl)- 1-methyl-1H-pyrrolo[2,3-b]pyridin-2- yl)ethyl)-1,2,3,6-tetrahydropyridin-4- yl)-N,N,3-trimethylbenzamide and (R)- 4-(1-(1-(4-(5-aminopyridin-2-yl)-1- methyl-1H-pyrrolo[2,3-b]pyridin-2- yl)ethyl)-1,2,3,6-tetrahydropyridin-4- yl)-N,N,3-trimethylbenzamide |
| 1af35 1af36 | 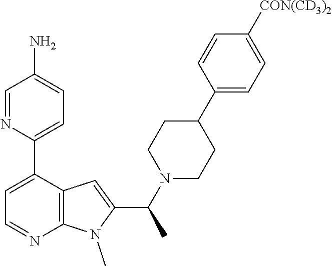 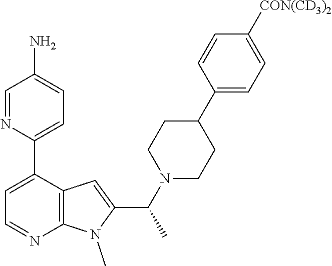 | (S)-4-(1-(1-(4-(5-Aminopyridin-2-yl)- 1-methyl-1H-pyrrolo[2,3-b]pyridin-2- yl)-ethyl)piperidin-4-yl)-N,N- bis(methyl-d₃)benzamide and (R)-4-(1- (1-(4-(5-aminopyridin-2-yl)-1-methyl- 1H-pyrrolo[2,3-b]pyridin-2- yl)ethyl)piperidin-4-yl)-N,N- bis(methyl-d₃)benzamide |
| 1af37 1af38 | 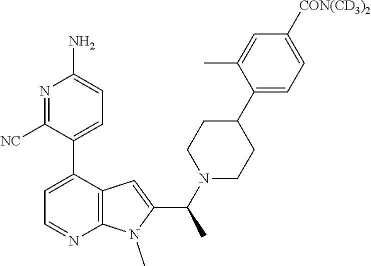 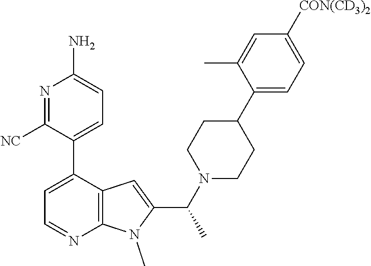 | (S)-4-(1-(1-(4-(6-Amino-2- cyanopyridin-3-yl)-1-methyl-1H- pyrrolo[2,3-b]pyridin-2- yl)ethyl)piperidin-4-yl)-3-methyl-N,N- bis(methyl-d₃)benzamide and (R)-4-(1- (1-(4-(6-amino-2-cyanopyridin-3-yl)- 1-methyl-1H-pyrrolo[2,3-b]pyridin-2- yl)ethyl)-piperidin-4-yl)-3-methyl- N,N-bis(methyl-d₃)benzamide |
| 1af39 1af40 | 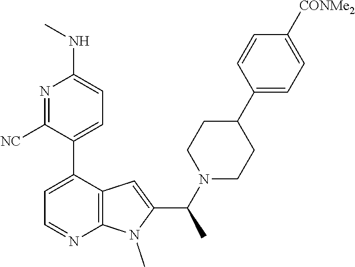 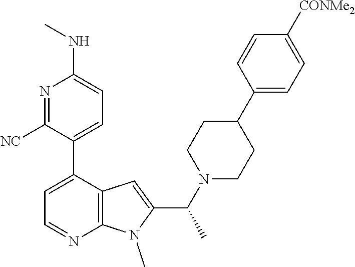 | (S)-4-(1-(1-(4-(2-xyano-6- (methylamino)pyridin-3-yl)-1-methyl- 1H-pyrrolo-[2,3-b]pyridin-2- yl)ethyl)piperidin-4-yl)-N,N- dimethylbenzamide and (R)-4-(1-(1- (4-(2-cyano-6-(methyl-amino)pyridin- 3-yl)-1-methyl-1H-pyrrolo[2,3- b]pyridin-2-yl)ethyl)piperidin-4-yl)- N,N-dimethylbenzamide |
| 1af41 1af42 | 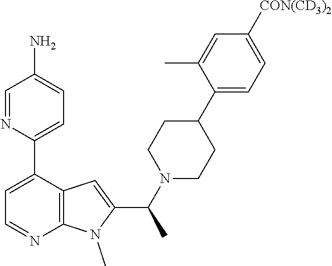 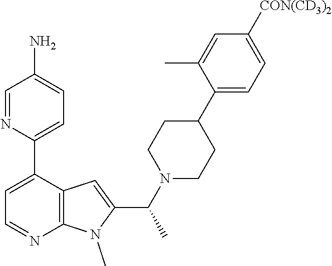 | (S)-4-(1-(1-(4-(5-Aminopyridin-2-yl)- 1-methyl-1H-pyrrolo[2,3-b]pyridin-2- yl)ethyl)-piperidin-4-yl)-3-methyl- N,N-bis(methyl-d₃)benzamide and (R)- 4-(1-(1-(4-(5-amino-pyridin-2-yl)-1- methyl-1H-pyrrolo[2,3-b]pyridin-2- yl)ethyl)piperidin-4-yl)-3-methyl-N,N- bis(methyl-d₃)benzamide |
| 1af43 1af46 | 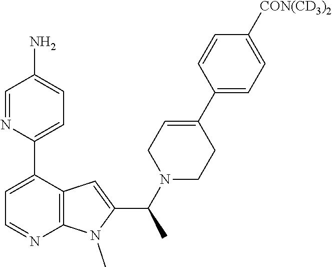 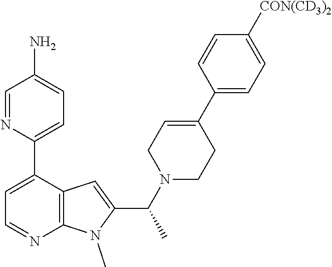 | (S)-4-(1-(1-(4-(5-Aminopyridin-2-yl)- 1-methyl-1H-pyrrolo[2,3-b]pyridin-2- yl)ethyl)-1,2,3,6-tetrahydropyridin-4- yl)-N,N-bis(methyl-d₃)benzamide and (R)-4-(1-(1-(4-(5-amino-pyridin-2-yl)- 1-methyl-1H-pyrrolo[2,3-b]pyridin-2- yl)ethyl)-1,2,3,6-tetrahydro-pyridin-4- yl)-N,N-bis(methyl-d₃)benzamide |
| 1af44 1af45 | 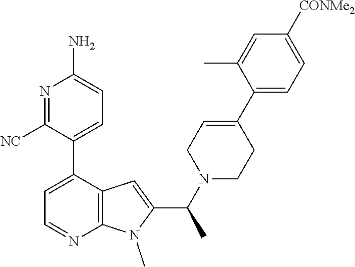 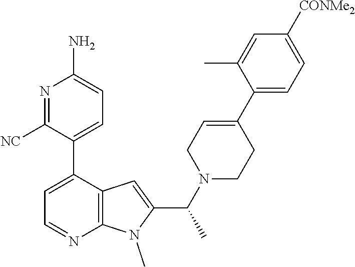 | (S)-4-(1-(1-(4-(6-Amino-2- cyanopyridin-3-yl)-1-methyl-1H- pyrrolo[2,3-b]-pyridin-2-yl)ethyl)- 1,2,3,6-tetrahydropyridin-4-yl)-N,N,3- trimethylbenzamide and (R)-4-(1-(1- (4-(6-amino-2-cyanopyridin-3-yl)-1- methyl-1H-pyrrolo[2,3-b]pyridin-2- yl)ethyl)-1,2,3,6-tetrahydropyridin-4- yl)-N,N,3-trimethylbenzamide |
| 1af50 | 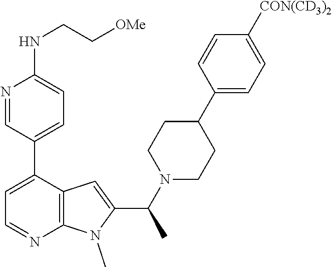 | (S)-4-(1-(1-(4-(6-((2- methoxyethyl)amino)pyridin-3-yl)-1- methyl-1H-pyrrolo[2,3-b]pyridin-2- yl)ethyl)piperidin-4-yl)-N,N- bis(methyl-d₃)benzamide |
| 1af51 | 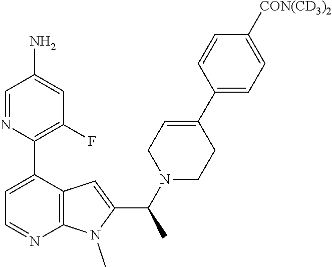 | (S)-4-(1-(1-(4-(5-Amino-3- fluoropyridin-2-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af52 | 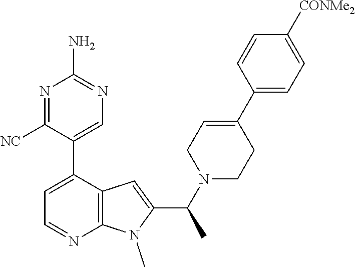 | (S)-4-(1-(1-(4-(2-Amino-4- cyanopyrimidin-5-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af53 | 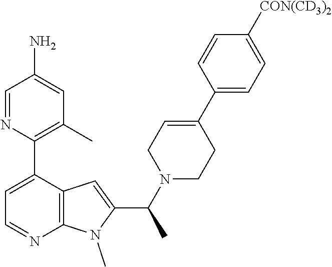 | (S)-4-(1-(1-(4-(5-Amino-3- methylpyridin-2-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af54 | 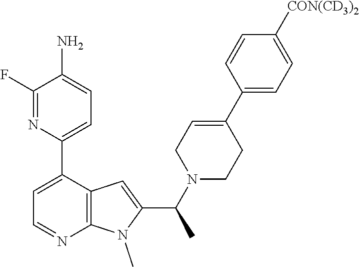 | (S)-4-(1-(1-(4-(5-Amino-6- fluoropyridin-2-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af55 | 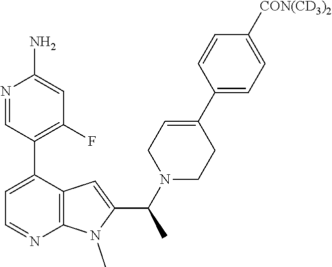 | (S)-4-(1-(1-(4-(6-Amino-4- fluoropyridin-3-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af56 | 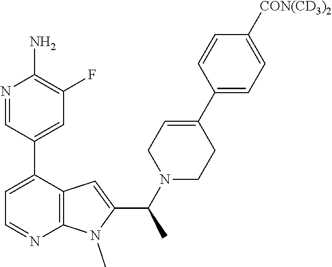 | (S)-4-(1-(1-(4-(6-Amino-5- fluoropyridin-3-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af57 | 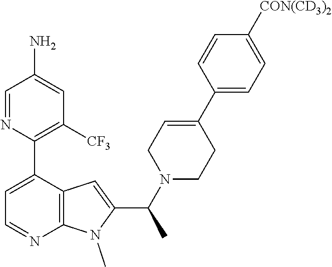 | (S)-4-(1-(1-(4-(5-Amino-3- (trifluoromethyl)pyridin-2-yl)-1- methyl-1H-pyrrolo[2,3-b]pyridin-2- yl)ethyl)-1,2,3,6-tetrahydropyridin-4- yl)-N,N-bis(methyl-d₃)benzamide |
| 1af58 | 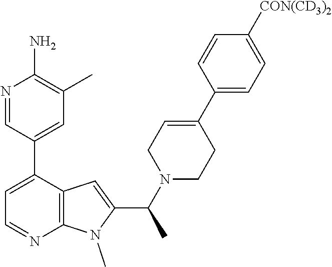 | (S)-4-(1-(1-(4-(6-Amino-5- methylpyridin-3-yl)-1-methyl-1H- pyrrolo[2,3-b]pyridin-2-yl)ethyl)- 1,2,3,6-tetrahydropyridin-4-yl)-N,N- bis(methyl-d₃)benzamide |
| 1af59 | 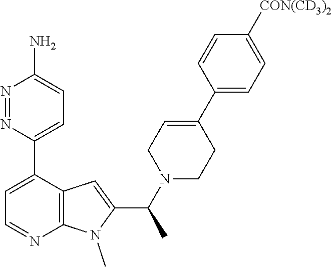 | (S)-4-(1-(1-(4-(6-aminopyridazin-3- yl)-1-methyl-1H-pyrrolo[2,3- b]pyridin-2-yl)ethyl)-1,2,3,6- tetrahydropyridin-4-yl)-N,N- bis(methyl-d₃)benzamide |
| 1af60 | 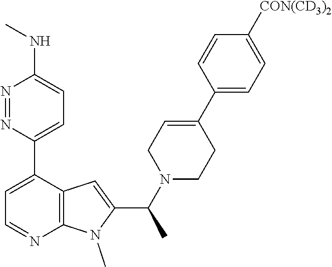 | (S)-N,N-bis(methyl-d₃)-4-(1-(1-(1- methyl-4-(6-(methylamino)pyridazin- 3-yl)-1H-pyrrolo[2,3-b]pyridin-2- yl)ethyl)-1,2,3,6-tetrahydropyridin-4- yl)benzamide |
| 1af64 | 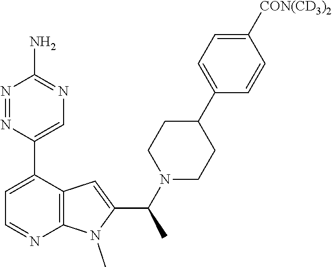 | (S)-4-(1-(1-(4-(3-amino-1,2,4-triazin- 6-yl)-1-methyl-1H-pyrrolo[2,3- b]pyridin-2-yl)ethyl)piperidin-4-yl)- N,N-bis(methyl-d₃)benzamide |
| 1af66 | 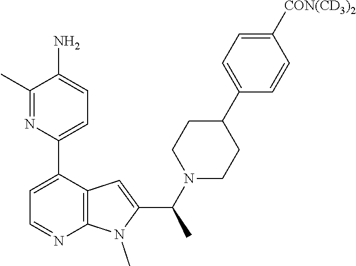 | (S)-4-(1-(1-(4-(5-amino-6- methylpyridin-2-yl)-1-methyl-1H- pyrrolo[2,3-b]pyridin-2- yl)ethyl)piperidin-4-yl)-N,N- bis(methyl-d₃)benzamide |
| 1af67 | 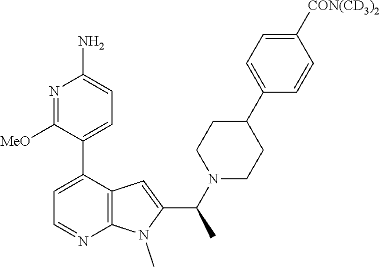 | (S)-4-(1-(1-(4-(6-amino-2- methoxypyridin-3-yl)-1-methyl-1H- pyrrolo[2,3-b]pyridin-2- yl)ethyl)piperidin-4-yl)-N,N- bis(methyl-d₃)benzamide |
| 1af68 | 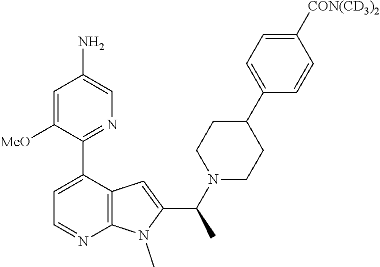 | (S)-4-(1-(1-(4-(5-amino-3- methoxypyridin-2-yl)-1-methyl-1H- pyrrolo[2,3-b]pyridin-2- yl)ethyl)piperidin-4-yl)-N,N- bis(methyl-d₃)benzamide |
| 1af69 | 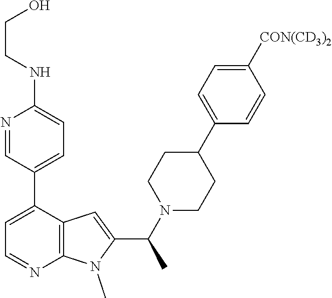 | (S)-4-(1-(1-(4-(6-((2- hydroxyethyl)amino)pyridin-3-yl)-1- methyl-1H-pyrrolo[2,3-b]pyridin-2- yl)ethyl)piperidin-4-yl)-N,N- bis(methyl-d₃)benzamide |
| 1af70 | 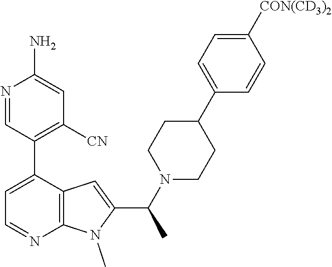 | (S)-4-(1-(1-(4-(6-amino-4- cyanopyridin-3-yl)-1-methyl-1H- pyrrolo[2,3-b]pyridin-2- yl)ethyl)piperidin-4-yl)-N,N- bis(methyl-d₃)benzamide |
| 1af72 | 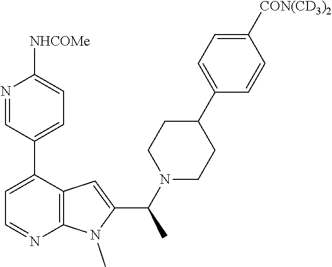 | 4-((2R)-1-((4-(6-acetamidopyridin-3- yl)-1-methyl-1H-pyrrolo[2,3- b]pyridin-2-yl)methyl)-2- methylpiperidin-4-yl)-N,N-bis(methyl- d₃)benzamide |
| 1af73 | 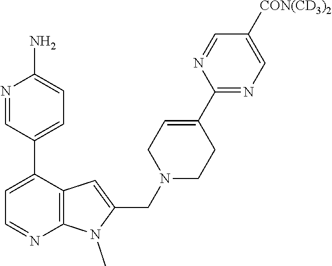 | 2-(1-((4-(6-Aminopyridin-3-yl)-1- methyl-1H-pyrrolo[2,3-b]pyridin-2- yl)methyl)-1,2,3,6-tetrahydropyridin- 4-yl)-N,N-bis(methyl-d₃)pyrimidine-5- carboxamide |
| 1af75 | 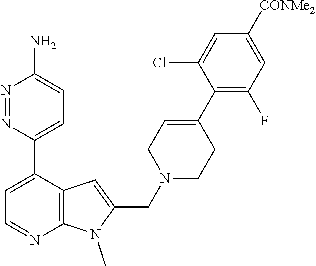 | 4-(1-((4-(6-Aminopyridazin-3-yl)-1- methyl-1H-pyrrolo[2,3-b]pyridin-2- yl)methyl)-1,2,3,6-tetrahydropyridin- 4-yl)-3-chloro-5-fluoro-N,N- dimethylbenzamide |
| 1af76 | 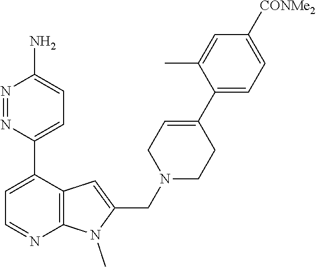 | 4-(1-((4-(6-Aminopyridazin-3-yl)-1- methyl-1H-pyrrolo[2,3-b]pyridin-2- yl)methyl)-1,2,3,6-tetrahydropyridin- 4-yl)-N,N,3-trimethylbenzamide |
| 1af77 | 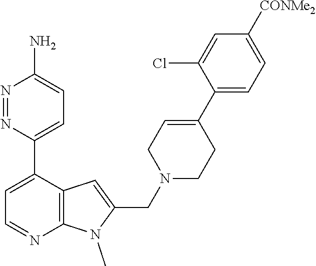 | 4-(1-((4-(6-Aminopyridazin-3-yl)-1- methyl-1H-pyrrolo[2,3-b]pyridin-2- yl)methyl)-1,2,3,6-tetrahydropyridin- 4-yl)-3-chloro-N,N- dimethylbenzamide |
| 1af78 | 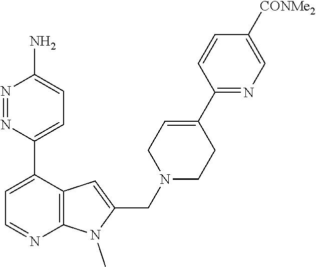 | 1′-((4-(6-Aminopyridazin-3-yl)-1- methyl-1H-pyrrolo[2,3-b]pyridin-2- yl)methyl)-N,N-dimethyl-1′,2′,3′,6′- tetrahydro-[2,4′-bipyridine]-5- carboxamide |
| 1af79 | 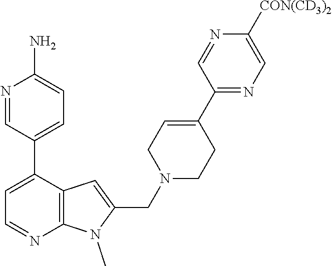 | 5-(1-((4-(6-Aminopyridin-3-yl)-1- methyl-1H-pyrrolo[2,3-b]pyridin-2- yl)methyl)-1,2,3,6-tetrahydropyridin- 4-yl)-N,N-bis(methyl-d₃)pyrazine-2- carboxamide |
| 1af80 | 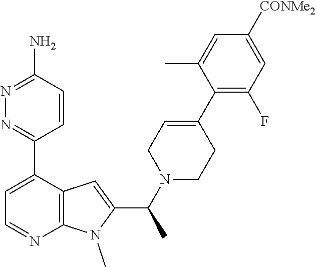 | (S)-4-(1-(1-(4-(6-aminopyridazin-3- yl)-1-methyl-1H-pyrrolo[2,3- b]pyridin-2-yl)ethyl)-1,2,3,6- tetrahydropyridin-4-yl)-3-fluoro- N,N,5-trimethylbenzamide |
| 1af81 | 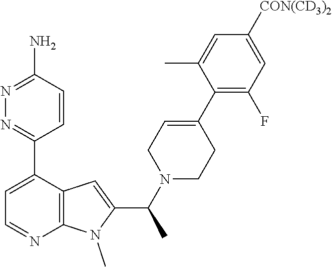 | (S)-4-(1-(1-(4-(6-aminopyridazin-3- yl)-1-methyl-1H-pyrrolo[2,3- b]pyridin-2-yl)ethyl)-1,2,3,6- tetrahydropyridin-4-yl)-3-fluoro-5- methyl-N,N-bis(methyl-d₃)benzamide |
| 1j6 | 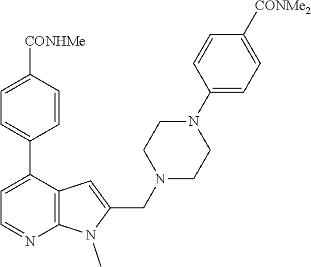 | 4-[2-[[4-[4- (Dimethylcarbamoyl)phenyl]piperazin- 1-yl]methyl]-1-methyl-pyrrolo[2,3- b]pyridin-4-yl]-N-methyl-benzamide |
| 1j7 | 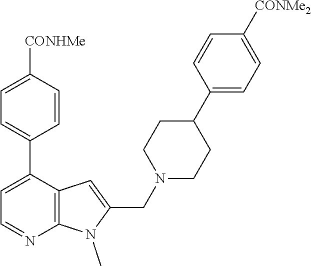 | 4-[2-[[4-[4- (Dimethylcarbamoyl)phenyl]-1- piperidyl]methyl]-1-methyl- pyrrolo[2,3-b]pyridin-4-yl]-N-methyl- benzamide |
| 1j13 | 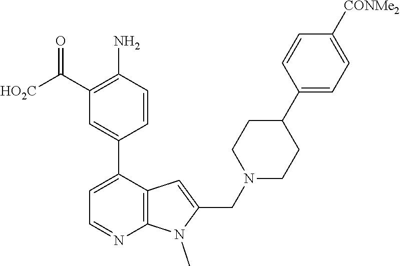 | 2-(2-Amino-5-(2-((4-(4- (dimethylcarbamoyl)phenyl)piperidin- 1-yl)methyl)-1-methyl-1H-pyrrolo[2,3- b]pyridin-4-yl)phenyl)-2-oxoacetic acid |
| 1k2 | 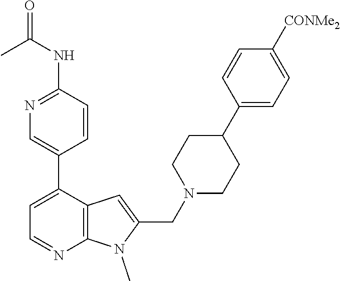 | 4-[1-[[4-(6-Acetamido-3-pyridyl)-1- methyl-pyrrolo[2,3-b]pyridin-2- yl]methyl]-4-piperidyl]-N,N-dimethyl- benzamide |
| 1k3 | 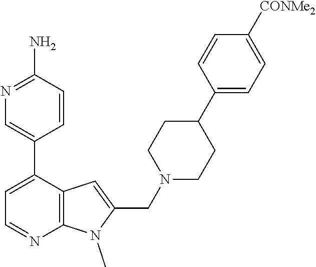 | 4-[1-[[4-(6-Amino-3-pyridyl)-1- methyl-pyrrolo[2,3-b]pyridin-2- yl]methyl]-4-piperidyl]-N,N-dimethyl- benzamide |
| 1k6 | 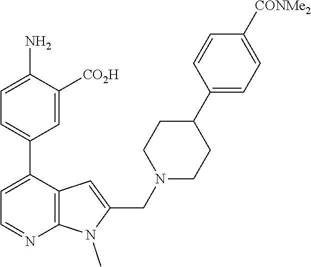 | 2-Amino-5-[2-[[4-[4- (dimethylcarbamoyl)phenyl]-1- piperidyl]methyl]-1-methyl- pyrrolo[2,3-b]pyridin-4-yl]benzoic acid |
| 1k7 | 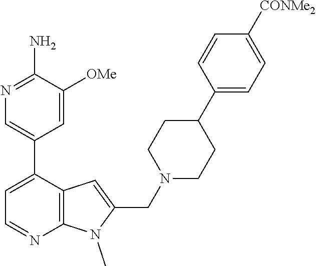 | 4-(1-((4-(6-Amino-5-methoxypyridin- 3-yl)-1-methyl-1H-pyrrolo[2,3- b]pyridin-2-yl)methyl)piperidin-4-yl)- N,N-dimethylbenzamide |
| 1k8 | 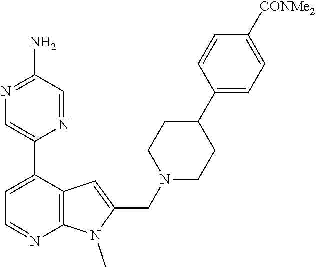 | 4-[1-[[4-(5-Aminopyrazin-2-yl)-1- methyl-pyrrolo[2,3-b]pyridin-2- yl]methyl]-4-piperidyl]-N,N-dimethyl- benzamide |
| 1k9 | 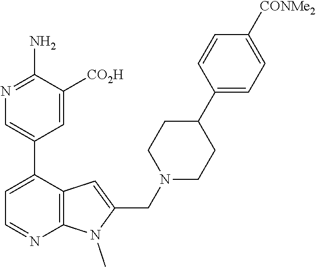 | 2-Amino-5-[2-[[4-[4- (dimethylcarbamoyl)phenyl]-1- piperidyl]methyl]-1-methyl- pyrrolo[2,3-b]pyridin-4-yl]pyridine-3- carboxylic acid |
| 1k10 | 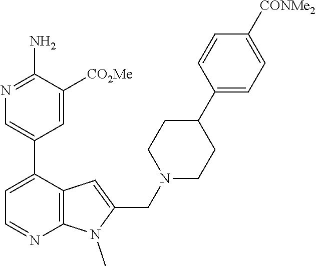 | Methyl 2-amino-5-[2-[[4-[4- (dimethylcarbamoyl)phenyl]-1- piperidyl]methyl]-1-methyl- pyrrolo[2,3-b]pyridin-4-yl]pyridine-3- carboxylate |
| 1k11 | 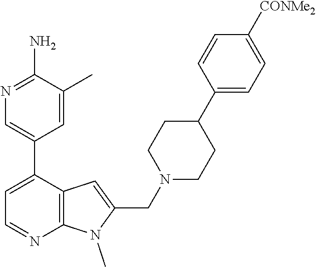 | 4-[1-[[4-(6-Amino-5-methyl-3- pyridyl)-1-methyl-pyrrolo[2,3- b]pyridin-2-yl]methyl]-4-piperidyl]- N,N-dimethyl-benzamide |
| 1k12 | 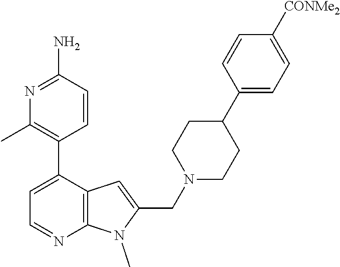 | 4-[1-[[4-(6-Amino-2-methyl-3- pyridyl)-1-methyl-pyrrolo[2,3- b]pyridin-2-yl]methyl]-4-piperidyl]- N,N-dimethyl-benzamide |
| 1k13 | 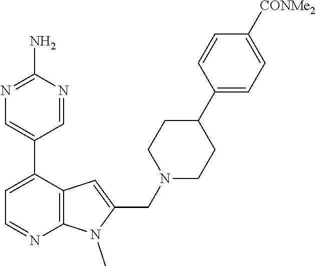 | 4-[1-[[4-(2-Aminopyrimidin-5-yl)-1- methyl-pyrrolo[2,3-b]pyridin-2- yl]methyl]-4-piperidyl]-N,N-dimethyl- benzamide |
| 1k14 | 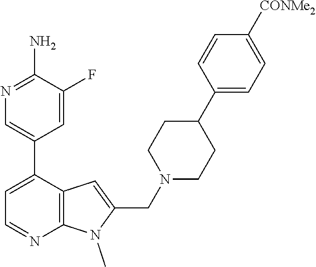 | 4-[1-[[4-(6-Amino-5-fluoro-3-pyridyl)- 1-methyl-pyrrolo[2,3-b]pyridin-2- yl]methyl]-4-piperidyl]-N,N-dimethyl- benzamide |
| 1k15 | 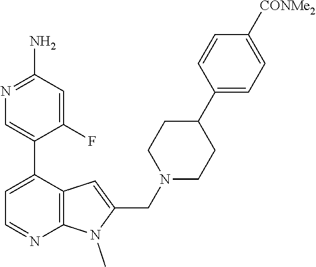 | 4-[1-[[4-(6-Amino-4-fluoro-3-pyridyl)- 1-methyl-pyrrolo[2,3-b]pyridin-2- yl]methyl]-4-piperidyl]-N,N-dimethyl- benzamide |
| 1k16 | 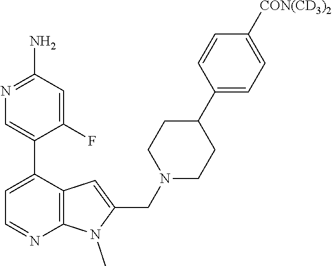 | 4-[1-[[4-(6-Amino-4-fluoro-3-pyridyl)- 1-methyl-pyrrolo[2,3-b]pyridin-2- yl]methyl]-4-piperidyl]-N,N- bis(trideuteriomethyl)benzamide |
| 1k17 | 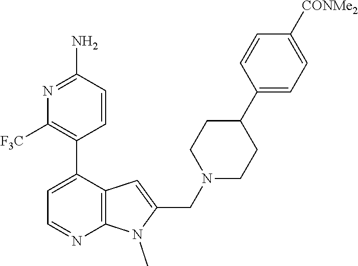 | 4-[1-[4-[6-Amino-2-(trifluoromethyl)- 3-pyridyl]-1-methyl-pyrrolo[2,3- b]pyridin-2-yl]methyl]-4-piperidyl]- N,N-dimethyl-benzamide |
| 1k18 | 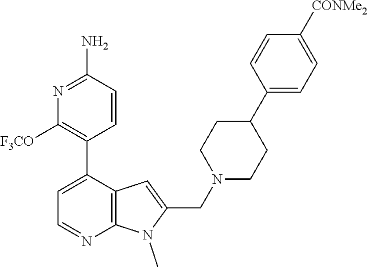 | 4-[1-[[4-[6-Amino-2- (trifluoromethoxy)-3-pyridyl]-1- methyl-pyrrolo[2,3-b]pyridin-2- yl]methyl]-4-piperidyl]-N,N-dimethyl- benzamide |
| 1k19 | 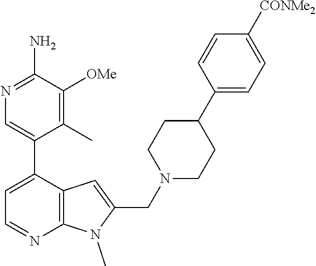 | 4-[1-[[4-[6-Amino-2- (trifluoromethoxy)-3-pyridyl]-1- methyl-pyrrolo[2,3-b]pyridin-2- yl]methyl]-4-piperidyl]-N,N-dimethyl- benzamide |
| 1k20 | 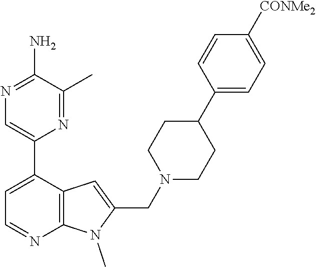 | 4-[1-[[4-(5-Amino-6-methyl-pyrazin- 2-yl)-1-methyl-pyrrolo[2,3-b]pyridin- 2-yl]methyl]-4-piperidyl]-N,N- dimethyl-benzamide |
| 1k21 | 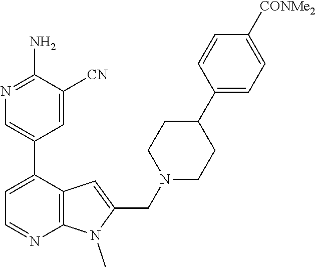 | 4-[1-[[4-(6-Amino-5-cyano-3-pyridyl)- 1-methyl-pyrrolo[2,3-b]pyridin-2- yl]methyl]-4-piperidyl]-N,N-dimethyl- benzamide |
| 1k22 | 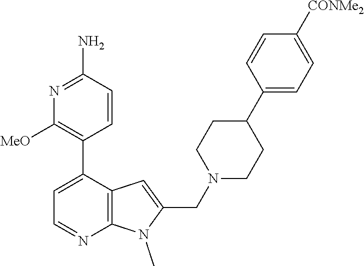 | 4-[1-[[4-(6-Amino-2-methoxy-3- pyridyl)-1-methyl-pyrrolo[2,3- b]pyridin-2-yl]methyl]-4-piperidyl]- N,N-dimethyl-benzamide |
| 1k23 | 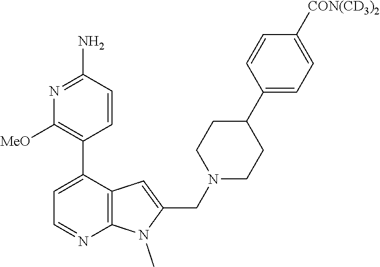 | 4-[1-[[4-(6-Amino-2-methoxy-3- pyridyl)-1-methyl-pyrrolo[2,3- b]pyridin-2-yl]methyl]-4-piperidyl]- N,N-bis(trideuteriomethyl)benzamide |
| 1k24 | 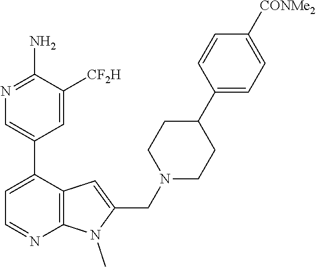 | 4-[1-[4-[6-Amino-5-(difluoromethyl)- 3-pyridyl]-1-methyl-pyrrolo[2,3- b]pyridin-2-yl]methyl]-4-piperidyl]- N,N-dimethyl-benzamide |
| 1k25 | 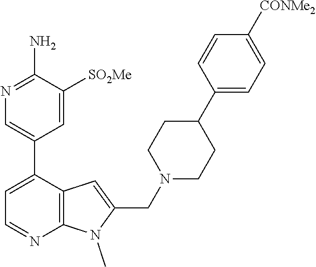 | 4-[1-[[4-(6-Amino-5-methylsulfonyl- 3-pyridyl)-1-methyl-pyrrolo[2,3- b]pyridin-2-yl]methyl]-4-piperidyl]- N,N-dimethyl-benzamide |
| 1k26 | 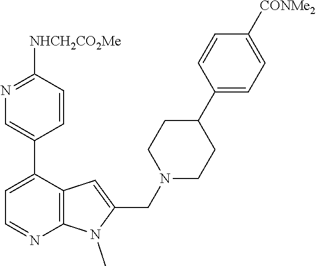 | Methyl 2-[[5-[2-[[4-[4- (dimethylcarbamoyl)phenyl]-1- piperidyl]methyl]-1-methyl- pyrrolo[2,3-b]pyridin-4-yl]-2- pyridyl]amino]acetate |
| 1k27 | 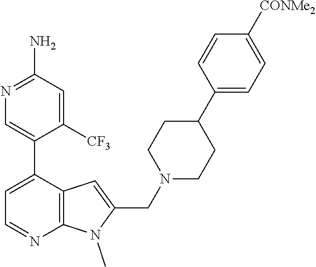 | 4-[1-[[4-[6-Amino-4-(trifluoromethyl)- 3-pyridyl]-1-methyl-pyrrolo[2,3- b]pyridin-2-yl]methyl]-4-piperidyl]- N,N-dimethyl-benzamide |
| 1k28 | 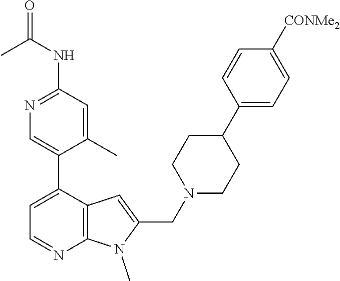 | 4-[1-[[4-(6-Acetamido-4-methyl-3- pyridyl)-1-methyl-pyrrolo[2,3- b]pyridin-2-yl]methyl]-4-piperidyl]- N,N-dimethyl-benzamide |
| 1k30 | 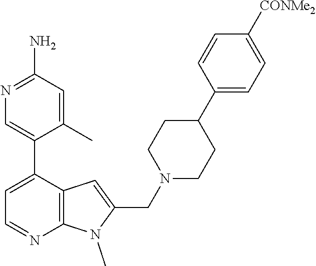 | 4-[1-[[4-(6-Amino-4-methyl-3- pyridyl)-1-methyl-pyrrolo[2,3- b]pyridin-2-yl]methyl]-4-piperidyl]- N,N-dimethyl-benzamide |
| 1k31 | 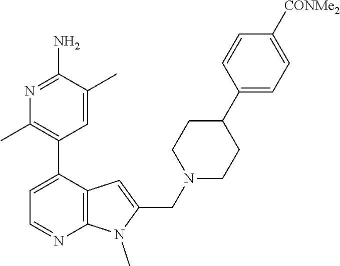 | 4-[1-[[4-(6-Amino-2,5-dimethyl-3- pyridyl)-1-methyl-pyrrolo[2,3- b]pyridin-2-yl]methyl]-4-piperidyl]- N,N-dimethyl-benzamide |
| 1k32 | 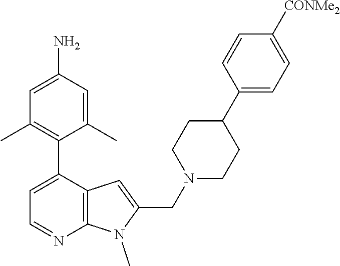 | 4-[1-[[4-(4-Amino-2,6-dimethyl- phenyl)-1-methyl-pyrrolo[2,3- b]pyridin-2-yl]methyl]-4-piperidyl]- N,N-dimethyl-benzamide |
| 1k33 | 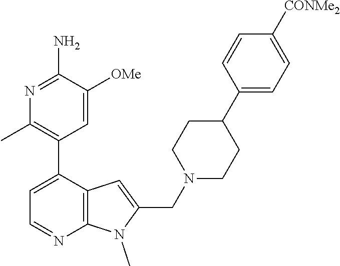 | 4-[1-[[4-(6-Amino-5-methoxy-2- methyl-3-pyridyl)-1-methyl- pyrrolo[2,3-b]pyridin-2-yl]methyl]-4- piperidyl]-N,N-dimethyl-benzamide |
| 1k34 | 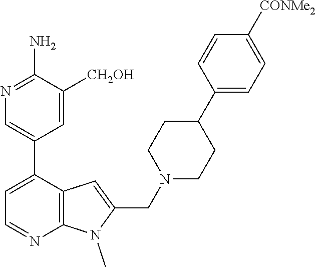 | 4-[1-[[4-[6-Amino-5-(hydroxymethyl)- 3-pyridyl]-1-methyl-pyrrolo[2,3- b]pyridin-2-yl]methyl]-4-piperidyl]- N,N-dimethyl-benzamide |
| 1k35 | 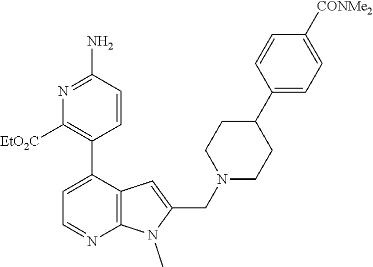 | Ethyl 6-amino-3-[2-[[4-[4- (dimethylcarbamoyl)phenyl]-1- piperidyl]methyl]-1-methyl- pyrrolo[2,3-b]pyridin-4-yl]pyridine-2- carboxylate |
| 1k36 | 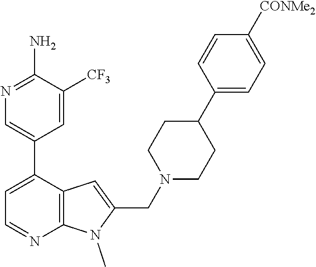 | 4-[1-[[4-[6-Amino-5-(trifluoromethyl)- 3-pyridyl]-1-methyl-pyrrolo[2,3- b]pyridin-2-yl]methyl]-4-piperidyl]- N,N-dimethyl-benzamide |
| 1k37 | 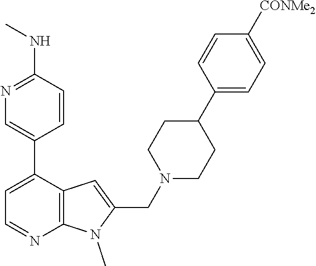 | N,N-Dimethyl-4-[1-[1-methyl-4-[6- (methylamino)-3-pyridyl]pyrrolo[2,3- b]pyridin-2-yl]methyl]-4- piperidyl]benzamide |
| 1k38 | 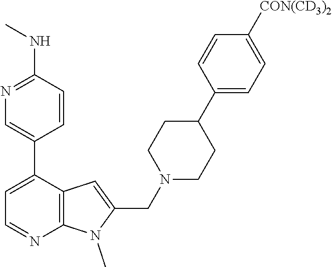 | 4-[1-[[1-Methyl-4-[6-(methylamino)- 3-pyridyl]pyrrolo[2,3-b]pyridin-2- yl]methyl]-4-piperidyl]-N,N- bis(trideuteriomethyl)benzamide |
| 1k39 | 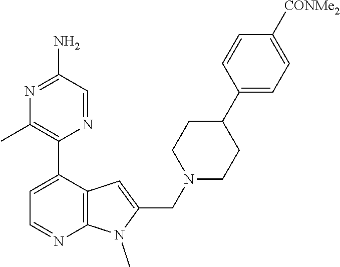 | 4-[1-[[4-(5-Amino-3-methyl-pyrazin- 2-yl)-1-methyl-pyrrolo[2,3-b]pyridin- 2-yl]methyl]-4-piperidyl]-N,N- dimethyl-benzamide |
| 1k40 | 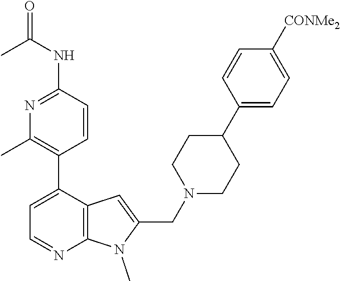 | 4-[1-[[4-(6-Acetamido-2-methyl-3- pyridyl)-1-methyl-pyrrolo[2,3- b]pyridin-2-yl]methyl]-4-piperidyl]- N,N-dimethyl-benzamide |
| 1k41 | 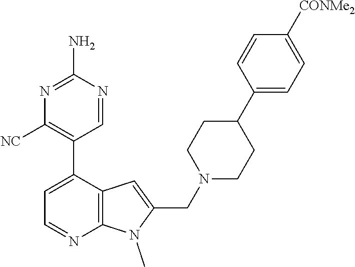 | 4-[1-[[4-(2-Amino-4-cyano-pyrimidin- 5-yl)-1-methyl-pyrrolo[2,3-b]pyridin- 2-yl]methyl]-4-piperidyl]-N,N- dimethyl-benzamide |
| 1k42 | 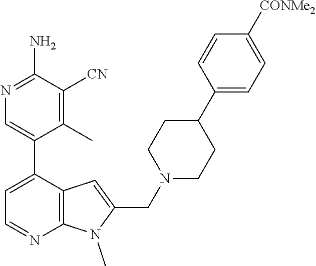 | 4-[1-[[4-(6-Amino-5-cyano-4-methyl- 3-pyridyl)-1-methyl-pyrrolo[2,3- b]pyridin-2-yl]methyl]-4-piperidyl]- N,N-dimethyl-benzamide |
| 1k43 | 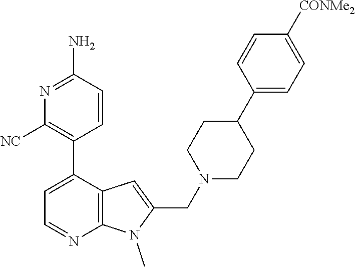 | 4-[1-[[4-(6-Amino-2-cyano-3-pyridyl)- 1-methyl-pyrrolo[2,3-b]pyridin-2- yl]methyl]-4-piperidyl]-N,N-dimethyl- benzamide |
| 1k44 | 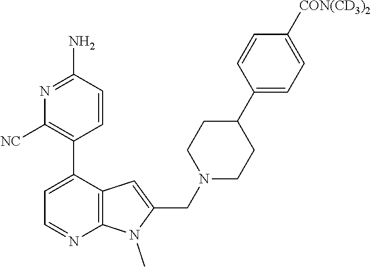 | 4-[1-[[4-(6-Amino-2-methoxy-3- pyridyl)-1-methyl-pyrrolo[2,3- b]pyridin-2-yl]methyl]-4-piperidyl]- N,N-bis(trideuteriomethyl)benzamide |
| 1k45 | 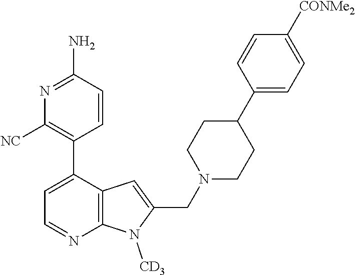 | 4-[1-[[4-(6-Amino-2-cyano-3-pyridyl)- 1-(trideuteriomethyl)pyrrolo[2,3- b]pyridin-2-yl]methyl]-4-piperidyl]- N,N-dimethyl-benzamide |
| 1k47 | 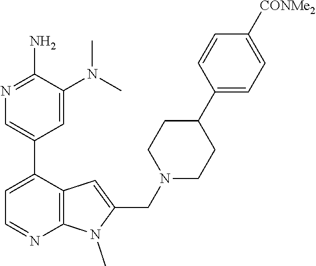 | 4-[1-[[4-[6-Amino-5-(dimethylamino)- 3-pyridyl]-1-methyl-pyrrolo[2,3- b]pyridin-2-yl]methyl]-4-piperidyl]- N,N-dimethyl-benzamide |
| 1k48 | 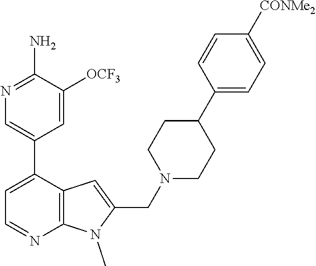 | 4-[1-[[4-[6-Amino-5- (trifluoromethoxy)-3-pyridyl]-1- methyl-pyrrolo[2,3-b]pyridin-2- yl]methyl]-4-piperidyl]-N,N-dimethyl- benzamide |
| 1k49 | 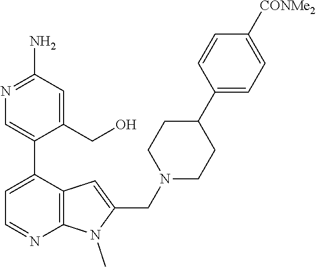 | 4-[1-[[4-[6-Amino-4-(hydroxymethyl)- 3-pyridyl]-1-methyl-pyrrolo[2,3- b]pyridin-2-yl]methyl]-4-piperidyl]- N,N-dimethyl-benzamide |
| 1k50 | 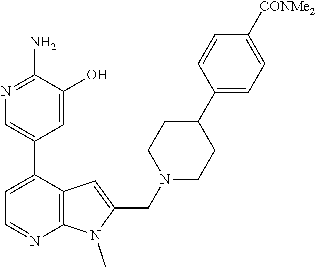 | 4-[1-[[4-(6-Amino-5-hydroxy-3- pyridyl)-1-methyl-pyrrolo[2,3- b]pyridin-2-yl]methyl]-4-piperidyl]- N,N-dimethyl-benzamide |
| 1k51 | 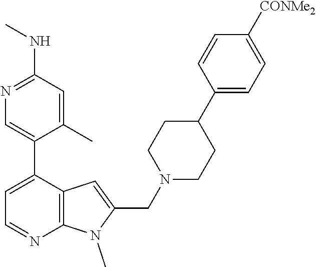 | N,N-Dimethyl-4-[1-[[1-methyl-4-[4- methyl-6-(methylamino)-3- pyridyl]pyrrolo[2,3-b]pyridin-2- yl]methyl]-4-piperidyl]benzamide |
| 1k52 | 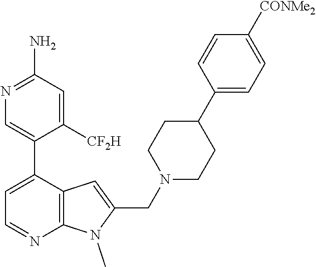 | 4-[1-[[4-[6-Amino-4-(difluoromethyl)- 3-pyridyl]-1-methyl-pyrrolo[2,3- b]pyridin-2-yl]methyl]-4-piperidyl]- N,N-dimethyl-benzamide |
| 1k53 | 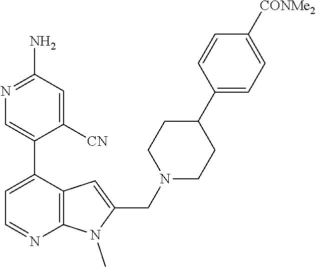 | 4-[1-[[4-(6-Amino-4-cyano-3-pyridyl)- 1-methyl-pyrrolo[2,3-b]pyridin-2- yl]methyl]-4-piperidyl]-N,N-dimethyl- benzamide |
| 1k54 | 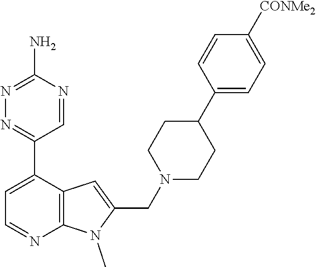 | 4-[1-[[4-(3-Amino-1,2,4-triazin-6-yl)- 1-methyl-pyrrolo[2,3-b]pyridin-2- yl]methyl]-4-piperidyl]-N,N-dimethyl- benzamide |
| 1k55 | 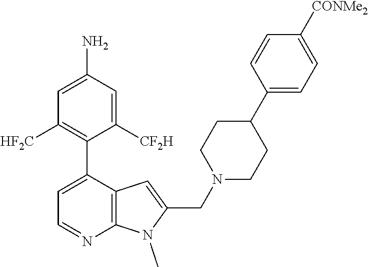 | 4-[1-[[4-[4-Amino-2,6- bis(difluoromethyl)phenyl]-1-methyl- pyrrolo[2,3-b]pyridin-2-yl]methyl]-4- piperidyl]-N,N-dimethyl-benzamide |
| 1k56 | 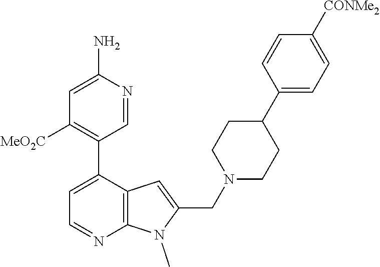 | Methyl 2-amino-5-[2-[[4-[4- (dimethylcarbamoyl)phenyl]-1- piperidyl]methyl]-1-methyl- pyrrolo[2,3-b]pyridin-4-yl]pyridine-4- carboxylate |
| 1k57 | 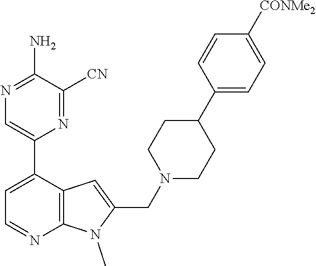 | 4-[1-[[4-(5-Amino-6-cyano-pyrazin-2- yl)-1-methyl-pyrrolo[2,3-b]pyridin-2- yl]methyl]-4-piperidyl]-N,N-dimethyl- benzamide |
| 1k58 | 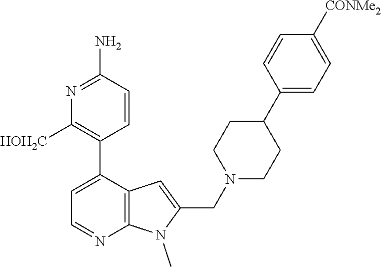 | 4-[1-[[4-[6-Amino-2-(hydroxymethyl)- 3-pyridyl]-1-methyl-pyrrolo[2,3- b]pyridin-2-yl]methyl]-4-piperidyl]- N,N-dimethyl-benzamide |
| 1k59 | 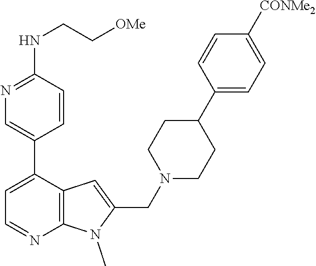 | 4-[1-[[4-[6-(2-Methoxyethylamino)-3- pyridyl]-1-methyl-pyrrolo[2,3- b]pyridin-2-yl]methyl]-4-piperidyl]- N,N-dimethyl-benzamide |
| 1k60 | 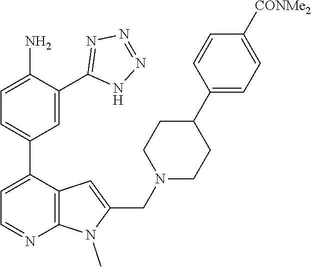 | 4-[1-[[4-[4-Amino-3-(2H-tetrazol-5- yl)phenyl]-1-methyl-pyrrolo[2,3- b]pyridin-2-yl]methyl]-4-piperidyl]- N,N-dimethyl-benzamide |
| 1k61 | 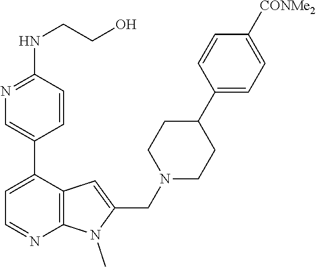 | 4-[1-[[4-[6-(2-Hydroxyethylamino)-3- pyridyl]-1-methyl-pyrrolo[2,3- b]pyridin-2-yl]methyl]-4-piperidyl]- N,N-dimethyl-benzamide |
| 1k63 | 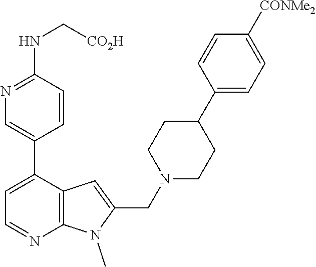 | 2-[[5-[2-[[4-[4- (Dimethylcarbamoyl)phenyl]-1- piperidyl]methyl]-1-methyl- pyrrolo[2,3-b]pyridin-4-yl]-2- pyridyl]amino]acetic acid |
| 1k64 | 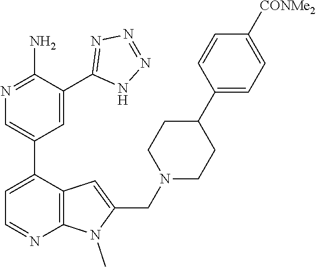 | 4-[1-[[4-[6-Amino-5-(2H-tetrazol-5- yl)-3-pyridyl]-1-methyl-pyrrolo[2,3- b]pyridin-2-yl]methyl]-4-piperidyl]- N,N-dimethyl-benzamide |
| 1k65 | 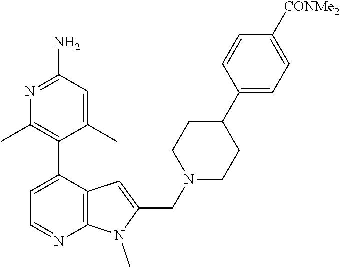 | 4-[1-[[4-(6-Amino-2,4-dimethyl-3- pyridyl)-1-methyl-pyrrolo[2,3- b]pyridin-2-yl]methyl]-4-piperidyl]- N,N-dimethyl-benzamide |
| 1k66 | 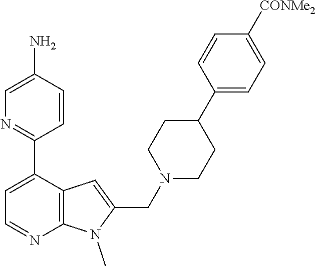 | 4-(1-((4-(5-Aminopyridin-2-yl)-1- methyl-1H-pyrrolo[2,3-b]pyridin-2- yl)methyl)piperidin-4-yl)-N,N- dimethylbenzamide |
| 1k67 | 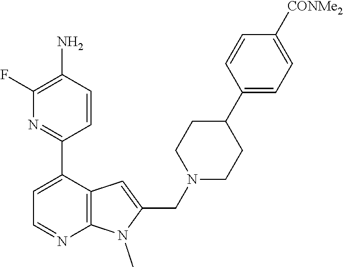 | 4-(1-((4-(5-Amino-6-fluoropyridin-2- yl)-1-methyl-1H-pyrrolo[2,3- b]pyridin-2-yl)methyl)piperidin-4-yl)- N,N-dimethylbenzamide |
| 1k68 | 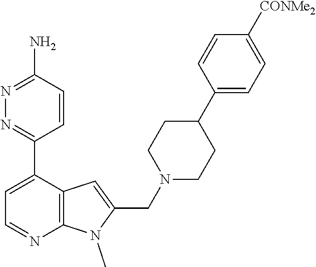 | 4-[1-[[4-(6-Aminopyridazin-3-yl)-1- methyl-pyrrolo[2,3-b]pyridin-2- yl]methyl]-4-piperidyl]-N,N-dimethyl- benzamide |
| 1k69 | 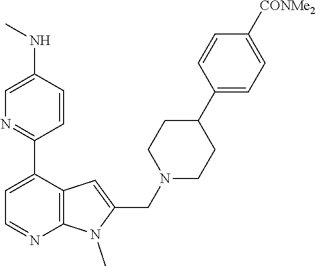 | N,N-Dimethyl-4-(1-((1-methyl-4-(5- (methylamino)pyridin-2-yl)-1H- pyrrolo[2,3-b]pyridin-2- yl)methyl)piperidin-4-yl)benzamide |
| 1k70 | 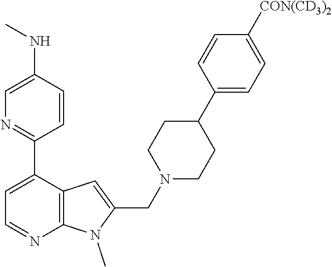 | 4-[1-[[1-methyl-4-[5-(methylamino)-2- pyridyl]pyrrolo[2,3-b]pyridin-2- yl]methyl]-4-piperidyl]-N,N- bis(trideuteriomethyl)benzamide |
| 1k71 | 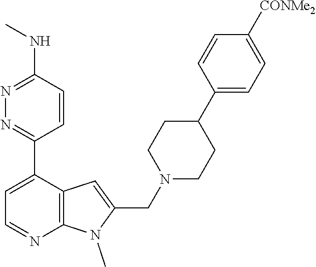 | N,N-Dimethyl-4-[1-[[1-methyl-4-[6- (methylamino)pyridazin-3- yl]pyrrolo[2,3-b]pyridin-2-yl]methyl]- 4-piperidyl]benzamide |
| 1k72 | 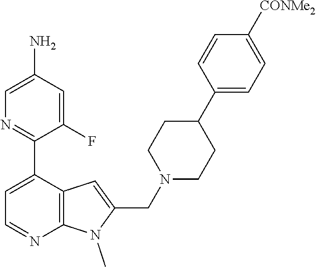 | 4-[1-[[4-(5-Amino-3-fluoro-2-pyridyl)- 1-methyl-pyrrolo[2,3-b]pyridin-2- yl]methyl]-4-piperidyl]-N,N-dimethyl- benzamide |
| 1k73 | 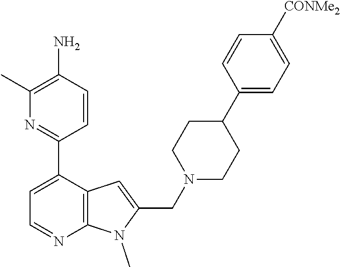 | 4-[1-[[4-(5-Amino-6-methyl-2- pyridyl)-1-methyl-pyrrolo[2,3- b]pyridin-2-yl]methyl]-4-piperidyl]- N,N-dimethyl-benzamide |
| 1k74 | 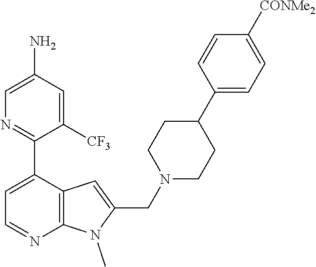 | 4-[1-[[4-[5-Amino-3-(trifluoromethyl)- 2-pyridyl]-1-methyl-pyrrolo[2,3- b]pyridin-2-yl]methyl]-4-piperidyl]- N,N-dimethyl-benzamide |
| 1k75 | 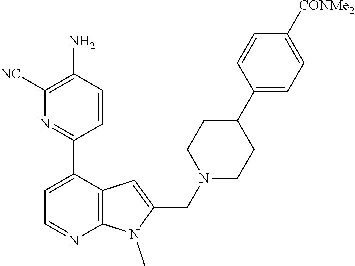 | 4-(1-((4-(5-Amino-6-cyanopyridin-2- yl)-1-methyl-1H-pyrrolo[2,3- b]pyridin-2-yl)methyl)piperidin-4-yl)- N,N-dimethylbenzamide |
| 1k76 | 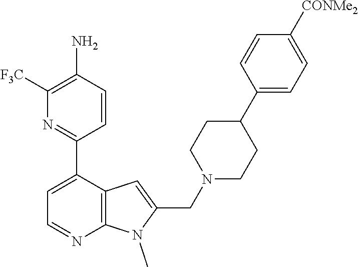 | 4-[1-[[4-[5-Amino-6-(trifluoromethyl)- 2-pyridyl]-1-methyl-pyrrolo[2,3- b]pyridin-2-yl]methyl]-4-piperidyl]- N,N-dimethyl-benzamide |
| 1k77 | 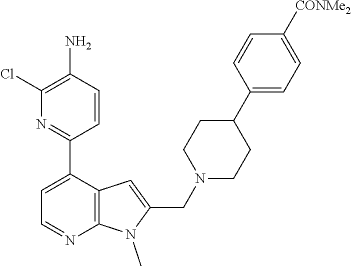 | 4-(1-((4-(5-Amino-6-chloropyridin-2- yl)-1-methyl-1H-pyrrolo[2,3- b]pyridin-2-yl)methyl)piperidin-4-yl)- N,N-dimethylbenzamide |
| 1k78 | 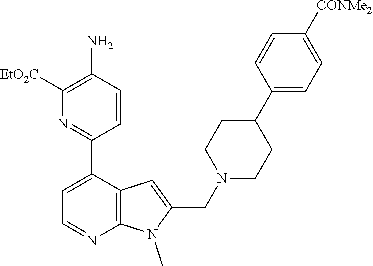 | Ethyl 3-amino-6-(2-((4-(4- (dimethylcarbamoyl)phenyl)piperidin- 1-yl)methyl)-1-methyl-1H-pyrrolo[2,3- b]pyridin-4-yl)picolinate |
| 1k79 | 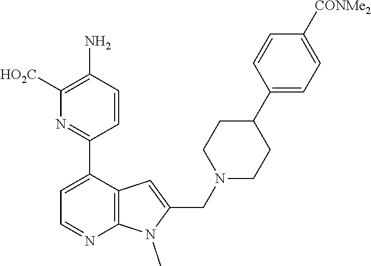 | 3-Amino-6-[2-[[4-[4- (dimethylcarbamoyl)phenyl]-1- piperidyl]methyl]-1-methyl- pyrrolo[2,3-b]pyridin-4-yl]pyridine-2- carboxylic acid |
| 1k80 | 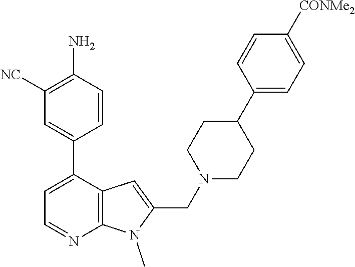 | 4-(1-((4-(4-Amino-3-cyanophenyl)-1- methyl-1H-pyrrolo[2,3-b]pyridin-2- yl)methyl)piperidin-4-yl)-N,N- dimethylbenzamide |
| 1m145 | 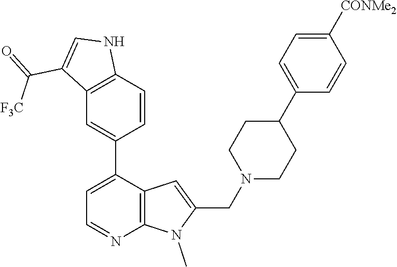 | N,N-dimethyl-4-(1-((1-methyl-4-(3- (2,2,2-trifluoroacetyl)-1H-indol-5-yl)- 1H-pyrrolo[2,3-b]pyridin-2- yl)methyl)piperidin-4-yl)benzamide |
| 1n4 | 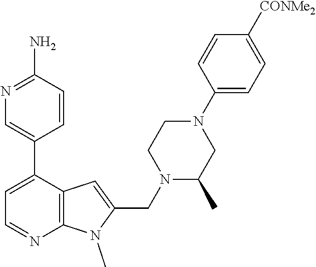 | 4-[(3R)-4-[[4-(6-amino-3-pyridyl)-1- methyl-pyrrolo[2,3-b]pyridin-2- yl]methyl]-3-methyl-piperazin-1-yl]- N,N-dimethyl-benzamide |
| 1n5 | 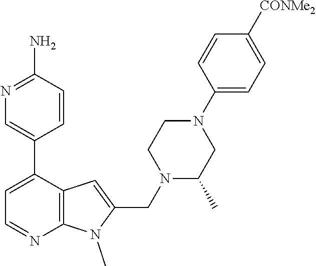 | 4-[(3S)-4-[[4-(6-Amino-3-pyridyl)-1- methyl-pyrrolo[2,3-b]pyridin-2- yl]methyl]-3-methyl-piperazin-1-yl]- N,N-dimethyl-benzamide |
| 1n6 | 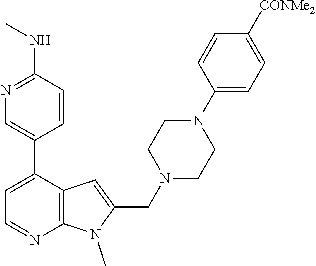 | N,N-Dimethyl-4-[4-[[1-methyl-4-[6- (methylamino)-3-pyridyl]pyrrolo[2,3- b]pyridin-2-yl]methyl]piperazin-1- yl]benzamide |
| 1n7 | 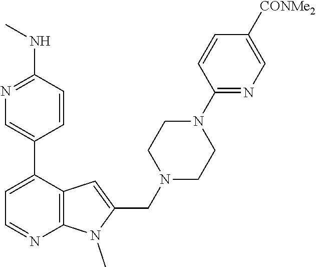 | N,N-Dimethyl-6-[4-[[1-methyl-4-[6- (methylamino)-3-pyridyl]pyrrolo[2,3- b]pyridin-2-yl]methyl]piperazin-1- yl]pyridine-3-carboxamide |
| 1n8 | 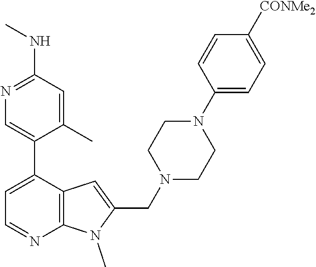 | N,N-Dimethyl-4-[4-[[1-methyl-4-[4- methyl-6-(methylamino)-3- pyridyl]pyrrolo[2,3-b]pyridin-2- yl]methyl]piperazin-1-yl]benzamide |
| 1n9 | 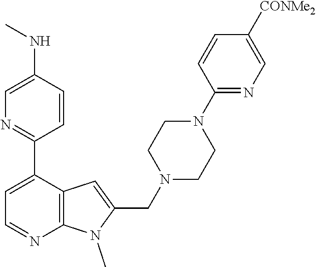 | N,N-Dimethyl-6-[4-[[1-methyl-4-[5- (methylamino)-2-pyridyl]pyrrolo[2,3- b]pyridin-2-yl]methyl]piperazin-1- yl]pyridine-3-carboxamide |
| 1o1 | 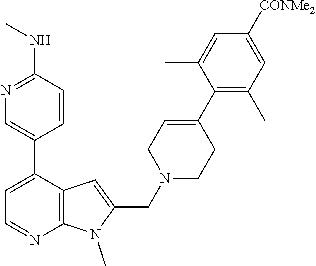 | N,N,3,5-tetramethyl-4-[1-[[1-methyl-4- [5-(methylamino)-2- pyridyl]pyrrolo[2,3-b]pyridin-2- yl]methyl]-3,6-dihydro-2H-pyridin-4- yl]benzamide |
| 1o2 | 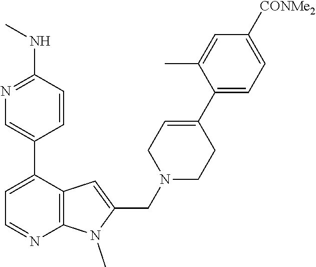 | N,N,3-Trimethyl-4-[1-[[1-methyl-4-[5- (methylamino)-2-pyridyl]pyrrolo[2,3- b]pyridin-2-yl]methyl]-3,6-dihydro- 2H-pyridin-4-yl]benzamide |
| 1o3 | 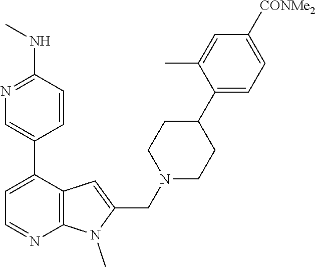 | N,N,3-Trimethyl-4-[1-[[1-methyl-4-[5- (methylamino)-2-pyridyl]pyrrolo[2,3- b]pyridin-2-yl]methyl]-4- piperidyl]benzamide |
| 1o4 | 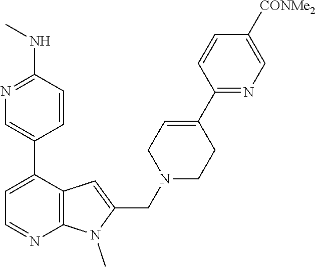 | N,N-Dimethyl-6-[1-[[1-methyl-4-[5- (methylamino)-2-pyridyl]pyrrolo[2,3- b]pyridin-2-yl]methyl]-3,6-dihydro- 2H-pyridin-4-yl]pyridine-3- carboxamide |
| 1o5 | 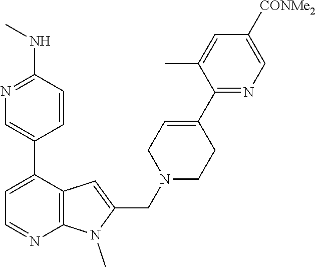 | N,N,5-Trimethyl-6-[1-[[1-methyl-4-[5- (methylamino)-2-pyridyl]pyrrolo[2,3- b]pyridin-2-yl]methyl]-3,6-dihydro- 2H-pyridin-4-yl]pyridine-3- carboxamide |
| 1o6 | 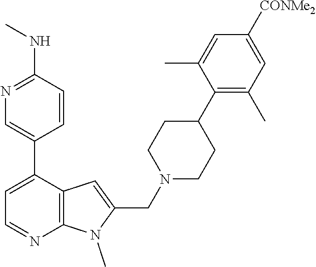 | N,N,3,5-Tetramethyl-4-[1-[[1-methyl- 4-[5-(methylamino)-2- pyridyl]pyrrolo[2,3-b]pyridin-2- yl]methyl]-4-piperidyl]benzamide |
| 1o7 | 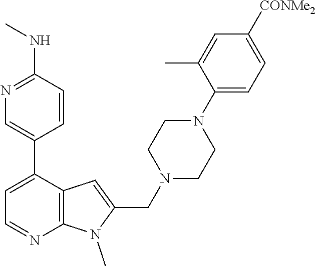 | N,N,3-Trimethyl-4-[4-[[1-methyl-4-[5- (methylamino)-2-pyridyl]pyrrolo[2,3- b]pyridin-2-yl]methyl]piperazin-1- yl]benzamide |
| 1o8 | 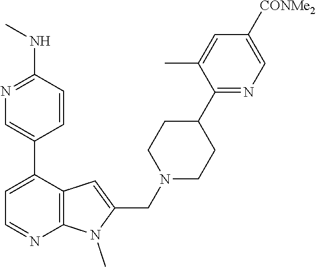 | N,N,5-trimethyl-6-[1-[[1-methyl-4-[5- (methylamino)-2-pyridyl]pyrrolo[2,3- b]pyridin-2-yl]methyl]-4- piperidyl]pyridine-3-carboxamide |
| 1o17 1o18 | 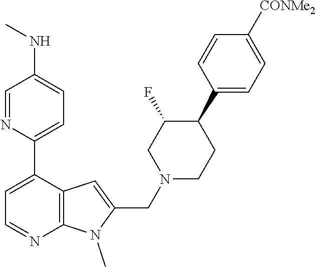 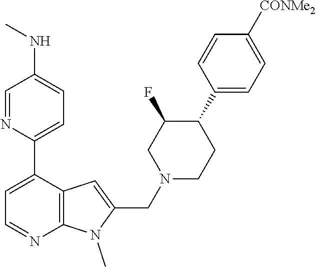 | 4-((3S,4S)-3-fluoro-1-((1-methyl-4-(5- (methylamino)pyridin-2-yl)-1H- pyrrolo[2,3-b]pyridin-2- yl)methyl)piperidin-4-yl)-N,N- dimethylbenzamide and 4-((3R,4R)-3- fluoro-1-((1-methyl-4-(5- (methylamino)pyridin-2-yl)-1H- pyrrolo[2,3-b]pyridin-2- yl)methyl)piperidin-4-yl)-N,N- dimethylbenzamide |
| 1o19 | 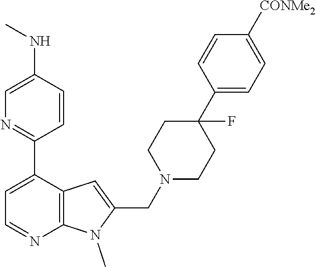 | 4-[4-Fluoro-1-[[1-methyl-4-[5- (methylamino)-2-pyridyl]pyrrolo[2,3- b]pyridin-2-yl]methyl]-4-piperidyl]- N,N-dimethyl-benzamide |
| 1o20 1o21 | 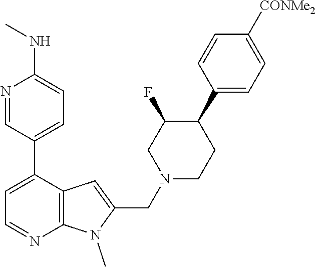 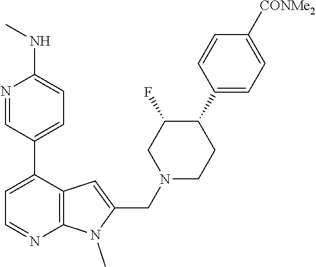 | N,N-Dimethyl-4-[(3S,4R)-3-fluoro-1- [[1-methyl-4-[5-(methylamino)-2- pyridyl]pyrrolo[2,3-b]pyridin-2- yl]methyl]-4-piperidyl]benzamide and N,N-dimethyl-4-[(3R,4S)-3-fluoro-1- [[1-methyl-4-[5-(methylamino)-2- pyridyl]pyrrolo[2,3-b]pyridin-2- yl]methyl]-4-piperidyl]benzamide |
| 1o23 | 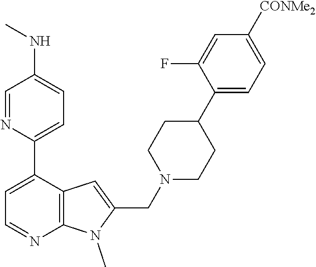 | 3-Fluoro-N,N-dimethyl-4-[1-[[1- methyl-4-[5-(methylamino)-2- pyridyl]pyrrolo[2,3-b]pyridin-2- yl]methyl]-4-piperidyl]benzamide |
| 1o24 1o25 | 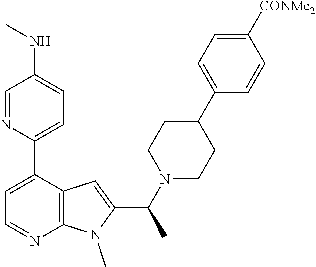 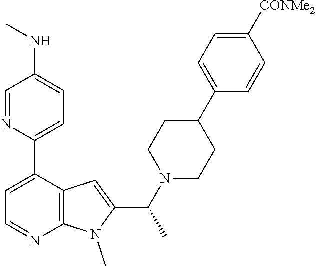 | (S)-N,N-dimethyl-4-(1-(1-(1-methyl-4- (5-(methylamino)pyridin-2-yl)-1H- pyrrolo[2,3-b]pyridin-2- yl)ethyl)piperidin-4-yl)benzamide and (R)-N,N-dimethyl-4-(1-(1-(1-methyl-4- (5-(methyl-amino)pyridin-2-yl)-1H- pyrrolo[2,3-b]pyridin-2- yl)ethyl)piperidin-4-yl)benzamide |
| 1o26 | 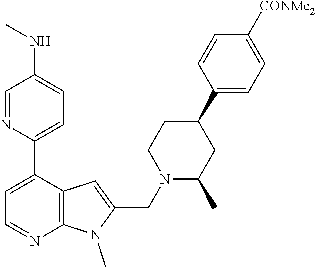 | N,N-dimethyl-4-((2R,4R)-2-methyl-1- ((1-methyl-4-(5-(methylamino)- pyridin-2-yl)-1H-pyrrolo[2,3- b]pyridin-2-yl)methyl)piperidin-4- yl)benzamide |
| 1o27 | 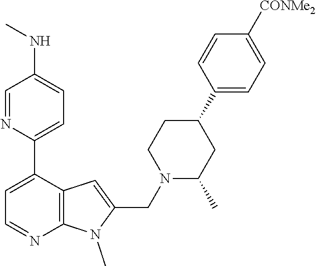 | N,N-dimethyl-4-((2S,4S)-2-methyl-1- ((1-methyl-4-(5-(methylamino)- pyridin-2-yl)-1H-pyrrolo[2,3- b]pyridin-2-yl)methyl)piperidin-4- yl)benzamide |
| 1o28 1o29 | 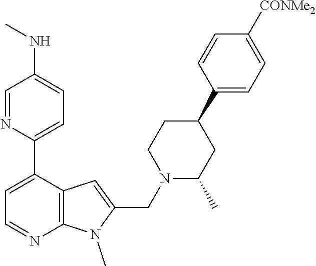 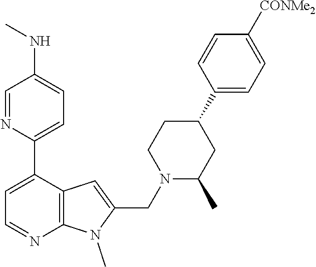 | N,N-dimethyl-4-((2S,4R)-2-methyl-1- ((1-methyl-4-(5- (methylamino)pyridin-2-yl)-1H- pyrrolo[2,3-b]pyridin-2- yl)methyl)piperidin-4-yl)benzamide and N,N-dimethyl-4-((2R,4S)-2- methyl-1-((1-methyl-4-(5- (methylamino)pyridin-2-yl)-1H- pyrrolo[2,3-b]pyridin-2- yl)methyl)piperidin-4-yl)benzamide |
| 1p1 | 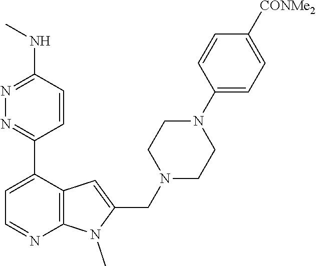 | N,N-Dimethyl-4-[4-[[1-methyl-4-[6- (methylamino)pyridazin-3- yl]pyrrolo[2,3-b]pyridin-2- yl]methyl]piperazin-1-yl]benzamide |
| 1p2 | 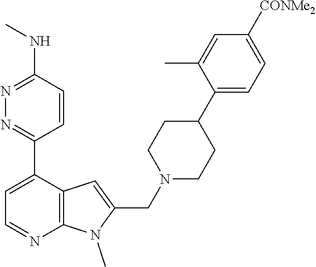 | N,N,3-Trimethyl-4-[1-[[1-methyl-4-[6- (methylamino)pyridazin-3- yl]pyrrolo[2,3-b]pyridin-2-yl]methyl]- 4-piperidyl]benzamide |
| 1p3 | 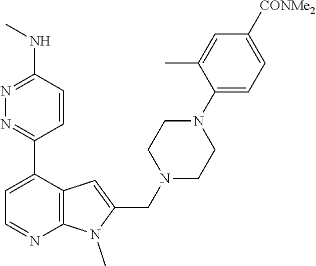 | N,N,3-Trimethyl-4-[4-[[1-methyl-4-[6- (methylamino)pyridazin-3- yl]pyrrolo[2,3-b]pyridin-2- yl]methyl]piperazin-1-yl]benzamide |
| 1p4 | 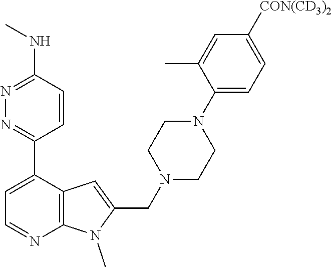 | 3-Methyl-4-[4-[[1-methyl-4-[6- (methylamino)pyridazin-3- yl]pyrrolo[2,3-b]pyridin-2- yl]methyl]piperazin-1-yl]-N,N- bis(trideuteriomethyl)benzamide |
| 1p5 | 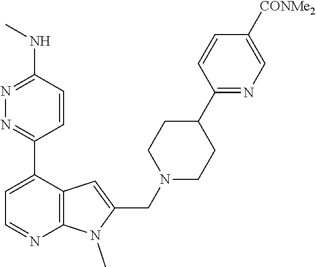 | N,N-Dimethyl-6-[1-[[1-methyl-4-[6- (methylamino)pyridazin-3- yl]pyrrolo[2,3-b]pyridin-2-yl]methyl]- 4-piperidyl]pyridine-3-carboxamide |
| 1p6 | 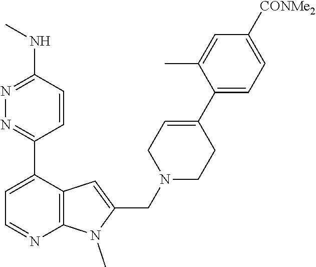 | N,N,3-Trimethyl-4-[1-[[1-methyl-4-[6- (methylamino)pyridazin-3- yl]pyrrolo[2,3-b]pyridin-2-yl]methyl]- 3,6-dihydro-2H-pyridin-4- yl]benzamide |
| 1p7 | 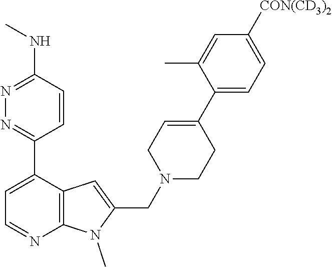 | 3-Methyl-4-[1-[[1-methyl-4-[6- (methylamino)pyridazin-3- yl]pyrrolo[2,3-b]pyridin-2-yl]methyl]- 3,6-dihydro-2H-pyridin-4-yl]-N,N- bis(trideuteriomethyl)benzamide |
| 1p8 | 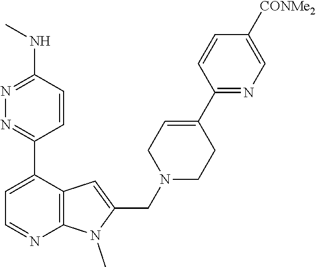 | N,N-Dimethyl-6-[1-[[1-methyl-4-[6- (methylamino)pyridazin-3- yl]pyrrolo[2,3-b]pyridin-2-yl]methyl]- 3,6-dihydro-2H-pyridin-4-yl]pyridine- 3-carboxamide |
| 1p9 | 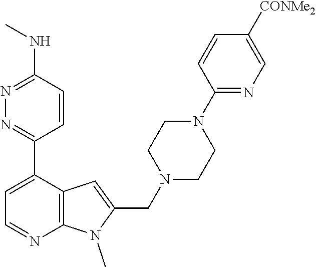 | N,N-Dimethyl-6-[4-[[1-methyl-4-[6- (methylamino)pyridazin-3- yl]pyrrolo[2,3-b]pyridin-2- yl]methyl]piperazin-1-yl]pyridine-3- carboxamide |
| 1p10 | 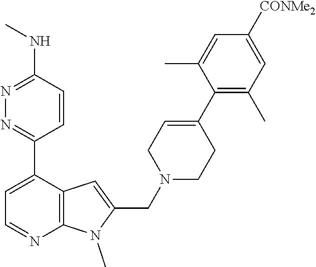 | N,N,3,5-Tetramethyl-4-[1-[[1-methyl- 4-[6-(methylamino)pyridazin-3- yl]pyrrolo[2,3-b]pyridin-2-yl]methyl]- 3,6-dihydro-2H-pyridin-4- yl]benzamide |
| 1p11 | 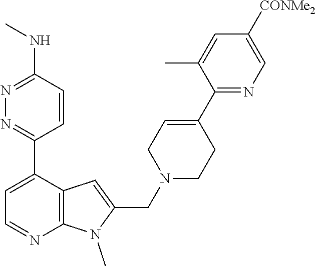 | N,N,5-Trimethyl-6-[1-[[1-methyl-4-[6- (methylamino)pyridazin-3- yl]pyrrolo[2,3-b]pyridin-2-yl]methyl]- 3,6-dihydro-2H-pyridin-4-yl]pyridine- 3-carboxamide |
| 1p14 | 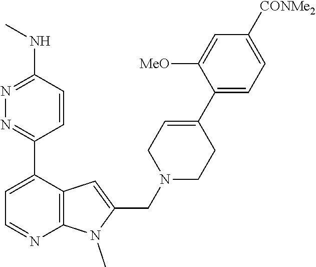 | 3-Methoxy-N,N-dimethyl-4-[1-[[1- methyl-4-[6-(methylamino)pyridazin- 3-yl]pyrrolo[2,3-b]pyridin-2- yl]methyl]-3,6-dihydro-2H-pyridin-4- yl]benzamide |
| 1p15 | 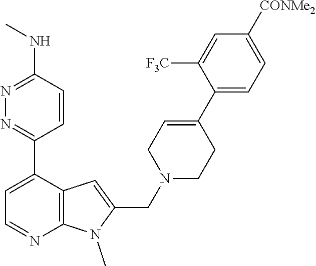 | N,N-Dimethyl-4-[1-[[1-methyl-4-[6- (methylamino)pyridazin-3- yl]pyrrolo[2,3-b]pyridin-2-yl]methyl]- 3,6-dihydro-2H-pyridin-4-yl]-3- (trifluoromethyl)benzamide |
| 1p17 | 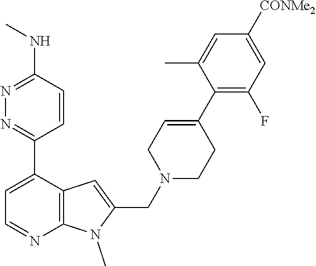 | 3-Fluoro-N,N,5-trimethyl-4-[1-[[1- methyl-4-[6-(methylamino)pyridazin- 3-yl]pyrrolo[2,3-b]pyridin-2- yl]methyl]-3,6-dihydro-2H-pyridin-4- yl]benzamide |
| 1p18 | 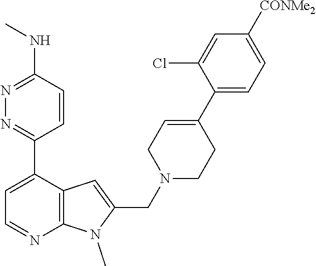 | 3-Chloro-N,N-dimethyl-4-[1-[[1- methyl-4-[6-(methylamino)pyridazin- 3-yl]pyrrolo[2,3-b]pyridin-2- yl]methyl]-3,6-dihydro-2H-pyridin-4- yl]benzamide |
| 1p20 | 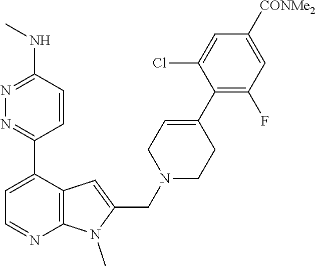 | 3-Chloro-5-fluoro-N,N-dimethyl-4-[1- [[1-methyl-4-[6- (methylamino)pyridazin-3- yl]pyrrolo[2,3-b]pyridin-2-yl]methyl]- 3,6-dihydro-2H-pyridin-4- yl]benzamide |
| 1q1 1q6 | 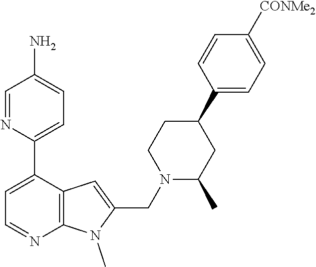 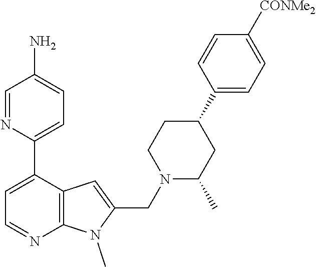 | N,N-Dimethyl-4-[(2R,4R)-1-[[4-(5- amino-2-pyridyl)-1-methyl- pyrrolo[2,3-b]pyridin-2-yl]methyl]-2- methyl-4-piperidyl]benzamide and N,N-dimethyl-4-[(2S,4S)-1-[[4-(5- amino-2-pyridyl)-1-methyl- pyrrolo[2,3-b]pyridin-2-yl]methyl]-2- methyl-4-piperidyl]benzamide |
| 1q2 1q3 | 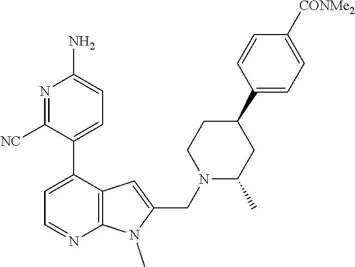 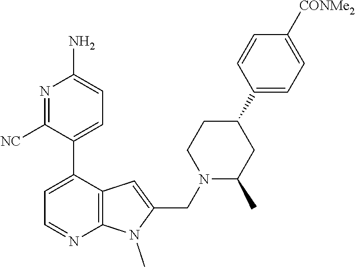 | N,N-Dimethyl-4-[(2S,4R)-1-[[4-(6- amino-2-cyano-3-pyridyl)-1-methyl- pyrrolo[2,3-b]pyridin-2-yl]methyl]-2- methyl-4-piperidyl]benzamide and N,N-dimethyl-4-[(2R,4S)-1-[[4-(6- amino-2-cyano-3-pyridyl)-1-methyl- pyrrolo[2,3-b]pyridin-2-yl]methyl]-2- methyl-4-piperidyl]benzamide |
| 1q4 1q5 | 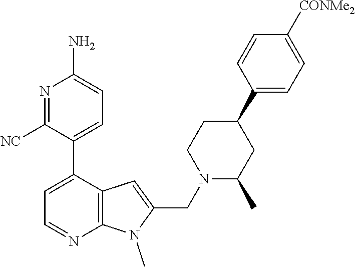 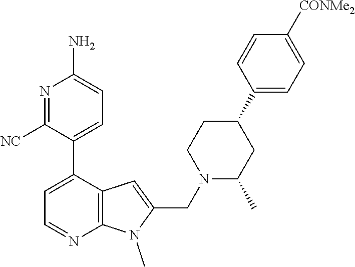 | N,N-Dimethyl-4-[(2R,4R)-1-[[4-(6- amino-2-cyano-3-pyridyl)-1-methyl- pyrrolo[2,3-b]pyridin-2-yl]methyl]-2- methyl-4-piperidyl]benzamide and N,N-dimethyl-4-[(2S,4S)-1-[[4-(6- amino-2-cyano-3-pyridyl)-1-methyl- pyrrolo[2,3-b]pyridin-2-yl]methyl]-2- methyl-4-piperidyl]benzamide |
| 1q7 1q8 | 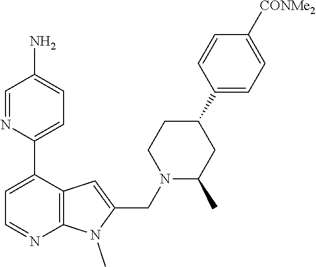 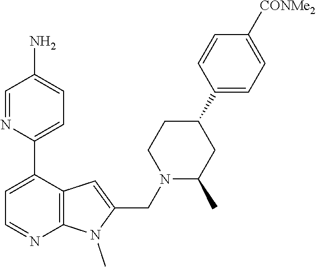 | N,N-Dimethyl-4-[(2R,4S)-1-[[4-(5- amino-2-pyridyl)-1-methyl- pyrrolo[2,3-b]pyridin-2-yl]methyl]-2- methyl-4-piperidyl]benzamide and N,N-dimethyl-4-[(2S,4R)-1-[[4-(5- amino-2-pyridyl)-1-methyl- pyrrolo[2,3-b]pyridin-2-yl]methyl]-2- methyl-4-piperidyl]benzamide |
| 1r5 1r6 | 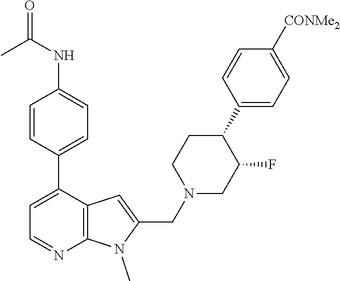 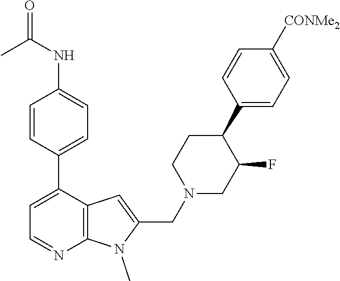 | N,N-Dimethyl-4-[(3S,4R)-1-[[4-(4- acetamidophenyl)-1-methyl- pyrrolo[2,3-b]pyridin-2-yl]methyl]-3- fluoro-4-piperidyl]benzamide and N,N- dimethyl-4-[(3R,4S)-1-[[4-(4- acetamidophenyl)-1-methyl- pyrrolo[2,3-b]pyridin-2-yl]methyl]-3- fluoro-4-piperidyl]benzamide |
| 1s1 | 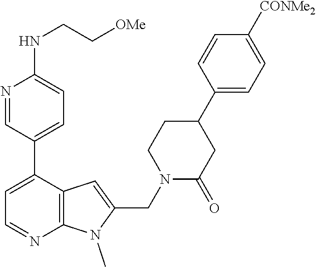 | 4-[1-[[4-[6-(2-Methoxyethylamino)-3- pyridyl]-1-methyl-pyrrolo[2,3- b]pyridin-2-yl]methyl]-2-oxo-4- piperidyl]-N,N-dimethyl-benzamide |
| 1s2 | 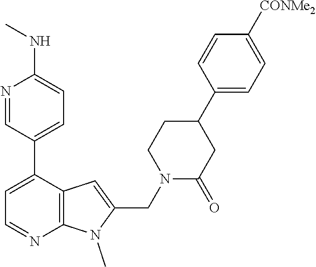 | N,N-Dimethyl-4-[1-[[1-methyl-4-[6- (methylamino)-3-pyridyl]pyrrolo[2,3- b]pyridin-2-yl]methyl]-2-oxo-4- piperidyl]benzamide |
| 1s3 1s4 | 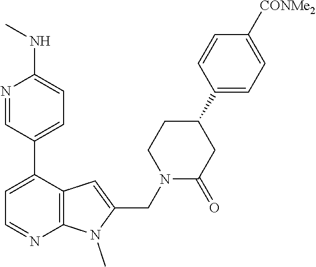 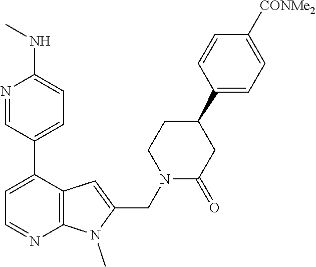 | N,N-Dimethyl-4-[(4R)-1-[[1-methyl-4- [6-(methylamino)-3- pyridyl]pyrrolo[2,3-b]pyridin-2- yl]methyl]-2-oxo-4- piperidyl]benzamide and N,N- dimethyl-4-[(4S)-1-[[1-methyl-4-[6- (methylamino)-3-pyridyl]pyrrolo[2,3- b]pyridin-2-yl]methyl]-2-oxo-4- piperidyl]benzamide |
| 1s5 | 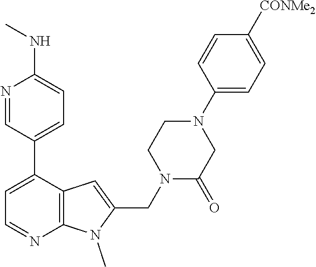 | N,N-Dimethyl-4-[4-[[1-methyl-4-[6- (methylamino)-3-pyridyl]pyrrolo[2,3- b]pyridin-2-yl]methyl]-3-oxo- piperazin-1-yl]benzamide |
| 1s6 | 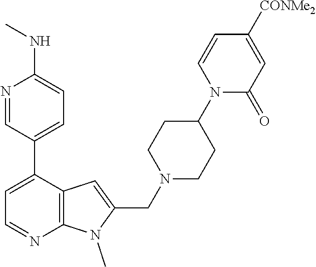 | N,N-Dimethyl-1-[1-[[1-methyl-4-[6- (methylamino)-3-pyridyl]pyrrolo[2,3- b]pyridin-2-yl]methyl]-4-piperidyl]-2- oxo-pyridine-4-carboxamide |
| 1s8 | 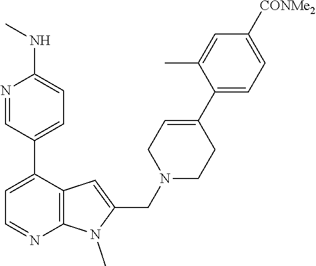 | N,N,3-Trimethyl-4-[1-[[1-methyl-4-[6- (methylamino)-3-pyridyl]pyrrolo[2,3- b]pyridin-2-yl]methyl]-3,6-dihydro- 2H-pyridin-4-yl]benzamide |
| 1s9 | 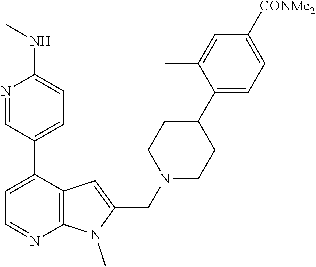 | N,N,3-Trimethyl-4-[1-[[1-methyl-4-[6- (methylamino)-3-pyridyl]pyrrolo[2,3- b]pyridin-2-yl]methyl]-4- piperidyl]benzamide |
| 1s10 | 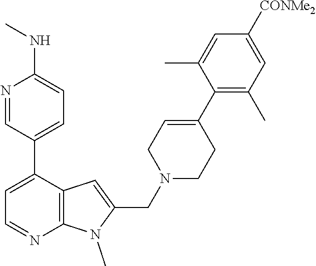 | N,N,3,5-Tetramethyl-4-[1-[[1-methyl- 4-[6-(methylamino)-3- pyridyl]pyrrolo[2,3-b]pyridin-2- yl]methyl]-3,6-dihydro-2H-pyridin-4- yl]benzamide |
| 1s11 | 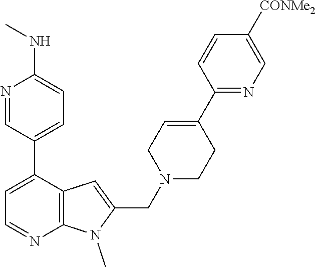 | N,N-Dimethyl-6-[1-[[1-methyl-4-[6- (methylamino)-3-pyridyl]pyrrolo[2,3- b]pyridin-2-yl]methyl]-3,6-dihydro- 2H-pyridin-4-yl]pyridine-3- carboxamide |
| 1s12 | 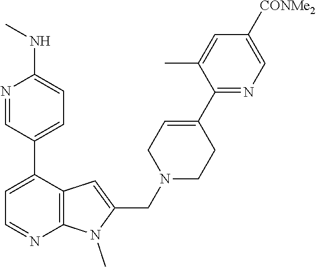 | N,N,5-Trimethyl-6-[1-[[1-methyl-4-[6- (methylamino)-3-pyridyl]pyrrolo[2,3- b]pyridin-2-yl]methyl]-3,6-dihydro- 2H-pyridin-4-yl]pyridine-3- carboxamide |
| 1s13 | 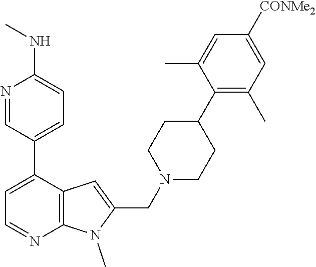 | N,N,3,5-Tetramethyl-4-[1-[[1-methyl- 4-[6-(methylamino)-3- pyridyl]pyrrolo[2,3-b]pyridin-2- yl]methyl]-4-piperidyl]benzamide |
| 1s14 | 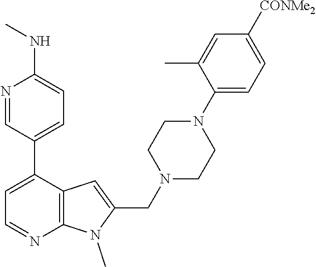 | N,N,3-Trimethyl-4-[4-[[1-methyl-4-[6- (methylamino)-3-pyridyl]pyrrolo[2,3- b]pyridin-2-yl]methyl]piperazin-1- yl]benzamide |
| 1s15 | 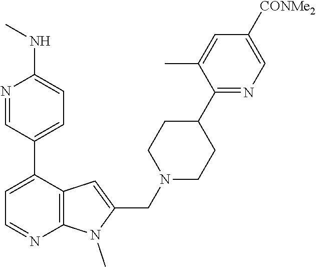 | N,N,5-Trimethyl-6-[1-[[1-methyl-4-[6- (methylamino)-3-pyridyl]pyrrolo[2,3- b]pyridin-2-yl]methyl]-4- piperidyl]pyridine-3-carboxamide |
| 1s24 1s25 | 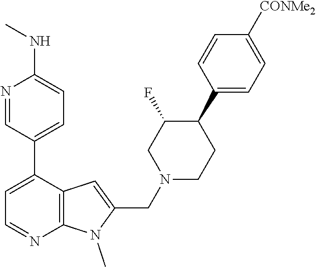 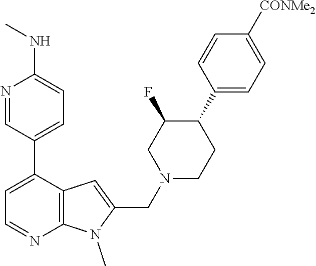 | N,N-Dimethyl-4-[(3R,4R)-3-fluoro-1- [[1-methyl-4-[6-(methylamino)-3- pyridyl]pyrrolo[2,3-b]pyridin-2- yl]methyl]-4-piperidyl]benzamide and N,N-dimethyl-4-[(3S,4S)-3-fluoro-1- [[1-methyl-4-[6-(methylamino)-3- pyridyl]pyrrolo[2,3-b]pyridin-2- yl]methyl]-4-piperidyl]benzamide |
| 1s26 | 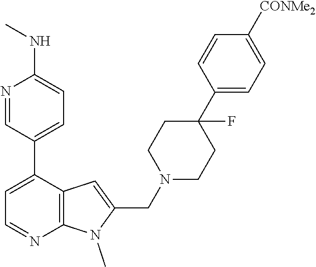 | 4-[4-Fluoro-1-[[1-methyl-4-[6- (methylamino)-3-pyridyl]pyrrolo[2,3- b]pyridin-2-yl]methyl]-4-piperidyl]- N,N-dimethyl-benzamide |
| 1s27 1s28 | 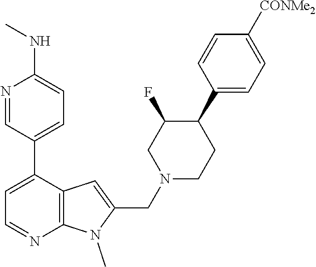 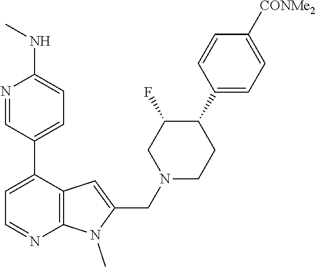 | N,N-Dimethyl-4-[(3R,4S)-3-fluoro-1- [[1-methyl-4-[6-(methylamino)-3- pyridyl]pyrrolo[2,3-b]pyridin-2- yl]methyl]-4-piperidyl]benzamide and N,N-dimethyl-4-[(3S,4R)-3-fluoro-1- [[1-methyl-4-[6-(methylamino)-3- pyridyl]pyrrolo[2,3-b]pyridin-2- yl]methyl]-4-piperidyl]benzamide |
| 1s30 | 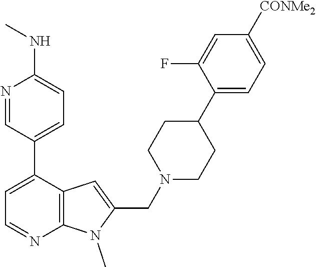 | 3-Fluoro-N,N-dimethyl-4-(1-((1- methyl-4-(6-(methylamino)pyridin-3- yl)-1H-pyrrolo[2,3-b]pyridin-2- yl)methyl)piperidin-4-yl)benzamide |
| 1u2 | 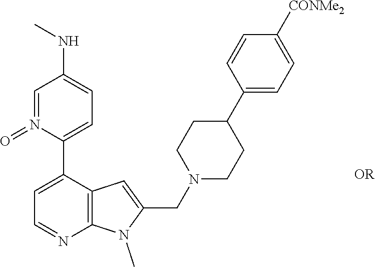 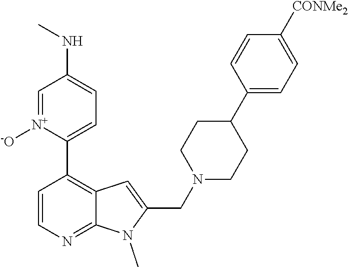 | N,N-Dimethyl-4-[1-[[1-methyl-4-[5- (methylamino)-1-oxido-pyridin-1-ium- 2-yl]pyrrolo[2,3-b]pyridin-2- yl]methyl]-4-piperidyl]benzamide |
| 1u3 | 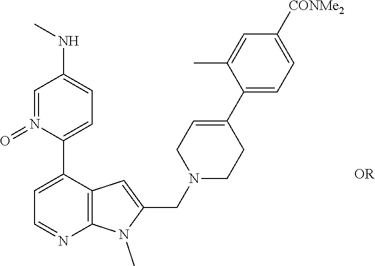 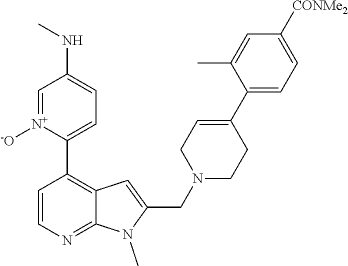 | N,N,3-Trimethyl-4-[1-[[1-methyl-4-[5- (methylamino)-1-oxido-pyridin-1-ium- 2-yl]pyrrolo[2,3-b]pyridin-2- yl]methyl]-3,6-dihydro-2H-pyridin-4- yl]benzamide |
| 1u5 | 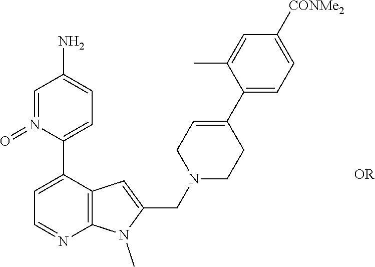 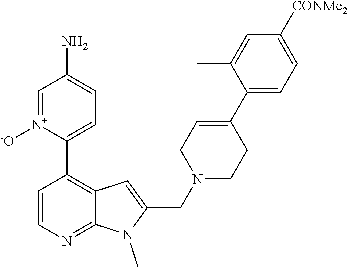 | 4-[1-[[4-(5-Amino-1-oxido-pyridin-1- ium-2-yl)-1-methyl-pyrrolo[2,3- b]pyridin-2-yl]methyl]-4-piperidyl]- N,N-dimethyl-benzamide |
| 1x1 | 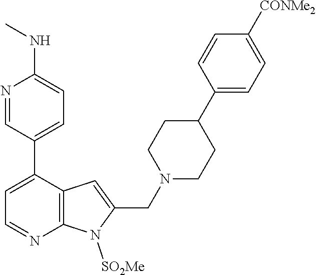 | N,N-Dimethyl-4-[1-[[4-[6- (methylamino)-3-pyridyl]-1- methylsulfonyl-pyrrolo[2,3-b]pyridin- 2-yl]methyl]-4-piperidyl]benzamide |
| 1x2 | 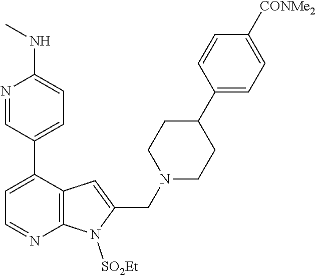 | 4-[1-[[1-Ethylsulfonyl-4-[6- (methylamino)-3-pyridyl]pyrrolo[2,3- b]pyridin-2-yl]methyl]-4-piperidyl]- N,N-dimethyl-benzamide |
| 1x3 | 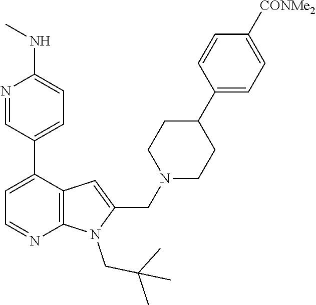 | 4-[1-[[1-(2,2-Dimethylpropyl)-4-[6- (methylamino)-3-pyridyl]pyrrolo[2,3- b]pyridin-2-yl]methyl]-4-piperidyl]- N,N-dimethyl-benzamide |
| 1yl | 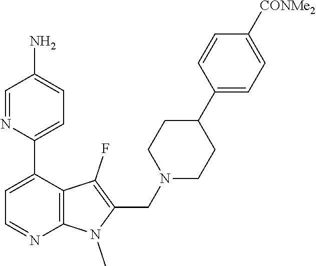 | 4-(1-((4-(5-Aminopyridin-2-yl)-3- fluoro-1-methyl-1H-pyrrolo[2,3- b]pyridin-2-yl)methyl)piperidin-4-yl)- N,N-dimethylbenzamide |
| 1z1 | 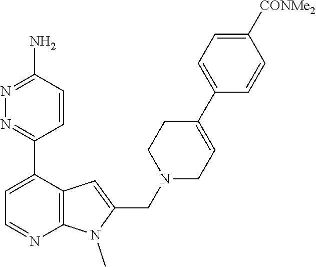 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-N,N-dimethylbenzamide |
| 1z2 | 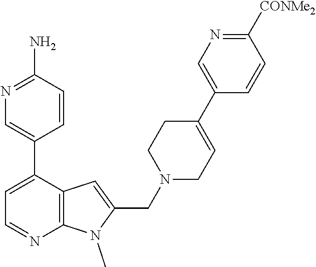 | 1′-((4-(6-aminopyridin-3-yl)-1-methyl- 1H-pyrrolo[2,3-b]pyridin-2- yl)methyl)-N,N-dimethyl-1′,2′,3′,6′- tetrahydro-[3,4′-bipyridine]-6- carboxamide |
| 1z3 | 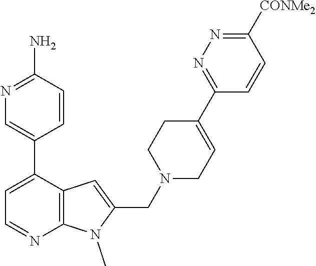 | 6-(1-((4-(6-aminopyridin-3-yl)-1- methyl-1H-pyrrolo[2,3-b]pyridin-2- yl)methyl)-1,2,3,6-tetrahydropyridin- 4-yl)-N,N-dimethylpyridazine-3- carboxamide |
| 1z4 | 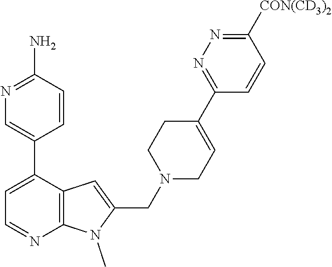 | 6-(1-((4-(6-aminopyridin-3-yl)-1- methyl-1H-pyrrolo[2,3-b]pyridin-2- yl)methyl)-1,2,3,6-tetrahydropyridin- 4-yl)-N,N-bis(methyl-d₃)pyridazine-3- carboxamide |
| 1z5 | 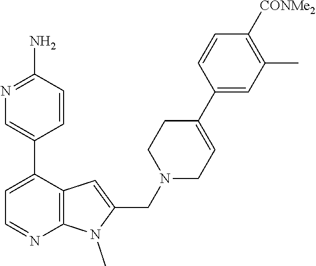 | 4-(1-((4-(6-aminopyridin-3-yl)-1- methyl-1H-pyrrolo[2,3-b]pyridin-2- yl)methyl)-1,2,3,6-tetrahydropyridin- 4-yl)-N,N,2-trimethylbenzamide |
| 1z6 | 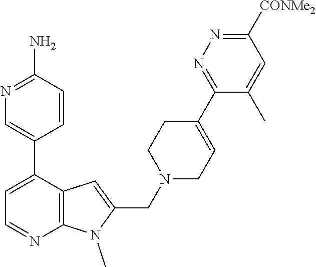 | 6-(1-((4-(6-aminopyridin-3-yl)-1- methyl-1H-pyrrolo[2,3-b]pyridin-2- yl)methyl)-1,2,3,6-tetrahydropyridin- 4-yl)-N,N,5-trimethylpyridazine-3- carboxamide |
| 1z7 | 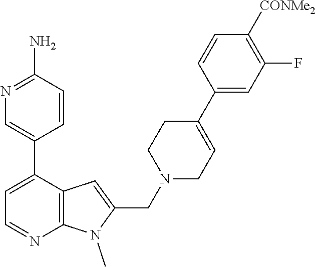 | 4-(1-((4-(6-aminopyridin-3-yl)-1- methyl-1H-pyrrolo[2,3-b]pyridin-2- yl)methyl)-1,2,3,6-tetrahydropyridin- 4-yl)-2-fluoro-N,N- dimethylbenzamide |
| 1z9 | 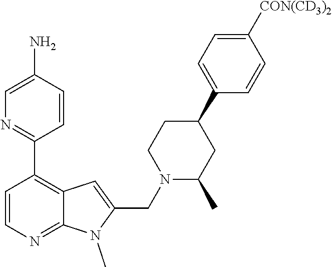 | 4-((2R,4R)-1-((4-(5-aminopyridin-2- yl)-1-methyl-1H-pyrrolo[2,3- b]pyridin-2-yl)methyl)-2- methylpiperidin-4-yl)-N,N-bis(methyl- d₃)benzamide |
| 1z10 | 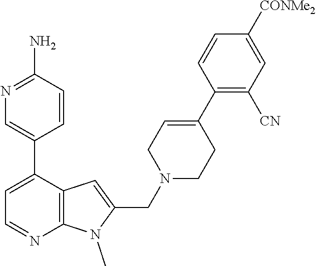 | 4-(1-((4-(6-aminopyridin-3-yl)-1- methyl-1H-pyrrolo[2,3-b]pyridin-2- yl)methyl)-1,2,3,6-tetrahydropyridin- 4-yl)-3-cyano-N,N-dimethylbenzamide |
| 1z11 | 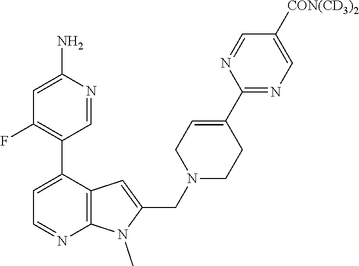 | 2-(1-((4-(6-amino-4-fluoropyridin-3- yl)-1-methyl-1H-pyrrolo[2,3- b]pyridin-2-yl)methyl)-1,2,3,6- tetrahydropyridin-4-yl)-N,N- bis(methyl-d₃)pyrimidine-5- carboxamide |
| 1z12 | 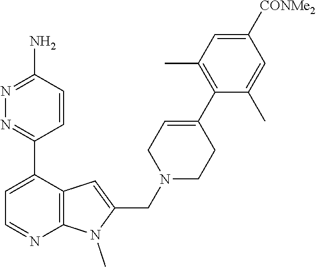 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-N,N,3,5-tetramethylbenzamide |
| 1z13 | 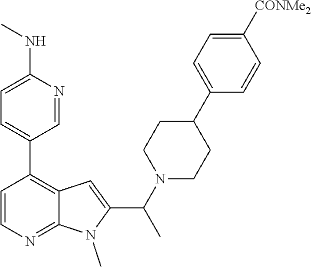 | N,N-dimethyl-4-(1-(1-(1-methyl-4-(6- (methylamino)pyridin-3-yl)-1H- pyrrolo[2,3-b]pyridin-2- yl)ethyl)piperidin-4-yl)benzamide |
| 1z14 | 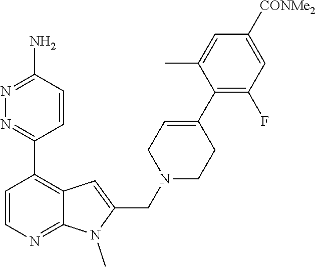 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-3-fluoro-N,N,5- trimethylbenzamide |
| 1z15 | 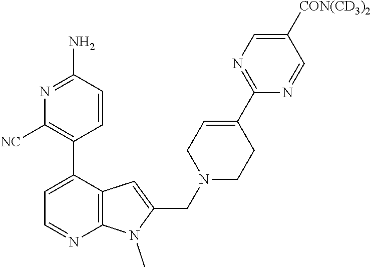 | 2-(1-((4-(6-amino-2-cyanopyridin-3- yl)-1-methyl-1H-pyrrolo[2,3- b]pyridin-2-yl)methyl)-1,2,3,6- tetrahydropyridin-4-yl)-N,N- bis(methyl-d₃)pyrimidine-5- carboxamide |
| 1z16 | 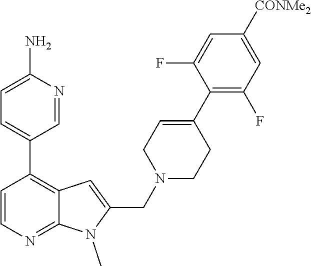 | 4-(1-((4-(6-aminopyridin-3-yl)-1- methyl-1H-pyrrolo[2,3-b]pyridin-2- yl)methyl)-1,2,3,6-tetrahydropyridin- 4-yl)-3,5-difluoro-N,N- dimethylbenzamide |
| 1z17 | 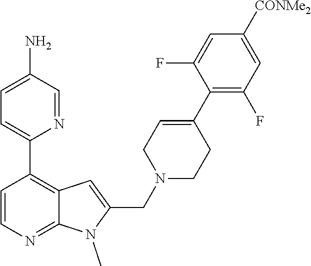 | 4-(1-((4-(5-aminopyridin-2-yl)-1- methyl-1H-pyrrolo[2,3-b]pyridin-2- yl)methyl)-1,2,3,6-tetrahydropyridin- 4-yl)-3,5-difluoro-N,N- dimethylbenzamide |
| 1z18 | 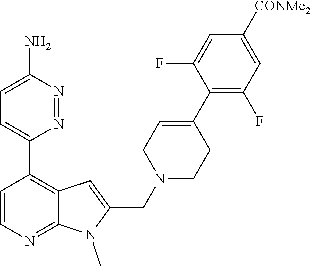 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-3,5-difluoro-N,N- dimethylbenzamide |
| 1z19 | 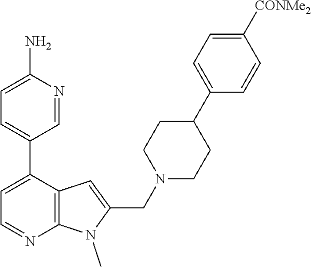 | 4-(1-((4-(6-aminopyridin-3-yl)-1- methyl-1H-pyrrolo[2,3-b]pyridin-2- yl)methyl)piperidin-4-yl)-N,N- dimethylbenzamide |
| 1z20 | 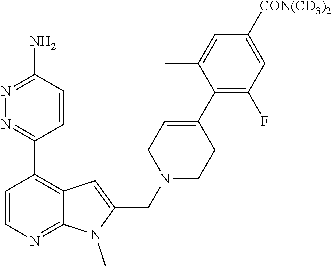 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-3-fluoro-5-methyl-N,N- bis(methyl-d₃)benzamide |
| 1z21 | 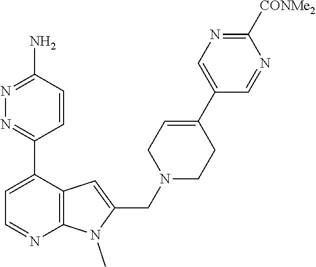 | 5-(1-((4-(6-aminopyridazin-3-yl)-1- methyl-1H-pyrrolo[2,3-b]pyridin-2- yl)methyl)-1,2,3,6-tetrahydropyridin- 4-yl)-N,N-dimethylpyrimidine-2- carboxamide |
| 1z22 | 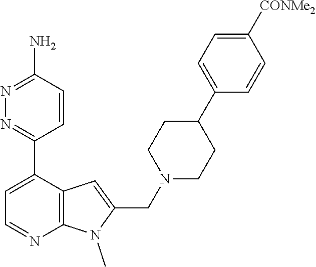 | 4-(1-((4-(6-aminopyridazin-3-yl)-1- methyl-1H-pyrrolo[2,3-b]pyridin-2- yl)methyl)piperidin-4-yl)-N,N- dimethylbenzamide |
| 1z23 | 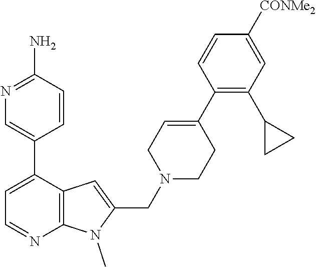 | 4-(1-((4-(6-aminopyridin-3-yl)-1- methyl-1H-pyrrolo[2,3-b]pyridin-2- yl)methyl)-1,2,3,6-tetrahydropyridin- 4-yl)-3-cyclopropyl-N,N- dimethylbenzamide |
| 1z24 | 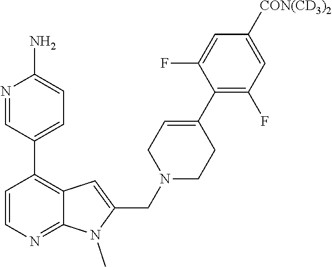 | 4-(1-((4-(6-aminopyridin-3-yl)-1- methyl-1H-pyrrolo[2,3-b]pyridin-2- yl)methyl)-1,2,3,6-tetrahydropyridin- 4-yl)-3,5-difluoro-N,N-bis(methyl- d₃)benzamide |
| 1z25 | 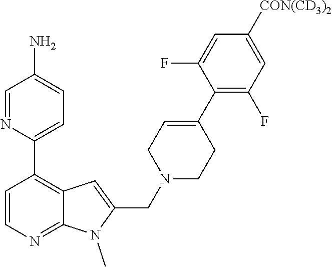 | 4-(1-((4-(5-aminopyridin-2-yl)-1- methyl-1H-pyrrolo[2,3-b]pyridin-2- yl)methyl)-1,2,3,6-tetrahydropyridin- 4-yl)-3,5-difluoro-N,N-bis(methyl- d₃)benzamide |
| 1z27 | 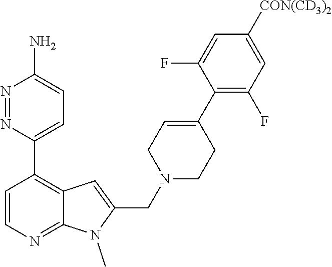 | 4-(1-((4-(6-aminopyridazin-3-yl)-1- methyl-1H-pyrrolo[2,3-b]pyridin-2- yl)methyl)-1,2,3,6-tetrahydropyridin- 4-yl)-3,5-difluoro-N,N-bis(methyl- d₃)benzamide |
| 1z28 1z29 1z30 | 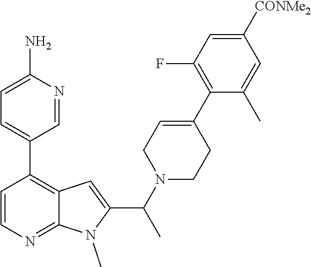 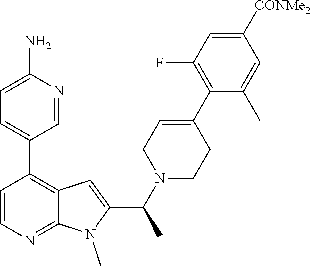 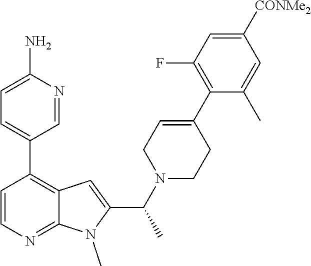 | 4-(1-(1-(4-(6-aminopyridin-3-yl)-1- methyl-1H-pyrrolo[2,3-b]pyridin-2- yl)ethyl)-1,2,3,6-tetrahydropyridin-4- yl)-3-fluoro-N,N,5- trimethylbenzamide, (S)-4-(1-(1-(4-(6- aminopyridin-3-yl)-1-methyl-1H- pyrrolo[2,3-b]pyridin-2-yl)ethyl)- 1,2,3,6-tetrahydropyridin-4-yl)-3- fluoro-N,N,5-trimethylbenzamide and (R)-4-(1-(1-(4-(6-aminopyridin-3-yl)- 1-methyl-1H-pyrrolo[2,3-b]pyridin-2- yl)ethyl)-1,2,3,6-tetrahydropyridin-4- yl)-3-fluoro-N,N,5- trimethylbenzamide |
| 1z31 1z32 1z33 | 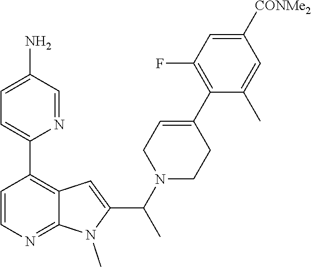 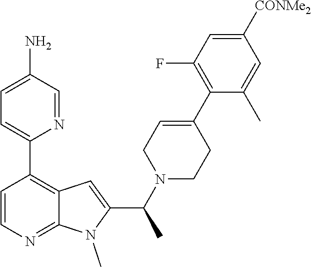 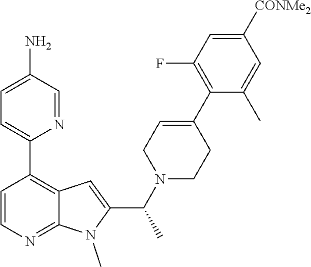 | 4-(1-(1-(4-(5-aminopyridin-2-yl)-1- methyl-1H-pyrrolo[2,3-b]pyridin-2- yl)ethyl)-1,2,3,6-tetrahydropyridin-4- yl)-3-fluoro-N,N,5- trimethylbenzamide, (S)-4-(1-(1-(4-(5- aminopyridin-2-yl)-1-methyl-1H- pyrrolo[2,3-b]pyridin-2-yl)ethyl)- 1,2,3,6-tetrahydropyridin-4-yl)-3- fluoro-N,N,5-trimethylbenzamide and (R)-4-(1-(1-(4-(5-aminopyridin-2-yl)- 1-methyl-1H-pyrrolo[2,3-b]pyridin-2- yl)ethyl)-1,2,3,6-tetrahydropyridin-4- yl)-3-fluoro-N,N,5- trimethylbenzamide |
| 1z34 | 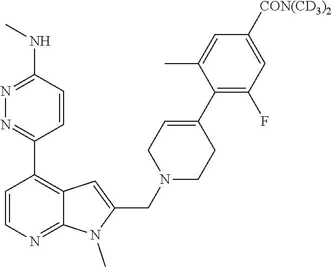 | 3-fluoro-5-methyl-N,N-bis(methyl-d₃)- 4-(1-((1-methyl-4-(6- (methylamino)pyridazin-3-yl)-1H- pyrrolo[2,3-b]pyridin-2-yl)methyl)- 1,2,3,6-tetrahydropyridin-4- yl)benzamide |
| 1z35 | 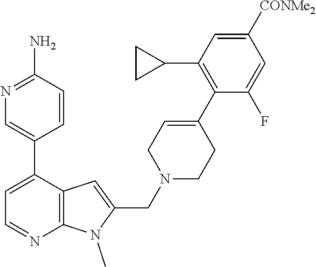 | 4-(1-((4-(6-aminopyridin-3-yl)-1- methyl-1H-pyrrolo[2,3-b]pyridin-2- yl)methyl)-1,2,3,6-tetrahydropyridin- 4-yl)-3-cyclopropyl-5-fluoro-N,N- dimethylbenzamide |
| 1z36 1z37 1z38 | 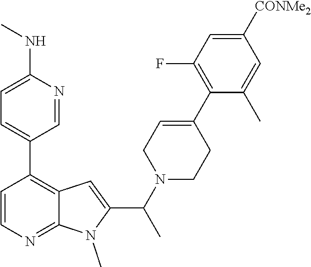 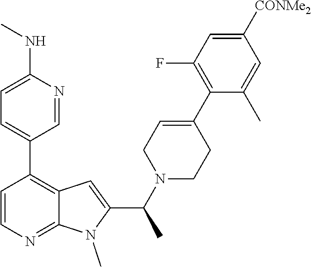 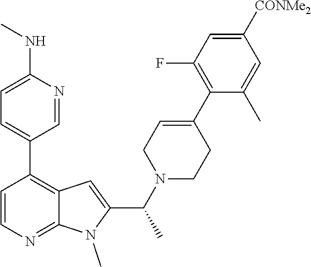 | 3-fluoro-N,N,5-trimethyl-4-(1-(1-(1- methyl-4-(6-(methylamino)pyridin-3- yl)-1H-pyrrolo[2,3-b]pyridin-2- yl)ethyl)-1,2,3,6-tetrahydropyridin-4- yl)benzamide, (S)-3-fluoro-N,N,5- trimethyl-4-(1-(1-(1-methyl-4-(6- (methylamino)pyridin-3-yl)-1H- pyrrolo[2,3-b]pyridin-2-yl)ethyl)- 1,2,3,6-tetrahydropyridin-4- yl)benzamide and (R)-3-fluoro-N,N,5- trimethyl-4-(1-(1-(1-methyl-4-(6- (methyl-amino)pyridin-3-yl)-1H- pyrrolo[2,3-b]pyridin-2-yl)ethyl)- 1,2,3,6-tetrahydropyridin-4- yl)benzamide |
| 1z39 1z40 1z41 | 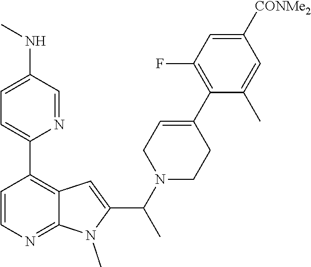 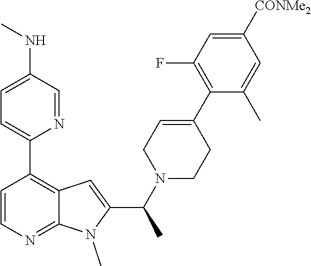 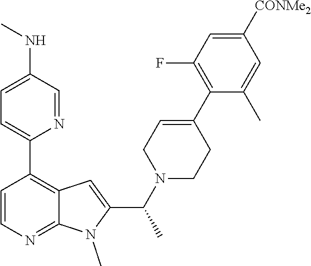 | 3-fluoro-N,N,5-trimethyl-4-(1-(1-(1- methyl-4-(5-(methylamino)pyridin-2- yl)-1H-pyrrolo[2,3-b]pyridin-2- yl)ethyl)-1,2,3,6-tetrahydropyridin-4- yl)benzamide, (S)-3-fluoro-N,N,5- trimethyl-4-(1-(1-(1-methyl-4-(5- (methylamino)pyridin-2-yl)-1H- pyrrolo[2,3-b]pyridin-2-yl)ethyl)- 1,2,3,6-tetrahydropyridin-4- yl)benzamide and (R)-3-fluoro-N,N,5- trimethyl-4-(1-(1-(1-methyl-4-(5- (methyl-amino)pyridin-2-yl)-1H- pyrrolo[2,3-b]pyridin-2-yl)ethyl)- 1,2,3,6-tetrahydropyridin-4- yl)benzamide |
| 1z42 | 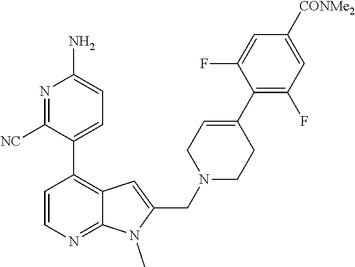 | 4-(1-((4-(6-amino-2-cyanopyridin-3- yl)-1-methyl-1H-pyrrolo[2,3- b]pyridin-2-yl)methyl)-1,2,3,6- tetrahydropyridin-4-yl)-3,5-difluoro- N,N-dimethylbenzamide |
| 1z43 | 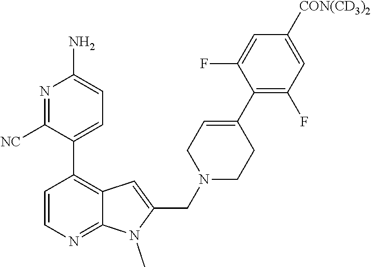 | 4-(1-((4-(6-amino-2-cyanopyridin-3- yl)-1-methyl-1H-pyrrolo[2,3- b]pyridin-2-yl)methyl)-1,2,3,6- tetrahydropyridin-4-yl)-3,5-difluoro- N,N-bis(methyl-d₃)benzamide |
| 1z44 | 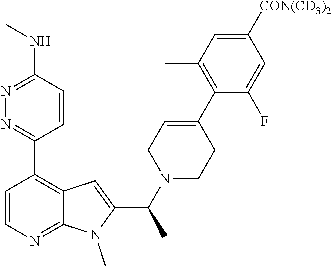 | (S)-3-fluoro-5-methyl-N,N-bis(methyl- d₃)-4-(1-(1-(1-methyl-4-(6- (methylamino)-pyridazin-3-yl)-1H- pyrrolo[2,3-b]pyridin-2-yl)ethyl)- 1,2,3,6-tetrahydropyridin-4- yl)benzamide |
| 1z45 | 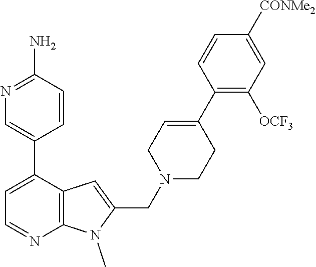 | 4-(1-((4-(6-aminopyridin-3-yl)-1- methyl-1H-pyrrolo[2,3-b]pyridin-2- yl)methyl)-1,2,3,6-tetrahydropyridin- 4-yl)-N,N-dimethyl-3- (trifluoromethoxy)benzamide |

In one or more embodiments of the present disclosure, the compounds of general formula (I) have an (EC₅₀) value in a Eotaxin-3 release assay of less than 100 micromolar, or of less than 10 micromolar, or less than 100 nanomolar, or less than 10 nanomolar

In one or more embodiments of the present disclosure, the compounds of general formula (I) have an (K_{D}) value in a Surface plasmon resonance (SPR) assay of less than 10 micromolar, or of less than 1 micromolar, or less than 100 nanomolar.

The compounds of the disclosure may be obtained in crystalline form either directly by concentration from an organic solvent or by crystallisation or recrystallisation from an organic solvent or mixture of said solvent and a co-solvent that may be organic or inorganic, such as water. The crystals may be isolated in essentially solvent-free form or as a solvate, such as a hydrate. The disclosure also provides crystalline forms of compounds of the disclosure, such as polymorphs and pseudopolymorphs, and also mixtures thereof.

Compounds of the disclosure comprise asymmetrically substituted (chiral) carbon atoms which give rise to the existence of isomeric forms, e.g. enantiomers and diastereomers, and other stereoisomeric forms that can be defined, in terms of absolute stereochemistry, as (R)— and (S)—. The present disclosure includes all such isomers, either in optically pure form or as mixtures thereof (e.g. racemic mixtures or partially purified optical mixtures). Pure stereoisomeric forms of the compounds and the intermediates of this disclosure may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occur stereoselectively or stereospecifically. For example, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the disclosure. When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diasterometically or enantiomerically enriched form. Where chirality is not specified but is present, it is understood that the embodiment is directed to either the specific diasteromerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposeable mirror images of one another.

“Diastereomers” are stereoisomers that have at least two asymmetric atoms, but which are not mirror-images of each other.

“STAT6 modulator” refers to compounds of the present disclosure that inhibit or reduce some or all of the activity of the Signal transducer and activator of transcription 6 (STAT6), including STAT6 degraders.

“STAT6 degrader” refers to compounds of the present disclosure that bind to and/or inhibit both STAT6 protein and an E3 ligase with measurable affinity, resulting in the ubiquitination and subsequent degradation of the STAT6 protein. In certain embodiments, a STAT6 degrader has an DC50 of less than about 50 pM, less than about 1 pM, less than about 500 nM. less than about 100 nM, less than about 10 nM, or less than about 1 nM. As used herein, the term “monovalent” refers to a degrader compound without an appended E3 ligase binding moiety.

STAT6-mediated disorders, diseases, and/or conditions refers to any disease or other deleterious condition in which STAT6 or a mutant thereof, are known to play a role.

A “subject” or “patient” is meant to describe a human or vertebrate animal, including a dog, cat, horse, cow, mouse, or the like.

“Treatment” or “treating” is an approach for obtaining beneficial or desired results including clinical results, such as inhibiting the disease or condition (e.g., decreasing one or more symptoms resulting from the disease or condition, and/or diminishing the extent of the disease or condition), slowing or arresting the development of one or more clinical symptoms associated with the disease or condition, and/or relieving the disease, enhancing the effect of another medication, delaying the progression of the disease, increasing quality of life, and/or prolonging survival.

In the compounds of formula (I), the atoms may exhibit their natural isotopic abundances, or one or more of the atoms may be artificially enriched in a particular isotope having the same atomic number, but an atomic mass or mass number different from the atomic mass or mass number found in nature. The present disclosure includes all suitable isotopic variations of the compounds of formula (I). For example, different isotopic forms of hydrogen include ¹H, ²H and ³H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regimens, or may provide a compound useful as a standard for characterization of biological samples. Isotopically enriched compounds within formula (I) can be prepared by conventional techniques well known to a person skilled in the art or by processes analogous to those described in the general procedures and examples herein using appropriate isotopically enriched reagents and/or intermediates.

In some embodiments, compounds described herein, or pharmaceutically acceptable salts, isomers, or a mixture thereof, have from 1 to n hydrogen atoms attached to a carbon atom replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

In some embodiments, a compound of the disclosure is a solvate or a hydrate.

In one embodiment, the disclosure provides a compound as above for use in therapy.

In a further embodiment, the disclosure relates to a compound as above for use in the treatment of a disease, disorder or condition, which disease, disorder or condition is responsive of modulation of STAT-6.

In a further embodiment, the disclosure relates to a compound as above for use in the treatment of autoimmune diseases.

The compounds of the present disclosure may be useful for preventing, treating or ameliorating diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides the use of a compound of formula (I) as defined above, in the manufacture of a medicament for the prophylaxis, treatment or amelioration of any of the following diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

In an embodiment the disclosure provides a method of preventing, treating or ameliorating diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

In an embodiment the disclosure provides a method of preventing, treating or ameliorating autoimmune diseases, conditions characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors the method comprising administering to a person suffering from at least one of said diseases an effective amount of one or more compounds according to formula (I), optionally together with a pharmaceutically acceptable carrier or one or more excipients, optionally in combination with other therapeutically active compounds.

Besides being useful for human treatment, the compounds of the present disclosure may also be useful for veterinary treatment of animals including mammals such as horses, cattle, sheep, pigs, dogs, and cats.

### Pharmaceutical Compositions and Modes of Administration

For use in therapy, compounds of the present disclosure are typically in the form of a pharmaceutical composition. In some embodiments, a pharmaceutical composition comprising a compound of Formula (I), optionally together with one or more other therapeutically active compound(s), together with a pharmaceutically acceptable excipient, vehicle or carrier(s). In some embodiments, the excipient is “acceptable” in the sense of being compatible with the other ingredients of the composition and not deleterious to the recipient thereof.

In some embodiments, the compound of the disclosure is provided in an amount of 0.0001-99.9% by weight of a formulation.

In the form of a dosage unit, a compound of the disclosure may be administered one or more times a day at appropriate intervals. In one embodiment, a dosage unit of a formulation contain between 0.001 mg and 1000 mg, such as between 0.01 mg and 300 mg of a compound of Formula (I).

A suitable dosage of the compound of the disclosure may depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In some embodiments, a single dose comprising a compound of the disclosure may provide an amount of the compound in the range from 0.001 to 400 mg/kg body weight.

In some embodiments, a compound of the disclosure is provided in combination with one or more therapeutically active compounds. If the treatment involves administration of another therapeutically active compound Goodman & Gilman's The Pharmacological Basis of Therapeutics, 9^{th} Ed., J. G. Hardman and L. E. Limbird (Eds.), McGraw-Hill 1995, may be consulted for useful dosages of said compound.

The administration of a compound of the present disclosure with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations may be prepared by uniformly and intimately bringing the active ingredient into association with a liquid carrier, semisolid carrier or a finely divided solid carrier or combinations of these, and then, if necessary, shaping the product into the desired formulation.

Formulations of the present disclosure suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

In some embodiments, administration can be topical (including transdermal, epidermal, ophthalmic and to mucous membranes including intranasal, vaginal and rectal delivery).

In some embodiments, pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

In some embodiments, one or more compounds as disclosed herein or a pharmaceutical composition thereof is formulated for topical administration to the skin or mucosa (e.g., dermally or transdermally).

In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

### Combination Therapies

In one embodiment, the compounds disclosed herein may be used in combination with one or more additional therapeutic agents. In some embodiments, a compound of the present disclosure, or a pharmaceutically acceptable salt or stereoisomer thereof, can be combined with the therapeutically effective amount of one or more (e.g., one, two, three, four, one or two, one to three, or one to four) additional therapeutic agents.

In some embodiments, a compound of the disclosure, or a pharmaceutically acceptable salt or stereoisomer thereof, is co-administered with one or more agents useful for the treatment and/or prophylaxis of an inflammatory, and/or dermatologic disease or disorder, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), cyclosporine, methotrexate, mycophenolate mofetil, azathioprine, interferon gamma, phosphodiesterase 4 (PDE4) inhibitor such as crisaborole, JAK inhibitor (e.g., ruxolitinib, upadacitinib, abrocitinib, baricitinib), dupilumab, and anti-IL-13 antibody (e.g., tralokinumab).

In some embodiments, a compound of the disclosure, or a pharmaceutically acceptable salt or stereoisomer thereof, is co-administered with one or more agents useful for the treatment and/or prophylaxis of a dermatologic condition, such as acne. Non-limiting examples of such agents include topical therapies such as benzoyl peroxide, topical retinoids, topical antibiotic, clascoterone, salicylic acid and azelaic acid; and systemic therapies such as doxycycline, minocycline, sarecycline, combined oral contraceptives, spironolactone, and isotretinoin.

In some embodiments, a compound of the disclosure, or a pharmaceutically acceptable salt or stereoisomer thereof, is co-administered with one or more agents useful for the treatment and/or prophylaxis of a dermatologic condition, such as alopecia areata. Non-limiting examples of such agents include topical therapies such as systemic corticosteroids (such as prednisolone), cyclosporine, azathioprine, methotrexate, sulfasalazine, simvastatin/exetimibe, inosiplex, antihistamines (such as fexofenadine), and oral JAK inhibitors (such as ritlecitinib or brepocitinib).

In some embodiments, a compound of the disclosure, or a pharmaceutically acceptable salt or stereoisomer thereof, is co-administered with one or more agents useful for the treatment and/or prophylaxis of a dermatologic condition, such as asthma. Non-limiting examples of such agents include inhaled ICS-formoterol (such as budesonide-formoterol), short-acting beta2 agonists (such as albuterol sulfate), leukotriene receptor antagonists (such as montelukast), immunoglobulin E antibodies (such as omalizumab) and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic obstructive pulmonary disease (COPD). Non-limiting examples of such agents include short-acting beta2 agonists (such as albuterol sulfate), short-acting muscarinic antagonists (such as aiprtropium), long-acting beta2 agonists (such as olodaterol), and long-acting muscarinic antagonists (such as tiotropium).

In some embodiments, a compound of the disclosure, or a pharmaceutically acceptable salt or stereoisomer thereof, is co-administered with one or more agents useful for the treatment and/or prophylaxis of a dermatologic condition, such as chronic rhinosinusitis with polyps. Non-limiting examples of such agents include intranasal corticosteroid, or biologics such as benralizumab (targets IL-5), dupilumab (targets IL-13), omalizumab (targets IgE).

In some embodiments, a compound of the disclosure, or a pharmaceutically acceptable salt or stereoisomer thereof, is co-administered with one or more agents useful for the treatment and/or prophylaxis of a dermatologic condition, such as contact dermatitis. Non-limiting examples of such agents include topical corticosteroids, topical calcineurin inhibitors (such as pimecrolimus, tacrolimus), topical phosphodiesterase 4 inhibitors, such as crisaborole, systemic immunosuppressants and modulators, such as systemic corticosteroids, methotrexate, azathioprine, mycophenolate mofetil, cyclosporine or dupilumab.

In some embodiments, a compound of the disclosure, or a pharmaceutically acceptable salt or stereoisomer thereof, is co-administered with one or more agents useful for the treatment and/or prophylaxis of a dermatologic condition, such as dermatomyositis. Non-limiting examples of such agents include topical corticosteroids, topical calcineurin inhibitors (such as pimecrolimus, tacrolimus), systemic corticosteroids (such as prednisone), antimalarials (such as hydroxychloroquine, cholorquine, quinacrine), methotrexate, mycophenolate mofetil, intravenous immunoglobulin, rituximab, and JAK inhibitors (such as tofacitinib) In some embodiments, a compound of the disclosure, or a pharmaceutically acceptable salt or stereoisomer thereof, is co-administered with one or more agents useful for the treatment and/or prophylaxis of a dermatologic condition, such as esophageal eosinophilia. Non-limiting examples of such agents include systemic corticosteroids (such as budesonide, fluticasone, prednisone), topical corticosteroids, and proton pump inhibitors (such as omeprazole, esomeprazole, pantoprazole and lansoprazole).

In some embodiments, a compound of the disclosure, or a pharmaceutically acceptable salt or stereoisomer thereof, is co-administered with one or more agents useful for the treatment and/or prophylaxis of a dermatologic condition, such as psoriasis. Non-limiting examples of such agents include topical treatments such as topical corticosteroids (such as betamethasone dipropionate, clobetasol propionate, desoximetasone, diflorasone diacetate, fluocinonide, flurandrenolide, halobetasol propionate, amcinonide, mometasone furoate, triamcinolone acetonide, fluticasone propionate, hydrocortisone valerate, clocortolone pivalate), topical calcineurin inhibitors (such as pimecrolimus, tacrolimus), topical vitamin D analogues (such as calcipotriene, calcitriol, tacalcitol or mazacalcitol), topical retinoids (such as tazarotene); systemic nonbiologic therapies such as methotrexate, phosphodiesterase 4 inhibitors (such as apremilast), immunosuppressants (such as cyclosporine), oral retinoids (such as acitretin), oral Janus kinase inhibitors (such as tofacitinib), fumaric acid esters (such as dimethyl fumarate), systemic immunosuppressants and antimetabolites (such as hydroxyurea, mycophenolate mofetil, azathioprine, leflunomide, tacrolimus and thioguanine); and biologic therapies such as TNF-a inhibitors (such as etanercept, infliximab, adalimumab, certolizumab), IL-12/IL-23 inhibitors (such as ustekinumab), IL-17 inhibitors (such as secukinumab, ixekizumab, brodalumab), and IL-23 inhibitors (such as guselkumab, tildrakizumab, risankizumab).

In some embodiments, a compound of the disclosure, or a pharmaceutically acceptable salt or stereoisomer thereof, is co-administered with one or more agents useful for the treatment and/or prophylaxis of a dermatologic condition, such as scleroderma. Non-limiting examples of such agents include immunosuppressive treatments (such as methotrexate, mycophenolate mofetil, cyclophosphamide, tocilizumab, and rituximab), and autologous haematopoietic stem cell transplantation.

In some embodiments, a compound of the disclosure, or a pharmaceutically acceptable salt or stereoisomer thereof, is co-administered with one or more agents useful for the treatment and/or prophylaxis of a dermatologic condition, such as vitiligo. Non-limiting examples of such agents include topical treatments such as topical corticosteroids, topical calcineurin inhibitors (such as pimecrolimus, tacrolimus), topical vitamin D analogues (such as calcipotriene); and systemic therapies such as oral corticosteroids (such as betamethasone).

In some embodiments, an additional therapeutic agent includes one or more of 608, 610, 611, clindamycin phosphate+benzoyl peroxide, 101BHG-D01, 1-H-11, 4P-022, 5-OXO-ETE receptor antagonists, 9MW-1911, AB-1000, AB-101a, abatacept, ABBV-712, ABCL-575, Ab-IPL-IL-17, ABM-125, abrocitinib, ABY-035/AFO2, ABY-062, AC-201, ACE-1334, acitretin, aclidinium bromide, aclidinium bromide+formoterol fumarate, acumapimod, AD-17002, adakitug, adalimumab, adapalene, adapalene+benzoyl peroxide, adapalene+clindamycin hydrochloride, adapalene+clindamycin phosphate, aderamastat, Adi, ADi-100, Adipocell, adipose tissue-derived mesenchymal stem cell-derived exosomes, adipose-derived stem cell therapy, AD-MSC-CM, ADSTEM, ADX-246, aerosolized hydroxychloroquine, afamelanotide, AJ-101, AJ-303, AK-101, AK-119, AKP-08, albuterol sulfate, ALD-R491, alefacept, Allergovac depot, allogeneic adipose-derived mesenchymal stem cell therapy, allogeneic adult pluripotent stem cells, allogeneic mesenchymal stem cell therapy, allogeneic UC-MSC therapy, allogeneic umbilical cord mesenchymal stem cell therapy, AlloRx, alprazolam, AM-1476, ambroxol hydrochloride, AMG-0101, Amilo-5MER, aminolevulinic acid, aminolevulinic acid hydrochloride, aminopterin, amlitelimab, AMTX-100, AMTX-100 CF, Anapsos, ANB-032, ANB-101, anifrolumab, anti-CD19 CAR T cell therapy, anti-CD7 CAR T-cell therapy, anti-EMAP II fully humanized antibodies, anti-IL-4/IL-13 vaccine, anti-P2X7 monoclonal antibody humanized, anti-PAR2 therapeutics, antroquinonol, APD-588, APG-222, APG-777, APG-808, APG-990, APGT-001, APIRx-1603, apremilast, aprepitant, APT-101, AQ-001S, AQ-280, AR-100DP1, AR-110, arformoterol, ARG-201, ARGX-118, ARN-4079, ARO-MUC5AC, ARO-RAGE, ARO-TSLP, ARQ-234, arsenic trioxide, ARTS-011, AS-012, asengeprast, asivatrep, ASN-008, astegolimab, AT-004, AT-005, AT-0287, AT-193, ATB-1606, ATI-2138, ATL-105, ATR-006, ATR-01, ATTO-002, ATTO-1310, atuliflapon, AUR-101, auremolimab, autologous leukocyte cell therapy, avenciguat, AVI-3307, AVID-200, AVX-001, AWEPO-003, AX-158, AX-202, AZD-0284, AZD-0449, AZD-8630, azelaic acid, azelastine, azithromycin, B-244, Bacmune, bambuterol, baricitinib, barzolvolimab, BAT-6026, bazlitoran, BB-1511, BBACN, BBI-03, BBI-6000, BCG polysaccharide+nucleic acid, BCI-332, beclometasone dipropionate+formoterol fumarate, beclomethasone dipropionate, beclomethasone dipropionate+formoterol fumarate+glycopyrronium bromide, bedoradrine, begelomab, belimumab, belumosudil, bempikibart, bencycloquidium bromide, benralizumab, benzoyl peroxide, benzoyl peroxide+tretinoin, berdazimer sodium, bermekimab, bersiporocin dihydrochloride, bertilimumab, betamethasone, betamethasone dipropionate, betamethasone valerate, bexotegrast, BFP-002, BFP-102, BGB-23339, BI-1291583, BI-1323495, BI-765250, bilastine, bimekizumab, bimiralisib, BIO-11006 Inhalation Solution, BioLexa, BITT-CD4D11, BITT-CD4F10, BLR-200, BLU-808, BMS-986313, BMS-986322, BMS-986326, BMX-010, boningmycin, BOS-475, Bosakitug, bosentan, bovhyaluronidase azoximer, Box-5, BR-201, branebrutinib, BRE-AD01, brensocatib, brentuximab vedotin, brepocitinib, brilacidin, brilaroxazine hydrochloride, briquilimab, brodalumab, BSI-056T, BSI-502, BTX-1204, BTX-1308, BTX-1503, budesonide, budesonide+arformoterol, budesonide+formoterol, budesonide+formoterol fumarate, budesonide+procaterol hydrochloride, budesonide+salbutamol, budesonide+salmeterol, buloxibutid, BV-200 series, BVX-20, BZ-371, BZ-371B, C4X-6746, C-867, CABA-201, CAL-4, calcipotriol, calcipotriol+betamethasone, calcipotriol+betamethasone dipropionate, calcipotriol+cortisone, calcitriol, CALY-002, camoteskimab, CAN-10, cannabidiol, cannabidiol+dronabinol, cannabinoid CB2 receptor agonist antibody, carbon dioxide+perfluorooctyl bromide, cavosonstat, CB-06-01, CB2 receptor agonists, CB5138-3, CC-90006, CC-92252, CCI-15106, CCX-624, CD19-CAR-DNT, CEE-321, cendakimab, certolizumab pegol, CG-459, Chanllergen, CHF-6333, CHF-6366, CHF-6550, ciclesonide, ciclosporin, ciprofloxacin hydrochloride, CIT-013, CJRB-402, CKBA, clascoterone, CLBS-03, clindamycin, clindamycin phosphate+benzoyl peroxide, clindamycin phosphate+tretinoin, clobetasol propionate, clobetasol propionate+tretinoin, CM-101, CM-326, CMK-389, CMR-316, CMS-D001, ColiFin, COPD vaccine, cord blood derived stem cells, corticotropin, COYA-204, CPL-409116, crisaborole, CS-12192, CS-32582, CS-43001, CSJ-117, CSPCHA-115, CT-05, CT-303, CT-P55, CTX-101, CTXT-102, cudetaxestat sodium, CUR—N399, Cutaquig, CVXL-0074, CXF-11, CXG-86, CXG-87, cyproterone acetate+ethinyl estradiol, D-2570, D4-103-01, D4-103-02, D4-103-03, D4-103-04, daniluromer, dapansutrile, dapsone, daridorexant hydrochloride, daxdilimab, dazukibart, DB-007-4, DBI-001, DBM-1152A, DC-806, DC-853, deflazacort, delgocitinib, denifanstat, depemokimab, dersimelagon, desloratadine, desogestrel+ethinylestradiol, desonide, deucravacitinib, deuruxolitinib phosphate, dexamethasone sodium phosphate, dexpramipexole, difamilast, dimethyl fumarate, dimethyl fumarate+ethyl hydrogen fumarate calcium+ethyl hydrogen fumarate magnesium+ethyl hydrogen fumarate zinc, diroleuton, dithranol cream, divozilimab, DLQ-02, DLX-105, DLX-2323, DMT-210, DMT-310, DMX-700, DMXD-011, DNX-114, doxofylline, doxofylline (bronchiectasis), Alitair Pharmaceuticals, doxycycline hyclate, doxycycline hyclate (delayed release), Mayne, doxycycline hyclate (easy-to-swallow, acne, bacterial infection), Aqua Pharmaceuticals, DPT-0218, drospirenone+ethinylestradiol, dual alpha-V/beta-1 and alpha-5/beta-1 integrin inhibitors, dual AMCase/CHIT1 inhibitors, dual anti-CD19/anti-BAFF CAR T-cell therapy, dual JAK3/TEC inhibitor, dupilumab, dust mite vaccine, DW-2008S, DYV-024, DZ-2002, EB-005, EB-06, EBI-H, eblasakimab, efzofitimod, EI-001, elapegademase, elarekibep, emedastine, empasiprubart, ENA-002, ENB-109, endonuclease modulators, ENERGI-F708, enpatoran, ensifentrine, ensifentrine+glycopyrrolate, EP-104-GI, EP-262, epeleuton, Epi-13, epinastine hydrochloride, epinephrine, EpiTight, EPM-301, EQ-101, erdosteine, erlotinib, ESK-001, etanercept, EtanerRel, ETD-001, ETH-47, etrasimod, etrinabdione, EVX-B4, EYD-001, F-200, F-528, factor D inhibitor, farudodstat, FB-102, FB-401, FB-704A, FB-825, FB-918, FCR-001, FCX-013, fevipiprant, filgotinib maleate, fipaxalparant, flunisolide, fluocinonide, fluticasone, fluticasone+formoterol, fluticasone furoate, fluticasone furoate+umeclidinium+vilanterol, fluticasone furoate+vilanterol trifenatate, fluticasone propionate, fluticasone propionate+formoterol fumarate, fluticasone propionate+salbutamol sulfate, fluticasone propionate+salmeterol, fluticasone propionate+salmeterol xinafoate, formoterol, formoterol fumarate, formoterol fumarate+fluticasone propionate, formoterol fumarate+glycopyrronium bromide, FPP-003, FPP-005, froniglutide, FRTX-02, FTC-001, FWB-1313, FZ-007, FZJ-003, GABAA receptor agonists, Gamunex, GB-001, GB-0895, GD-134, GD-iExo-001, gefurulimab, GEN-501, GL-7190, GLPG-3667, glutathione+ascorbic acid+bicarbonate, glycopyrrolate+formoterol fumarate+budesonide, glycopyrronium+formoterol fumarate+fluticasone propionate, glycopyrronium+vilanterol, glycopyrronium bromide, glycopyrronium bromide+indacaterol maleate, GMDP, GM-XANTHO, GN-037, GNKS-356, GNR-068, GPCR antagonists, GR-010, GR-1501, GR-1802, GR-2002, GR-2301, Grastek, GRC-39815, GRT-6015, GSK-1070806, GSK-2831781, GSK-3862995B, GSK-3923868, GT-20029, gumokimab, gusacitinib, guselkumab, GZ-21T, H-018, halobetasol propionate, halobetasol propionate+tazarotene, halogenated xanthene, halometasone, HB00-17, HB-0034, HB-0043, HB-1734, HBM-9001, HBM-9378, HCW-9302, HDM-3010, HECB-1800301, HEMP-001, HI-1640V, histamine human immunoglobulin, Hizentra, HJ-787, HL-231, HLA-open conformer-specific monoclonal antibody, HLK-6002, house dust mite allergen, house dust mites immunotherapy, HP-1901, Hpb glutamate dehydrogenase modulator, HPP-737, HpVac-R13, HRG-2005, HRS-9821, HS-10374, HS-401, HT-004, HuL-001, human adipose-derived mesenchymal stem cells, human umbilical cord-derived mesenchymal stem cell therapy, HY-07170702, HY-072808, HY-1770, HY-209, hypericin, hypochlorous acid, HZ-J001, IBI-3002, IBI-356, IBIO-100, IBL-101, icanbelimod, ICP-332, ICP-488, iCP-NI, ifetroban, IFNalpha kinoide, IgE inhibitors, IHL-675A, IL-17 NanoAb, IL-25 targeted therapeutic, IL-4R alpha antagonist, IL-4Ra targeted therapeutic, ILB-2107, iloprost, IMB-101, IMG-007, IMG-008, IMG-036, immune globulin intravenous, imsidolimab, IMX-120, IN-A002, inaticabtagene autoleucel, INCB-054707, indacaterol, indacaterol acetate+glycopyrronium bromide+mometasone furoate, Indamet, inebilizumab, infliximab, Integrin alpha-2/beta-1 inhibitor, Integrin alpha-5/beta-1 inhibitor, Interleukin IL-17A inhibitor, INV-007, INV-103, INV-17, IPG-1094, IPG-7236, ipratropium+fenoterol, ipratropium bromide, ipratropium bromide+salbutamol sulfate, IR-444, IRL-201104, IRX-4204, isotretinoin, itepekimab, itolizumab, ivacaftor, ivarmacitinib sulfate, ivermectin, ixekizumab, izokibep, JadiCell therapy, JAK inhibitors, JAK-989, jaktinib dihydrochloride monohydrate, jaktinib hydrochloride, JK-0001, JK-0002, JNJ-1459, JNJ-2113, JNJ-3534, JNJ-67484703, JRF-106, JRF-401, JRP-878, JS-005, JTE-051, JTE-451, JW-1601, JW-202232, JW-2202, JYB-1904, JYP-0061, JYP-0066, K-1032, KB-5XX, KBL-693, KBL-697, KI-696, KINE-201, KITCL-27, KN-002, KP-470, KT-294, KT-474, KT-621, KX-826, KYV-101, L-608, LABA+LAMA therapy, Langopept, larsucosterol, LAS-200019, LBG-1600M, LCK inhibitor, lebrikizumab, lepzacitinib, levalbuterol, levalbuterol hydrochloride, levonorgestrel+ethinylestradiol, LG-283, LGM-1506, LGM-2605, LH-8, LIT-00505, lithium succinate, LMY-920, LNK-01001, LNK-01004, LNP-1955, LNR-653.1, londamocitinib, long acting beta agonist/long acting muscarinic agonist, long-acting aerosolized peptide-based therapy, lonodelestat acetate, lp-003, LP-0200, LQ-036, LQ-041, LQ-043, LR-19019, LR-20016, LT-002-158, lucinactant, lunsekimig, LUT-014, LW-104, LY-3509754, LY-3872386, LY-3972406, LYS-006, lysophospholipase inhibitor, LZM-012, M-119102, M3 muscarinic receptor antagonists, M-605110, M-610101, manfidokimab, masitinib, MAX-40070, maxacalcitol, maxacalcitol+betamethasone, MCM-001, MDI-1228, MDNA-413, MDPK-67b, ME-3183, Melgain, mepolizumab, mesalazine, Mesenchymal stem/stromal cell therapy, mesenchymoangioblast-derived mesenchymal stem cell therapy, metenkefalin acetate+tridecactide acetate, methotrexate, methyl aminolevulinate hydrochloride, methylprednisolone suleptanate, MG-01, MG-K10, MG-S-2525, MGY-1838, MG-ZG122, MH-004, MH-080, minocycline, minocycline+adapalene, minocycline hydrochloride, MIT-001, mitiperstat, Mitizax, MM-09, mometasone, mometasone+formoterol, mometasone furoate, mometasone furoate+indacaterol acetate, monlunabant, montelukast, montelukast sodium, montelukast sodium+levocetirizine dihydrochloride, mosedipimod, mouse monoclonal antibody against human interleukin-8, MP-1032, MSB-01, MSB-03, MSB-3163, MSM-605, MT-5562, MTC-896, mucosa-associated lymphoid tissue lymphoma translocation protein 1 inhibitors, mufemilast, MufroSyn, mugwort pollen allergen vaccine, muscarinic M3 receptor antagonist, MYJ-1633, nacystelyn, nadifloxacin, nadolol, nalfurafine, NBL-012, NCP-111, NCP-112, ND-003, NDX-3315, NDX-3324, nedocromil, nemolizumab, netakimab, nibrozetone, niclosamide, NIK inhibitors, nitric oxide, nitroglycerin, NLP-91, NM26-2198, nomacopan, norethindrone acetate+ethinylestradiol, noscapine/noscapine analogs, NP-339, nrf2 activator, NS-402, NTR-441, NVS-451, NX-73, OATD-01, OB-756, obefazimod, OC-701, OCR-4715, Octagam 10%, olodaterol, olodaterol hydrochloride+tiotropium bromide monohydrate 1, olopatadine, OLX-103, OM-001, omalizumab, omiganan pentahydrochloride, omilancor, OMN-71, ONO-4685, OP-2101, opinercept, OpSCF, ordesekimab, ORI-001, orismilast, ORKA-001, ORKA-002, orticumab, ozagrel hydrochloride, ozenoxacin, PA-9159, paridiprubart, PBF-680, PBI-100, PC-114, PDC-APB, PDE4 inhibitor, pegtarazimod, pemirolast, peresolimab, PF-07264660, PF-07275315, PG-011, PG-102, Viromed, phimelanotide, PHP-1212, PI3K-delta inhibitor, picankibart, piclidenoson, pimecrolimus, PIPE-791, pirfenidone, pitavastatin, PKC theta inhibitors, PLM-301, PNV-5032, POLB-002, ponesimod, potassium dobesilate, PR-023, pranlukast, pranlukast hydrate, PRCL-02, PrEP-001, prostaglandin D2 synthase inhibitors, Prozumab, PRP-PBMC autologous cellular therapy, PS-35, psoriasis therapeutics, PT-101, P-TET, PUL-042, PUR-0110, PUR-1800, PX-128, PX-130, PZ-07/2024, Q-1804, Q-301, QBKPN, QLM-3003, QN-02, QP-CO1, QRX-008, quisovalimab, QX-002-N, QX-004-N, QX-005-N, QX-007-N, QX-008-N, QX-009-N, QX-010-N, QY-101, QY-201, QY-211, R-187, R-552, rademikibart, rare phytocannabinoids, ravulizumab, RAY-121, RB-1000, RBM-009, RBN-012759, RBO-0987, RC-1416, RCD-405, recombinant midismase, reformulated calcipotriol+betamethasone, REGEND-001, REGN-1908-1909, remetinostat, remibrutinib, renzapride, repirinast, repurposed aldesleukin, Repurposed azeliragon, repurposed lenabasum, reslizumab, RESP-1000 series, RESP-2000 series, RESP-X, retinoic acid, revefenacin, REX-7117, rezpegaldesleukin, RG-6151, RG-6244, RG-6314, RG-6315, RG-6341, RG-6421, RGRN-305, rilzabrutinib, riociguat, risankizumab, ritlecitinib, rituximab, RLS-1496, RLV-102, rocatinlimab, roflumilast, ropsacitinib, ROR gamma T inverse agonists, ROR-gamma inverse agonists, rose bengal sodium, RP-3128, RSBT-001, RSS-0393, R-TPR-022, rupatadine+montelukast, RUTI, ruxolitinib, RYSW-01, S1P1 agonist, salbutamol, salmeterol, salmeterol xinafoate+fluticasone propionate, SAMiRNA program, SAR-441566, SAR-443726, sarecycline, SB-010, SB-011, SCD-044, SCD-153, SCT-640A, SCT-650-C, SDC-1801, secukinumab, SEGRA, seletalisib, SEL-K2, selnoflast, seratrodast, SFA-002, SFA-004, SG-100, SGT-510, SH2 domain inhibitor program targeting STAT6, SHR-1703, SHR-1819, SHR-1905, SHR-4597, si-544, SIG-1322, SIG-1451, SIG-1456, SIM-0278, SIM-335, sitaxentan, SKI—O-703, SKL-XYZ, SLS-008, SM-17, small mobile stem cell therapy, SMET-D1, SNC-103, SNG-001, SNG-100, SNK-01, sodium chromoglycate, sodium pyruvate, sonelokimab, soquelitinib, sovleplenib, spesolimab, SPL84-23, SSGJ-621, SSS-07, ST-1830, stapokibart, STAR-0310, STAT3 inhibitor, STMC-103H, STS-01, STSA-1201, SUDO-286, SUL-238, SuperMApo, suplatast tosilate, SYHX-1901, SYX-5219, T-517, tacalcitol, tacrolimus, TAF-001, tagraxofusp, TAGX-0003, TAKC-02, tanimilast, TAP-1502, TAP-1503, tapinarof, Tavo-101, tazarotene, tazarotene+betamethasone dipropionate, tazarotene+clindamycin, TD-8236, TDM-180935, TDM-Atop01, TDM-Psor01, TDM-Scar01, telazorlimab, Tempol, temtokibart, teprotumumab, TER-101, terbutalin, terguride, tesnatilimab, TEV-48574, TEV-53275, tezepelumab, TFF-HMW-HA, Thalassophryne nattereri peptide, THB-001, theophylline, THOR-809, TI-520, TI-620, tibulizumab, tildrakizumab, timolumab, tiotropium, tiotropium bromide, tipelukast, tirbanibulin, TLL-018, TLY-012, TO-210, tofacitinib, tofacitinib+fingolimod, tofacitinib citrate, tonabacase, TOP-N44, TOP-N53, torudokimab, tosufloxacin, tozorakimab, TP-317, TQC-2731, TQC-2938, TQC-3564, TQC-3721, TQC-3927, TQH-2722, TQH-2929, TQH-3906, TQH-3910, trabikibart, tralokinumab, tranilast, transcription factor pathway inhibitor, tregalizumab, treprostinil, treprostinil diolamine, tretinoin, tretinoin+benzoyl peroxide, trifarotene, TRIV-509, TRN-157, TRPA1 antagonists, TS-0001, TT-01, TT-01688, tulinercept, tulobuterol, TVB-3567, UA-021, UB-221, UCB-1381, UCB-9741, ucenprubart, UHE-101, UHE-105, UI-009, UI-010, UI-031, UI-033, UI-034, ulobetasol, umbilical cord blood-derived stem cell therapy, UMC119-06, umeclidinium bromide, umeclidinium bromide+vilanterol trifenatate, upadacitinib, USP-4 inhibitors, ustekinumab, UTAA-09, VALERGEN-DS, vamorolone, vapendavir, vardenafil, VB-1953, VC-005, VDAA, VDAD, VDJ-006, VEGFR targeted DK4/10, venanprubart, VENT-03, verekitug, vilanterol+fluticasone furoate+glycopyrronium bromide, vipoglanstat hydrogensulfate, VISTA agonist, vixarelimab, VLRX-001, VM-AD, VNLG-152, vonifimod, voriconazole, votucalis, VRN-04, VS-105, VSG-158, VSTM-1 agonist, VT-014, VTH-212, vunakizumab, VYN-201, VYN-202, W16P-0576, WD-890, WM-1R3, WNT inhibitor, WNT pathway agonist antibodies, wnt pathway stimulator, WXFL-10203614, WXSH-0150, XCUR-17, XKH-001, XmAb-564, XT-0528, XZ.700, YH-25487, YH-35324, YKRH-00020, YR-001, Yso3, zabedosertib, zafirlukast, zasocitinib, ZB-168, Zemaira, ZeP-3, zetomipzomib, zevaquenabant, ZHB-107-108, zibotentan, zileuton, zirconium zr 89 crefmirlimab berdoxam, ZL-1102, ZPL-521, and/or bi-specific antibodies targeting one or more targets referenced herein.

In some embodiments a compound of the disclosure provided herein, or pharmaceutically acceptable salt or stereoisomer thereof, is administered with one or more therapeutic agents selected from a PPARd inhibitor, IRAK4 inhibitor, TPL2 inhibitor, α4β7 inhibitor, BTLA agonist, PD1 agonist, or an FXR agonist.

In some embodiments a compound of the disclosure provided herein, or pharmaceutically acceptable salt or stereoisomer thereof, is administered with one or more therapeutic agents selected from seladelpar, edecesertib, tilpisertib fosmecarbil, GS-1427, GS-0272, GS-0151, or cilofexor.

The benefit of combination may be increased efficacy and/or reduced side effects for a component as the dose of that component may be adjusted down to reduce its side effects while benefiting from its efficacy augmented by the efficacy of the compound of the present disclosure.

In some embodiments, the additional therapeutic agent includes an agent useful for modulating, treating, or preventing inflammation, such as a 4-1BB ligand, 5-Alpha-reductase inhibitor, 5-HT 1a receptor antagonist, 5-HT 1a receptor partial agonist, 5-HT 2a receptor antagonist, 5-HT 2a receptor partial agonist, 5-HT 2b receptor antagonist, 5-HT 3 receptor antagonist, 5-HT 4 receptor agonist, 5-HT 6 receptor antagonist, 5-HT 7 receptor antagonist, 5-Lipoxygenase activating protein inhibitor, 5-Lipoxygenase inhibitor, Accessory gene regulator A inhibitor, Acetaldehyde dehydrogenase modulator, Acetylcholine receptor agonist, Acetylcholine receptor antagonist, Acetylcholinesterase inhibitor, Acidic mammalian chitinase inhibitor, ACTH receptor agonist, Activity-dependent neuroprotector modulator, ADAM-33 inhibitor, ADAM-9 inhibitor, Adenosine A1 receptor antagonist, Adenosine A1 receptor modulator, Adenosine A2b receptor antagonist, Adenosine A3 receptor agonist, Adenosine A3 receptor antagonist, Adenosine deaminase stimulator, Adenosylhomocysteinase inhibitor, Adenylate cyclase stimulator, Adrenergic receptor agonist, Adrenocorticotrophic hormone ligand, Advanced glycosylation product receptor antagonist, AGER gene inhibitor, AIMP multisynthetase complex protein 1 inhibitor, Albumin antagonist, Alcohol dehydrogenase 5 inhibitor, Aldose reductase inhibitor, Alk-5 protein kinase inhibitor, Alpha 1 antitrypsin modulator, Alpha 1 antitrypsin stimulator, Alpha 1 proteinase inhibitor, Alpha 2 adrenoceptor agonist, Amiloride sensitive sodium channel inhibitor, AMP activated protein kinase alpha 2 stimulator, AMP activated protein kinase stimulator, Amyloid protein deposition inhibitor, Androgen receptor antagonist, Angiotensin II AT-1 receptor antagonist, Angiotensin II AT-2 receptor agonist, Anoctamin 1 stimulator, Aortic smooth muscle actin inhibitor, AP1 transcription factor modulator, Apelin receptor agonist, Apolipoprotein A antagonist, Apolipoprotein A5 stimulator, Apolipoprotein B modulator, Apolipoprotein E modulator, Apoptosis regulator Bcl X inhibitor, Apoptosis regulator Bel w inhibitor, APRIL receptor modulator, Aryl hydrocarbon receptor agonist, Aryl hydrocarbon receptor modulator, B and T lymphocyte attenuator stimulator, B-lymphocyte antigen CD19 inhibitor, B-lymphocyte antigen CD19 modulator, B-lymphocyte antigen CD20 inhibitor, B-lymphocyte stimulator ligand inhibitor, B-lymphocyte stimulator ligand modulator, Bcl-2 protein inhibitor, Beta 1 adrenoceptor antagonist, Beta 2 adrenoceptor agonist, Beta 2 adrenoceptor antagonist, Beta 2 adrenoceptor modulator, Beta adrenoceptor agonist, Beta amyloid antagonist, Beta-catenin inhibitor, Beta-catenin modulator, Bifunctional aminoacyl tRNA synthetase inhibitor, BMP10 gene inhibitor, BMP15 gene inhibitor, Bone marrow proteoglycan modulator, Botulinum toxin A stimulator, Bromodomain containing protein 1 inhibitor, Bromodomain containing protein inhibitor, Btk tyrosine kinase inhibitor, C-myc binding protein inhibitor, C-type lectin domain protein 4C inhibitor, Ca2+ release activated Ca2+ channel 1 inhibitor, Calcineurin inhibitor, Calcium channel inhibitor, Cannabinoid CB1 receptor antagonist, Cannabinoid CB1 receptor inverse agonist, Cannabinoid CB2 receptor agonist, Cannabinoid CB2 receptor modulator, Cannabinoid receptor agonist, Cannabinoid receptor antagonist, Cannabinoid receptor modulator, Catalase stimulator, CCL26 gene inhibitor, CCR3 chemokine modulator, CCR5 chemokine antagonist, CCR6 chemokine antagonist, CCR8 chemokine antagonist, CD11b antagonist, CD122 agonist, CD122 modulator, CD19 modulator, CD2 antagonist, CD223 modulator, CD3 modulator, CD30 modulator, CD4 antagonist, CD40 ligand receptor antagonist, CD47 antagonist, CDw123 modulator, Cell adhesion molecule inhibitor, Cell surface glycoprotein CD200R agonist, Cell surface glycoprotein MUC18 inhibitor, CFTR modulator, CFTR stimulator, Chaperonin stimulator, Chemokine receptor antagonist, Chitinase inhibitor, Collagen I agonist, Collagen I antagonist, Collagen modulator, Collagen VII antagonist, Complement C1q subcomponent inhibitor, Complement C1s subcomponent inhibitor, Complement C5 factor inhibitor, Complement factor C2 inhibitor, Complement factor D inhibitor, COVID19 spike glycoprotein modulator, CSF-1 antagonist, CXC10 chemokine ligand inhibitor, CXCR2 chemokine antagonist, cyclic GMP AMP synthase inhibitor, Cyclooxygenase inhibitor, Cytokine receptor agonist, Cytokine receptor antagonist, Cytokine receptor common beta chain inhibitor, Cytoplasmic protein NCK inhibitor, Cytosolic phospholipase A2 inhibitor, Cytotoxic T-lymphocyte protein-4 stimulator, Deoxyribonuclease gamma stimulator, DHFR inhibitor, Diacylglycerol O acyltransferase 1 inhibitor, Dihydroorotate dehydrogenase inhibitor, Dipeptidyl peptidase I inhibitor, Dipeptidyl peptidase IV inhibitor, DNA gyrase inhibitor, DNA methyltransferase inhibitor, Dopamine D2 receptor partial agonist, Dopamine D3 receptor agonist, Dopamine D3 receptor partial agonist, Dopamine D4 receptor partial agonist, DYRK-1 alpha protein kinase inhibitor, Ectonucleotide pyrophosphatase-PDE-2 inhibitor, EGF like module receptor 1 antagonist, EGFR family tyrosine kinase receptor inhibitor, Elastase inhibitor, Endonuclease modulator, Endostatin modulator, Endothelin ET-A receptor antagonist, Endothelin ET-B receptor antagonist, Enolase 1 inhibitor, Eosinophil peroxidase inhibitor, Eotaxin 2 ligand inhibitor, Eotaxin ligand inhibitor, EP4 prostanoid receptor antagonist, Epidermal growth factor receptor antagonist, Epidermal growth factor receptor modulator, Estradiol agonist, Estrogen receptor agonist, Extracellular signal related kinase-2 inhibitor, Facilitated glucose transporter-1 modulator, Fatty acid synthase inhibitor, FGF receptor antagonist, FGF-2 ligand, FGF-4 ligand, FGF3 receptor antagonist, Filaggrin stimulator, Flt3 tyrosine kinase inhibitor, FMLP related receptor I agonist, FMLP related receptor II agonist, Free fatty acid receptor 2 agonist, Free fatty acid receptor 3 agonist, Fyn tyrosine kinase inhibitor, FXR agonist, G-protein coupled bile acid receptor 1 agonist, G-protein coupled receptor-44 antagonist, G-protein coupled receptor-44 modulator, GABA A receptor agonist, GABA A receptor alpha-2 subunit modulator, GABA A receptor alpha-3 subunit modulator, GABA A receptor alpha-5 subunit modulator, Galectin-10 modulator, GATA 3 transcription factor inhibitor, Glucagon-like peptide 1 receptor agonist, Glucocorticoid receptor agonist, Glutamate dehydrogenase modulator, Glutamate receptor modulator, Glutathione dependent PGD synthase inhibitor, Glutathione independent PGD synthase inhibitor, Glutathione reductase inhibitor, GroEL protein 2 inhibitor, Guanylate cyclase stimulator, H+K+ATPase inhibitor, Heat shock protein inhibitor, Heme oxygenase 1 modulator, Heparin agonist, High mobility group protein B1 inhibitor, Histamine H1 receptor antagonist, Histamine H4 receptor antagonist, Histamine receptor antagonist, Histone deacetylase-1 inhibitor, Histone deacetylase-2 inhibitor, Histone deacetylase-2 stimulator, Histone deacetylase-3 inhibitor, Histone deacetylase-6 inhibitor, Histone H2A modulator, Histone H4 modulator, HMG CoA reductase inhibitor, Hsp 90 inhibitor, Hsp70 binding protein 1 inhibitor, Hyaluronidase stimulator, Hypoxia inducible factor stimulator, I-kappa B kinase beta inhibitor, I-kappa B kinase epsilon inhibitor, IgG receptor FcRn large subunit p51 modulator, IL-1 receptor accessory protein inhibitor, IL-1 receptor antagonist, IL-10 receptor agonist, IL-10 receptor antagonist, IL-12 receptor antagonist, IL-13 receptor antagonist, IL-13 receptor modulator, IL-15 receptor antagonist, IL-17 antagonist, IL-18 antagonist, IL-2 receptor alpha subunit stimulator, IL-2 receptor antagonist, IL-2 receptor modulator, IL-22 antagonist, IL-23 antagonist, IL-3 receptor modulator, IL-4 receptor antagonist, IL-4 receptor modulator, IL-5 receptor antagonist, IL-6 receptor antagonist, IL-7 receptor antagonist, IL-8 receptor antagonist, IL17RA gene inhibitor, IL2 gene stimulator, Immunoglobulin E antagonist, Immunoglobulin E modulator, Immunoglobulin G agonist, Immunoglobulin G1 modulator, Immunoglobulin agonist, Immunoglobulin kappa modulator, Immunoglobulin modulator, Inducible nitric oxide synthase inhibitor, Insulin receptor substrate-1 inhibitor, Insulin-like growth factor 1 receptor antagonist, Integrin alpha-2/beta-1 antagonist, Integrin alpha-4/beta-1 antagonist, Integrin alpha-4/beta-7 antagonist, Integrin alpha-5/beta-1 antagonist, Integrin alpha-5/beta-3 modulator, Integrin alpha-V/beta-1 antagonist, Integrin beta 1 binding protein modulator, Interferon alpha 14 ligand, Interferon alpha ligand inhibitor, Interferon beta ligand, Interferon beta ligand inhibitor, Interferon gamma ligand inhibitor, Interferon gamma receptor antagonist, Interferon type I receptor antagonist, Interleukin 1 delta ligand inhibitor, Interleukin 1 like receptor (IL33R) antagonist, Interleukin 1 like receptor 1 modulator, Interleukin 1 like receptor 2 inhibitor, Interleukin 13 ligand inhibitor, Interleukin 13 receptor alpha 1 antagonist, Interleukin 15 ligand inhibitor, Interleukin 17 ligand inhibitor, Interleukin 17A ligand inhibitor, Interleukin 17A ligand modulator, Interleukin 17E ligand inhibitor, Interleukin 17E ligand modulator, Interleukin 17F ligand inhibitor, Interleukin 17F ligand modulator, Interleukin 18 ligand inhibitor, Interleukin 23A inhibitor, Interleukin 31 ligand inhibitor, Interleukin 31 ligand modulator, Interleukin 33 ligand inhibitor, Interleukin 33 ligand modulator, Interleukin receptor 17A antagonist, Interleukin receptor 17B antagonist, Interleukin-1 alpha ligand inhibitor, Interleukin-1 beta ligand modulator, Interleukin-1 ligand inhibitor, Interleukin-2 ligand, Interleukin-2 ligand inhibitor, Interleukin-31 receptor modulator, Interleukin-4 ligand inhibitor, Interleukin-5 ligand inhibitor, Interleukin-6 ligand inhibitor, Interleukin-8 ligand inhibitor, Interleukin-9 ligand inhibitor, IRAK-4 protein kinase inhibitor, IRAK-4 protein kinase degrader, Itk tyrosine kinase inhibitor, JAK tyrosine kinase inhibitor, Jaki tyrosine kinase inhibitor, Jak2 tyrosine kinase inhibitor, Jak3 tyrosine kinase inhibitor, Jun N terminal kinase inhibitor, Kallikrein 2 inhibitor, Kallikrein 5 modulator, Kallikrein 7 inhibitor, Kallikrein 7 modulator, Kallikrein inhibitor, KCNA voltage-gated potassium channel-3 inhibitor, Kelch like ECH associated protein 1 inhibitor, Kelch like ECH associated protein 1 modulator, Kit tyrosine kinase inhibitor, LanC like protein 2 stimulator, Lanosterol-14 demethylase inhibitor, Lek tyrosine kinase inhibitor, Lectin mannose binding protein inhibitor, Leukocyte Ig like receptor A4 modulator, Leukocyte elastase inhibitor, Leukotriene A4 hydrolase inhibitor, Leukotriene BLT receptor antagonist, Leukotriene C4 antagonist, Leukotriene C4 synthase inhibitor, Leukotriene D4 agonist, Leukotriene D4 antagonist, Leukotriene E4 antagonist, Leukotriene receptor antagonist, Liver X receptor agonist, LOXL2 gene inhibitor, Lung surfactant associated protein D stimulator, Lyn tyrosine kinase inhibitor, Lysophosphatidate-1 receptor antagonist, Lysophospholipase inhibitor, Macrophage migration inhibitory factor inhibitor, Major allergen I polypeptide chain 2 inhibitor, Major allergen inhibitor, MALT protein 1 inhibitor, Mannan-binding lectin serine protease inhibitor, MAP kinase modulator, MAPKAPK2 inhibitor, MARCKS protein inhibitor, Mas-related G-protein receptor X2 antagonist, Mas-related G-protein receptor X2 inhibitor, MEK protein kinase inhibitor, MEK-1 protein kinase inhibitor, Melanocortin MC1 receptor agonist, Melanocortin MC5 receptor antagonist, Melanocortin receptor agonist, Melanocyte stimulating hormone ligand, Membrane copper amine oxidase inhibitor, Metalloprotease-12 inhibitor, MEX3B gene inhibitor, Mineralocorticoid receptor antagonist, MIP 3 alpha ligand inhibitor, Mite allergen modulator, Mitochondrial 10 kDa heat shock protein stimulator, MKL myocardin like protein inhibitor, MMP1 gene stimulator, MNK protein kinase inhibitor, Monocyte chemotactic protein 1 ligand inhibitor, MS4A2 gene modulator, mTOR complex 1 inhibitor, mTOR complex 2 inhibitor, MUC5AC gene inhibitor, Muscarinic M1 receptor antagonist, Muscarinic M2 receptor antagonist, Muscarinic M3 receptor antagonist, Muscarinic M4 receptor antagonist, Muscarinic M5 receptor antagonist, Muscarinic receptor agonist, Muscarinic receptor antagonist, Muscarinic receptor modulator, Myeloperoxidase inhibitor, Myosin heavy chain inhibitor, NACHT LRR PYD domain protein 3 inhibitor, NEDD4 family interacting protein 1 stimulator, Neuropilin 2 modulator, Neutral endopeptidase inhibitor, NFE2L2 gene stimulator, Nicotinic ACh receptor alpha 7 subunit stimulator, Nicotinic acetylcholine receptor agonist, NK1 receptor antagonist, NKG2 D activating NK receptor antagonist, NLR family member X1 stimulator, Non receptor tyrosine kinase TYK2 antagonist, Nuclear erythroid 2-related factor 1 stimulator, Nuclear erythroid 2-related factor 2 stimulator, Nuclear erythroid 2-related factor inhibitor, Nuclear factor kappa B gene inhibitor, Nuclear factor kappa B inducing kinase inhibitor, Nuclear factor kappa B inhibitor, Nuclear factor kappa B modulator, Oncostatin M receptor subunit beta inhibitor, Opioid growth factor receptor agonist, Opioid receptor delta antagonist, Opioid receptor kappa agonist, Orexin 1 receptor antagonist, Orexin 2 receptor antagonist, Orphan nuclear receptor antagonist, Outer membrane protein modulator, OX-40 receptor agonist, OX-40 receptor antagonist, OX40 ligand inhibitor, OX40 ligand modulator, Oxoeicosanoid receptor 1 antagonist, P-Glycoprotein inhibitor, P-selectin glycoprotein ligand-1 inhibitor, P2X2 purinoceptor antagonist, P2X3 purinoceptor antagonist, P2X7 purinoceptor modulator, P2Y6 purinoceptor modulator, p38 MAP kinase inhibitor, p53 tumor suppressor protein stimulator, PcrV protein type III inhibitor, PDE 3 inhibitor, PDE 4 inhibitor, PDE 4b inhibitor, PDE 4d inhibitor, PDE 5 inhibitor, PDGF receptor antagonist, Peptidase 1 inhibitor, PGD2 antagonist, PGI2 agonist, Phosphatidylinositol 4 kinase beta inhibitor, Phosphoinositide 3-kinase inhibitor, Phosphoinositide-3 kinase delta inhibitor, Phospholipase A2 inhibitor, Phospholipase C inhibitor, PIM-1 protein kinase inhibitor, PIM-2 protein kinase inhibitor, PIM-3 protein kinase inhibitor, Placenta growth factor ligand inhibitor, Plasminogen activator inhibitor 1 inhibitor, Platelet activating factor receptor antagonist, Poly ADP ribose polymerase 14 inhibitor, PPAR gamma agonist, PPAR gene modulator, Progesterone receptor agonist, Programmed cell death ligand 1 modulator, Programmed cell death protein 1 modulator, Programmed cell death protein 1 stimulator, Prostaglandin E synthase inhibitor, Prostaglandin E synthase-1 inhibitor, Protease inhibitor, Protease-activated receptor-2 antagonist, Proteasome beta-8 subunit modulator, Proteasome inhibitor, Protein kinase C theta inhibitor, Protein kinase inhibitor, Protein NOV homolog modulator, Protein tyrosine kinase inhibitor, Protoporphyrinogen oxidase modulator, Pyruvate kinase muscle isozyme stimulator, Raf B protein kinase inhibitor, Ras gene inhibitor, Reactive oxygen species modulator inhibitor, Ret tyrosine kinase receptor inhibitor, Retinoic acid receptor agonist, Retinoic acid receptor antagonist, Retinoic acid receptor gamma agonist, Retinoic acid receptor gamma antagonist, Retinoic acid receptor gamma inverse agonist, Retinoic acid receptor modulator, Retinoid receptor agonist, Retinoid X receptor agonist, Retinoid X receptor modulator, Retinoid Z receptor gamma antagonist, Retinoid Z receptor gamma inverse agonist, Rev protein modulator, Rho associated protein kinase 1 inhibitor, Rho associated protein kinase 2 inhibitor, Ribonuclease P inhibitor, Ribonuclease stimulator, RIP-1 kinase inhibitor, S100 calcium binding protein A4 inhibitor, S100A8 gene inhibitor, S100A9 gene inhibitor, SARS coronavirus 3C protease like inhibitor, Secretory phospholipase A2 receptor antagonist, Serine protease inhibitor, Serine threonine protein kinase TBK1 inhibitor, Serum amyloid A protein modulator, SH2 domain containing protein inhibitor, Sialic acid-binding Ig-like lectin 8 inhibitor, SIRT3 gene stimulator, SMAD inhibitor, SNAI1 transcription factor inhibitor, SOD3 gene stimulator, Sodium channel inhibitor, Sphingosine 1 phosphate phosphatase 1 stimulator, Sphingosine-1-phosphate receptor-1 agonist, Sphingosine-1-phosphate receptor-1 antagonist, Sphingosine-1-phosphate receptor-1 modulator, Sphingosine-1-phosphate receptor-3 modulator, Sphingosine-1-phosphate receptor-4 antagonist, Sphingosine-1-phosphate receptor-4 modulator, Sphingosine-1-phosphate receptor-5 modulator, Sphingosylphosphorylcholine receptor antagonist, Src tyrosine kinase inhibitor, STAT inhibitor, STAT-1 modulator, STAT-3 inhibitor, STAT-5 inhibitor, STAT-6 inhibitor, STAT-6 degrader, Stearoyl CoA desaturase-1 inhibitor, Stress induced secreted protein 1 stimulator, Superoxide dismutase modulator, Superoxide dismutase stimulator, Syk tyrosine kinase inhibitor, Synuclein alpha inhibitor, T cell receptor antagonist, T cell receptor modulator, T cell surface glycoprotein CD28 inhibitor, T-cell antigen CD7 modulator, T-cell differentiation antigen CD6 inhibitor, T-cell surface glycoprotein CD8 inhibitor, T-cell transcription factor NFAT modulator, Tau aggregation inhibitor, Tau deposition inhibitor, Tec tyrosine kinase inhibitor, TGF beta 1 ligand inhibitor, TGF beta 3 ligand inhibitor, TGF beta 3 ligand modulator, TGF beta ligand inhibitor, TGF beta receptor agonist, TGF beta receptor antagonist, TGF-beta activated kinase-1 inhibitor, TGF-beta type II receptor antagonist, TGF-beta type II receptor modulator, TGF-beta type III receptor antagonist, Thromboxane A2 antagonist, Thromboxane synthetase inhibitor, Thymic stromal lymphopoietin ligand, Thymic stromal lymphopoietin ligand inhibitor, Thymic stromal lymphopoietin ligand modulator, Thymic stromal lymphopoietin receptor antagonist, Thymic stromal lymphopoietin receptor modulator, TLR agonist, TLR modulator, TLR-2 agonist, TLR-2 antagonist, TLR-4 antagonist, TLR-6 agonist, TLR-7 antagonist, TLR-8 antagonist, TLR-9 agonist, TLR-9 antagonist, TNF agonist, TNF alpha ligand inhibitor, TNF alpha ligand modulator, TNF antagonist, TNF binding agent, TNF related apoptosis inducing ligand, TNF-like receptor-2 modulator, Tumor necrosis factor 15 ligand inhibitor, Topoisomerase IV inhibitor, TRAIL receptor agonist, Transcription factor inhibitor, Transcription factor modulator, Transthyretin modulator, Trk tyrosine kinase receptor inhibitor, TRP cation channel A1 inhibitor, TRP cation channel A1 modulator, TRP cation channel C1 inhibitor, TRP cation channel V1 antagonist, TRP cation channel V1 modulator, TRP cation channel V2 modulator, Tsl protein kinase inhibitor, Tubulin binding agent, Tubulin receptor antagonist, Tumor necrosis factor 13B receptor modulator, Tumor necrosis factor 13C receptor modulator, Tumor necrosis factor 14 ligand inhibitor, Tumor necrosis factor 15 ligand modulator, Tumor necrosis factor ligand inhibitor, Txk tyrosine kinase inhibitor, Tyk2 tyrosine kinase inhibitor, Tyk2 tyrosine kinase modulator, Type I IL-1 receptor antagonist, Type I TNF receptor antagonist, Type II TNF receptor modulator, Tyrosine phosphatase substrate 1 inhibitor, Ubiquitin inhibitor, Ubiquitin ligase modulator, Ubiquitin ligase stimulator, Ubiquitin thioesterase-4 inhibitor, Unspecified GPCR antagonist, Unspecified GPCR modulator, Unspecified ion channel inhibitor, Uteroglobin stimulator, V-set transmembrane domain protein 1 stimulator, Vascular cell adhesion protein 1 antagonist, VEGF ligand inhibitor, VEGF receptor modulator, VEGF-2 receptor modulator, Vimentin inhibitor, Vitamin D3 receptor agonist, Wnt 5A ligand inhibitor, Wnt ligand modulator or YSK-4 protein kinase inhibitor.

### Kits

Provided herein are also kits that include a compound described herein, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of Formula (I) (or any other Formula described herein), or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### EXAMPLES

### Experimental Procedures

Intermediates were obtained sufficiently pure for the next step from the procedures outlined in the specification.

Reactions were performed at room temperature unless stated otherwise. When a compound whose synthesis is described herein was used at a larger scale than its reported synthesis, it is to be implicitly understood that more material had been prepared similarly.

Microwave reactions were performed in dedicated MW instruments in closed vials.

¹H NMR spectra were recorded at 250-600 MHz in d₆-DMSO unless stated otherwise.

Reactions performed above the boiling point of the solvent took place in sealed tubes or screw-cap vials.

Some of the final compounds (Examples) were obtained in pure form and the yield provided accordingly while others were obtained as DMSO solutions for which the concentration and volume are specified.

“Dried” refer to drying an organic solution over Na₂SO₄, MgSO₄, or CaCl₂ and filtering off the drying agent.

“Degassed” refers to air-sensitive reactions being flushed with inert atmosphere via evacuation and back-filling or by bubbling inert atmosphere through the mixture for several minutes.

NaH was used as a 60% oil dispersion.

“Filtration” of mixtures refers to the removal/isolation of solid material from mixture by filtration through a paper filter, PFTE septum, or through a pad of celite. Additional solvent was used to wash the solid.

Unless noted otherwise hydrogenations were performed at 1 bar from a balloon.

‘Purification by SCX’ refers to the loading of the material onto a SCX column, washing with MeOH, eluting with NH₃ in MeOH, and concentrating the fractions containing the product.

Catalytic hydrogenations were performed using a catalyst such as 10% Pd/C and a H₂ balloon unless stated otherwise. The mixture after the reaction was typically filtered through celite to remove the catalyst.

‘Partitioned (A/B)’ refers to the partitioning of a mixture between solvents A and B.

OL (A/B) refers to the organic layer after partitioning the mixture between solvents A and B.

AL (A/B) refers to the aq. layer after partitioning the mixture between solvents A and B.

Crude reaction mixtures after reductions with iron/zinc power and AcOH/NH₄Cl were typically filtered through celite before work-up of the filtrate.

### HPLC Methods

Several Intermediates and final compounds (Examples) were purified using the HPLC methods listed below.

| Column | Eluent | Flow rate |
|---|---|---|
| XBridge Prep C18 OBD, | A: 50 mM aq. NH₄HCO₂, B: ACN, 10-100% | 30 mL/min |
| 150 × 19 mm 5 μm | ACN | |
| XTerra ® RP-18 OBD, | A: 0.1% aq. NH₄HCO₂, B: ACN, 10-100% ACN | 30 mL/min |
| 150 × 19 mm 5 μm | | |
| PRINCETONE ULTIMA | A: 0.05% aq. HCO₂H, B: ACN | 16 mL/min |
| C18 250 × 20 mm, 5 μm | 0 min 20% B, 1 min 20% B, 10 min 70% B, 10.1 | |
| | min 99% B, 12 min 99% B, 12.1 min, 20% B, 15 | |
| | min 20% B 12.1/20, 15/20 | |
| PRINCETONE ULTIMA | A: 0.1% aq. HCO₂H, B: ACN | 16 mL/min |
| C18 250 × 20 mm, 5 μm | 0 min 20% B, 2 min 20% B, 10 min 50% B | |
| LUNA OMEGA C18 | A: 10 mM aq. NH₄HCO₂, B: ACN | 18 mL/min |
| 250 × 21.2 mm, 5 μm | 0 min 40% B, 1 min 40% B, 10 min 85% B, 10.1 | |
| | min 99% B, 12 min 99% B, 12.1 min 40% B, | |
| | 15 min 40% B | |
| COGENT C18 250 × 21.2 | A: 10 mM aq. NH₄HCO₂, B: ACN | 16 mL/min |
| mm, 5 μm | 0 min 30% B, 2 min 30% B, 10 min 50% B | |
| COGENT C18 250 × 21.2 | A: 10 mM aq. NH₄HCO₂, B: ACN | 20 mL/min |
| mm, 5 μm | 0 min 30% B, 1 min 30% B, 10 min 80% B, 10.1 | |
| | min 99% B, 12 min 99% B, 12.1 min 30% B, 15 | |
| | min 30% B | |
| LUNA C18 150 × 21.2 mm, | A: 10 mM aq. NH₄HCO₂, B: ACN | 18 mL/min |
| 5 μm | 0 min 40% B, 1 min 40% B, 10 min 80% B, 10.1 | |
| | min 99% B, 12 min 99% B, 12.1 min 40% B, 15 | |
| | min 40% B | |
| X-SELECT PHENYL | A: 10 mM aq. NH₄HCO₂, B: ACN | 16 mL/min |
| HEXYL 250 × 19 mm, 5 | 0 min 30% B, 1 min 30% B, 10 min 55% B | |
| μm | | |
| X-SELECT PHENYL | A: 10 mM aq. NH₄HCO₂, B: ACN | 18 mL/min |
| HEXYL 250 × 19 mm, 5 | 0 min 55% B, 1 min 55% B, 10 min 70% B, 15 | |
| μm | min 90% B, 15.1 min 100% B, 19 min 100% B, | |
| | 19.1 min 55% B | |
| AZZOTA 250 × 20 mm, 10 | A: 10 mM aq. NH₄HCO₂, B: ACN | 18 mL/min |
| μm | 0 min 40% B, 1 min 40% B, 10 min 85% B, 10.1 | |
| | min 99% B, 12 min 99% B, 12.1 min 40% B, 15 | |
| | min 40% B | |
| AZZOTA 250 × 20 mm, 5 | A: 10 mM aq. ABC, B: ACN | 17 mL/min |
| μm | 0 min 30% B 1 min 30% B, 10 min 85% B | |
| PRINCETONE ULTIMA | A: 0.1% aq. HCO₂H, B: ACN | 17 mL/min |
| C18 250 × 20 mm, 10 μm | 0 min 10% B, 1 min 10% B, 10 min 90% B, 10.1 | |
| | min 99% B, 13 min 99% B, 13.1 min 10% B, 16 | |
| | min 10% B | |
| XSELECT CSH C18 | A: 0.1% aq. HCO₂H, B: ACN | 17 mL/min |
| 250 × 19 mm 5 μm | 0 min 10% B, 1 min 10% B, 10 min 90% B, 10.1 | |
| | min 99% B, 13 min 99% B, 13.1 min 10% B, 16 | |
| | min 10% B | |
| YMC-PACK-ODS-AQ | A: 0.1% aq. HCO₂H, B: ACN | 15 mL/min |
| C18 250 × 20 mm 5 μm | 0 min 25% B, 2 min 25% B, 10 min 35% B | |
| HICHROME C18 250 × 20 | A: 10 mM aq. ABC, B: ACN | 18 mL/min |
| mm 5 μm | 0 min 40% B, 2 min 40% B, 10 min 80% B | |
| X SELECTC18 250 × 19 | A: 10 mM aq. ABC, B: ACN | 16 mL/min |
| mm 5 μm | 0 min 40% B, 1 min 40% B, 10 min 80% B, 11 | |
| | min 80% B, 11.1 min 99% B, 15 min 99% B, | |
| | 15.1 min 40% B, 19 min 40% B | |

The compounds of the disclosure (Examples) were characterized by the LC/MS methods listed below.

| Method | Column and eluent | Gradient | Flow rate | |
|---|---|---|---|---|
| 1 | Agilent Poroshell 120 SB-C18 4.6 × 30 | 0 min 1% B, 1.5 min | 3 | mL/min |
| | mm 2.7 μm operated at 60° C. | 100% B, 1.73 min | | |
| | A: 0.1% aq. HCO₂H | 100% B | | |
| | B: 0.1% HCO₂H in ACN | | | |
| 2 | Acquity BEH C18 50 × 2.1 mm 1.7 μm | 0 min 3% B, 0.4 min | 0.6 | mL/min |
| | operated at 35° C. | 3% B, 2.5 min 98% | | |
| | A: 0.05% aq. HCO₂H | B, 3.5 min 98%, 3.6 | | |
| | B: 0.05% HCO₂H in ACN | min 3% B, 4 min 3% | | |
| | | B | | |
| 3 | Acquity BEH C18 50 × 2.1 mm 1.7 μm | 0 min 3% B, 2.5 min | 0.6 | mL/min |
| | operated at 35° C. | 3% B, 7.5 min 98% | | |
| | A: 0.05% aq. HCO₂H | B, 9.6 min 3% B, 10 | | |
| | B: 0.05% HCO₂H in ACN | min 3% B | | |
| 4 | Acquity BEH C18 100 × 2.1 mm 1.7 | 0 min 3% B, 8.5 min | 0.55 | mL/min |
| | μm operated at 50° C. | 100% B, 9 min 100% | | |
| | A: 0.05% aq. HCO₂H | B, 9.5 min 3% B, 10 | | |
| | B: 0.05% HCO₂H in ACN | min 3% B | | |
| 5 | Acquity BEH C18 50 × 2.1 mm 1.7 μm | 0 min 3% B, 0.4 min | 0.6 | mL/min |
| | operated at 35° C. | 3% B, 2.5 min 98% | | |
| | A: 0.05% aq. TFA | B, 3.5 min 98% B, | | |
| | B: 0.05% TFA in ACN | 3.6 min 3% B, 4 min | | |
| | | 3% B | | |
| 6 | Acquity BEH C18 50 × 2.1 mm 1.7 μm | 0 min 3% B, 2.5 min | 0.6 | mL/min |
| | operated at 35° C. | 3% B, 7.5 min 98% | | |
| | A: 0.05% aq. TFA | B, 9.5 min 98% B, | | |
| | B: 0.05% TFA in ACN | 9.6 min 3% B, 10 min | | |
| | | 3% B | | |
| 7 | Acquity BEH C18 100 × 2.1 mm 1.7 | 0 min 3% B, 16 min | 0.45 | mL/min |
| | μm operated at 50° C. | 100% B, 18 min | | |
| | A: 0.05% aq. TFA | 100% B, 18.5 min 3% | | |
| | B: 0.05% TFA in ACN | B, 20 min 3% B | | |
| 8 | Xbridge C18 75 × 4.6 mm 3.5 μm | 0 min 5% B, 0.5 min | 1.3 | mL/min |
| | operated at 35° C. | 5% B, 1 min 15% B, | | |
| | A: 10 mM aq. NH₄HCO₃ | 4 min 98% B, 7 min | | |
| | B: ACN | 98% B, 7.5 min 5% | | |
| | | B, 8 min 5% B | | |
| 9 | X Bridge C18 150 × 4.6 mm 3.5 μm | 0 min 5% B, 1 min | 1.0 | mL/min |
| | operated at 35° C. | 5% B, 3 min 15% B, | | |
| | A: 10 mM aq. NH₄HCO₃ | 7 min 55% B, 11 min | | |
| | B: ACN | 98% B, 16 min 98% | | |
| | | B, 16.1 min 5% B, 20 | | |
| | | min 5% B | | |
| 10 | X SELECT C18 150 × 4.6 mm 3.5 μm | 0 min 5% B, 1 min | 1.0 | mL/min |
| | operated at RT | 5% B, 3 min 15% B, | | |
| | A: 10 mM aq. NH₄HCO₃ | 7 min 55% B, 11 min | | |
| | B: ACN | 98% B, 16 min 98% | | |
| | | B, 16.1 min 5% B, 20 | | |
| | | min 5% B | | |
| 11 | Acquity UPLC HSS T3 50 × 2.1 mm | 0 min 1% B, 0.5 min | 0.7 | mL/min |
| | 1.8 μm operated at 30° C. | 6% B, 1 min 6% B, | | |
| | A: 10 mM aq. NH₄OAc + 0.1% | 2.6 min 95% B, 3.8 | | |
| | HCO₂H | min 95% B, 3.81 min | | |
| | B: 0.1% HCO₂H in ACN | 1% B, 4.8 min 1% B | | |
| 12 | Acquity UPLC HSS T3 50 × 2.1 mm | 0 min 5% B, 0.9 min | 1.2-1.3 | mL/min |
| | 1.8 μm operated at 60° C. | 95% B, 1.2 min 95% | | |
| | A: 10 mM aq. NH₄OAc + 0.1% | B, 1.4 min 5% B | | |
| | HCO₂H | | | |
| | B: 0.1% HCO₂H in ACN | | | |
| 13 | X Brigde BEH C18 (3 × 100) mm, | 0 min 5% A, 5 min | 1 | mL/min |
| | 2.5 μm operated at 35° C. | 98% A, 9 min 98% A, | | |
| | A: 0.05% TFA in ACN | 9.01 min 5% A, 12 | | |
| | B: 0.05% aq. TFA | min 5% A | | |
| 14 | ACQUITY UPLC BEH C18 (2.1 × 100) | 0 min 10% A, 2.5 min | 0.4 | mL/min |
| | mm, 1.7 μm operated at 60° C. | 10% A, 7.5 min 98% | | |
| | A: 0.05% TFA in ACN | A, 9.5 min 98% A, | | |
| | B: 0.05% aq. TFA | 9.6 min 10% A, 10 | | |
| | | min 10% A | | |
| 15 | YMC-Triart C18 ExRS (75 × 4.6 mm, | 0 min 5% B, 0.8 min | 1.0 | mL/min |
| | 3 μm) operated at 45° C. | 5% B, 2 min 25% B, | | |
| | A: 10 mM aq. NH₄HCO₃ | 5 min 90% B, 7 min | | |
| | B: 100% ACN | 95% B, 8.5 min 95% | | |
| | | B, 8.6 min 5% B, 10 | | |
| | | min 5% B | | |
| 16 | X-BRIDGE C8 (4.6 × 75 mm) 3.5 μm | 0 min 5% B, 3 min | 1.0 | mL/min |
| | operated at 50° C. | 98% B, 5 min 98% B, | | |
| | A: 0.05% aq. HCO₂H | 5.5 min 5% B, 6 min | | |
| | B: 0.05% HCO₂H in ACN | 5% B | | |
| 17 | Xbridge C18 (75 × 4.6 mm, 3 μm) | 0 min 5% B, 0.5 min | 1.0 | mL/min |
| | operated at 50° C. | 5% B, 1 min 15% B, | | |
| | A: 10 mM aq. NH₄HCO₃ | 4 min 98% B, 7 min | | |
| | B: ACN | 98% B, 7.5 min 5% | | |
| | | B, 8 min 5% B | | |
| 18 | Acquity BEH C18 (50 mm × 2.1 mm, | 0 min 5% B, 5 min | 0.6 | mL/min |
| | 1.7 μm) operated at 35° C. | 98% B, 9 min 98% B, | | |
| | A: 0.05% aq. HCO₂H | 9.01 min 5% D, 12 | | |
| | B: 0.05% TFA in ACN | min 5% B | | |
| 19 | ACQUITY UPLC BEH C18 (2.1 × 100) | 0 min 10% A, 2.5 min | 0.4 | mL/min |
| | mm, 1.7 μm operated at 45° C. | 10% A, 7.5 min 98% | | |
| | A: 0.05% TFA in ACN | A, 9.5 min 98% A, | | |
| | B: 0.05% aq. TFA | 9.6 min 10% A, 10 | | |
| | | min 10% A | | |
| 20 | ACQUITY UPLC BEH C18 (2.1 × 100) | 0 min 10% A, 2.5 min | 0.4 | mL/min |
| | mm, 1.7 μm operated at 35° C. | 10% A, 7.5 min 98% | | |
| | A: 0.05% TFA in ACN | A, 9.5 min 98% A, | | |
| | B: 0.05% aq. TFA | 9.6 min 10% A, 10 | | |
| | | min 10% A | | |
| 21 | ACQUITY UPLC BEH C18 (2.1 × 100) | 0 min 10% A, 2.5 min | 0.55 | mL/min |
| | mm, 1.7 μm operated at 50° C. | 10% A, 7.5 min 98% | | |
| | A: 0.05% TFA in ACN | A, 9.5 min 98% A, | | |
| | B: 0.05% aq. TFA | 9.6 min 10% A, 10 | | |
| | | min 10% A | | |
| 22 | ACQUITY UPLC BEH C18 (2.1 × 100) | 0 min 10% A, 2.5 min | 0.65 | mL/min |
| | mm, 1.7 μm operated at 35° C. | 10% A, 7.5 min 98% | | |
| | A: 0.05% TFA in ACN | A, 9.5 min 98% A, | | |
| | B: 0.05% aq. TFA | 9.6 min 10% A, 10 | | |
| | | min 10% A | | |
| 23 | X Bridge C18 (50 mm × 4.6 mm, 3.5 μm) | 0 min 5% B, 0.5 min | 0.8 | mL/min |
| | operated at 45° C. | 5% B, 5 min 98% B, | | |
| | A: 10 mM aq. NH₄HCO₃ | 7.5 min 98% B, 7.6 | | |
| | B: ACN | min 5% B, 9 min 5% | | |
| | | B | | |
| 24 | YMC-Triart C18 ExRS (50 × 2.1 mm, | 0 min 5% B, 5 min | 0.6 | mL/min |
| | 1.9 μm) operated at 35° C. | 98% B, 9 min 98% B, | | |
| | A: 0.05% aq. TFA | 9.01 min 5% B, 12 | | |
| | B: 0.05% TFA in ACN | min 5% B | | |
| 25 | X Bridge C18 50 × 4.6 mm 3.5 μm | 0 min 2% B, 0.5 min | 0.8 | mL/min |
| | operated at 35° C. | 2% B, 3 min 98% B, | | |
| | A: 10 mM aq. NH₄HCO₃ | 6 min 98% B, 8 min | | |
| | B: ACN | 2% B | | |
| 26 | X Bridge C18 50 × 4.6 mm 3.5 μm | 0 min 5% B, 0.5 min | 1.3 | mL/min |
| | operated at 30° C. | 5% B, 1.0 min 15% | | |
| | A: 10 mM aq. NH₄HCO₃ | B, 4.0 min 98% B, | | |
| | B: ACN | 7.0 min 98% B, 7.5 | | |
| | | min 5% B, 8.0 min | | |
| | | 5% B | | |
| 27 | X Bridge C18 50 × 4.6 mm 3.5 μm | 0 min 2% B, 0.5 min | 1.0 | mL/min |
| | operated at 45° C. | 2% B, 2.5 min 98% | | |
| | A: 10 mM aq. NH₄HCO₃ | B, 5 min 98% B, 5.1 | | |
| | B: ACN | min 2% B, 6 min 2% | | |
| | | B | | |
| 28 | YMC Triart C18 (75 × 4.6 mm, 3 um) | 0 min 5% B, 0.5 min | 1.3 | mL/min |
| | operated at 45° C. | 5% B, 1 min 15% B, | | |
| | A: 5 mM aq. NH₄HCO₃ | 6 min 55% B, 9 min | | |
| | B: ACN | 95% B, 12 min 95% | | |
| | | B, 13 min 5% B, 14 | | |
| | | min 5% B | | |
| 29 | YMC Triart C18 (75 × 2.1 mm, 1.9 um) | 0 min 3% B, 0.4 min | 0.6 | mL/min |
| | operated at 35° C. | 3% B, 2.5 min 98% | | |
| | A: 0.05% aq. TFA | B, 3.5 min 98% B, | | |
| | B: 0.05% TFA in ACN | 3.6 min 3% B, 4 min | | |
| | | 3% B | | |
| 30 | YMC Triart C18 (50 × 2.1 mm, 1.9 um) | 0 min 3% A, 2.5 min | 0.6 | mL/min |
| | operated at 60° C. | 3% A, 7.5 min 98% | | |
| | A: 0.05% aq. TFA | A, 9.5 min 98% A, | | |
| | B: 0.05% TFA in ACN | 9.6 min 3% A, 10 min | | |
| | | 3% A | | |
| 31 | YMC Triart C18 (50 × 2.1 mm, 1.9 um) | 0 min 3% A, 0.4 min | 0.6 | mL/min |
| | operated at 60° C. | 3% A, 2.5 min 98% | | |
| | A: 0.05% aq. TFA | A, 3.5 min 98% A, | | |
| | B: 0.05% TFA in ACN | 3.6 min 3% A, 4 min | | |
| | | 3% A | | |
| 32 | YMC Triart C18 (50 × 4.6 mm, 1.9 um) | 0 min 5% A, 0.5 min | 1.0 | mL/min |
| | operated at 60° C. | 5% A, 3 min 95% A, | | |
| | A: 0.05% TFA in ACN | 6 min 95% A, 6.1 min | | |
| | B: 0.05% aq. TFA | 5% A, 8 min 5% A | | |
| 33 | X Bridge C18 75 × 4.6 mm 3.5 μm | 0 min 5% B, 0.5 min | 1.0 | mL/min |
| | operated at 45° C. | 5% B, 1 min 15% B, | | |
| | A: 5 mM aq. NH₄HCO³ | 6 min 55% B, 9 min | | |
| | B: ACN | 95% B, 12 min 95% | | |
| | | B, 13 min 5% B, 14 | | |
| | | min 5% B | | |
| 34 | X Bridge C18 75 × 4.6 mm 3.5 μm | 0 min 5% B, 0.5 min | 1.3 | mL/min |
| | operated at 35° C. | 5% B, 1.0 min 15% | | |
| | A: 10 mM aq. NH₄HCO₃ | B, 4.0 min 98% B, | | |
| | B: ACN | 7.0 min 98% B, 7.5 | | |
| | | min 5% B, 8.0 min | | |
| | | 5% B | | |
| 35 | X Brigde BEH C18 (3 × 100) mm, | 0 min 3% A, 4 min | 1 | mL/min |
| | 2.5 μm operated at 35° C. | 98% A, 5 min 98% A, | | |
| | A: 0.05% TFA in ACN | 5.01 min 3% A, 6 min | | |
| | B: 0.05% aq. TFA | 3% A | | |
| 36 | X Bridge C18 (100 mm × 3 mm, 2.5 μm) | 0 min 3% A, 4 min | 1.0 | mL/min |
| | operated at 50° C. | 98% A, 5 min 98% A, | | |
| | A: 0.05% aq. TFA | 5.01 min 3% A, 6 min | | |
| | B: 0.05% TFA in ACN | 3% A | | |
| 37 | X Bridge C18 (75 mm × 4.6 mm, 3.5 μm) | 0 min 5% B, 0.8 min | 1.0 | mL/min |
| | operated at 45° C. | 5% B, 2 min 25% B, | | |
| | A: 10 mM aq. NH₄HCO₃ | 5 min 90% B, 7 min | | |
| | B: 100% ACN | 95% B, 8.6 min 5% | | |
| | | B, 10 min 5% B | | |
| 38 | Acquity BEH C18 100 × 2.1 mm 1.7 | 0 min 3% A, 1 min | 0.55 | mL/min |
| | μm operated at 60° C. | 10% A, 8.5 min 100% | | |
| | A: 0.05% TFA in ACN | A, 9 min 100% A, 9.5 | | |
| | B: 0.05% aq. TFA | min 3% A, 10 min | | |
| | | 3% A | | |
| 39 | Acquity BEH C18 100 × 2.1 mm 1.7 | 0 min 3% A, 8 min | 0.45 | mL/min |
| | μm operated at 60° C. | 100% A, 9 min 100% | | |
| | A: 0.05% aq. TFA | A, 9.5 min 3% A, 10 | | |
| | B: 0.05% TFA in ACN | min 3% A | | |
| 40 | Acquity BEH C18 100 × 2.1 mm 1.7 | 0 min 10% B, 2.5 min | 0.4 | mL/min |
| | μm operated at 45° C. | 10% B, 7.5 min 98% | | |
| | A: 0.05% aq. TFA | B, 9.5 min 98% B, | | |
| | B: 0.05% TFA in ACN | 9.6 min 10% B, 10 | | |
| | | min 10% B | | |
| 41 | Agilent Poroshell 120 SB-C18 | 0 min 1% B, 1.5 min | 3 | mL/min |
| | 4.6 × 30 mm 2.7 μm operated at 45° C. | 100% B, 2.2 min | | |
| | A: 0.1% aq HCO₂H | 100% B | | |
| | B 0.1% HCO₂H in ACN | | | |
| 42 | Waters HSS T3 1.8 μm, 1.0 × 50 mm | 0 min 10% B, 0.1 min | 0.475 | mL/min |
| | column operated at 50° C. | 10% B, 0.6 min 95% | | |
| | A: 0.01% aq HCO₂H | B, 1.5 min 95% B, | | |
| | B 0.01% HCO₂H in AC | 1.51 min 10% B | | |

Abbreviations

ABPR = automated back pressure regulator
ACN = acetonitrile
AcOH = acetic acid
AdBrettPhos Pd G3 = [2-(di-1-adamantyl-phosphino)-2′,4′,6′-tri-iso-
propyl-3,6-dimethoxybiphenyl][2-(2′-amino-1,1′-biphenyl)]palladium(II)
methanesulfonate
aq = aqueous
Boc = tert-butoxycarbonyl
Boc₂O = di-tert-butyl dicarbonate
BOP = (benzotriazol-1-yloxy)tris(dimethylamino)-phosphonium
hexafluorophosphate
B₂Pin₂ = 4,4,4′,4′,5,5,5′,5′-octamethyl-2,2′-bi(1,3,2-dioxaborolane)
Brettphos Pd G3 = [(2-Di-cyclo-hexylphosphino-3,6-dimethoxy-2′,4′,6′-tri-
iso-propyl-1,1′-biphenyl)-2-(2′-amino-1,1′-biphenyl)]palladium(II)
methanesulfonate methanesulfonate
BrettPhos Pd G4 = [2′-(methylamino)-[1,1′-biphenyl]-2-yl]palladio
methanesulfonate di-cyclo-hexyl[3,6-dimethoxy-2′,4′,6′-tris(propan-2-yl)-
[1,1′-biphenyl]-2-yl]phosphane
brine = saturated aqueous sodium chloride
cataCXium Pd G4 = [di(adamantan-1-
yl)(butyl)phosphine](methanesulfonato-κO)[2′-(methylamino)-2-
biphenylyl]palladium
CBz = benzyloxycarbonyl
CBz-Cl = benzyl chloroformate
CDI = 1,1′-carbonyldiimidazole
DAST = diethylaminosulfur trifluoride
dba = dibenzylideneacetone
DCC = di-cyclo-hexylcarbodiimide
DCE = 1,2-dichloroethane
DCM = dichloromethane
DEA = diethylamine
DEAD = diethyl azodicarboxylate
Deoxo-Fluor = bis(2-methoxyethyl)aminosulfur trifluoride
DIAD = di-iso-propyl azodicarboxylate
DIPEA = di-iso-propyl ethyl amine
DMAP = 4-(dimethylamino)pyridine
DME = 1,2-dimethoxyethane
DMF = dimethyl formamide
DMDCH = trans-N,N′-dimethyl-cyclo-hexane-1,2-diamine
DMEDA = N1,N2-dimethylethane-1,2-diamine
DMP = Dess-Martin periodinane
DMS = dimethyl sulfide
DMSO = dimethyl sulfoxide
DPPF = 1,1′-bis(diphenylphosphino)ferrocene
EDC = N-(3-dimethylaminopropyl)-N′-ethylcarbodi-imide
EtOAc = ethyl acetate
EtOH = ethanol
FC = flash chromatography on silica gel unless stated otherwise from the
eluent described in the brackets
h = hour(s)
HATU = (1-[bis(dimethylamino)methylene]-1H-1,2,3-triazolo[4,5-
b]pyridinium 3-oxide hexafluorophosphate
HBTU = N,N,N′,N′-tetramethyl-O-(1H-benzotriazol-1-yl)uronium
hexafluorophosphate
HFIP = 1,1,1,3,3,3-hexafluoro-2-propanol
HOBt = hydroxybenzotriazole
HPLC = high performance liquid chromatography
Int./Ints. = intermediate/intermediates
IPA = iso-propyl alcohol
KOAc = poassium acetate
KOtBu = potassium tert-butoxide
LCMS = liquid chromatography-mass spectrometry
LDA = lithium di-iso-propylamide
LiHMDS = lithium bis(trimethylsilyl)amide
mCPBA = m-chloro-perbenzoic acid
MeOH = methanol
2MeTHF = 2-methyl-tetrahydrofuran
MHz = megahertz
MS = molecular sieves
MsCl = methanesulfonyl chloride (mesyl chloride)
MTBE = methyl tert-butyl ether
MW = microwave
NaOtBu = sodium tert-butoxide
NBS = N-bromosuccinimide
NMP = N-methyl-2-pyrrolidon
NMR = nuclear magnetic resonance
ON = overnight
Pd₂dba₃ = tris(dibenzylideneacetone)dipalladium(0)
PdDPPFCl₂-DCM = [1,1′-bis(diphenylphosphino)-
ferrocene]dichloropalladium(II) DCM complex
Pd G3 SPhos = (2-Di-cyclo-hexylphosphino-2′,6′-dimethoxybiphenyl) [2-
(2′-amino-1,1′-biphenyl)]palladium(II) methanesulfonate
Pd G3 tert-butyl-Xphos = methanesulfonato (di-tert-butyl)
phenylphosphino (2′-amino-1,1′-biphenyl-2-yl) palladium(II)
ppm = parts per million
PyBOP = (benzotriazol-1-yloxy)tripyrrolidino-phosphonium
hexafluorophosphate
RT = room temperature
RU = response units
RuPhos = 2-di-cyclo-hexylphosphino-2′,6′-di-iso-propoxybiphenyl
Ruphos Pd-G2 = Chloro(2-dicyclohexylphosphino-2′,6′-diisopropoxy-1,1′-
biphenyl)[2-(2′-amino-1,1′-biphenyl)]palladium(II)
sat. = saturated
SCX = strong cation exchange
SEM = 2-(trimethylsilyl)ethoxymethyl
SFC = supercritical fluid chromatography
SPR = Surface plasmon resonance
STAB = sodium triacetoxy borohydride
TBAF = tetra n-butyl ammonium fluoride
TBAI = tetra n-butyl ammonium iodide
TFA = trifluoro acetic acid
Tf₂O = trifluoromethanesulfonic anhydride
THF = tetrahydrofuran
TLC = thin layer chromatography
TMEDA = tetramethylethylenediamine
Ts = tosyl
TsOH = p-toluenesulfonic acid
T3P = propanephosphonic acid anhydride
VCD = Vibrational circular dichroism
XantPhos = 4,5-bis(diphenylphosphino)-9,9-dimethylxanthene
XPhos = 2-di-cyclo-hexylphosphino-2′,4′,6′-tri-iso-propylbiphenyl

### General Synthesis Methods

The compounds of the present disclosure can be prepared in a number of ways well known to those skilled in the art of synthesis. The compounds of the disclosure could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present disclosure or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 6OF254 silica-gel TLC plates. Visualisation of TLC plates was performed using UV light (254 nm) or by an appropriate staining technique.

The compounds of the disclosure can be prepared according to the bond formations as outlined below.

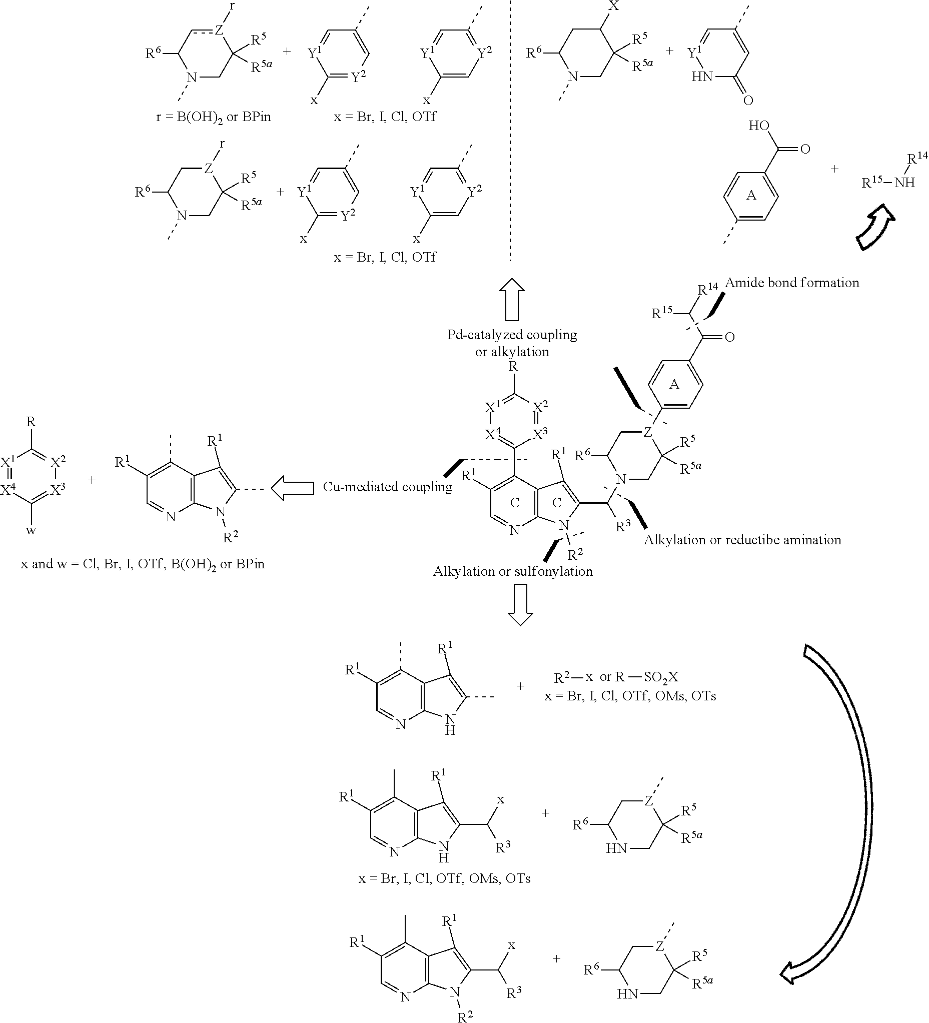

Formation of the amide bond linking (R¹⁵)(R¹⁴)N to the rest of the structure can be performed from the corresponding acid, the (R¹⁵)(R¹⁴)NH amine, and a suitable coupling agent for example under the conditions used to prepare Int. 1AF84.

Linking the A and B rings can be achieved in palladium-mediated couplings reactions wherein either an amine (Z═N) is reacted with an aryl (pseudo)halide based on ring A or by coupling ring B in which Z is a carbon atom linked to a boronic acid or boronate to aryl (pseudo)halide based on ring A. Such reactions can be performed as described for the syntheses of Ints. 1M51 and 1S3. Compounds in which Ring A is a pyridone can be prepared from precursors like Int. 1AE1.

The bond connecting the B ring to the azaindole core (C—C) can be formed either by alkylation of the B ring amine or lactam with an alkyl (pseudo)halide liked to the azaindole core (C—C) or by reductive amination of the B ring amine to the ketone/aldehyde liked to the azaindole core (C—C). Such reactions can be performed as described for the synthesis of Ints. 1AB2/1AB3, Example 1n7 from Int. 1O4, and Example 1q2 from Int. 1Q1.

R² can be attached to the parent azaindole core (C—C) by alkylation with a suitable alkylation agent. Such reactions can be performed as described for the syntheses of Ints. 1AB8 and 1K3. Compounds wherein R¹ is not hydrogen can be prepared from precursors like Int. 1H2 or 1Y3.

Attaching ring D to the azaindole core (C—C) can be done either by copper-mediated coupling of a boronic acid or boronate linked to the azaindole (C—C) core and the heteroaromatic ring D bearing a (pseudo)halogen or by the inverted set of Suzuki reaction partners. Such reactions can be performed as described for the synthesis of Example 1af64 from Int. 1AF75 or Example 1af52 from 1AF76′.

### Preparation of Intermediates

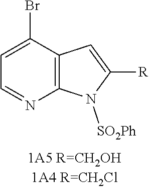

4-Bromo-1-(phenyl-sulfonyl)-1H-pyrrolo[2,3-b]pyridine-2-carbaldehyde (0.27 g) and NaBH₄ (33 mg) were stirred 0.5 h in THF/MeOH (9:1, 5 mL) at 0° C. The OL (water/EtOAc) was washed with brine, dried, and concentrated to give Int. 1A5 (4-bromo-1-(phenylsulfonyl)-1H-pyrrolo[2,3-b]pyridin-2-yl)methanol (281 mg). Int. 1A5 (0.24 g) and MsCl (0.052 mL) was stirred 0.5 h in DCM/Et₃N (29:1, 5.2 mL). MsCl (0.014 mL) was added and stirring continued 0.5 h. The OL (water/DCM) was washed with brine, dried, concentrated, and purified by FC (heptane/EtOAc 1:0 to 1:1) to give Int. 1A4 4-bromo-2-(chloromethyl)-1-(phenylsulfonyl)-1H-pyrrolo[2,3-b]pyridine (0.15 g).

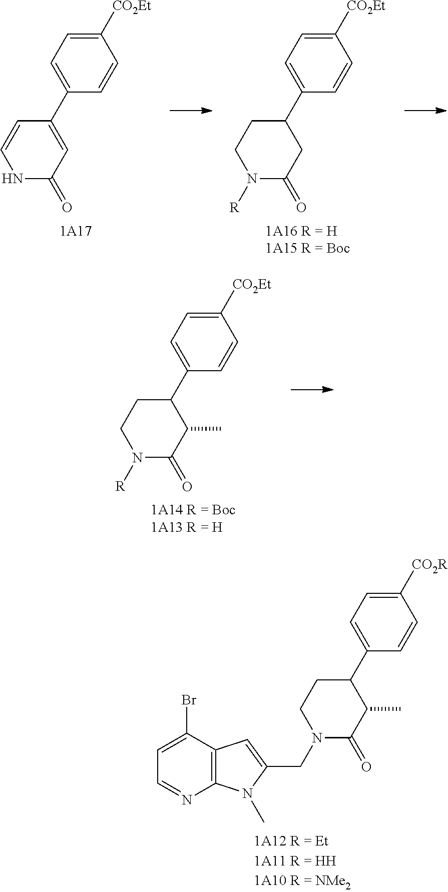

4-Bromopyridin-2(1H)-one (1.0 g), ethyl 4-(4,4,5,5-tetramethyl-1,3,2-dioxaborolan-2-yl)benzoate (3.17 g), PdDPPFCl₂-DCM (0.42 g), and K₃PO₄ (3.66 g) were degassed in dioxane/water (10:1, 11 mL) and stirred 2 h at 110° C. under MW conditions. The mixture was filtered, concentrated, and purified by FC (DCM/MeOH 95:5) to give Int. 1A17 ethyl 4-(2-oxo-1H-pyridin-4-yl)benzoate (0.50 g). Int. 1A17 (3.5 g) was hydrogenated 48 h using 10% Pd/C (1.5 g) in EtOH/AcOH (4:1, 50 mL), filtered, concentrated, and purified by FC (hexane/EtOAc 7:3) to give Int. 1A16 ethyl 4-(2-oxopiperidin-4-yl)-benzoate (2.5 g). Int. 1A16 (0.50 g), Boc₂O (0.61 g), and DMAP (23 mg) were stirred ON in DCM/Et₃N (25:1, 21 mL) at 0° C. to RT ON. The OL (EtOAc/water) was washed with brine, dried, concentrated, and purified by FC (hexane/EtOAc 7:3) to give Int. 1A15 tert-butyl 4-(4-ethoxy-carbonylphenyl)-2-oxo-piperidine-1-carboxylate (0.40 g). Int. 1A15 (0.30 g) was stirred 0.5 h in 0.1M LiHMDS in THF (11 mL) at −78° C. CH₃I (0.16 mL) was added and stirring continued 2 h at −25 to −30° C. The OL (sat. aq. NH₄Cl/EtOAc) was washed with water and brine, dried, concentrated, and purified by FC (hexane/EtOAc 9:1) to give Int. 1A14 tert-butyl trans-4-(4-ethoxycarbonylphenyl)-3-methyl-2-oxo-piperidine-1-carboxylate (0.18 g). Int. 1A14 (0.60 g) was stirred ON in DCM/TFA (32:1, 20.6 mL) at 0° C. to RT and concentrated. The OL (10% MeOH in DCM/sat. aq. NaHCO₃) was dried, and concentrated to give Int. 1A13 ethyl 4-[trans-3-methyl-2-oxo-4-piperidyl]benzoate (0.38 g). Int. 1A13 (0.20 g) and NaH (28 mg) were stirred 0.5 h in THF (10 mL) at 0° C. to RT. Int. 1J1 (0.22 g) and TBAI (25 mg) were added and stirring continued ON. The OL (aq. citric acid/EtOAc) was dried and concentrated to give Ints. 1A12/1A11 ethyl 4-[trans-1-[(4-bromo-1-methyl-pyrrolo[2,3-b]pyridin-2-yl)methyl]-3-methyl-2-oxo-4-piperidyl]benzoate and 4-[trans-1-[(4-bromo-1-methyl-pyrrolo[2,3-b]pyridin-2-yl)methyl]-3-methyl-2-oxo-4-piperidyl]benzoic acid (0.30 g). 20 mg of this mixture, HNMe₂ (0.11 g, HCl salt), and HATU (0.25 g) were stirred ON in DMF/DIPEA (28.6:1, 10.4 mL). The mixture was combined with another batch prepared similarly on 30 mg scale, concentrated, and purified by FC (hexane/EtOAc 1:4) to give Int. 1A10 N,N-Dimethyl-4-[trans-1-[(4-bromo-1-methyl-pyrrolo[2,3-b]pyridin-2-yl)methyl]-3-methyl-2-oxo-4-piperidyl]benzamide (0.20 g).

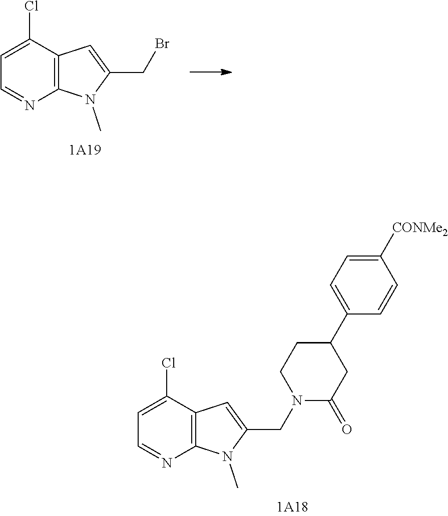

Int. 1A18 4-[1-[(4-Chloro-1-methyl-pyrrolo[2,3-b]pyridin-2-yl)methyl]-2-oxo-4-piperidyl]-N,N-dimethyl-benzamide was prepared similarly to Int. 1A10 from Int. 1A16 and 4-chloro-2-(chloromethyl)-1-methyl-1H-pyrrolo[2,3-b]pyridine (prepared similarly to Int. 1J1).

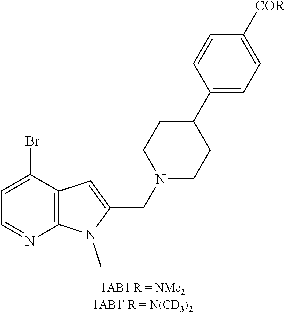

Int. 1J1 (8 g) and N,N-dimethyl-4-(4-piperidyl)benzamide (9.9 g, HCl salt) were stirred ON in DMF/DIPEA (0.7:1, 47 mL) at 0° C. to RT. The OL (water/EtOAc) was washed with brine, dried, concentrated, and purified by FC (hexane/EtOAc 1:3) to give Int. 1AB1 4-(1-((4-Bromo-1-methyl-1H-pyrrolo[2,3-b]pyridin-2-yl)methyl)piperidin-4-yl)-N,N-dimethylbenzamide (9 g). Int. 1AB1′ 4-(1-((4-bromo-1-methyl-1H-pyrrolo[2,3-b]pyridin-2-yl)methyl)piperidin-4-yl)-N,N-bis(methyl-d₃)benzamide was prepared similarly from HN(CD₃)₂.

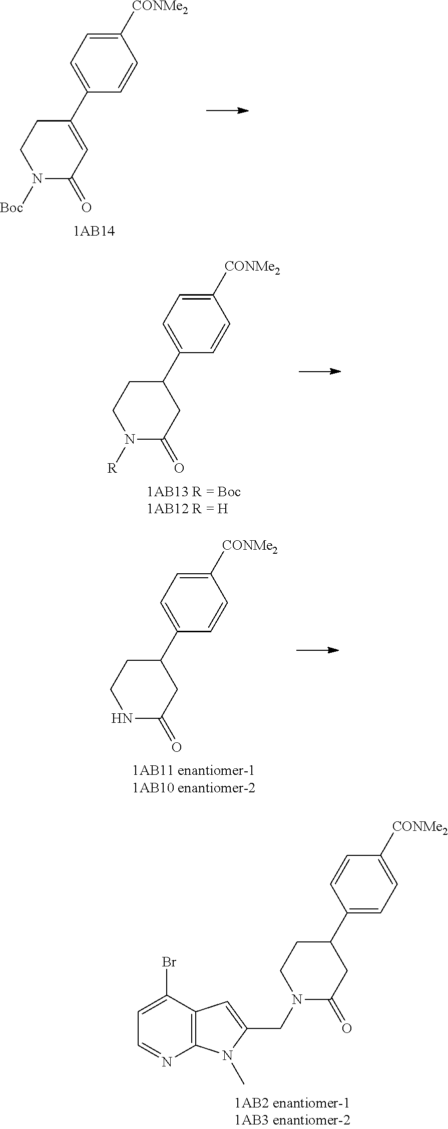

Na₂CO₃ (9.2 g), 1,1,1-trifluoro-N-phenyl-N-((trifluoromethyl)sulfonyl)methane-sulfonamide (10 g), and (4-(dimethylcarbamoyl)phenyl)boronic acid (5.5 g) were degassed in toluene/water (4:1, 250 mL) and stirred 3 h at 100° C. The mixture was filtered, concentrated, and purified by FC (hexane/EtOAc 2:3) to give Int. 1AB14 tert-butyl 4-[4-(dimethylcarbamoyl)phenyl]-6-oxo-2,3-dihydropyridine-1-carboxylate (5.1 g). Int. 1AB14 (4.4 g) was hydrogenated ON using 10% Pd/C (0.45 g) in MeOH (100 mL), filtered, and concentrated to give Int. 1AB13 tert-butyl 4-(4-(dimethylcarbamoyl)-phenyl)-2-oxopiperidine-1-carboxylate (3.9 g). Int. 1AB13 (8.1 g) was stirred ON in DCM/4M HCl in dioxane (4.2:1, 124 mL) and concentrated. The OL (sat. aq. NaHCO₃/10% MeOH in DCM) was dried and concentrated to give Int. 1AB12 N,N-dimethyl-4-(2-oxo-piperidin-4-yl)-benzamide (2.9 g). This material was resolved by SFC using a Chiralpak IG 250×25 5 μm column operated at 30° C. and an eluent of 50% CO₂ and 50% MeOH (100 g/min) and a back pressure of 100 bar to give Int. 1AB11 (0.93 g, first peak) and Int. 1AB12 (0.91 g, second peak). The absolute configurations of these compounds were not determined. Int. 1AB12 (95 mg) and NaH (20 mg) were stirred 0.25 h in DMF (1 mL). Int. 1J1 (0.10 g) was added and stirring continued 0.5 h. The OL (DCM/water) was washed with water and brine, dried, concentrated, and triturated in ACN to give Int. 1AB3 (S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (31 mg). Int. 1AB2 (S)-4-(1-((4-Bromo-1-methyl-1H-pyrrolo[2,3-b]pyridin-2-yl)methyl)-2-oxopiperidin-4-yl)-N,N-dimethylbenzamide or (R)-4-(1-((4-bromo-1-methyl-1H-pyrrolo[2,3-b]pyridin-2-yl)methyl)-2-oxopiperidin-4-yl)-N,N-dimethyl-benzamide (35 mg) was prepared similarly from Ints. 1AB11/1J1 (95 mg/0.10 g). The absolute configurations of these compounds were not determined.

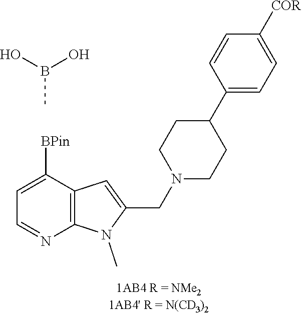

Int. 1AB1 (0.15 g), B₂Pin₂ (0.13 g), KOAc (81 mg), and PdDPPFCl₂-DCM (27 mg) were degassed in dioxane (3 mL) and stirred at 90° C., filtered and concentrated to give Ints. 1AB4/1AB4′ N,N-Dimethyl-4-[1-[[1-methyl-4-(4,4,5,5-tetramethyl-1,3,2-dioxaborolan-2-yl)pyrrolo[2,3-b]pyridin-2-yl]methyl]-4-piperidyl]benzamide and [2-[[4-[4-(dimethylcarbamoyl)phenyl]-1-piperidyl]methyl]-1-methyl-pyrrolo[2,3-b]pyridin-4-yl]boronic acid and N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)piperidin-4-yl)benzamide and (2-((4-(4-(bis-(methyl-d₃)carbamoyl)phenyl)piperidin-1-yl)methyl)-1-methyl-1H-pyrrolo[2,3-b]pyridin-4-yl)boronic acid.

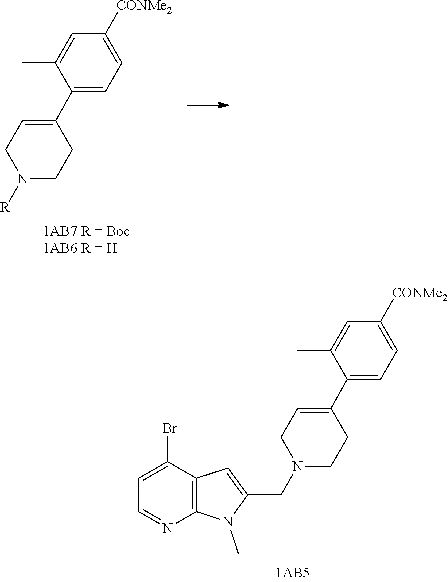

4-Bromo-N,N,3-trimethylbenzamide (9.0 g), tert-butyl 4-(4,4,5,5-tetramethyl-1,3,2-dioxaborolan-2-yl)-3,6-dihydro-2H-pyridine-1-carboxylate (15.2 g), PdDPPFCl₂-DCM (0.67 g), and Na₂CO₃ (9.62 g) were degassed in dioxane (180 mL) and stirred ON at 90° C. The mixture was filtered, concentrated, and purified by FC (hexane/EtOAc 2:3) to give Int. 1AB7 tert-butyl 4-[4-(dimethylcarbamoyl)-2-methyl-phenyl]-3,6-dihydro-2H-pyridine-1-carboxylate (11.0 g). Int. 1AB7 (0.6 g) was stirred ON in DCM/TFA (30:1, 15.5 mL), concentrated, and triturated in Et₂O to give Int. 1AB6 N,N,3-trimethyl-4-(1,2,3,6-tetrahydropyridin-4-yl)benzamide (TFA salt). Ints. 1AB6/1AB8 (0.23 g, TFA salt/0.1 g) were stirred ON in DCE/DIPEA (11.4:1, 5.4 mL) at 0° C. to RT. STAB (26 mg) was added and stirring continued 2 h at 0° C. to RT. The OL (water/DCM) was dried, concentrated, and purified by FC (MeOH/DCM 1:9) to give Int. 1AB5 4-(1-((4-Bromo-1-methyl-1H-pyrrolo[2,3-b]pyridin-2-yl)methyl)-1,2,3,6-tetrahydropyridin-4-yl)-N,N,3-trimethylbenzamide (0.10 g).

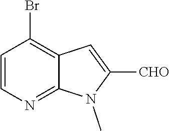

4-Bromo-1-methyl-1H-pyrrolo[2,3-b]pyridine (5.0 g) was stirred 2 h in 1.1M LDA in THF (33 mL) at −78° C. DMF (3.46 g in THF (20 mL)) was added and stirring continued ON at −78° C. to RT ON. The OL (sat. aq. NH₄Cl/EtOAc) was washed with water, brine, dried, concentrated, and triturated in EtOAc/pentane to give Int. 1AB8 4-Bromo-1-methyl-1H-pyrrolo[2,3-b]pyridine-2-carbaldehyde (3.1 g).

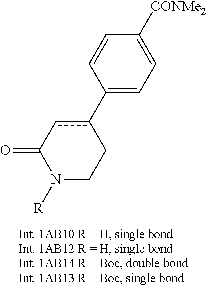

PdDPPFCl₂-DCM (1.1 g), tert-Butyl 6-oxo-4-(((trifluoromethyl)sulfonyl)oxy)-3,6-dihydropyridine-1(2H)-carboxylate (10.2 g), (4-(dimethylcarbamoyl)phenyl)boronic acid (5.7 g), and Na₂CO₃ (9.4 g) were stirred 3 h in toluene/water (150/40 mL) at 100° C. The mixture was filtered. The filtrate was concentrated and purified by FC (EtOAc/Hexane 3:2) to give Int. 1AB14 tert-butyl 4-(4-(dimethyl-carbamoyl)-phenyl)-6-oxo-3,6-dihydropyridine-1(2H)-carboxylate (5.2 g). Int. 1AB14 (5 g) and 10% Pd/C (2.5 g) were hydrogenated 48 h under a hydrogen pressure of 60 psi in EtOAc (100 mL). The mixture was filtered and concentration to give Int. 1AB13 tert-butyl 4-(4-(dimethylcarbamoyl)phenyl)-2-oxopiperidine-1-carboxylate (4.0 g). Int. 1AB13 (0.1 g) was stirred 12 h in DCM/4M HCl in dioxane (5/0.4 mL). The residue after concentration was triturated in Et₂O to give Int. 1AB10/1AB12 N,N-dimethyl-4-(2-oxopiperidin-4-yl)benzamide (78 mg).

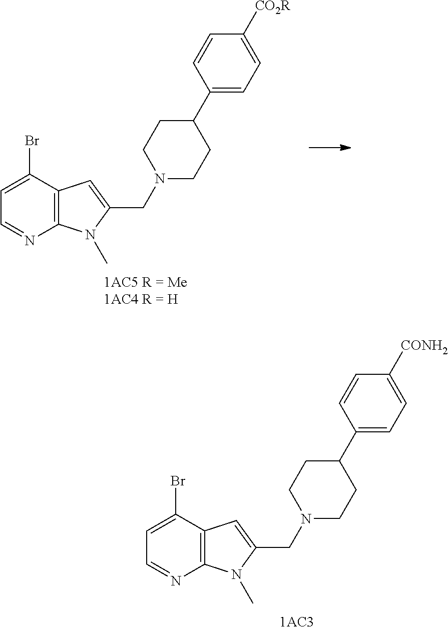

Int. 1AB8 (6.0 g) and methyl 4-(1-piperidin-4-yl)benzoate (7.1 g, HCl salt) were stirred 4 h in DCE/DIPEA (7.7:1, 113 mL). STAB (11.0 g) was added and stirring continued ON at 0° C. to RT. The OL (water/EtOAc) was washed with brine, dried, concentrated, and triturated in EtOAc/MeOH give Int. 1AC5 methyl 4-(1-((4-bromo-1-methyl-1H-pyrrolo[2,3-b]pyridin-2-yl)methyl)piperidin-4-yl)benzoate. Int. 1AC5 (0.4 g) and NaOH (0.18 g) were stirred ON in THF/MeOH (2:1, 9 mL), concentrated, and triturated in dilute aq. HCl to give Int. 1AC4 4-[1-[(4-bromo-1-methyl-pyrrolo[2,3-b]pyridin-2-yl)methyl]-4-piperidyl]-benzoic acid (0.38 g). Int. 1AC4 (0.5 g) and CDI (0.7 g) were stirred 2 h in DMF/Et₃N (30:1, 7.2 mL). 25% aq. NH₃ (1.51 mL) was added followed by water to precipitate a solid that was stirred ON in 1,1-dimethoxy-N,N-dimethylmethanamine (10 mL) to afford Int. 1AC3 4-(1-((4-bromo-1-methyl-1H-pyrrolo[2,3-b]pyridin-2-yl)methyl)piperidin-4-yl)benzamide (0.35 g).

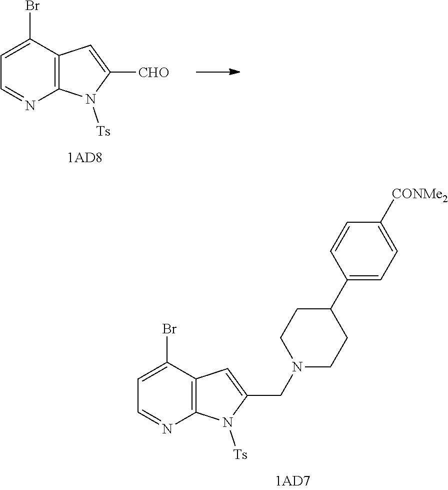

4-Bromo-1-tosyl-1H-pyrrolo[2,3-b]pyridine (1.0 g) was stirred 1 h in 0.6M LDA in THF (14.2 mL) at −78° C. DMF (1.3 mL) was added and stirring continued 2 h at −78° C. The OL (sat. aq. NH₄Cl/EtOAc) was dried, concentrated, and purified by FC (hexane/EtOAc 1:0 to 9:1) to give Int. 1AD8 4-bromo-1-(p-tolylsulfonyl)-pyrrol[2,3-b]-pyridine-2-carbaldehyde (0.6 g). N,N-Dimethyl-4-(4-piperidinyl)-benzamide (1.4 g, HCl salt) and Int. 1AD8 (1.3 g) were stirred ON in DCE/DIPEA (20:1, 21 mL) at 0° C. to RT. STAB (1.1 g) was stirring continued 2 h at 0° C. to RT. The OL (water/DCM) was dried, concentrated, and purified by FC (DCM/MeOH 9:1) to give Int. 1AD7 4-[1-[[4-bromo-1-(p-tolyl-sulfonyl)-pyrrolo[2,3-b]pyridin-2-yl]methyl]-4-piperidyl]-N,N-dimethyl-benzamide (2.3 g).

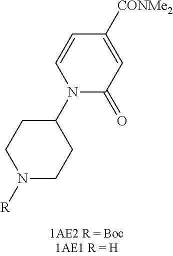

KOtBu (37 mg), tert-butyl 4-(p-tolylsulfonyloxy)piperidine-1-carboxylate (0.16 g), K₂CO₃ (83 mg), and N,N-dimethyl-2-oxo-1H-pyridine-4-carboxamide (50 mg) were degassed in DME (5 mL) and stirred ON at 120° C. The OL (water/MeOH/DCM) was dried and concentrated to give Int. 1AE2 tert-butyl 4-[4-(dimethyl-carbamoyl)-2-oxo-1-pyridyl]-piperidine-1-carboxylate. Int. 1AE2 (0.25 g) was stirred in DCM/TFA (8.6:1, 5.6 mL). The mixture was concentrated to give Int. 1AE1 N,N-Dimethyl-2-oxo-1-(piperidin-4-yl)-1,2-dihydropyridine-4-carboxamide (0.15 mg, TFA salt).

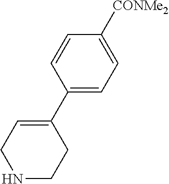

4-Bromo-N,N-dimethylbenzamide (25 g), tert-butyl 4-(4,4,5,5-tetramethyl-1,3,2-dioxaborolan-2-yl)-3,6-dihydropyridine-1(2H)-carboxylate (40.7 g), Na₂CO₃ (34.5 g), and PdDPPFCl₂-DCM (2.24 g) were degassed in dioxane/water (7.7:1, 260 mL) and stirred at 90° C. ON and filtered. The OL (water/EtOAc) was washed with brine, dried, concentrated, and purified by FC (heptane/EtOAc 55:45) to give tert-butyl 4-(4-(dimethyl-carbamoyl)-phenyl)-3,6-dihydropyridine-1(2H)-carboxylate (36.0 g). 5.0 g of this material was stirred ON in DCM/TFA (6.9:1, 46 mL) and concentrated. The OL (sat. aq. NaHCO₃/10% MeOH in DCM) was washed with brine, dried, concentrated, and triturated in Et₂O to give Int. 1AE7 4-(3,6-dihydro-2H-pyridin-4-yl)-N,N-dimethyl-benzamide (2.80 g).

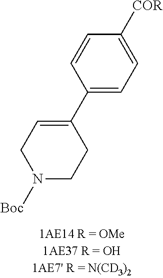

Methyl 4-bromobenzoic acid ester (10 g) tert-butyl 4-(4,4,5,5-tetramethyl-1,3,2-dioxaborolan-2-yl)-3,6-dihydropyridine-1(2H)-carboxylate (14.4 g), NaHCO₃ (11.7 g), and PdDPPFCl₂-DCM (1.2 g) were degassed in dioxane (300 mL) and stirred at 100° C. ON. The OL (water/EtOAc) was dried, concentrated, and purified by FC (hexane/EtOAc 9:1) to give Int. 1AE14 tert-butyl 4-(4-(methoxycarbonyl)phenyl)-3,6-dihydropyridine-1(2H)-carboxylate (11 g). 4.5 g of this material and LiOH—H₂O (3.0 g) were stirred 4 h in MeOH/THF/water (4:2:1, 35 mL) at 0° C. to RT. The mixture was partially concentrated and diluted with aq. citric acid to precipitate Int. 1AE37 4-(1-(tert-butoxycarbonyl)-1,2,3,6-tetrahydropyridin-4-yl)benzoic acid (3.9 g). Int. 1AE37 (4.0 g), HATU 7.52 g), and HN(CD₃)₂ (1.73 g, HCl salt) were stirred ON in DMF/DIPEA (2.5:1, 42 mL). The OL (EtOAc/water) was washed with brine, dried, concentrated, and purified by FC (hexane/EtOAc 1:4) to give tert-butyl 4-[4-[bis-(trideuteriomethyl)-carbamoyl]phenyl]-3,6-dihydro-2H-pyridine-1-carboxylate (3.5 g). 2.5 g of this material was stirred ON in DCM/TFA (1:1, 30 mL) at 0° C. to RT and concentrated. The OL (aq. NaHCO₃/10% MeOH in DCM) was dried, concentrated, and triturated in Et₂O/pentane to give Int. 1AE7′ N,N-Bis(methyl-d₃)-4-(1,2,3,6-tetrahydropyridin-4-yl)benzamide (1.7 g).

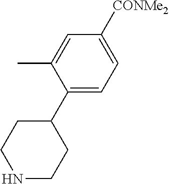

tert-Butyl 4-(4,4,5,5-tetramethyl-1,3,2-dioxaborolan-2-yl)-3,6-dihydropyridine-1(2H)-carboxylate (33.7 g), 4-bromo-N,N,3-trimethylbenzamide (20.0 g), Na₂CO₃ (21.4 g), and PdDPPFCl₂-DCM (1.48 g) were degassed in dioxane/water (9:1, 200 mL) and stirred ON at 110° C. The mixture was filtered, concentrated, and purified by FC (heptane/EtOAc 1:1) to give tert-butyl 4-[4-(dimethylcarbamoyl)-2-methyl-phenyl]-3,6-dihydro-2H-pyridine-1-carboxylate (25 g). 15 g of this material was hydrogenated ON using 10% Pd/C (5.0 g) in MeOH (200 mL), filtered, and concentrated to give tert-butyl 4-[4-(dimethylcarbamoyl)-2-methyl-phenyl]-piperidine-1-carboxylate (14.0 g). 10 g of this material was stirred ON in DCM/TFA (1:1, 200 mL) at 0° C. to RT, concentrated, and triturated in Et₂O to give Int. 1AE8 N,N,3-trimethyl-(4-piperidin-4-yl)benzamide (9.2 g, TFA salt).

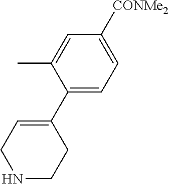

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE8′ N,N,3-trimethyl-4-(1,2,3,6-tetrahydropyridin-4-yl)benzamide (1.06 g, HCl salt).

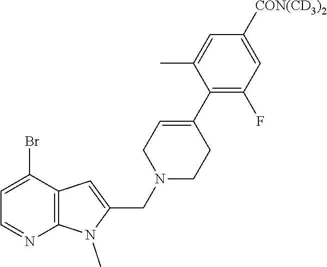

Ints. 1J1/1 (0.70 g/0.49 g, HCl salt) and KI (0.11 g) were stirred ON in DMF/DIPEA (8.3:1, 11.2 mL) at 0° C. to RT. The OL (EtOAc/water) was washed with brine, dried, concentrated, and purified by FC (EtOAc/hexane 7:3) to give Int. 1AE9 4-(1-((4-bromo-1-methyl-1H-pyrrolo[2,3-b]pyridin-2-yl)methyl)-1,2,3,6-tetrahydropyridin-4-yl)-3-fluoro-5-methyl-N,N-bis(methyl-d₃)benzamide (0.45 g).

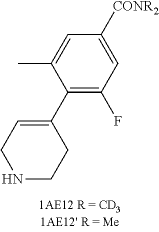

Methyl 4-bromo-3-fluoro-5-methyl-benzoate (20 g), tert-butyl 4-(4,4,5,5-tetramethyl-1,3,2-dioxaborolan-2-yl)-3,6-dihydropyridine-1(2H)-carboxylate (27.5 g), K₂CO₃ (44.8 g), and PdDPPFCl₂-DCM (3.31 g) were degassed in dioxane/water (4:1, 0.5 L) and stirred at 100° C. ON. The OL (water/EtOAc) was washed with brine, dried, concentrated, and purified by FC (heptane to EtOAc) to give tert-butyl 4-(2-fluoro-4-methoxy-carbonyl-6-methyl-phenyl)-3,6-dihydro-2H-pyridine-1-carboxylate (15.5 g). This material and LiOH—H₂O (3.72 g) were stirred ON in MeOH/water (1:1, 0.3 L) and acidified to precipitate 4-(1-tert-butoxycarbonyl-3,6-dihydro-2H-pyridin-4-yl)-3-fluoro-5-methyl-benzoic acid (11.8 g). 5.0 g of this material, HN(CD₃)₂ (1.44 g, HCl salt), and HATU (6.8 g) were stirred ON in DMF/DIPEA (6.4:1, 58 mL), concentrated, and purified by FC (hexane to EtOAc) to give tert-butyl 4-[4-[bis(trideuterio-methyl)carbamoyl]-2-fluoro-6-methyl-phenyl]-3,6-dihydro-2H-pyridine-1-carboxylate (5.1 g). This material was stirred 1 h in HFIP/10M aq. HCl (31:1, 52 mL) and diluted with MTBE to precipitate Int. 1AE12 3-Fluoro-5-methyl-N,N-bis(methyl-d₃)-4-(1,2,3,6-tetrahydropyridin-4-yl)benzamide (3.45 g, HCl salt). Int. 1AE12′ 3-fluoro-5-methyl-N,N-bis(methyl)-4-(1,2,3,6-tetrahydropyridin-4-yl)benzamide was prepared in a similar manner from HN(CH₃)₂.

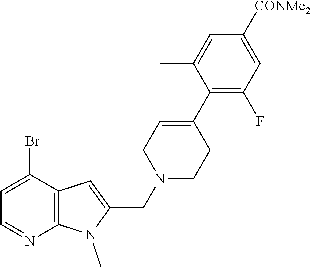

Int. 1J3 (0.50 g) was stirred 1 h in DCM (20 mL) and MsCl (0.8 mL) at 0° C. The OL (water/DCM) was washed with sat. aq. NaHCO₃ and concentrated to give (4-bromo-1-methyl-pyrrolo[2,3-b]pyridin-2-yl)methyl methanesulfonate (0.52 g). 0.5 g of this material, KI (0.13 g), and Int. 1AE12′ (0.51 g) were stirred ON in DMF/DIPEA (14:1, 21.5 mL) at 0° C. to RT. The OL (water/EtOAc) was dried, concentrated, and purified by FC (hexane to EtOAc) to give Int. 1AE13 4-(1-((4-Bromo-1-methyl-1H-pyrrolo[2,3-b]pyridin-2-yl)methyl)-1,2,3,6-tetrahydropyridin-4-yl)-3-fluoro-N,N,5-trimethyl-benzamide (0.52 g).

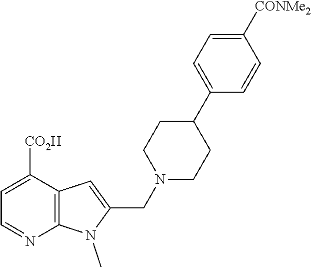

Int. 1AB1 (0.15 g), Mo(CO)₆ (87 mg), Pd₂dba₃ (8 mg), and XantPhos (10 mg) were degassed in EtOH (5 mL) and stirred 0.5 h at 120° C. under MW conditions. The mixture was filtered and HPLC-purified give ethyl 2-((4-(4-(dimethylcarbamoyl)phenyl)piperidin-1-yl)methyl)-1-methyl-1H-pyrrolo[2,3-b]pyridine-4-carboxylate (0.25 g). This material and LiOH (69 mg) were stirred in water/EtOH (2:1, 1.5 mL), concentrated, and HPLC-purified to give Int. 1AE15 2-((4-(4-(Dimethylcarbamoyl)phenyl)piperidin-1-yl)methyl)-1-methyl-1H-pyrrolo[2,3-b]pyridine-4-carboxylic acid (0.15 g).

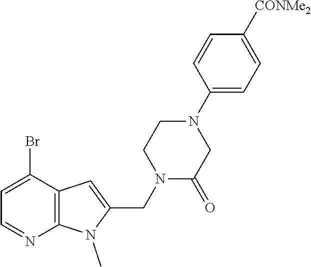

Piperazin-2-one (1.0 g), 4-bromo-N,N-dimethylbenzamide (1.14 g), K₃PO₄ (6.4 g), Pd(OAc)₂ (0.22 g), and RuPhos (0.93 g) were degassed in tert-butyl alcohol (15 mL) and stirred ON at 80-90° C. The mixture was filtered, concentrated, and purified by FC (DCM/MeOH 9:1) to give N,N-dimethyl-4-(3-oxopiperazin-1-yl)benzamide (0.70 g). 0.25 g of this material and NaH (81 mg) were stirred 1 h in THF/DMF (10:1, 5.5 mL) at 0° C. to RT. Int. 1J1 (0.25 g) was added and stirring continued ON at 0° C. to RT. The OL (10% MeOH in DCM/water) was dried, concentrated, and purified by FC (DCM/MeOH 9:1) to give Int. 1 AE16 4-(4-((4-Bromo-1-methyl-1H-pyrrolo[2,3-b]pyridin-2-yl)methyl)-3-oxopiperazin-1-yl)-N,N-dimethylbenzamide (0.20 g).

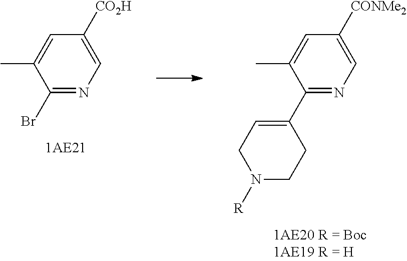

6-Bromo-5-methyl-pyridine-3-carboxylic acid (4.5 g), HATU (9.5 g), and HN(CH₃)₂ (8.4 g, HCl salt) were stirred ON in DMF/DIPEA (2.5:1, 45 mL) at 0° C. to RT. The OL (water/EtOAc) was dried, concentrated and purified by FC (hexane/EtOAc 3:7) to give Int. 1AE21 6-bromo-N,N,5-trimethylnicotinamide (4.2 g). 3.2 g of this material, NaHCO₃ (3.9 g), tert-butyl 4-(4,4,5,5-tetramethyl-1,3,2-dioxaborolan-2-yl)-3,6-dihydro-pyridine-1(2H)-carboxylate (6.1 g), and PdDPPFCl₂-DCM (0.27 g) were degassed in dioxane/water (3.7:1, 14 mL) and stirred ON at 110° C. The mixture was filtered, concentrated, and purified by FC (hexane/EtOAc 1:4) to give Int. 1AE20 tert-butyl 5-(dimethylcarbamoyl)-3-methyl-3′,6′-dihydro-[2,4′-bipyridine]-1′(2′H)-carboxylate (4.0 g). 0.25 g of this material was stirred ON in DCM/TFA (8.4:1, 5.6 mL) at 0° C. to RT, concentrated, and triturated in Et₂O/pentane to give Int. 1AE19 N,N,5-trimethyl-6-(1,2,3,6-tetrahydro-pyridin-4-yl)pyridine-3-carboxamide (0.28 g, TFA salt).

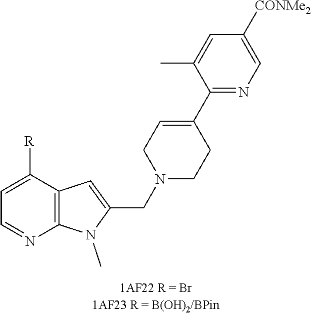

Ints. 1AE19/1J1 (0.5 g/0.5 g) were stirred ON ACN/Et₃N (18:1, 26 mL), concentrated, and HPLC-purified to give Int. 1AE22 6-[1-[(4-bromo-1-methyl-pyrrolo[2,3-b]pyridin-2-yl)methyl]-3,6-dihydro-2H-pyridin-4-yl]-N,N,5-trimethyl-pyridine-3-carboxamide (0.3 g). Int. 1AE22 (3 g), B₂Pin₂ (5.0 g), KOAc (5.0 g), and PdDPPFCl₂-DCM (0.05 g) were degassed in dioxane (25 mL) and stirred ON at 80° C. The OL (water/EtOAc) was dried and concentrated to give Int. 1AE23 (2-((5-(dimethylcarbamoyl)-3-methyl-3′,6′-dihydro-[2,4′-bipyridin]-1′(2′H)-yl)methyl)-1-methyl-1H-pyrrolo[2,3-b]pyridin-4-yl)boronic acid/N,N,3-trimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide (1.0 g).

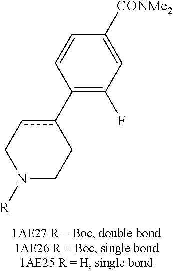

4-Bromo-3-fluoro-N,N-dimethyl-benzamide (1.0 g), tert-butyl 4-(4,4,5,5-tetramethyl-1,3,2-dioxaborolan-2-yl)-3,6-dihydro-2H-pyridine-1-carboxylate (1.51 g), Na₂CO₃ (1.72 g), and PdDPPFCl₂-DCM (0.17 g) were degassed in dioxane/water (4:1, 10 mL) and stirred at 100° C. ON. The OL (water/EtOAc) was washed with brine, dried, concentrated, and purified by FC (hexane to EtOAc) to give Int. 1AE27 tert-butyl 4-[4-(dimethyl-carbamoyl)-2-fluoro-phenyl]-3,6-dihydro-2H-pyridine-1-carboxylate (1.2 g). This material was hydrogenated ON using 10% Pd/C (40 mg) in MeOH (10 mL) under an atmosphere of H₂ in a sealed tube, filtered, and concentrated to give Int. 1AE26 tert-butyl 4-[4-(dimethylcarba-moyl)-2-fluoro-phenyl]piperidine-1-carboxylate (0.60 g) that was stirred in 4M HCl in dioxane (10 mL) ON, concentrated, and HPLC-purified to give Int. 1AE25 3-fluoro-N,N-dimethyl-4-(piperidin-4-yl)benzamide (63 mg).

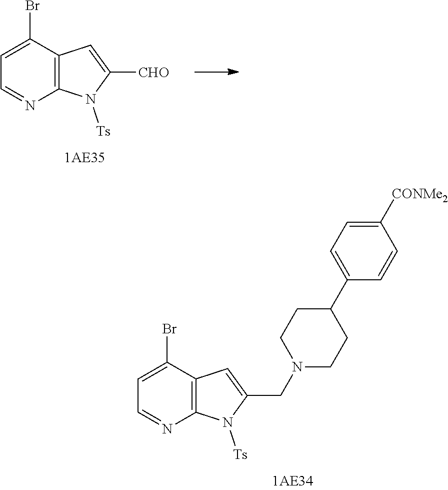

4-Bromo-1-tosyl-1H-pyrrolo[2,3-b]pyridine (1.0 g) was stirred 1 h in 0.6 M LDA in THF (14 mL) at −78° C. DMF (1.3 mL) was added and stirring continued 2 h at 78° C. The OL (sat. aq. NH₄Cl/EtOAc) was dried, concentrated, and purified by FC (hexane/EtOAc 9:1) to give Int. 1AE35 4-bromo-1-(p-tolyl-sulfonyl)pyrrolo[2,3-b]pyridine-2-carbaldehyde (0.6 g). Int. 1A35 (1.5 g) and N,N-dimethyl-4-(piperidin-4-yl)benzamide (1.6 g, HCl salt) were stirred ON in DCM/DIPEA (5:1, 24 mL). STAB (2.5 g) was added and stirring continued 2 h at 0° C. to RT. The OL (DCM/water) was dried, concentrated, and purified by FC (DCM/MeOH 9:1) to give Int. 1AE34 4-[1-[[4-bromo-1-(p-tolylsulfonyl)-pyrrolo[2,3-b]pyridin-2-yl]methyl]-4-piperidyl]-N,N-dimethyl-benzamide (1.0 g).

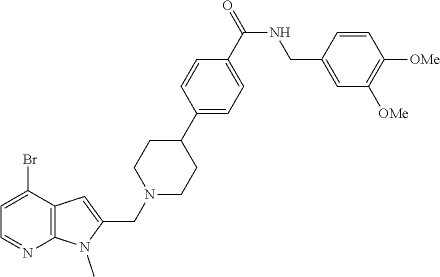 [SMILES: COc1ccc(CNC(=O)c2ccc(C3CCN(Cc4cc5c(Br)ccnc5n4C)CC3)cc2)cc1OC]

Int. 1AC4 (0.10 g), HATU (164 mg), and (3,4-dimethoxybenzyl)(methyl)-amine (58 mg) were stirred 3 h in DMF/DIPEA (25:1, 3.1 mL). The OL (sat. aq. NaHCO₃/DCM) was dried, concentrated, and purified by FC (DCM to DCM/MeOH 93:7) to give Int. 1AE36 4-(1-((4-bromo-1-methyl-1H-pyrrolo-[2,3-b]pyridin-2-yl)methyl)piperidin-4-yl)-N-(3,4-dimethoxybenzyl)-N-methylbenzamide (0.12 g).

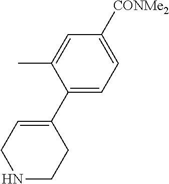

tert-Butyl 4-[4-(dimethylcarbamoyl)-2-methyl-phenyl]-3,6-dihydro-2H-pyridine-1-carboxylate (4.0 g) was stirred 0.5 h in 4M HCl in dioxane (40 mL), concentrated, and triturated in Et₂O to give Int. 1AE38 N,N,3-trimethyl-4-(1,2,3,6-tetrahydropyridin-4-yl)benzamide (1.06 g, HCl salt).

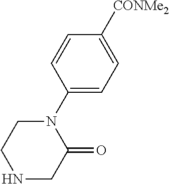

tert-Butyl 3-oxopiperazine-1-carboxylate (0.50 g), 4-bromo-N,N-dimethylbenzamide (0.68 g), K₃PO₄ (1.06 g), CuI (71 mg), DMDCH (36 mg), were degassed in dioxane (10 mL) and was stirred at 100° C. ON. The OL (water/EtOAc) was dried, concentrated, and purified by FC (EtOAc/MeOH 1:0 to 9:1) to give tert-butyl 4-(4-(dimethylcarbamoyl)-phenyl)-3-oxopiperazine-1-carboxylate (0.80 g). 0.23 g of this material was stirred 1 h in DCM/TFA (1:1, 6 mL) and concentrated to give Int. 1AE39 N,N-Dimethyl-4-(2-oxo-piperazin-1-yl)benzamide (0.24 g, TFA salt).

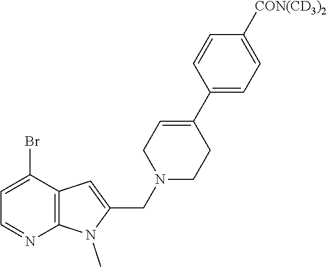

Int. 1J1/1AE7′ (0.33 g/0.30 g), and KI (21 mg) were stirred ON in DMF/DIPEA (7.3:1, 9 mL) at 0° C. to RT. The OL (water/EtOAc) was dried, concentrated, and purified by FC (hexane to EtOAc) to give Int. 1AF1 4-(1-((4-Bromo-1-methyl-1H-pyrrolo[2,3-b]pyridin-2-yl)methyl)-1,2,3,6-tetrahydropyridin-4-yl)-N,N-bis(methyl-d₃)benzamide (0.20 g).

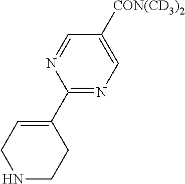

Methyl 2-bromopyrimidine-5-carboxylate (10.0 g), tert-butyl 4-(4,4,5,5-tetramethyl-1,3,2-dioxaborolan-2-yl)-3,6-dihydropyridine-1(2H)-carboxylate (15.6 g), K₂CO₃ (25.5 g), and PdDPPFCl₂-DCM (1.88 g) were degassed in dioxane/water (4:1, 0.25 L) and stirred ON at 100° C. The OL (water/EtOAc) was washed with brine, dried, concentrated, and purified by FC (hexane to EtOAc) to give methyl 2-(1-tert-butoxycarbonyl-3,6-dihydro-2H-pyridin-4-yl)pyrimidine-5-carboxylate (14.7 g). This material and LiOH—H₂O (2.15 g) were stirred ON in MeOH/water (1:1, 200 mL) and acidified to precipitate 2-(1-tert-butoxy-carbonyl-3,6-dihydro-2H-pyridin-4-yl)pyrimidine-5-carboxylic acid (8.0 g). 4.0 g of this material, HN(CD₃)₂ (1.21 g, HCl salt), and HATU (5.27 g) were stirred in DMF/DIPEA (3:1, 27 mL), concentrated, and purified by FC (hexane to EtOAc) to give tert-butyl 4-[5-[bis(tri-deuteriomethyl)carbamoyl]pyrimidin-2-yl]-3,6-dihydro-2H-pyridine-1-carboxylate (2.20 g). This material was stirred in 1 h HFIP/10M aq. HCl (30:1, 23 mL), concentrated, and triturated in MTBE to give Int. 1AF2 N,N-bis(methyl-d₃)-2-(1,2,3,6-tetrahydropyridin-4-yl)pyrimidine-5-carboxamide (1.40 g, HCl salt).

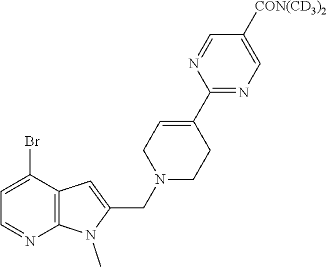

Ints. 1AF2/1J1 (0.5 g/0.5 g) were stirred in ACN/Et₃N (19:1, 26 mL), concentrated, and HPLC-purified to give Int. 1AF3 2-[1-[(4-Bromo-1-methyl-pyrrolo[2,3-b]pyridin-2-yl)methyl]-3,6-dihydro-2H-pyridin-4-yl]-N,N-bis(trideuteriomethyl)pyrimidine-5-carboxamide (0.3 g).

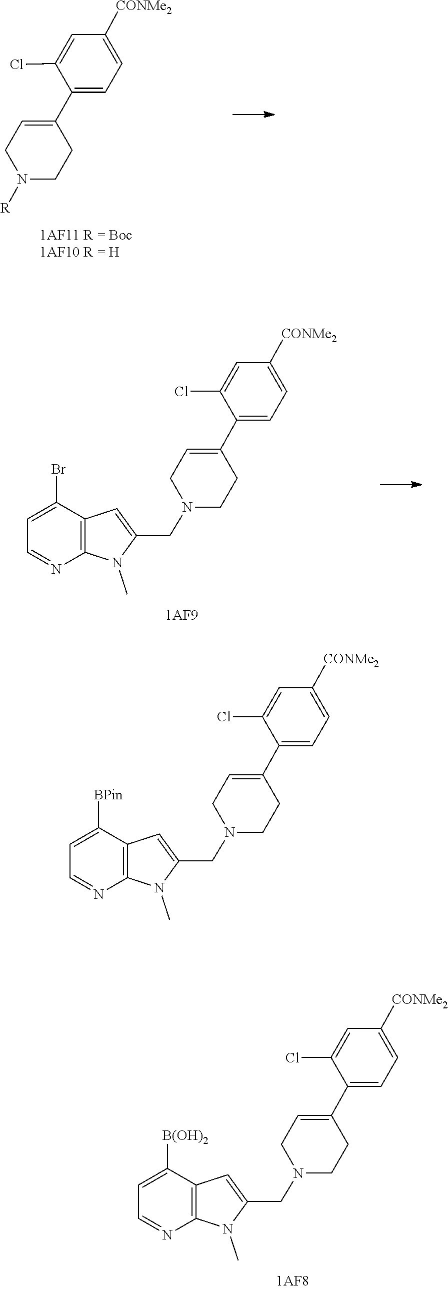

4-Bromo-3-fluoro-N,N-dimethyl-benzamide (1 g), tert-butyl 4-(4,4,5,5-tetramethyl-1,3,2-dioxaborolan-2-yl)-3,6-dihydropyridine-1(2H)-carboxylate (1.5 g), Na₂CO₃ (1.7 g), PdPDDFCl₂-DCM (0.17 g) were deassed in dioxane/water (4:1, 10 mL) and stirred ON at 100° C. The OL (water/EtOAc) was washed with brine, dried, concentrated, and purified by FC (hexane to EtOAc) to give Int. 1AF11 tert-butyl 4-[4-(dimethylcarbamoyl)-2-fluoro-phenyl]-3,6-dihydro-2H-pyridine-1-carboxylate (1.2 g). Int. 1AF11 (0.6 g) was stirred ON in 4M HCl in dioxane (10 mL). The residue after concentration was HPLC-purified to give Int. 1AF10 3-fluoro-N,N-dimethyl-4-(1,2,3,6-tetrahydropyridin-4-yl)benzamide (0.22 g). Ints. 1AF10/1J1 (0.5 g/0.5 g) were stirred ON in ACN/Et₃N (19:1, 26 mL), concentrated, and HPLC-purified to give Int. 1AF9 4-[1-[(4-bromo-1-methyl-pyrrolo[2,3-b]pyridin-2-yl)-methyl]-3,6-dihydro-2H-pyridin-4-yl]-N,N,3,5-tetramethyl-benzamide (0.3 g). Int. 1AF9 (3 g), B₂pin₂ (5 g), KOAc (5 g), and PdDPPFCl₂-DCM (50 mg) were degassed in dioxane and stirred ON at 80° C. The OL (EtOAc/water) was dried and concentrated to give Int. 1AF8 3-chloro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethylcarbamoyl)phenyl)-3,6-dihydropyridin-1(2H)-yl)methyl)-1-methyl-1H-pyrrolo[2,3-b]pyridin-4-yl)boronic acid (1 g).

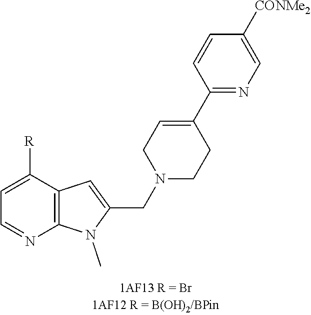

Ints. 1S5/1J1 (0.5 g/0.5 g, as the HCl salt) were stirred ON in ACN/Et₃N (19:1, 26 mL), concentrated, and HPLC-purified to give Int. 1AF13 6-[1-[(4-bromo-1-methyl-pyrrolo-[2,3-b]pyridin-2-yl)methyl]-3,6-dihydro-2H-pyridin-4-yl]-N,N-dimethyl-pyridine-3-carboxamide (0.3 g). Int. 1AF13 (3.0 g), KOAc (5.0 g), B₂Pin₂ (5.0 g), and PdDPPFCl₂-DCM (50 mg) were degassed in dioxane (25 mL) and stirred at 80° C. ON. The OL (water/EtOAc) was concentrated to give Example 1AF12 N,N-Dimethyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxamide/(2-((5-(dimethylcarbamoyl)-3′,6′-dihydro-[2,4′-bipyridin]-1′(2′H)-yl)methyl)-1-methyl-1H-pyrrolo[2,3-b]pyridin-4-yl)boronic acid (1.0 g).

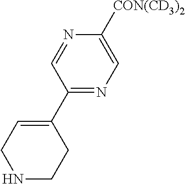

Methyl 5-bromopyrazine-2-carboxylate (11.0 g), tert-butyl 4-(4,4,5,5-tetramethyl-1,3,2-dioxaborolan-2-yl)-3,6-dihydropyridine-1(2H)-carboxylate (17.2 g), K₂CO₃ (28.0 g), PdPDDFCl₂-DCM (2.07 g) were deassed in dioxane/water (5:1, 250 mL) and stirred ON at 100° C. The aq. layer (water/EtOAc) was acidified to precipitate 5-(1-tert-butoxy-carbonyl-3,6-dihydro-2H-pyridin-4-yl)pyrazine-2-carboxylic acid (12.0 g). 4.5 g of this material, HN(CD₃)₂ (1.40 g, HCl salt), and HATU (6.20 g) were stirred ON in DMF/DIPEA (2.6:1, 28 mL), concentrated, and purified by FC (hexane to EtOAc) to give tert-butyl 4-[5-[bis(trideuteriomethyl)-carbamoyl]pyrazin-2-yl]-3,6-dihydro-2H-pyridine-1-carboxylate (2.0 g). This material was stirred in 1 h HFIP/10M aq. HCl (29:1, 21 mL), concentrated, and triturated in MTBE to give Int. 1AF14 5-(1,2,3,6-Tetrahydropyridin-4-yl)-N,N-bis(trideuteriomethyl)pyrazine-2-carboxamide (0.88 g, HCl salt).

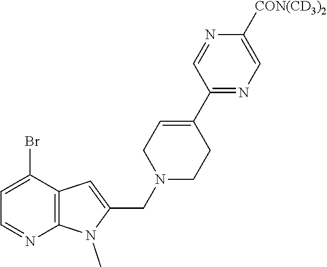

Ints. 1AF14/1J1 (0.5 g/0.5 g), and Et₃N (1.2 mL) were stirred ON in ACN (25 mL), concentrated, and HPLC-purified to give Int. 1AF15 5-(1-((4-Bromo-1-methyl-1H-pyrrolo[2,3-b]pyridin-2-yl)methyl)-1,2,3,6-tetrahydropyridin-4-yl)-N,N-bis(methyl-d₃)pyrazine-2-carboxamide (0.3 g).

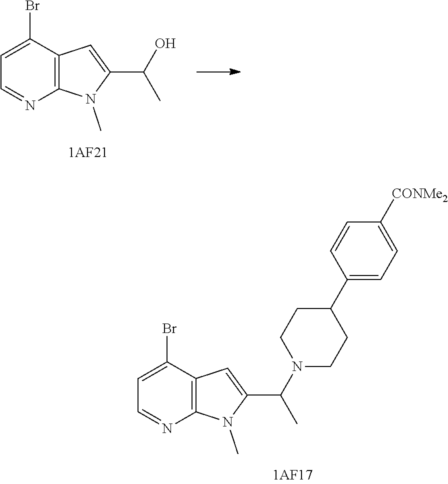

1.0M MeMgBr in THF (185 mL) was added to a solution of Int. 1AB8 (29.5 g) in THF (1.2 L) at 0° C. and stirring continued 3 h. The OL (sat. aq. NH₄Cl/EtOAc) was washed with brine, dried, concentrated and purified by FC (pentane/EtOAc 3:1) to give Int. 1AF21 1-(4-bromo-1-methyl-1H-pyrrolo[2,3-b]pyridin-2-yl)ethan-1-ol (25.5 g). 0.30 g of this material was stirred in toluene/SOCl₂ (12.5:1, 10.8 mL) at 0° C. before stirring 0.5 h at 110° C. The mixture was concentrated. The residue, Cs₂CO₃ (1.15 g), KI (0.98 g), and N,N-dimethyl-4-(4-piperidyl)-benzamide (0.41 g) were stirred ON in ACN (10 mL) at 0° C. to RT. The OL (water/EtOAc) was washed with brine, dried, concentrated, and purified by FC (DCM/MeOH 95:5) to give Int. 1AF17 4-(1-(1-(4-bromo-1-methyl-1H-pyrrolo[2,3-b]pyridin-2-yl)ethyl)-piperidin-4-yl)-N,N-dimethylbenzamide (0.28 g).

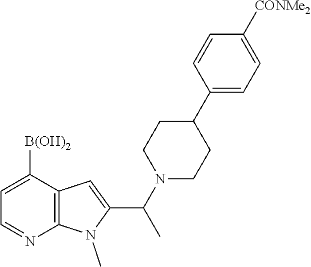

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl)piperidin-1-yl)ethyl)-1-methyl-1H-pyrrolo[2,3-b]pyridin-4-yl)boronic acid (0.85 g).

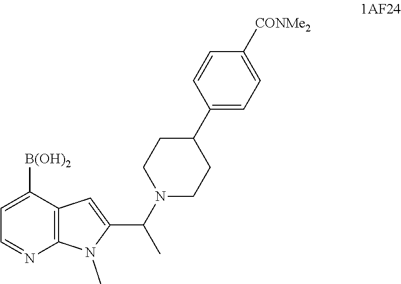

Int. 1AF17 (0.70 g), B₂Pin₂ (0.51 g), KOAc (0.4 g), and Pd(PPh₃)₂Cl₂ (0.1 g) were degassed in THF (50 mL) and stirred ON at 100° C., filtered, concentrated, and triturated in pentane to give Int. 1AF24 (2-(1-(4-(4-(dimethyl-carbamoyl)phenyl)piperidin-1-yl)ethyl)-1-methyl-1H-pyrrolo[2,3-b]pyridin-4-yl)boronic acid (0.85 g).

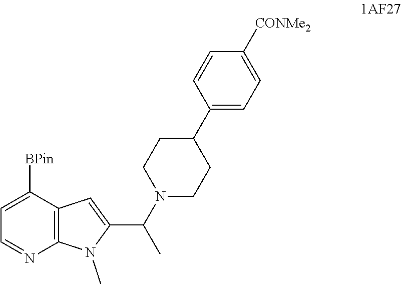

Int. 1AF17 (0.70 g), B₂Pin₂ (38 mg), KOAc (45 mg), and PdDPPFCl₂-DCM (25 mg) were degassed in dioxane (10 mL) and stirred ON at 110° C. The mixture was filtered, concentrated, and purified by FC (pentane/EtOAc 95:5) to give Int. 1AF27 N,N-dimethyl-4-(1-(1-(1-methyl-4-(4,4,5,5-tetramethyl-1,3,2-dioxaborolan-2-yl)-1H-pyrrolo-[2,3-b]pyridin-2-yl)ethyl)piperidin-4-yl)benzamide (0.85 g).

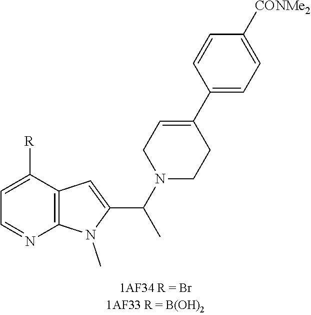

1-(4-Bromo-1-methyl-1H-pyrrolo[2,3-b]pyridin-2-yl)ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂(PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethylcarbamoyl)-phenyl)-3,6-dihydropyridin-1(2H)-yl)ethyl)-1-methyl-1H-pyrrolo[2,3-b]pyridin-4-yl)-boronic acid (0.50 g).

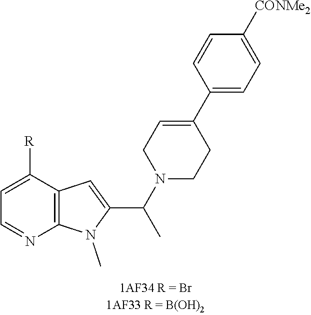

1-(4-Bromo-1-methyl-1H-pyrrolo[2,3-b]pyridin-2-yl)ethan-1-ol (0.20 g) was stirred 1.5 h in toluene/SOCl₂ (29:1, 5.2 mL) at 0° C. to 110° C. and concentrated. The residue, KI (10 mg), and Int. 1AF5 (0.23 g) were stirred ON in DMF/DIPEA (2.5:1, 2.8 mL) at 0° C. to RT. The OL (water/EtOAc) was washed with brine, dried, concentrated, and purified by FC (hexane/EtOAc 9:1 to 85:15) to give Int. 1AF34 4-[1-[1-(4-bromo-1-methyl-pyrrolo-[2,3-b]pyridin-2-yl)ethyl]-3,6-dihydro-2H-pyridin-4-yl]-N,N-dimethyl-benzamide (80 mg). Int. 1AF34 (0.32 g), B₂Pin₂ (24 mg), KOAc (25 mg), and PdCl₂(PPh₃)₂ (48 mg) were degassed in THF (5 mL) and stirred ON at 90° C. The mixture was filtered, concentrated, and purified by FC (pentane/EtOAc 95:5) to give Int. 1AF33 (2-(1-(4-(4-(dimethyl-carbamoyl)-phenyl)-3,6-dihydropyridin-1(2H)-yl)ethyl)-1-methyl-1H-pyrrolo[2,3-b]pyridin-4-yl)-boronic acid (0.50 g).

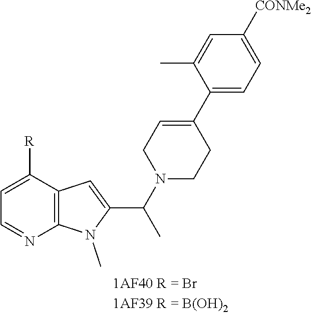

Int. 1AF40 4-[1-[1-(4-bromo-1-methyl-pyrrolo[2,3-b]pyridin-2-yl)ethyl]-3,6-dihydro-2H-pyridin-4-yl]-N,N,3-trimethyl-benzamide (0.15 g) was prepared similarly to Int. 1AF17 from Int. 1AF21 (0.20 g) and Int. 1AE38 (0.37 g, TFA salt). Int. 1AF39 (2-(1-(4-(4-(dimethylcarbamoyl)-2-methylphenyl)-3,6-dihydropyridin-1(2H)-yl)-ethyl)-1-methyl-1H-pyrrolo[2,3-b]pyridin-4-yl)boronic acid (40 mg) was prepared similarly to Int. 1AF33 from Int. 1AF40 (50 mg).

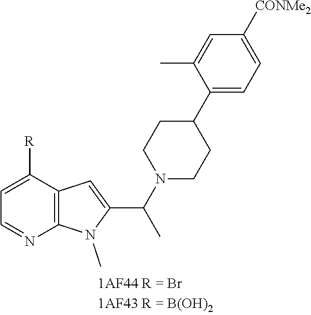

Int. 1AF44 4-[1-[1-(4-bromo-1-methyl-pyrrolo[2,3-b]pyridin-2-yl)ethyl]-4-piperidyl]-N,N,3-trimethyl-benzamide (0.15 g) was prepared similarly to Int. 1AF17 from Ints. 1AF24/1AE38 (0.20 g/0.42 g, TFA salt). Int. 1AF43 (2-(1-(4-(4-(dimethylcarbamoyl)-2-methylphenyl)-3,6-dihydropyridin-1(2H)-yl)ethyl)-1-methyl-1H-pyrrolo[2,3-b]-pyridin-4-yl)boronic acid (0.60 g) was prepared similarly to Int. 1AF39 from Int. 1AF44 (0.50 g).

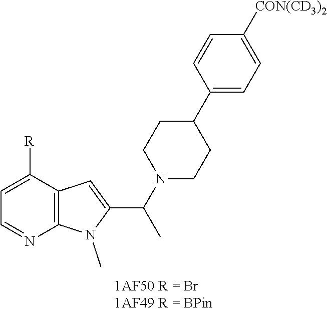

Int. 1AF50 4-[1-[1-(4-bromo-1-methyl-pyrrolo[2,3-b]pyridin-2-yl)ethyl]-4-piperidyl]-N,N-bis(trideuterio-methyl)benzamid (0.65 g) was prepared similarly to Int. 1AF17 from Ints. 1AF21/1D7 (0.35 g/0.53 g, TFA salt). Int. 1AF49 [2-[1-[4-[4-[bis(trideuterio-methyl)carbamoyl]phenyl]-1-piperidyl]ethyl]-1-methyl-pyrrolo[2,3-b]-pyridin-4-yl]-boronic acid (0.41 g) was prepared similarly to Int. 1AF25 from Int. 1AF50 (0.32 g) and B₂Pin₂ (0.34 g).

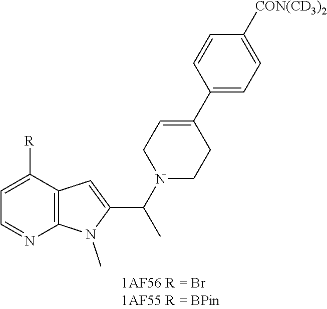

Int. 1AF56 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃)benzamide (0.60 g) was prepared similarly to Int. 1AF34 from Int. 1AF21 (0.50 g) and Int. 1AE7′ (0.69 g). Int. 1AF55 [2-[1-[4-[4-[bis(trideuteriomethyl)carbamoyl]phenyl]-3,6-dihydro-2H-pyridin-1-yl]ethyl]-1-methyl-pyrrolo[2,3-b]pyridin-4-yl]boronic acid (0.55 g) was prepared similarly to Int. 1AF39 from Int. 1AF56 (0.50 g).

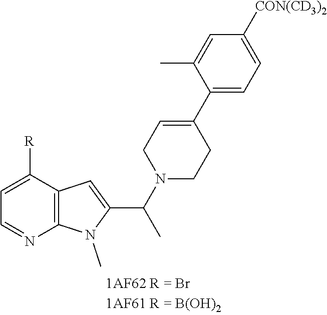

Int. 1AF62 4-(1-(1-(4-bromo-1-methyl-1H-pyrrolo[2,3-b]pyridin-2-yl)ethyl)-1,2,3,6-tetrahydro-pyridin-4-yl)-3-methyl-N,N-bis(methyl-d₃)benzamide (0.46 g) was prepared similarly to Int. 1AF17 from Ints. 1AF24/1S20 (0.50 g/0.70 g). Int. 1AF61 (2-(1-(4-(4-(bis(methyl-d₃)carbamoyl)-2-methylphenyl)-3,6-dihydropyridin-1(2H)-yl)ethyl)-1-methyl-1H-pyrrolo[2,3-b]pyridin-4-yl)boronic acid (0.75 g) was prepared similarly to Int. 1AF55 from B₂Pin₂ (0.52 g) and Int. 1AF62 (0.5 g).

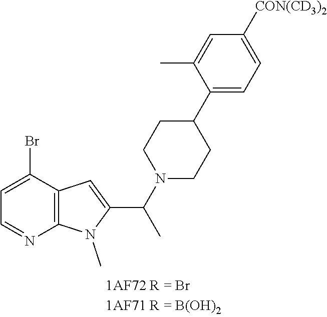

Int. 1AF72 4-(1-(1-(4-bromo-1-methyl-1H-pyrrolo[2,3-b]pyridin-2-yl)ethyl)piperidin-4-yl)-3-methyl-N,N-bis-(methyl-d₃)benzamide (90 mg) was prepared similarly to Int. 1AF17 from Ints. 1AF24/1S22 (0.10 g/0.19 g, TFA salt). Int. 1AF71 (2-(1-(4-(4-(bis(methyl-d₃)carbamoyl)-2-methylphenyl)piperidin-1-yl)ethyl)-1-methyl-1H-pyrrolo[2,3-b]pyridin-4-yl)boronic acid (0.10 g) was prepared similarly to Int. 1AF55 from Int. 1AF72 (90 mg) and B₂Pin₂ (0.12 g).

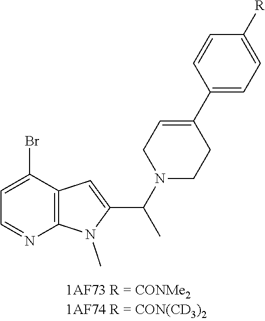

Int. 1AF21 (1.20 g) was stirred 1 h in toluene/SOCl₂ (5.9:1, 23.5 mL) at 0° C. to 110° C. and concentrated. The residue, Cs₂CO₃ (4.60 g), KI (0.39 g), and Int. 1AE7 (1.30 g) were stirred ON in ACN (30 mL) at 0° C. to RT. The OL (water/EtOAc) was washed with brine, dried, concentrated, and purified by FC (hexane/EtOAc 9:1 to 85:15) to give Int. 1AF73 4-[1-[1-(4-bromo-1-methyl-pyrrolo[2,3-b]pyridin-2-yl)ethyl]-3,6-dihydro-2H-pyridin-4-yl]-N,N-dimethyl-benzamide (1.0 g). Int. 1AF74 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃)benzamide (0.60 g) was prepared similarly from Ints. 1AE7′/1AF21 (0.69 g/0.50 g).

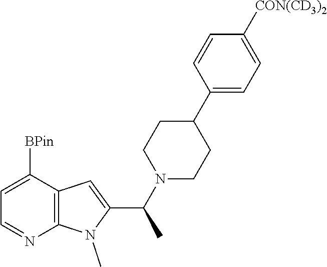

Int. 1AF76 (1.50 g), B₂Pin₂ (1.60 g), KOAc (0.93 g), and PdPDDFCl₂-DCM (0.26 g) were degassed in dioxane (30 mL) and stirred 6 h at 90° C., filtered, concentrated, and triturated in Et₂O to give Int. 1AF75 (S)—N,N-bis(methyl-d₃)-4-(1-(1-(1-methyl-4-(4,4,5,5-tetramethyl-1,3,2-dioxaborolan-2-yl)-1H-pyrrolo[2,3-b]pyridin-2-yl)ethyl)-piperidin-4-yl)benzamide (1.30 g).

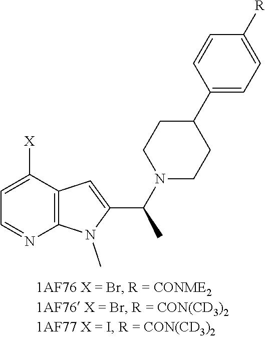

Int. 2C36 (0.35 g), HATU (0.3 g), and HN(CD₃)₂ (77 mg, HCl salt) were stirred ON in DMF/DIPEA (14.7:1, 10.7 mL) at 0° C. to RT and diluted with water to precipitate Int. 1AF76 (0.30 g). Int. 1AF76′ (3.5 g) was prepared similarly from Int. 2C36 (4.5 g). Int. 1AF76′ (0.10 g), CuI (20 mg), NaI (63 mg), and DMDCH (30 mg) were degassed in dioxane (5 mL) and stirred at 110° C. ON and filtered. The OL (DCM/aq. NH₃) was dried, concentrated, and purified by FC (DCM/MeOH 97:3) to give Int. 1AF77 (S)-4-(1-(1-(4-iodo-1-methyl-1H-pyrrolo[2,3-b]pyridin-2-yl)ethyl)piperidin-4-yl)-N,N-bis(methyl-d₃)-benzamide (50 mg). The absolute configuration of 1AF76 (S)-4-(1-(1-(4-bromo-1-methyl-1H-pyrrolo-[2,3-b]pyridin-2-yl)-ethyl)piperidin-4-yl)-N,N-dimethylbenzamide and 1AF76′ (S)-4-(1-(1-(4-bromo-1-methyl-1H-pyrrolo-[2,3-b]pyridin-2-yl)ethyl)-piperidin-4-yl)-N,N-bis(methyl-d₃)benzamide was determined to S by VCD.

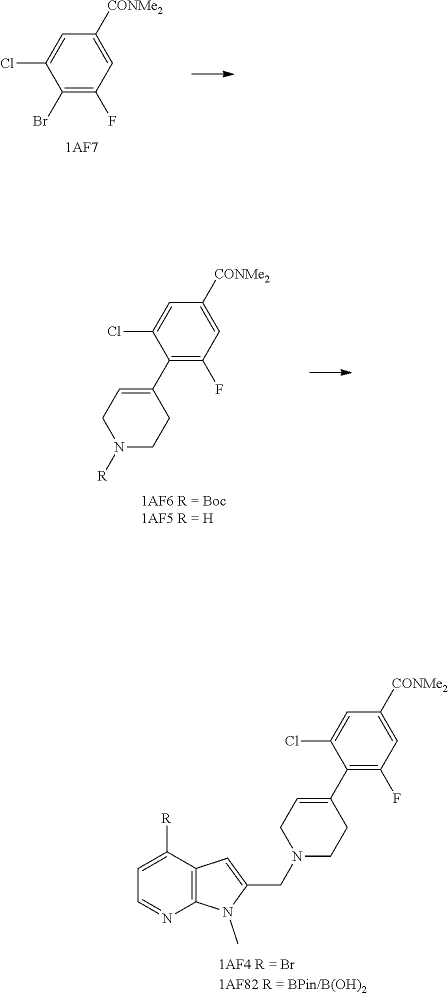

CDI (7.04 g) was added to a solution of 4-bromo-3-fluoro-5-methyl-benzoyl chloride (11.0 g) in DCM (50 mL) at 0° C. and stirring continued 0.5 h. HN(CH₃)₂ (7.1 g, HCl salt) was added before refluxing 5 h. The mixture was washed with water, dried, and concentrated to give Int. 1AF7 4-bromo-3-fluoro-N,N,5-trimethyl-benzamide (12.4 g). This material, tert-butyl 4-(4,4,5,5-tetramethyl-1,3,2-dioxa-borolan-2-yl)-3,6-dihydro-2H-pyridine-1-carboxylate (15.0 g), K₂CO₃ (18.3 g), and PdDPPFCl₂-DCM (0.73 g) were degassed in dioxane (100 mL) and stirred 48 h at 90° C. The OL (water/MTBE) was dried and concentrated to give Int. 1AF6 tert-butyl 4-(2-chloro-4-(dimethylcarbamoyl)-6-fluorophenyl)-3,6-dihydropyridine-1(2H)-carboxylate (11.0 g). Int. 1AF6 (0.6 g) was stirred 0.5 h in THF/4M HCl in dioxane (1:1, 2 mL). The OL (water/EtOAc) was dried and concentrated to give Int. 1AF5 3-chloro-5-fluoro-N,N-dimethyl-4-(1,2,3,6-tetrahydro-pyridin-4-yl)benzamide (0.25 g, HCl salt). This material, K₂CO₃ (0.37 g), and Int. 1J1 (0.23 g) were stirred in DMF (5 mL) ON. The OL (water/MTBE) was layer was dried and concentrated to give Int. 1AF4 4-[1-[(4-bromo-1-methyl-pyrrolo[2,3-b]pyridin-2-yl)-methyl]-3,6-dihydro-2H-pyridin-4-yl]-3-chloro-5-fluoro-N,N-dimethyl-benzamide (0.17 g). This material, B₂Pin₂ (0.17 g), KOAc (0.10 g), and PdDPPFCl₂-DCM (6 mg) were degassed in dioxane (5 mL) and stirred 48 h at 100° C. The OL (water/MTBE) was layer was dried and concentrated to give Int. 1AF82 3-Chloro-5-fluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide/(2-((4-(2-chloro-4-(dimethyl-carbamoyl)-6-fluorophenyl)-3,6-dihydropyridin-1(2H)-yl)methyl)-1-methyl-1H-pyrrolo[2,3-b]pyridin-4-yl)-boronic acid (0.19 g).

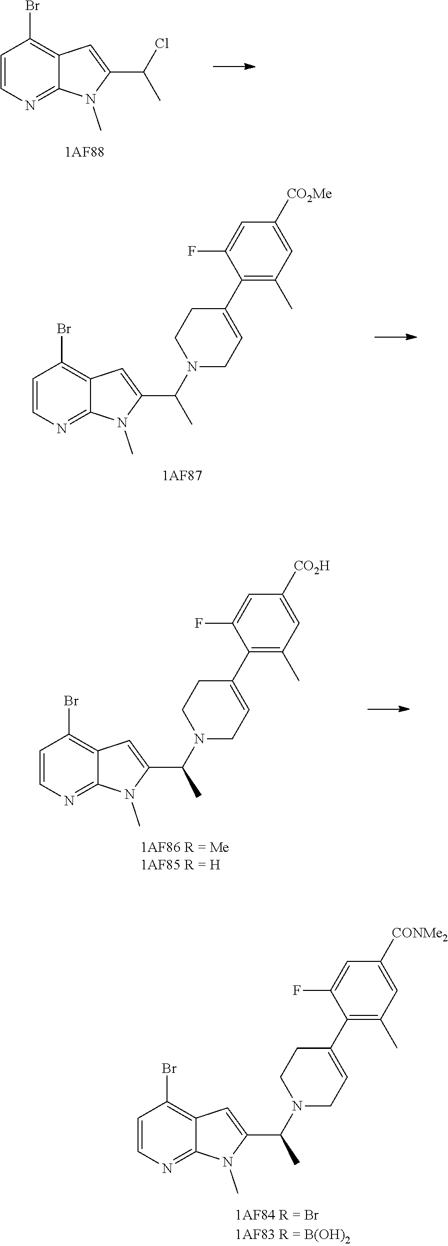

Int. 1AF21 (8 g) was stirred 0.5 h in toluene/SOCl₂ (4:1, 123 mL) at 0° C. to 80° C. and concentrated to give Int. 1AF88 (7.5 g). Ints. 1AF88/1AF90 (7.5 g, 7.8 g, HCl salt), Cs₂CO₃ (31 g), and KI (23 g) were stirred ON in ACN (100 mL) at 0° C. to RT. The mixture was filtered, concentrated, and purified by FC (hexane/EtOAc 41:9) to give Int. 1AF87 methyl 4-[1-[1-(4-bromo-1-methyl-pyrrolo[2,3-b]pyridin-2-yl)ethyl]-3,6-dihydro-2H-pyridin-4-yl]-3-fluoro-5-methyl-benzoate (3.9 g). Int. 1AF87 (0.2 g) was resolved by SFC using a Chiralpak AD-H 250×21 mm 5 μm column operated at 30° C. and an eluent of 70% CO₂ and 30% MeOH (70 g/min) and a back pressure of 100 bar to give Int. 1AF86 methyl (S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoate (70 mg, second peak). The absolute configuration was determined by VCD for Int. 1AF86. Int. 1AF86 (2.5 g) and LiOH (1.1 g) were stirred 1 h in THF/MeOH/water (2:1:1, 20 mL) at 0° C. to RT. The mixture was concentrated and triturated in aq. citric acid to precipitate Int. 1AF85 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (2.3 g). 4-[1-[(1S)-1-(4-bromo-1-methyl-pyrrolo[2,3-b]pyridin-2-yl)ethyl]-3,6-dihydro-2H-pyridin-4-yl]-3-fluoro-5-methyl-benzoic acid (1.8 g), HATU (2.2 g), and HNMe₂ (0.93 g, HCl salt) were stirred ON in DMF/DIPEA (6.1:1, 23.3 mL) at 0° C. to RT. The mixture was diluted with water to precipitate Int. 1AF84 4-[1-[(1S)-1-(4-bromo-1-methyl-pyrrolo[2,3-b]pyridin-2-yl)ethyl]-3,6-dihydro-2H-pyridin-4-yl]-3-fluoro-N,N,5-trimethyl-benzamide (1.5 g). Int. 1AF84′ (S)-4-(1-(1-(4-bromo-1-methyl-1H-pyrrolo[2,3-b]pyridin-2-yl)ethyl)-1,2,3,6-tetrahydropyridin-4-yl)-3-fluoro-5-methyl-N,N-bis(methyl-d₃)benzamide was prepared similarly using the HCl salt of HN(CD₃)₂. Int. 1AF84 (1.1 g), KOAc (0.86 g), B₂Pin₂ (1.1 g), and PdDPPFCl₂-DCM (0.18 g) stirred 1 h in dioxane (10 mL) at 110° C. under MW conditions. The mixture was filtered, concentrated, and triturated in pentane to give Int. 1AF83 [2-[(1S)-1-[4-[4-(dimethylcarbamoyl)-2-fluoro-6-methyl-phenyl]-3,6-dihydro-2H-pyridin-1-yl]ethyl]-1-methyl-pyrrolo[2,3-b]pyridin-4-yl]boronic acid (1.3 g).

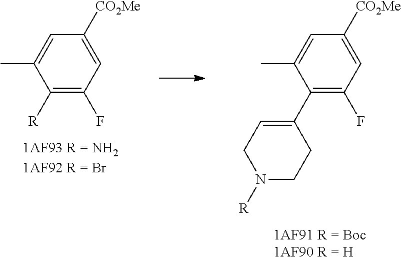

2-Fluoro-4-iodo-6-methyl-aniline (20 g) and PdDPPFCl₂-DCM were stirred ON in MeOH/Et₃N (23:1, 522 mL) under an atmosphere of CO (200 psi) at 80° C. The mixture was filtered. The OL (EtOAc/10% aq. EDTA) was dried and concentrated to afford Int. 1AF93 methyl 4-amino-3-fluoro-5-methyl-benzoate (14 g). Int. 1AF93 (25 g), tert-butyl nitrite (21.1 g), and CuBr₂ (152 g) were stirred ON in ACN (500 mL) at 0° C. to 80° C. The mixture was cooled to 0° C., diluted with sat. aq. NaHCO₃, and filtered. The OL (EtOAc/10% aq. NH₃) was concentrated and purified by FC (hexane/EtOAc 9:1) to give Int. 1AF92 methyl 4-bromo-3-fluoro-5-methyl-benzoate (21 g). Int. 1AF92 (1.0 g), tert-butyl 4-(4,4,5,5-tetramethyl-1,3,2-dioxaborolan-2-yl)-3,6-dihydropyridine-1(2H)-carboxylate (1.9 g), NaHCO₃ (1.29 g), and PdDPPFCl₂-DCM (0.33 g) were degassed in dioxane/water (5:1, 18 mL) and stirred ON at 110° C. The mixture was filtered. The OL (EtOAc/water) was dried, concentrated, and purified by FC (hexane/EtOAc 7:3) to give Int. 1AF91 tert-butyl 4-(2-fluoro-4-(methoxycarbonyl)-6-methyl-phenyl)-3,6-dihydro-pyridine-1(2H)-carboxylate (0.65 g). Int. 1AF91 (10 g) was stirred in 1.5 M HCl in dioxane (160 mL) at 0° C. to RT. The residue after concentration was triturated in Et₂O to give Int. 1AF90 methyl 3-fluoro-5-methyl-4-(1,2,3,6-tetrahydropyridin-4-yl)benzoate (13 g, HCl salt).

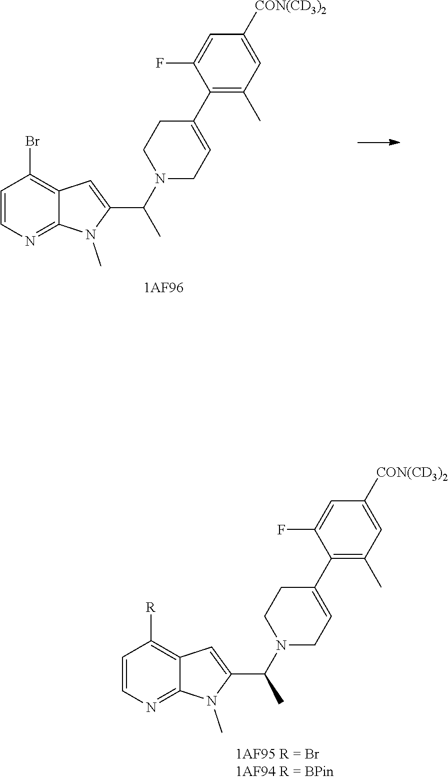

Ints. 1AF88/1AE12 (4.5 g, 5.5 g, HCl salt), Cs₂CO₃ (27 g), and KI (1.4 g) were stirred ON in ACN (50 mL) at 0° C. to RT. The mixture was diluted with water, filtered, and concentrated. The OL (EtOAc/water) was dried and concentrated. The residue was mixed with another batch prepared similarly on 1.5 g scale and purified by FC (pentane/EtOAc 3:7) to give Int. 1AF96 (4.8 g). Int. 1AF96 (5.0 g) was resolved by SFC using a Lux Cellulose.2 250×30 mm 5 μm column operated at 30° C. and an eluent of 70% CO₂ and 30% IPA (90 g/min) and a back pressure of 120 bar to give Int. 1AF95 (1.5 g, peak 2). Int. 1AF95 (0.5 g), B₂Pin₂ (0.38 g), KOAc (0.29 g), and PdDPPFCl₂-DCM (81 mg) were degassed in dioxane (5 mL) and stirred 1 h at 120° C. under MW conditions. The mixture was concentrated and triturated in pentane/Et₂O to give Int. 1AF94 3-fluoro-5-methyl-4-[1-[(1S)-1-[1-methyl-4-(4,4,5,5-tetramethyl-1,3,2-dioxaborolan-2-yl)pyrrolo[2,3-b]-pyridin-2-yl]ethyl]-3,6-dihydro-2H-pyridin-4-yl]-N,N-bis(trideuteriomethyl)benzamide (0.6 g).

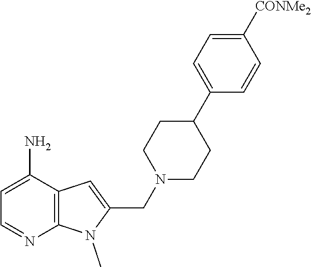

Int. 1AB1 (0.29 g), CuI (0.12 g), and NaN₃ (83 mg) were degassed in DMSO/DMEDA (14.4:1, 1.4 mL) and stirred at 100° C. The OL (sat. aq. NH₄Cl/EtOAc) was washed with water, brine, dried, concentrated, HPLC-purified, and triturated in MTBE to give Int. 1C1 4-[1-[(4-Amino-1-methyl-pyrrolo[2,3-b]pyridin-2-yl)methyl]-4-piperidyl]-N,N-dimethyl-benzamide (0.22 g).

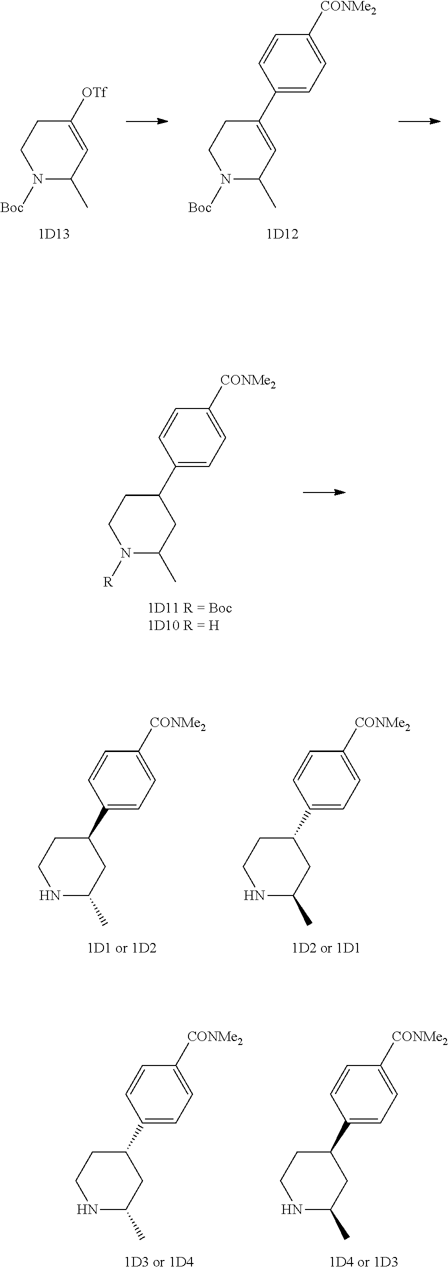

2.0M LDA in THF (28.1 mL) was added to a solution of tert-butyl 2-methyl-4-oxo-piperidine-1-carboxylate (10.0 g) in THF (200 mL) −78° C. The mixture was stirred for 0.3 h at −78° C. 1,1,1-Trifluoro-N-phenyl-N-((trifluoro-methyl)sulfonyl)methane-sulfonamide (20 g in THF (10 mL)) was added and stirring continued 3 h at −78° C. to RT. The OL (aq. NH₄Cl/EtOAc) was washed with water and brine, dried, and concentrated to give Int. 1D13 tert-butyl 6-methyl-4-(trifluoromethyl-sulfonyloxy)-3,6-dihydro-2H-pyridine-1-carboxylate (18.0 g). Int. 1D13 (10 g), Na₂CO₃ (7.67 g), (4-(dimethyl-carbamoyl)phenyl)boronic acid (8.38 g), and PdDPPFCl₂-DCM (1.20 g) were degassed in toluene/water (5:1, 120 mL) and refluxed ON. The OL (water/EtOAc) was dried, concentrated, and purified by FC (hexane/EtOAc 7:3) to give Int. 1D12 tert-butyl 4-[4-(dimethyl-carbamoyl)phenyl]-6-methyl-3,6-dihydro-2H-pyridine-1-carboxylate (5.2 g). 5.0 g of this material was hydrogenated ON using 10% Pd/C (2.50 g) and H₂ (50-60 psi) in EtOH (75 mL), filtered, concentrated, and purified by FC (hexane/EtOAc 35:65) to give Int. 1D11 tert-butyl 4-[4-(dimethylcarbamoyl)phenyl]-2-methyl-piperidine-1-carboxylate (4.0 g). 5.0 g of this material was stirred ON in DCM/TFA (3.6:1, 26 mL) at 0° C. to RT, concentrated, and triturated in Et₂O/pentane to give Int. 1D10 N,N-dimethyl-4-(2-methyl-4-piperidyl)benzamide (4.0 g, TFA salt). Int. 1D10 (10.8 g) was separated into the racemic cis and trans diastereomers by SFC using a Chiral Art Cellulose SC 250×30 5 μm column operated at 30° C. and an eluent of 60% CO₂ and 40% MeOH containing 0.5% HNEt₂ (90 g/min) and a back pressure of 120 bar to give the cis racemate (first two peaks; 3.0 g) and the trans racemate (last two peaks; 7.0 g). The trans racemate (7.0 g) was resolved by SFC on a Sepiatec instrument fitted with a Chiralpak IK 250×30 5 μm column operated at 30° C. and an eluent of 70% CO₂ and 30% MeOH containing 0.5% NH₃ at a (90 g/min) and a back pressure of 100 bar to give Int. 1D1 N,N-dimethyl-4-((2S,4R)-2-methylpiperidin-4-yl)benzamide (0.95 g, first peak) and Int. 1D2 N,N-dimethyl-4-((2R,4S)-2-methylpiperidin-4-yl)benzamide (1.2 g, second peak). The absolute configuration was determined by VCD for Int. 1D1 and 1D2. The cis racemate (3.0 g) was resolved by SFC using a Chiralpak IGK 250×30 5 μm column operated at 30° C. and an eluent MeOH containing 0.5% NH₃ (30 mL/min) and a back pressure of 100 bar to give Int. 1D3 N,N-dimethyl-4-((2R,4R)-2-methylpiperidin-4-yl)benzamide (0.70 g, first peak) and Int. 1D4 N,N-dimethyl-4-((2S,4S)-2-methyl-piperidin-4-yl)benzamide (0.90 g, second peak). The absolute configuration was determined by VCD for Int. 1D3 and 1D4.

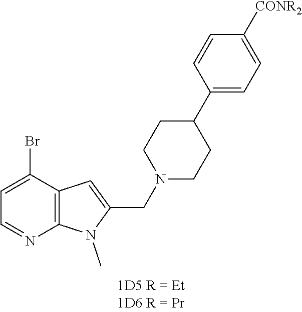

Int. 1CA4 (20 mg), HNEt₂ (24 μL), and HATU (27 mg) were stirred 0.25 h in DMF (1 mL). The OL (sat. aq. NaHCO₃/EtOAc) was washed with brine, dried, and concentrated to give Int. 1D5 4-(1-((4-Bromo-1-methyl-1H-pyrrolo[2,3-b]pyridin-2-yl)methyl)piperidin-4-yl)-N,N-diethylbenzamide (30 mg). Int. 1D6 4-(1-((4-bromo-1-methyl-1H-pyrrolo[2,3-b]pyridin-2-yl)methyl)piperidin-4-yl)-N,N-dipropyl-benzamide (37 mg, was prepared similarly from HNPr₂.

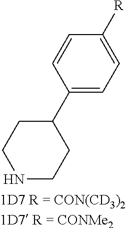

1-(1,1-Dimethylethyl) 4-(4-carboxyphenyl)-1-piperidinecarboxylate (5.1 g), HATU (13 g), and HN(CD₃)₂ (2.5 g, HCl salt) were stirred ON in DMF/DIPEA (1.8:1, 47 mL). Water was added to precipitate solid that was dissolved in DCM/MeOH (9:1), dried, concentrated, and purified by FC (hexane/EtOAc 2:3) to give tert-butyl 4-[4-[bis-(trideuteriomethyl)carbamoyl]phenyl]piperidine-1-carboxylate (4.8 g). 1.1 g of this material was stirred ON in DCM/TFA (3.6:1, 26 mL) at 0° C. to RT, concentrated, and triturated in Et₂O to give Int. 1D7 N,N-Bis(methyl-d₃)-4-(112-piperidin-4-yl)-benzamide (0.81 g, TFA salt). Int. 1D7′ N,N-dimethyl-4-(4-piperidyl)benzamide (9 g, HCl salt) was prepared similarly from 1-(1,1-dimethylethyl) 4-(4-carboxyphenyl)-1-piperidine-carboxylate (17 g) and HN(CH₃)₂ (13.6 g, HCl salt).

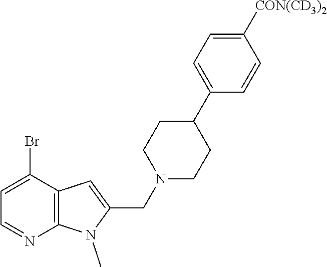

Int. 1AC4 (4.0 g), EDC (4.7 g, HCl salt), HOBt (2.5 g), and HN(CD₃)₂ (1.4 g, HCl salt) were stirred ON in DMF/DIPEA (8.2:1, 89 mL). The residue after concentration was purified by FC (EtOAc/hexane) to give Int. 1D8 4-(1-((4-bromo-1-methyl-1H-pyrrolo[2,3-b]pyridin-2-yl)methyl)piperidin-4-yl)-N,N-bis(methyl-d₃)benzamide (2.7 g).

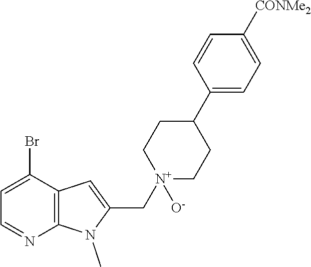

Int. 1AB1 (1 g) mCPBA (0.59 g) were stirred 2 h in DCM (10 mL) at 0° C. The OL (sat. aq. NaHCO₃/DCM) was dried and concentrated. The residue was stirred 1 h in TFA, concentrated, and purified by FC (DCM/MeOH 1:0 to 4:1) to give Int. 1D9 4-[1-[(4-bromo-1-methyl-pyrrolo[2,3-b]pyridin-2-yl)methyl]-1-oxido-piperidin-1-ium-4-yl]-N,N-dimethyl-benzamide (0.77 g).

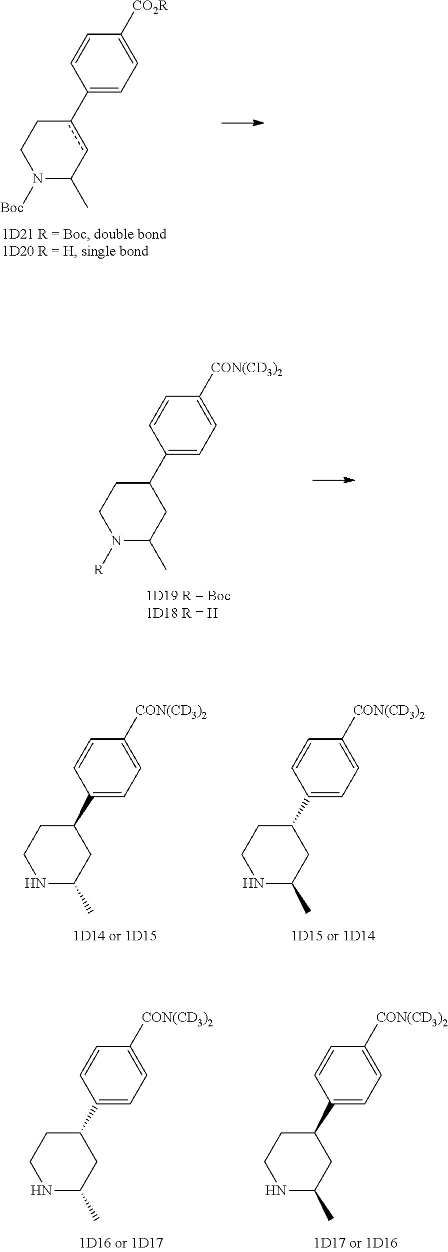

Int. 1D21 tert-butyl 4-(4-(methoxycarbonyl)phenyl)-6-methyl-3,6-dihydropyridine-1(2H)-carboxylate (5.20 g) was prepared similarly to Int. 1D12 from Int. 1D13 (10.0 g) and (4-(methoxycarbonyl)phenyl)boronic acid (7.82 g). Int. 1D20 tert-butyl 4-(4-(methoxycarbonyl)phenyl)-2-methylpiperidine-1-carboxylate (3.0 g) was prepared similarly to Int. 1D11 from Int. 1D21 (3.5 g). Int. 1D20 (3.0 g) and LiOH—H₂O (1.50 g) were stirred 4 h in THF/MeOH/water (1.3:1:1, 50 mL) at 0° C. to RT and concentrated. The OL (aq. citric acid/EtOAc) was dried and concentrated to give 4-(1-tert-butoxy-carbonyl-2-methyl-4-piperidyl)benzoic acid (1.50 g) 6.0 g of this material, HN(CD)₃)₂ (2.47 g, HCl salt), HOBt (1.90 g), and EDC-HCl (2.70 g) were stirred ON in DMF/DIPEA (1.9:1, 46 mL) at 0° C. to RT. The OL (water/EtOAc) was dried and concentrated to give Int. 1D19 tert-butyl 4-(4-(bis(methyl-d₃)carbamoyl)-phenyl)-2-methylpiperidine-1-carboxylate (4.50 g). 3.5 g of this material was stirred 4 h in DCM/TFA (2.5:1, 70 mL) at 0° C. to RT, concentrated, and triturated in Et₂O/pentane to give Int. 1D18 4-(2-methyl-4-piperidyl)-N,N-bis(trideuteriomethyl)benzamide (3.50 g, TFA salt). Int. 1D18 was separated into Ints. 1D14 N,N-bis(methyl-d₃)-4-((2S,4R)-2-methylpiperidin-4-yl)benzamide, Int. 1D15 N,N-bis-(methyl-d₃)-4-((2R,4S)-2-methylpiperidin-4-yl)benzamide, Int. 1D16 N,N-bis(methyl-d₃)-4-((2R,4R)-2-methylpiperidin-4-yl)benzamide, and Int. 1D17 N,N-bis(methyl-d₃)-4-((2S,4S)-2-methyl-piperidin-4-yl)benzamide as described for Ints. 1D1-1D4. The absolute configurations of these compounds were not determined. The absolute configurations of these compounds were determined by comparison to RT on chiral chromatography vs. Int. 1D1-1D4.

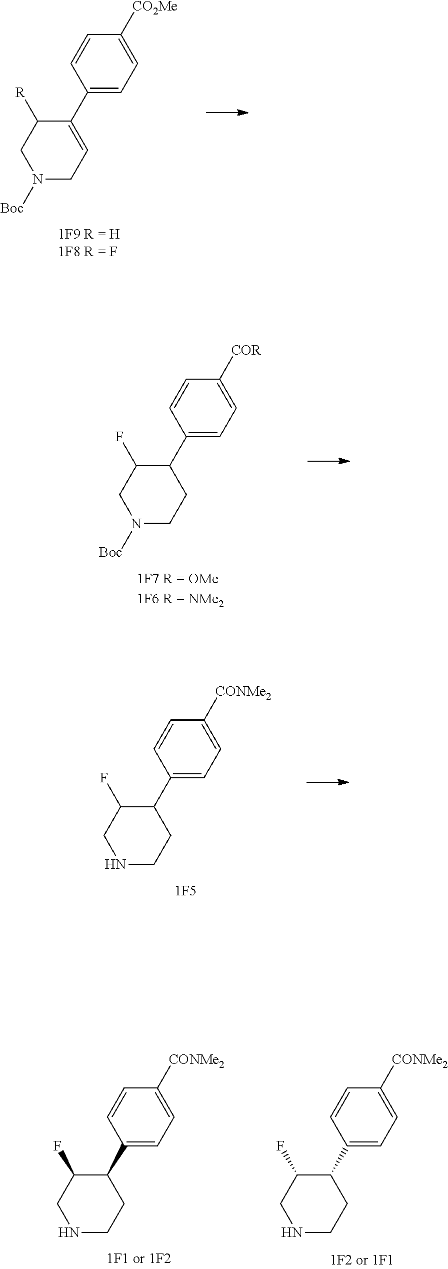

Methyl 4-bromobenzoate (25 g), PdDPPFCl₂-DCM (4.7 g), NaHCO₃ (49 g), and tert-butyl 4-(4,4,5,5-tetramethyl-1,3,2-dioxaborolan-2-yl)-3,6-dihydropyridine-1(2H)-carboxylate (39 g) were degassed in 1,4-dioxane/water (5:1, 0.6 L) and stirred ON at 110° C. The OL (water/EtOAc) was washed with brine, dried, concentrated, and purified by FC (pentane/EtOAc 99:1) to give Int. 1F9 tert-butyl 4-(4-(methoxycarbonyl)-phenyl)-3,6-dihydro-pyridine-1(2H)-carboxylate (18 g). Int. 1F9 (38 g) and SelectFluor (7.2 g) were stirred 1 h in ACN/water (3:1, 0.64 L). SelectFluor (2.1 g) was added and stirring continued 1 h. The OL (EtOAc/sat. aq. NaHCO₃) was dried, concentrated, and purified by FC (hexane/EtOAc 85:15) to give Int. 1F8 tert-butyl-3-fluoro-4-(4-methoxy-carbonylphenyl)-3,6-dihydro-2H-pyridine-1-carboxylate (6.0 g). Int. 1F8 (4.8 g) was hydrogenated ON using 10% Pd/C (0.48 g) in EtOAc (200 mL), filtered, concentrated, and purified by FC (hexane/EtOAc 9:1) to give Int. 1F7 tert-butyl-3-fluoro-4-(4-methoxycarbonylphenyl)piperidine-1-carboxylate (3.2 g). Int. 1F7 (3.5 g) and LiOH—H₂O (1.4 g) were stirred ON in MeOH/THF/H₂O (3:2:1; 25 mL) at 0° C. to RT and concentrated. The OL (10% MeOH in DCM/aq. citric acid) was dried and concentrated to give cis-4-(1-(tert-butoxycarbonyl)-3-fluoro-piperidin-4-yl)benzoic acid (2.1 g). 2.5 of this compound, HATU (4.4 g), and HN(CH₃)₂ (1.9 g, HCl salt) were stirred ON in DMF/DIPEA (2.4:1, 35 mL) at 0° C. to RT. The OL (water/5% MeOH in DCM) was dried, concentrated, and purified by FC (DCM/MeOH 95:5) to give Int. 1F6 cis-tert-butyl-4-[4-(dimethylcarbamoyl)-phenyl]-3-fluoro-piperidine-1-carboxylate (2.4 g). Int. 1F6 (1.5 g) was stirred ON in DCM/TFA (3.8:1, 13 mL) at 0° C. to RT and concentrated. The OL (aq. NaHCO₃/Et₂O and 10% MeOH in DCM) was dried and concentrated to give Int. 1F5 N,N-dimethyl-4-[cis-3-fluoro-4-piperidyl]-benzamide (1.19 g). This material was resolved by SFC using a Lux Cellulose-4 250×30 5 μm column operated at 30° C. and an eluent of 70% CO₂ and 30% 0.5% iso-propyl amine in IPA (100 g/min) and a back pressure of 100 bar to give Int. 1F1 (0.47 g) and Int. 1F2 (0.50 g) 4-((3S,4R)-3-Fluoropiperidin-4-yl)-N,N-dimethylbenzamide and 4-((3R,4S)-3-fluoropiperidin-4-yl)-N,N-dimethyl-benzamide. The absolute configurations of these compounds were not determined.

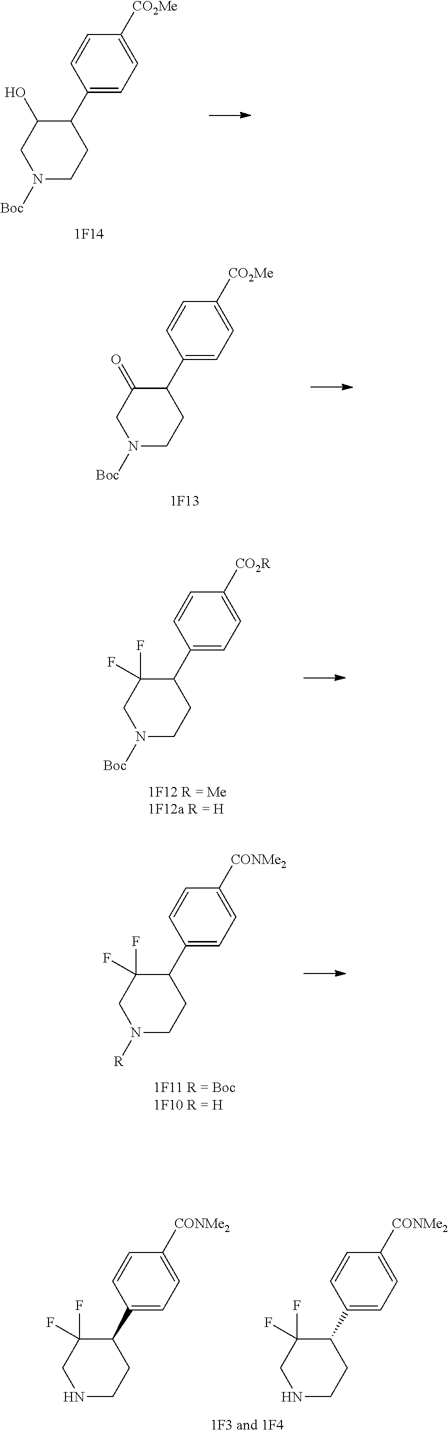

Int. 1F9 (10 g) was stirred ON in THF/2M BH₃-DMS in THF (11.6:1, 217 mL) at 0° C. to RT. 2M aq. NaOH (38 mL) and 30% aq. H₂O₂ (5.9 mL) were added and stirring was continued at 0° C. to RT over 1 h. The OL (sat. aq. NaS₂O₃/EtOAc) was washed with water and brine, dried, concentrated, and purified by FC (hexane/EtOAc 7:3) to give Int. 1F14 tert-butyl 3-hydroxy-4-(4-(methoxy-carbonyl)phenyl)piperidine-1-carboxylate (7.4 g). Int. 1F14 (8.0 g) and DMP (5.1 g) were stirred ON in DCM (200 mL) at 0° C. to RT. The OL (water/DCM) was dried, concentrated, and purified by FC (hexane/EtOAc 9:1) to give Int. 1F13 tert-butyl 4-(4-methoxy-carbonyl-phenyl)-3-oxo-piperidine-1-carboxylate (5.2 g). Int. 1F13 (5.0 g) was stirred ON in DCM/50% Deoxo-Fluor in THF (12.2:1, 108 mL) at −78° C. to RT ON. The OL (aq. NaHCO₃/DCM) was washed with sat. aq. citric acid, dried, concentrated, and purified by FC (hexane) to give Int. 1F12 tert-butyl 3,3-difluoro-4-(4-methoxycarbonyl-phenyl)piperidine-1-carboxylate (2.5 g). This material and LiOH—H₂O (1.5 g) were stirred in MeOH/THF/H₂O (3:3:1; 60 mL) at 0° C. to RT over 4 h, concentrated, and triturated in dilute aq. citric acid to give Int. 1F12a 4-(1-tert-butoxycarbonyl-3,3-difluoro-4-piperidyl)benzoic acid (2.3 g). Int. 1F12a (4.8 g), HATU (6.4 g), and HN(CH₃)₂ (2.3 g, HCl salt) were stirred ON in DMF/DIPEA (13.2:1, 108 mL) at 0° C. to RT. The OL (water/EtOAc) was dried, concentrated, and purified by FC (hexane/EtOAc 1:4) to give Int. 1F11 tert-butyl 4-(4-(dimethylcarbamoyl)-phenyl)-3,3-difluoro-piperidine-1-carboxylate (4.5 g). Int. 1F11 (4.2 g) was stirred ON in DCM/TFA (27:1, 83 mL) at 0° C. to RT and concentrated. The OL (5% MeOH in DCM/sat. aq. NaHCO₃) was dried and concentrated to give Int. 1F10 4-(3,3-difluoro-4-piperidyl)-N,N-dimethyl-benzamide (3 g). This material was resolved by SFC using a Chiralpak IG 250×30 5 μm column operated at 30° C. and an eluent of 60% CO₂ and 40% MeOH (80 g/min) and a back pressure of 60 bar to give Int. 1F3 (1.1 g) and Int. 1F4 (1.0 g) (R)-4-(3,3-Difluoropiperidin-4-yl)-N,N-dimethylbenzamide and (S)-4-(3,3-difluoropiperidin-4-yl)-N,N-dimethylbenzamide. The absolute configurations of these compounds were not determined.

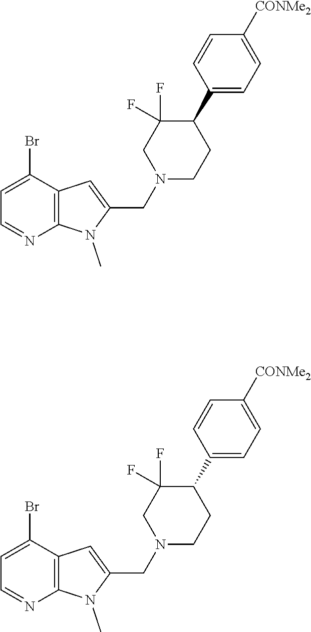

Ints. 1J1/1F3 (45 mg/60 mg), Cs₂CO₃ (169 mg), NaI (26 mg) were stirred in DMF (1 mL) for 0.5 h and HPLC-purified to give Int. 1F15 (48 mg). Int. 1F16 was prepared similarly from Int. 1F4. Ints. 1F15 and 1F16 (R)-4-(1-((4-bromo-1-methyl-1H-pyrrolo[2,3-b]pyridin-2-yl)methyl)-3,3-difluoropiperidin-4-yl)-N,N-dimethylbenzamide and (S)-4-(1-((4-bromo-1-methyl-1H-pyrrolo[2,3-b]pyridin-2-yl)methyl)-3,3-difluoropiperidin-4-yl)-N,N-dimethylbenzamide. The absolute configurations were not determined for these compounds.

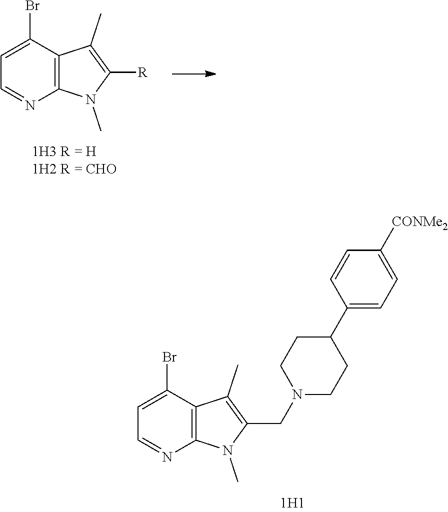

4-Bromo-3-methyl-1H-pyrrolo[2,3-b]pyridine (0.51 g) and NaH (0.12 g) were stirred 1 h in DMF (5 mL) at 0° C. before Mel (0.3 mL) was added and stirring continued for 0.3 h. The OL (water/EtOAc/Et₂O) was washed with brine, dried, concentrated to give Int. 1H3 4-bromo-1,3-dimethyl-pyrrolo[2,3-b]pyridine (0.53 g). This material was stirred 2 h in THF/1.0 M LDA in THF (2.9:1, 13.5 mL) −78° C. before DMF (1 mL) was added and stirring continued 2 h at −78° C. to RT. The OL (sat. aq. NH₄Cl/EtOAc) was washed with brine, dried, concentrated, and purified by FC (heptane/EtOAc 1:0 to 1:1) to give Int. 1H2 4-bromo-1,3-dimethyl-pyrrolo[2,3-b]pyridine-2-carbaldehyde (0.17 g). Int. 1H2 (30 mg) and N,N-dimethyl-4-(piperidin-4-yl)benzamide (42 mg) were stirred 0.5 h in DCE (2 mL). STAB (57 mg) was added and stirring continued ON. The OL (sat. aq. NaHCO₃/DCM.) was washed with brine, dried, concentrated, and purified by FC (DCM/MeOH 1:0 to 9:1) to give Int. 1H1 4-[1-[(4-bromo-1,3-dimethyl-pyrrolo[2,3-b]pyridin-2-yl)methyl]-4-piperidyl]-N,N-dimethyl-benzamide (30 mg).

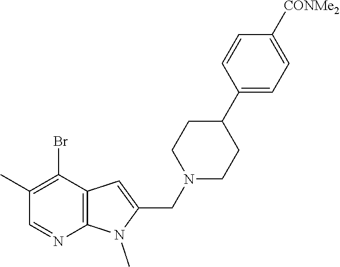

Int. 1I1 4-(1-((4-bromo-1,5-dimethyl-1H-pyrrolo[2,3-b]pyridin-2-yl)methyl)piperidin-4-yl)-N,N-dimethylbenzamide (0.16 g) as prepared similarly to Int. 1H1 from 4-bromo-5-methyl-1H-pyrrolo[2,3-b]pyridine (0.51 g) and N,N-dimethyl-4-(piperidin-4-yl)benzamide (0.18 g).

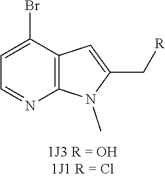

Int. 1AB8 (30 g) and NaBH₄ (19 g) were stirred 2 h in MeOH (300 mL) at 0° C. to RT. The OL (aq. Na₂S2O₃/EtOAc) was dried, concentrated, and purified by FC (pentane/EtOAc 1:1 to 2:3) to give Int. 1J3 (4-bromo-1-methyl-pyrrolo[2,3-b]pyridin-2-yl)methanol. Int. 1J3 (2.5 g) was stirred ON in DCM/Et₃N/MsCl (21.4:5.2:1, 39 mL) at 0° C. to RT and filtered. The OL layer (EtOAc/water) was washed with brine, dried, and concentrated to give Int. 1J1 4-bromo-2-(chloromethyl)-1-methyl-1H-pyrrolo[2,3-b]pyridine (2.0 g).

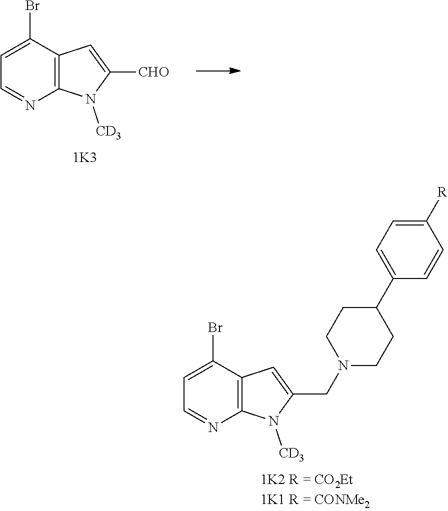

4-Bromo-1-(trideuteriomethyl)pyrrolo[2,3-b]pyridine (0.40 g) was stirred 2 h in THF/2M LDA in THF (7.1:1, 11.4 mL) at −78° C. DMF (0.7 mL) was added and stirring continued 2 h at −78° C. to RT and diluted with water to precipitate Int. 1K3 4-bromo-1-(trideuterio-methyl)pyrrolo[2,3-b]pyridine-2-carbaldehyde (0.35 g). Int. 1K3 (0.17 g) and methyl 4-(piperidin-4-yl)benzoate (0.16 g) were stirred 3 h in DCE/DIPEA (28.3:1, 15.5 mL). STAB (0.26 g) was added and stirring continued ON. The OL (water/DCM) was washed brine, dried, concentrated, and purified by FC (pentane/EtOAc 4:1) to give Int. 1K2 methyl 4-[1-[[4-bromo-1-(trideuteriomethyl)pyrrolo-[2,3-b]pyridin-2-yl]methyl]-4-piperidyl]-benzoate (0.14 g). Int. 1K2 (50 mg) was stirred 0.25 h in THF/MeOH/2M aq. NaOH (7.1:3.6:1, 3.3 mL). 10 pH was adjusted to 5 with 1M aq. HCl. The mixture was concentrated. The residue and HATU (85 mg) were dissolved in DMF/DIPEA (25.6:1, 2.1 mL) before HN(CH₃)₂ (14 mg; HCl salt) was added and stirring continued 72 h. The OL (water/DCM) was dried, concentrated, and purified by FC (DCM/MeOH 1: to 9:1) to give Int. 1K1 4-[1-[[4-bromo-1-(trideuteriomethyl)pyrrolo[2,3-b]pyridin-2-yl]-methyl]-4-piperidyl]-N,N-dimethyl-benzamide (39 mg).

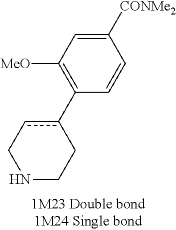

4-Bromo-3-methoxybenzoic acid (3.0 g), HN(CH₃)₂ (1.6 g, HCl salt), and HATU (5.92 g) were stirred ON in DCM/DIPEA (7.4:1, 57 mL), concentrated, and HPLC-purified to give 4-bromo-3-methoxy-N,N-dimethyl-benzamide (2.5 g). 1.0 g of this material, tert-butyl 4-(4,4,5,5-tetramethyl-1,3,2-dioxaborolan-2-yl)-3,6-dihydropyridine-1(2H)-carboxylate (1.44 g), K₂CO₃ (2.14 g), and PdDPPFCl₂-DCM (0.16 g) were degassed in dioxane/water (4:1, 10 mL) and stirred at 100° C. ON. The OL (water/EtOAc) was washed with brine, dried, concentrated, and purified by FC (hexane to EtOAc) to give tert-butyl 4-(4-(dimethylcarbamoyl)-2-methoxy-phenyl)-3,6-dihydropyridine-1(2H)-carboxylate (1.10 g). 0.6 g of this material was hydrogenated ON using 10% Pd/C (0.2 g) in MeOH (10 mL), filtered, concentrated, stirred in 4M HCl in dioxane (10 mL) ON, concentrated, and HPLC-purified to give Int. 1M24 3-methoxy-N,N-dimethyl-4-(piperidin-4-yl)benzamide (0.23 g). tert-Butyl 4-(4-(dimethylcarba-moyl)-2-methoxyphenyl)-3,6-dihydropyridine-1(2H)-carboxylate (0.50 g) was stirred ON in 4M HCl in dioxane (10 mL), concentrated, and HPLC-purified to give Int. 1M23 3-methoxy-N,N-dimethyl-4-(1,2,3,6-tetrahydropyridin-4-yl)benzamide (0.17 g).

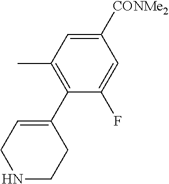

NaNO₂ (6.1 g) and 4-amino-3-fluoro-5-methyl-benzoic acid (15.0 g) were stirred 0.3 h in 48% aq. HBr (150 mL) at 0° C. CuBr (13.0 g) was added and stirring continued 4 h. The OL (aq. NaHCO₃/MTBE) was washed with water and concentrated to give 4-bromo-3-fluoro-5-methyl-benzoic acid (8.50 g). 4-Bromo-3-fluoro-5-methyl-benzoic acid (15.0 g) was stirred 5 h in DCM/SOCl₂ (83:1, 150 mL), and concentrated. The residue and HNMe₂ (3.1 g, HCl salt) were stirred 5 h DCM/Et₃N (13.6:1, 160 mL) The OL (water/DCM) was concentrated. The residue, K₂CO₃ (7.3 g), PdPDPPFCl₂-DCM (0.15 g), and tert-butyl 4-(4,4,5,5-tetramethyl-1,3,2-dioxaborolan-2-yl)-3,6-dihydro-pyridine-1(2H)-carboxylate (6.0 g) were degassed dioxane (150 mL) and stirred 48 h at 90° C. The OL (water/dioxane) was concentrated, stirred 0.5 h in THF/4M HCl in dioxane (1:1, 100 mL), concentrated, and HPLC-purified to give Int. 1M25 3-fluoro-N,N,5-trimethyl-4-(1,2,3,6-tetrahydropyridin-4-yl)benzamide (0.22 g).

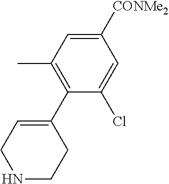

Int. 1M26 3-chloro-N,N,5-trimethyl-4-(1,2,3,6-tetrahydropyridin-4-yl)benzamide (0.11 g) was prepared similarly to Int. 1M25 from 4-bromo-3-chloro-5-methyl-benzoic acid (15 g).

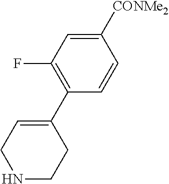

4-Bromo-3-fluoro-N,N-dimethyl-benzamide (1.0 g), tert-butyl 4-(4,4,5,5-tetramethyl-1,3,2-dioxa-borolan-2-yl)-3,6-dihydro-2H-pyridine-1-carboxylate (1.41 g), Na₂CO₃ (1.61 g), and PdDPPFCl₂-DCM (0.16 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethylcarbamo-yl)-2-fluoro-phenyl]-3,6-dihydro-2H-pyridine-1-carboxylate (1.20 g). 0.6 g of this material was stirred ON in 4M HCl in dioxane (10 mL). The mixture was concentrated and HPLC-purified to give Int. 1M29 3-fluoro-N,N-dimethyl-4-(1,2,3,6-tetrahydropyridin-4-yl)-benzamide (0.22 g).

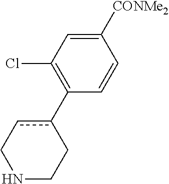

4-Bromo-3-chloro-N,N-dimethyl-benzamide (5.0 g), tert-butyl 4-(4,4,5,5-tetramethyl-1,3,2-dioxaborolan-2-yl)-3,6-dihydropyridine-1(2H)-carboxylate (0.71 g), Na₂CO₃ (8.1 g), and PdDPPFCl₂-DCM (0.78 g) were degassed in dioxane/water (4:1, 10 mL) and stirred at 100° C. ON. The OL (water/EtOAc) was dried, concentrated, and purified by FC (hexane to EtOAc) to give tert-butyl 4-[2-chloro-4-(dimethylcarbamoyl)phenyl]-3,6-dihydro-2H-pyridine-1-carboxylate (1.10 g). This material was hydrogenated ON using 10% Pt/C (1.1 g) in MeOH (10 mL), filtered, and concentrated to give tert-butyl 4-[2-chloro-4-(dimethylcarbamoyl)phenyl]-piperidine-1-carboxylate (0.60 g). This material was stirred ON in 4M HCl in dioxane (10 mL), concentrated, and HPLC-purified to give Int. 1M44 3-chloro-N,N-dimethyl-4-(piperidin-4-yl)benzamide (0.15 g). tert-Butyl 4-[2-chloro-4-(dimethyl-carbamoyl)phenyl]-3,6-dihydro-2H-pyridine-1-carboxylate (0.60 g) was stirred ON in 4M HCl in dioxane (10 mL), concentrated, and HPLC-purified to give Int. 1M43 3-chloro-N,N-dimethyl-4-(1,2,3,6-tetrahydropyridin-4-yl)benzamide (0.22 g).

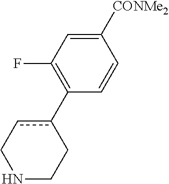

4-Bromo-3-(trifluoromethyl)benzoic acid (3.0 g), HN(CH₃)₂ (1.36 g, HCl salt) and HATU (5.1 g) stirred ON in DCM/DIPEA (8.6:1, 56 mL), concentrated, and HPLC-purified to give 4-bromo-N,N-dimethyl-3-(trifluoro-methyl)benzamide (2.40 g). 1.0 g of this material, tert-butyl 4-(4,4,5,5-tetramethyl-1,3,2-dioxaborolan-2-yl)-3,6-dihydro-pyridine-1(2H)-carboxylate (1.25 g), K₂CO₃ (1.87 g), and PdDPPFCl₂-DCM (0.14 g) were degassed in dioxane/water (4:1, 10 mL) and stirred ON at 100° C. The OL (water/EtOAc) was washed with brine, dried, concentrated, and purified by FC (hexane to EtOAc) to obtain tert-butyl 4-[4-(dimethyl-carbamoyl)-2-(trifluoromethyl)phenyl]-3,6-dihydro-2H-pyridine-1-carboxylate (1.04 g). 0.50 g of this material was stirred ON in 4M HCl in dioxane (10 mL), filtered, and HPLC-purified to give Int. 1M45 N,N-dimethyl-4-(1,2,3,6-tetrahydropyridin-4-yl)-3-(trifluoro-methyl)benzamide (0.14 g). tert-Butyl 4-[4-(dimethyl-carbamoyl)-2-(trifluoromethyl)phenyl]-3,6-dihydro-2H-pyridine-1-carboxylate (0.50 g) was hydrogenated ON using 10% Pd/C (0.5 g) in MeOH (10 mL). The mixture was filtered. The residue after concentration was stirred ON in 4M HCl in dioxane (10 mL), concentrated, and HPLC-purified to give Int. 1M46 N,N-dimethyl-4-(piperidin-4-yl)-3-(trifluoromethyl)benzamide (0.23 g).

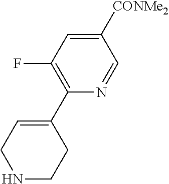

6-Chloro-5-fluoro-pyridine-3-carboxylic acid (3.0 g), HN(CH₃)₂ (1.53 g, HCl salt), and HATU (7.15 g) were stirred ON in DCM/DIPEA (5.6:1, 59 mL), concentrated, and purified by FC (hexane to EtOAc) to give 6-chloro-5-fluoro-N,N-dimethyl-pyridine-3-carboxamide (3.1 g). This material, tert-butyl 4-(4,4,5,5-tetramethyl-1,3,2-dioxa-borolan-2-yl)-3,6-dihydropyridine-1(2H)-carboxylate (5.20 g), K₃CO₃ (8.50 g), and PdDPPFCl₂-DCM (0.62 g) were degassed in dioxane/water (4:1, 50 mL) and stirred ON at 100° C. The OL (EtOAc/water) was washed with brine, dried, concentrated, and purified by FC (hexane to EtOAc) to give tert-Butyl 4-[5-(dimethylcarbamoyl)-3-fluoro-2-pyridyl]-3,6-dihydro-2H-pyridine-1-carboxylate (3.10 g). 0.13 g of this material was stirred 1.5 h in DCM/4M HCl in dioxane (3.6:1, 6.4 mL) and concentrated. The OL (DCM/aq. NaHCO₃) was dried, concentrated, and HPLC-purified to give Int. 1M47 5-fluoro-N,N-dimethyl-6-(1,2,3,6-tetrahydropyridin-4-yl)pyridine-3-carboxamide (20 mg).

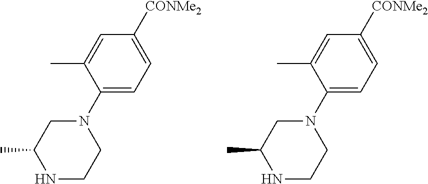

tert-Butyl (S)-2-methylpiperazine-1-carboxylate (1.0 g), ethyl 4-bromo-3-methyl-benzoate (1.46 g), Cs₂CO₃ (4.88 g), Pd₂dba₃ (0.23 g), and XPhos (0.24 g) were degassed in dioxane (20 mL) and stirred at 110° C. ON. The OL (EtOAc/water) was concentrated and purified by FC (hexane to EtOAc) to give tert-butyl (2R)-4-(4-ethoxy-carbonyl-2-methyl-phenyl)-2-methyl-piperazine-1-carboxylate (0.85 g). 0.70 g of this material and LiOH—H2O (0.22 g) were stirred 6 h in THF/water/MeOH (10 mL), concentrated, and triturated in dilute aq. citric acid to give 4-[(3R)-4-tert-butoxy-carbonyl-3-methyl-piperazin-1-yl]-3-methyl-benzoic acid (0.45 g). 4-[(3R)-4-tert-butoxycarbonyl-3-methyl-piperazin-1-yl]-3-methyl-benzoic acid (0.70 g), HNMe₂ (0.26 g, HCl salt), HOBt (0.42 g), and EDC (0.60 g, HCl salt) were stirred ON in DCM/DIPEA (10:1, 11 mL) at 0° C. to RT. The OL (water/DCM) was washed with brine, dried, concentrated, and purified by FC (hexane/EtOAc 3:2) to give tert-butyl (2R)-4-[4-(dimethylcarbamoyl)-2-methyl-phenyl]-2-methyl-piperazine-1-carboxylate (0.45 g). This material was stirred 6 h in DCM/TFA (6:1, 7 mL) at 0° C. to RT, concentrated, and triturated in Et₂O to give Int. 1M49 (R)—N,N,3-trimethyl-4-(3-methylpiperazin-1-yl)-benzamide (0.20 g). Int. 1M59 (S)—N,N,3-trimethyl-4-(3-methylpiperazin-1-yl)-benzamide was prepared similarly from tert-butyl (R)-2-methylpiperazine-1-carboxylate.

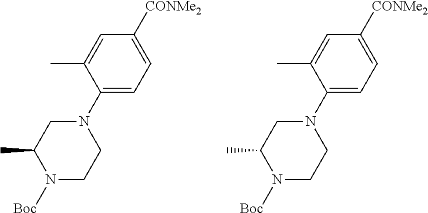

tert-Butyl (S)-2-methylpiperazine-1-carboxylate (2.5 g), NaOtBu (2.4 g), Pd₂dba₃ (0.57 g), Xphos (0.59 g), and 4-bromo-N,N-dimethylbenzamide (3.1 g) were degassed in dioxane (30 mL) and stirred ON at 100° C. The OL (Et₂O/water) was washed with brine, dried, concentrated, and purified by FC (hexane/EtOAc 1:4) to give Int. 1M50 tert-butyl (S)-4-(4-(dimethylcarbamoyl)phenyl)-2-methyl-piperazine-1-carboxylate and tert-butyl (2.4 g). Int. 1M52 (R)-4-(4-(dimethylcarbamoyl)phenyl)-2-methyl-piperazine-1-carboxylate (0.46 g) was prepared similarly from tert-butyl (R)-2-methylpiperazine-1-carboxylate (0.50 g).

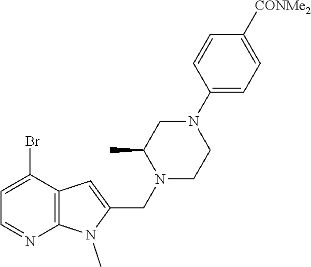

Int. 1M50 (46 mg) was stirred 1 h in DCM/TFA (1:1, 1 mL) and concentrated. The residue and 4A MS were stirred ON in DCM/DIPEA (19.5:1, 1.6 mL). Int. 1J1 (23 mg) was added and stirring was continued ON. The OL (DCM/sat. aq. NaHCO₃) was dried, concentrated, and purified by FC (DCM/MeOH 1:0 to 95:5) to give Int. 1M51 (S)-4-(4-((4-bromo-1-methyl-1H-pyrrolo[2,3-b]pyridin-2-yl)methyl)-3-methylpiperazin-1-yl)-N,N-dimethylbenzamide (40 mg).

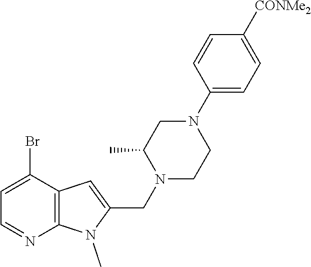

Int. 1M52 (46 mg) was stirred 1 h in DCM/TFA (1:1, 1 mL) and concentrated. The residue, Int. 1AB8 (21 mg), and 4A MS were stirred 2 h in DCM/DIPEA (19.5:1, 1.6 mL). STAB (56 mg) was added and stirring continued ON. The OL (water/DCM) was dried, concentrated, and purified by FC (DCM/MeOH 1:0 to 95:5) to give Int. 1M53 (R)-4-(4-((4-bromo-1-methyl-1H-pyrrolo[2,3-b]pyridin-2-yl)methyl)-3-methylpiperazin-1-yl)-N,N-dimethylbenzamide (30 mg).

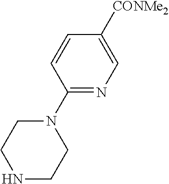

Boc-piperazine (0.40 g), 6-bromo-N,N-dimethylnicotinamide (0.59 g), NaOtBu (0.41 g), Xphos (0.10 g), and Pd₂dba₃ (0.1 g) were degassed in dioxane (4 mL) and stirred at 110° C. ON. The OL (water/EtOAc) was washed with brine, dried, concentrated, and purified by FC (pentane/EtOAc 1:1) to give 0.30 g of tert-butyl 4-(5-(dimethylcarbamoyl)pyridin-2-yl)-piperazine-1-carboxylate. 0.26 g of this material was stirred 1 h in 3M HCl in dioxane (3 mL) at 0° C. to RT and concentrated to give Int. 1M55 N,N-dimethyl-6-(piperazin-1-yl)nicotinamide (0.14 g, HCl salt).

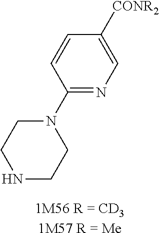

4-(4-tert-Butoxycarbonylpiperazin-1-yl)benzoic acid (1.0 g), HN(CD₃)₂ (0.43 g, HCl salt), HOBt (0.66 g), and EDC (0.94 g, HCl salt) were stirred ON in DCM/DIPEA (15.4:1, 21 mL). The OL (water/DCM) was washed with brine, dried, concentrated, and purified by FC (pentane/EtOAc 1:1) to give tert-butyl 4-(5-(bis(methyl-d₃)carbamoyl)-pyridin-2-yl)piperazine-1-carboxylate (0.95 g). This material was stirred in DCM/TFA (6.7:1, 35 mL), concentrated, and triturated in Et₂O to give Int. 1M56 N,N-bis(methyl-d₃)-6-(piperazin-1-yl)nicotinamide (0.85 g). Int. 1AE20 (14 g) was hydrogenated ON using 10% Pd/C (3.5 g) in MeOH (200 mL), filtered, and concentrated to give tert-butyl 4-[5-(dimethyl-carbamoyl)-2-pyridyl]piperidine-1-carboxylate (11 g). 0.9 g of this material was stirred in DCM/TFA (10:1, 11 mL) at 0° C. to RT, concentrated, and triturated in Et₂O to give Int. 1M57 N,N-dimethyl-6-(piperidin-4-yl)nicotinamide (0.50 g, TFA salt).

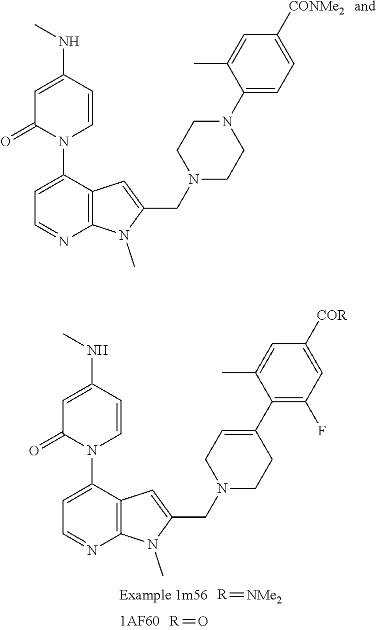

Ints. 1M1/1S7 (15 mg/22 mg, HCl salt) were stirred 0.5 h in DCE (0.5 mL). STAB (25 mg) was added and stirring was continued ON. The mixture was filtered. The filtrate was stirred 0.5 h in TFA (0.5 mL), concentrated, and purified by HPLC to give Example 1m6 N,N,3-Trimethyl-4-(4-((1-methyl-4-(4-(methylamino)-2-oxopyridin-1(2H)-yl)-1H-pyrrolo[2,3-b]pyridin-2-yl)methyl)piperazin-1-yl)benzamide (6.6 mM in DMSO (0.70 mL). Example 1m56 3-Fluoro-N,N,5-trimethyl-4-(1-((1-methyl-4-(4-(methylamino)-2-oxopyridin-1(2H)-yl)-1H-pyrrolo[2,3-b]pyridin-2-yl)methyl)-1,2,3,6-tetrahydropyridin-4-yl)benzamide (10 mM in DMSO (1.2 mL)) was prepared similarly to Example 1m6 from Int. 1M25 (17 mg). Example 1m56 (2.4 g) and LiOH—H₂O (1.1 g) were stirred 3 h in THF/water (2.5:1, 28 mL) at 0° C. to 60° C. The residue after concentration was triturated in aq. HCl to give Int. 1M60 3-fluoro-5-methyl-4-(1-((1-methyl-4-(4-(methylamino)-2-oxopyridin-1(2H)-yl)-1H-pyrrolo[2,3-b]pyridin-2-yl)methyl)-1,2,3,6-tetrahydropyridin-4-yl)benzoic acid (2.4 g).

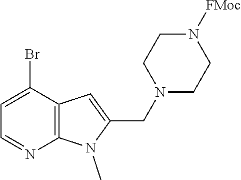

Int. 1AB8 (0.25 g) and 9H-fluoren-9-ylmethyl piperazine-1-carboxylate (0.48 g) were stirred 2 h in DCE/AcOH (100:1, 10 mL). STAB (0.44 g) was added and stirring continued 2 h at 0° C. to RT. The OL (sat. aq. NaHCO₃/DCM) was washed with brine, dried, and concentrated to give Int. 1N2 9H-fluoren-9-ylmethyl 4-[(4-bromo-1-methyl-pyrrolo[2,3-b]pyridin-2-yl)methyl]piperazine-1-carboxylate (0.20 g).

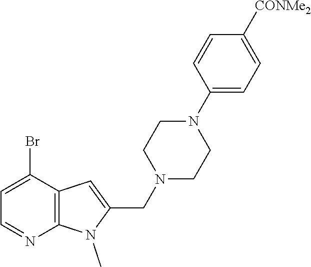

Int. 1AB8 (0.35 g) and N,N-dimethyl-4-(piperazin-1-yl)benzamide (0.40 g, HCl salt) were stirred 4 h in DCE/DIPEA (6.5:1, 11.6 mL). STAB (0.94 g) was added and stirring continued ON. The OL (water/DCM) was washed with brine, dried, concentrated, and purified by FC (hexane/EtOAc 1:4) to give Int. 1N4 4-(4-((4-bromo-1-methyl-1H-pyrrolo[2,3-b]pyridin-2-yl)methyl)piperazin-1-yl)-N,N-dimethyl-benzamide (0.44 g).

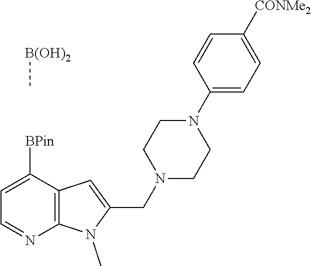

Int. 1N4 (25 mg), KOAc (11 mg), PdDPPFCl₂-DCM (4 mg), and B₂Pin₂ (15 mg) were degassed in dioxane (3 mL) and stirred 4 h at 70° Ch. KOAc (11 mg), and B₂Pin₂ (15 mg), and PdDPPFCl₂-DCM (4 mg) were added and stirring continued 4 h at 70° C. and ON at RT. KOAc (11 mg), B₂Pin₂ (15 mg), and PdDPPFCl₂-DCM (4 mg) were added and stirring continued 6 h at 90° C. The mixture was filtered and HPLC-purified to give Int. 1N5 N,N-dimethyl-4-[4-[[1-methyl-4-(4,4,5,5-tetramethyl-1,3,2-dioxa-borolan-2-yl)pyrrolo[2,3-b]pyridin-2-yl]methyl]piperazin-1-yl]benzamide and (2-((4-(4-(dimethyl-carbamoyl)-phenyl)piperazin-1-yl)methyl)-1-methyl-1H-pyrrolo[2,3-b]pyridin-4-yl)boronic acid.

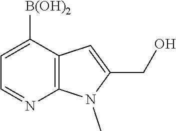

Int. 1J3 (1.0 g), B₂Pin₂ (1.58 g), Pd(PPh₃)₂Cl₂ (0.29 g), and KOAc (1.02 g) were degassed dioxane (10 mL) and stirred 3 h at 100° C. and concentrated. The residue decanted with pentane and concentrated to give Int. 103 [2-(hydroxymethyl)-1-methyl-pyrrolo[2,3-b]pyridin-4-yl]boronic acid (0.8 g).

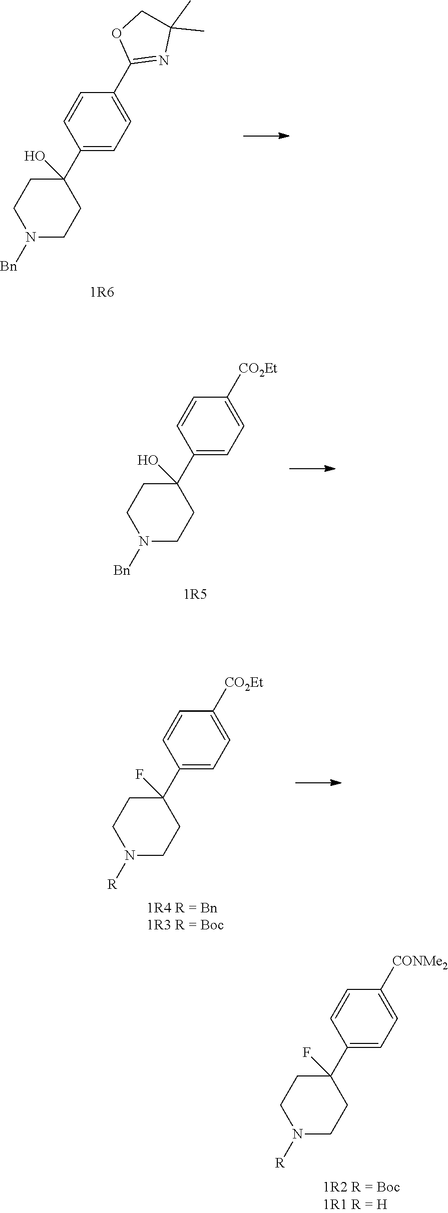

2-(4-Bromophenyl)-4,4-dimethyl-4,5-dihydrooxazole (10 g) and Mg turnings (1.1 g) and I₂ (50 mg) were refluxed 1 h in THF (50 mL). 1-Benzylpiperidin-4-one (8.0 mL) was added before refluxing 3 h. The OL (sat. aq. NH₄Cl/MTBE) was washed with water, brine, dried, and concentrated to give Int. 1R6 1-benzyl-4-(4-(4,4-dimethyl-4,5-dihydro-oxazol-2-yl)phenyl)piperidin-4-ol. Int. 1R6 (7.0 g) was stirred ON in EtOH/96% H₂SO₄ (10:1, 400 mL) at 90° C. and concentrated. The OL (sat. aq. NaHCO₃/DCM) was dried, concentrated, and purified by FC (DCM/2M NH₃ in MeOH 1:0 to 4:1) to give Int. 1R5 ethyl 4-(1-benzyl-4-hydroxypiperidin-4-yl)-benzoate. Int. 1R5 (0.06 g) was dissolved in DCM (10 mL) at −78° C. DAST (0.28 mL) was added and stirring continued 2 h at −78° C. to RT over 2 h. The OL (DCM/water) was washed with brine, dried, concentrated, and HPLC-purified to give Int. 1R4 ethyl 4-(1-benzyl-4-fluoro-piperidin-4-yl)benzoate. Int. 1R4 (2.0 g) was stirred 3 h in DCM (40 mL) and 1-chloroethyl carbono-chloridate (0.76 mL) at 0° C. to RT and concentrated. The residue was refluxed 0.5 h in MeOH (20 mL) and concentrated. The residue and Boc₂O (1.56 g) were stirred ON in DCM/Et₃N (14.7:1, 27 mL). The OL (EtOAc/water) was washed with brine, dried, concentrated, and purified by FC (heptane/EtOAc 1:0 to 1:1) to give 4-(1-(tert-butoxycarbonyl)-4-fluoropiperidin-4-yl)benzoic acid. 0.77 g of this material, HATU (1.18 g), and HN(CH₃)₂ (0.29 g, HCl salt) were stirred 1 h in DMF/DIPEA (5:1, 10 mL). The OL (EtOAc/water) was dried, concentrated, and purified by FC (heptane to EtOAc/MeOH 95:5) to give Int. 1R2 tert-butyl 4-(4-(dimethylcarbamoyl)-phenyl)-4-fluoropiperidine-1-carboxylate. Int. 1R2 (0.55 g) was stirred 1 h in MeOH/4M HCl in dioxane (1:1, 20 mL), concentrated, and purified by SCX and HPLC to give Int. 1R1 4-(4-fluoropiperidin-4-yl)-N,N-dimethylbenzamide (62 mg).

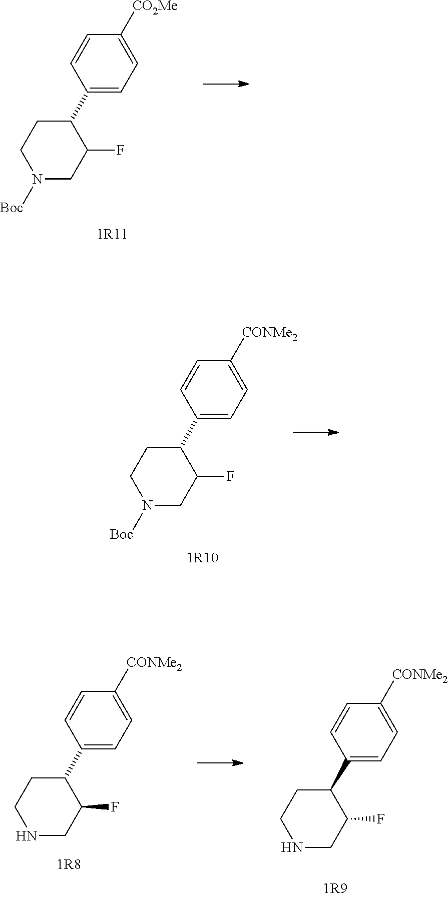

Int. 1AE14 (10.0 g) was stirred ON in THF/2.0 M BH₃-Me₂S in THF (11:6:1, 218 mL) at 0° C. to RT. 2.0 M aq. NaOH (38 mL) and 30% aq. H₂O₂ (5.9 mL) were added and stirring continued 1 h. The OL (sat. aq. Na₂S2O₃/EtOAc) was washed with water and brine, dried, concentrated, and purified by FC (hexane/EtOAc 7:3) to give tert-butyl 3-hydroxy-4-(4-(methoxycarbonyl)-phenyl)piperidine-1-carboxylate (7.4 g). 7.0 g of this material was dissolved in DCM (140 mL) at −78° C. 50% Deoxo-Fluor in THF (4.5 mL) was added and stirring continued 6 h at −78° C. to RT. The OL (sat. aq. NaHCO₃/DCM) were washed with sat. aq. citric acid, dried, concentrated, and purified by FC (hexane/EtOAc 85:15) to give Int. 1R11 tert-butyl 3-fluoro-4-(4-(methoxycarbonyl)phenyl)-piperidine-1-carboxylate (4.8 g). Int. 1R11 (10.0 g) and LiOH—H₂O (6.1 g) were stirred ON in MeOH/THF/water (2:4:1, 230 mL) at 0° C. to RT and concentrated. The OL (aq. citric acid/10% MeOH in DCM) was dried and concentrated to give 4-(1-(tert-butoxy-carbonyl)-3-fluoropiperidin-4-yl)benzoic acid (8.0 g). 22 g of this material, HATU (39 g), and HN(CH₃)₂ (16.5 g, HCl salt) were stirred ON in DMF/DIPEA (6:7:1, 0.46 L) at 0° C. to RT. The OL (water/MeOH/DCM (5:95)) was dried, concentrated, and purified by FC (hexane/EtOAc 2:3) to give tert-butyl 4-(4-(dimethylcarbamoyl)phenyl)-3-fluoropiperidine-1-carboxylate (20 g). This material was stirred ON in DCM/TFA (37:1, 513 mL) at 0° C. to RT ON and concentrated. The aq. layer (water/Et₂O) was basified with sat. aq. NaHCO₃ and extracted with MeOH/DCM (5:95). The OLs were dried and concentrated to give 4-(3-fluoropiperidin-4-yl)-N,N-dimethyl-benzamide (14.5 g). This material was resolved by SFC using a Chiralpak IG 250×25 5 μm column operated at 30° C. using an eluent of 70% CO₂ and 30% MeOH containing 0.5% HNEt₂ (100 g/min) and a back pressure of 100 bar to give Int. 1R8 (5.3 g, first peak) and Int. 1R9 (5.1 g, second peak) 4-((3R,4R)-3-fluoropiperidin-4-yl)-N,N-dimethylbenzamide and 4-((3S,4S)-3-fluoropiperidin-4-yl)-N,N-dimethylbenzamide. The absolute configurations of these compounds were not determined.

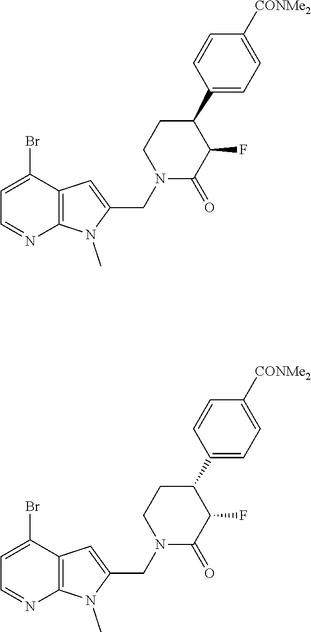

Int. 1F1 (0.10 g), DIPEA (0.21 mL), KI (20 mg), and Int. 1J1 were stirred ON in DMF (5 mL). The OL (EtOAc/water) was dried, concentrated, and purified by FC (hexane/EtOAc 3:7) to give Int. 1R12 N,N-Dimethyl-4-[(3R,4S)-1-[(4-bromo-1-methyl-pyrrolo[2,3-b]-pyridin-2-yl)methyl]-3-fluoro-4-piperidyl]benzamide (0.12 g). Int. 1R13 N,N-dimethyl-4-[(3S,4R)-1-[(4-bromo-1-methyl-pyrrolo[2,3-b]pyridin-2-yl)methyl]-3-fluoro-4-piperidyl]benzamide (0.12 g) was prepared similarly from Ints. 1F2/1J1 (0.10 g/0.16 g).

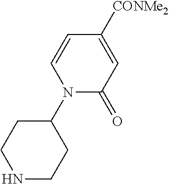

KOtBu (74 mg), K₂CO₃ (0.17 g), N,N-dimethyl-2-oxo-1H-pyridine-4-carboxamide (0.10 g), and tert-butyl 4-(p-tolylsulfonyloxy)piperidine-1-carboxylate (0.24 g) were stirred ON in DME (5 mL) at 110° C. ON. The OL (EtOAc/water) was dried, concentrated, and purified by FC (hexane to EtOAc) to give tert-butyl 4-[4-(dimethyl-carbamoyl)-2-oxo-1-pyridyl]piperidine-1-carboxylate (70 mg). tert-Butyl 4-[4-(dimethylcarbamoyl)-2-oxo-1-pyridyl]piperidine-1-carboxylate (0.20 g) was stirred in DCM/TFA (10:1, 5.5 mL) and concentrated to give Int. 1S2 N,N-Dimethyl-2-oxo-1-(4-piperidyl)pyridine-4-carbox-amide and Int. 1S2′ N,N-dimethyl-2-(4-piperidyloxy)pyridine-4-carboxamide (0.15 g, TFA salt).

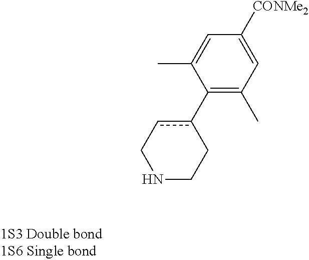

4-Bromo-N,N,3,5-tetramethylbenzamide (5.5 g), tert-butyl 4-(4,4,5,5-tetramethyl-1,3,2-dioxaborolan-2-yl)-3,6-dihydropyridine-1(2H)-carboxylate (7.3 g), PdDPPFCl₂-DCM (0.88 g), and NaHCO₃ (5.4 g) were degassed in dioxane/water (3:1, 47 mL) and stirred ON at 100° C. The OL (EtOAc/water) was dried, concentrated, and purified by FC (pentane/EtOAc 1:1) to give tert-butyl 4-[4-(dimethylcarbamoyl)-2,6-dimethyl-phenyl]-3,6-dihydro-2H-pyridine-1-carboxylate (4.5 g). 0.3 g of this material was stirred ON in 2.9M HCl in dioxane (7 mL) at 0° C. to R, concentrated, and triturated in Et₂O. The solid was treated with sat. aq. NaHCO₃ and concentrated. The residue was dissolved in DCM, dried, and concentrated to give Int. 1S3 N,N,3,5-tetramethyl-4-(1,2,3,6-tetrahydro-pyridin-4-yl)benzamide (0.15 g). tert-Butyl 4-[4-(dimethylcarbamoyl)-2,6-dimethyl-phenyl]-3,6-dihydro-2H-pyridine-1-carboxylate (50 mg) was hydrogenated 48 h using PtO₂ (15 mg) in AcOH (3 mL), filtered, and concentrated to give Int. 1S6 N,N,3,5-tetramethyl-4-(piperidin-4-yl)benzamide (35 mg).

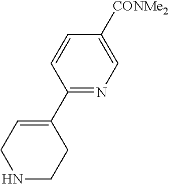

6-Bromo-N,N-dimethylnicotinamide 20.0 g), tert-butyl 4-(4,4,5,5-tetramethyl-1,3,2-dioxaborolan-2-yl)-3,6-dihydropyridine-1(2H)-carboxylate (27.0 g), NaHCO₃ (25.7 g), and PdDPPFCl₂-DCM (0.71 g) were degassed in dioxane/water (4:1, 0.5 L), refluxed ON, and filtered. The OL (water/EtOAc) was dried, concentrated, and purified by FC (pentane/EtOAc 45:55) to give tert-butyl 4-[5-(dimethyl-carbamoyl)-2-pyridyl]-3,6-dihydro-2H-pyridine-1-carboxylate (17 g). 1.0 g of this material was stirred ON in DCM/4M HCl in dioxane (2.6:1, 14 mL) at 0° C. to RT. The mixture was combined with another two batches prepared on 0.1 g scale and concentrated. The residue was stirred in water at pH 8 (adjusted with NaHCO₃). The mixture was purified by FC (reverse-phase C₁₈ column; 8% ACN in 0.001% aq. formic acid) to give Int. 1S4 N,N-dimethyl-1′,2′,3′,6′-tetrahydro-[2,4′-bipyridine]-5-carboxamide (0.60 g).

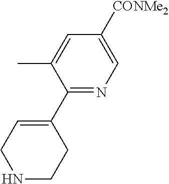

6-Bromo-N,N,5-trimethyl-pyridine-3-carboxamide (3.2 g), NaHCO₃ (3.9 g), PdDPPFCl₂-DCM (0.27 g), and tert-butyl 4-(4,4,5,5-tetramethyl-1,3,2-dioxaborolan-2-yl)-3,6-dihydropyridine-1(2H)-carboxylate (6.1 g) were degassed in dioxane/water (4:1, 13 mL) and stirred ON at 110° C. The mixture was filtered, concentrated, and purified by FC (hexane/EtOAc 1:4) to give tert-butyl 4-[5-(dimethyl-carba-moyl)-3-methyl-2-pyridyl]-3,6-dihydro-2H-pyridine-1-carboxylate (4.0 g). 0.35 g of this material was stirred ON in DCM/TFA (10:1, 9 mL) at 0° C. to RT, concentrated, and triturated in Et₂O/pentane to give Int. 1S5 N,N,3-trimethyl-1′,2′,3′,6′-tetrahydro-[2,4′-bipyridine]-5-carboxamide (0.35 g, TFA salt).

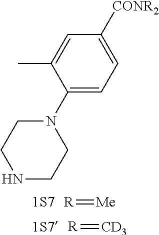

tert-Butyl piperazine-1-carboxylate (5.0 g), 4-bromo-N,N,3-trimethylbenzamide (7.1 g), Pd₂dba₃ (1.19 g), XPhos (1.29 g), and NaOtBu (5.19 g) were degassed in dioxane (50 mL) and stirred ON at 100° C. and filtered. The OL (water/EtOAc) was dried, concentrated, and purified by FC (pentane/EtOAc 3:7) to give tert-butyl 4-[4-(dimethyl-carbamoyl)-2-methyl-phenyl]piperazine-1-carboxylate (6.1 g). 7.2 g of this material was stirred ON in 4M HCl in dioxane/DCM (1.8:1, 55 mL) at 0° C. to RT, concentrated, and triturated in Et₂O/pentane to give Int. 1S7 N,N,3-trimethyl-4-(piperazin-1-yl)benzamide (5.0 g, HCl salt). Int. 1S7′ 3-methyl-N,N-bis(methyl-d₃)-4-(piperazin-1-yl)benzamide was prepared similarly from 4-bromo-3-methyl-N,N-bis(methyl-d₃)benzamide.

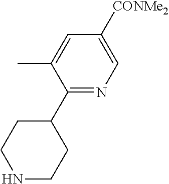

6-Bromo-N,N,5-trimethyl-pyridine-3-carboxamide (3.2 g), NaHCO₃ (3.9 g), and PdDPPFCl₂-DCM (0.27 g), tert-butyl 4-(4,4,5,5-tetramethyl-1,3,2-dioxaborolan-2-yl)-3,6-dihydropyridine-1(2H)-carboxylate (6.1 g) were degassed in dioxane/water (4:1, 14 mL) and stirred ON at 110° C. The mixture was filtered, concentrated, and purified by FC (hexane/EtOAc 1:4) to give tert-butyl 4-[5-(dimethylcarba-moyl)-3-methyl-2-pyridyl]-3,6-dihydro-2H-pyridine-1-carboxylate (4.0 g). 4.2 g of this material was hydrogenated ON using 10% Pd/C (2.0 g) in MeOH (100 mL), filtered, concentrated, and purified by FC (hexane/EtOAc 1:9) to give tert-butyl 4-[5-(dimethyl-carbamoyl)-3-methyl-2-pyridyl]piperidine-1-carboxylate (3.5 g). 4.3 g of this material was stirred ON in DCM/TFA (1.5:1, 25 mL) at 0° C. to RT, concentrated, and triturated in pentane/Et₂O to give Int. 1S8 N,N,5-Trimethyl-6-(piperidin-4-yl)nicotinamide (5.0 g, TFA salt).

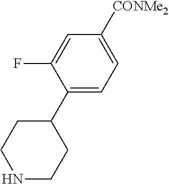

4-Bromo-3-fluoro-N,N-dimethyl-benzamide (1.0 g), tert-butyl 4-(4,4,5,5-tetramethyl-1,3,2-dioxaborolan-2-yl)-3,6-dihydropyridine-1(2H)-carboxylate (1.51 g), Na₂CO₃ (1.72 g), and PdDPPFCl₂-DCM (0.17 g) were degassed in dioxane/water (4:1, 10 mL) and stirred ON at 100° C. The OL (water/EtOAc) was washed with brine, dried, concentrated, and purified by FC (hexane to EtOAc) to give tert-butyl 4-[4-(dimethyl-carbamoyl)-2-fluoro-phenyl]-3,6-dihydro-2H-pyridine-1-carboxylate (1.20 g). This material was hydrogenated ON using 10% Pd/C (45 mg) in MeOH (2 mL), filtered, concentrated, stirred ON in 4M HCl in dioxane (10 mL), concentrated, and HPLC-purified to give Int. 1S18 3-fluoro-N,N-dimethyl-4-(piperidin-4-yl)benzamide (63 mg).

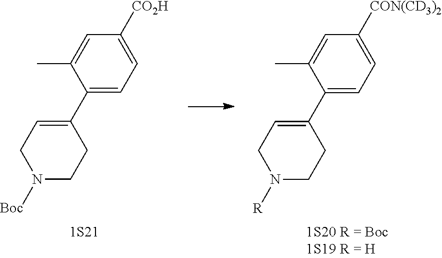

96% aq. H₂SO₄ (2.5 mL) was added to a solution of 4-bromo-3-methyl-benzoic acid (20 g) in MeOH (300 mL). The mixture was stirred at 90° C. ON and concentrated. The organic layer (sat. aq. NaHCO₃/EtOAc) was dried and concentrated to give methyl 4-bromo-3-methyl-benzoate (20 g). 10 g of this material, tert-butyl 4-(4,4,5,5-tetramethyl-1,3,2-dioxaborolan-2-yl)-3,6-dihydropyridine-1(2H)-carboxylate (14.8 g), Na₂CO₃ (13.8 g), and PdDPPFCl₂-DCM (0.84 g) were degassed in dioxane/water (2.3:1, 100 mL), stirred ON at 90° C., filtered, concentrated, and purified by FC (hexane/EtOAc 95:5) to give tert-butyl 4-(4-methoxycarbonyl-2-methyl-phenyl)-3,6-dihydro-2H-pyridine-1-carboxylate (7.0 g). 2.5 g of this material and LiOH—H₂O (2.0 g) were stirred ON in EtOH/THF/water (2:1:1, 24 mL) at 0° C. to RT, concentrated, and triturated in dilute aq. citric acid to give Int. 1S21 4-(1-tert-butoxycarbonyl-3,6-dihydro-2H-pyridin-4-yl)-3-methyl-benzoic acid (2.0 g). Int. 1S20 (0.70 g) was prepared similarly to Int. 1D8 from Int. 1S21 (2.0 g). Int. 1S19 3-methyl-N,N-bis(methyl-d₃)-4-(1,2,3,6-tetrahydropyridin-4-yl)benzamide (0.31 g) was prepared similarly to Int. 1S7 from Int. 1S20 (0.80 g).

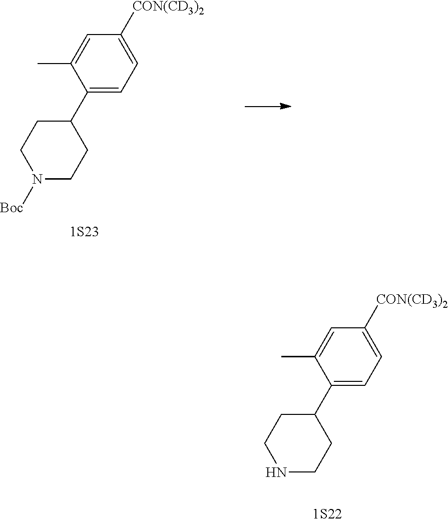

Int. 1S20 (0.35 g) was hydrogenated ON using 10% Pd/C (50 mg) in MeOH (10 mL), filtered, and concentrated to give Int. 1S23 (0.21 g). Int. 1S22 3-methyl-N,N-bis-(methyl-d₃)-4-(piperidin-4-yl)benzamide (0.45 g) was prepared similarly to Int. 1S7 from Int. 1S23 (0.90 g).

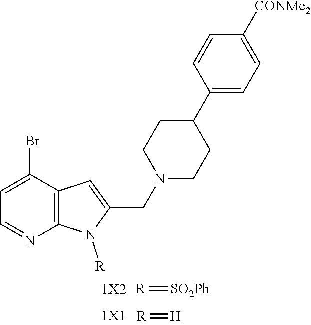

Int. 1A6 (0.30 g), N,N-dimethyl-4-(4-piperidyl)benzamide (0.34 g), and 4A MS were stirred 0.3 h in DCM (5 mL). STAB (0.52 g) was added and stirring was continued ON. The mixture was filtered, concentrated, and purified by FC (heptane to EtOAc/MeOH 1:4) to give Int. 1X2 4-[1-[[1-(benzenesulfonyl)-4-bromo-pyrrolo-[2,3-b]-pyridin-2-yl]-methyl]-4-piperidyl]-N,N-dimethyl-benzamide (0.22 g). Int. 1X2 (0.12 g) was stirred 2.5 h in EtOH/4M aq. NaOH (2.5:1, 2.8 mL) at 80° C. and concentrated. The OL (water/DCM) was concentrated to give Int. 1X1 4-[1-[(4-bromo-1H-pyrrolo[2,3-b]pyridin-2-yl)methyl]-4-piperidyl]-N,N-dimethyl-benzamide (94 mg).

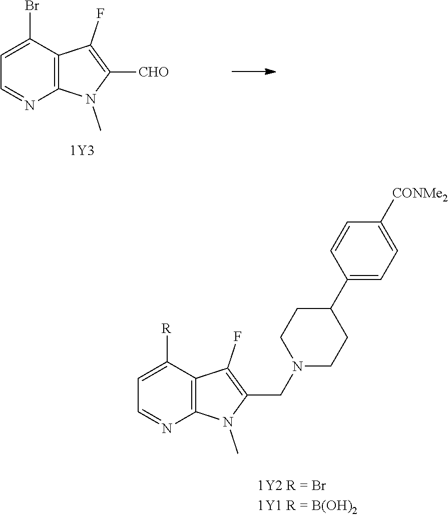

4-Bromo-3-fluoro-1-methyl-pyrrolo[2,3-b]pyridine (1.9 g) was stirred 2 h in THF/2.0 M LDA in THF (5:1, 24 mL) at −78° C. DMF (1.6 mL in THF (5 mL)) was added and stirring continued 2 h at −78° C. to RT. The OL (water/DCM) was washed with brine, dried, concentrated, and purified by FC (pentane/EtOAc 1:4) to give Int. 1Y3 4-bromo-3-fluoro-1-methyl-pyrrolo[2,3-b]pyridine-2-carbaldehyde (0.4 g). Int. 1Y3 (0.39 g) and N,N-dimethyl-4-(4-piperidyl)benzamide trifluoro acetate (0.38 g) were stirred ON in DCE/DIPEA (3.8:1, 6.3 mL) at 0° C. to RT. STAB (0.64 g) was added and stirring continued ON at 0° C. to RT. The OL (EtOAc/water) was washed brine, dried, concentrated, and purified by FC (pentane to EtOAc) to give Int. 1Y2 4-[1-[(4-bromo-3-fluoro-1-methyl-pyrrolo[2,3-b]pyridin-2-yl)methyl]-4-piperidyl]-N,N-dimethyl-benzamide (0.38 g). Int. 1Y2 (0.25 g), B₂Pin₂ (0.17 g), KOAc (0.16 g), and PdDPPFCl₂-DCM (37 mg) were degassed in THF (20 mL) and ON at 100° C., concentrated, and triturated in pentane to give Int. 1Y1 (2-((4-(4-(dimethylcarbamoyl)phenyl)-piperidin-1-yl)methyl)-3-fluoro-1-methyl-1H-pyrrolo[2,3-b]pyridin-4-yl)boronic acid (0.2 g).

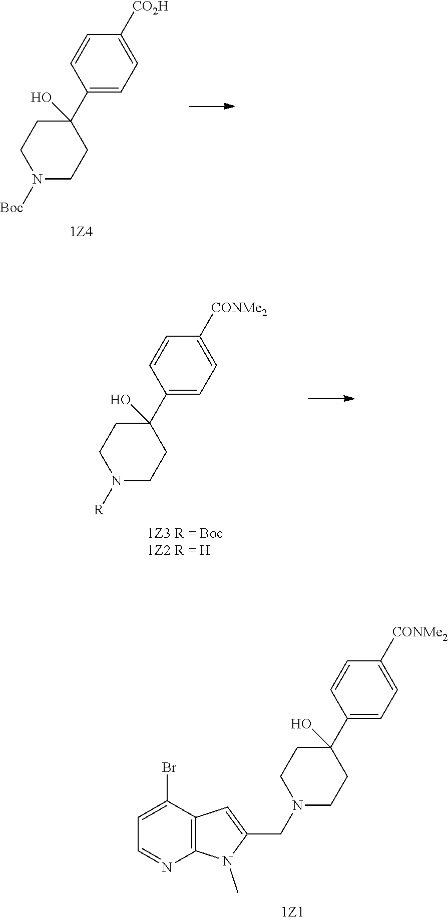

Int. 1R5 (0.5 g) and LiOH (181 mg) were stirred ON in MeOH/THF/water (3:5:1, 9 mL) and concentrated to give Int. 1Z4 4-(1-benzyl-4-hydroxypiperidin-4-yl)benzoic acid (0.40 g). Int. 1Z4 (0.40 g), HN(CH₃)₂ (0.31 g, HCl salt), and HATU (636 mg) were stirred ON in DMF/DIPEA (7:3:1, 9.1 mL). The OL (EtOAc/water) was concentrated, and purified by FC (heptane/EtOAc 5:3 to 3:7) to give Int. 1Z3 4-(1-benzyl-4-hydroxy-piperidin-4-yl)-N,N-dimethyl-benzamide. Int. 1Z3 (1.0 g) was hydrogenated 32 h using 10% Pd/C (0.5 g) in MeOH (20 mL), filtered, and concentrated to give Int. 1Z2 4-(4-hydroxypiperidin-4-yl)-N,N-dimethylbenzamide. Int. 1Z2 (0.20 g) and 4-bromo-1H-pyrrolo[2,3-b]pyridine-2-carboxaldehyde (0.19 g) were stirred 4 h in DCE/DIPEA (10:1, 3.3 mL). STAB (0.51 g) was added and stirring continued 32 h. The OL (water/DCM) was concentrated and purified by FC (hexane/EtOAc 1:0 to 4:1) to give Int. 1Z1 4-[1-[(4-Bromo-1-methyl-pyrrolo[2,3-b]pyridin-2-yl)methyl]-4-hydroxy-4-piperidyl]-N,N-dimethyl-benzamide (0.15 g).

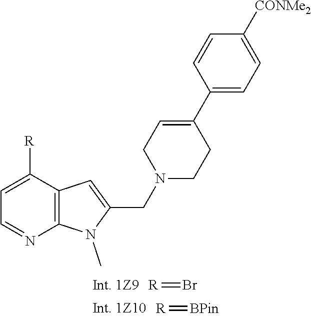

Int. 1J1 (0.97 g), KI (12 mg), Int. 1AE7 (1 g), and K₂CO₃ (1.6 g) were stirred 11 h in DMF (10 mL). The OL (water/MTBE) was concentrated to give Int. 1Z9 4-(1-((4-bromo-1-methyl-1H-pyrrolo[2,3-b]pyridin-2-yl)methyl)-1,2,3,6-tetrahydropyridin-4-yl)-N,N-dimethylbenzamide (0.67 g). Int. 1Z9 (0.67 g), KOAc (0.44 g), PdDPPFCl₂-DCM (24 mg), and bis(pinacolato)diboron (0.75 g) were stirred 48 h in dioxane (10 mL) at 90° C. under an inert atmosphere. The OL (water/MTBE) was concentrated to give Int. 1Z10 N,N-dimethyl-4-(1-((1-methyl-4-(4,4,5,5-tetramethyl-1,3,2-dioxaborolan-2-yl)-1H-pyrrolo-[2,3-b]pyridin-2-yl)methyl)-1,2,3,6-tetrahydropyridin-4-yl)benzamide (0.72 g). 6-bromopyridazin-3-amine (0.25 g) and di-tert-butyl dicarbonate (0.38 g) were stirred ON in 1M LiHMDs in THF (1.9 mL) and THF (5 mL) at 0° C. to RT under an inert atmosphere. The OL (sat. aq. NH₄Cl/EtOAc) was dried, and concentrated to give Int. 1Z11 tert-butyl (6-bromopyridazin-3-yl)carbamate (0.15 g).

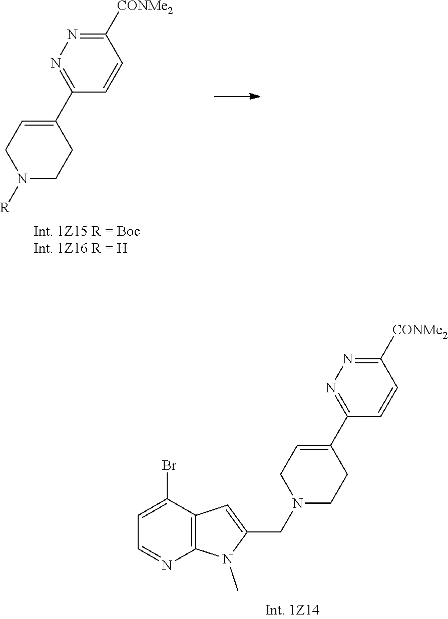

6-Chloro-N,N-dimethylpyridazine-3-carboxamide (0.85 g), tert-butyl 4-(4,4,5,5-tetramethyl-1,3,2-dioxaborolan-2-yl)-3,6-dihydropyridine-1(2H)-carboxylate (1.6 g), K₂CO₃ (2.5 g), and PdDPPFCl₂-DCM (0.19 g) were stirred ON in dioxane/H₂O (4:1, 25 mL) at 100° C. under an inert atmosphere. The OL (water/EtOAc) was dried, concentrated, and purified by FC (CHCl₃/MTBE 1:0 to 0:1) to give Int. 1Z15 tert-butyl 4-(6-(dimethylcarbamoyl)pyridazin-3-yl)-3,6-dihydropyridine-1(2H)-carboxylate (1 g). Int. 1Z15 (1 g) was stirred 1 h in 1,1,1,3,3,3-hexafluoro-2-propanol/10M HCl (19:1, 10.5 mL). The mixture was diluted with MTBE to precipitate Int. 1Z16 N,N-dimethyl-6-(1,2,3,6-tetrahydro-pyridin-4-yl)pyridazine-3-carboxamide (0.8 g, HCl salt). Ints. 1J1/1Z16 (0.2 g/0.3 g) were stirred 12 h in ACN/Et₃N (48:1, 20.4 mL). The mixture was concentrated and HPLC-purified to give Int. 1Z14 6-(1-((4-bromo-1-methyl-1H-pyrrolo[2,3-b]pyridin-2-yl)methyl)-1,2,3,6-tetrahydropyridin-4-yl)-N,N-dimethylpyridazine-3-carboxamide (70 mg).

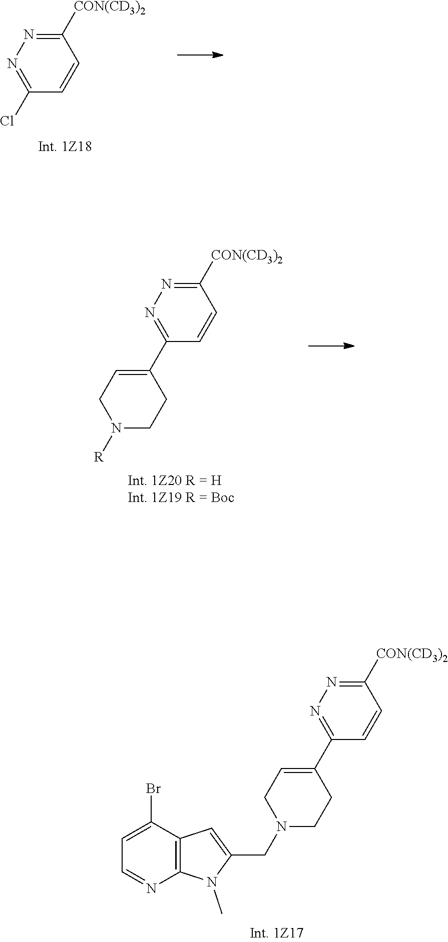

6-Chloropyridazine-3-carboxylic acid (1 g) was stirred in DCM/SOCl₂ (40:1, 20.5 mL) for 2 h at 40° C. and concentrated. Bis(methyl-d₃)amine hydrochloride (0.61 g) in DCM (20 mL) and Et₃N (2.6 mL) were added and the mixture was stirred ON. The mixture was concentrated and purified by FC (CHCl₃/ACN 1:0 to 1:1) to give Int. 1Z18 6-chloro-N,N-bis(methyl-d₃)pyridazine-3-carboxamide (1.2 g). Int. 1Z18 (1.2 g), tert-butyl 4-(4,4,5,5-tetramethyl-1,3,2-dioxaborolan-2-yl)-3,6-dihydropyridine-1(2H)-carboxylate (2.1 g), K₂CO₃ (3.5 g), and PdDPPFCl₂-DCM (0.26 g) were stirred ON in dioxane/H₂O (4:1, 25 mL) at 100° C. under an inert atmosphere. The OL (water/EtOAc) was dried, concentrated, and purified by FC (CHCl₃/MTBE 1:0 to 0:1) to give Int. 1Z19 tert-butyl 4-(6-(bis-(methyl-d₃)carbamoyl)-pyridazin-3-yl)-3,6-dihydropyridine-1(2H)-carboxylate (0.55 g). Int. 1Z19 (0.55 g) was stirred 1 h in 1,1,1,3,3,3-hexafluoro-2-propanol/10M HCl (26:1, 5.2 mL). The mixture was diluted with MTBE to precipitate Int. 1Z20 N,N-bis(methyl-d₃)-6-(1,2,3,6-tetrahydropyridin-4-yl)-pyridazine-3-carboxamide (0.2 g, HCl salt). Ints. 1J1/1Z20 (0.2 g/0.2 g) were stirred 12 h in ACN/Et₃N (48:1, 20.4 mL). The mixture was concentrated and HPLC-purified to give Int. 1Z17 6-(1-((4-bromo-1-methyl-1H-pyrrolo[2,3-b]-pyridin-2-yl)methyl)-1,2,3,6-tetrahydropyridin-4-yl)-N,N-bis(methyl-d₃)pyridazine-3-carboxamide (50 mg).

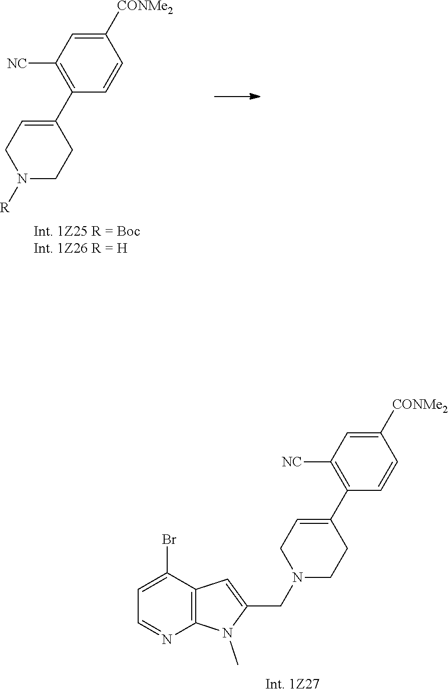

4-Chloro-3-cyanobenzoic acid (3.4 g) and di(1H-imidazol-1-yl)methanone (3.0 g) were refluxed 0.3 h in dioxane (50 mL). Dimethylamine hydrochloride (1.5 g) was added and stirring continued 10 h. The mixture was concentrated. The OL (aq. NaHSO₄/CHCl₃) was concentrated to give Int. 1Z24 4-chloro-3-cyano-N,N-dimethyl-benzamide (0.87 g). Int. 1Z24 (1 g), tert-butyl 4-(4,4,5,5-tetramethyl-1,3,2-dioxaborolan-2-yl)-3,6-dihydropyridine-1(2H)-carboxylate (1.6 g), K₂CO₃ (2.0 g), and PdDPPFCl₂-DCM (0.20 g) were stirred in dioxane/H₂O (4:1, 20 mL) ON at 100° C. under an inert atmosphere. The mixture was filtered, concentrated, and purified by FC (CHCl₃/ACN 1:0 to 4:1) to give Int. 1Z25 tert-butyl 4-(2-cyano-4-(dimethyl-carbamoyl)phenyl)-3,6-dihydropyridine-1(2H)-carboxylate (0.44 g). Int. 1Z25 (0.44 g) was stirred ON in 2.8M HCl in dioxane (10 mL). The mixture was concentrated to give Int. 1Z26 3-cyano-N,N-dimethyl-4-(1,2,3,6-tetrahydropyridin-4-yl)benzamide (0.3 g, HCl salt). Ints. 1J1/1Z26 (0.3 g/0.3 g) and K₂CO₃ (0.5 g) were stirred 11 h in DMF (10 mL). The mixture was diluted with water to precipitate Int. 1Z27 4-(1-((4-bromo-1-methyl-1H-pyrrolo[2,3-b]pyridin-2-yl)methyl)-1,2,3,6-tetrahydropyridin-4-yl)-3-cyano-N,N-dimethylbenzamide (0.12 g).

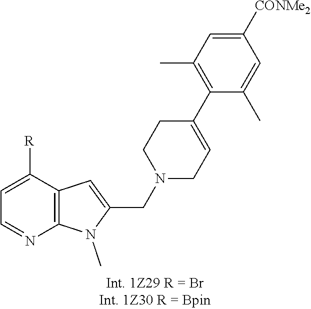

Ints. 1J1/RR44 (0.3 g/0.3 g) were stirred 12 h in ACN/Et₃N (29:2, 10.7 mL). The mixture was concentrated and HPLC-purified to give Int. 1Z29 4-(1-((4-bromo-1-methyl-1H-pyrrolo[2,3-b]pyridin-2-yl)methyl)-1,2,3,6-tetrahydropyridin-4-yl)-N,N,3,5-tetramethylbenzamide (0.3 g). Int. 1Z29 (0.3 g), bis(pinacolato)diboron (0.4 g), KOAc (0.5 g), and PdDPPFCl₂-DCM (50 mg) were stirred 12 h in dioxane (10 mL) at 80° C. under an inert atmosphere. The OL (water/EtOAc) was concentrated to give Int. 1Z30 N,N,3,5-tetramethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.3 g).

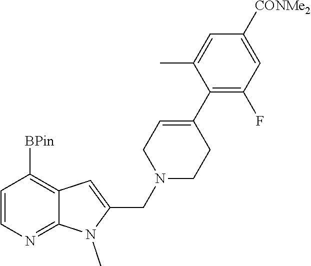

Int. 1Z50 (0.75 g), bis(pinacolato)diboron (0.78 g), PdDPPFCl₂-DCM (0.13 g), and KOAc (0.45 g) were stirred 1.3 h in dioxane (10 mL) at 110° C. under an inert atmosphere under MW conditions. The mixture was filtered through celite and concentrated to give Int. 1Z31 3-fluoro-N,N,5-trimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.85 g).

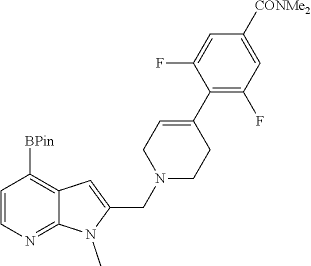

Int. 1Z65 (0.50 g), bis(pinacolato)diboron (0.52 g), PdDPPFCl₂-DCM (83 mg), and KOAc (0.30 g) were stirred 1 h in dioxane (10 mL) at 110° C. under an inert atmosphere under MW conditions. The mixture was filtered through celite, concentrated, and washed with pentane to give Int. 1Z33 3,5-difluoro-N,N-dimethyl-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.50 g).

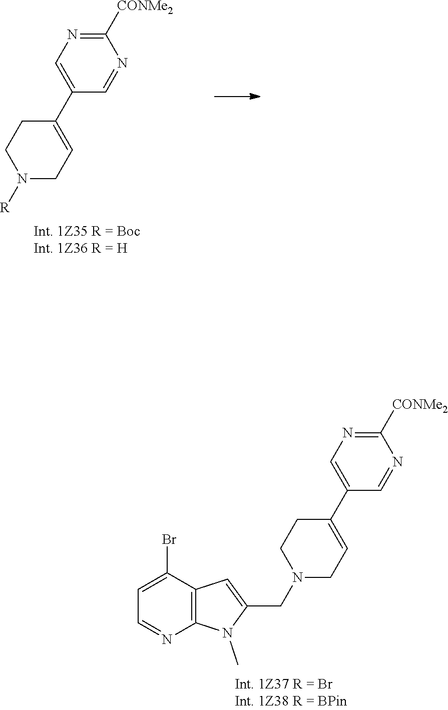

5-Bromo-N,N-dimethylpyrimidine-2-carboxamide (1.3 g), tert-butyl 4-(4,4,5,5-tetramethyl-1,3,2-dioxaborolan-2-yl)-3,6-dihydropyridine-1(2H)-carboxylate (1.9 g), K₂CO₃ (2.3 g), and PdDPPFCl₂-DCM (0.22 g) were stirred ON in dioxane/H₂O (4:1, 20 mL) at 100° C. under an inert atmosphere. The mixture was concentrated and purified by FC (MTBE/MeOH) to give Int. 1Z35 tert-butyl 4-(2-(dimethylcarbamoyl)pyrimidin-5-yl)-3,6-dihydropyridine-1(2H)-carboxylate (0.9 g). Int. 1Z35 (0.9 g) was stirred ON in 2.8M HCl in dioxane (10 mL). The mixture was concentrated to give Int. 1Z36 N,N-dimethyl-5-(1,2,3,6-tetrahydropyridin-4-yl)pyrimidine-2-carboxamide (0.65 g, HCl salt). Ints. 1J1/1Z36 (0.3 g/0.3 g) and K₂CO₃ (0.5 g) were stirred 11 h in DMF (10 mL). The mixture was diluted with water to precipitate Int. 1Z37 5-(1-((4-bromo-1-methyl-1H-pyrrolo[2,3-b]pyridin-2-yl)methyl)-1,2,3,6-tetrahydropyridin-4-yl)-N,N-dimethylpyrimidine-2-carboxamide (0.35 g). Int. 1Z37 (0.35 g), bis(pinacolato)diboron (0.70 g), PdDPPFCl₂-DCM (10 mg), and KOAc (0.40 g) were stirred 14 h in dioxane (10 mL) at 90° C. under an inert atmosphere. The OL (water/chloroform) was concentrated and HPLC-purified to give Int. 1Z38 N,N-dimethyl-5-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)pyrimidine-2-carboxamide (90 mg).

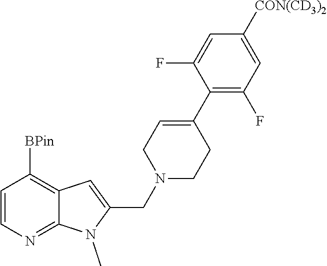

Int. 1Z65 (0.50 g), bis(pinacolato)diboron (0.52 g), PdDPPFCl₂-DCM (82 mg), and KOAc (0.30 g) were stirred 1 h in dioxane (6 mL) at 120° C. under MW conditions. The mixture was filtered through celite and concentrated to give Int. 1Z40 3,5-difluoro-N,N-bis(methyl-d₃)-4-(1-((1-methyl-4-(4,4,5,5-tetra-methyl-1,3,2-dioxaborolan-2-yl)-1H-pyrrolo[2,3-b]pyridin-2-yl)methyl)-1,2,3,6-tetrahydropyridin-4-yl)benzamide (0.60 g).

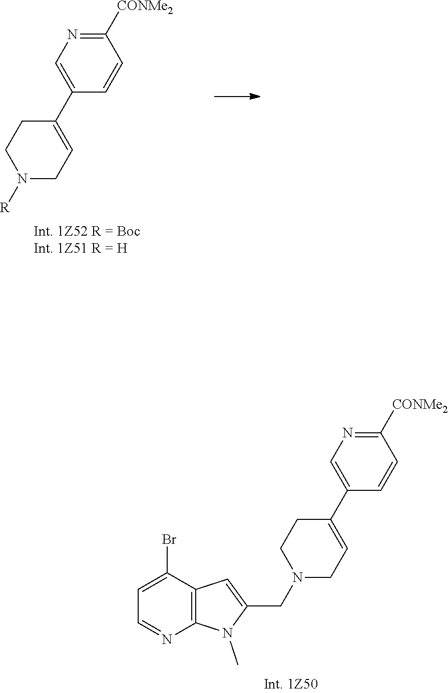

5-Bromo-N,N-dimethylpicolinamide (1.5 g), tert-butyl 4-(4,4,5,5-tetramethyl-1,3,2-dioxaborolan-2-yl)-3,6-dihydropyridine-1(2H)-carboxylate (2.2 g), K₂CO₃ (2.7 g), and PdDPPFCl₂-DCM (0.27 g) were stirred ON in dioxane/H₂O (4:1, 20 mL) ON 100° C. under an inert atmosphere. The mixture was concentrated and purified by FC (hexane/MTBE 1:0 to 0:1) to give Int. 1Z52 tert-butyl 6-(dimethyl-carbamoyl)-3′,6′-dihydro-[3,4′-bipyridine]-1′(2′H)-carboxylate (1 g). Int. 1Z51 (1 g) was stirred ON in 2.8M HCl in dioxane (10 mL). The mixture was concentrated to give Int. 1Z52 N,N-dimethyl-1′,2′,3′,6′-tetrahydro-[3,4′-bipyridine]-6-carboxamide (0.5 g, HCl salt). Ints. 1J1/1Z52 (0.6 g/0.5 g) and K₂CO₃ (0.9 g) were stirred 11 h in DMF (10 mL). The mixture was diluted with water to precipitate Int. 1Z50 1′-((4-bromo-1-methyl-1H-pyrrolo[2,3-b]pyridin-2-yl)methyl)-N,N-dimethyl-1′,2′,3′,6′-tetrahydro-[3,4′-bipyridine]-6-carboxamide (0.4 g).

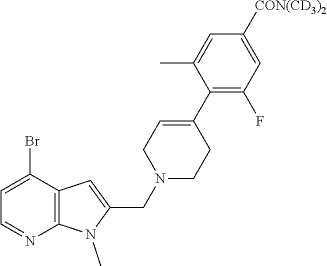

Int. 1AE9 (0.45 g), bis(pinacolato)diboron (0.35 g), PdDPPFCl₂-DCM (75 mg), and KOAc (0.27 g) were stirred 1 h in dioxane (5 mL) for 1 h at 110° C. under MW conditions. The mixture was filtered through celite and concentrated to give Int. 1Z53 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.40 g).

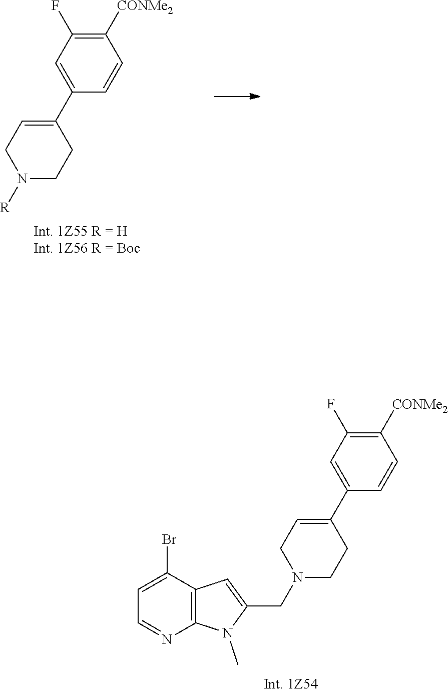

PdDPPFCl₂-DCM (0.25 g), 4-bromo-2-fluoro-N,N-dimethylbenzamide (1.5 g), tert-butyl 4-(4,4,5,5-tetramethyl-1,3,2-dioxaborolan-2-yl)-3,6-dihydropyridine-1(2H)-carboxylate (2.1 g), and K₂CO₃ (2.5 g) were stirred ON in dioxane/H₂O (4:1, 20 mL) at 100° C. under an inert atmosphere. The mixture was concentrated and purified by FC (hexane/MTBE 3:2 to 0:1) to give Int. 1Z55 tert-butyl 4-(4-(dimethyl-carbamoyl)-3-fluorophenyl)-3,6-dihydropyridine-1(2H)-carboxylate (1 g). Int. 1Z55 (1 g) was stirred ON in 2.8M HCl in dioxane (10 mL). The mixture was concentrated to give Int. 1Z56 2-fluoro-N,N-dimethyl-4-(1,2,3,6-tetrahydropyridin-4-yl)benzamide (0.5 g, HCl salt). Ints. 1J1/PP60 (0.5 g/0.5 g) and K₂CO₃ (0.8 g) were stirred 11 h in DMF (10 mL). The mixture was diluted with water to precipitate Int. 1Z54 4-(1-((4-bromo-1-methyl-1H-pyrrolo[2,3-b]pyridin-2-yl)methyl)-1,2,3,6-tetra-hydropyridin-4-yl)-2-fluoro-N,N-dimethylbenzamide (0.4 g).

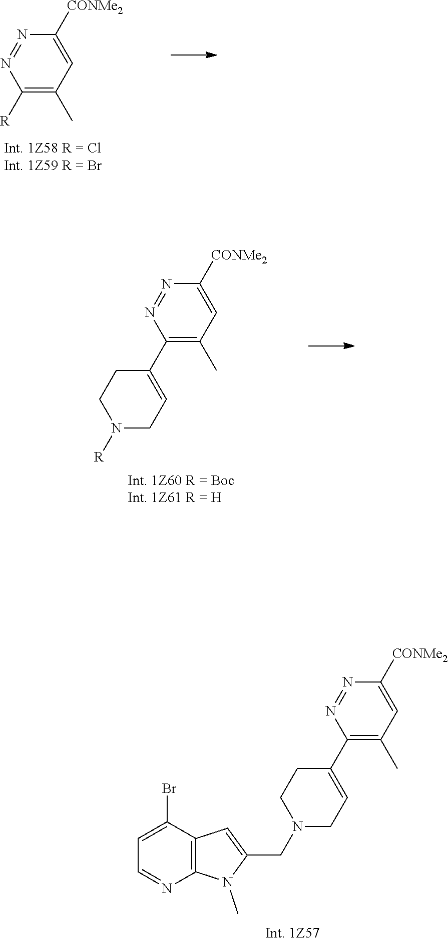

CDI (2.6 g) in THF (175 mL) was added dropwise to a mixture of 6-chloro-5-methylpyridazine-3-carboxylic acid (2 g) in THF (175 mL). The mixture was refluxed 3.5 h. Dimethylamine hydrochloride (2.8 g) and Et₃N (5.2 mL) were added and stirring continued 16 h at RT. The OL (brine/EtOAc) was concentrated to give Int. 1Z58 6-chloro-N,N,5-trimethylpyridazine-3-carboxamide (0.8 g). Int. 1Z58 was refluxed ON in bromotrimethylsilane (10 mL). The mixture was concentrated and purified by preparative TLC (hexane/EtOAc, 3:1) to give Int. 1Z59 6-bromo-N,N,5-trimethylpyridazine-3-carbox-amide (0.9 g). PdDPPFCl₂-DCM (0.3 g), Int. 1Z59 (0.9 g), tert-butyl 4-(4,4,5,5-tetramethyl-1,3,2-dioxa-borolan-2-yl)-3,6-dihydropyridine-1(2H)-carboxylate (1.0 g), and K₂CO₃ (2.0 g) were refluxed ON in dioxane/H₂O (15:4, 190 mL) under an inert atmosphere. The OL (aq. NaHCO₃/EtOAc) was dried, and concentrated to give Int. 1Z60 tert-butyl 4-(6-(dimethylcarbamoyl)-4-methylpyridazin-3-yl)-3,6-dihydropyridine-1(2H)-carboxylate (1.3 g). Int. 1Z60 (1.3 g) was stirred ON in 2.8M HCl in dioxane (10 mL). The residue after concentration was triturated in EtOAc to give Int. 1Z61 N,N,5-trimethyl-6-(1,2,3,6-tetrahydropyridin-4-yl)pyridazine-3-carboxamide (0.9, HCl salt). STAB (0.30 g) and Ints. 1AB8/1Z61 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z57 6-(1-((4-bromo-1-methyl-1H-pyrrolo[2,3-b]pyridin-2-yl)methyl)-1,2,3,6-tetrahydropyridin-4-yl)-N,N,5-trimethylpyridazine-3-carboxamide (0.19 g).

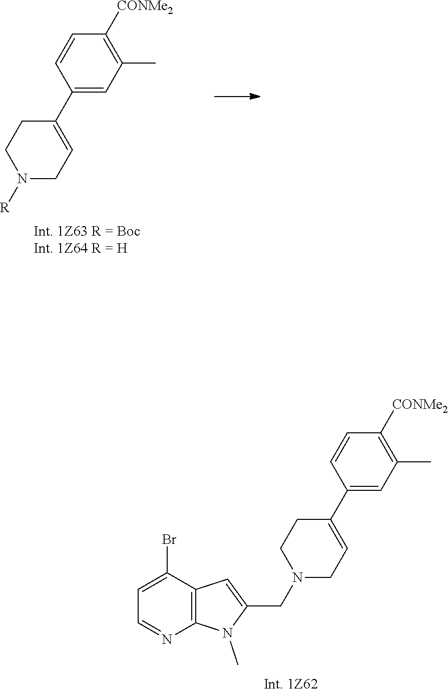

PdDPPFCl₂-DCM (1.1 g), 4-bromo-N,N,2-trimethylbenzamide (3.3 g), K₂CO₃ (5.7 g), and tert-butyl 4-(4,4,5,5-tetramethyl-1,3,2-dioxaborolan-2-yl)-3,6-dihydropyridine-1(2H)-carboxylate (5.9 g) were refluxed ON in dioxane/H₂O (15:4, 190 mL) under an inert atmosphere. The OL (aq. NaHCO₃/EtOAc) was dried and concentrated to give Int. 1Z63 tert-butyl 4-(4-(dimethylcarbamoyl)-3-methyl-phenyl)-3,6-dihydropyridine-1(2H)-carboxylate (4.1 g). Int. 1Z63 (4.1 g) was stirred ON in 2.8M HCl in dioxane (30 mL). The residue after concentration was triturated in EtOAc to give Int. 1Z64 N,N,2-trimethyl-4-(1,2,3,6-tetrahydropyridin-4-yl)benzamide (2.3 g, HCl salt). STAB (0.30 g) and Ints. 1AB8/1Z64 (0.1 g/0.1 g) were stirred ON in DCE/Et₃N (73:1, 10.1 mL) at 0° C. to RT. The residue after concentration was dissolved in THF, filtered through silica, and concentrated to give Int. 1Z62 4-(1-((4-bromo-1-methyl-1H-pyrrolo[2,3-b]pyridin-2-yl)methyl)-1,2,3,6-tetrahydropyridin-4-yl)-N,N,2-trimethylbenzamide (0.19 g).

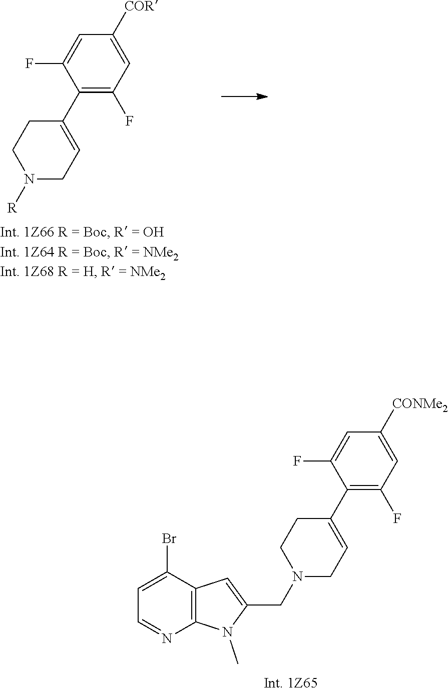

LiOH·H₂O (9.5 g) and Int. 3C18 (16 g) were stirred 16 h in THF/MeOH/H₂O (2:1:1, 300 mL) at 0° C. to RT. The residue after concentration was stirred in aq. citric acid to precipitate Int. 1Z66 4-(1-(tert-butoxy-carbonyl)-1,2,3,6-tetrahydropyridin-4-yl)-3,5-difluorobenzoic acid (10 g). Int. 1Z66 (5 g), HATU (8.4 g), and dimethylamine hydrochloride (1.8 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z67 tert-butyl 4-(4-(dimethylcarbamoyl)-2,6-difluorophenyl)-3,6-dihydropyridine-1(2H)-carboxylate (3.7 g). Int. 1Z67 (3.7 g) was stirred 16 h in 2M HCl in dioxane (60 mL) at 0° C. to RT. The mixture was concentrated to give Int. 1Z68 3,5-difluoro-N,N-dimethyl-4-(1,2,3,6-tetrahydropyridin-4-yl)benzamide (2.9 g, HCl salt). Ints. 1J1/1Z68 (0.35 g/0.49 g), KI (22 mg), and K₂CO₃ (0.56 g) were stirred 16 h in ACN (20 mL) at 0° C. to RT. The OL (water/EtOAc) was dried, concentrated, and purified by FC (PE/EtOAc 3:7) to give Int. 1Z65 4-(1-((4-bromo-1-methyl-1H-pyrrolo[2,3-b]pyridin-2-yl)methyl)-1,2,3,6-tetrahydropyridin-4-yl)-3,5-difluoro-N,N-dimethylbenzamide (0.35 g).

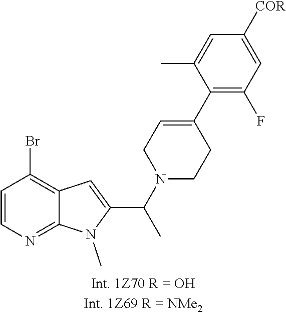

LiOH·H₂O (33 g) and Int. 1AF87 (76 g) were stirred 16 h in THE/MeOH/H2O (2:1:1, 1.5 L) at 0° C. to RT. ° C. to RT. The residue after concentration was stirred in aq. citric acid at 0° C. to precipitate Int. 1Z70 4-(1-(1-(4-bromo-1-methyl-1H-pyrrolo[2,3-b]pyridin-2-yl)ethyl)-1,2,3,6-tetrahydropyridin-4-yl)-3-fluoro-5-methylbenzoic acid (68 g). Int. 1Z70 (70 g), dimethylamine hydrochloride (36 g), and HATU (84 g) were stirred 12 h in DMF (300 mL) and DIPEA (180 mL) at 0° C. to RT. The mixture was diluted with water to precipitate a solid that was dried to give Int. 1Z69 4-(1-(1-(4-bromo-1-methyl-1H-pyrrolo-[2,3-b]pyridin-2-yl)ethyl)-1,2,3,6-tetrahydropyridin-4-yl)-3-fluoro-N,N,5-trimethylbenzamide (63 g).

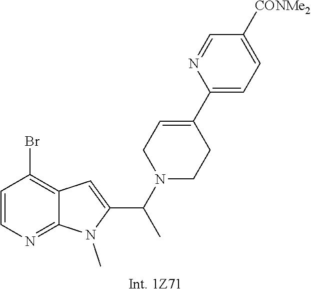

Ints. 1AF88/1S4 (1.5 g/1.5 g), KI (91 mg), and Cs₂CO₃ (8.9 g) were stirred 16 h in ACN (15 mL) at 0° C. to RT. The OL (water/EtOAc) was dried, concentrated, and purified by FC (DCM/MeOH 19:1) to give Int. 1Z71 1′-(1-(4-bromo-1-methyl-1H-pyrrolo[2,3-b]pyridin-2-yl)ethyl)-N,N-dimethyl-1′,2′,3′,6′-tetrahydro-[2,4′-bipyridine]-5-carboxamide (0.50 g).

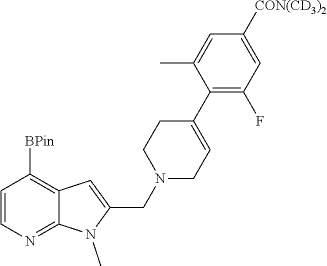

3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide Int. 1AE9 (0.45 g), bis(pinacolato)diboron (0.35 g), PdDPPFCl₂-DCM (75 mg), and KOAc (0.27 g) were stirred 1 h in dioxane (5 mL) at 110° C. under MW conditions. The mixture was filtered through celite and concentrated to give Int. 1Z73 3-fluoro-5-methyl-N,N-bis(methyl-d₃)-4-(1-((1-methyl-4-(4,4,5,5-tetramethyl-1,3,2-dioxaborolan-2-yl)-1H-pyrrolo[2,3-b]pyridin-2-yl)methyl)-1,2,3,6-tetrahydropyridin-4-yl)benzamide (0.40 g).

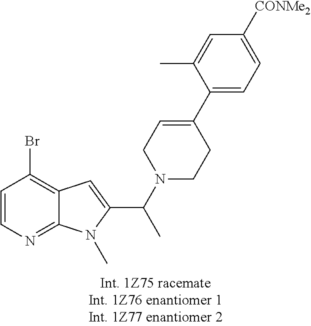

Int. 1AE38 (3.2 g) and Cs₂CO₃ (18 g) were stirred 0.3 h in ACN (20 mL). Int. 1AF88 (3 g) and KI (0.91 g) were added and stirring continued 12 h at 0° C. to RT. The OL (water/EtOAc) was dried, concentrated, and purified by FC (hexane/EtOAc 1:9) to give Int. 1Z75 4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide (0.90 g). Int. 1Z75 (0.90 g) was resolved by SFC on a SFC-150-022 instrument fitted with a Chiral ART Amylose-C NEO 250×30 mm 5 μm column operated at 30° C. and an eluent of 80% CO₂ and 20% MeOH at a (100 g/min) and a back pressure of 100 bar to give Int. 1Z76 (0.15 g, first peak) and Int. 1Z77 (0.15 g, second peak) (S)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide and (R)-4-(1-(1-(4-bromo-1-methyl-1H-pyrrolo[2,3-b]pyridin-2-yl)ethyl)-1,2,3,6-tetrahydropyridin-4-yl)-N,N,3-trimethylbenzamide. The absolute configurations of these compounds were not determined.

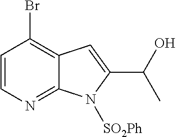

3M CH₃MgCl in THF (32 mL) was added slowly to a solution of Int. 1A6 (11.5 g) in THF (310 mL) at −78° C. The mixture was stirred 0.5 h at −78° C. to RT. The OL (aq. NH₄Cl/EtOAc) was dried, and concentrated to give Int. 1Z79 1-(4-bromo-1-(phenylsulfonyl)-1H-pyrrolo[2,3-b]pyridin-2-yl)ethan-1-ol (12 g).

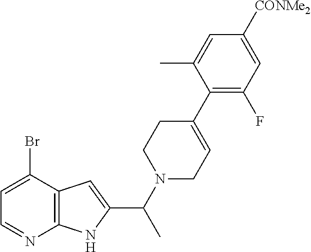

SOCl₂ (0.18 mL) was added to a solution of Int. 1Z79 (0.1 g) in toluene (3 mL). The mixture was stirred 0.5 h and concentrated. Int. 1AE12′ (90 mg), KI (20 mg), and Cs₂CO₃ (0.40 g) were stirred ON in a solution of the residue in ACN (5 mL). The mixture was HPLC-purified to give Int. 1Z80 4-(1-(1-(4-bromo-1H-pyrrolo[2,3-b]pyridin-2-yl)ethyl)-1,2,3,6-tetrahydropyridin-4-yl)-3-fluoro-N,N,5-trimethylbenzamide (13 mg).

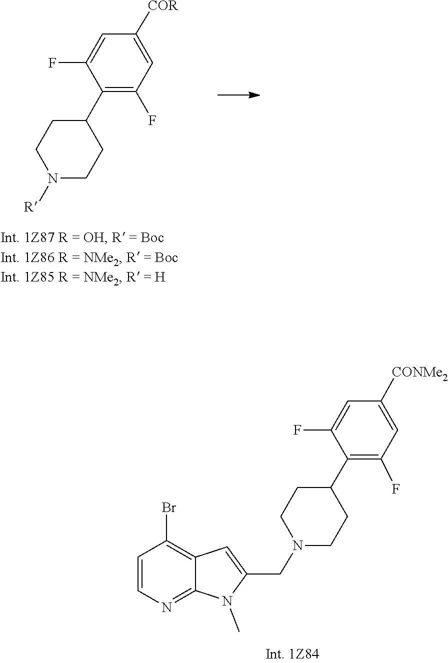

LiOH—H₂O (9.8 g) and Int. 3C₁₇ (16.5 g) were stirred 16 h in THF/MeOH/H₂O (2:1:1, 600 mL) at 0° C. to RT. The residue after concentration was stirred in aq. citric acid, filtered, and dried to give Int. 1Z87 4-(1-(tert-butoxycarbonyl)piperidin-4-yl)-3,5-difluorobenzoic acid (11.5 g). HATU (8.3 g), Int. 1Z87 (5 g), and dimethylamine hydrochloride (1.8 g) were stirred 16 h in DMF (30 mL) and DIPEA (13 mL) 0° C. to RT. The OL (water/EtOAc) was dried, concentrated, and purified by FC (PE/EtOAc 2:1) to give Int. 1Z86 tert-butyl 4-(4-(dimethylcarbamoyl)-2,6-difluorophenyl)piperidine-1-carboxylate (3.2 g). Int. 1Z86 (3.2 g) was stirred 16 h in 2M HCl in dioxane (60 mL) at 0° C. to RT. The mixture was concentrated to give Int. 1Z85 3,5-difluoro-N,N-dimethyl-4-(piperidin-4-yl)benzamide (2.3 g, HCl salt). Ints. 1J1/1Z85 (0.35 g/0.36 g) and K₂CO₃ (0.93 g) were stirred 16 h in ACN (10 mL) at 0° C. to RT. The mixture was filtered, concentrated, and purified by FC (PE/EtOAc 1:4) to give Int. 1Z84 4-(1-((4-bromo-1-methyl-1H-pyrrolo[2,3-b]pyridin-2-yl)methyl)piperidin-4-yl)-3,5-difluoro-N,N-dimethyl-benzamide (0.40 g).

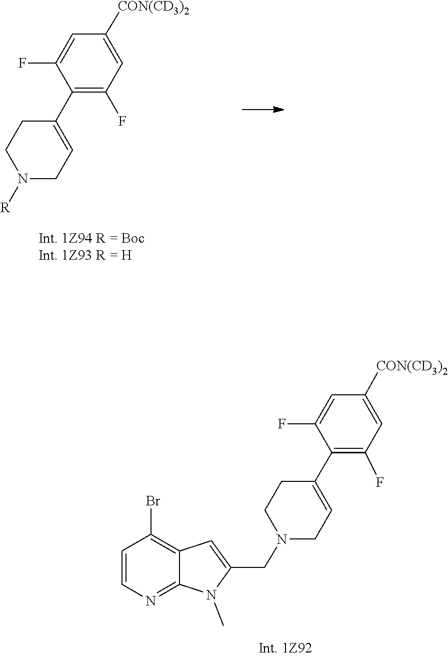

Int. 1Z66 (5 g), bis(methyl-d₃)amine hydrochloride (1.5 g), and HATU (8.4 g) were stirred 16 h in DMF (40 mL) and DIPEA (13 mL) at 0° C. to RT. The OL (water/EtOAc) was dried, concentrated, and purified by FC (PE/EtOAc 2:1) to give Int. 1Z94 tert-butyl 4-(4-(bis(methyl-d₃)carbamoyl)-2,6-difluoro-phenyl)-3,6-dihydropyridine-1(2H)-carboxylate (3.5 g). Int. 1Z94 (3.6 g) was stirred 16 h in 2M HCl in dioxane (60 mL) at 0° C. to RT. The mixture was concentrated to give Int. 1Z93 3,5-difluoro-N,N-bis-(methyl-d₃)-4-(1,2,3,6-tetrahydropyridin-4-yl)benzamide (2.7 g, HCl salt). Ints. 1J1/1Z93 (0.40 g/0.57 g), KI (77 mg), and K₂CO₃ (0.64 g) were stirred 16 h in ACN (20 mL) at 0° C. to RT. The OL (water/EtOAc) was dried, concentrated and, purified by FC (PE/EtOAc 3:7) to give Int. 1Z92 4-(1-((4-bromo-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide (0.41 g).

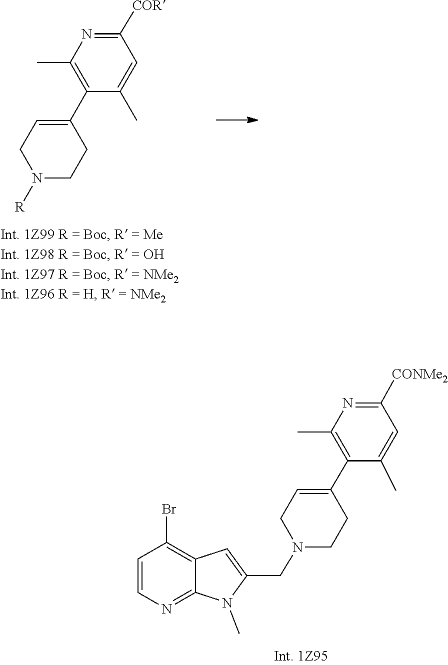

PdDPPFCl₂-DCM (0.2 g), methyl 5-bromo-4,6-dimethylpicolinate (1.1 g), K₂CO₃ (2 g), and tert-butyl 4-(4,4,5,5-tetramethyl-1,3,2-dioxaborolan-2-yl)-3,6-dihydropyridine-1(2H)-carboxylate (1.5 g) were stirred 12 h in dioxane (8:1, 45 mL) at 80° C. under an inert atmosphere. The mixture was filtered and concentrated to give Int. 1Z99 1′-(tert-butoxycarbonyl)-2,4-dimethyl-1′,2′,3′,6′-tetrahydro-[3,4′-bipyridine]-6-carboxylic acid (1.4 g). LiOH—H₂O (0.3 g) and Int. 1Z99 (1.4 g) were stirred 12 h in MeOH/H₂O (1:1, 60 mL). The mixture was concentrated to give Int. 1Z98 1′-(tert-butoxycarbonyl)-2,4-dimethyl-1′,2′,3′,6′-tetrahydro-[3,4′-bipyridine]-6-carboxylic acid (1.3 g, Li salt). Int. 1Z98 (1.4 g), dimethylamine hydrochloride (0.4 g) and HATU (1.7 g) were stirred 12 h in DMF (50 mL) and Et₃N (2.8 mL). The mixture was concentrated. The OL (water/EtOAc) was concentrated to give Int. 1Z97 tert-butyl 6-(dimethylcarbamoyl)-2,4-dimethyl-3′,6′-dihydro-[3,4′-bipyridine]-1′(2′H)-carboxylate (0.95 g). Int. 1Z97 (0.95 g) was stirred ON in MeOH/2.8M HCl in dioxane (1:1, 40 mL). The mixture was concentrated to give Int. 1Z96 N,N,2,4-tetramethyl-1′,2′,3′,6′-tetrahydro-[3,4′-bipyridine]-6-carboxamide (0.7 g, HCl salt). Int. 1AF89 (0.3 g) was stirred 0.5 h in toluene (20 mL) and SOCl₂ (0.61 mL) at 80° C. The mixture was concentrated. The residue, Int. 1Z96 (0.5 g), and Cs₂CO₃ (0.3 g) were stirred 12 h in DMF (10 mL). The OL (water/EtOAc) was concentrated and HPLC-purified to give Int. 1Z95 1′-(1-(4-bromo-1-methyl-1H-pyrrolo[2,3-b]pyridin-2-yl)ethyl)-N,N,2,4-tetramethyl-1′,2′,3′,6′-tetrahydro-[3,4′-bipyridine]-6-carboxamide (0.5 g).

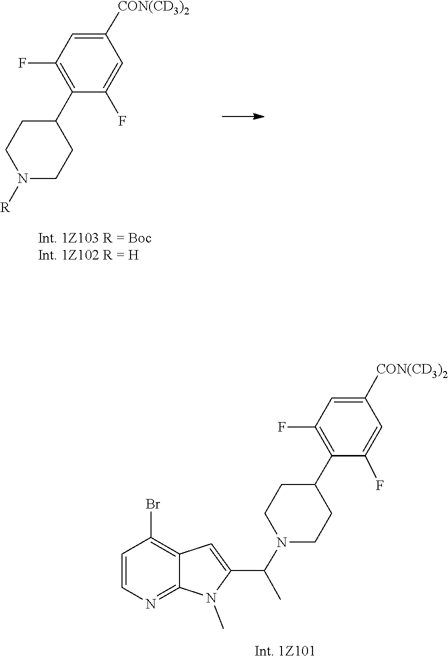

Int. 1Z87 (2 g), bis(methyl-d₃)amine hydrochloride (0.62 g), and HATU (3.3 g) were stirred 16 h in DMF (30 mL) and DIPEA (5.1 mL) at 0° C. to RT. The OL (water/EtOAc) was dried, concentrated, and purified by FC (PE/EtOAc 2:1) to give Int. 1Z103 tert-butyl 4-(4-(bis(methyl-d₃)carbamoyl)-2,6-difluorophenyl)-piperidine-1-carboxylate (1.4 g). Int. 1Z103 (1.4 g) was stirred 16 h in 2.2M HCl in dioxane (18 mL) at 0° C. to RT. The mixture was concentrated to give Int. 1Z102 3,5-difluoro-N,N-bis(methyl-d₃)-4-(piperidin-4-yl)benzamide (1.1 g, HCl salt). Ints. 1J1/1Z102 (0.50 g/0.60 g), KI (0.16 g), and K₂CO₃ (1.3 g) were stirred 16 h in ACN (20 mL) at 0° C. to RT. The OL (water/EtOAc) was concentrated and purified by FC (hexane/EtOAc 1:4) to give Int. 1Z101 4-(1-((4-bromo-1-methyl-1H-pyrrolo[2,3-b]pyridin-2-yl)methyl)piperidin-4-yl)-3,5-difluoro-N,N-bis(methyl-d₃)benzamide (0.45 g).

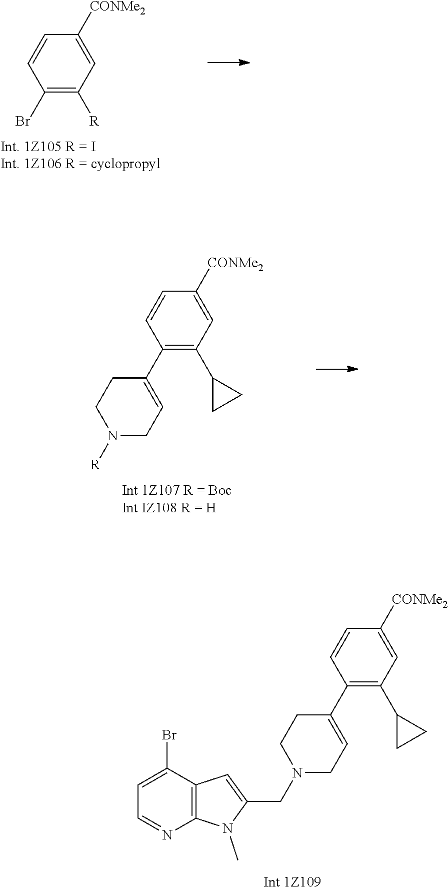

NaIO₄ (2.6 g) was added portion-wise to a mixture of I₂ (8.9 g) in sulfuric acid (100 mL). The mixture was stirred 0.5 h before 4-bromo-N,N-dimethylbenzamide (17 g) was added and stirring continued for 18 h. The mixture was diluted with water to precipitate a solid that was dried and crystallized from CCl₄ to give Int. 1Z105 4-bromo-3-iodo-N,N-dimethylbenzamide (17 g). PdDPPFCl₂-DCM (2.0 g), Int. 1Z105 (17 g), cyclopropylboronic acid (5.0 g), and K₂CO₃ (20 g) were refluxed ON in dioxane/H₂O (3:1, 200 mL) under an inert atmosphere. The mixture was concentrated and purified by FC (CHCl₃ to CHCl₃/ACN 1:1) to give Int. 1Z106 4-bromo-3-cyclopropyl-N,N-dimethylbenzamide (12 g). PdDPPFCl₂-DCM (1.9 g), Int. 1Z106 (12 g), tert-butyl 4-(4,4,5,5-tetramethyl-1,3,2-dioxaborolan-2-yl)-3,6-dihydropyridine-1(2H)-carboxylate (21 g), and K₂CO₃ (19 g) were refluxed ON in dioxane/H₂O (6:1, 140 mL) under an inert atmosphere. The mixture was concentrated and purified by FC (CHCl₃ to CHCl₃/ACN 1:1) to give Int. 1Z107 tert-butyl 4-(2-cyclopropyl-4-(dimethylcarbamoyl)phenyl)-3,6-dihydropyridine-1(2H)-carboxylate (4.1 g). Int. 1Z107 (4.1 g) was stirred in ON 4M HCl in dioxane (50 mL). The mixture was concentrated and triturated in EtOAc to give Int. 1Z108 3-cyclopropyl-N,N-dimethyl-4-(1,2,3,6-tetrahydropyridin-4-yl)benzamide (0.10 g, HCl salt). STAB (0.30 g) and Ints. 1AB8/1Z108 (0.1 g/0.1 g) were stirred ON in DCE/Et₃N (73:1, 10.1 mL) at 0° C. to RT. The mixture was concentrated, dissolved in THF, filtered through silica, and concentrated to give Int. 1Z109 4-(1-((4-bromo-1-methyl-1H-pyrrolo[2,3-b]pyridin-2-yl)methyl)-1,2,3,6-tetrahydropyridin-4-yl)-3-cyclopropyl-N,N-dimethylbenzamide (0.19 g).

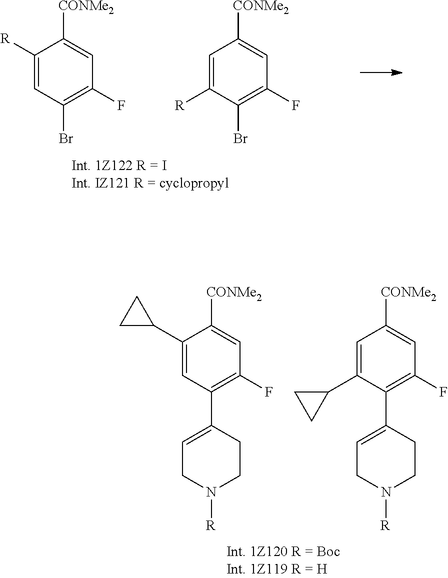

NaIO₄ (2.3 g) was added to a mixture of I₂ (8.0 g) in sulfuric acid (100 mL) portion-wise and stirred 0.5 h. 4-Bromo-3-fluoro-N,N-dimethylbenzamide (17 g) was added and stirring continued 18 h. The reaction mixture was diluted with water to precipitate a solid that was dried and precipitated from CCl₄ to give Int. 1Z122, a mixture of 4-bromo-5-fluoro-2-iodo-N,N-dimethylbenzamide and 4-bromo-3-fluoro-5-iodo-N,N-dimethylbenzamide mixture (13 g). PdDPPFCl₂-DCM (1.4 g), Int. 1Z122 (13 g), cyclopropylboronic acid (3.6 g), and K₂CO₃ (14 g) were refluxed ON in dioxane/H₂O (3:1, 200 mL) under an inert atmosphere. The mixture was concentrated and purified by FC (CHCl₃ to CHCl₃/ACN 1:1) to give Int. 1Z121, a mixture of 4-bromo-2-cyclopropyl-5-fluoro-N,N-dimethylbenzamide and 4-bromo-3-cyclopropyl-5-fluoro-N,N-dimethylbenzamide mixture (7 g). PdDPPFCl₂-DCM (1.0 g), Int. 1Z121 (7 g), tert-butyl 4-(4,4,5,5-tetramethyl-1,3,2-dioxaborolan-2-yl)-3,6-dihydropyridine-1(2H)-carboxylate (11 g), and K₂CO₃ (10 g) were refluxed ON in dioxane/H₂O (6:1, 140 mL) under an inert atmosphere. The mixture was concentrated and purified by FC (CHCl₃ to CHCl₃/ACN 1:1) to give Int. 1Z120, a mixture of tert-butyl 4-(5-cyclopropyl-4-(dimethylcarbamoyl)-2-fluorophenyl)-3,6-dihydropyridine-1(2H)-carboxylate and tert-butyl 4-(2-cyclopropyl-4-(dimethylcarbamoyl)-6-fluoro-phenyl)-3,6-dihydropyridine-1(2H)-carboxylate mixture (2.6 g). Int. 1Z120 (2.6 g) was stirred in ON 4M HCl in dioxane (50 mL). The mixture was concentrated and triturated in EtOAc to give Int. 1Z119 2-cyclopropyl-5-fluoro-N,N-dimethyl-4-(1,2,3,6-tetrahydropyridin-4-yl)benzamide and 3-cyclopropyl-5-fluoro-N,N-dimethyl-4-(1,2,3,6-tetrahydropyridin-4-yl)benzamide mixture (2.0 g, HCl salt).

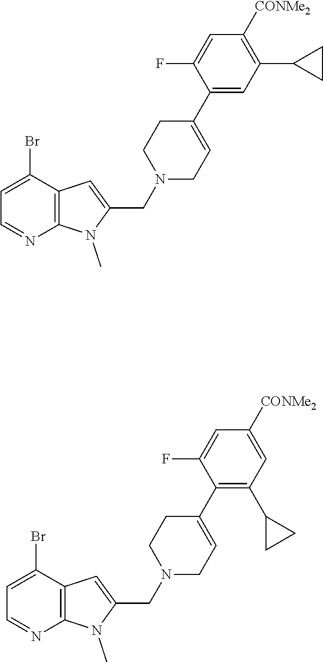

STAB (0.30 g) and Ints. 1AB8/1Z119 (0.1 g/0.1 g) were stirred ON in DCE/Et₃N (73:1, 10.1 mL) at 0° C. to RT. The residue after concentration dissolved in THF, filtered through silica, and concentrated to give a mixture of Ints. 1Z123/1Z124 4-(1-((4-bromo-1-methyl-1H-pyrrolo[2,3-b]pyridin-2-yl)methyl)-1,2,3,6-tetrahydropyridin-4-yl)-2-cyclopropyl-5-fluoro-N,N-dimethylbenzamide and 4-(1-((4-bromo-1-methyl-1H-pyrrolo[2,3-b]pyridin-2-yl)methyl)-1,2,3,6-tetrahydropyridin-4-yl)-3-cyclopropyl-5-fluoro-N,N-dimethylbenzamide (0.17 g).

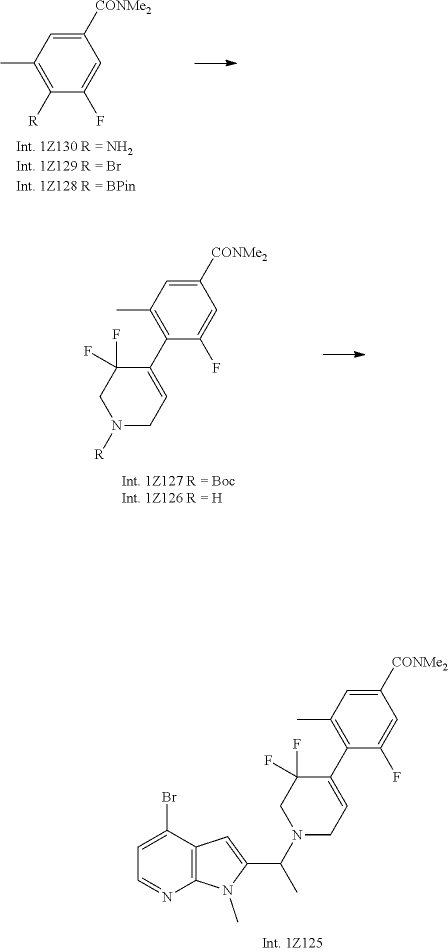

N-Iodosuccinimide (196 g) and 2-fluoro-6-methylaniline (100 g) were stirred 2 h in ACN (2.0 L) at 0° C. to RT. The OL (aq. Na₂S₂O₃/EtOAc) was dried, and concentrated to give Int. 1Z131 2-fluoro-4-iodo-6-methylaniline (180 g). Int. 1Z131 (30 g), dimethylamine hydrochloride (24 g), and PdDPPFCl₂-DCM (4.9 g) were stirred 16 h in dioxane/Et₃N (14:3, 364 mL) at 85° C. under 200 psi of CO. The mixture was filtered and concentrated. The OL (1M HCl/EtOAc) was neutralized to pH ˜8 with Na₂CO₃. The OL (water/EtOAc) was dried, and concentrated to give Int. 1Z130 4-amino-3-fluoro-N,N,5-trimethylbenzamide (17 g). CuBr₂ (85 g) and 1Z130 (15 g) were stirred 16 h in ACN (300 mL) and ^{t}BuONO (14 mL) at 0-85° C. The OL (aq. NH₃/PE) was dried, filtered and concentrated to give Int. 1Z129 4-bromo-3-fluoro-N,N,5-trimethylbenzamide (14 g). Int. 1Z129 (1 g), PdDPPFCl₂-DCM (0.31 g), bis(pinacolato)diboron (2.0 g), and KOAc (1.1 g) were stirred 1 h in dioxane (10 mL) at 110° C. under MW conditions. The mixture was filtered through celite, concentrated, and washed with pentane to give Int. 1Z128 3-fluoro-N,N,5-trimethyl-4-(4,4,5,5-tetramethyl-1,3,2-dioxaborolan-2-yl)benzamide (0.80 g). PdDPPFCl₂-DCM (0.11 g), Ints. 3E284/1Z128 (0.50 g/0.77 g), and Na₂CO₃ (0.43 g) were stirred 1.5 h in dioxane/H₂O (4:1, 10 mL) at 110° C. under MW conditions. The mixture was filtered. The OL (water/EtOAc) was concentrated and purified by FC (hexane/EtOAc 3:7) to give Int. 1Z127 tert-butyl 4-(4-(dimethylcarbamoyl)-2-fluoro-6-methylphenyl)-3,3-difluoro-3,6-dihydro-pyridine-1(2H)-carboxylate (65 mg). Int. 1Z127 (0.50 g) was stirred 16 h in DCM/4M HCl in dioxane (2:1, 6 mL) at 0° C. to RT. The mixture was concentrated to give Int. 1Z126 4-(3,3-difluoro-1,2,3,6-tetrahydro-pyridin-4-yl)-3-fluoro-N,N,5-trimethylbenzamide (0.40 g, HCl salt). Ints. 1AF88/1Z126 (0.40 g/0.59 g), KI (0.17 g), and Cs₂CO₃ (3.3 g) were stirred 12 h in ACN (10 mL) at 0° C. to RT. The mixture was filtered, concentrated, and purified by FC (hexane/EtOAc 4:1) to give Int. 1Z125 4-(1-(1-(4-bromo-1-methyl-1H-pyrrolo[2,3-b]-pyridin-2-yl)ethyl)-3,3-difluoro-1,2,3,6-tetrahydropyridin-4-yl)-3-fluoro-N,N,5-trimethylbenzamide (0.13 g).

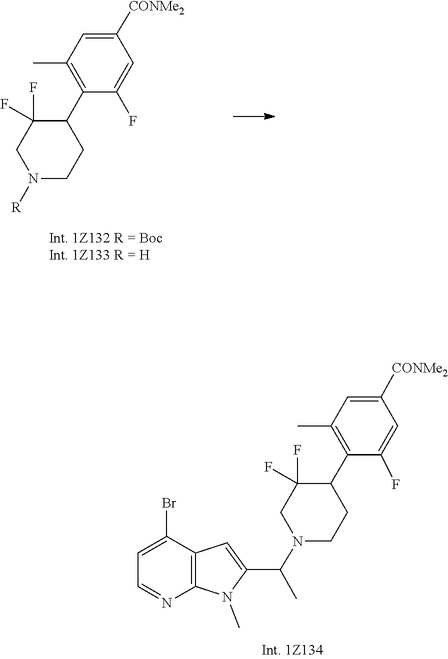

Int. 1Z127 (0.40 g) and PtO₂ (0.80 g) were hydrogenated 75 h at 70 psi in THF/EtOH/AcOH (8:8:1, 17 mL). The mixture was filtered and concentrated to give Int. 1Z132 tert-butyl 4-(4-(dimethyl-carbamoyl)-2-fluoro-6-methylphenyl)-3,3-difluoropiperidine-1-carboxylate (0.40 g). Int. 1Z133 4-(3,3-difluoropiperidin-4-yl)-3-fluoro-N,N,5-trimethylbenzamide (0.20 g, HCl salt) was prepared similarly to Int. 1Z126 from Int. 1Z132 (0.25 g). Ints. 1AF88/1Z133 (0.15 g/0.18 g), KI (64 mg), and Cs₂CO₃ (0.12 g) were stirred 16 h in ACN (10 mL) at 0° C. to RT. The OL (water/EtOAc) was dried, concentrated, and purified by FC (PE/EtOAc 1:1 to 1:2) to give Int. 1Z134 4-(1-(1-(4-bromo-1-methyl-1H-pyrrolo[2,3-b]pyridin-2-yl)ethyl)-3,3-difluoropiperidin-4-yl)-3-fluoro-N,N,5-trimethyl-benzamide (0.13 g).

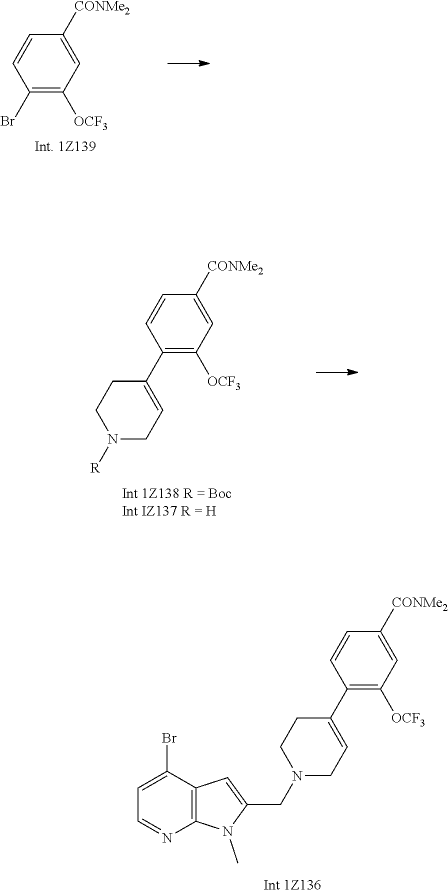

Di(1H-imidazol-1-yl)methanone (1.0 g) and 4-bromo-3-(trifluoromethoxy)benzoic acid (1.8 g) were refluxed 0.3 h in dioxane (50 mL). Dimethylamine hydrochloride (0.51 g) and Et₃N (5.2 mL) was added and stirring continued 10 h. The mixture was concentrated. The OL (aq. NaHSO₄/CHCl₃) was concentrated to give Int. 1Z139 4-bromo-N,N-dimethyl-3-(trifluoro(oxo)-λ6-methyl)benzamide (1.5 g). Int. 1Z139 (1.5 g), tert-butyl 4-(4,4,5,5-tetramethyl-1,3,2-dioxaborolan-2-yl)-3,6-dihydropyridine-1(2H)-carboxylate (1.6 g), K₂CO₃ (2.0 g), and PdDPPFCl₂-DCM (0.20 g) were stirred ON in dioxane/H₂O (4:1, 20 mL) at 100° C. under an inert atmosphere. The mixture was concentrated and purified by FC to give Int. 1Z138 tert-butyl 4-(4-(dimethylcarbamoyl)-2-(trifluoromethoxy)phenyl)-3,6-dihydro-pyridine-1(2H)-carboxylate (0.78 g). Int. 1Z138 (0.78 g) was stirred ON in 2.8M HCl in dioxane (10 mL). The mixture was concentrated to give Int. 1Z137 N,N-dimethyl-4-(1,2,3,6-tetrahydropyridin-4-yl)-3-(trifluoromethoxy)benzamide (0.43 g, HCl salt). Ints. 1J1/1Z137 (0.36 g/0.43 g) and K₂CO₃ (0.57 g) were stirred 11 h in DMF (10 mL). The mixture was diluted with water to precipitate Int. 1Z136 4-(1-((4-bromo-1-methyl-1H-pyrrolo[2,3-b]pyridin-2-yl)methyl)-1,2,3,6-tetrahydropyridin-4-yl)-N,N-dimethyl-3-(trifluoromethoxy)benzamide (0.14 g).

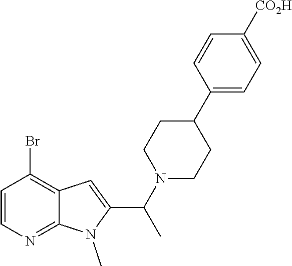 [SMILES: CC(c1cc2c(Br)ccnc2n1C)N1CCC(c2ccc(C(=O)O)cc2)CC1]

Int. C3. 4-(1-(1-(4-bromo-1-methyl-1H-pyrrolo[2,3-b]pyridin-2-yl)ethyl)piperidin-4-yl)benzoic acid (0.11 g) was prepared similarly to Int. 2C9 from Int. 3E37 (0.12 g).

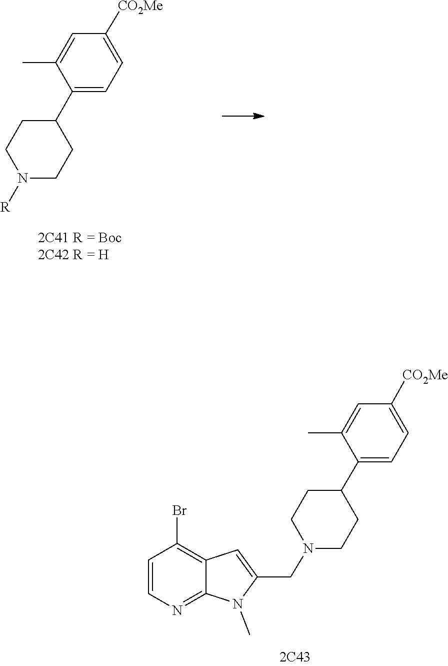

Methyl 4-bromo-3-methylbenzoate (2.5 g), tert-butyl 4-(4,4,5,5-tetramethyl-1,3,2-dioxaborolan-2-yl)-3,6-dihydropyridine-1(2H)-carboxylate (4.0 g), NaHCO₃ (2.8 g), and PdDPPFCl₂-DCM (0.45 g) were degassed in dioxane/water (10:1, 30 mL) and stirred ON at 100° C. The mixture was diluted with EtOAc (100 mL), filtered, concentrated, and purified by FC (hexane/EtOAc 9:1 to 85:15) to give Int. 2C41 tert-butyl 4-(4-methoxy-carbonyl-2-methyl-phenyl)-3,6-dihydro-2H-pyridine-1-carboxylate (3.2 g). Int. 2C41 (0.25 g) was stirred ON in DCM/4M HCl in dioxane (4:1, 10 mL) and concentrated. The residue was triturated in pentane to give Int. 2C42 methyl 3-methyl-4-(1,2,3,6-tetra-hydropyridin-4-yl)benzoate (0.19 g, HCl salt). Ints. 1AB8/2C42 (1.2 g/1.2 g, HCl salt) were stirred 4 h in DCE/DIPEA (2:2:1, 7.3 mL) at 0° C. to RT. STAB (1.9 g) was added and stirring continued ON. The OL (EtOAc/water) was dried, concentrated, and purified by FC (hexane/EtOAc 7:3) to give Int. 2C43 methyl 4-(1-((4-bromo-1-methyl-1H-pyrrolo[2,3-b]pyridin-2-yl)methyl)-1,2,3,6-tetrahydropyridin-4-yl)-3-methyl-benzoate (1.0 g).

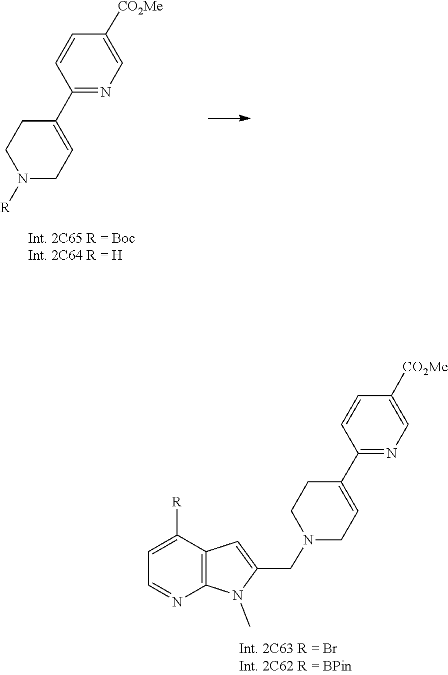

Methyl 6-bromonicotinate (5.0 g), tert-butyl 4-(4,4,5,5-tetramethyl-1,3,2-dioxaborolan-2-yl)-3,6-dihydropyridine-1(2H)-carboxylate (7.8 g), K₂CO₃ (3.0 g) and PdDPPFCl₂-DCM (1.9 g) were stirred in dioxane/H₂O (4:1, 100 mL) ON at 100° C. under an inert atmosphere. The OL (water/EtOAc) was dried, concentrated and purified by FC (hexane/MTBE 1:0 to 0:1) to give Int. 2C65 1′-(tert-butyl) 5-methyl 3′,6′-dihydro-[2,4′-bipyridine]-1′,5(2′H)-dicarboxylate (5.9 g). Int. 2C₆₅ (2.0 g) was stirred 16 h in 2M HCl in dioxane (40 mL), concentrated, and washed with Et₂O to give Int. 2C64 methyl 1′,2′,3′,6′-tetrahydro-[2,4′-bipyridine]-5-carboxylate (1.8 g, HCl salt). Ints. 1AB8/2C64 (616 mg/750 mg) and 4A MS were stirred ON in DCE/Et₃N (9:1, 11.1 mL). (AcO)₃BHNa (1.6 g) was added and stirring continued for 3 h before it was concentrated and HPLC-purified to give Int. 2C63 methyl 1′-((4-bromo-1-methyl-1H-pyrrolo[2,3-b]pyridin-2-yl)methyl)-1′,2′,3′,6′-tetrahydro-[2,4′-bipyridine]-5-carboxylate (0.57 g). Int. 2C63 (0.29 g), bis(pinacolato)diboron (0.18 g), KOAc (0.19 g), and PdDPPFCl₂-DCM (53 mg) were stirred ON in dioxane (10 mL) at 80° C. under an inert atmosphere. The residue after concentration was dissolved in THF, filtered through silica, and concentrated to give Int. 2C62 methyl 1′-((1-methyl-4-(4,4,5,5-tetramethyl-1,3,2-dioxaborolan-2-yl)-1H-pyrrolo[2,3-b]pyridin-2-yl)-methyl)-1′,2′,3′,6′-tetrahydro-[2,4′-bipyridine]-5-carboxylate (0.30 g).

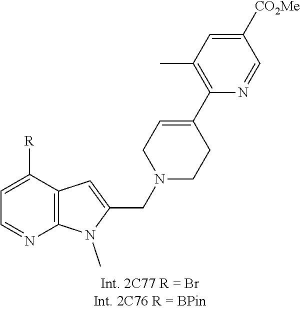

Ints. 1AB8/2C78 (0.50 g/1.0 g) and 4A MS were stirred ON in DCE/Et₃N (15:1, 15.9 mL). (AcO)₃BHNa (1.0 g) was added and stirring continued 3 h. The mixture was concentrated and HPLC-purified to give Int. 2C77 methyl 1′-((4-bromo-1-methyl-1H-pyrrolo[2,3-b]pyridin-2-yl)methyl)-3-methyl-1′,2′,3′,6′-tetrahydro-[2,4′-bipyridine]-5-carboxylate (0.61 g). Int. 2C77 (0.30 g), bis(pinacolato)diboron (0.30 mg), KOAc (0.30 mg) and PdDPPFCl₂-DCM (30 mg) were stirred ON in dioxane (3 mL) at 80° C. under an inert atmosphere. The residue after concentration was dissolved in THF, filtered through silica and concentrated to give Int. 2C76 methyl 3-methyl-1′-((1-methyl-4-(4,4,5,5-tetramethyl-1,3,2-dioxaborolan-2-yl)-1H-pyrrolo[2,3-b]pyridin-2-yl)methyl)-1′,2′,3′,6′-tetrahydro-[2,4′-bipyridine]-5-carboxylate (0.30 g).

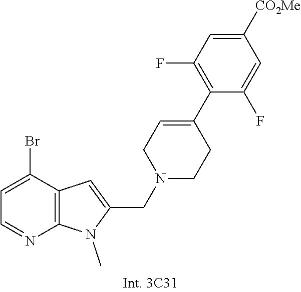 [SMILES: COC(=O)c1cc(F)c(C2=CCN(Cc3cc4c(Br)ccnc4n3C)CC2)c(F)c1]

Ints. 1AB8/3C25 (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, filtered, concentrated, and purified by FC (PE/EtOAc 3:2) to give Int. 3C31 methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

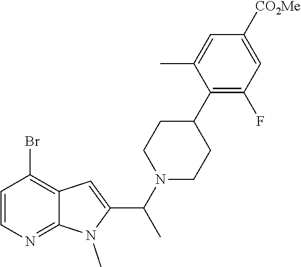 [SMILES: COC(=O)c1cc(C)c(C2CCN(C(C)c3cc4c(Br)ccnc4n3C)CC2)c(F)c1]

Ints. 1AF88/2G97 (2.0 g/2.3 g), KI (0.85 g) and Cs₂CO₃ (9.5 g) were mixed in ACN (20 mL) at 0° C. and stirred for 16 h at RT filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl)piperidin-4-yl)-3-fluoro-5-methylbenzoate (2.0 g).

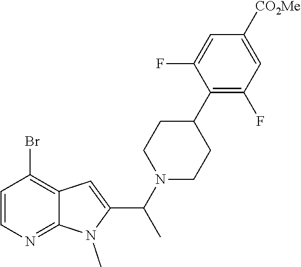 [SMILES: COC(=O)c1cc(F)c(C2CCN(C(C)c3cc4c(Br)ccnc4n3C)CC2)c(F)c1]

Int. 1AF89 (1.5 g) was stirred 0.3 h in toluene/SOCl₂ (7:3, 14.3 mL) at 90° C. The residue after concentration, Cs₂CO₃ (7.7 g), KI (0.49 g), and Int. 3C16 (2.1 g) were stirred 16 h in ACN (10 mL) at 0° C. to RT under an inert atmosphere. The reaction mixture was concentrated and triturated in water to precipitate a solid that was washed with water, dried, and purified by FC (PE/EtOAc 9:1 to 17:3) to give Int. 2G96 methyl 4-(1-(1-(4-bromo-1-methyl-1H-pyrrolo[2,3-b]pyridin-2-yl)ethyl)piperidin-4-yl)-3,5-difluorobenzoate (1.5 g).

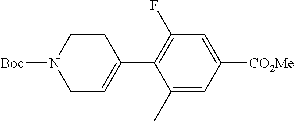 [SMILES: COC(=O)c1cc(C)c(C2=CCN(C(=O)OC(C)(C)C)CC2)c(F)c1]

Int. 1AF90 (15 g), Boc₂O (17 g) and DMAP (0.64 g) were mixed in DCM/Et₃N (4:1, 187 mL) at 0° C. and stirred 16 h and filtered. The OL (water/DCM) was dried, concentrated, and purified by FC (hexane/EtOAc 2:1) to give Int. 2G105 tert-butyl 4-(2-fluoro-4-(methoxycarbonyl)-6-methylphenyl)-3,6-dihydropyridine-1(2H)-carboxylate (14 g).

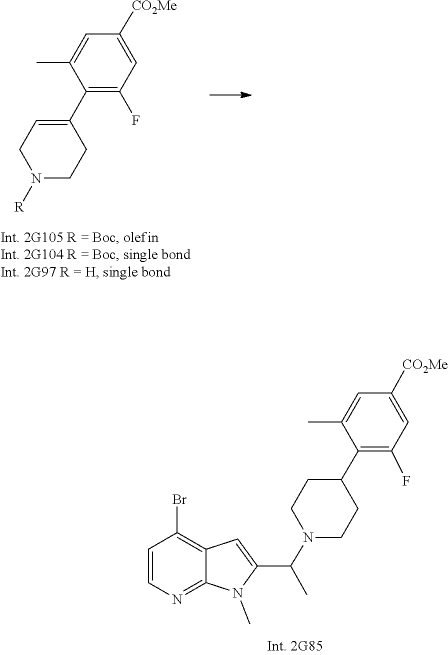

Int. 2G105 (14 g) and Pd/C (7 g) was hydrogenated 16 h in MeOH (140 mL), filtered, concentrated to give Int. 2G104 tert-butyl 4-(2-fluoro-4-(methoxycarbonyl)-6-methylphenyl)piperidine-1-carboxylate (13.2 g). Int. 2G104 (13.2 g) was stirred 16 h in DCM/4M HCl in dioxane (2:5, 105 mL) at 0° C. to RT. The mixture was concentrated and washed with pentane to give Int. 2G97 methyl 3-fluoro-5-methyl-4-(piperidin-4-yl)benzoate (9.0 g, HCl salt). Ints. 1AF88/2G97 (2.0 g/2.3 g), KI (0.85 g), and Cs₂CO₃ (9.5 g) were stirred 16 h in ACN (20 mL) at 0° C. to RT. The mixture was filtered and concentrated. The residue was purified by FC (hexane/EtOAc 4:1) to give Int. 2G85 methyl 4-(1-(1-(4-bromo-1-methyl-1H-pyrrolo[2,3-b]pyridin-2-yl)ethyl)piperidin-4-yl)-3-fluoro-5-methylbenzoate (2.0 g).

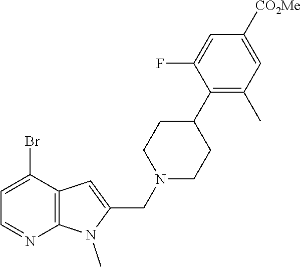 [SMILES: COC(=O)c1cc(C)c(C2CCN(Cc3cc4c(Br)ccnc4n3C)CC2)c(F)c1]

DIPEA (15 mL) was added to a solution of Ints. 1AB8/2G97 (8.0 g/12.5 g) in DCE/DMSO (5:1, 600 mL) at 0° C. and stirred for 16 h at RT. STAB (28 g) was added and stirring continued for 16 h. The OL (water/DCM/MeOH) was dried, filtered, concentrated and purified by FC (hexane/MeOH 4:1 to 2:1) to give Int. 2G115 methyl 4-(1-((4-bromo-1-methyl-1H-pyrrolo[2,3-b]pyridin-2-yl)methyl)piperidin-4-yl)-3-fluoro-5-methylbenzoate (8.0 g).

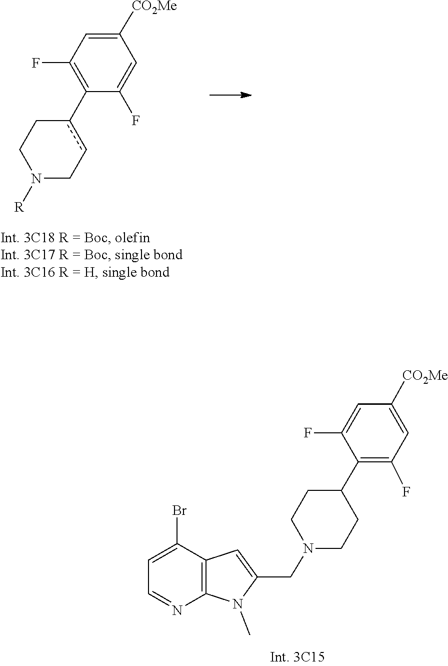

Methyl 4-bromo-3,5-difluorobenzoate (100 g), tert-butyl 4-(4,4,5,5-tetramethyl-1,3,2-dioxaborolan-2-yl)-3,6-dihydropyridine-1(2H)-carboxylate (160 g), PdDPPFCl₂-DCM (16 g), and NaHCO₃ (100 g) were stirred 16 h in dioxane/H₂O (10:3, 1.3 L) at 100° C. under an inert atmosphere. The mixture was filtered. The OL (brine/EtOAc) was dried, concentrated, and purified by FC (PE/EtOAc 1:0 to 9:1) to give Int. 3C18 tert-butyl 4-(2,6-difluoro-4-(methoxycarbonyl)phenyl)-3,6-dihydropyridine-1(2H)-carboxylate (45 g). Int. 3C18 (100 g) and 5% wet Pd/C (20 g) were stirred 16 h in MeOH (0.7 L) under an atmosphere of hydrogen. The mixture was filtered and concentrated to give Int. 3C17 tert-butyl 4-(2,6-difluoro-4-(methoxycarbonyl)phenyl)piperidine-1-carboxylate (90 g). Int. 3C17 (20 g) was stirred 16H in DCM/4M HCl in dioxane (2:1, 600 mL) at 0° C. to RT. The mixture was concentrated and washed with Et₂O to give Int. 3C16 methyl 3,5-difluoro-4-(piperidin-4-yl)benzoate (12 g, HCl salt). Ints. 1AB8/3C16 (3.5 g/3.6 g) were stirred 16 h in DCE/DMSO/DIPEA (7:3:1, 112 mL). STAB (12 g) was added at 0° C. and the mixture was stirred 16 h at 0° C. to RT. The OL (water/DCM) was dried, concentrated, and purified by FC (hexane/EtOAc 9:1) to give Int. 3C15 methyl 4-(1-((4-bromo-1-methyl-1H-pyrrolo[2,3-b]pyridin-2-yl)methyl)piperidin-4-yl)-3,5-difluorobenzoate (4.0 g).

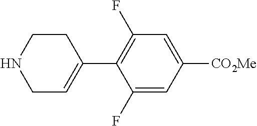 [SMILES: COC(=O)c1cc(F)c(C2=CCNCC2)c(F)c1]

Int. 3C25. methyl 3,5-difluoro-4-(1,2,3,6-tetrahydropyridin-4-yl)benzoate (16 g, HCl salt) was prepared similarly to Int. 3C16 from Int. 3C18 (20 g).

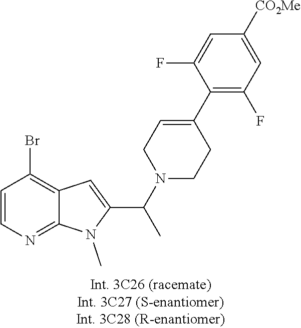 [SMILES: COC(=O)c1cc(F)c(C2=CCN(C(C)c3cc4c(Br)ccnc4n3C)CC2)c(F)c1]

Int. 1AF89 (9.0 g) was stirred 0.5 h in toluene/SOCl₂ (6:1, 174 mL) at 90° C. The residue after concentration, Cs₂CO₃ (43 g), KI (2.7 g), and Int. 3C25 (10 g) were stirred 16 h in ACN (250 mL) at 0° C. to RT under an inert atmosphere. The mixture was concentrated. The OL (water/EtOAc) was dried, concentrated, and purified by FC (PE/EtOAc 4:1) to give Int. 3C26 methyl 4-(1-(1-(4-bromo-1-methyl-1H-pyrrolo[2,3-b]pyridin-2-yl)ethyl)-1,2,3,6-tetrahydropyridin-4-yl)-3,5-difluorobenzoate (7.1 g). Int. 3C26 (0.85 g) was resolved by SFC on a SFC-150-008 instrument fitted with a Chiralpak-AD-H 150×25 mm 5 μm column operated at 30° C. and an eluent of 65% CO₂ and 35% MeOH (80 g/min) and a back pressure of 100 bar to give Int. 3C27 (0.30 g, first peak) and Int. 3C28 (0.30 g, second peak) methyl (S)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate and methyl (R)-4-(1-(1-(4-bromo-1-methyl-1H-pyrrolo[2,3-b]pyridin-2-yl)ethyl)-1,2,3,6-tetrahydropyridin-4-yl)-3,5-difluorobenzoate. The absolute configurations of these compounds were not determined.

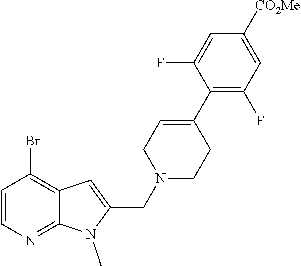 [SMILES: COC(=O)c1cc(F)c(C2=CCN(Cc3cc4c(Br)ccnc4n3C)CC2)c(F)c1]

Ints. 1AB8/3C25 (8.0 g/12.6 g) were stirred 16 h in DCE (80 mL) and DIPEA (35 mL) at 0° C. to RT under an inert atmosphere. STAB (28 g) was added in portions at 0° C. and stirring was continued for 12 h. The OL (water/DCM) was dried, concentrated, and purified by FC (PE/EtOAc 3:2) to give Int. 3C31 methyl 4-(1-((4-bromo-1-methyl-1H-pyrrolo[2,3-b]pyridin-2-yl)methyl)-1,2,3,6-tetrahydropyridin-4-yl)-3,5-difluorobenzoate (7.3 g).

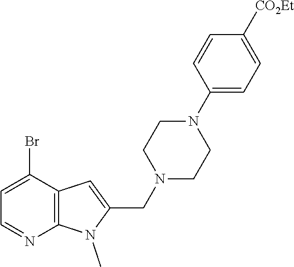 [SMILES: CCOC(=O)c1ccc(N2CCN(Cc3cc4c(Br)ccnc4n3C)CC2)cc1]

ZnCl₂ (1M in Et₂O, 31 mL) was added to a solution of Int. 1AB8 (2.7 g) and ethyl 4-(piperazin-1-yl)-benzoate (2.4 g) in THF (30 mL) at 0° C. and stirred 16 h at RT. NaCNBH₃ (3.4 g) was added and stirring continued for 16 h. The OL (water/EtOAc) was dried, concentrated, and purified by FC (PE/EtOAc 1:1) to give Int. 3C59 ethyl 4-(4-((4-bromo-1-methyl-1H-pyrrolo[2,3-b]pyridin-2-yl)methyl)-piperazin-1-yl)benzoate (2.2 g).

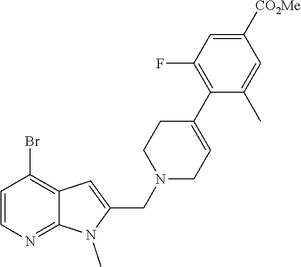 [SMILES: COC(=O)c1cc(C)c(C2=CCN(Cc3cc4c(Br)ccnc4n3C)CC2)c(F)c1]

DIPEA (7.3 mL) was added to a solution of Ints. 1AB8/1AF90 (2.0 g/2.9 g) at 0° C. and stirred in DCE (10 mL) for 16 h at RT under an inert atmosphere. STAB (5.2 g) was added at 0° C. and stirring continued for 2 h. The OL (water/EtOAc) was dried, concentrated, and purified by FC (hexane/EtOAc 3:2) to give Int. 3C73 methyl 4-(1-((4-bromo-1-methyl-1H-pyrrolo[2,3-b]pyridin-2-yl)methyl)-1,2,3,6-tetrahydropyridin-4-yl)-3-fluoro-5-methylbenzoate (1.3 g).

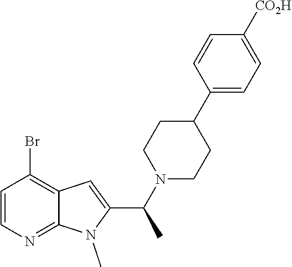 [SMILES: C[C@@H](c1cc2c(Br)ccnc2n1C)N1CCC(c2ccc(C(=O)O)cc2)CC1]

Int. 3E35 (1.7 g) and LiOH—H₂O (1.1 g) were stirred 3 h in THE/MeOH/H₂O (4:2:1, 25 mL) at 0° C. to RT and concentrated. The AL (water/Et₂O) was acidified with KHSO₄ to precipitate Int. 2C36 (S)-4-(1-(1-(4-bromo-1-methyl-1H-pyrrolo[2,3-b]pyridin-2-yl)ethyl)piperidin-4-yl)benzoic acid (1.5 g). The absolute configuration was determined for derivatives 1AF76 and 1AF76′.

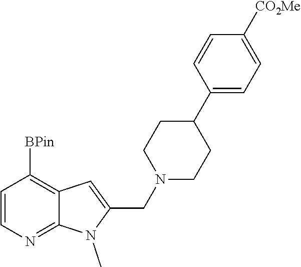

Int. 1AC5 (3.0 g), B₂Pin₂ (1.1 g), KOAc (2.0 g), and PdDPPFCl₂-DCM (50 mg) were degassed in dioxane (30 mL) and stirred ON at 90° C. The filtrate after filtration was concentrated and purified by FC (pentane/EtOAc 1:4) to give Int. 2H14 methyl 4-[1-[[1-methyl-4-(4,4,5,5-tetramethyl-1,3,2-dioxaborolan-2-yl)pyrrolo[2,3-b]pyridin-2-yl]methyl]-4-piperidyl]benzoate (1.7 g).

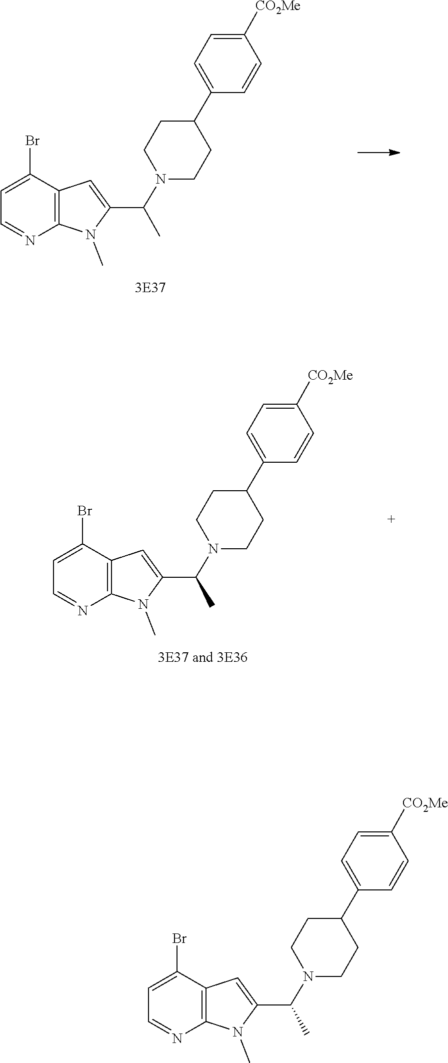 [SMILES: COC(=O)c1ccc(C2CCN(C(C)c3cc4c(Br)ccnc4n3C)CC2)cc1 ; COC(=O)c1ccc(C2CCN([C@@H](C)c3cc4c(Br)ccnc4n3C)CC2)cc1 ; COC(=O)c1ccc(C2CCN([C@H](C)c3cc4c(Br)ccnc4n3C)CC2)cc1]

Int. 1AF21 (1.0 g) was stirred 0.3 h in toluene/SOCl₂ (7.1:1, 22.8 mL) at 85-90° C. The residue after concentration, Cs₂CO₃ (3.8 g), KI (0.33 g), and methyl 4-(piperidin-4-yl)-benzoate (1.3 g, HCl salt) were stirred ON in ACN (25 mL) at 0° C. to RT. The mixture was concentrated to half volume and diluted with water to precipitate Int. 3E37 methyl 4-(1-(1-(4-bromo-1-methyl-1H-pyrrolo[2,3-b]pyridin-2-yl)ethyl)-piperidin-4-yl)benzoate (1.4 g). Int. 3E37 (7.4 g) was resolved by SFC using a Chiralpak IA 250X25 5 μm column operated at 30° C. using an eluent of 60% CO₂ and 40% MeOH at a flow rate of 90 g/min and a back pressure of 100 bar to give Int. 3E35 methyl (S)-4-(1-(1-(4-bromo-1-methyl-1H-pyrrolo[2,3-b]pyridin-2-yl)ethyl)piperidin-4-yl)benzoate (3.0 g, first eluting isomer) and Int. 3E36 methyl (R)-4-(1-(1-(4-bromo-1-methyl-1H-pyrrolo[2,3-b]pyridin-2-yl)ethyl)piperidin-4-yl)benzoate (3.0 g, second eluting isomer). The absolute configuration was determined for derivatives 1AF76 and 1AF76′.

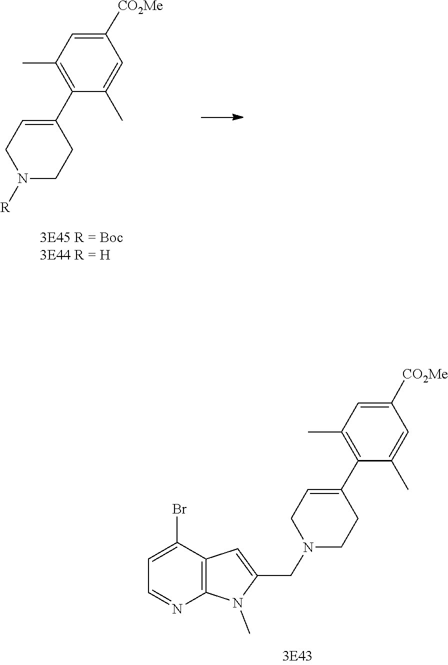

Methyl 4-bromo-3,5-dimethyl-benzoate (0.50 g), tert-butyl 4-(4,4,5,5-tetramethyl-1,3,2-dioxaborolan-2-yl)-3,6-dihydropyridine-1(2H)-carboxylate (0.70 g), Na₂CO₃ (0.26 g), and PdDPPFCl₂-DCM (84 mg) were degassed in dioxane/water (9:1; 8 mL) and stirred ON at 90° C. The OL (water/EtOAc) was concentrated and purified by FC (hexane/EtOAc 7:3) to give Int. 3E45 tert-butyl 4-(4-methoxycarbonyl-2,6-dimethyl-phenyl)-3,6-dihydro-2H-pyridine-1-carboxylate (0.55 g). This material was stirred 3 h in DCM/4M HCl in dioxane (5:4; 9 mL) at 0° C. to RT and concentrated to give Int. 3E44 methyl 3,5-dimethyl-4-(1,2,3,6-tetrahydro-pyridin-4-yl)benzoate (0.44 g, HCl salt). Ints. 1AB8/3E44 (0.23 g/0.30 g) were stirred 4 h in DCE/DIPEA (12.5:1, 5.4 mL). STAB (0.41 g) was added and stirring continued ON. The OL (water/EtOAc) was dried, concentrated, and purified by FC (hexane/EtOAc 7:3) to give Int. 3E43 methyl 4-[1-[(4-bromo-1-methyl-pyrrolo[2,3-b]pyridin-2-yl)methyl]-3,6-dihydro-2H-pyridin-4-yl]-3,5-dimethyl-benzoate (0.33 g).

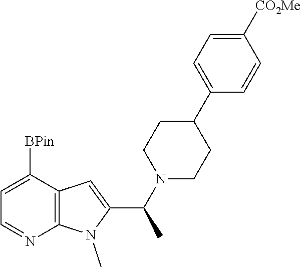

Int. 3E35 (2.0 g), B₂Pin₂ (1.7 g), PdDPPFCl₂-DCM (0.18 g), KOAc (0.9 g) were degassed in dioxane (15 mL) and stirred 5 h at 95-100° C., filtered, concentrated, and triturated in Et₂O to give Int. 3E170 methyl 4-[1-[(1S)-1-[1-methyl-4-(4,4,5,5-tetramethyl-1,3,2-dioxaborolan-2-yl)-pyrrolo[2,3-b]pyridin-2-yl]-ethyl]-4-piperidyl]benzoate (2.3 g).

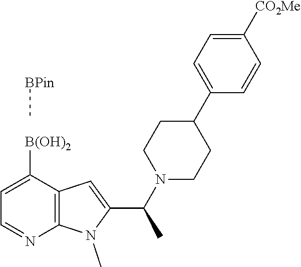

Int. 3E35 (0.50 g), B₂Pin₂ (0.41 g), PdDPPFCl₂-DCM (44 mg), KOAc (0.21 g) were degassed in dioxane (10 mL) and stirred 5 h at 95-100° C., filtered, concentrated, and triturated in Et₂O to give a mixture of Int. 3E170 and 3E177 (S)-(2-(1-(4-(4-(methoxycarbonyl)phenyl)piperidin-1-yl)-ethyl)-1-methyl-1H-pyrrolo[2,3-b]pyridin-4-yl)boronic acid and methyl (S)-4-(1-(1-(1-methyl-4-(4,4,5,5-tetramethyl-1,3,2-dioxaborolan-2-yl)-1H-pyrrolo[2,3-b]pyridin-2-yl)ethyl)piperidin-4-yl)benzoate (0.50 g).

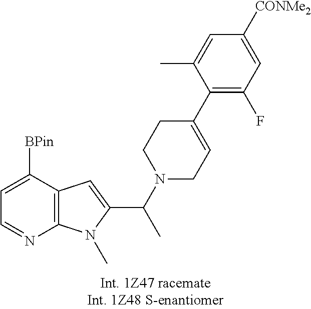

PdDPPFCl₂-DCM (40 mg), Int. 1Z69 (0.24 g), bis(pinacolato)diboron (0.15 g), and KOAc (0.14 g) were stirred ON in dioxane (5 mL) at 100° C. under an inert atmosphere. The mixture was concentrated and purified by FC (THF) to give Int. 1Z47 3-fluoro-N,N,5-trimethyl-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.27 g). Int. 1AF84′ (0.20 g), bis(pinacolato)diboron (0.20 g), PdDPPFCl₂-DCM (32 mg), and KOAc (0.12 g) were stirred 6 h in dioxane (10 mL) at 90° C. under an inert atmosphere. The mixture was filtered through celite, concentrated, dissolved in Et₂O, filtered, concentrated, washed with pentane, and dried to give Int. 1Z48 (S)-3-fluoro-5-methyl-N,N-bis(methyl-d₃)-4-(1-(1-(1-methyl-4-(4,4,5,5-tetramethyl-1,3,2-dioxaborolan-2-yl)-1H-pyrrolo[2,3-b]pyridin-2-yl)ethyl)-1,2,3,6-tetrahydropyridin-4-yl)benzamide (0.14 g).

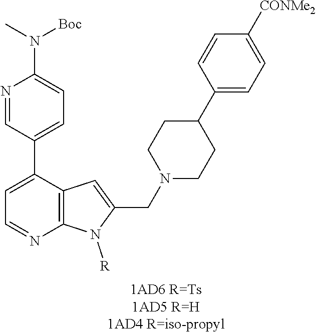

Int. 1AD7 (1.0 g), tert-butyl N-methyl-N-[5-(4,4,5,5-tetramethyl-1,3,2-dioxaborolan-2-yl)-2-pyridyl] (0.62 g), PdDPPFCl₂-DCM (0.21 g), and Na₂CO₃ (0.53 g) were degassed in DMF/water (10:1, 11 mL) and stirred at 100° C. ON and filtered. The OL (EtOAc/water) was washed with brine, dried, concentrated, and purified by FC (pentane/EtOAc 1:4) to give Int. 1AD6 tert-butyl N-[5-[2-[[4-[4-(dimethylcarbamoyl)-phenyl]-1-piperidyl]-methyl]-1-(p-tolylsulfonyl)pyrrolo[2,3-b]pyridin-4-yl]-2-pyridyl]-N-methyl-carbamate (0.5 g). Int. 1AD6 (1.0 g) was stirred ON in 0.1M TBAF in THF (23 mL) at 70° C. The OL (EtOAc/sat. aq. NaHCO₃) was washed with brine, dried, concentrated, and purified by FC (DCM/MeOH 9:1) to give int. IADS tert-butyl N-[5-[2-[[4-[4-(dimethyl-carbamoyl)phenyl]-1-piperidyl]methyl]-1H-pyrrolo[2,3-b]pyridin-4-yl]-2-pyridyl]-N-methyl-carbamate (0.4 g. Int. 1AD5 (0.10 g) and NaH (14 mg) were stirred 0.25 h in DMF (5 mL) at 0° C. 2-Bromopropane (0.17 mL) was added and stirring continued ON at 0° C. to RT. The OL (EtOAc/water) was washed with brine, dried, concentrated, and purified by FC (pentane/EtOAc 1:4) to give Int 1AD4 (60 mg).

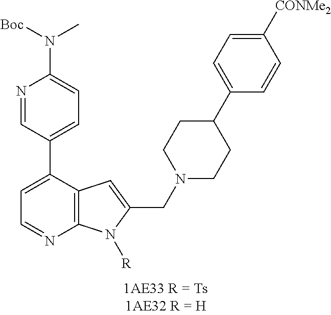

Int. 1AE34 (1.0 g), tert-butyl N-methyl-N-[5-(4,4,5,5-tetramethyl-1,3,2-dioxaborolan-2-yl)-2-pyridyl]-carbamate (0.62 g), PdDPPFCl₂-DCM (0.21 g), and Na₂CO₃ (0.53 g) were degassed in DMF/water (9:1, 20 mL) and stirred ON at 100° C. and filtered. The OL. (water/EtOAc) was washed with brine, dried, concentrated, and purified by FC (EtOAc/hexane 4:1) to give int. 1AE33 tert-butyl N-[5-[2-[[4-[4-(dimethylcarbamoyl)-phenyl]-1-piperidyl]-methyl]-1-(p-tolylsulfonyl)-pyrrolo[2,3-b]pyridin-4-yl]-2-pyridyl]-N-methyl-carbamate (0.50 g). Int. 1AE33 (0.45 g) was stirred 3 h in 0.15 M TBAF in THF (16.3 mL) at 70° C. The OL (EtOAc/sat. aq. NaHCO₃.) was dried, concentrated, and purified by FC (DCM/MeOH 9:1) to give Int. 1AE32 tert-butyl N-[5-[2-[[4-[4-(dimethylcarbamoyl)-phenyl]-1-piperidyl]methyl]-1H-pyrrolo[2,3-b]pyridin-4-yl]-2-pyridyl]-N-methyl-carbamate (0.25 g).

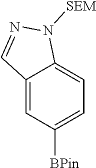

### Int. 1AF16. 5-(4,4,5,5-Tetramethyl-1,3,2-dioxaborolan-2-yl)-1-((2-(trimethylsilyl)ethoxy)methyl)-1H-indazole

SEM-Cl (26.9 mL) was added slowly to a solution of 5-bromo-1H-indazole (20 g) in DCM (0.5 L) and 50% aq. KOH (34.2 g) at 0° C. and stirring continued 5 h at 0° C. to RT. The OL (water/DCM) was dried, concentrated, and purified by FC (pentane/EtOAc 95:5) to give 2-[(5-bromoindazol-1-yl)methoxy]ethyl-trimethyl-silane (17 g). This material, B₂Pin₂ (1.58 g), KOAc (1.52 g), and PdDPPFCl₂-DCM (0.84 g) were degassed in dioxane (300 mL) and stirred ON at 100° C. The mixture was filtered, concentrated, and purified by FC (pentane/EtOAc 95:5) to give Int. 1AF16 (16 g).

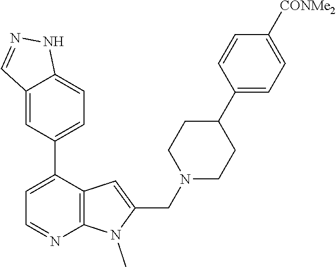

Int. 1AF17 (0.10 g), 1-(tetrahydro-2H-pyran-2-yl)-5-(4,4,5,5-tetramethyl-1,3,2-dioxaborolan-2-yl)-1H-indazole (91 mg), and PdDPPFCl₂-DCM (17 mg) were degassed in DMF/10% aq. Na₂CO₃ (6.7:1, 3.5 mL) and stirred ON at 90° C. The mixture was filtered, concentrated, and purified by FC (hexane/EtOAc 1:4) give Int. 1AF18 4-[1-[1-[4-(6-Amino-3-pyridyl)-1-methyl-pyrrolo[2,3-b]pyridin-2-yl]ethyl]-4-piperidyl]-N,N-dimethyl-benzamide (0.15 g).

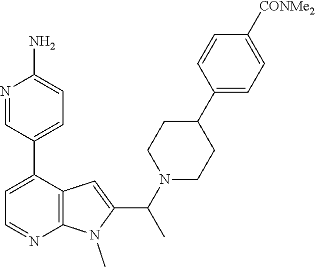

Int. 1AF17 (75 mg), 5-(4,4,5,5-tetramethyl-1,3,2-dioxaborolan-2-yl)pyridin-2-amine (39 mg), Na₂CO₃ (39 mg), and PdCPPFCl₂-DCM (7 mg) were degassed in DMF/water (4:1, 2.5 mL) and stirred ON at 90° C. and filtered. The OL (EtOAc/water) was concentrated and purified by FC (hexane/EtOAc 1:4) to give Int. 1AF19 4-[1-[1-[4-(6-Amino-3-pyridyl)-1-methyl-pyrrolo[2,3-b]pyridin-2-yl]ethyl]-4-piperidyl]-N,N-dimethyl-benzamide (40 mg).

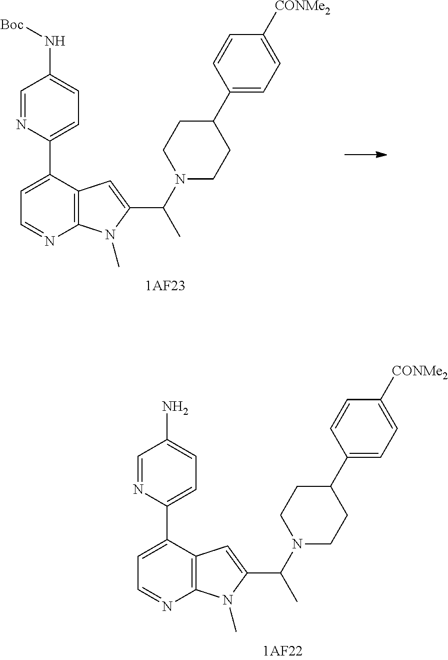

Int. 1AF24 (0.40 g), tert-butyl N-(6-bromo-3-pyridyl)carbamate (0.12 g), PdDPPFCl₂-DCM (38 mg), and K₂CO₃ (0.19 g) were degassed in dioxane/water (4:1, 20 mL) and stirred ON at 100° C. and filtered. The OL (water/EtOAc) was dried, concentrated, and purified by FC (hexane/EtOAc 1:4) to give Int. 1AF23 (0.20 g). Int. 1AF23 (0.50 g) was stirred ON in DCM/TFA (20:1, 21 mL) at 0° C. to RT, concentrated, and HPLC-purified to give Int. 1AF22 4-(1-(1-(4-(5-Aminopyridin-2-yl)-1-methyl-1H-pyrrolo[2,3-b]pyridin-2-yl)ethyl)piperidin-4-yl)-N,N-dimethylbenzamide (80 mg).

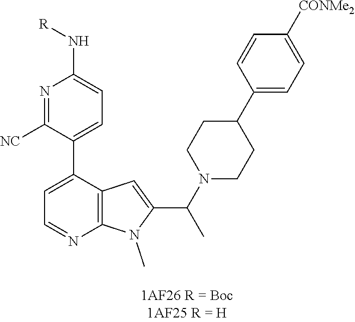

Int. 1AF27 (0.12 g), tert-butyl N-(5-bromo-6-cyano-2-pyridyl)carbamate (69 mg), Na₂CO₃ (39 mg), and PdCPPFCl₂-DCM (7 mg) were degassed in DMF/water (4:1, 2.5 mL) and stirred at 90° C. ON and filtered. The OL (EtOAc/water) was concentrated and purified by FC (hexane/EtOAc 1:4) to give Int. 1AF26 tert-butyl (6-cyano-5-(2-(1-(4-(4-(dimethylcarbamoyl)-phenyl)piperidin-1-yl)ethyl)-1-methyl-1H-pyrrolo[2,3-b]pyridin-4-yl)pyridin-2-yl)carbamate (0.12 g). This material was stirred ON in DCM/TFA (10:1, 11 mL) at 0° C. to RT, concentrated, and HPLC-purified to give Int. 1AF25 4-(1-(1-(4-(6-Amino-2-cyanopyridin-3-yl)-1-methyl-1H-pyrrolo[2,3-b]pyridin-2-yl)ethyl)piperidin-4-yl)-N,N-dimethylbenzamide (23 mg).

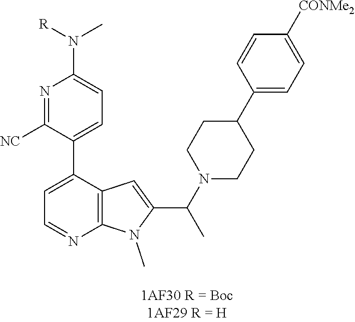

Int. 1AF30 tert-butyl (6-cyano-5-(2-(1-(4-(4-(dimethylcarbamoyl)phenyl)piperidin-1-yl)ethyl)-1-methyl-1H-pyrrolo[2,3-b]pyridin-4-yl)pyridin-2-yl)(methyl)carbamat (0.12 g) was prepared from Int. 1AF27 (0.20 g) and tert-butyl N-(5-bromo-6-cyano-2-pyridyl)-N-methyl-carbamate (0.22 g) similarly to Int. 1AF26. Int. 1AF29 4-(1-(1-(4-(2-Cyano-6-(methylamino)pyridin-3-yl)-1-methyl-1H-pyrrolo[2,3-b]pyridin-2-yl)ethyl)piperidin-4-yl)-N,N-dimethylbenzamide (30 mg) was prepared similarly to Int. 1AF25 from Int. 1AF30 (0.10 g).

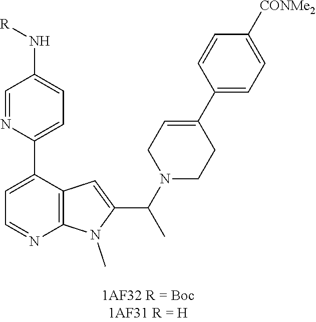

Int. 1AF32 tert-butyl (6-(2-(1-(4-(4-(dimethylcarbamoyl)phenyl)-3,6-dihydropyridin-1(2H)-yl)ethyl)-1-methyl-1H-pyrrolo[2,3-b]pyridin-4-yl)pyridin-3-yl)carbamate (0.28 g) was prepared similarly to Int. 1AF30 from Int. 1AF33 (0.60 g) and tert-butyl N-(6-bromo-3-pyridyl)-carbamate (0.23 g). Int. 1AF31 4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N-dimethylbenzamide (60 mg) was prepared similarly to Int. 1AF25 from Int. 1AF32 (0.18 g).

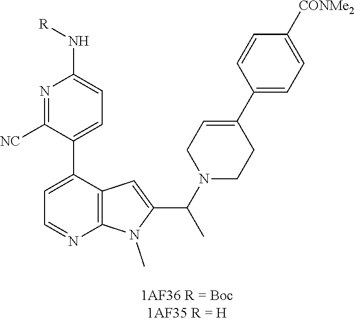

Int. 1AF35. 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide (40 mg) was prepared similarly to Int. 1AF25 from Int. 1AF36 (0.12 g). 1AF36 tert-butyl (6-cyano-5-(2-(1-(4-(4-(dimethylcarbamoyl)phenyl)-3,6-dihydropyridin-1(2H)-yl)ethyl)-1-methyl-1H-pyrrolo[2,3-b]pyridin-4-yl)pyridin-2-yl)carbamate (0.14 g) was prepared similarly to Int. 1AF32 from Int. 1AF33 (0.25 g) and tert-butyl N-(5-bromo-6-cyano-2-pyridyl)carbamate (0.13 g).

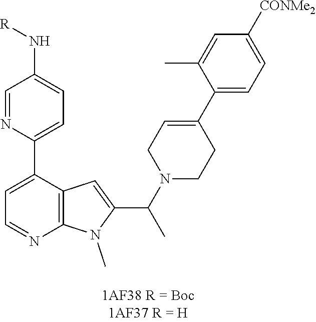

1AF38 tert-butyl N-[6-[2-[1-[4-[4-(dimethylcarbamoyl)-2-methyl-phenyl]-3,6-dihydro-2H-pyridin-1-yl]ethyl]-1-methyl-pyrrolo[2,3-b]pyridin-4-yl]-3-pyridyl]carbamate (50 mg) was prepared similarly to Int. 1AF25 from Int. 1AF39 (0.10 g). Int. 1AF37 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide (48 mg) was prepared similarly to Int. 1AF25 from Int. 1AF38 (0.15 g).

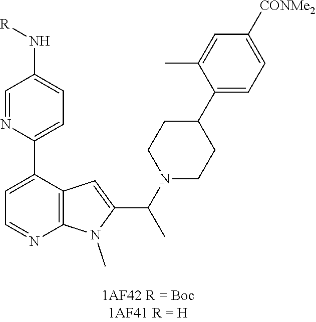

Int. 1AF42 tert-butyl N-[6-[2-[1-[4-[4-(dimethyl-carbamoyl)phenyl]-1-piperidyl]ethyl]-1-methyl-pyrrolo[2,3-b]pyridin-4-yl]-3-pyridyl]carbamate (0.20 g) was prepared similarly to Int. 1AF23 from Int. 1AF43 (0.40 g) and tert-butyl N-(6-bromo-3-pyridyl)carbamate (0.15 g). Int. 1AF41 4-(1-(1-(4-(5-Aminopyridin-2-yl)-1-methyl-1H-pyrrolo[2,3-b]pyridin-2-yl)ethyl)piperidin-4-yl)-N,N,3-trimethylbenzamide (63 mg) was prepared similarly to Int. 1AF25 from Int. 1AF42 (0.20 g).

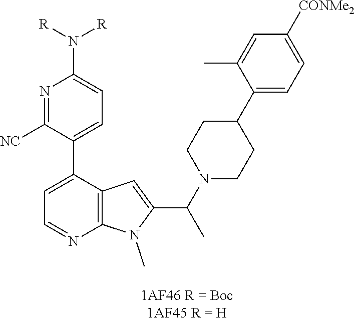

Int. 1AF46 tert-butyl (tert-butoxycarbonyl)(6-cyano-5-(2-(1-(4-(4-(dimethylcarbamoyl)-2-methyl-phenyl)-piperidin-1-yl)ethyl)-1-methyl-1H-pyrrolo[2,3-b]pyridin-4-yl)pyridin-2-yl)carbamate (50 mg) was prepared similarly to Int. 1AF42 from Int. 1AF43 (0.35 g) and di tert-butyl N-(5-bromo-6-cyano-2-pyridyl)carbamate (0.47 g). Int. 1AF45 4-(1-(1-(4-(6-Amino-2-cyanopyridin-3-yl)-1-methyl-1H-pyrrolo[2,3-b]pyridin-2-yl)ethyl)piperidin-4-yl)-N,N,3-trimethylbenzamide (41 mg) was prepared similarly to Int. 1AF25 from Int. 1AF46 (0.14 g).

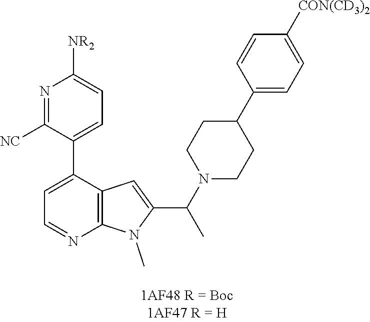

Int. 1AF48 tert-butyl N-[5-[2-[1-[4-[4-[bis(trideuteriomethyl)-carbamoyl]-phenyl]-1-piperidyl]ethyl]-1-methyl-pyrrolo[2,3-b]pyridin-4-yl]-6-cyano-2-pyridyl]-N-tert-butoxycarbonyl-carbamate (0.41 g) was prepared similarly to Int. 1AF26 from Int. 1AF49 (0.35 g) and tert-butyl N-(5-bromo-6-cyano-2-pyridyl)-N-tert-butoxy-carbonyl-carbamate (0.41 g). Int. 1AF47 4-(1-(1-(4-(6-Amino-2-cyanopyridin-3-yl)-1-methyl-1H-pyrrolo[2,3-b]pyridin-2-yl)ethyl)piperidin-4-yl)-N,N-bis(methyl-d₃)benzamide (95 mg) was prepared similarly to Int. 1AF25 from Int. 1AF48 (0.35 g).

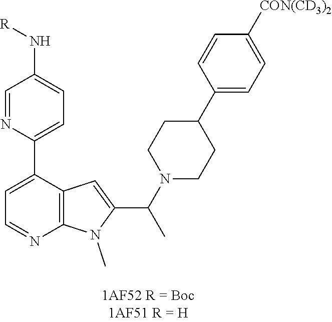

Int. 1AF52 tert-butyl N-[6-[2-[1-[4-[4-[bis(trideuteriomethyl)carbamoyl]phenyl]-1-piperidyl]ethyl]-1-methyl-pyrrolo[2,3-b]pyridin-4-yl]-3-pyridyl]carbamate (0.38 g) was prepared similarly to Int. 1AF42 from Int. 1AF50 (0.35 g) and tert-butyl N-(6-bromo-3-pyridyl)carbamate (0.30 g). Int. 1AF51 4-(1-(1-(4-(5-Aminopyridin-2-yl)-1-methyl-1H-pyrrolo[2,3-b]pyridin-2-yl)ethyl)piperidin-4-yl)-N,N-bis(methyl-d₃)benzamide (92 mg) was prepared similarly to Int. 1AF25 from Int. 1AF52 (0.38 g).

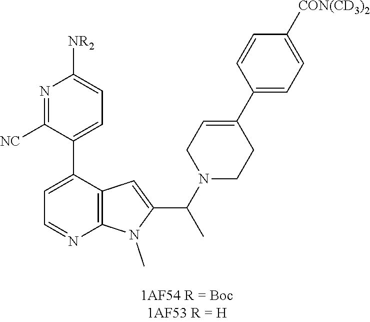

Int. 1AF54 tert-butyl N-[5-[2-[1-[4-[4-[bis(trideuteriomethyl)carbamoyl]phenyl]-3,6-dihydro-2H-pyridin-1-yl]ethyl]-1-methyl-pyrrolo[2,3-b]pyridin-4-yl]-6-cyano-2-pyridyl]-N-tert-butoxy-carbonyl-carbamate (0.45 g) was prepared similarly to from Int. 1AF48 from Int. 1AF55 (0.35 g) and tert-butyl N-(5-bromo-6-cyano-2-pyridyl)-N-tert-butoxycarbonyl-carbamate (0.35 g). Int. 1AF53 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)-benzamide (60 mg) was prepared similarly to Int. 1AF25 from Int. 1AF54 (0.20 g).

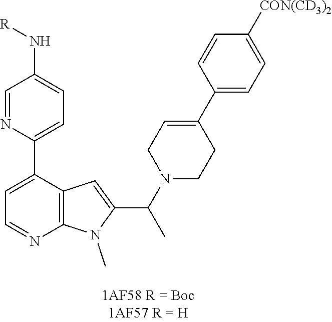

Int. 1AF58 tert-butyl (6-(2-(1-(4-(4-(bis(methyl-d₃)carbamoyl)phenyl)-3,6-dihydropyridin-1(2H)-yl)ethyl)-1-methyl-1H-pyrrolo[2,3-b]pyridin-4-yl)pyridin-3-yl)carbamate (0.20 g) was prepared similarly to like Int. 1AF42 from Int. 1AF55 (0.15 g) and tert-butyl N-(6-bromo-3-pyridyl)carbamate (0.22 g). Int. 1AF57 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide (80 mg) was prepared similarly to Int. 1AF25 obtained from Int. 1AF58 (0.15 g).

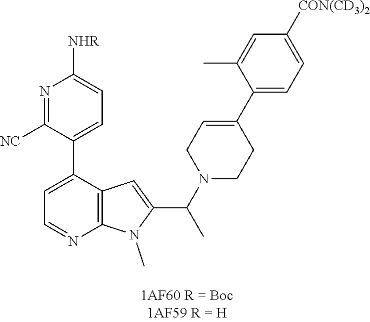

Int. 1AF60 tert-butyl (5-(2-(1-(4-(4-(bis(methyl-d₃)carbamoyl)-2-methyl-phenyl)-3,6-dihydropyridin-1(2H)-yl)ethyl)-1-methyl-1H-pyrrolo[2,3-b]pyridin-4-yl)-6-cyanopyridin-2-yl)-carbamate (0.25 g) was prepared similarly to Int. 1AF32 from Int. 1AF61 (0.40 g) and tert-butyl N-(5-bromo-6-cyano-2-pyridyl)carbamate (0.34 g). Int. 1AF59 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide (48 mg) was prepared similarly to Int. 1AF25 from Int. 1AF60 (0.20 g).

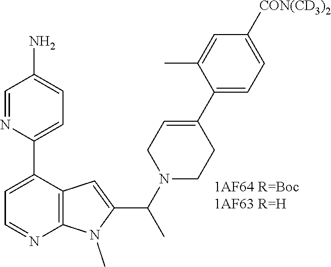

Int. 1AF64 tert-butyl (6-(2-(1-(4-(4-(bis(methyl-d₃)carbamoyl)-2-methylphenyl)-3,6-dihydropyridin-1(2H)-yl)-ethyl)-1-methyl-1H-pyrrolo[2,3-b]pyridin-4-yl)pyridin-3-yl)carbamate (0.22 g) was prepared similarly to Int. 1AF42 from Int. 1AF61 (0.30 g) and tert-butyl N-(6-bromo-3-pyridyl)carbamate (0.16 g). Int. 1AF63 4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide (38 mg) was prepared similarly to Int. 1AF25 from Int. 1AF64 (0.24 g).

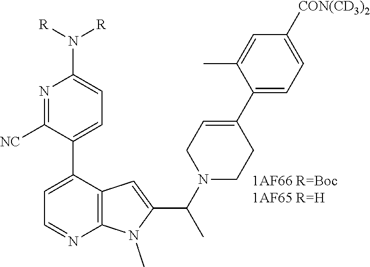

Int. 1AF66 tert-butyl (tert-butoxycarbonyl)(6-cyano-5-(2-(1-(4-(4-(dimethylcarbamoyl)-2-methylphenyl)-3,6-dihydropyridin-1(2H)-yl)ethyl)-1-methyl-1H-pyrrolo[2,3-b]pyridin-4-yl)pyridin-2-yl)carbamate (0.17 g) was prepared similarly to Int. 1AF42 from Int. 1AF39 (0.20 g) and tert-butyl N-(5-bromo-6-cyano-2-pyridyl)-N-tert-butoxycarbonyl-carbamate (0.21 g). Int. 1AF65 4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N,3-trimethylbenzamide (24 mg) was prepared similarly to Int. 1AF25 from Int. 1AF66 (0.17 g).

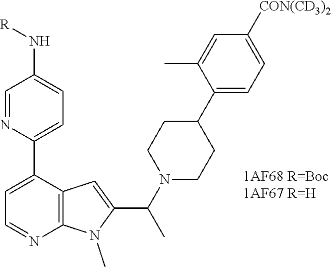

Int. 1AF68 tert-butyl (6-(2-(1-(4-(4-(bis(methyl-d3)carbamoyl)-2-methylphenyl)piperidin-1-yl)ethyl)-1-methyl-1H-pyrrolo[2,3-b]pyridin-4-yl)pyridin-3-yl)carbamate (0.25 g) was prepared similarly to Int. 1AF42 from Int. 1AF49 (0.30 g) and tert-butyl N-(6-bromo-3-pyridyl)carbamate (0.37 g). Int. 1AF67 4-(1-(1-(4-(5-Aminopyridin-2-yl)-1-methyl-1H-pyrrolo[2,3-b]pyridin-2-yl)ethyl)piperidin-4-yl)-3-methyl-N,N-bis(methyl-d₃)benzamide (55 mg) was prepared similarly to Int. 1AF25 from Int. 1AF68 (0.25 g).

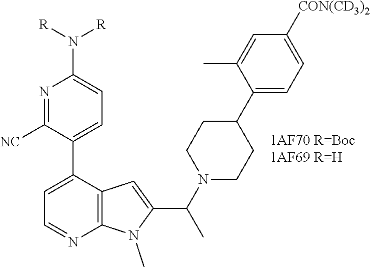

Int. 1AF70 tert-butyl (5-(2-(1-(4-(4-(bis(methyl-d₃)carbamoyl)-2-methylphenyl)-piperidin-1-yl)ethyl)-1-methyl-1H-pyrrolo[2,3-b]pyridin-4-yl)-6-cyanopyridin-2-yl)(tert-butoxy-carbonyl)-carbamate (0.28 g) was prepared similarly to Int. 1AF42 from Int. 1AF71 (0.35 g) and tert-butyl N-(5-bromo-6-cyano-2-pyridyl)-N-tert-butoxycarbonyl-carbamate (0.46 g). Int. 1AF69 4-(1-(1-(4-(6-Amino-2-cyanopyridin-3-yl)-1-methyl-1H-pyrrolo[2,3-b]-pyridin-2-yl)ethyl)piperidin-4-yl)-3-methyl-N,N-bis(methyl-d₃)benzamide (57 mg) was prepared similarly to Int. 1AF25 from Int. 1AF70 (0.23 g).

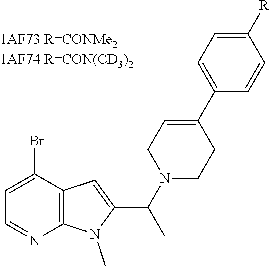

Int. 1AF21 (1.20 g) was stirred 1 h in toluene/SOCl₂ (5.9:1, 23.5 mL) at 0° C. to 110° C. and concentrated. The residue, Cs₂CO₃ (4.60 g), KI (0.39 g), and Int. 1AE7 (1.30 g) were stirred ON in ACN (30 mL) at 0° C. to RT. The OL (water/EtOAc) was washed with brine, dried, concentrated, and purified by FC (hexane/EtOAc 9:1 to 85:15) to give Int. 1AF73 4-[1-[1-(4-bromo-1-methyl-pyrrolo[2,3-b]pyridin-2-yl)ethyl]-3,6-dihydro-2H-pyridin-4-yl]-N,N-dimethyl-benzamide (1.0 g). Int. 1AF74 4-(1-(1-(4-bromo-1-methyl-1H-pyrrolo[2,3-b]pyridin-2-yl)ethyl)-1,2,3,6-tetrahydropyridin-4-yl)-N,N-bis(methyl-d₃)benzamide (0.60 g) was prepared similarly from Ints. 1AE7′/1AF21 (0.69 g/0.50 g).

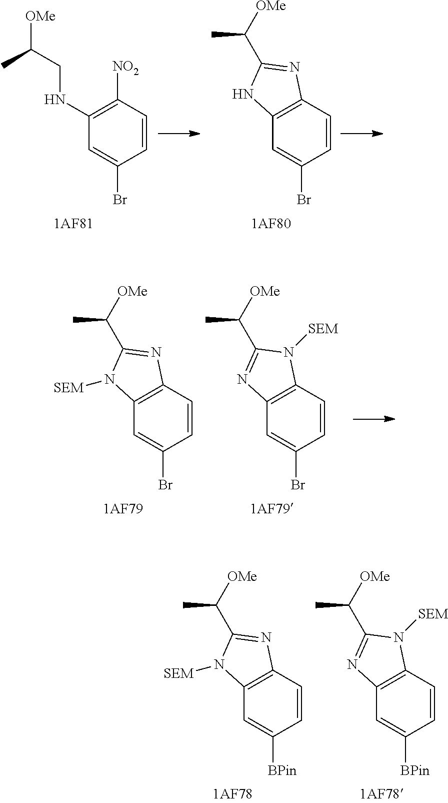

(R)-2-methoxypropanoic acid (2.11 g) was stirred 1 h in pyridine/POCl₃ (17.4:1, 42.6 mL) at 0° C. 4-Bromo-2-nitroaniline (4.0 g) was added and stirring continued ON at 0° C. to RT. The mixture was diluted with aq. HCl to precipitate Int. 1AF81 (R)—N-(4-bromo-2-nitro-phenyl)-2-methoxy-propan-amide (4.0 g). This material and iron powder (2.21 g) were stirred 4 h in AcOH/EtOH (1.8:1, 55 mL) at 65° C. and filtered. The OL (EtOAc/water) was dried, concentrated, and triturated in Et₂O to give Int. 1AF80 5-bromo-2-[(R)-1-methoxyethyl]-1H-benz-imidazole (2.50 g). This material and NaH (0.35 g) were stirred 0.5 h in DMF (5 mL) at 0° C. SEM-Cl (2.0 mL) was added and stirring continued 3 h at 0° C. to RT. The OL (water/EtOAc) was dried, concentrated, and purified by FC (hexane/EtOAc 4:1) to give Ints. 1AF79/1AF79′ (R)-6-bromo-2-(1-methoxy-ethyl)-1-((2-(trimethylsilyl)-ethoxy)methyl)-1H-benzo[d]imidazole and (R)-5-bromo-2-(1-methoxyethyl)-1-((2-(trimethylsilyl)ethoxy)-methyl)-1H-benzo[d]-imidazole (2.0 g). 1.5 g of this mixture, KOAc (1.1 g), 4,4,4′,4′,5,5,5′,5′-octamethyl-2,2′-bi(1,3,2-dioxa-borolane) (2.0 g), and PdDPPFCl₂-DCM (0.32 g) were degassed in dioxane (5 mL) and stirred at 100° C. ON. The OL (water/EtOAc) was dried, concentrated, and purified by FC (hexane/EtOAc 55:45) to give a mixture of Ints. 1AF78 and 1AF78′ (R)-2-(1-methoxyethyl)-6-(4,4,5,5-tetramethyl-1,3,2-dioxaborolan-2-yl)-1-((2-(trimethylsilyl)ethoxy)methyl)-1H-benzo[d]imidazole and (R)-2-(1-methoxyethyl)-5-(4,4,5,5-tetramethyl-1,3,2-dioxaborolan-2-yl)-1-((2-(trimethylsilyl)ethoxy)methyl)-1H-benzo[d]imidazole (1.4 g).

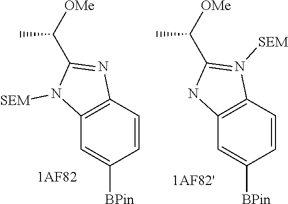

Ints. 1AF82/1AF82′ 2-((S)-1-methoxyethyl)-6-(4,4,5-trimethyl-1,3,2-dioxaborolan-2-yl)-1-((2-(trimethylsilyl)ethoxy)methyl)-1H-benzo[d]imidazole and (S)-2-(1-methoxyethyl)-5-(4,4,5,5-tetramethyl-1,3,2-dioxaborolan-2-yl)-1-((2-(trimethylsilyl)ethoxy)methyl)-1H-benzo[d]imidazole were prepared similarly to Ints. 1AF78/1AF78′ from (R)-2-methoxypropanoic acid.

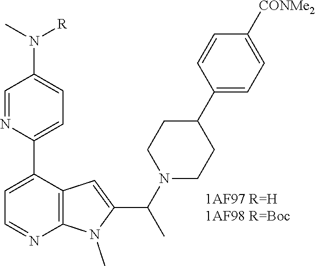

Int. 1AF24 (0.50 g), tert-butyl (6-bromopyridin-3-yl)(methyl)carbamate (0.26 g), PdDPPFCl₂-DCM (56 mg), and K₂CO₃ (0.29 g) were degassed in dioxane/water (4:1, 15 mL) and stirred ON at 100° C. The mixture was filtered. The filtrate was diluted with EtOAc and washed with water. The OL was dried, concentrated, and purified by FC (hexane/EtOAc 1:4) to give Int. 1AF98 tert-butyl N-[6-[2-[1-[4-[4-(dimethylcarbamoyl)phenyl]-1-piperidyl]ethyl]-1-methyl-pyrrolo[2,3-b]pyridin-4-yl]-3-pyridyl]-N-methyl-carbamate (0.16 g). Int. 1AF98 (0.15 g) was stirred ON in DCM/TFA (8:1, 9 mL) at 0° C. to RT. The mixture was concentrated and triturated with pentane to give a solid. This material was HPLC-purified to give Int. 1AF97 N,N-dimethyl-4-(1-(1-(1-methyl-4-(5-(methylamino)pyridin-2-yl)-1H-pyrrolo[2,3-b]pyridin-2-yl)ethyl)piperidin-4-yl)benzamide (22 mg).

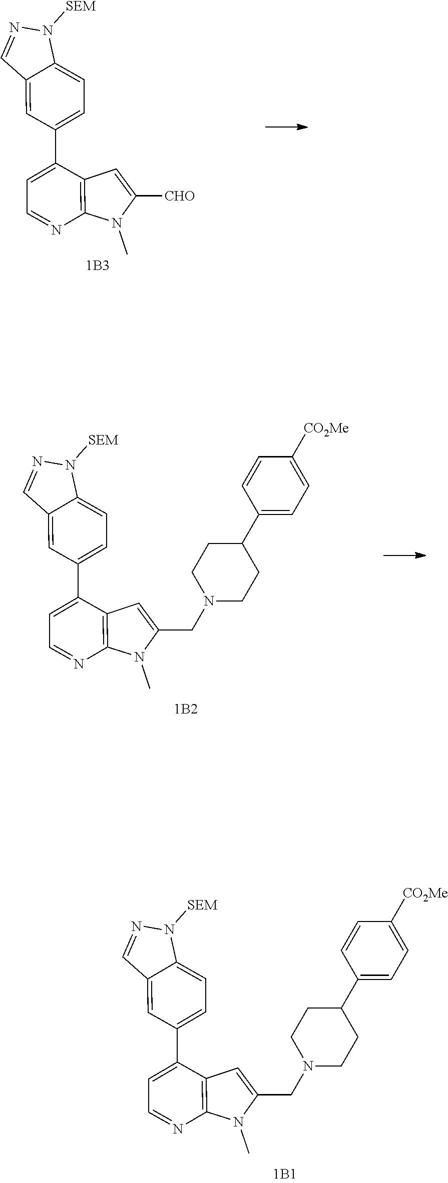 [SMILES: Cn1c(C=O)cc2c(-c3ccc4c(cnn4COCC[Si](C)(C)C)c3)ccnc21 ; COC(=O)c1ccc(C2CCN(Cc3cc4c(-c5ccc6c(cnn6COCC[Si](C)(C)C)c5)ccnc4n3C)CC2)cc1 ; COC(=O)c1ccc(C2CCN(Cc3cc4c(-c5ccc6c(cnn6COCC[Si](C)(C)C)c5)ccnc4n3C)CC2)cc1]

PdDPPFCl₂-DCM (0.34 g), Int. 1AB8 (2.0 g), trimethyl-[2-[[5-(4,4,5,5-tetramethyl-1,3,2-dioxaborolan-2-y)-indazol-1-yl]methoxy]ethyl]silane (4.7 g), and NaHCO₃ (2.2 g) were degassed in dioxane/water (5:1, 24 mL) and stirred ON at 110° C. The OL (water/EtOAc) was washed with brine, dried, concentrated, and purified by FC (hexane/EtOAc 1:1) to give Int. 1B3 1-methyl-4-(1-((2-(trimethylsilyl)ethoxy)-methyl-1H-indazol-5-yl)-1H-pyrrolo[2,3-b]pyridine-2-carbaldehyde (2.2 g). N,N-Dimethyl-4-(4-piperidinyl)-benzamide (0.58 g, HCl salt), Int. 1B3 (0.62 g), and 4A MS were stirred ON in DCM/AcOH (5:1, 6 mL), STAB (0.65 g) was added and stirring continued 2b. The mixture was filtered, concentrated, and purified by FC (heptane to EtOAc) to give Int. 1B2 methyl 4-(1-((1-methyl-4-(1-((2-(tri-methylsilyl)ethoxy)meth)-1H-indazol-5-yl)-1H-pyrrolo[2,3-b]pyridin-2-y)methy)-piperidin-4-yl)-benzoate (031 g). Int. 1B2 (88 mg) and LiOH (10 mg) were stirred ON in MeOH/water (2:1, 1 mL) at 50° C. The mixture was partially concentrated. The OL (aq. citric acid/EtOAc) was washed with brine, dried, and concentrated to give Int. 1B1 4-(1-((1-Methy-4-(1-((2-(trimethylsilyl)ethoxy)methyl)-1H-indazol-5-yl)-1H-pyrrolo[2,3-b]pyridin-2-yl)methyl)piperidin-4-y)benzoic acid (83 mg).

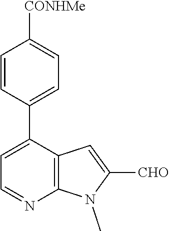

Int. 1AB8 (0.15 g), (4-(methylcarbamoyl)phenyl)boronic acid (0.17 g), PdDPPFCl₂-DCM (51 mg), and NaHCO₃ (0.16 g) were degassed in dioxane/water (2.4:1, 4.4 mL) and stirred 0.5 h at 100° C. The OL (water/EtOAc) was washed with brine, dried, concentrated, and purified by FC (DCM/MeOH 1:0 to 95:5) to give Int. 1J2 4-(2-formyl-1-methyl-1H-pyrrolo[2,3-b]pyridin-4-yl)-N-methylbenzamide (64 mg).

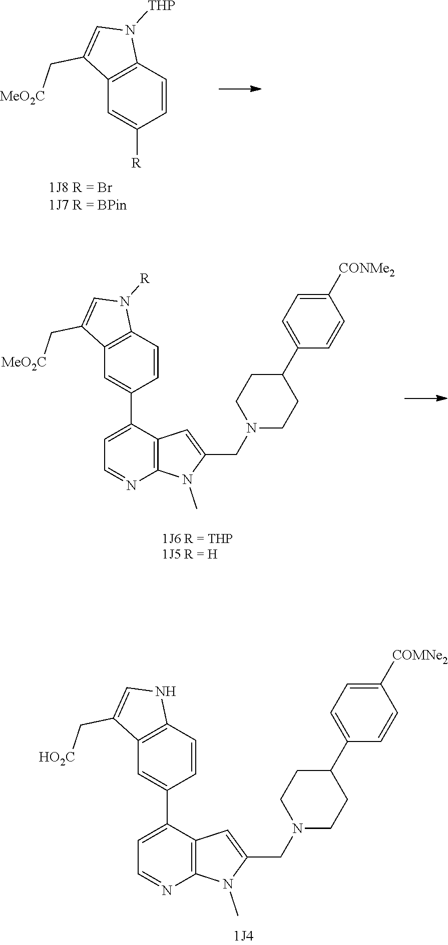

Methyl 2-(5-bromo-1H-indol-3-yl)acetate (30 g), 3,4-dihydro-2H-pyran (14.1 g), and TsOH-H₂O (4.26 g) were stirred in DCM (0.3 L) ON. The OL (DCM/water) was dried, concentrated, and purified by FC (pentane/EtOAc 1:0 to 85:15) to give Int. 1J8 methyl 2-(5-bromo-1-tetrahydropyran-2-yl-indol-3-yl)acetate (18 g). 5.0 g of this material, B₂Pin₂ (4.3 g), KOAc (2.8 g), and PdDPPFCl₂-DCM (0.52 g) were degassed in dioxane (50 mL) and stirred ON at 110° C. The mixture was filtered, concentrated, and purified by FC (pentane/EtOAc 1:0 to 7:3) to give Int. 1J7 methyl 2-(1-(tetrahydro-2H-pyran-2-yl)-5-(4,4,5,5-tetramethyl-1,3,2-dioxaborolan-2-yl)-1H-indol-3-yl)acetate (4.8 g). Ints. 1J7/1AB1 (4.5 g/3.0 g), NaHCO₃ (1.66 g), and PdDPPFCl₂-DCM (0.24 g) were degassed in dioxane/water (6.7:1, 23 mL) and stirred ON at 100° C. The mixture was filtered, concentrated, and purified by FC (DCM/MeOH 97:3 to 95:5) to give Int. 1J6 methyl 2-(5-(2-((4-(4-(dimethyl-carbamoyl)phenyl)-piperidin-1-yl)methyl)-1-methyl-1H-pyrrolo[2,3-b]pyridin-4-yl)-1-(tetrahydro-2H-pyran-2-yl)-1H-indol-3-yl)acetate (3.0 g). 15 mg of this material was stirred 3 h in DCM/TFA (1:1, 2 mL), concentrated, and HPLC-purified to give Int. 1J5 methyl 2-(5-(2-((4-(4-(dimethylcarbamoyl)-phenyl)piperidin-1-yl)methyl)-1-methyl-1H-pyrrolo[2,3-b]pyridin-4-yl)-1H-indol-3-yl)acetate (5 mg). This material was stirred 1 h in MeOH/1M aq. LiOH (1:1, 2 mL), neutralized, and HPLC-purified to give Int. 1J4 2-(5-(2-((4-(4-(dimethylcarbamoyl)phenyl)piperidin-1-yl)methyl)-1-methyl-1H-pyrrolo[2,3-b]pyridin-4-yl)-1H-indol-3-yl)acetic acid (3.9 mg).

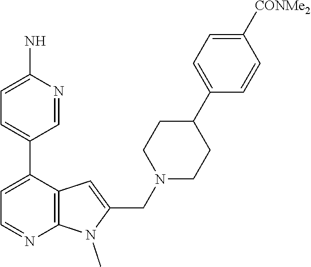

Int. 1AC4 (0.30 g, HCl salt), N-methyl-5-(4,4,5,5-tetramethyl-1,3,2-dioxaborolan-2-yl)pyridin-2-amine (0.17 g), PdDPPFCl₂-DCM (53 mg), K₂CO₃ (0.47 g) were degassed in DMF/water (8:1, 4.2 mL) and stirred at 100° C. ON. The reaction was repeated at the same scale. The combined residues after concentration of the filtrates were purified by HPLC to give Int. 1L1 4-[1-[[1-Methyl-4-[6-(methylamino)-3-pyridyl]pyrrolo[2,3-b]pyridin-2-yl]methyl]-4-piperidyl]benzoic acid (0.29 g).

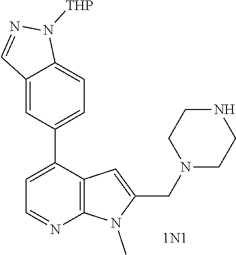

Int. 1N2 (0.15 g), K₂CO₃ (34 mg), Int. 1AF16 (0.3 g), and PdDPPFCl₂-DCM (7 mg) were degassed in DMF/water (10:1, 2.2 mL) and stirred 4 h at 90° C. and filtered. The OL (water/EtOAc) was concentrated, and purified by FC (heptane/EtOAc 7:3) to give 9H-fluoren-9-ylmethyl 4-[[1-methyl-4-(1-tetrahydropyran-2-ylindazol-5-yl)pyrrolo[2,3-b]pyridin-2-yl]methyl]-piperazine-1-carboxylate (0.18 g). This material was combined with another batch prepared on 8 g scale and stirred 2 h in DMF/piperidine (1.3:1, 40 mL) at 0° C. to RT, concentrated, and purified by FC (DCM/MeOH 9:1) to give Int. 1N1 5-(1-Methyl-2-(piperazin-1-ylmethyl)-1H-pyrrolo[2,3-b]pyridin-4-yl)-1-(tetrahydro-2H-pyran-2-yl)-1H-indazole (2.5 g).

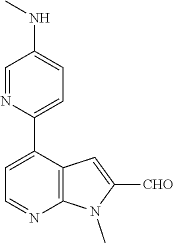 [SMILES: CNc1ccc(-c2ccnc3c2cc(C=O)n3C)nc1]

4,4,5,5-Tetramethyl-2-(4,4,5,5-tetramethyl-1,3,2-dioxaborolan-2-yl)-1,3,2-dioxaborolane (0.27 g), Int. 1AB8 (0.1 g), KOAc (82 mg), and PdDPPFCl₂-DCM (15 mg) were degassed in dioxane (2.5 mL) and stirred at 100° C. ON. The mixture was filtered and HPLC-purified to give a mixture of 1-methyl-4-(4,4,5,5-tetramethyl-1,3,2-dioxaborolan-2-yl)pyrrolo[2,3-b]pyridine-2-carbaldehyde and (2-formyl-1-methyl-pyrrolo[2,3-b]pyridin-4-yl)boronic acid (43 mg). 0.35 g of this mixture, 6-bromo-N-methylpyridin-3-amine (0.39 g), PdDPPFCl₂-DCM (0.14 g), and K₂CO₃ (0.71 g) were degassed in DMF/water (5:1, 0.7 mL) and stirred 1 h at 100° C. The OL (EtOAc/water) was washed with brine, dried, concentrated, and purified by FC (heptane/EtOAc 1:0 to 7:3) to give Int. 1N6/104 1-methyl-4-[5-(methylamino)-2-pyridyl]pyrrolo[2,3-b]pyridine-2-carbaldehyde (0.21 g).

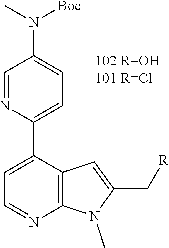

Int. 103 (0.38 g), tert-butyl (6-bromopyridin-3-yl)(methyl)carbamate (0.35 g), PdDPPFCl₂-DCM (0.10 g), and K₂CO₃ (0.50 g) were degassed in dioxane/water (5:1, 4.8 mL) and stirred at 110° C. ON. The OL (water/EtOAc) was washed with brine, concentrated, purified by FC (EtOAc) to give Int. 102 tert-butyl N-[6-[2-(hydroxymethyl)-1-methyl-pyrrolo[2,3-b]-pyridin-4-yl]-3-pyridyl]-N-methyl-carbamate (0.15 g). Int. 102 (90 mg) was stirred 2 h in DCM/Et₃N (80:1, 8 mL) and MsCl (28 μL) at 0° C. to RT. The OL (water/EtOAc) was washed with sat. aq. NaHCO₃, dried, and concentrated to give Int. 101 tert-butyl (6-(2-(chloromethyl)-1-methyl-1H-pyrrolo[2,3-b]pyridin-4-yl)pyridin-3-yl)(methyl)-carbamate (90 mg).

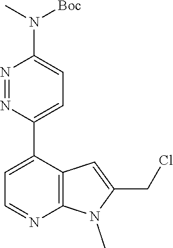 [SMILES: CN(C(=O)OC(C)(C)C)c1ccc(-c2ccnc3c2cc(CCl)n3C)nn1]

Int. 103 (0.54 g), PdDPPFCl₂-DCM (0.14 g), and tert-butyl N-(6-bromopyridazin-3-yl)-N-methyl-carbamate (0.5 g) were degassed in dioxane/water (4.3:1, 8 mL) and stirred at 110° C. ON. The mixture was filtered, concentrated, and purified by FC (hexane/EtOAc 3:7) to give tert-butyl N-[6-[2-(hydroxymethyl)-1-methyl-pyrrolo[2,3-b]pyridin-4-yl]pyridazin-3-yl]-N-methyl-carbamate (0.5 g). 0.2 g of this material was stirred in DCM/Et₃N (11.4:1, 5.5 mL) and MsCl (0.13 mL) at 0° C. to RT and concentrated to give Int. 1P1 tert-butyl N-[6-[2-(chloromethyl)-1-methyl-pyrrolo[2,3-b]pyridin-4-yl]pyridazin-3-yl]-N-methyl-carbamate (0.18 g).

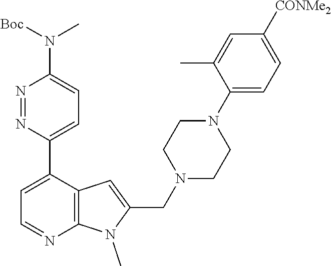

Ints. 1S7/1P1 (0.26 g/0.20 g, TFA salt) and KI (9 mg) were stirred ON in DMF/DIPEA (6.9:1, 2.3 mL) at 0° C. to RT. The OL (EtOAc/water) was dried, concentrated, and purified by FC (DCM/MeOH 9:1) to give Int. 1P2 tert-butyl (6-(2-((4-(4-(dimethylcarbamoyl)-2-methylphenyl)piperazin-1-yl)methyl)-1-methyl-1H-pyrrolo[2,3-b]pyridin-4-yl)pyridazin-3-yl)(methyl)carbamate (0.2 g)

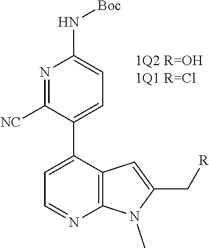

Int. 103 (0.69 g), tert-butyl (5-bromo-6-cyanopyridin-2-yl)carbamate (0.50 g), PdDPPFCl₂-DCM (0.14 g), and K₂CO₃ (0.69 g) were degassed in dioxane/water (5:1, 12 mL) and stirred ON at 110° C. The OL (EtOAc/water) was washed with brine, dried, concentrated, and purified by FC (EtOAc) to give Int. 1Q2 tert-butyl N-[6-cyano-5-[2-(hydroxymethyl)-1-methyl-pyrrolo[2,3-b]pyridin-4-yl]-2-pyridyl]carbamate (0.40 g). Int. 1Q2 (0.10 g) was stirred 2 h in DCM/Et₃N (53:1, 8.2 mL) and MsCl (0.03 mL). The OL (EtOAc/water) was washed with sat. aq. NaHCO₃ and water, dried, and concentrated to give Int. 1Q1 tert-butyl N-[5-[2-(chloromethyl)-1-methyl-pyrrolo[2,3-b]pyridin-4-yl]-6-cyano-2-pyridyl]carbamate (0.10 g).

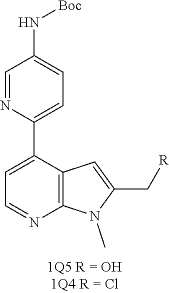

Int. 103 (0.40 g), tert-butyl N-(6-bromo-3-pyridyl)carbamate (0.80 g), PdDPPFCl₂-DCM (0.16 g), and K₂CO₃ (0.8 g) were degassed in dioxane/water (5:1, 6 mL) and stirred ON at 110° C. The OL (EtOAc/water) was washed with brine, dried, concentrated, and purified by FC (EtOAc) to give Int. 1Q5 tert-butyl N-[6-[2-(hydroxymethyl)-1-methyl-pyrrolo[2,3-b]pyridin-4-yl]-3-pyridyl]-carbamate (0.35 g). Int. 1Q5 (0.10 g) was stirred 2 h in DCM/Et₃N (50:1, 5.1 mL) and MsCl (34 μL). The OL (EtOAc/water) was washed with sat. aq. NaHCO₃, dried, and concentrated to give Int. 1Q4 tert-butyl N-[6-[2-(chloromethyl)-1-methyl-pyrrolo[2,3-b]pyridin-4-yl]-3-pyridyl]carbamate (0.10 g).

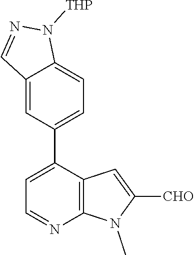

PdDPPFCl₂-DCM (1.37 g), 4-bromo-1-methyl-1H-pyrrolo[2,3-b]pyridine-2-carbaldehyde (8 g), and 1-(tetra-hydro-2H-pyran-2-yl)-5-(4,4,5,5-tetramethyl-1,3,2-dioxaborolan-2-yl)-1H-indazole (13.2 g) were stirred ON in 2M aq. NaHCO₃/dioxane/water (2.3:5:3:1, 130 mL) at 110° C. ON, filtered, and diluted with EtOAc. The OL was dried, concentrated, and purified by FC (hexane/EtOAc 1:3) to give Int. 1R7 1-methyl-4-(1-(tetrahydro-2H-pyran-2-yl)-1H-indazol-5-yl)-1H-pyrrolo[2,3-b]pyridine-2-carbaldehyde (3.8).

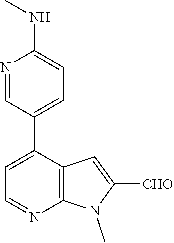 [SMILES: CNc1ccc(-c2ccnc3c2cc(C=O)n3C)cn1]

Int. 1AB8 (0.40 g), NaHCO₃ (0.28 g), N-methyl-5-(4,4,5,5-tetramethyl-1,3,2-dioxaborolan-2-yl)pyridin-2-amine (0.47 g), and PdDPPFCl₂-DCM (0.14 g) were degassed in dioxane/water (9:1, 10 mL) and stirred 2 h at 110° C. under MW conditions, concentrated, and purified by FC (hexane/EtOAc 1:1) to give Int. 1S1 1-methyl-4-[6-(methylamino)-3-pyridyl]pyrrolo[2,3-b]pyridine-2-carbaldehyde (0.32 g).

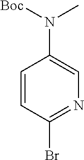 [SMILES: CN(C(=O)OC(C)(C)C)c1ccc(Br)nc1]

tert-Butyl N-(6-bromo-3-pyridyl)carbamate (5.0 g), Cs₂CO₃ (18.0 g) were stirred 2 h in DMF/CH₃I (6.2:1, 17.3 mL) at 0° C. to 50° C. The OL (EtOAc/water) was washed with water, brine, dried, concentrated, and purified by FC (pentane/EtOAc 95:5) to give Int. 1Y4 tert-butyl (6-bromopyridin-3-yl)(methyl)carbamate (4.8 g).

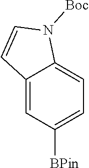

tert-Butyl 5-bromo-1H-indole-1-carboxylate (7.0 g), B₂Pin₂ (7.2 g), PdDPPFCl₂-DCM (0.97 g), and KOAc (7.0 g) were degassed in dioxane/water (11.7:1, 76 mL) and stirred at 100° C. ON. The mixture was filtered, concentrated, and purified by FC (hexane/EtOAc 1:0 to 95:5) to give Int. 1Z5 tert-butyl 5-(4,4,5,5-tetramethyl-1,3,2-dioxaborolan-2-yl)-1H-indole-1-carboxylate (3.7 g).

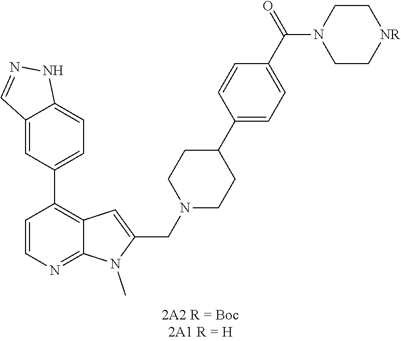

Int. 3A2 (0.20 g, HCl salt), tert-butyl piperazine-1-carboxylate (64 mg), and HATU (0.16 g) were stirred 2 h in DMF/DIPEA (5.7:1, 4 mL). The OL (EtOAc/water) was washed with brine, dried, concentrated, and HPLC-purified to give Int. 2A2 tert-butyl 4-[4-[1-[[1-methyl-4-(1-tetrahydro-pyran-2-ylindazol-5-yl)pyrrolo[2,3-b]-pyridin-2-yl]methyl]-4-piperidyl]benzoyl]-piperazine-1-carboxylate (0.21 g). This material was stirred 1 h in DCM/TFA (2:1, 6 mL). The mixture purified by SCX to give Int. 2A1 (4-(1-((4-(1H-indazol-5-yl)-1-methyl-1H-pyrrolo[2,3-b]pyridin-2-yl)methyl)piperidin-4-yl)phenyl)(piperazin-1-yl)methanone (0.15 g).

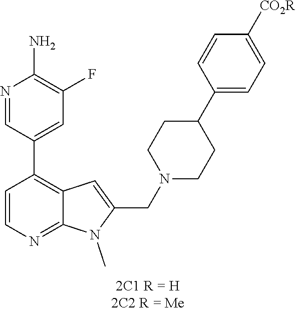

Int. 1AC5 (0.20 g), 3-fluoro-5-(4,4,5,5-tetramethyl-1,3,2-dioxaborolan-2-yl)pyridin-2-amine (0.34 g), PdDPPFCl₂-DCM (55 mg), and NaHCO₃ (0.19 g) were deassed in dioxane/water (10:1, 2.7 mL) and stirred ON at 90° C. The residue after filtration and concentration was purified by FC (heptane/EtOAc/MeOH 1:0:0 to 0:95:5) to give Int. 2C2 methyl 4-[1-[[4-(6-amino-5-fluoro-3-pyridyl)-1-methyl-pyrrolo[2,3-b]pyridin-2-yl]methyl]-4-piperidyl]-benzoate (0.19 g). This material was stirred 1 h in THF/MeOH/2M aq. NaOH (4:2:1, 7 mL). 2M aq. NaOH (1 mL) was added and stirring continued ON and concentrated. The AL (EtOAc/water) was acidified with aq. HCl to precipitate Int. 2C1 4-(1-((4-(6-amino-5-fluoropyridin-3-yl)-1-methyl-1H-pyrrolo[2,3-b]pyridin-2-yl)methyl)-piperidin-4-yl)benzoic acid (0.15 g) after partial concentration.

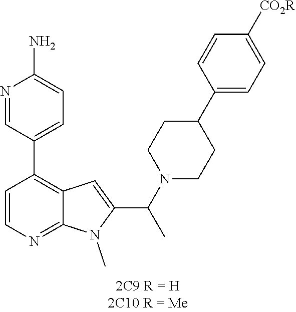

Int. 3E37 (0.15 g), 5-(4,4,5,5-tetramethyl-1,3,2-dioxaborolan-2-yl)pyridin-2-amine (0.11 g), PdDPPFCl₂-DCM (27 mg), and Na₂CO₃ (0.14 g) were degassed in dioxane/water (7:1, 3.6 mL and stirred overnight at 90° C. The OL (EtOAc/brine) was dried, concentrated, and purified by FC (heptane to EtOAc to EtOAc:MeOH (3:2)) to give Int. 2C10 methyl 4-[1-[1-[4-(6-amino-3-pyridyl)-1-methyl-pyrrolo[2,3-b]pyridin-2-yl]ethyl]-4-piperidyl]benzoate (153 mg). This material was stirred ON in THF/MeOH/2M aq. NaOH (1.8:0.9:1, 3.7 mL), neutralized, and HPLC-purified to give Int. 2C9 4-(1-(1-(4-(6-aminopyridin-3-yl)-1-methyl-1H-pyrrolo[2,3-b]pyridin-2-yl)ethyl)piperidin-4-yl)benzoic acid (95 mg).

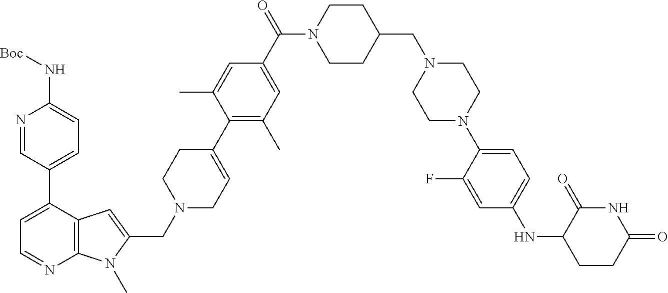 [SMILES: Cc1cc(C(=O)N2CCC(CN3CCN(c4ccc(NC5CCC(=O)NC5=O)cc4F)CC3)CC2)cc(C)c1C1=CCN(Cc2cc3c(-c4ccc(NC(=O)OC(C)(C)C)nc4)ccnc3n2C)CC1]

Ints. 3E41/2C15 (20/16 mg) and HATU (16 mg) were stirred 2 h in DCM/DIPEA/DMF (6:1:1.2, 0.41 mL). The OL (sat. aq. NaHCO₃/DCM) was concentrated and purified by FC (DCM/MeOH 1:0 to 96:4) to give Int. 2C18 tert-butyl (5-(2-((4-(4-(4-((4-(4-((2,6-dioxopiperidin-3-yl)amino)-2-fluorophenyl)piperazin-1-yl)methyl)piperidine-1-carbonyl)-2,6-dimethylphenyl)-3,6-dihydropyridin-1(2H)-yl)methyl)-1-methyl-1H-pyrrolo[2,3-b]pyridin-4-yl)pyridin-2-yl)carbamate (22 mg).

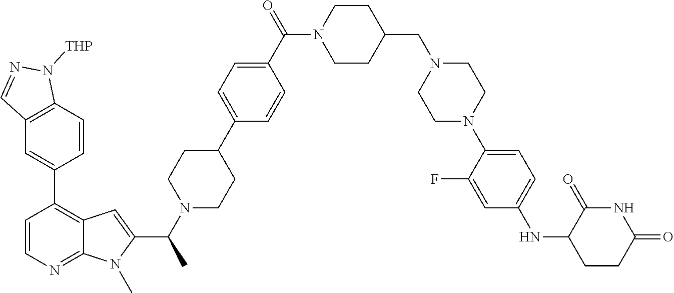

Int. 2C19 3-((3-fluoro-4-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.19 g) prepared similarly to Int. 2C18 from Ints. 2C54/2C15 (0.12 g/0.12 g, TFA salt).

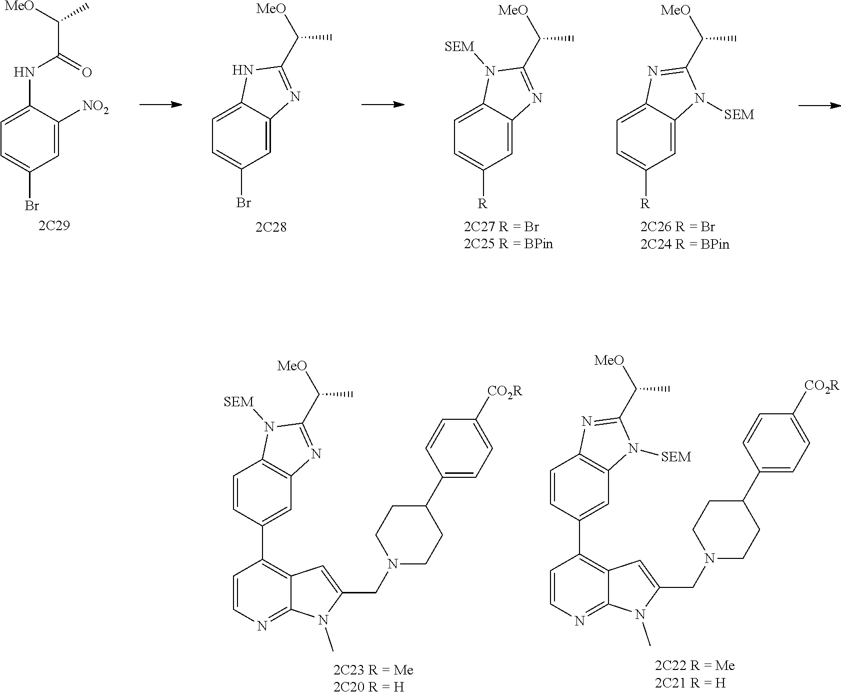

(R)-2-Methoxypropanoic acid (2.1 g) was stirred 1 h in pyridine/POCl₃ (15.4:1, 42.6 mL) at 0° C. 4-Bromo-2-nitroaniline (4.0 g) was added and stirring continued at 0° C. to RT ON. The mixture was diluted with aq. HCl to precipitate Int. 2C29 (R)—N-(4-bromo-2-nitro-phenyl)-2-methoxy-propanamide (4.0 g g). This material and iron powder (2.2 g) were stirred 4 h at 65° C. in EtOH:AcOH (1:1.75, 55 mL) and filtered. The OL (EtOAc/water) was dried, concentrated, and was washed with Et₂O and dried to give Int. 2C28 5-bromo-2-[(R)-1-methoxyethyl]-1H-benzimidazole (2.5 g). This material and NaH (60% oil dispersion; 0.35 g) were stirred 0.5 h in DMF (5 mL) at 0° C. SEM-Cl (2.0 g) was added and stirring continued 3 h at 0° C. to RT. The OL (EtOAc/water) was dried, concentrated, and purified by FC (hexane/EtOAc 4:1) to give Ints. 2C26/2C27 (R)-6-bromo-2-(1-methoxyethyl)-1-((2-(trimethylsilyl)-ethoxy)methyl)-1H-benzo[d]-imidazole and (R)-5-bromo-2-(1-methoxy-ethyl)-1-((2-(trimethyl-silyl)ethoxy)-methyl)-1H-benzo[d]-imidazole (2.0 g). 1.5 g of this mixture, PdDPPFCl₂-DCM (0.32 g), B₂Pin₂ (2.0 g), and KOAc (1.1 g) were degassed in dioxane (5 mL) and stirred ON at 100° C. The OL (EtOAc/water) was dried, concentrated, and purified by FC (hexane/EtOAc 55:45) to give Ints. 2C25/2C24 (R)-2-(1-methoxyethyl)-5-(4,4,5,5-tetramethyl-1,3,2-dioxa-borolan-2-yl)-1-((2-(trimethylsilyl)-ethoxy)methyl)-1H-benzo[d]imidazole and (R)-2-(1-methoxy-ethyl)-6-(4,4,5,5-tetramethyl-1,3,2-dioxaborolan-2-yl)-1-((2-(trimethylsilyl)ethoxy)-methyl)-1H-benzo[d]-imidazole (1.4 g). 0.24 g of this mixture, Int. 1AC5 (0.25 g), PdDPPFCl₂-DCM (45 mg), and NaHCO₃ (92 mg) were degassed in dioxane/water (6:1, 3.5 mL) and stirred ON at 100° C. The OL (EtOAc/water) was dried, concentrated, and purified by FC (pentane/EtOAc 4:1 to 7:3) to give Ints. 2C22/2C23 methyl (R)-4-(1-((4-(2-(1-methoxy-ethyl)-1-((2-(trimethylsilyl)ethoxy)methyl)-1H-benzo[d]-imidazol-5-yl)-1-methyl-1H-pyrrolo[2,3-b]pyridin-2-yl)methyl)piperidin-4-yl)benzoate and methyl (R)-4-(1-((4-(2-(1-methoxyethyl)-1-((2-(trimethylsilyl)-ethoxy)-methyl)-1H-benzo[d]imidazol-6-yl)-1-methyl-1H-pyrrolo[2,3-b]pyridin-2-yl)methyl)-piperidin-4-yl)-benzoate (0.2 g). This mixture and LiOH—H₂O (37 mg) were stirred ON in THF/MeOH/water (2:2:1, 2.5 mL) and concentrated. The residue was triturated in aq. citric acid to give Ints. 2C20/2C21 (R)-4-(1-((4-(2-(1-methoxyethyl)-1-((2-(trimethylsilyl)ethoxy)methyl)-1H-benzo[d]-imidazol-5-yl)-1-methyl-1H-pyrrolo[2,3-b]pyridin-2-yl)methyl)piperidin-4-yl)benzoic acid and (R)-4-(1-((4-(2-(1-methoxy-ethyl)-1-((2-(trimethylsilyl)ethoxy)methyl)-1H-benzo[d]imidazol-6-yl)-1-methyl-1H-pyrrolo[2,3-b]pyridin-2-yl)-methyl)piperidin-4-yl)benzoic acid (0.19 g).

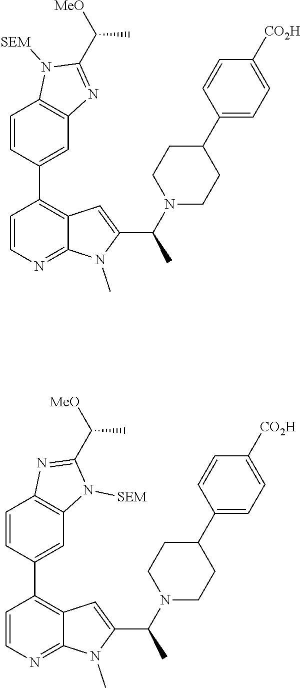 [SMILES: CO[C@H](C)c1nc2cc(-c3ccnc4c3cc([C@H](C)N3CCC(c5ccc(C(=O)O)cc5)CC3)n4C)ccc2n1COCC[Si](C)(C)C ; CO[C@H](C)c1nc2ccc(-c3ccnc4c3cc([C@H](C)N3CCC(c5ccc(C(=O)O)cc5)CC3)n4C)cc2n1COCC[Si](C)(C)C]

Int. 2C34/2C35 4-(1-((S)-1-(4-(2-((R)-1-methoxyethyl)-1-((2-(trimethylsilyl)ethoxy)methyl)-1H-benzo[d]imidazol-5-yl)-1-methyl-1H-pyrrolo[2,3-b]pyridin-2-yl)ethyl)piperidin-4-yl)benzoic acid and 4-(1-((S)-1-(4-(2-((R)-1-methoxyethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoic acid were prepared similarly to Ints. 2C20/2C21 from (S)-2-methoxypropanoic acid.

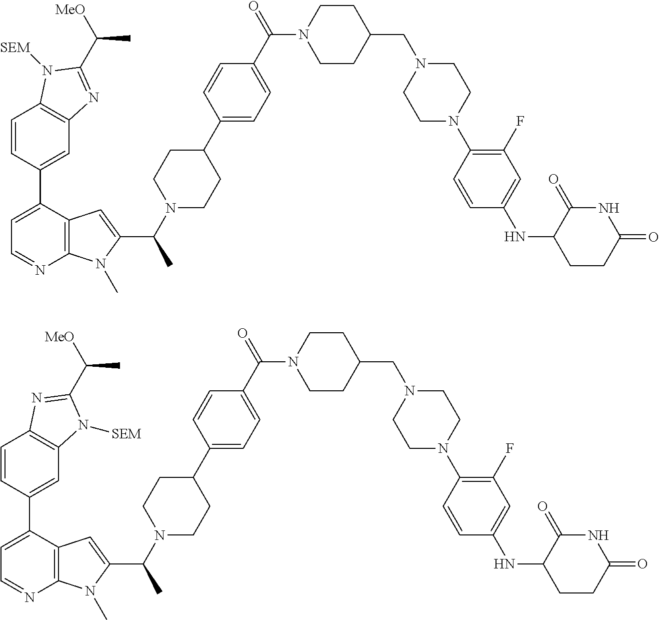 [SMILES: CO[C@@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C30 and 2C31. 3-((3-fluoro-4-(4-((1-(4-(1-((S)-1-(4-(2-((S)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)amino)piperidine-2,6-dione and 3-((3-fluoro-4-(4-((1-(4-(1-((S)-1-(4-(2-((S)-1-methoxyethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.30 g) were prepared similarly to Int. 2C18 from Ints. 2C20/2C21/2C15 (0.20 g/0.29 g, HCl salt).

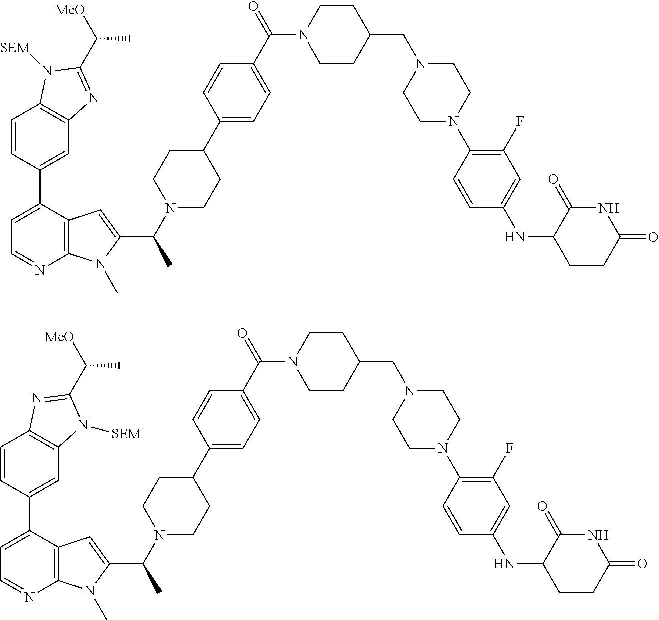 [SMILES: CO[C@H](C)c1nc2cc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)ccc2n1COCC[Si](C)(C)C ; CO[C@H](C)c1nc2ccc(-c3ccnc4c3cc([C@H](C)N3CCC(c5ccc(C(=O)N6CCC(CN7CCN(c8ccc(NC9CCC(=O)NC9=O)cc8F)CC7)CC6)cc5)CC3)n4C)cc2n1COCC[Si](C)(C)C]

Int. 2C32 and 2C33. 3-((3-fluoro-4-(4-((1-(4-(1-((S)-1-(4-(2-((R)-1-methoxyethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)phenyl)amino)piperidine-2,6-dione and 3-((3-fluoro-4-(4-((1-(4-(1-((S)-1-(4-(2-((R)-1-methoxyethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)-piperidine-2,6-dione (0.15 g) were prepared similarly to Int. 2C18 from Int. 2C34/2C35/2C15 (0.19 g/0.22 g, HCl salt).

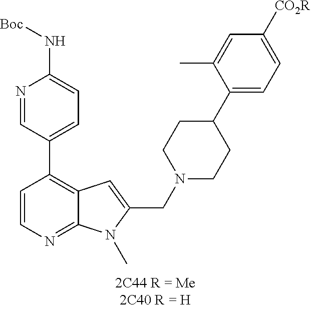

Methyl 4-bromo-3-methylbenzoate (2.5 g), tert-butyl 4-(4,4,5,5-tetramethyl-1,3,2-dioxaborolan-2-yl)-3,6-dihydropyridine-1(2H)-carboxylate (4.0 g), NaHCO₃ (2.8 g), and PdDPPFCl₂-DCM (0.45 g) were degassed in dioxane/water (10:1, 30 mL) and stirred ON at 100° C. The mixture was diluted with EtOAc (100 mL), filtered, concentrated, and purified by FC (hexane/EtOAc 9:1 to 85:15) to give Int. 2C41 tert-butyl 4-(4-methoxy-carbonyl-2-methyl-phenyl)-3,6-dihydro-2H-pyridine-1-carboxylate (3.2 g). Int. 2C41 (0.25 g) was stirred ON in DCM/4M HCl in dioxane (4:1, 10 mL) and concentrated. The residue was triturated in pentane to give Int. 2C42 methyl 3-methyl-4-(1,2,3,6-tetrahydropyridin-4-yl)benzoate (0.19 g, HCl salt). Ints. 1AB8/2C42 (1.2 g/1.2 g, HCl salt) were stirred 4 h in DCE/DIPEA (2:2:1, 7.3 mL) at 0° C. to RT. STAB (1.9 g) was added and stirring continued ON. The OL (EtOAc/water) was dried, concentrated, and purified by FC (hexane/EtOAc 7:3) to give Int. 2C43 methyl 4-(1-((4-bromo-1-methyl-1H-pyrrolo[2,3-b]pyridin-2-yl)methyl)-1,2,3,6-tetrahydropyridin-4-yl)-3-methylbenzoate (1.0 g). This material, tert-butyl (5-(4,4,5,5-tetramethyl-1,3,2-dioxaborolan-2-yl)pyridin-2-yl)carbamate (0.78 g), NaHCO₃ (0.28 g), and PdDPPFCl₂-DCM (90 mg) were degassed in dioxane/water (9:1, 8 mL) and stirred ON at 90° C. The OL (EtOAc/water) was concentrated and purified by FC (pentane/EtOAc 7:3) to give Int. 2C44 methyl 4-[1-[[4-[6-(tert-butoxycarbonylamino)-3-pyridyl]-1-methyl-pyrrolo[2,3-b]pyridin-2-yl]-methyl]-3,6-dihydro-2H-pyridin-4-yl]-3-methyl-benzoate (0.46 g). This material and LiOH—H₂O (0.17 g) were stirred ON in THF/MeOH/water (6:2:1, 7 mL). The mixture was neutralized with 0.5M aq. HCl to precipitate Int. 2C40 4-(1-((4-(6-((tert-butoxycarbonyl)amino)pyridin-3-yl)-1-methyl-1H-pyrrolo[2,3-b]pyridin-2-yl)methyl)piperidin-4-yl)-3-methylbenzoic acid (0.18 g).

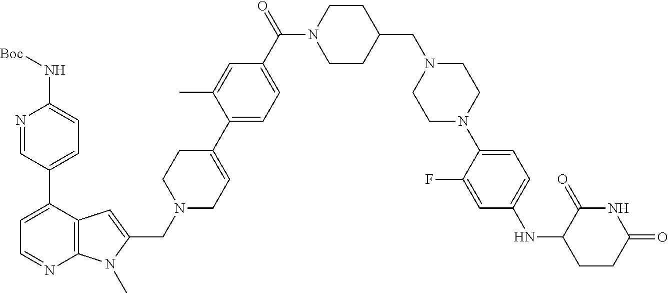 [SMILES: Cc1cc(C(=O)N2CCC(CN3CCN(c4ccc(NC5CCC(=O)NC5=O)cc4F)CC3)CC2)ccc1C1=CCN(Cc2cc3c(-c4ccc(NC(=O)OC(C)(C)C)nc4)ccnc3n2C)CC1]

Int. 2C45. tert-butyl (5-(2-((4-(4-(4-((4-(4-((2,6-dioxopiperidin-3-yl)amino)-2-fluorophenyl)-piperazin-1-yl)methyl)piperidine-1-carbonyl)-2-methylphenyl)-3,6-dihydropyridin-1(2H)-yl)methyl)-1-methyl-1H-pyrrolo[2,3-b]pyridin-4-yl)pyridin-2-yl)carbamate (27 mg) was prepared similarly to Int. 2C18 from Ints. 2C40/2C15 (25 mg/20 mg, HCl salt).

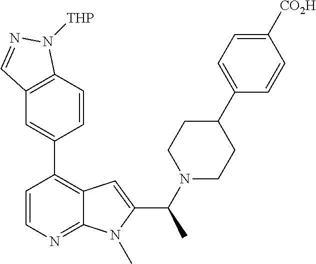

Int. 3E39 (59 mg) was stirred ON in MeOH/THF/1M aq. NaOH (2.6:1:3:1, 2 mL) at 0° C. to RT. The mixture was partially concentrated. The OL (brine and 1M aq. HCl/DCM/MeOH (9:1)) was dried, concentrated, and purified by FC (DCM/MeOH 95:5) to give Int. 2C54 4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoic acid (47 mg).

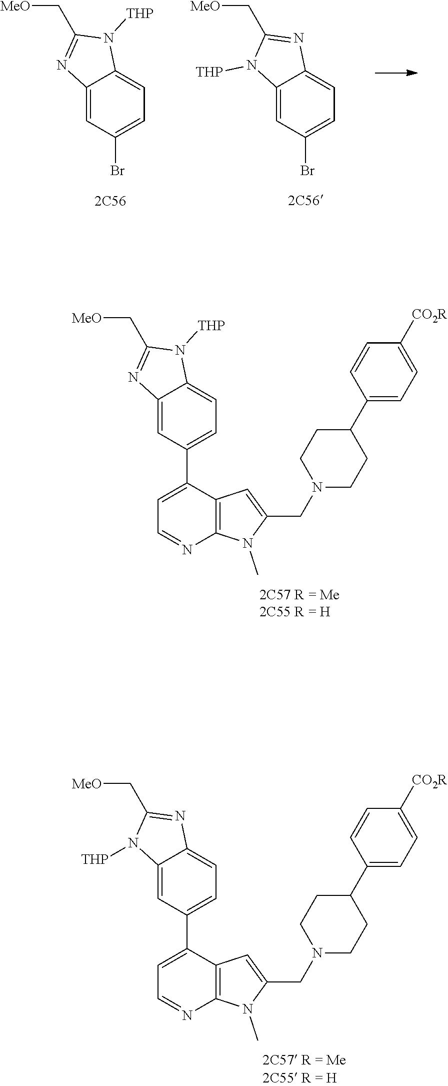

5-Bromo-2-(methoxymethyl)-1H-benzo[d]imidazole (1.0 g) and TsOH-H₂O (0.16 g) were stirred ON in THF/3,4-dihydropyran (4:1, 12.3 mL) at 80° C. The OL (EtOAc/water) was washed with brine, dried, concentrated, and purified by FC (pentane/EtOAc 5:1 to 4:1) to give Ints. 2C56/2C56′ 5-bromo-2-(methoxymethyl)-1-(tetrahydro-2H-pyran-2-yl)-1H-benzo[d]imidazole/6-bromo-2-(methoxymethyl)-1-(tetrahydro-2H-pyran-2-yl)-1H-benzo[d]imidazole (0.50 g). Ints. 2C56/2C56′/3E170 (0.40 g/0.72 g), NaHCO₃ (0.31 g), and Pd(PPh₃)₄ (50 mg) were degassed in dioxane/water (4:1, 5 mL) and stirred ON at 90° C. Filtration, concentration, and purification by FC (hexane/EtOAc 1:1) gave Ints. 2C57/2C57′ methyl 4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoate/methyl 4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoate (70 mg). Int. 2C57 (1.0 g) and LiOH—H₂O (0.35 g) were stirred ON in THF/MeOH/water (3:3:1, 5 mL) at 0° C. to RT and concentrated. The residue was triturated with dilute aq. HCl to give Ints. 2C55/2C55′ 4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoic acid/4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoic acid (0.58 g, HCl salt).

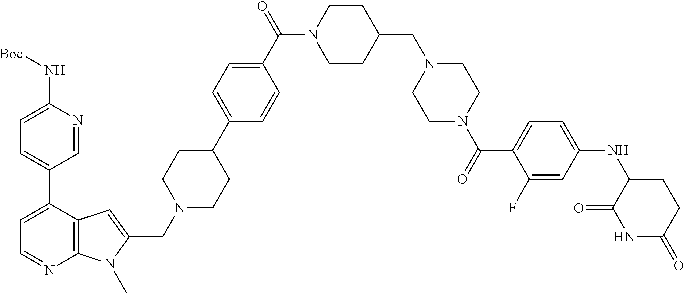 [SMILES: Cn1c(CN2CCC(c3ccc(C(=O)N4CCC(CN5CCN(C(=O)c6ccc(NC7CCC(=O)NC7=O)cc6F)CC5)CC4)cc3)CC2)cc2c(-c3ccc(NC(=O)OC(C)(C)C)nc3)ccnc21]

Int. 2E1. tert-butyl (5-(2-((4-(4-(4-((4-(4-((2,6-dioxopiperidin-3-yl)amino)-2-fluorobenzoyl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (33 mg) was prepared similarly to Int. 2C18 from Ints. 2E6/2E2 (25 mg/32 mg, TFA salt).

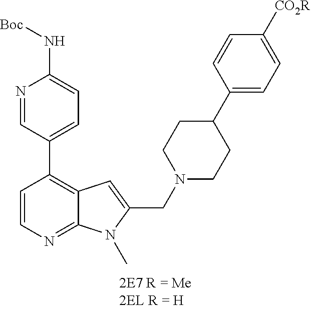

PdDPPFCl₂-DCM (0.37 g), Int. 1AC5 (2.0 g), tert-butyl (5-(4,4,5,5-tetramethyl-1,3,2-dioxaborolan-2-yl)pyridin-2-yl)carbamate (1.6 g), and NaHCO₃ (0.76 g), were degassed in dioxane/water (10:2, 22 mL) and stirred ON at 80° C. The OL (water/EtOAc) was dried, concentrated, and purified by FC (hexane/EtOAc 3:7) to give Int. 2E7 methyl 4-(1-((4-(6-((tert-butoxycarbonyl)amino)pyridin-3-yl)-1-methyl-1H-pyrrolo[2,3-b]pyridin-2-yl)-methyl)-piperidin-4-yl)benzoate (2.1 g). This material and LiOH—H₂O (0.93 g) were stirred 5 h in water/MeOH/THF (1:2:2, 10 mL) at 0° C. and concentrated. The residue was triturated in dilute aq. HCl to give Int. 2E6 4-(1-((4-(6-((tert-butoxycarbonyl)amino)-pyridin-3-yl)-1-methyl-1H-pyrrolo[2,3-b]pyridin-2-yl)methyl)piperidin-4-yl)benzoic acid (1.7 g).

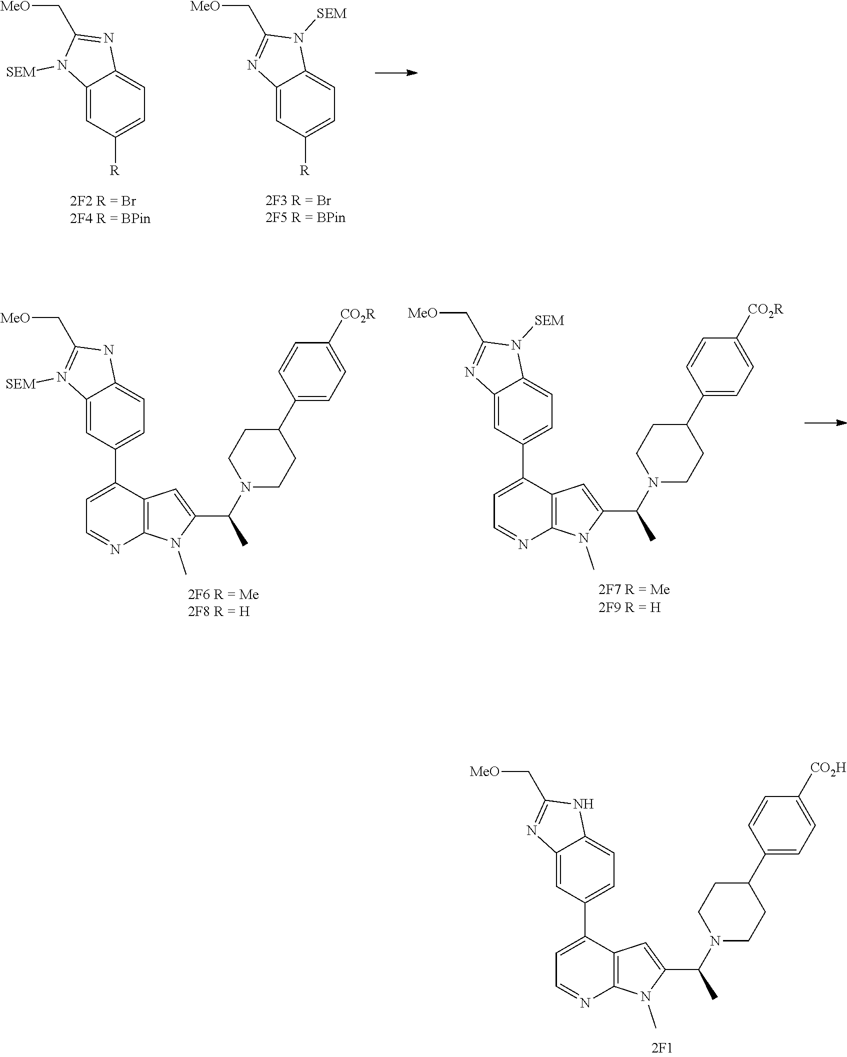

5-Bromo-2-(methoxymethyl)-1H-benzo[d]imidazole (25 g) was added to a mixture of NaH (60% oil dispersion, 6.22 g) in DMF (150 mL) at 0° C. SEM-Cl (20.8 g) was added after 0.25 h at 0° C. The OL (water/EtOAc) was dried and concentrated. This material was mixed with another batch prepared similarly on 10 g scale and purified by FC (pentane/EtOAc 3:7) to give Ints. 2F2/2F3 6-bromo-2-(methoxy-methyl)-1-((2-(trimethylsilyl)ethoxy)methyl)-1H-benzo[d]-imidazole and 5-bromo-2-(methoxy-methyl)-1-((2-(trimethylsilyl)-ethoxy)methyl)-1H-benzo[d]-imidazole (30 g). 3.0 g of this mixture, B₂Pin₂ (2.25 g), PdDPPFCl₂-DCM (0.33 g), and KOAc (2.38 g) degassed in dioxane (25 mL) and stirred ON at 90° C. The filtrate after filtration was concentrated and purified by FC (pentane/EtOAc 1:4) to give Ints. 2F4/2F5 2-(methoxy-methyl)-6-(4,4,5,5-tetramethyl-1,3,2-dioxaborolan-2-yl)-1-((2-(trimethylsilyl)ethoxy)methyl)-1H-benzo[d]-imidazole and 2-(methoxymethyl)-5-(4,4,5,5-tetramethyl-1,3,2-dioxaborolan-2-yl)-1-((2-(trimethyl-silyl)ethoxy)-methyl)-1H-benzo[d]imidazole (2.1 g). 0.50 g of this mixture, Int. 3E35 (0.50 g), PdDPPFCl₂-DCM (89 mg), and NaHCO₃ were degassed in dioxane/water (3:1, 20 mL) and stirred ON at 80° C. and filtered. The OL (EtOAc/water) was dried and concentrated. This material was mixed with another batch prepared similarly from Int. 3E35 (2.5 g) and purified by FC (heptane/EtOAc 7:3 to 2:3) to give Ints. 2F6/2F7 methyl (S)-4-(1-(1-(4-(2-(methoxy-methyl)-1-((2-(trimethylsilyl)-ethoxy)-methyl)-1H-benzo[d]-imidazol-6-yl)-1-methyl-1H-pyrrolo[2,3-b]pyridin-2-yl)ethyl)-piperidin-4-yl)-benzoate and methyl (S)-4-(1-(1-(4-(2-(methoxymethyl)-1-((2-(trimethyl-silyl)ethoxy)-methyl)-1H-benzo[d]-imidazol-5-yl)-1-methyl-1H-pyrrolo[2,3-b]pyridin-2-yl)ethyl)piperidin-4-yl)-benzoate (3.2 g). 3.0 g of this mixture and LiOH—H₂O (0.55 g) were stirred ON at 0° C. to RT in THF/MeOH/water (2:2:1, 25 mL). The mixture was partially concentrated and triturated in dilute aq. HCl to give Ints. 2F8/2F9 (S)-4-(1-(1-(4-(2-(methoxy-methyl)-1-((2-(trimethyl-silyl)-ethoxy)methyl)-1H-benzo[d]imidazol-6-yl)-1-methyl-1H-pyrrolo[2,3-b]pyridin-2-yl)ethyl)piperidin-4-yl)benzoic acid and (S)-4-(1-(1-(4-(2-(methoxy-methyl)-1-((2-(trimethylsilyl)-ethoxy)-methyl)-1H-benzo[d]-imidazol-5-yl)-1-methyl-1H-pyrrolo[2,3-b]pyridin-2-yl)ethyl)-piperidin-4-yl)benzoic acid (2.5 g). This material was stirred ON in DCM/4M HCl in dioxane (0.6:1, 40 mL) at 0° C. to RT, concentrated, and triturated in Et₂O to give Int. 2F1 4-[1-[(1S)-1-[4-[2-(methoxymethyl)-1H-benzimidazol-5-yl]-1-methyl-pyrrolo[2,3-b]pyridin-2-yl]ethyl]-4-piperidyl]-benzoic acid (2.0 g, HCl salt).

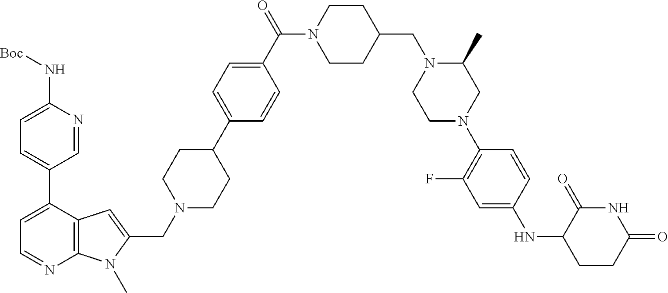 [SMILES: C[C@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F20. tert-butyl (5-(2-((4-(4-(4-(((2S)-4-(4-((2,6-dioxopiperidin-3-yl)amino)-2-fluorophenyl)-2-methylpiperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (50 mg) was prepared similarly to Int. 2C18 from HCl salts of Ints. 2E6/2F21 (48 mg/34 mg).

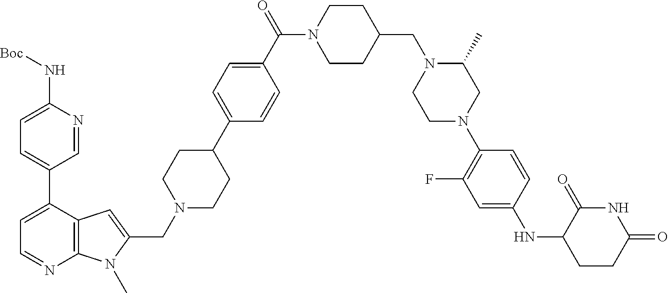 [SMILES: C[C@@H]1CN(c2ccc(NC3CCC(=O)NC3=O)cc2F)CCN1CC1CCN(C(=O)c2ccc(C3CCN(Cc4cc5c(-c6ccc(NC(=O)OC(C)(C)C)nc6)ccnc5n4C)CC3)cc2)CC1]

Int. 2F27. tert-butyl (5-(2-((4-(4-(4-(((2R)-4-(4-((2,6-dioxopiperidin-3-yl)amino)-2-fluorophenyl)-2-methylpiperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (39 mg) was prepared similarly to Int. 2C18 from Ints. 2E6/2F21 (34 mg/HCl salt).

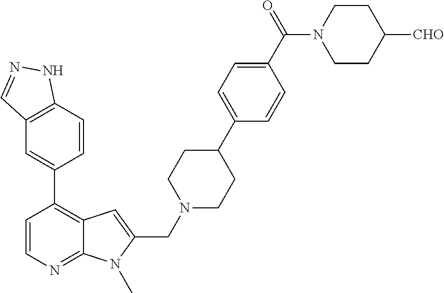 [SMILES: Cn1c(CN2CCC(c3ccc(C(=O)N4CCC(C=O)CC4)cc3)CC2)cc2c(-c3ccc4[nH]ncc4c3)ccnc21]

A solution of Int. 3D4 (0.10 g) in DCM/TFA (2:1, 7.5 mL) was stirred 1 h and concentrated to give Int. 2G1 1-(4-(1-((4-(1H-indazol-5-yl)-1-methyl-1H-pyrrolo[2,3-b]pyridin-2-yl)methyl)piperidin-4-yl)benzoyl)piperidine-4-carbaldehyde (97 mg, TFA salt).

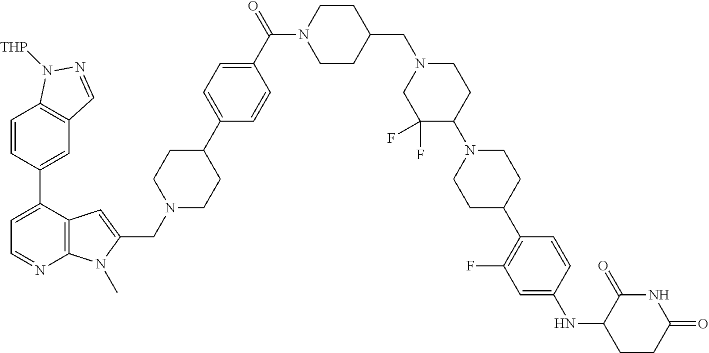

Int. 2H1. 3-((4-(3′,3′-difluoro-1′-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-[1,4′-bipiperidin]-4-yl)-3-fluorophenyl)amino)piperidine-2,6-dione (22 mg) was prepared similarly to Int. 2C18 from HCl salts of Ints. 3A2/2H2 (24 mg/22 mg).

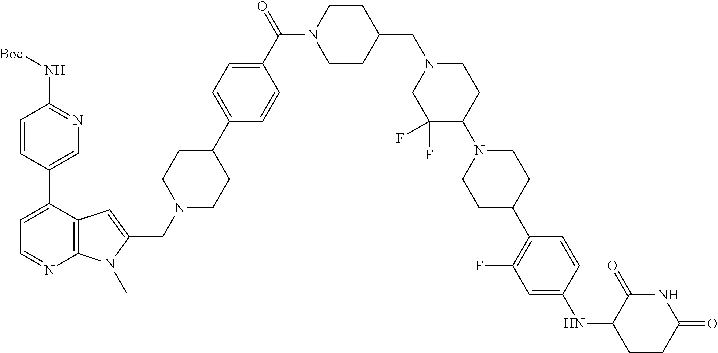 [SMILES: Cn1c(CN2CCC(c3ccc(C(=O)N4CCC(CN5CCC(N6CCC(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2H11. tert-butyl (5-(2-((4-(4-(4-((4-(4-((2,6-dioxopiperidin-3-yl)amino)-2-fluorophenyl)-3′,3′-difluoro-[1,4′-bipiperidin]-1′-yl)methyl)piperidine-1-carbonyl)phenyl)piperidin-1-yl)methyl)-1-methyl-1H-pyrrolo[2,3-b]pyridin-4-yl)pyridin-2-yl)carbamate (33 mg) was prepared similarly to Int. 2C18 from HCl salts of Ints. 2E6/2H2 (20 mg/24 mg).

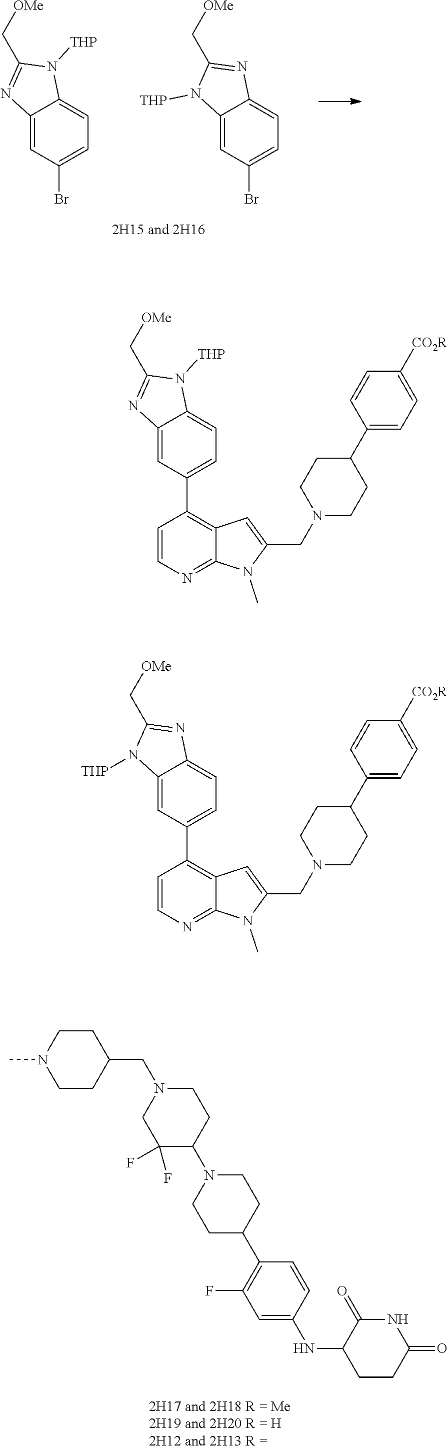

Int. 1AC5 (3.0 g), B₂Pin₂ (1.1 g), KOAc (2.0 g), and PdDPPFCl₂-DCM (50 mg) were degassed in dioxane (30 mL) and stirred ON at 90° C. The filtrate after filtration was concentrated and purified by FC (pentane/EtOAc 1:4) to give Int. 2H14 methyl 4-[i-[[i-methyl-4-(4,4,5,5-tetramethyl-1,3,2-dioxaborolan-2-yl)-pyrrolo[2,3-b]pyridin-2-yl]methyl]-4-piperidyl]benzoate (1.7 g). 5-Bromo-2-(methoxymethyl)-1H-benzo[d]imidazole (10 g), and TsOH-H₂O (1.6 g) were stirred ON at 80° C. in THF/3,4-dihydropyran (4:1, 125 mL). The OL (EtOAc/water) was washed with brine, dried, concentrated, and purified by FC (pentane/EtOAc 4:1 to 3:1) to give Ints. 2H15/2H16 5-bromo-2-(methoxymethyl)-1-(tetrahydro-2H-pyran-2-yl)-1H-benzo[d]imidazole and 6-bromo-2-(methoxy-methyl)-1-(tetrahydro-2H-pyran-2-yl)-1H-benzo[d]-imidazole (5.0 g). Ints. 2H14/2H15/2H16 (4.52 g/2.0 g), NaHCO₃ (1.55 g), and Pd(PPh₃)₄ (0.36 g) were degassed in dioxane/water (5:1, 24 mL) and stirred ON at 90° C., filtered, concentrated, and purified by FC (hexane/EtOAc 1:1) to give Ints. 2H17/2H18 methyl 4-(1-((4-(2-(methoxy-methyl)-1-(tetrahydro-2H-pyran-2-yl)-1H-benzo[d]imidazol-6-yl)-1-methyl-1H-pyrrolo[2,3-b]-pyridin-2-yl)-methyl)piperidin-4-yl)benzoate and methyl 4-(1-((4-(2-(methoxymethyl)-1-(tetra-hydro-2H-pyran-2-yl)-1H-benzo[d]imidazol-5-yl)-1-methyl-1H-pyrrolo[2,3-b]pyridin-2-yl)methyl)-piperidin-4-yl)benzoate (1.1 g). Ints. 2H17/2H18 (1.0 g) and LiOH—H₂O (0.35 g) were stirred 8 h in THF/MeOH/water (3:3:1; 5 mL) at 0° C. to RT, concentrated, and triturated in dilute aq. HCl to give Ints. 2H19/2H20 4-(1-((4-(2-(methoxy-methyl)-1-(tetra-hydro-2H-pyran-2-yl)-1H-benzo[d]imidazol-5-yl)-1-methyl-1H-pyrrolo[2,3-b]pyridin-2-yl)-methyl)-piperidin-4-yl)benzoic acid and 4-(1-((4-(2-(methoxy-methyl)-1-(tetra-hydro-2H-pyran-2-yl)-1H-benzo[d]-imidazol-6-yl)-1-methyl-1H-pyrrolo[2,3-b]pyridin-2-yl)methyl)-piperidin-4-yl)benzoic acid (0.58 g, HCl salt). 18 mg of this mixture and Int. 2H2 (21 mg, HCl salt) were converted to Ints. 2H12/2H13 3-((4-(3′,3′-difluoro-1′-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)-piperidin-4-yl)-methyl)-[1,4′-bipiperidin]-4-yl)-3-fluorophenyl)amino)piperidine-2,6-dione and 3-((4-(3′,3′-difluoro-1′-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]-pyridin-2-yl)methyl)piperidin-4-yl)benzoyl)piperidin-4-yl)methyl)-[1,4′-bi-piperidin]-4-yl)-3-fluorophenyl)-amino)piperidine-2,6-dione (19 mg) similarly to Int. 2C18.

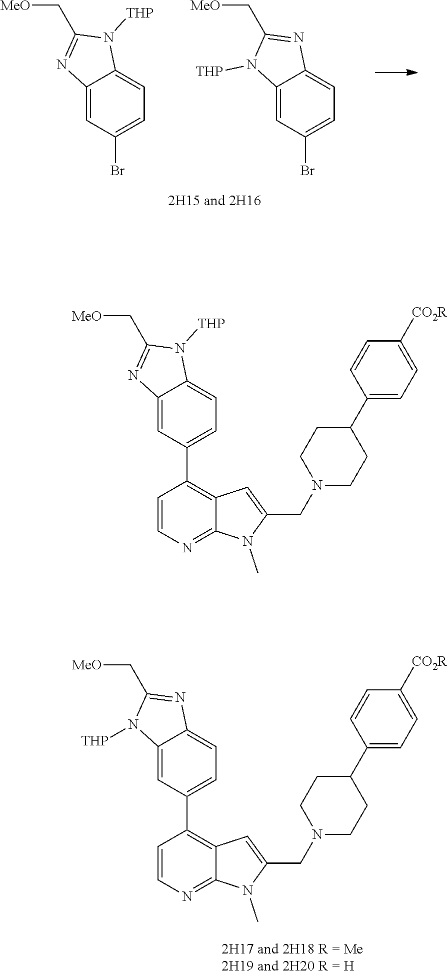

5-Bromo-2-(methoxymethyl)-1H-benzo[d]imidazole (10 g), and TsOH-H₂O (1.6 g) were stirred ON at 80° C. in THF/3,4-dihydropyran (4:1, 125 mL). The OL (EtOAc/water) was washed with brine, dried, concentrated, and purified by FC (pentane/EtOAc 4:1 to 3:1) to give Ints. 2H15/2H16 5-bromo-2-(methoxymethyl)-1-(tetrahydro-2H-pyran-2-yl)-1H-benzo[d]imidazole and 6-bromo-2-(methoxy-methyl)-1-(tetrahydro-2H-pyran-2-yl)-1H-benzo[d]-imidazole (5.0 g). Ints. 2H14/2H15/2H16 (4.52 g/2.0 g), NaHCO₃ (1.55 g), and Pd(PPh₃)₄ (0.36 g) were degassed in dioxane/water (5:1, 24 mL) and stirred ON at 90° C., filtered, concentrated, and purified by FC (hexane/EtOAc 1:1) to give Ints. 2H17/2H18 methyl 4-(1-((4-(2-(methoxy-methyl)-1-(tetrahydro-2H-pyran-2-yl)-1H-benzo[d]imidazol-6-yl)-1-methyl-1H-pyrrolo[2,3-b]-pyridin-2-yl)-methyl)piperidin-4-yl)benzoate and methyl 4-(1-((4-(2-(methoxymethyl)-1-(tetra-hydro-2H-pyran-2-yl)-1H-benzo[d]imidazol-5-yl)-1-methyl-1H-pyrrolo[2,3-b]pyridin-2-yl)methyl)-piperidin-4-yl)benzoate (1.1 g). Ints. 2H17/2H18 (1.0 g) and LiOH—H₂O (0.35 g) were stirred 8 h in THF/MeOH/water (3:3:1; 5 mL) at 0° C. to RT, concentrated, and triturated in dilute aq. HCl to give Ints. 2H19/2H20 4-(1-((4-(2-(methoxy-methyl)-1-(tetra-hydro-2H-pyran-2-yl)-1H-benzo[d]imidazol-5-yl)-1-methyl-1H-pyrrolo[2,3-b]pyridin-2-yl)-methyl)-piperidin-4-yl)benzoic acid and 4-(1-((4-(2-(methoxy-methyl)-1-(tetra-hydro-2H-pyran-2-yl)-1H-benzo[d]imidazol-6-yl)-1-methyl-1H-pyrrolo[2,3-b]pyridin-2-yl)methyl)-piperidin-4-yl)benzoic acid (0.58 g, HCl salt).

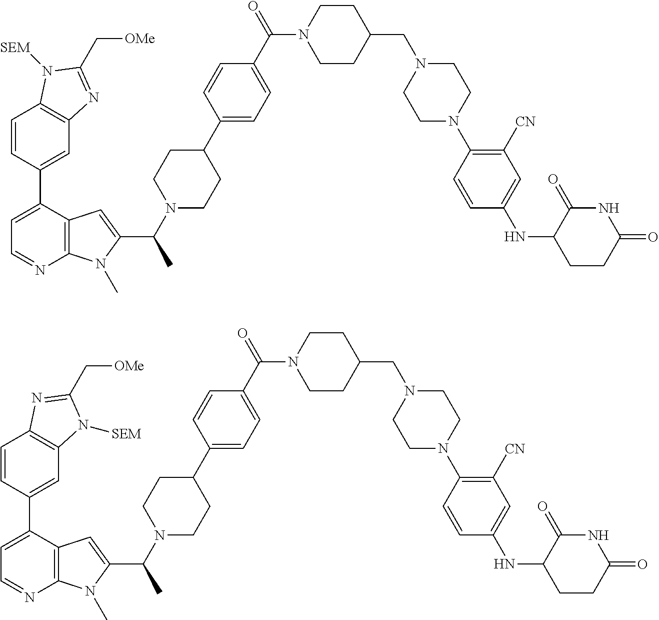 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C#N)CC7)CC6)cc5)CC3)n4C)cc2n1COCC[Si](C)(C)C]

Int. 2I5 and 2I6. 5-((2,6-dioxopiperidin-3-yl)amino)-2-(4-((1-(4-(1-((S)-1-(4-(2-(methoxymethyl)-1-((2-(trimethyl-silyl)ethoxy)methyl)-1H-benzo[d]imidazol-5-yl)-1-methyl-1H-pyrrolo[2,3-b]pyridin-2-yl)ethyl)-piperidin-4-yl)-benzoyl)piperidin-4-yl)methyl)piperazin-1-yl)benzonitrile and 5-((2,6-dioxopiperidin-3-yl)amino)-2-(4-((1-(4-(1-((S)-1-(4-(2-(methoxymethyl)-1-((2-(trimethylsilyl)ethoxy)methyl)-1H-benzo[d]-imidazol-6-yl)-1-methyl-1H-pyrrolo[2,3-b]pyridin-2-yl)ethyl)piperidin-4-yl)benzoyl)piperidin-4-yl)-methyl)piperazin-1-yl)-benzonitrile (0.12 g) was prepared similarly to Int. 2C18 from Ints. 2F8/2F9/2I13 (0.10 g/96 mg, HCl salt).

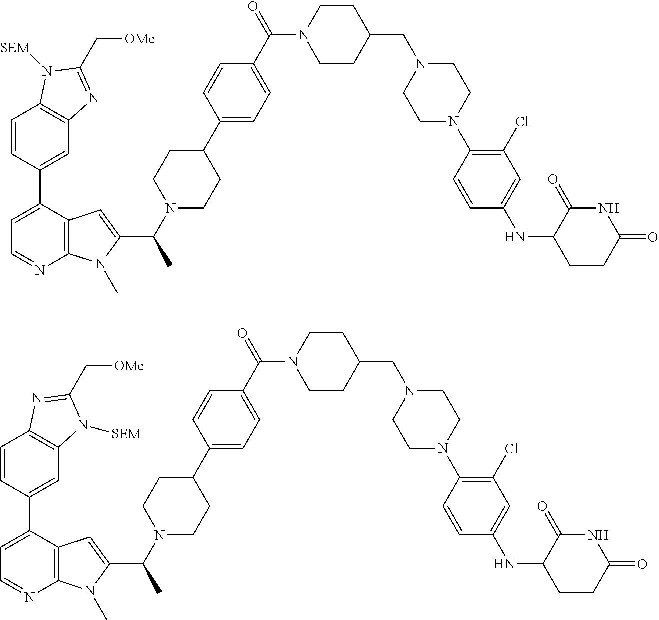 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8Cl)CC7)CC6)cc5)CC3)n4C)cc2n1COCC[Si](C)(C)C]

Int. 217 and 2I8. 3-((3-chloro-4-(4-((1-(4-(1-((S)-1-(4-(2-(methoxymethyl)-1-((2-(trimethylsilyl)ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)-methyl)piperazin-1-yl)phenyl)amino)piperidine-2,6-dione and 3-((3-chloro-4-(4-((1-(4-(1-((S)-1-(4-(2-(methoxy-methyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)phenyl)amino)piperidine-2,6-dione (0.17 g) was prepared similarly to Int. 2C18 from Ints. 2F8/2F9/2I9 (0.20 g/0.21 g, HCl salt).

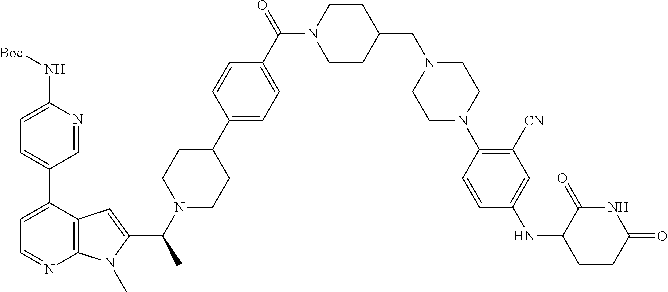 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5C#N)CC4)CC3)cc2)CC1]

Int. 2117. tert-butyl (5-(2-((1S)-1-(4-(4-(4-((4-(2-cyano-4-((2,6-dioxopiperidin-3-yl)amino)phenyl)-piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (0.15 g) was prepared similarly to Int. 2C18 from Ints. 3E76/2I13 (0.10 g/81 mg, HCl salt).

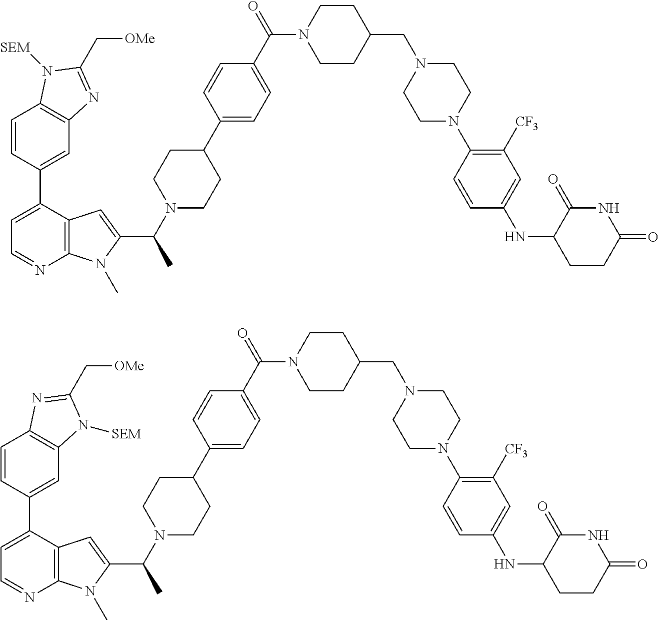 [SMILES: COCc1nc2cc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)cc8C(F)(F)F)CC7)CC6)cc5)CC3)n4C)cc2n1COCC[Si](C)(C)C]

Int. 2118 and 2119. 3-((4-(4-((1-(4-(1-((S)-1-(4-(2-(methoxymethyl)-1-((2-(trimethylsilyl)ethoxy)methyl)-1H-benzo[d]imidazol-5-yl)-1-methyl-1H-pyrrolo[2,3-b]pyridin-2-yl)ethyl)piperidin-4-yl)benzoyl)piperidin-4-yl)-methyl)piperazin-1-yl)-3-(trifluoromethyl)phenyl)amino)piperidine-2,6-dione and 3-((4-(4-((1-(4-(1-((S)-1-(4-(2-(methoxymethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)-3-(trifluoromethyl)phenyl)-amino)piperidine-2,6-dione (0.50 g) was prepared similarly to Int. 2C18 from Ints. 2F8/2F9/2I20 (0.20 g/0.58 g, HCl salt).

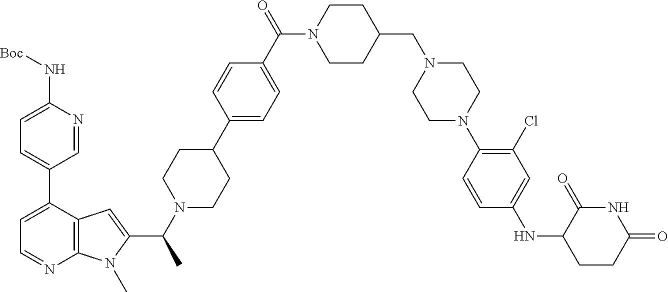 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5Cl)CC4)CC3)cc2)CC1]

Int. 2128. tert-butyl (5-(2-((1S)-1-(4-(4-(4-((4-(2-chloro-4-((2,6-dioxopiperidin-3-yl)amino)-phenyl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (60 mg) was prepared similarly to Int. 2C18 from Ints. 3E76/2I9 (0.10 g/0.16 g, HCl salt).

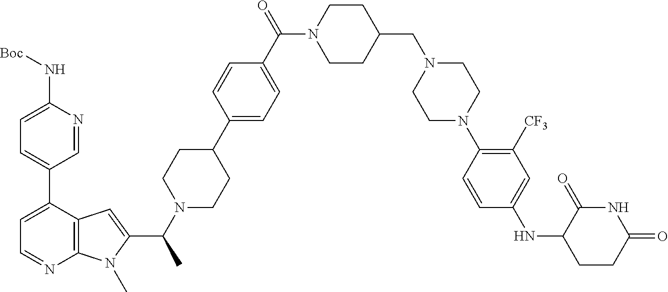 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)cc5C(F)(F)F)CC4)CC3)cc2)CC1]

Int. 2129. tert-butyl (5-(2-((1S)-1-(4-(4-(4-((4-(4-((2,6-dioxopiperidin-3-yl)amino)-2-(trifluoromethyl)phenyl)-piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (0.25 g) was prepared similarly to Int. 2C18 from Ints. 3E76/2I20 (0.15 g/0.51 g, HCl salt).

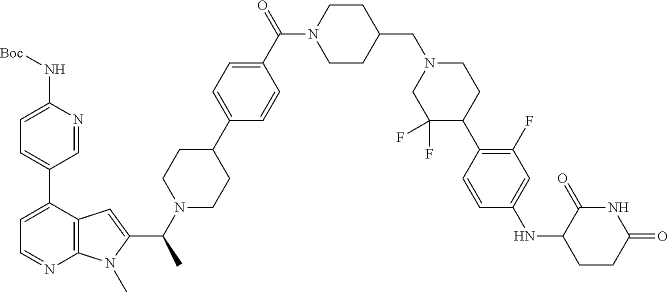 [SMILES: C[C@@H](c1cc2c(-c3ccc(NC(=O)OC(C)(C)C)nc3)ccnc2n1C)N1CCC(c2ccc(C(=O)N3CCC(CN4CCC(c5ccc(NC6CCC(=O)NC6=O)cc5F)C(F)(F)C4)CC3)cc2)CC1]

Int. 2M6. tert-butyl (5-(2-((1S)-1-(4-(4-(4-((4-(4-((2,6-dioxopiperidin-3-yl)amino)-2-fluorophenyl)-3,3-difluoropiperidin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (0.11 g) was prepared similarly to Int. 2C18 from Ints. 3E76/2M7 (85 mg/0.12 g, HCl salt).

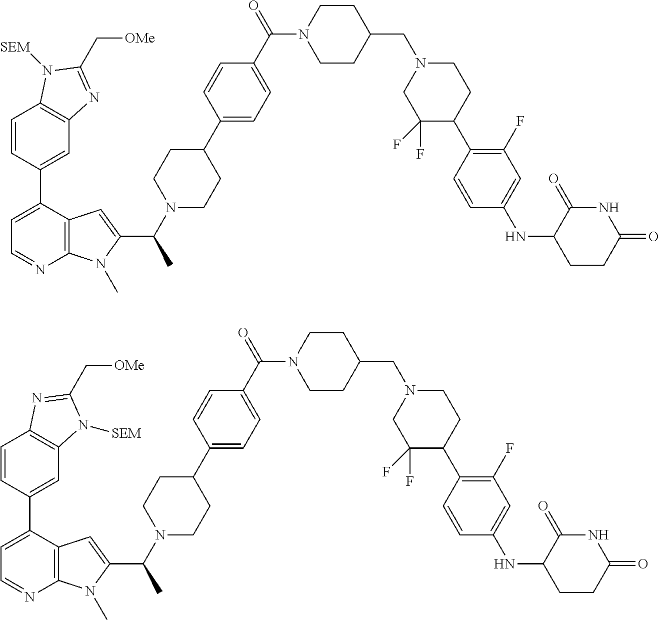 [SMILES: COCc1nc2cc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCC(c8ccc(NC9CCC(=O)NC9=O)cc8F)C(F)(F)C7)CC6)cc5)CC3)n4C)cc2n1COCC[Si](C)(C)C]

Int. 2M13 and 2M14. 3-((4-(3,3-difluoro-1-((1-(4-(1-((S)-1-(4-(2-(methoxymethyl)-1-((2-(trimethylsilyl)ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperidin-4-yl)-3-fluorophenyl)amino)piperidine-2,6-dione and 3-((4-(3,3-difluoro-1-((1-(4-(1-((S)-1-(4-(2-(methoxymethyl)-1-((2-(trimethylsilyl)ethoxy)-methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperidin-4-yl)-3-fluorophenyl)-amino)piperidine-2,6-dione (0.20 g) was prepared similarly to Int. 2C18 from Ints. 2F8/2F9/2M7 (0.20 g/0.24 g, HCl salt).

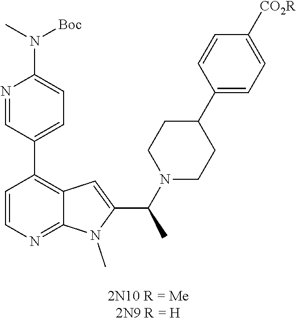

Int. 3E35 (50 mg), tert-butyl methyl(5-(4,4,5,5-tetramethyl-1,3,2-dioxaborolan-2-yl)pyridin-2-yl)carbamate (37 mg), NaHCO₃ (18 mg), and PdDPPFCl₂-DCM (9 mg) were degassed in dioxane/water (9:1, 2 mL) and stirred ON at 120° C. The OL (EtOAc/water) was dried, concentrated, and purified by FC (pentane/EtOAc 9:1 to 7:3) to give Int. 2N10 methyl 4-[1-[(1S)-1-[4-[6-[tert-butoxycarbonyl(methyl)-amino]-3-pyridyl]-1-methyl-pyrrolo[2,3-b]pyridin-2-yl]ethyl]-4-piperidyl]-benzoate (30 mg). LiOH—H₂O (76 mg) and Int. 2N10 (0.35 g) were stirred 1 h at 0° C. to RT in THF/MeOH/water (3:3:1, 5 mL) and neutralized with 1M aq. HCl to precipitate Int. 2N9 (S)-4-(1-(1-(4-(6-((tert-butoxycarbonyl)(methyl)amino)pyridin-3-yl)-1-methyl-1H-pyrrolo[2,3-b]pyridin-2-yl)ethyl)piperidin-4-yl)benzoic acid (0.21 g).

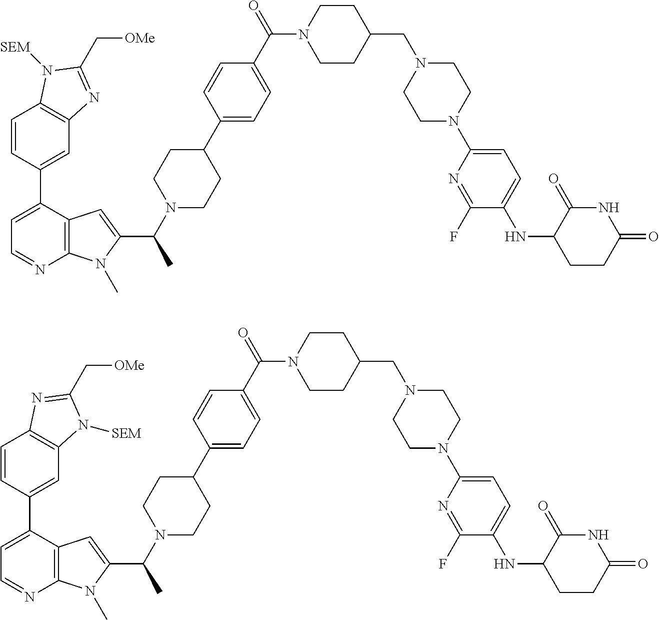 [SMILES: COCc1nc2cc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ccc(NC9CCC(=O)NC9=O)c(F)n8)CC7)CC6)cc5)CC3)n4C)cc2n1COCC[Si](C)(C)C]

Int. 2N11 and 2N12. 3-((2-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((2-fluoro-6-(4-((1-(4-(1-((S)-1-(4-(2-(methoxymethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)-amino)piperidine-2,6-dione (80 mg) was prepared similarly to Int. 2C18 from Ints. 2F8/2F9/2N14 (0.10 g/88 mg, HCl salt).

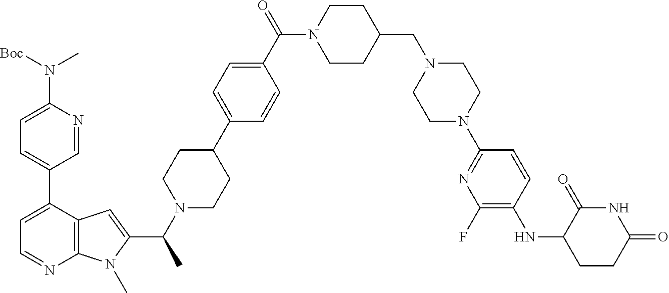 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

Int. 2N13. tert-butyl (5-(2-((1S)-1-(4-(4-(4-((4-(5-((2,6-dioxopiperidin-3-yl)amino)-6-fluoropyridin-2-yl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)(methyl)carbamate (0.20 g) was prepared similarly to Int. 2C18 from Int. 2N9/2N14 (0.20 g/0.22 g, HCl salt).

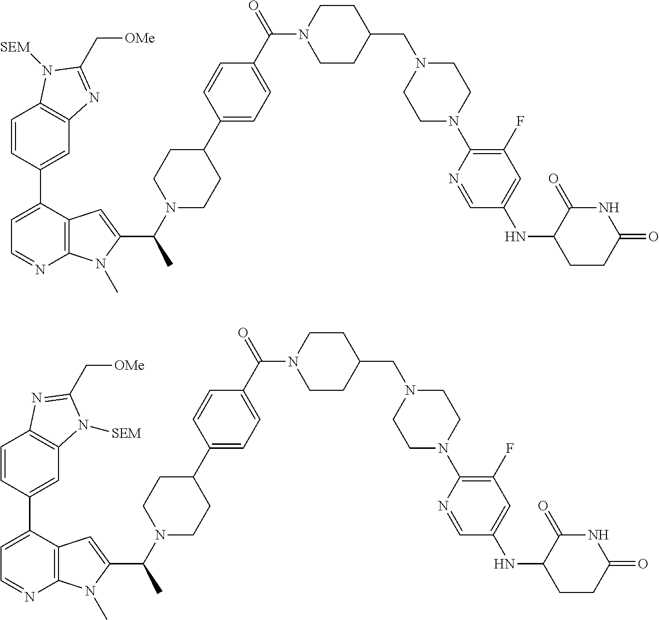 [SMILES: COCc1nc2cc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)ccc2n1COCC[Si](C)(C)C ; COCc1nc2ccc(-c3ccnc4c3cc([C@H](C)N3CCC(c5ccc(C(=O)N6CCC(CN7CCN(c8ncc(NC9CCC(=O)NC9=O)cc8F)CC7)CC6)cc5)CC3)n4C)cc2n1COCC[Si](C)(C)C]

Int. 2N18 and 2N19. 3-((5-fluoro-6-(4-((1-(4-(1-((S)-1-(4-(2-(methoxymethyl)-1-((2-(trimethylsilyl)-ethoxy)-methyl)-1H-benzo[d]imidazol-5-yl)-1-methyl-1H-pyrrolo[2,3-b]pyridin-2-yl)ethyl)piperidin-4-yl)benzoyl)-piperidin-4-yl)methyl)piperazin-1-yl)pyridin-3-yl)amino)piperidine-2,6-dione and 3-((5-fluoro-6-(4-((1-(4-(1-((S)-1-(4-(2-(methoxymethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)-piperidine-2,6-dione (0.10 g) was prepared similarly to Int. 2C18 from Ints. 2F8/2F9/2N5 (80 mg/76 mg, HCl salt).

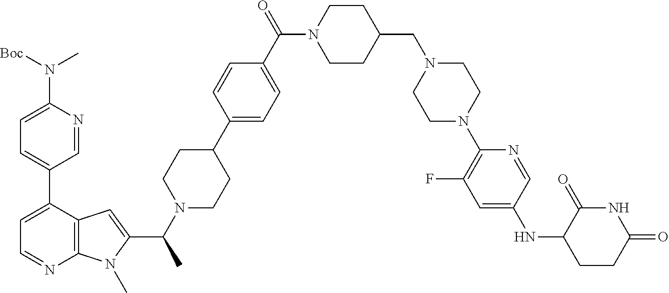 [SMILES: C[C@@H](c1cc2c(-c3ccc(N(C)C(=O)OC(C)(C)C)nc3)ccnc2n1C)N1CCC(c2ccc(C(=O)N3CCC(CN4CCN(c5ncc(NC6CCC(=O)NC6=O)cc5F)CC4)CC3)cc2)CC1]

Int. 2N20. tert-butyl (5-(2-((1S)-1-(4-(4-(4-((4-(5-((2,6-dioxopiperidin-3-yl)amino)-3-fluoropyridin-2-yl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)(methyl)carbamate (0.20 g) was prepared similarly to Int. 2C18 from Ints. 2N9/2N5 (0.15 g/0.16 g, HCl salt).

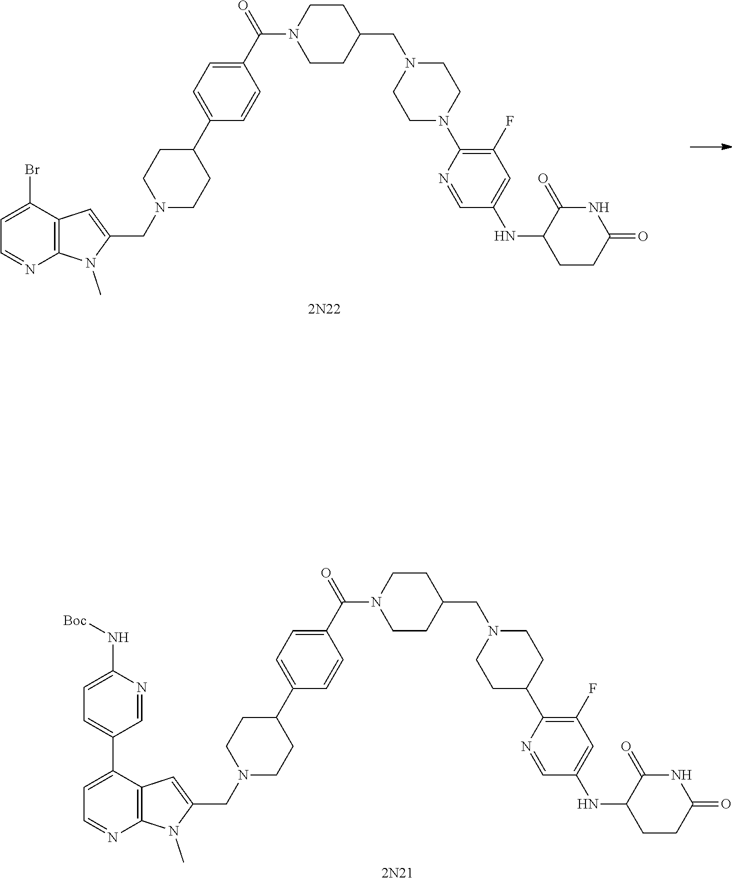 [SMILES: Cn1c(CN2CCC(c3ccc(C(=O)N4CCC(CN5CCN(c6ncc(NC7CCC(=O)NC7=O)cc6F)CC5)CC4)cc3)CC2)cc2c(Br)ccnc21 ; Cn1c(CN2CCC(c3ccc(C(=O)N4CCC(CN5CCC(c6ncc(NC7CCC(=O)NC7=O)cc6F)CC5)CC4)cc3)CC2)cc2c(-c3ccc(NC(=O)OC(C)(C)C)nc3)ccnc21]

Int. 2N22 3-((6-(4-((1-(4-(1-((4-bromo-1-methyl-1H-pyrrolo[2,3-b]pyridin-2-yl)methyl)-piperidin-4-yl)benzoyl)-piperidin-4-yl)methyl)piperazin-1-yl)-5-fluoropyridin-3-yl)amino)piperidine-2,6-dione (57 mg) was prepared similarly to Int. 2C18 from Ints. 1AC4/2N5 (70 mg/86 mg, HCl salt). tert-Butyl (5-(4,4,5,5-tetramethyl-1,3,2-dioxaborolan-2-yl)pyridin-2-yl)-carbamate (29 mg), Int. 2N22 (53 mg), and PdDPPFCl₂-DCM (5 mg) were degassed in DMF/10% aq. Na₂CO₃ (5:1, 0.84 mL) and stirred 0.5 h at 100° C. The OL (EtOAc/water) was washed with brine, dried, concentrated, and purified by FC (DCM/MeOH 1:0 to 9:1) to give Int. 2N21 tert-butyl (5-(2-((4-(4-(4-((4-(5-((2,6-dioxopiperidin-3-yl)amino)-3-fluoropyridin-2-yl)piperazin-1-yl)-methyl)piperidine-1-carbonyl)phenyl)piperidin-1-yl)methyl)-1-methyl-1H-pyrrolo[2,3-b]pyridin-4-yl)pyridin-2-yl)carbamate (20 mg).

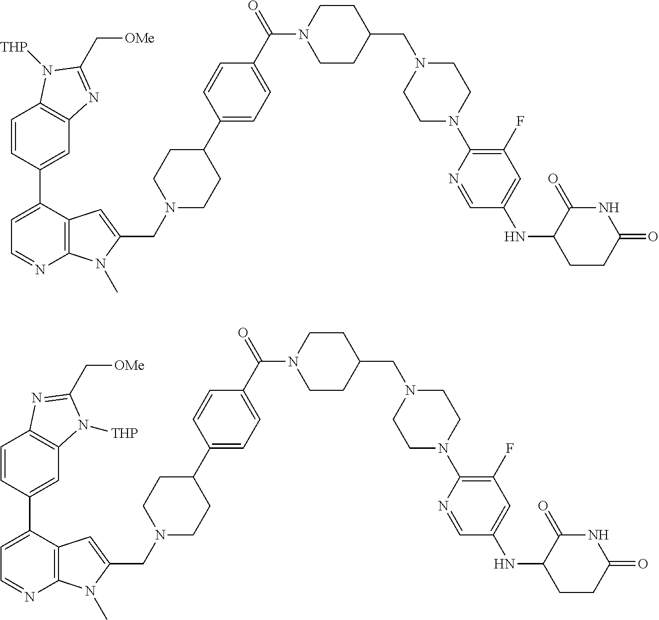

Int. 2N23 and 2N24. 3-((5-fluoro-6-(4-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione and 3-((5-fluoro-6-(4-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoyl)piperidin-4-yl)methyl)piperazin-1-yl)pyridin-3-yl)amino)piperidine-2,6-dione (15 mg) was prepared similarly to Int. 2C18 from Ints. 2C55/2N5 (29 mg/21 mg, HCl salt).

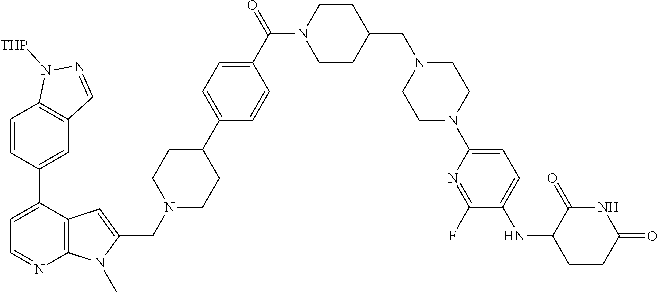

Int. 2N25. 3-((2-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione and 3-((2-fluoro-6-(4-((1-(4-(1-((1S)-1-(4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (106 mg) was prepared similarly to Int. 2C18 from Int. 2C54 (92 mg) and 2N14 (90 mg, HCl salt).

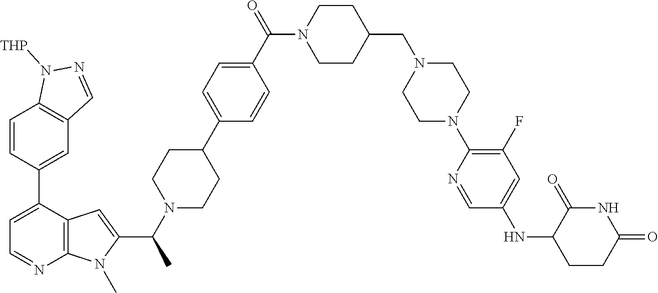

Int. 2N26. 3-((5-fluoro-6-(4-((1-(4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoyl)piperidin-4-yl)methyl)piperazin-1-yl)pyridin-3-yl)amino)piperidine-2,6-dione (63 mg) was prepared similarly to Int. 2C18 from Ints. 2C54/2N5 (53 mg/52 mg, HCl salt).

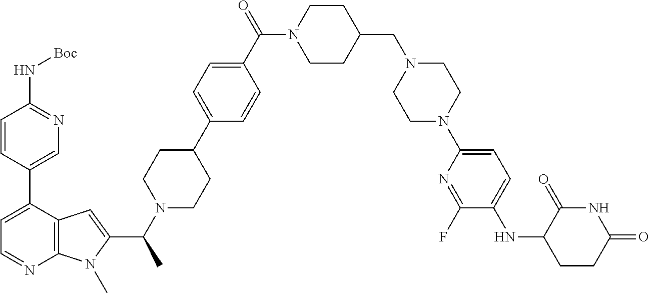 [SMILES: C[C@@H](c1cc2c(-c3ccc(NC(=O)OC(C)(C)C)nc3)ccnc2n1C)N1CCC(c2ccc(C(=O)N3CCC(CN4CCN(c5ccc(NC6CCC(=O)NC6=O)c(F)n5)CC4)CC3)cc2)CC1]

Int. 2N27. tert-butyl (5-(2-((1S)-1-(4-(4-(4-((4-(5-((2,6-dioxopiperidin-3-yl)amino)-6-fluoropyridin-2-yl)piperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (104 mg) was prepared similarly to Int. 2C18 from Ints. 3E76/2N14 (90 mg/89 mg, HCl salt).

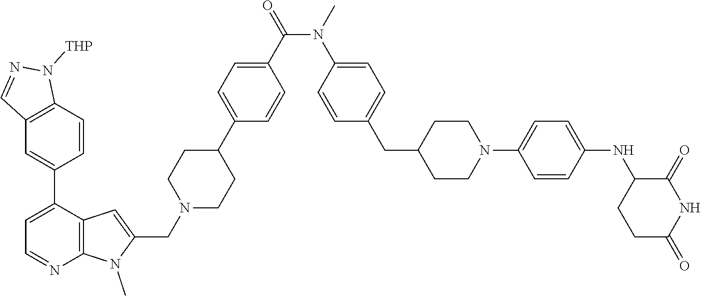

Int. 201 (50 mg), 1-(tetrahydro-2H-pyran-2-yl)-5-(4,4,5,5-tetramethyl-1,3,2-dioxaborolan-2-yl)-1H-indazole (19 mg), and PdDPPFCl₂-DCM (4 mg) were degassed in DMF/10% aq. Na₂CO₃ (15:1, 1.1 mL) and stirred 1.5 h at 100° C. The filtrate after filtration was purified by FC (DCM/MeOH 1:0 to 9:1) to give Int. 206 N-(4-((1-(4-((2,6-dioxopiperidin-3-yl)amino)phenyl)piperidin-4-yl)methyl)phenyl)-N-methyl-4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzamide (46 mg).

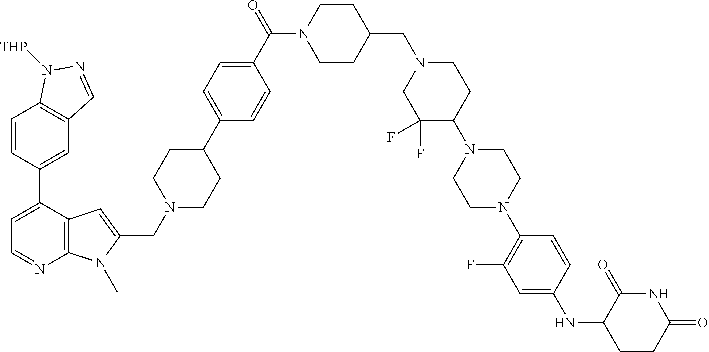

Int. 2P1. 3-((4-(4-(3,3-difluoro-1-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperidin-4-yl)piperazin-1-yl)-3-fluorophenyl)amino)piperidine-2,6-dione (25 mg) was prepared similarly to Int. 2C18 from HCl salts of Ints. 3A2/2P2 (25 mg) and 2P2 (22 mg).

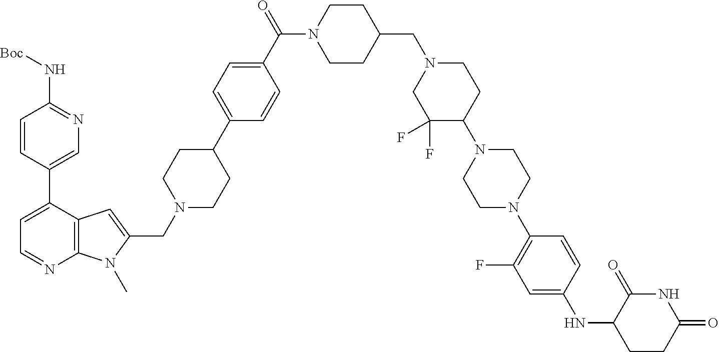 [SMILES: Cn1c(CN2CCC(c3ccc(C(=O)N4CCC(CN5CCC(N6CCN(c7ccc(NC8CCC(=O)NC8=O)cc7F)CC6)C(F)(F)C5)CC4)cc3)CC2)cc2c(-c3ccc(NC(=O)OC(C)(C)C)nc3)ccnc21]

Int. 2P9. tert-butyl (5-(2-((4-(4-(4-((4-(4-(4-((2,6-dioxopiperidin-3-yl)amino)-2-fluorophenyl)piperazin-1-yl)-3,3-difluoropiperidin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (27 mg) was prepared similarly to Int. 2C18 from Int. 2E6 (20 mg, HCl salt) and 2P2 (22 mg, HCl salt).

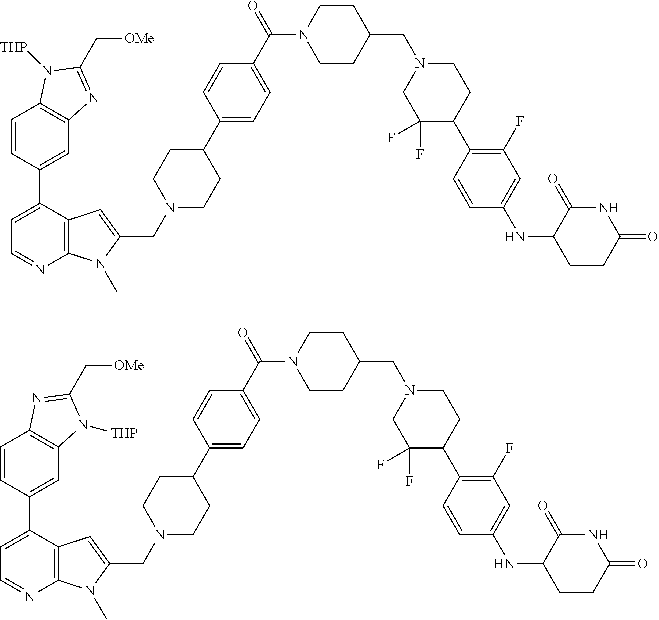

Int. 2P10 and 2P11. 3-((4-(4-(3,3-difluoro-1-((1-(4-(1-((4-(2-(methoxymethyl)-1-(tetrahydro-2H-pyran-2-yl)-1H-benzo[d]imidazol-5-yl)-1-methyl-1H-pyrrolo[2,3-b]pyridin-2-yl)methyl)piperidin-4-yl)benzoyl)-piperidin-4-yl)-methyl)piperidin-4-yl)piperazin-1-yl)-3-fluorophenyl)amino)piperidine-2,6-dione and 3-((4-(3,3-difluoro-1-((1-(4-(1-((4-(2-(methoxymethyl)-1-(tetrahydro-2H-pyran-2-yl)-1H-benzo[d]imidazol-6-yl)-1-methyl-1H-pyrrolo[2,3-b]pyridin-2-yl)methyl)piperidin-4-yl)benzoyl)piperidin-4-yl)methyl)piperidin-4-yl)-3-fluorophenyl)amino)-piperidine-2,6-dione (20 mg) was prepared similarly to Int. 2C18 from Ints. 2C55/2P2 (18 mg/19 mg, HCl salt).

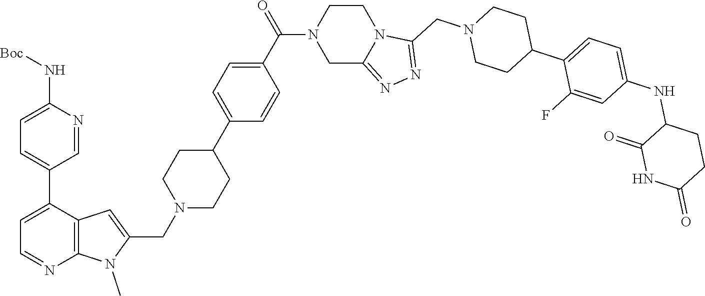 [SMILES: Cn1c(CN2CCC(c3ccc(C(=O)N4CCn5c(CN6CCC(c7ccc(NC8CCC(=O)NC8=O)cc7F)CC6)nnc5C4)cc3)CC2)cc2c(-c3ccc(NC(=O)OC(C)(C)C)nc3)ccnc21]

Int. 2Q1. tert-butyl (5-(2-((4-(4-(3-((4-(4-((2,6-dioxopiperidin-3-yl)amino)-2-fluorophenyl)-piperidin-1-yl)methyl)-5,6,7,8-tetrahydro-[1,2,4]triazolo[4,3-a]pyrazine-7-carbonyl)phenyl)piperidin-1-yl)methyl)-1-methyl-1H-pyrrolo[2,3-b]pyridin-4-yl)pyridin-2-yl)carbamate (80 mg) was prepared similarly to Int. 2C18 from Ints. 2E6/2Q2 (44 mg/55 mg, HCl salt).

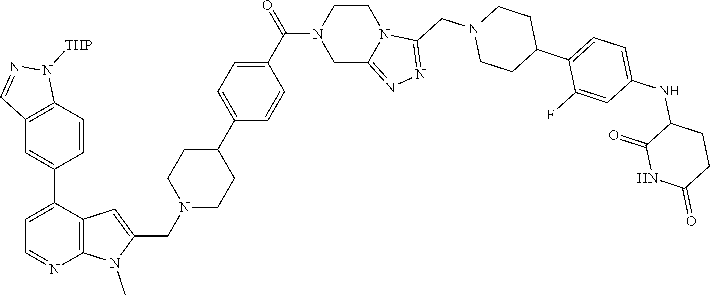

Int. 2Q9. 3-((3-fluoro-4-(1-((7-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-5,6,7,8-tetrahydro-[1,2,4]triazolo[4,3-a]pyrazin-3-yl)methyl)-piperidin-4-yl)phenyl)amino)-piperidine-2,6-dione (0.12 g) was prepared similarly to Int. 2C18 from Ints. 3A2 (44 mg) and 2Q2 (55 mg, HCl salt).

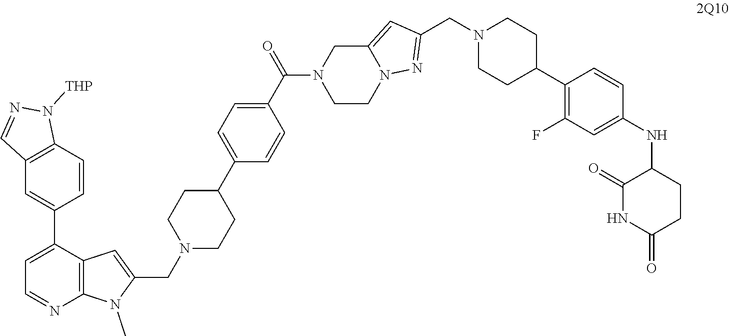

Int. 2Q10 3-((3-fluoro-4-(1-((5-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)-4,5,6,7-tetrahydropyrazolo[1,5-a]pyrazin-2-yl)methyl)piperidin-4-yl)phenyl)amino)piperidine-2,6-dione (60 mg) was prepared similarly to Int. 2C18 from Ints. 3A2/2Q15 (46 mg/40 mg, HCl salt).

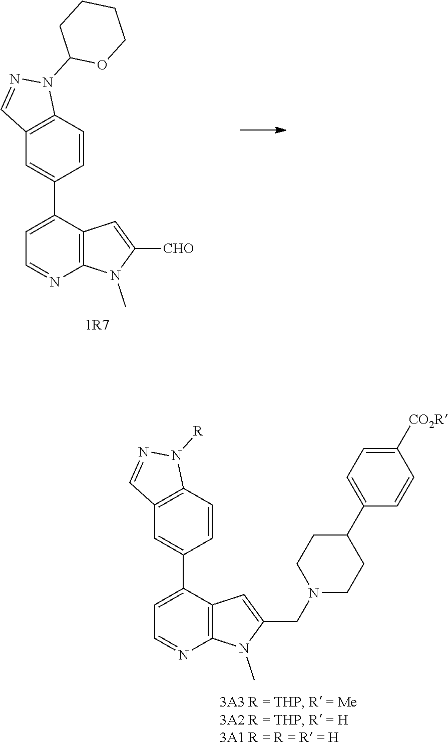

Methyl 4-(4-piperidyl)benzoate (0.13 g) and Int. 1R7 (0.11 g) were stirred 0.75 h in DCE (4 mL). STAB (0.19 g) was added and stirring continued 4 h. The OL (DCM/sat. aq. NaHCO₃) was concentrated and HPLC-purified to give Int. 3A3 methyl 4-(1-((1-methyl-4-(1-(tetrahydro-2H-pyran-2-yl)-1H-indazol-5-yl)-1H-pyrrolo[2,3-b]-pyridin-2-yl)-methyl)-piperidin-4-yl)benzoate (0.12 g). Int. 3A3 (2.0 g) was stirred ON in THF/MeOH/2M aq. NaOH (2:1:1; 22 mL). 2M aq. NaOH (2 mL) was added and stirring continued 0.5 h. The mixture was neutralized with aq. HCl and concentrated to give Int. 3A2 4-(1-((1-methyl-4-(1-(tetrahydro-2H-pyran-2-yl)-1H-indazol-5-yl)-1H-pyrrolo[2,3-b]pyridin-2-yl)methyl)-piperidin-4-yl)benzoic acid (1.95 g, HCl salt). 1.0 g of this material was stirred 0.75 h in DCM/TFA (2:1; 3 mL). The residue after concentration was triturated in MeOH to give Int. 3A1 4-[1-[[4-(1H-indazol-5-yl)-1-methyl-pyrrolo[2,3-b]pyridin-2-yl]methyl]-4-piperidyl]benzoic acid (0.62 g).

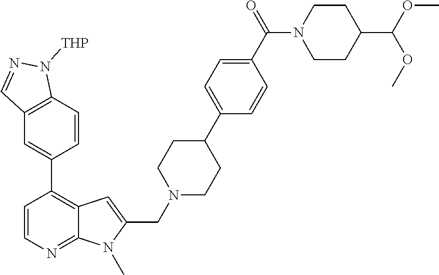

Int. 3A2 (0.50 g, HCl salt) and HATU (0.49 g) were stirred 0.3 h in DMF/DIPEA (20:1, 10.5 mL). 4-(Dimethoxy-methyl)piperidine (0.16 g) was added and stirring continued ON. The OL (water/EtOAc) was washed with brine, dried, concentrated, and purified by FC (DCM/MeOH 1:0 to 95:5) to give Int. 3D4 (4-(dimethoxy-methyl)-piperidin-1-yl)(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)phenyl)methanone (0.20 g).

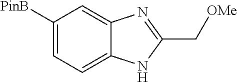

5-Bromo-2-(methoxymethyl)-1H-benzo[d]imidazole (2.0 g), B₂Pin₂ (5.3 g), KOAc (2.4 g), and PdDPPFCl₂-DCM (0.34 g) were degassed in dioxane (20 mL) and stirred ON at 100° C., concentrated, and purified by FC (hexane/EtOAc 1:4) to give Int. 3E8 2-(methoxymethyl)-5-(4,4,5,5-tetramethyl-1,3,2-dioxaborolan-2-yl)-1H-benzo[d]-imidazole (2.1 g).

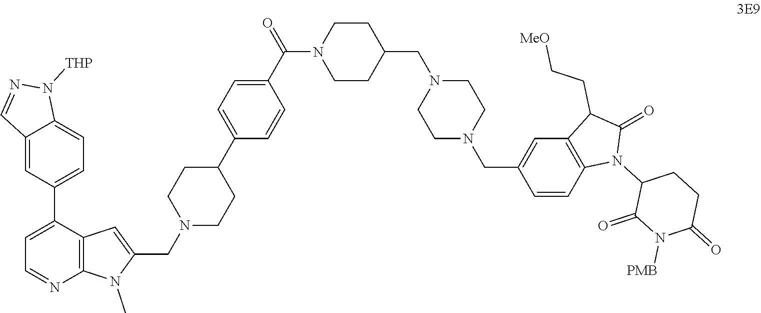

Int. 3E12 (30 mg), Int. 3A2 (34 mg), and HATU (24 mg) were stirred 2 h in DMF/DIPEA (23.8:1, 2.1 mL), filtered, and HPLC-purified to give Int. 3E9 1-(4-methoxybenzyl)-3-(3-(2-methoxyethyl)-5-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)piperazin-1-yl)methyl)-2-oxo-2,3-dihydro-1H-benzo[d]imidazol-1-yl)piperidine-2,6-dione (51 mg).

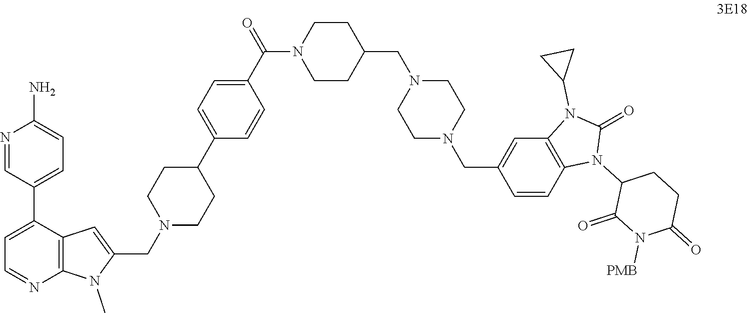

Int. 3E18 3-(5-((4-((1-(4-(1-((4-(6-aminopyridin-3-yl)-1-methyl-1H-pyrrolo[2,3-b]pyridin-2-yl)methyl)piperidin-4-yl)benzoyl)piperidin-4-yl)methyl)piperazin-1-yl)methyl)-3-cyclo-propyl-2-oxo-2,3-dihydro-1H-benzo[d]imidazol-1-yl)-1-(4-methoxybenzyl)piperidine-2,6-dione (33 mg) was prepared similarly to Int. 3E9 from Ints. 3E23/3E19 (29 mg/36 mg).

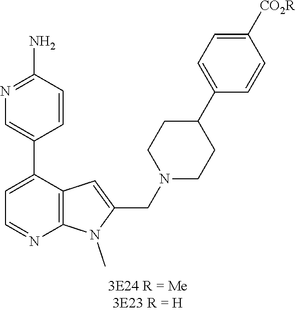

Int. 1AC5 (0.50 g), 5-(4,4,5,5-tetramethyl-1,3,2-dioxaborolan-2-yl)pyridin-2-amine (0.37 g), Na₂CO₃ (2.4 g), and PdDPPFCl₂-DCM (92 mg) were degassed in DMF/water (5.7:1, 6.7 mL) and stirred 1 h at 100° C. Filtration, concentration, and purification by FC (heptane/EtOAc 1:2 to 0:1) gave to give Int. 3E24 methyl 4-(1-((4-(6-aminopyridin-3-yl)-1-methyl-1H-pyrrolo[2,3-b]pyridin-2-yl)methyl)piperidin-4-yl)benzoate (0.39 g). Int. 3E24 (1.0 g) was stirred 5 h in THF/MeOH (2:1; 39 mL) and 2M aq. NaOH (5.7 mL). The mixture was acidified with 5M aq. HCl (pH 1-2), neutralized with 1M aq. LiOH, concentrated, and HPLC-purified to give Int. 3E23 4-(1-((4-(6-aminopyridin-3-yl)-1-methyl-1H-pyrrolo[2,3-b]pyridin-2-yl)methyl)piperidin-4-yl)benzoic acid (0.42 g).

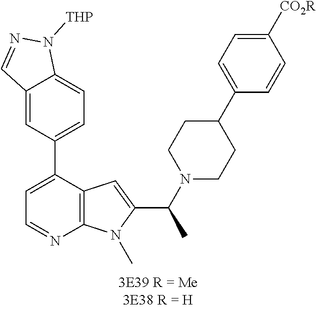

Int. 3E35 (60 mg), 1-(tetrahydro-2H-pyran-2-yl)-5-(4,4,5,5-tetramethyl-1,3,2-dioxaborolan-2-yl)-1H-indazole (60 mg), Na₂CO₃ (0.28 mL of a 10% aq. solution), and PdDPPFCl₂-DCM (11 mg) were degassed in DMF (0.8 mL) and stirred 1 h at 100° C. The OL (aq. NaHCO₃/EtOAc) was washed with brine, dried, concentrated, and purified by FC (heptane/EtOAc 1:2) to give Int. 3E39 methyl 4-(1-((1S)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoate or methyl 4-(1-((1R)-1-(1-methyl-4-(1-(tetra-hydro-2H-pyran-2-yl)-1H-indazol-5-yl)-1H-pyrrolo[2,3-b]pyridin-2-yl)-ethyl)piperidin-4-yl)benzoate (63 mg). Int. 3E39 (59 mg) was stirred in THF/MeOH/1M aq. NaOH (1.3:2.8:1, 2 mL) at 0° C. to RT ON. The mixture was partially concentrated. The OL (brine/2M aq. HCl/DCM:MeOH (9:1)) was dried, concentrated, and purified by FC (DCM/MeOH 95:5) to give 4-(1-((1S)-1-(1-methyl-4-(1-(tetra-hydro-2H-pyran-2-yl)-1H-indazol-5-yl)-1H-pyrrolo-[2,3-b]pyridin-2-yl)ethyl)piperidin-4-yl)benzoic acid or 4-(1-((1R)-1-(1-methyl-4-(1-(tetrahydro-2H-pyran-2-yl)-1H-indazol-5-yl)-1H-pyrrolo[2,3-b]pyridin-2-yl)ethyl)piperidin-4-yl)benzoic acid (47 mg). 43 mg of this material was stirred 1.5 h in DCM/TFA (4:1; 1 mL) and concentrated to give Int. 3E38 (S)-4-(1-(1-(4-(1H-indazol-5-yl)-1-methyl-1H-pyrrolo[2,3-b]pyridin-2-yl)ethyl)piperidin-4-yl)benzoic acid or (R)-4-(1-(1-(4-(1H-indazol-5-yl)-1-methyl-1H-pyrrolo[2,3-b]pyridin-2-yl)ethyl)piperidin-4-yl)benzoic acid (64 mg).

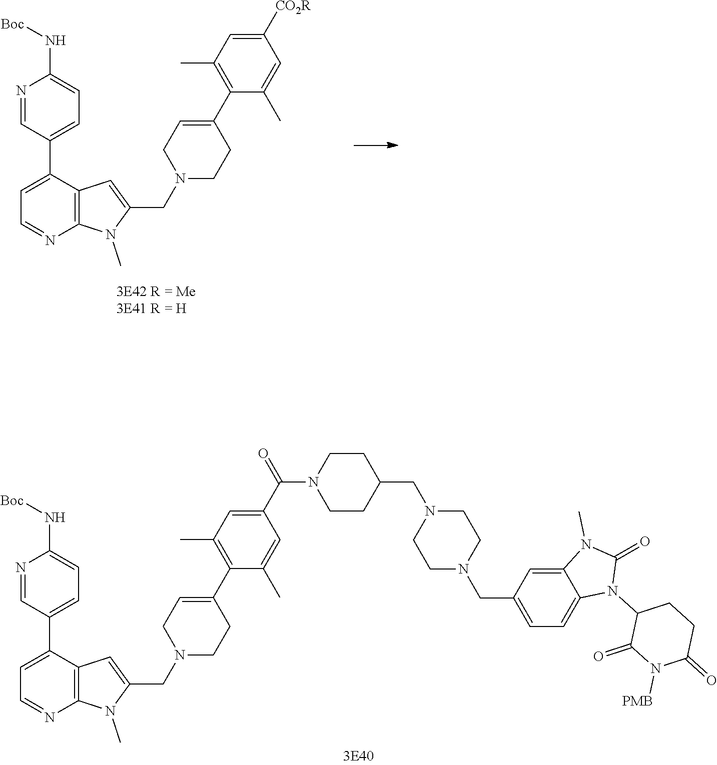

Int. 3E43 (0.32 g), tert-butyl (5-(4,4,5,5-tetramethyl-1,3,2-dioxaborolan-2-yl)pyridin-2-yl)carbamate (0.24 g), NaHCO₃ (86 mg), and PdDPPFCl₂-DCM (28 mg) were degassed in dioxane/water (9:1; 8 mL) and stirred ON at 90° C. The OL (water/EtOAc) was concentrated and purified by FC (hexane/EtOAc 7:3) to give Int. 3E42 methyl 4-[1-[[4-[6-(tert-butoxycarbonylamino)-3-pyridyl]-1-methyl-pyrrolo[2,3-b]pyridin-2-yl]methyl]-3,6-dihydro-2H-pyridin-4-yl]-3,5-dimethyl-benzoate (0.14 g). Int. 3E42 (0.82 g) and LiOH—H₂O (0.30 g) were stirred in THF/MeOH/water (6:2:1; 7 mL) ON. The mixture was neutralized with 0.5M aq. HCl to precipitate Int. 3E41 4-(1-((4-(6-((tert-butoxy-carbonyl)amino)-pyridin-3-yl)-1-methyl-1H-pyrrolo[2,3-b]pyridin-2-yl)methyl)-1,2,3,6-tetrahydropyridin-4-yl)-3,5-dimethylbenzoic acid (0.45 g). Int. 3E2 (30 mg) was stirred 0.5 h in DCM/TFA (4:1; 1 mL) and concentrated. The residue, Int. 3E41 (22 mg), and HATU (18 mg) were stirred 0.75 h in DCM/DIPEA (8.6:1, 0.67 mL). The OL (sat. aq. NaHCO₃/DCM) was washed with brine, dried, concentrated, and purified by FC (DCM/MeOH 96:4) to give Int. 3E40 tert-butyl (5-(2-((4-(4-(4-((4-((1-(1-(4-methoxybenzyl)-2,6-dioxopiperidin-3-yl)-3-methyl-2-oxo-2,3-dihydro-1H-benzo[d]imidazol-5-yl)methyl)piperazin-1-yl)methyl)piperidine-1-carbonyl)-2,6-dimethylphenyl)-3,6-dihydropyridin-1(2H)-yl)methyl)-1-methyl-1H-pyrrolo[2,3-b]pyridin-4-yl)pyridin-2-yl)carbamate (38 mg).

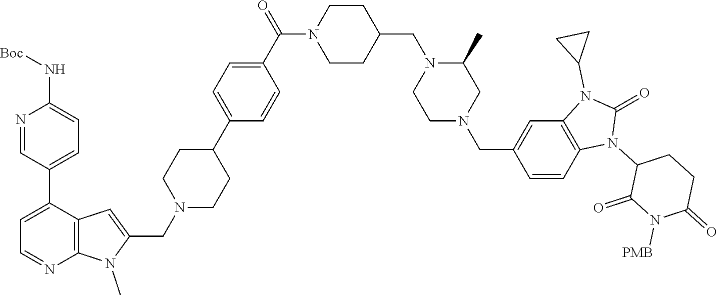

Int. 3E32 (0.22 g), PdDPPFCl₂-DCM (18 mg), Na₂CO₃ (0.46 mL of a 10% aq. solution), and tert-butyl (5-(4,4,5,5-tetramethyl-1,3,2-dioxaborolan-2-yl)pyridin-2-yl)carbamate (0.11 g) were degassed in DMF (2.2 mL) and stirred 2 h at 100° C. Filtration, concentration, and purification by FC (DCM/MeOH 95:5 t 9:1) gave Int. 3E46 tert-butyl (5-(2-((4-(4-(4-(((2S)-4-((3-cyclo-propyl-1-(1-(4-methoxybenzyl)-2,6-dioxopiperidin-3-yl)-2-oxo-2,3-dihydro-1H-benzo[d]imidazol-5-yl)methyl)-2-methylpiperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (87 mg).

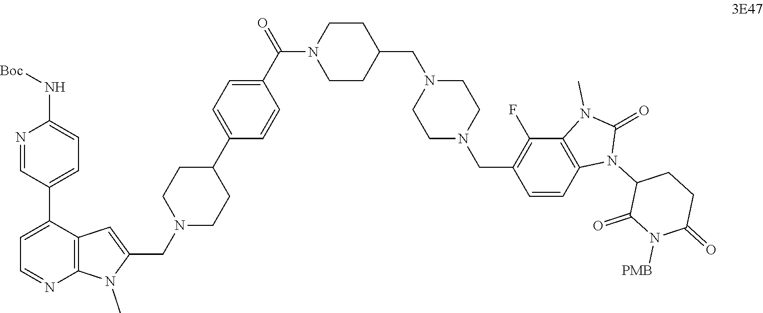

Int. 3E48 (0.10 g, TFA salt), Int. 3E56 (66 mg), and HATU (52 mg) were stirred ON in DMF/DIPEA (10:1, 2.2 mL). The OL (water/EtOAc) was washed with brine, dried, concentrated, and purified by FC (DCM/MeOH 1:0 to 9:1) to give Int. 3E47 tert-butyl (5-(2-((4-(4-(4-((4-((4-fluoro-1-(1-(4-methoxybenzyl)-2,6-dioxopiperidin-3-yl)-3-methyl-2-oxo-2,3-dihydro-1H-benzo[d]imidazol-5-yl)methyl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (95 mg).

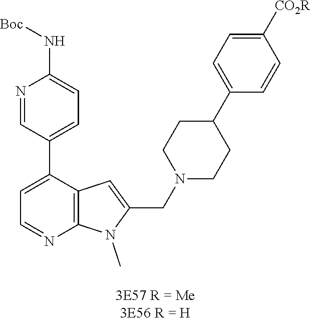

Int. 1AC5 (1.0 g), tert-butyl (5-(4,4,5,5-tetramethyl-1,3,2-dioxaborolan-2-yl)pyridin-2-yl)carbamate (0.8 g), NaHCO₃ (0.38 g), and PdDPPFCl₂-DCM (0.18 g) were degassed in dioxane/water (5:1; 12 mL) and stirred ON at 100° C., filtered, and concentrated. The residue was combined with three other batches prepared similarly and purified by FC (pentane/EtOAc 1:0 to 3:2) to give Int. 3E57 methyl 4-(1-((4-(6-((tert-butoxy-carbonyl)amino)-pyridin-3-yl)-1-methyl-1H-pyrrolo[2,3-b]pyridin-2-yl)methyl)piperidin-4-yl)benzoate (3.5 g). 1.8 g of this material and LiOH—H₂O (0.65 g) were stirred ON in MeOH/THF/water (2:2:1; 25 mL) at 0° C. to RT, concentrated, and triturated in dilute aq. HCl to give Int. 3E56 4-(1-((4-(6-((tert-butoxycarbonyl)amino)pyridin-3-yl)-1-methyl-1H-pyrrolo[2,3-b]pyridin-2-yl)methyl)piperidin-4-yl)benzoic acid (1.2 g, HCl salt).

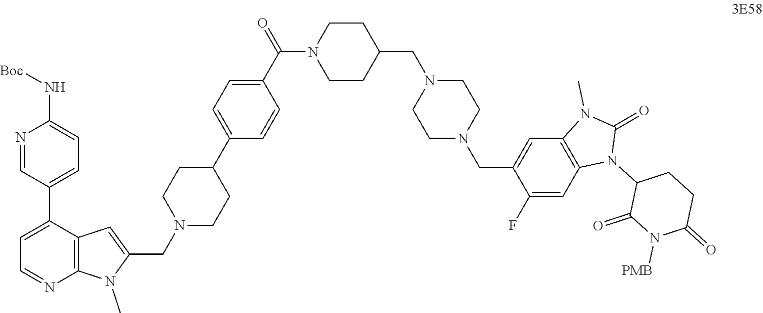

Int. 3E60 (41 mg) was stirred 1 h in DCM/TFA (1:1; 2 mL) and concentrated. The residue, Int. 3E56 (39 mg) and HATU (28 mg) were stirred ON in DMF/DIPEA (20:1, 2.1 mL), filtered, and HPLC-purified to give Int. 3E58 tert-butyl (5-(2-((4-(4-(4-((4-((6-fluoro-1-(1-(4-methoxybenzyl)-2,6-dioxopiperidin-3-yl)-3-methyl-2-oxo-2,3-dihydro-1H-benzo[d]-imidazol-5-yl)methyl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (51 mg).

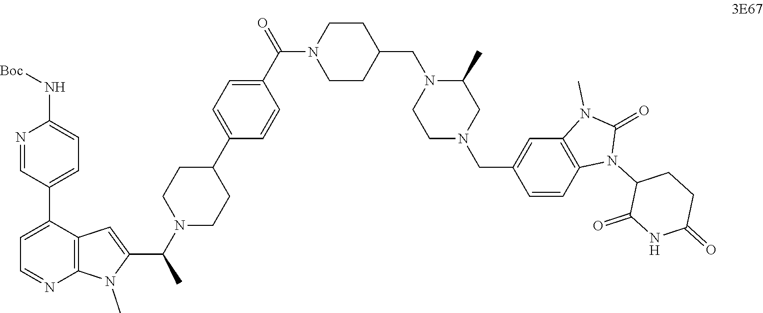 [SMILES: C[C@H]1CN(Cc2ccc3c(c2)n(C)c(=O)n3C2CCC(=O)NC2=O)CCN1CC1CCN(C(=O)c2ccc(C3CCN([C@@H](C)c4cc5c(-c6ccc(NC(=O)OC(C)(C)C)nc6)ccnc5n4C)CC3)cc2)CC1]

Ints. 3E76/3E68 (0.50 g/0.61 g) and HATU (0.44 g) were stirred ON in DMF/DIPEA (6.4:1, 5.8 mL) and diluted with water to precipitate Int. 3E67 tert-Butyl (5-(2-((1S)-1-(4-(4-(4-(((2S)-4-((1-(2,6-dioxopiperidin-3-yl)-3-methyl-2-oxo-2,3-dihydro-1H-benzo[d]imidazol-5-yl)methyl)-2-methylpiperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (0.52 g).

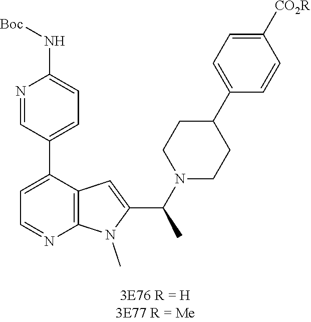

PdDPPFCl₂-DCM (0.8 g), tert-butyl (5-(4,4,5,5-tetramethyl-1,3,2-dioxaborolan-2-yl)pyridin-2-yl)carbamate (4.1 g), NaHCO₃ (1.7 g), and Int. 3E35 (4.5 g) were degassed in dioxane/water (5:2; 12 mL) and stirred ON at 80° C. The OL (water/EtOAc) was washed with brine, dried, concentrated, and purified by FC (pentane/EtOAc 7:3 to 3:2) to give Int. 3E77 methyl (S)-4-(1-(1-(4-(6-((tert-butoxycarbonyl)amino)pyridin-3-yl)-1-methyl-1H-pyrrolo-[2,3-b]-pyridin-2-yl)ethyl)piperidin-4-yl)benzoate (3.5 g). This material and LiOH—H₂O (1.3 g) were stirred ON in THF/MeOH/water (2:2:1; 37 mL), concentrated, and triturated in dilute aq. citric acid (pH 5) to give Int. 3E76 (S)-4-(1-(1-(4-(6-((tert-butoxycarbonyl)amino)pyridin-3-yl)-1-methyl-1H-pyrrolo[2,3-b]pyridin-2-yl)ethyl)piperidin-4-yl)benzoic acid (3.1 g).

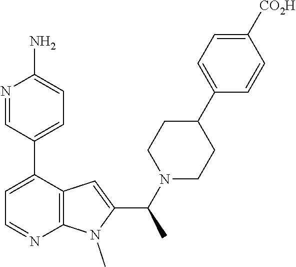 [SMILES: C[C@@H](c1cc2c(-c3ccc(N)nc3)ccnc2n1C)N1CCC(c2ccc(C(=O)O)cc2)CC1]

Int. 3E76 (9.5 g) was stirred ON in dioxane/4M HCl in dioxane (5:4; 90 mL) at 0° C. to RT, concentrated to give Int. 3E83 (S)-4-(1-(1-(4-(6-aminopyridin-3-yl)-1-methyl-1H-pyrrolo[2,3-b]pyridin-2-yl)ethyl)piperidin-4-yl)benzoic acid (8.4 g, HCl salt). Alternatively, Example 1af1l (50 mg) and NaOH (41 mg) were stirred ON at 100° C. in dioxane/water (8:1, 4.5 mL). The mixture was concentrated and triturated in DCM to give a solid. This material was taken up in aq. citric acid to precipitate Int. 3E83 (20 mg).

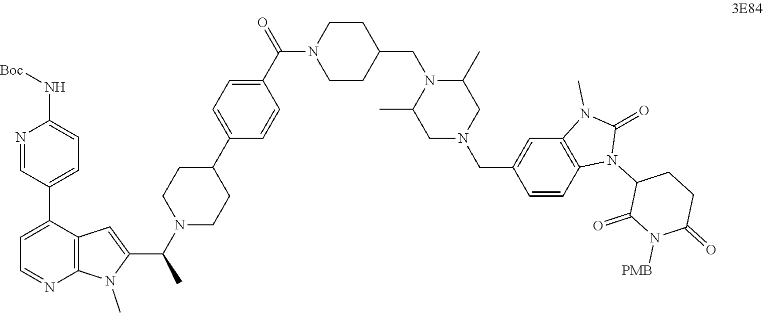

Ints. 3E85/3E76 (0.15 g, HCl salt/0.10 g) and HATU (68 mg) were stirred ON in DMF/DIPEA (24:1, 3.2 mL) and diluted with water to precipitate Int. 3E84 tert-butyl (5-(2-((1S)-1-(4-(4-(4-((4-((1-(1-(4-methoxybenzyl)-2,6-dioxopiperidin-3-yl)-3-methyl-2-oxo-2,3-dihydro-1H-benzo[d]imidazol-5-yl)methyl)-2,6-cis-dimethylpiperazin-1-yl)methyl)piperidine-1-carbonyl)phenyl)piperidin-1-yl)ethyl)-1-methyl-1H-pyrrolo[2,3-b]pyridin-4-yl)pyridin-2-yl)carbamate (0.14 g).

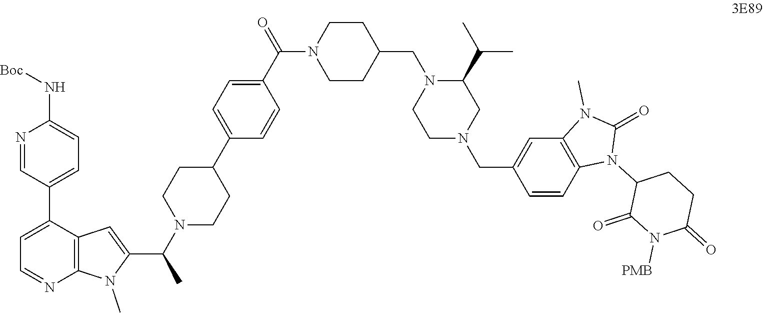

Ints. 3E90/3E76 (0.16 g/0.13 g) and HATU (0.14 g) were stirred ON in DMF/DIPEA (8:1, 2.25 mL, and diluted with water to precipitate Int. 3E89 tert-butyl (5-(2-((1S)-1-(4-(4-(4-(((2S)-2-iso-propyl-4-((1-(1-(4-methoxybenzyl)-2,6-dioxo-piperidin-3-yl)-3-methyl-2-oxo-2,3-dihydro-1H-benzo[d]imidazol-5-yl)methyl)piperazin-1-yl)methyl)-piperidine-1-carbonyl)phenyl)piperidin-1-yl)ethyl)-1-methyl-1H-pyrrolo[2,3-b]pyridin-4-yl)pyridin-2-yl)carbamate (0.19 g).

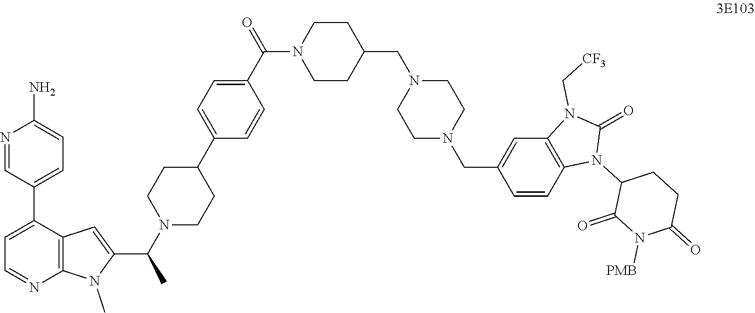

Ints. 3E83/3E104 (84 mg/0.16 g) and HATU (0.11 g) were stirred ON in DMF/DIPEA (26.3:1, 5.2 mL) and diluted with water to precipitate Int. 3E103 3-(5-((4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)piperazin-1-yl)methyl)-2-oxo-3-(2,2,2-trifluoroethyl)-2,3-dihydro-1H-benzo[d]imidazol-1-yl)-1-(4-methoxybenzyl)piperidine-2,6-dione (0.16 g).

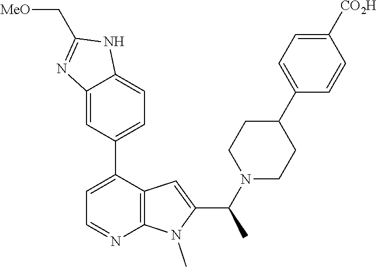 [SMILES: COCc1nc2cc(-c3ccnc4c3cc([C@H](C)N3CCC(c5ccc(C(=O)O)cc5)CC3)n4C)ccc2[nH]1]

A solution of Int. 3E308/3E309 (0.6 g) in DCM/4M HCl in dioxane (1:1; 12 mL) was stirred 2 h at 0° C. to RT and concentrated to give Int. 3E120 (S)-4-(1-(1-(4-(2-(methoxymethyl)-1H-benzo[d]imidazol-5-yl)-1-methyl-1H-(0.4 g, HCl salt).

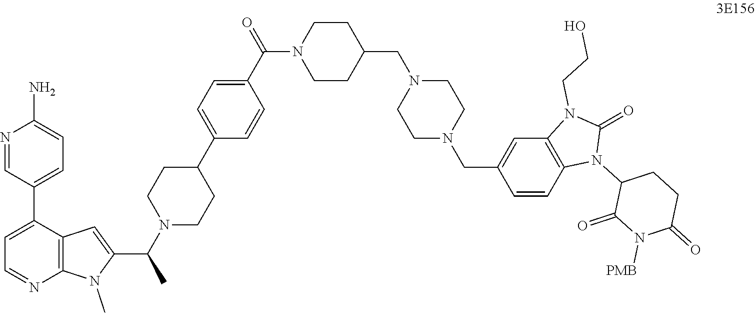

Ints. 3E157/3E83 (0.1 g/54 mg) and HATU (73 mg) were stirred ON in DMF/DIPEA (23:1, 3.1 mL) and diluted with water to precipitate Int. 3E156 3-(5-((4-((1-(4-(1-((S)-1-(4-(6-aminopyridin-3-yl)-1-methyl-1H-pyrrolo[2,3-b]pyridin-2-yl)ethyl)piperidin-4-yl)-benzoyl)piperidin-4-yl)methyl)piperazin-1-yl)methyl)-3-(2-hydroxyethyl)-2-oxo-2,3-dihydro-1H-benzo[d]imidazol-1-yl)-1-(4-methoxybenzyl)piperidine-2,6-dione (90 mg).

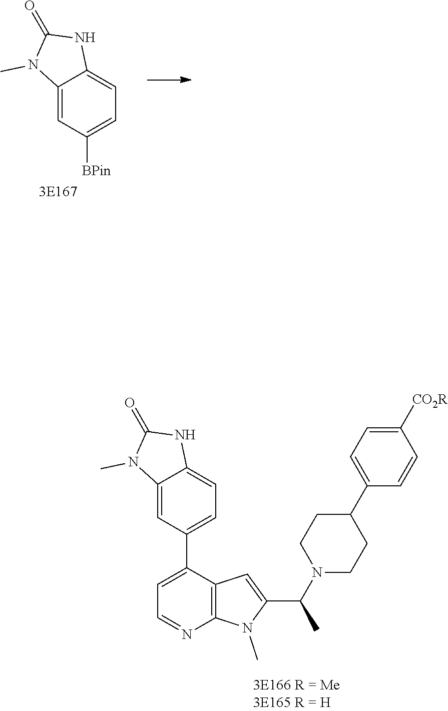

6-Bromo-1-methyl-1,3-dihydro-2H-benzo[d]imidazol-2-one (0.50 g), B₂Pin₂ (0.84 g), PdDPPFCl₂-DCM (0.18 g), and KOAc (0.43 g) were degassed in dioxane (10 mL) and stirred ON at 80° C. The OL (water/EtOAc) was dried, concentrated, and purified by FC (pentane/EtOAc 1:0 to 2:3) to give Int. 3E167 1-methyl-6-(4,4,5,5-tetramethyl-1,3,2-dioxaborolan-2-yl)-1,3-dihydro-2H-benzo[d]imidazol-2-one (0.60 g). Ints. 3E167/3E35 (0.36 g/0.40 g), PdDPPFCl₂-DCM (72 mg), and Na₂CO₃ (0.19 g) were degassed in dioxane/water (10:1; 5.5 mL) and stirred ON at 110° C., diluted with EtOAc, filtered, and concentrated. The residue was purified by FC (DCM/MeOH 1:0 to 73:7) to give Int. 3E166 methyl (S)-4-(1-(1-(1-methyl-4-(3-methyl-2-oxo-2,3-dihydro-1H-benzo[d]imidazol-5-yl)-1H-pyrrolo[2,3-b]pyridin-2-yl)ethyl)-piperidin-4-yl)benzoate (0.32 g). This material (0.3 g) and LiOH—H₂O (0.1 g) were stirred ON in MeOH/THF/water (2:2:1; 15 mL), concentrated, and triturated in dilute aq. HCl to give Int. 3E165 (S)-4-(1-(1-(1-methyl-4-(3-methyl-2-oxo-2,3-dihydro-1H-benzo[d]imidazol-5-yl)-1H-pyrrolo[2,3-b]pyridin-2-yl)ethyl)piperidin-4-yl)benzoic acid (0.22 g).

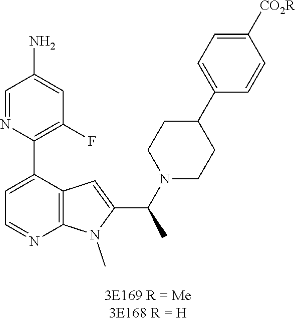

Int. 3E170 (0.5 g), 6-bromo-5-fluoro-pyridin-3-amine (0.19 g), K₃PO₄ (0.42 g), and PdDPPFCl₂-DCM (41 mg) were degassed in dioxane/water (10:1; 5.5 mL) and stirred 1 h at 100° C. under MW conditions. The OL (EtOAc/water) was dried, concentrated, and purified by FC (EtOAc) to give Int. 3E169 (0.3 g). 0.18 g of this material and LiOH—H₂O (45 mg) were stirred ON in MeOH/THF/water (10:5:3; 9 mL) at 0° C. to RT, concentrated, and triturated in dilute aq. citric acid to give Int. 3E168 4-[1-[(1S)-1-[4-(5-amino-3-fluoro-2-pyridyl)-1-methyl-pyrrolo[2,3-b]pyridin-2-yl]ethyl]-4-piperidyl]benzoic acid (0.16 g).

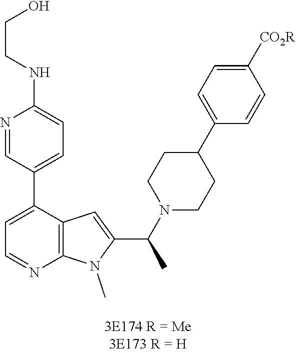

Int. 3E35 (0.30 g), 2-[[5-(4,4,5,5-tetramethyl-1,3,2-dioxaborolan-2-yl)-2-pyridyl]-amino]ethanol (0.26 g), K₃PO₄ (0.28 g), and RuPhos Pd G2 (26 mg) were degassed in dioxane/water (9:1; 10 mL) and stirred ON at 80-85° C., diluted with EtOAc and filtered. The OL was dried, concentrated, and purified by FC (pentane/EtOAc 7:3 to 1:1) to give Int. 3E174 methyl (S)-4-(1-(1-(4-(6-((2-hydroxyethyl)-amino)-pyridin-3-yl)-1-methyl-1H-pyrrolo[2,3-b]-pyridin-2-yl)ethyl)piperidin-4-yl)benzoate (0.35 g). 0.31 g of this material and LiOH—H₂O (74 mg) were stirred ON in MeOH/THF/water (10:5:3; 9 mL), concentrated, and triturated in dilute aq. citric acid to give Int. 3E173 (S)-4-(1-(1-(4-(6-((2-hydroxyethyl)amino)pyridin-3-yl)-1-methyl-1H-pyrrolo[2,3-b]pyridin-2-yl)ethyl)piperidin-4-yl)benzoic acid (0.25 g).

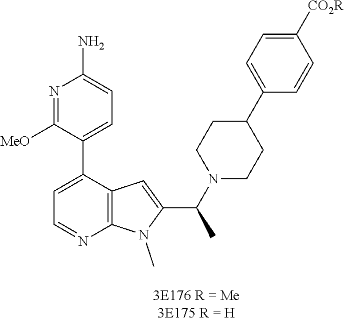

Int. 3E170 (0.50 g), 5-bromo-6-methoxy-pyridin-2-amine (0.24 g), K₃PO₄ (0.50 g), and PdDPPFCl₂-DCM (48 mg) were degassed in dioxane/water (9:1; 10 mL) and stirred 1 h at 100° C. The filtrate after filtration was diluted with EtOAc. The OL was dried, concentrated, and purified by FC (pentane/EtOAc 7:3 to 1:1) to give Int. 3E176 methyl 4-[1-[(1S)-1-[4-(6-amino-2-methoxy-3-pyridyl)-1-methyl-pyrrolo[2,3-b]pyridin-2-yl]ethyl]-4-piperidyl]benzoate (0.48 g). 0.40 g of this material and LiOH—H₂O (98 mg) were stirred ON in MeOH/THF/water (10; 5:3; 9 mL) at 0° C. to RT, concentrated, and triturated in dilute aq. citric acid to give Int. 3E175 (S)-4-(1-(1-(4-(6-amino-2-methoxypyridin-3-yl)-1-methyl-1H-pyrrolo[2,3-b]pyridin-2-yl)ethyl)piperidin-4-yl)benzoic acid (0.20 g).

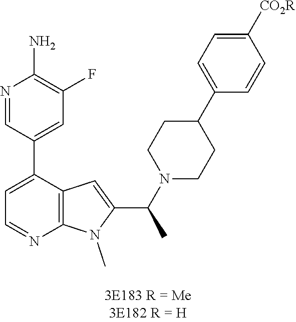

Int. 3E170 (0.30 g), 5-bromo-3-fluoropyridin-2-amine (0.11 mg), K₃PO₄ (0.25 g), and PdDPPFCl₂-DCM (24 mg) were degassed in dioxane/water (10:1; 3.3 mL) and stirred 1 h at 100° C. under MW conditions. The OL (EtOAc/water) was dried, concentrated, and purified by FC (EtOAc) to give Int. 3E183 methyl (S)-4-(1-(1-(4-(6-amino-5-fluoropyridin-3-yl)-1-methyl-1H-pyrrolo[2,3-b]pyridin-2-yl)ethyl)piperidin-4-yl)benzoate (0.25 g). Int. 3E182 (S)-4-(1-(1-(4-(6-amino-5-fluoropyridin-3-yl)-1-methyl-1H-pyrrolo[2,3-b]pyridin-2-yl)ethyl)piperidin-4-yl)benzoic acid (0.20 g) was prepared similarly to 3E179 from Int. 3E183 (0.42 g).

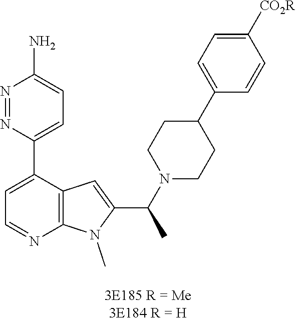

Int. 3E177 (0.44 g), 6-bromopyridazin-3-amine (0.18 g), Na₂CO₃ (0.33 g), and PdDPPFCl₂-DCM (84 mg) were degassed in dioxane/water (4:1; 8 mL) and stirred ON at 110° C., diluted with EtOAc and filtered. The OL was dried, concentrated, and purified by FC (pentane/EtOAc 1:0 to 2:3) to give Int. 3E185 methyl (S)-4-(1-(1-(4-(6-aminopyridazin-3-yl)-1-methyl-1H-pyrrolo[2,3-b]pyridin-2-yl)ethyl)piperidin-4-yl)benzoate (0.20 g). A solution of Int. 3E185 (0.20 g) in dioxane/6M aq. HCl (2:0.8, 2.8 mL) was stirred ON at 80° C. and concentrated to give Int. 3E184 (S)-4-(1-(1-(4-(6-aminopyridazin-3-yl)-1-methyl-1H-pyrrolo[2,3-b]pyridin-2-yl)ethyl)piperidin-4-yl)benzoic acid (0.20 g, HCl salt).

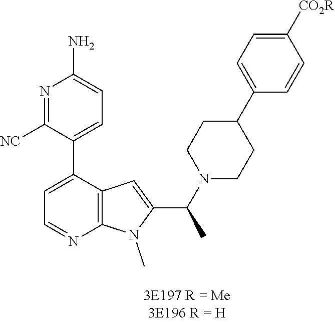

Int. 3E35 (0.25 g), 6-amino-3-(4,4,5,5-tetramethyl-1,3,2-dioxaborolan-2-yl)picolinonitrile (0.19 g), K₃PO₄ (0.23 g), and PdDPPFCl₂-DCM (40 mg) were degassed in dioxane/water (10:1; 3.3 mL) and stirred 2.5 h at 120° C. under MW conditions. The OL (water/EtOAc) was washed with brine, dried, concentrated, and purified by FC (EtOAc/pentane 3:7 to 3:2) to give Int. 3E197 methyl 4-[1-[(1S)-1-[4-(6-amino-2-cyano-3-pyridyl)-1-methyl-pyrrolo-[2,3-b]pyridin-2-yl]ethyl]-4-piperidyl]benzoate (0.2 g). Int. 3E197 (0.25 g) and LiOH—H₂O (64 mg) were stirred ON in MeOH/THF/water (5:5:2; 12 mL), concentrated, and triturated in dilute aq. citric acid to give Int. 3E196 (S)-4-(1-(1-(4-(6-amino-2-cyanopyridin-3-yl)-1-methyl-1H-pyrrolo[2,3-b]pyridin-2-yl)ethyl)piperidin-4-yl)benzoic acid (0.20 g).

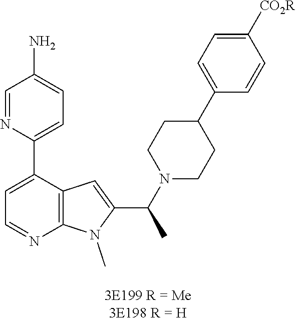

Int. 3E170 (0.50 g), 6-bromopyridin-3-amine (0.17 g), K₃PO₄ (0.42 g), and PdDPPFCl₂-DCM (77 mg) were degassed in dioxane/water (10:1; 5.5 mL) and stirred 2 h at 130° C. under MW conditions and filtered. The OL (water/EtOAc) was dried, concentrated, and purified by FC (pentane/EtOAc 7:3 to 1:1) to give Int. 3E199 methyl 4-[1-[(1S)-1-[4-(5-amino-2-pyridyl)-1-methyl-pyrrolo[2,3-b]pyridin-2-yl]ethyl]-4-piperidyl]benzoate (0.15 g). A mixture of Int. 3E199 (0.20 g) and LiOH—H₂O (87 mg) in MeOH/THF/water (10:5:2.5; 17.5 mL) were stirred ON, concentrated, and triturated in dilute aq. citric acid to precipitate Int. 3E198 (S)-4-(1-(1-(4-(5-aminopyridin-2-yl)-1-methyl-1H-pyrrolo[2,3-b]pyridin-2-yl)ethyl)piperidin-4-yl)benzoic acid (0.15 g).

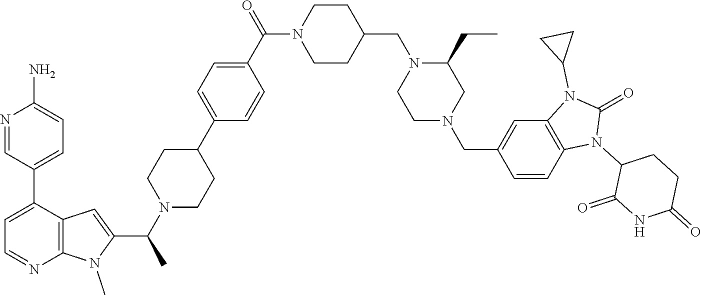

Ints. 3E209/3E68 (0.55 g/0.50 g) and HATU (0.13 g) were stirred ON in DMF/DIPEA (7.1:1, 5.7 mL) and diluted with water to precipitate Int. 3E208 3-(5-(((S)-4-((1-(4-(1-((S)-1-(4-(6-aminopyridin-3-yl)-1-methyl-1H-pyrrolo[2,3-b]pyridin-2-yl)ethyl)piperidin-4-yl)benzoyl)piperidin-4-yl)methyl)-3-ethylpiperazin-1-yl)methyl)-3-cyclopropyl-2-oxo-2,3-dihydro-1H-benzo[d]imidazol-1-yl)piperidine-2,6-dione (0.22 mg, after HPLC-purification).

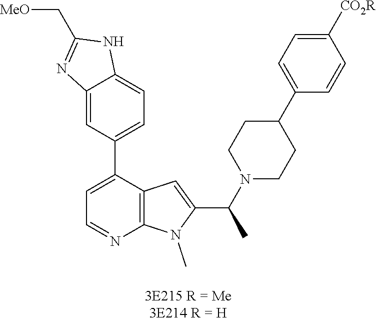

3E310/3E311 (0.77 g) was stirred ON in DCM/TFA (1.3:1, 19.3 mL), concentrated, and purified by FC (DCM/MeOH 1:0 to 3:2) to give Int. 3E215 methyl (S)-4-(1-(1-(4-(2-(methoxymethyl)-1H-benzo[d]-imidazol-5-yl)-1-methyl-1H-pyrrolo[2,3-b]pyridin-2-yl)ethyl)piperidin-4-yl)benzoate (0.60 g). This material was stirred in THF/MeOH/2M aq. NaOH (2:1:1; 12 mL) and HPLC-purified to give Int. 3E214 (S)-4-(1-(1-(4-(2-(methoxymethyl)-1H-benzo[d]imidazol-5-yl)-1-methyl-1H-pyrrolo[2,3-b]pyridin-2-yl)ethyl)piperidin-4-yl)benzoic acid (0.58 g).

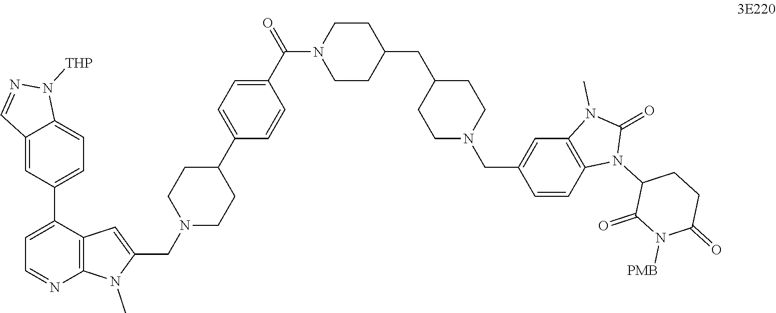

Int. 3A2 (20 mg) and HATU (18 mg) were stirred 0.3 h in DMF/DIPEA (67:1, 2 mL). Int. 3E221 (26 mg) was added and stirring continued ON. The OL (water/2MeTHF) was washed with sat. aq. NaHCO₃ and brine, dried, concentrated, and purified by FC (DCM/MeOH 1:0 to 9:1) to give Int. 3E220 1-(4-methoxybenzyl)-3-(3-methyl-5-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-piperidin-1-yl)methyl)-2-oxo-2,3-dihydro-1H-benzo[d]imidazol-1-yl)piperidine-2,6-dione (18 mg).

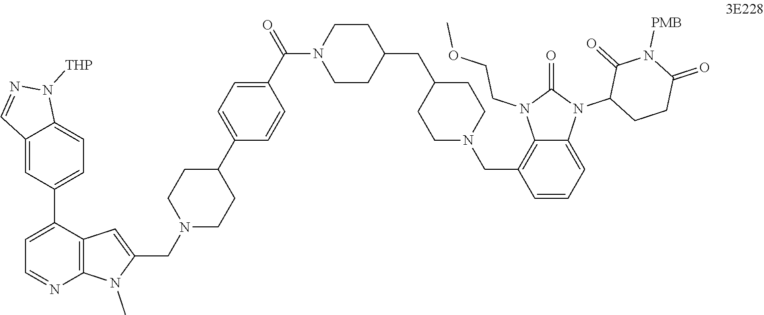

Ints. 3A2/3E229 (34 mg/30 mg) and HATU (24 mg) were stirred 2 h in DMF/DIPEA (24:1, 2.08 mL), filtered, and HPLC-purified to give Int. 3E228 1-(4-methoxybenzyl)-3-(3-(2-methoxyethyl)-4-((4-((1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)methyl)-piperidin-1-yl)methyl)-2-oxo-2,3-dihydro-1H-benzo[d]imidazol-1-yl)piperidine-2,6-dione (47 mg).

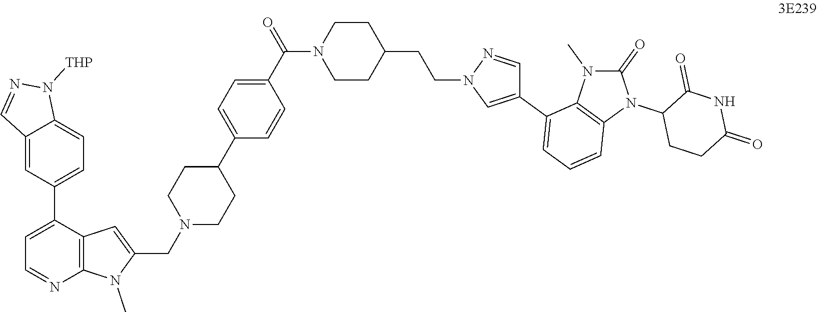

Int. 3E240 (17 mg, HCl salt), Int. 3A2 (20 mg) and HATU (14 mg) were stirred ON in ACN/DIPEA/DMF (10:1:1, 0.7 mL). The OL (aq. NaHCO₃/DCM) was washed with brine, dried, concentrated, and purified by FC (DCM/MeOH 97:3) to give Int. 3E239 3-[3-methyl-4-[1-[2-[1-[4-[1-[[1-methyl-4-(1-tetrahydropyran-2-ylindazol-5-yl)pyrrolo[2,3-b]pyridin-2-yl]methyl]-4-piperidyl]benzoyl]-4-piperidyl]ethyl]pyrazol-4-yl]-2-oxo-benzimidazol-1-yl]piperidine-2,6-dione (13 mg).

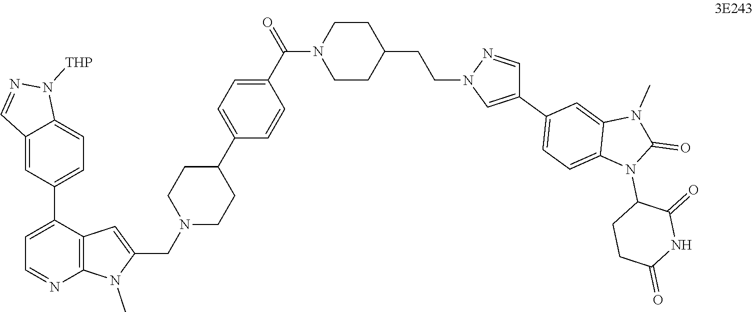

Int. 3E244 (26 mg, HCl salt), Int. 3A2 (31 mg) and HATU (22 mg) were stirred 1 h in DMF/DIPEA (2.5:1, 0.28 mL). The OL (aq. NaHCO₃/DCM) was washed with brine, dried, concentrated, and purified by FC (DCM/MeOH 97:3 to 96:4) to give Int. 3E243 3-(3-methyl-5-(1-(2-(1-(4-(1-((1-methyl-4-(1-(tetrahydro-2H-pyran-2-yl)-1H-indazol-5-yl)-1H-pyrrolo[2,3-b]pyridin-2-yl)methyl)piperidin-4-yl)benzoyl)piperidin-4-yl)ethyl)-1H-pyrazol-4-yl)-2-oxo-2,3-dihydro-1H-benzo[d]imidazol-1-yl)piperidine-2,6-dione (29 mg).

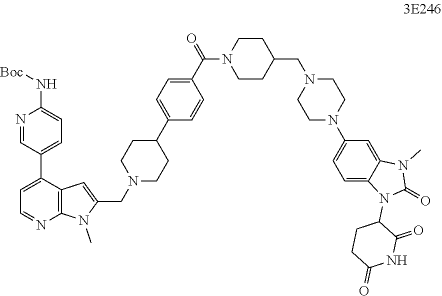 [SMILES: Cn1c(CN2CCC(c3ccc(C(=O)N4CCC(CN5CCN(c6ccc7c(c6)n(C)c(=O)n7C6CCC(=O)NC6=O)CC5)CC4)cc3)CC2)cc2c(-c3ccc(NC(=O)OC(C)(C)C)nc3)ccnc21]

Ints. 3E56/3E247 (35 mg/47 mg) and HATU (28 mg) were stirred ON in DMF/DIPEA (6.3:1, 1.2 mL). The OL (water/DCM) was washed with brine, dried, concentrated, and purified by FC (DCM/Et₃N/MeOH 95:5:0 to 85:5:10) to give Int. 3E246 tert-butyl (5-(2-((4-(4-(4-((4-(1-(2,6-dioxopiperidin-3-yl)-3-methyl-2-oxo-2,3-dihydro-1H-benzo[d]-imidazol-5-yl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (53 mg).

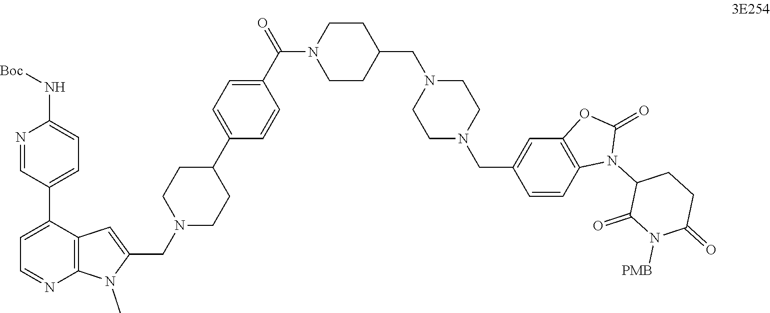

Ints. 3E56/3E255 (43 mg/40 mg) and HATU (31 mg) were stirred ON in DMF/DIPEA (8.3:1, 1.1 mL. The OL (water/EtOAc) was dried, concentrated, and purified by FC (DCM/MeOH 1:0 to 95:5) to give Int. 3E254 tert-butyl (5-(2-((4-(4-(4-((4-((3-(1-(4-methoxybenzyl)-2,6-dioxopiperidin-3-yl)-2-oxo-2,3-dihydrobenzo[d]oxazol-6-yl)-methyl)piperazin-1-yl)methyl)piperidine-1-carbonyl)phenyl)piperidin-1-yl)methyl)-1-methyl-1H-pyrrolo[2,3-b]pyridin-4-yl)pyridin-2-yl)carbamate (48 mg).

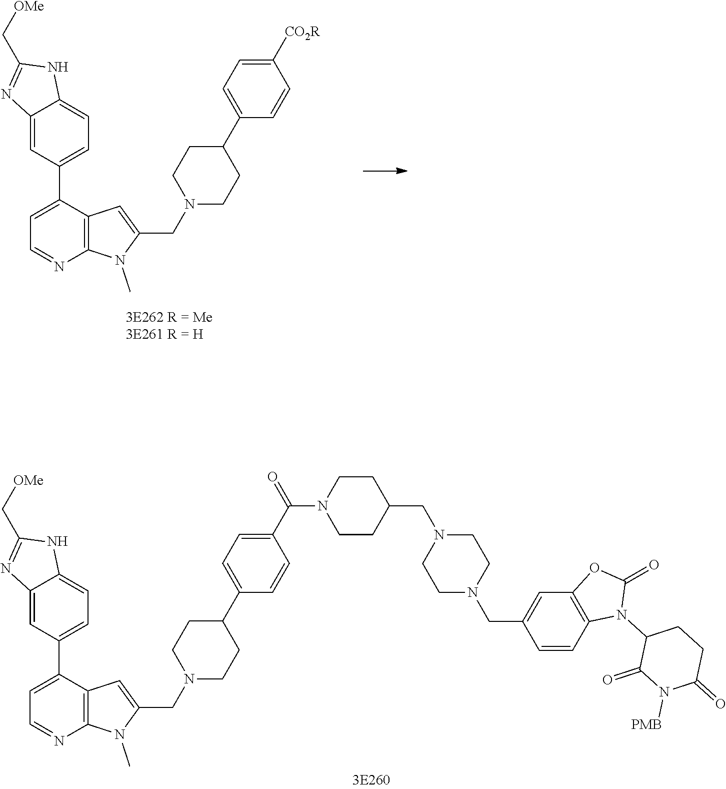

Ints. 1AC5/3E8 (1.2 g/1.1 g), PdDPPFCl₂-DCM (0.33 g), and NaHCO₃ (1.1 g) were degassed in dioxane/water (10:1; 22 mL) and stirred ON at 90° C., filtered, concentrated, and purified by FC (DCM/MeOH 9:1) and by HPLC to give Int. 3E262 methyl 4-(1-((4-(2-(methoxy-methyl)-1H-benzo[d]imidazol-5-yl)-1-methyl-1H-pyrrolo-[2,3-b]pyridin-2-yl)methyl)piperidin-4-yl)benzoate (0.58 g). A solution of Int. 3E262 (0.12 g) in THF/MeOH/2M aq. NaOH (2:1:0.04, 5 mL) was stirred ON and then acidified with aq. HCl to precipitate Int. 3E261 4-[1-[[4-[2-(methoxymethyl)-1H-benzimidazol-5-yl]-1-methyl-pyrrolo[2,3-b]pyridin-2-yl]methyl]-4-piperidyl]benzoic acid (0.66 g). Ints. 3E255/3E261 (40 mg/42 mg) and HATU (31 mg) were stirred ON in DMF/DIPEA (1:0.06, 2.1 mL). The OL (water/EtOAc) was concentrated and purified by FC (DCM/MeOH 1:0 to 3:1) to give Int. 3E260 (48 mg).

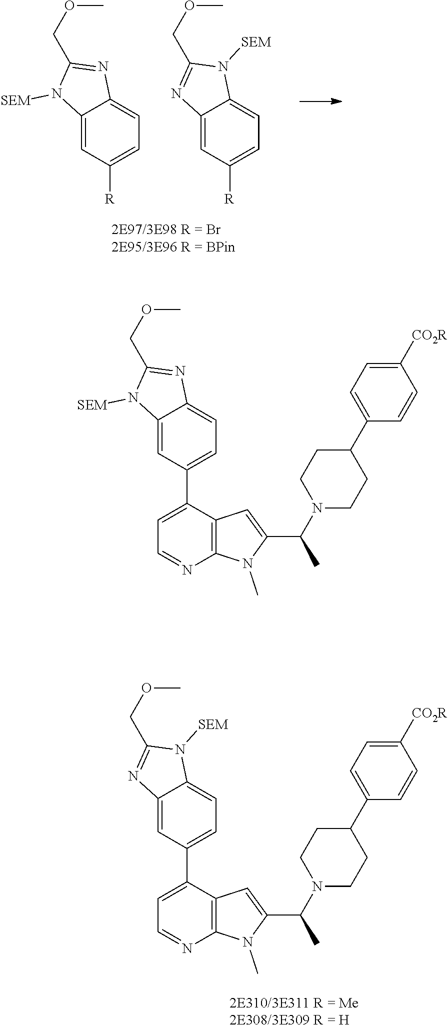

5-Bromo-2-(methoxymethyl)-1H-benzo[d]imidazole (25 g) was dissolved in DMF (150 mL). NaH (6.2 g) was added at 0° C. The mixture was stirred 0.25 h at 0° C. SEM-Cl (20.8 g) was added and stirring continued 0.25 h at 0° C. The OL (water/EtOAc) was dried and concentrated. The residue was combined with another batch prepared similarly and purified by FC (pentane/EtOAc 3:7) to give Int. 3E97/3E98 5-bromo-2-(methoxymethyl)-1-((2-(trimethylsilyl)-ethoxy)methyl)-1H-benzo[d]imidazole and 6-bromo-2-(methoxymethyl)-1-((2-(trimethyl-silyl)ethoxy)methyl)-1H-benzo[d]imidazole (30 g). 20 g of Int. 3E97/3E98, B₂Pin₂ (15 g), KOAc (15.8 g), and PdDPPFCl₂-DCM (2.2 g) were degassed in dioxane (100 mL) and stirred ON at 90° C., filtered, and concentrated. The residue was combined with another batch prepared in similarly on 10 g scale and purified by FC (pentane/EtOAc 1:4) to give Int. 3E95/3E96 2-(methoxymethyl)-6-(4,4,5,5-tetramethyl-1,3,2-dioxaborolan-2-yl)-1-((2-(trimethylsilyl)ethoxy)methyl)-1H-benzo[d]imidazole and 2-(methoxymethyl)-5-(4,4,5,5-tetramethyl-1,3,2-dioxaborolan-2-yl)-1-((2-(trimethylsilyl)ethoxy)methyl)-1H-benzo[d]imidazole (20 g). Ints. 3E35 (2.0 g) and 3E95/3E96 (2.0 g), NaHCO₃ (0.74 g), and PdDPPFCl₂-DCM (0.36 g) were degassed in dioxane/water (4.5:1, 22 mL) and stirred ON at 80° C. The OL (water/EtOAc) was washed with brine, dried, concentrated, and purified by FC (hexane/EtOAc 4:1 to 7:3) to give Int. 3E310/3E311 methyl (S)-4-(1-(1-(4-(2-(methoxymethyl)-1-((2-(trimethyl-silyl)ethoxy)methyl)-1H-benzo[d]imidazol-5-yl)-1-methyl-1H-pyrrolo[2,3-b]pyridin-2-yl)ethyl)piperidin-4-yl)benzoate and methyl (S)-4-(1-(1-(4-(2-(methoxymethyl)-1-((2-(trimethylsilyl)-ethoxy)methyl)-1H-benzo[d]-imidazol-6-yl)-1-methyl-1H-pyrrolo[2,3-b]pyridin-2-yl)ethyl)piperidin-4-yl)benzoate (2.7 g). This material and LiOH—H₂O (0.51 g) were stirred ON in THF/MeOH/water (2:2:1; 50 mL), concentrated, and triturated in dilute aq. citric acid to precipitate Int. 3E308/3E309 (S)-4-(1-(1-(4-(2-(methoxy-methyl)-1-((2-(trimethylsilyl)ethoxy)-methyl)-1H-benzo[d]imidazol-5-yl)-1-methyl-1H-pyrrolo[2,3-b]pyridin-2-yl)ethyl)piperidin-4-yl)benzoic acid and (S)-4-(1-(1-(4-(2-(methoxymethyl)-1-((2-(trimethylsilyl)ethoxy)methyl)-1H-benzo[d]imidazol-6-yl)-1-methyl-1H-pyrrolo[2,3-b]pyridin-2-yl)ethyl)piperidin-4-yl)benzoic acid (1.56 g).

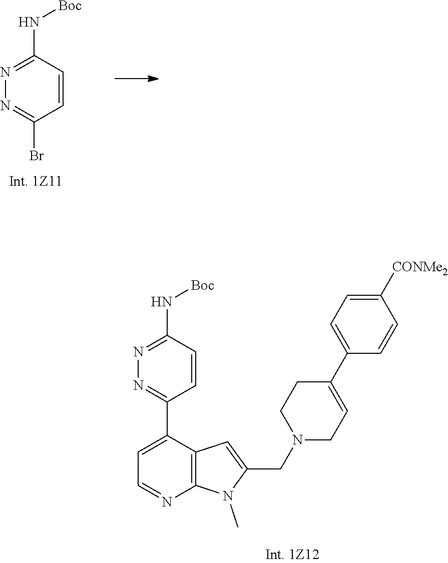

Ints. 1Z11/1Z10 (0.43 g/0.72 g), K₂CO₃ (0.60 g), and [2-(di-1-adamantyl-phosphino)-2′,4′,6′-triisopropyl-3,6-dimethoxybiphenyl][2-(2′-amino-1,1′-biphenyl)]palladium(II) methanesulfonate (29 mg) were stirred 48 h in dioxane (20 mL) at 90° C. under an inert atmosphere. The OL (water/MTBE) was concentrated to give Int. 1Z12 tert-butyl (6-(2-((4-(4-(dimethylcarbamoyl)phenyl)-3,6-dihydropyridin-1(2H)-yl)methyl)-1-methyl-1H-pyrrolo[2,3-b]pyridin-4-yl)pyridazin-3-yl)carbamate (0.75 g).

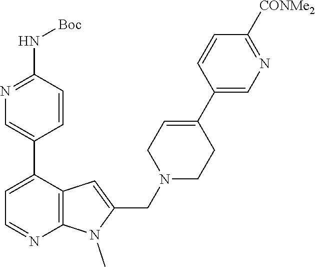

PdDPPFCl₂-DCM (16 mg), Int. 1Z50 (0.4 g), tert-butyl (5-(4,4,5,5-tetramethyl-1,3,2-dioxaborolan-2-yl)pyridin-2-yl)carbamate (0.3 g), and K₂CO₃ (0.4 g) were stirred ON in dioxane/H₂O (5:1, 6 mL) at 100° C. under an inert atmosphere. The mixture was concentrated to give Int. 1Z13 tert-butyl (5-(2-((6-(dimethylcarbamoyl)-3′,6′-dihydro-[3,4′-bipyridin]-1′(2′H)-yl)methyl)-1-methyl-1H-pyrrolo[2,3-b]pyridin-4-yl)pyridin-2-yl)carbamate (0.35 g).

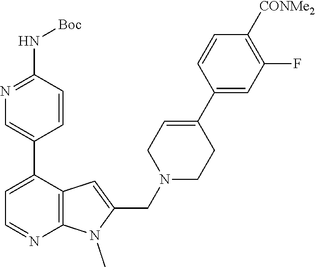

PdDPPFCl₂-DCM (4 mg), Int. 1Z54 (0.25 g), tert-butyl (5-(4,4,5,5-tetramethyl-1,3,2-dioxaborolan-2-yl)pyridin-2-yl)carbamate (0.19 g), and K₂CO₃ (0.22 g) were stirred ON in dioxane/H₂O (5:1, 6 mL) at 100° C. under an inert atmosphere. The mixture was filtered and concentrated to give Int. 1Z21 tert-butyl (5-(2-((4-(4-(dimethylcarbamoyl)-3-fluorophenyl)-3,6-dihydropyridin-1(2H)-yl)methyl)-1-methyl-1H-pyrrolo[2,3-b]pyridin-4-yl)pyridin-2-yl)carbamate (0.27 g).

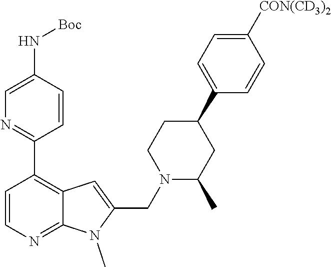

Ints. 1Q4/1D17 (0.20 g/0.14 g) and KI (9 mg) were stirred 16 h in DMF/DIPEA (4:1, 2.5 mL). The OL (water/EtOAc) was dried, filtered, concentrated, and purified by FC (DCM/MeOH 19:1) to give Int. 1Z22 tert-butyl (6-(2-(((2R,4R)-4-(4-(bis(methyl-d₃)carbamoyl)phenyl)-2-methylpiperidin-1-yl)methyl)-1-methyl-1H-pyrrolo[2,3-b]pyridin-4-yl)pyridin-3-yl)carbamate (0.12 g).

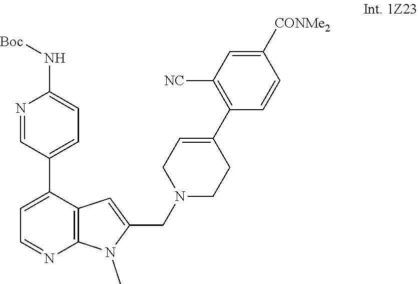

PdDPPFCl₂-DCM (1 mg), tert-butyl (5-(4,4,5,5-tetra-methyl-1,3,2-dioxaborolan-2-yl)pyridin-2-yl)carbamate (44 mg), Int. 1Z27 (60 mg), and K₂CO₃ (52 mg) were stirred ON in dioxane/H₂O (5:1, 6 mL) at 100° C. under an inert atmosphere. The mixture was concentrated to give Int. 1Z23 tert-butyl (5-(2-((4-(2-cyano-4-(dimethylcarbamoyl)-phenyl)-3,6-dihydropyridin-1(2H)-yl)methyl)-1-methyl-1H-pyrrolo[2,3-b]pyridin-4-yl)pyridin-2-yl)carbamate (60 mg).

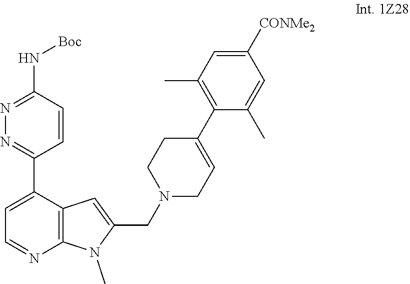

PdDPPFCl₂-DCM (50 mg), Ints. 1Z11/1Z30 (0.2 g/0.3 g), and K₂CO₃ (0.5 g) were stirred 12 h in dioxane (5:1, 18 mL) at 80° C. under an inert atmosphere. The OL (water/EtOAc) was concentrated to give Int. 1Z28 tert-butyl (6-(2-((4-(4-(dimethylcarbamoyl)-2,6-dimethylphenyl)-3,6-dihydropyridin-1(2H)-yl)methyl)-1-methyl-1H-pyrrolo[2,3-b]pyridin-4-yl)pyridazin-3-yl)carbamate (0.3 g).

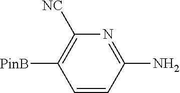

6-Amino-3-bromopicolinonitrile (10 g), bis(pinacolato)diboron (26 g), PdDPPFCl₂-DCM (4.1 g), and KOAc (15 g) were stirred 0.5 h in THF (200 mL) at 90° C. The mixture was filtered through celite. The OL (water/EtOAc) was dried, filtered, concentrated, and washed with Et₂O. The residue was purified by FC (hexane/EtOAc 5:4), concentrated, and washed with hexane to give Int. 1Z32 6-amino-3-(4,4,5,5-tetramethyl-1,3,2-dioxaborolan-2-yl)picolinonitrile (5.2 g).

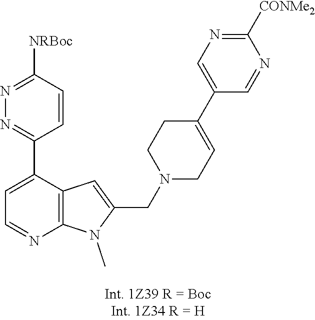

Int. 1Z38 (90 mg), 6-bromo-N,N-dimethylpyridazin-3-amine (40 mg), K₂CO₃ (75 mg), and PdDPPFCl₂-DCM (1 mg) were stirred ON in dioxane/H₂O (20:1, 21 mL) at 100° C. under an inert atmosphere. The mixture was filtrated, and concentrated to give Int. 1Z39 5-(1-((4-(6-(dimethylamino)pyridazin-3-yl)-1-methyl-1H-pyrrolo[2,3-b]pyridin-2-yl)methyl)-1,2,3,6-tetrahydropyridin-4-yl)-N,N-dimethylpyrimidine-2-carboxamide (75 mg). Ints. 1Z36/1Z37 (0.72 g/1.2 g), K₂CO₃ (0.99 g), and [2-(di-1-adamantylphosphino)-2′,4′,6′-triisopropyl-3,6-dimethoxybiphenyl][2-(2′-amino-1,1′-biphenyl)]palladium(II) methanesulfonate (48 mg) were stirred 48 h in dioxane (20 mL) at 90° C. The OL (water/MTBE) was concentrated to give Int. 1Z34 tert-butyl (6-(2-((4-(4-(dimethylcarbamoyl)phenyl)piperidin-1-yl)methyl)-1-methyl-1H-pyrrolo[2,3-b]pyridin-4-yl)pyridazin-3-yl)carbamate (0.5 g).

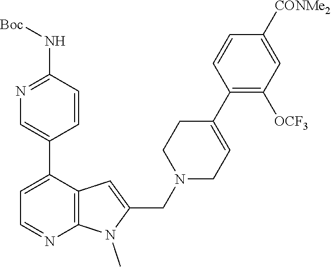

PdDPPFCl₂-DCM (1 mg), Int. 1Z136 (60 mg), tert-butyl 4-(4,4,5,5-tetramethyl-1,3,2-dioxaborolan-2-yl)-3,6-dihydropyridine-1(2H)-carboxylate (39 mg), and K₂CO₃ (46 mg) were stirred ON in dioxane/H₂O (5:1, 6 mL) at 100° C. under an inert atmosphere. The mixture was concentrated to give Int. 1Z49 tert-butyl (5-(2-((4-(4-(dimethylcarbamoyl)-2-(trifluoromethoxy)phenyl)-3,6-dihydropyridin-1(2H)-yl)methyl)-1-methyl-1H-pyrrolo[2,3-b]pyridin-4-yl)pyridin-2-yl)carbamate (60 mg).

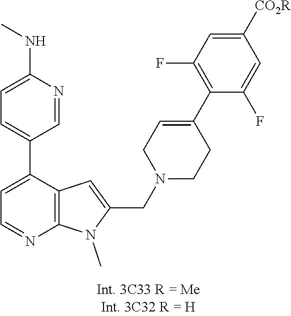

PdDPPFCl₂-DCM (0.17 g), Int. 3C31 (1 g), N-methyl-5-(4,4,5,5-tetramethyl-1,3,2-dioxaborolan-2-yl)-pyridin-2-amine (0.59 g), and NaHCO₃ (0.53 g) were stirred 3 h in dioxane/H₂O (5:1, 24 mL) at 75° C. under an inert atmosphere. The OL (water/EtOAc) was dried, filtered, concentrated, and purified by FC (PE/EtOAc 2:1 to 3:2) to give Int. 3C33 methyl 3,5-difluoro-4-(1-((1-methyl-4-(6-(methylamino)-pyridin-3-yl)-1H-pyrrolo[2,3-b]pyridin-2-yl)methyl)-1,2,3,6-tetrahydropyridin-4-yl)benzoate (0.80 g). LiOH—H₂O (0.13 g) and Int. 3C33 (0.80 g) were stirred 12 h in THF/H₂O (5:1, 22 mL). The residue after concentration was diluted with aq. citric acid to precipitate Int. 3C32 3,5-difluoro-4-(1-((1-methyl-4-(6-(methylamino)-pyridin-3-yl)-1H-pyrrolo[2,3-b]pyridin-2-yl)methyl)-1,2,3,6-tetrahydropyridin-4-yl)benzoic acid (0.60 g).

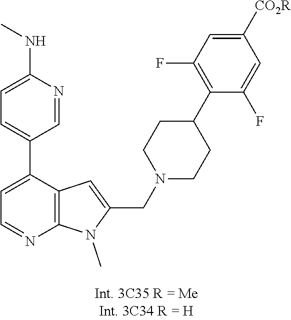

PdDPPFCl₂-DCM (0.17 g), Int. 3C15 (1.1 g), N-methyl-5-(4,4,5,5-tetramethyl-1,3,2-dioxaborolan-2-yl)pyridin-2-amine (0.70 g), and NaHCO₃ (0.58 g) were stirred 16 h in dioxane/H₂O (3:1, 20 mL) at 90° C. under an inert atmosphere. The mixture was filtered. The OL (water/EtOAc) was dried, filtered, concentrated, and purified by FC (PE/EtOAc 2:1 to 3:2) to give Int. 3C35 methyl 3,5-difluoro-4-(1-((1-methyl-4-(6-(methylamino)pyridin-3-yl)-1H-pyrrolo[2,3-b]pyridin-2-yl)methyl)piperidin-4-yl)benzoate (0.90 g). LiOH—H₂O (0.49 g) and Int. 3C35 (1.2 g) were stirred 4 h in THF/MeOH/H₂O (10:3:2, 15 mL) at 0° C. to RT. The residue after concentration was diluted with aq. citric acid to precipitate Int. 3C34 3,5-difluoro-4-(1-((1-methyl-4-(6-(methylamino)pyridin-3-yl)-1H-pyrrolo[2,3-b]pyridin-2-yl)methyl)piperidin-4-yl)benzoic acid (1.0 g).

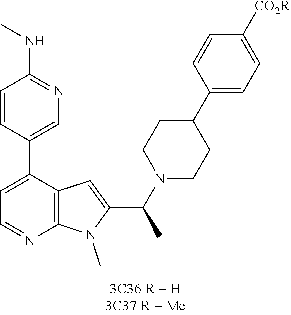

Int. 3E35 (3. 0 g), N-methyl-5-(4,4,5,5-tetramethyl-1,3,2-dioxaborolan-2-yl)pyridin-2-amine (1.8 g), NaHCO₃ (1.1 g) and PdDPPFCl₂-DCM (0.27 g) were stirred 16 h in dioxane/H₂O (4:1, 30 mL) at 85° C. under an inert atmosphere. The OL (water/EtOAc) was dried, filtered, concentrated, and purified by FC (PE/EtOAc 1:1 to 2:3) to give Int. 3C37 methyl (S)-4-(1-(1-(1-methyl-4-(6-(methylamino)-pyridin-3-yl)-1H-pyrrolo[2,3-b]pyridin-2-yl)ethyl)piperidin-4-yl)benzoate (3.0 g). LiOH—H₂O (0.89 g) and Int. 3C37 (2.1 g) were stirred 16 h in THF/MeOH/H₂O (2:2:1, 10 mL) at 0° C. The residue after concentration was stirred in aq. citric acid to precipitate Int. 3C36 (S)-4-(1-(1-(1-methyl-4-(6-(methylamino)pyridin-3-yl)-1H-pyrrolo[2,3-b]pyridin-2-yl)ethyl)piperidin-4-yl)benzoic acid (1.9 g).

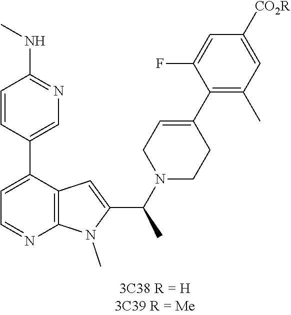

PdDPPFCl₂-DCM (84 mg), Int. 1AF86 (1.0 g), N-methyl-5-(4,4,5,5-tetra-methyl-1,3,2-dioxaborolan-2-yl)-pyridin-2-amine (0.63 g), and NaHCO₃ (0.60 g) were stirred 16 h in dioxane/H₂O (4:1, 10 mL) at 80° C. under an inert atmosphere. The mixture was filtered. The OL (water/EtOAc) was dried, filtered, and concentrated to give Int. 3C39 methyl (S)-3-fluoro-5-methyl-4-(1-(1-(1-methyl-4-(6-(methyl-amino)-pyridin-3-yl)-1H-pyrrolo[2,3-b]pyridin-2-yl)ethyl)-1,2,3,6-tetrahydro-pyridin-4-yl)-benzoate (1.2 g). LiOH—H₂O (0.38 g) and Int. 3C39 (0.95 g) were stirred 6 h in THF/MeOH/H₂O (2:2:1, 25 mL) at 0° C. to 60° C. The residue after concentration was stirred in aq. HCl to precipitate Int. 3C38 (S)-3-fluoro-5-methyl-4-(1-(1-(1-methyl-4-(6-(methylamino)pyridin-3-yl)-1H-pyrrolo[2,3-b]pyridin-2-yl)ethyl)-1,2,3,6-tetrahydro-pyridin-4-yl)benzoic acid (0.80 g).

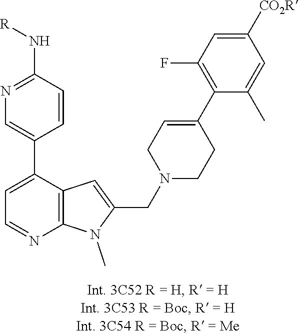

Int. 1AF86 (1.0 g), tert-butyl (5-(4,4,5,5-tetramethyl-1,3,2-dioxaborolan-2-yl)pyridin-2-yl)carbamate (0.72 g), K₃PO₄ (1.7 g), and PdDPPFCl₂-DCM (0.17 g) were stirred 16 h in dioxane/H₂O (5:1, 12 mL) at 90° C. under an inert atmosphere. The mixture was filtered. The OL (water/EtOAc) was dried, filtered, concentrated, and purified by FC (hexane/EtOAc 1:1) to give Int. 3C54 methyl (S)-4-(1-(1-(4-(6-((tert-butoxycarbonyl)amino)pyridin-3-yl)-1-methyl-1H-pyrrolo[2,3-b]pyridin-2-yl)ethyl)-1,2,3,6-tetrahydro-pyridin-4-yl)-3-fluoro-5-methylbenzoate (0.90 g). LiOH (0.32 g) was added to a solution of Int. 3C54 (0.90 g) in THF/H₂O (3:2, 9.5 mL) at 0° C. and stirred 16 h at 60° C. The residue after concentration was stirred in aq. citric acid to precipitate Int. 3C53 (S)-4-(1-(1-(4-(6-((tert-butoxycarbonyl)amino)-pyridin-3-yl)-1-methyl-1H-pyrrolo[2,3-b]pyridin-2-yl)ethyl)-1,2,3,6-tetrahydropyridin-4-yl)-3-fluoro-5-methylbenzoic acid (0.85 g). Int. 3C53 (0.90 g) was stirred 16 h in DCM/4M HCl in dioxane (2:1, 17 mL), neutralized, and concentrated to give Int. 3C52 (S)-4-(1-(1-(4-(6-aminopyridin-3-yl)-1-methyl-1H-pyrrolo[2,3-b]pyridin-2-yl)ethyl)-1,2,3,6-tetrahydropyridin-4-yl)-3-fluoro-5-methylbenzoic acid (0.25 g)

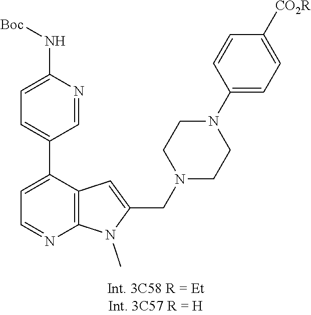

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl)carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)-benzoate (2 g). 6M HCl (20 mL) was added drop wise at 0° C. to a solution of Int. 3C58 (2.1 g) in dioxane (15 mL) and stirred for 6 h at 85° C. The mixture was concentrated and triturated in Et₂O to give Int. 3C57 4-(4-((4-(6-aminopyridin-3-yl)-1-methyl-1H-pyrrolo[2,3-b]pyridin-2-yl)methyl)piperazin-1-yl)benzoic acid (1.5 g).

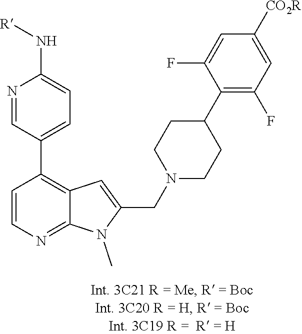

PdDPPFCl₂-DCM (0.15 g), NaHCO₃ (0.47 g), Int. 3C15 (0.90 g), tert-butyl (5-(4,4,5,5-tetramethyl-1,3,2-dioxaborolan-2-yl)pyridin-2-yl)carbamate (0.78 g) were stirred 16 h in dioxane/H₂O (5:1, 24 mL) at 90° C. under an inert atmosphere. The mixture was filtered. The OL (water/EtOAc) was dried, filtered, concentrated, and purified by FC (PE/EtOAc 1:0 to 2:1) to give Int. 3C21 methyl 4-(1-((4-(6-((tert-butoxycarbonyl)amino)pyridin-3-yl)-1-methyl-1H-pyrrolo[2,3-b]pyridin-2-yl)methyl)piperidin-4-yl)-3,5-difluorobenzoate (0.75 g). LiOH—H₂O (0.21 g) and Int. 3C21 (0.75 g) were stirred 16 h in THF/H₂O (5:1, 24 mL) at 60° C. The mixture was diluted with aq. citric acid to precipitate Int. 3C20 4-(1-((4-(6-((tert-butoxycarbonyl)amino)pyridin-3-yl)-1-methyl-1H-pyrrolo[2,3-b]pyridin-2-yl)methyl)piperidin-4-yl)-3,5-difluorobenzoic acid (0.67 g). Int. 3C20 (0.65 g) was stirred 6 h in DCM/4M HCl in dioxane (3:2, 17 mL) at 0° C. to RT, concentrated and washed with Et₂O to give Int. 3C19 4-(1-((4-(6-aminopyridin-3-yl)-1-methyl-1H-pyrrolo[2,3-b]pyridin-2-yl)methyl)piperidin-4-yl)-3,5-difluorobenzoic acid (0.50 g, HCl salt).

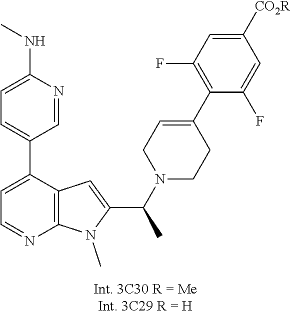

PdDPPFCl₂-DCM (83 mg), Int. 3C28 (0.50 g), N-methyl-5-(4,4,5,5-tetramethyl-1,3,2-dioxaborolan-2-yl)pyridin-2-amine (0.31 g), and NaHCO₃ (0.26 g) were stirred 16 h in dioxane/H₂O (5:1, 6 mL) at 80° C. under an inert atmosphere. The OL (water/EtOAc) was dried, filtered, concentrated, and purified by FC (PE/EtOAc 2:1 to 3:2) to give Int. 3C30 methyl (S)-3,5-difluoro-4-(1-(1-(1-methyl-4-(6-(methyl-amino)pyridin-3-yl)-1H-pyrrolo[2,3-b]pyridin-2-yl)ethyl)-1,2,3,6-tetrahydropyridin-4-yl)-benzoate (0.30 g). LiOH—H₂O (73 mg) and Int. 3C30 (0.30 g) were stirred 6 h in THF/H₂O (3:1, 4 mL) at 60° C. The residue after concentration was diluted with aq. citric acid to precipitate Int. 3C29 (S)-3,5-difluoro-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzoic acid (0.20 g).

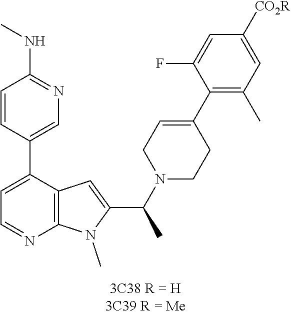

Int. 3C38. (S)-3-fluoro-5-methyl-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzoic acid PdDPPFCl₂-DCM (84 mg), Int. 1AF86 (1.0 g), N-methyl-5-(4,4,5,5-tetra-methyl-1,3,2-dioxaborolan-2-yl)pyridin-2-amine (0.63 g), and NaHCO₃ (0.60 g) were stirred 16 h in dioxane/H₂O (4:1, 10 mL) at 80° C. under an inert atmosphere. The mixture was filtered. The OL (water/EtOAc) was dried, filtered, and concentrated to give Int. 3C39 methyl (S)-3-fluoro-5-methyl-4-(1-(1-(1-methyl-4-(6-(methyl-amino)pyridin-3-yl)-1H-pyrrolo[2,3-b]pyridin-2-yl)ethyl)-1,2,3,6-tetrahydro-pyridin-4-yl)-benzoate (1.2 g). LiOH—H₂O (0.38 g) and Int. 3C39 (0.95 g) were stirred 6 h in THF/MeOH/H₂O (2:2:1, 25 mL) at 0° C. to 60° C. The residue after concentration was stirred in aq. HCl to precipitate Int. 3C38 (0.80 g).

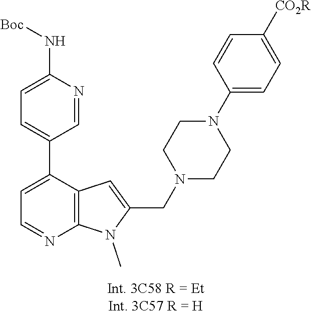

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl)carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)benzoate (2 g). 6M HCl (20 mL) was added drop-wise at 0° C. to a solution of Int. 3C58 (2.1 g) in dioxane (15 mL) and stirred for 6 h at 85° C. The mixture was concentrated and triturated in Et₂O to give Int. 3C57 (1.5 g).

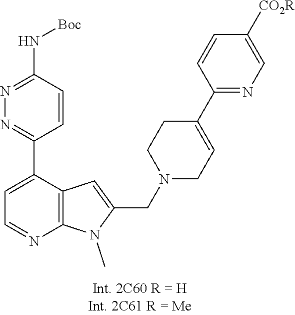

Int. 2C62 (0.30 g), tert-butyl (6-bromopyridazin-3-yl)carbamate (0.14 g), K₂CO₃ (0.19 g) and PdDPPFCl₂-DCM (37 mg) were stirred ON in dioxane/water (4:1, 10 mL) at 80° C. under an inert atmosphere. The OL (water/EtOAc) was dried, filtered, concentrated, and HPLC-purified to give Int. 2C61 methyl 1′-((4-(6-((tert-butoxycarbonyl)-amino)-pyridazin-3-yl)-1-methyl-1H-pyrrolo[2,3-b]pyridin-2-yl)methyl)-1′,2′,3′,6′-tetrahydro-[2,4′-bipyridine]-5-carboxylate (0.20 g). LiOH—H₂O (19 mg) and Int. 2C61 (25 mg) were stirred ON in THF/H₂O (1:1, 4 mL). Et₃N HCl (92 mg) was added. The mixture was concentrated to give Int. 2C60 1′-((4-(6-((tert-butoxycarbonyl)amino)pyridazin-3-y)-1-methyl-1I-pyrrolo[2,3-b]pyridin-2-yl)methyl)-1′,2′,3′,6′-tetrahydro-[2,4-bipyridine]-5-carboxylic acid (24 mg).

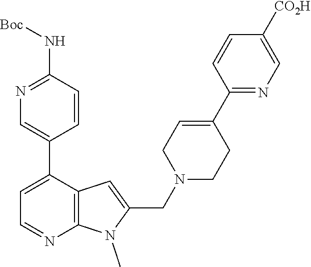 [SMILES: Cn1c(CN2CC=C(c3ccc(C(=O)O)cn3)CC2)cc2c(-c3ccc(NC(=O)OC(C)(C)C)nc3)ccnc21]

Int. 2C68 (285 mg), tert-butyl (5-(4,4,5,5-tetramethyl-1,3,2-dioxaborolan-2-yl)pyridin-2-yl)carbamate (227 mg), K₂CO₃ (268 mg) and PdDPPFCl₂-DCM (53 mg) were stirred ON in dioxane/H₂O (4:1, 10 mL) at 80° C. under an inert atmosphere. The OL (water/EtOAc) was dried, filtered, concentrated, and HPLC-purified to give Int. 2C67 methyl 1′-((4-(6-((tert-butoxycarbonyl)amino)pyridin-3-yl)-1-methyl-1H-pyrrolo[2,3-b]pyridin-2-yl)methyl)-1′,2′,3′,6′-tetrahydro-[2,4′-bipyridine]-5-carboxylate (57 mg). Int. 2C66 1′-((4-(6-((tert-butoxycarbonyl)amino)pyridin-3-yl)-1-methyl-1H-pyrrolo[2,3-b]pyridin-2-yl)methyl)-1′,2′,3′,6′-tetrahydro-[2,4′-bipyridine]-5-carboxylic acid (19 mg) was prepared similarly to Int. 2C60 from Int. 2C67 (20 mg).

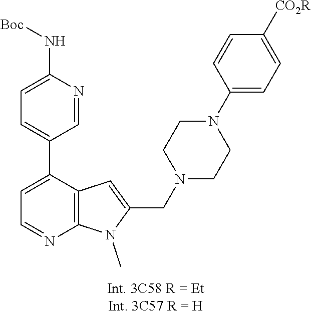

PdDPPFCl₂-DCM (0.20 g) was added to a mixture of Int. 3C59 (2.2 g), tert-butyl (5-(4,4,5,5-tetramethyl-1,3,2-dioxaborolan-2-yl)pyridin-2-yl)carbamate (2.0 g) and NaHCO₃ (0.81 g) in dioxane/H₂O (3:1, 24 mL) and stirred for 16 h at 85° C. under an inert atmosphere and filtered. The OL (water/EtOAc) was dried, filtered, concentrated and purified by FC (PE/EtOAc 2:1 to 1:1) to give Int. 3C58 ethyl 4-(4-((4-(6-((tert-butoxycarbonyl)amino)pyridin-3-yl)-1-methyl-1H-pyrrolo[2,3-b]pyridin-2-yl)methyl)piperazin-1-yl)-benzoate (2 g). 6M HCl (20 mL) was added drop wise at 0° C. to a solution of Int. 3C58 (2.1 g) in dioxane (15 mL) and stirred for 6 h at 85° C. The mixture was concentrated and triturated in Et₂O to give Int. 3C57 4-(4-((4-(6-aminopyridin-3-yl)-1-methyl-1H-pyrrolo[2,3-b]pyridin-2-yl)methyl)piperazin-1-yl)benzoic acid (1.5 g).

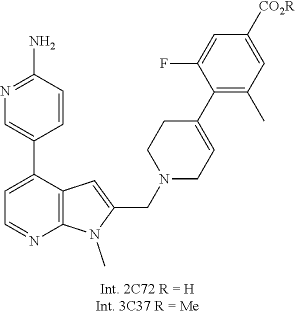

Int. 3C73 (150 mg), N-2-pyridine-5-boronic acid pinacol ester (85 mg), K₂CO₃ (121 mg) and PdDPPFCl₂-DCM (26 mg) were stirred in dioxane/H₂O (4:1, 2.5 mL) ON at 100° C. under an inert atmosphere. The OL (water/EtOAc) was dried, filtered, concentrated and HPLC-purified to give Int. 3C37 methyl 4-(1-((4-(6-aminopyridin-3-yl)-1-methyl-1H-pyrrolo[2,3-b]pyridin-2-yl)methyl)-1,2,3,6-tetrahydropyridin-4-yl)-3-fluoro-5-methylbenzoate (47 mg). LiOH—H₂O (9 mg) and Int. 3C37 (10 mg) were stirred ON in THF/H₂O (1:1, 4 mL). Et₃N—HCl (43 mg) was added and the mixture was concentrated to give Int. 2C72 4-(1-((4-(6-aminopyridin-3-yl)-1-methyl-1H-pyrrolo[2,3-b]pyridin-2-yl)methyl)-1,2,3,6-tetrahydropyridin-4-yl)-3-fluor-5-methylbenzoic acid (10 mg).

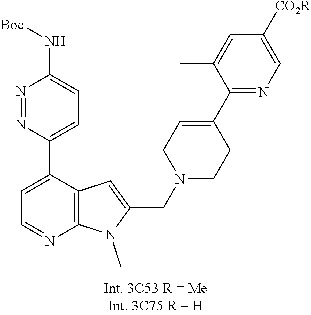

Int. 2C76 (0.30 g), tert-butyl (6-bromopyridazin-3-yl)carbamate (0.10 g), K₂CO₃ (0.30 mg) and PdDPPFCl₂-DCM (20 mg) were stirred in dioxane/water (4:1, 5 mL) ON at 80° C. under an inert atmosphere. The OL (water/EtOAc) was dried, filtered, concentrated, and HPLC-purified to give Int. 3C53 methyl 1′-((4-(6-((tert-butoxycarbonyl)amino)pyridazin-3-yl)-1-methyl-1H-pyrrolo[2,3-b]pyridin-2-yl)methyl)-3-methyl-1′,2′,3′,6′-tetrahydro-[2,4′-bipyridine]-5-carboxylate (0.14 g). Int. 2C75 1′-((4-(6-((tert-butoxycarbonyl)-amino)pyridazin-3-yl)-1-methyl-1H-pyrrolo[2,3-b]pyridin-2-yl)-methyl)-3-methyl-1′,2′,3′,6′-tetrahydro-[2,4′-bipyridine]-5-carboxylic acid (20 mg) was prepared similarly to Int. 2C72 from Int. 3C53 (20 mg).

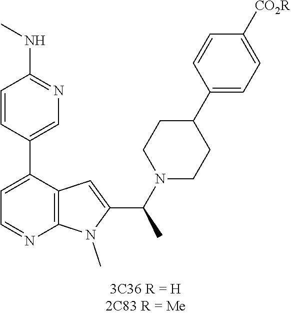

Int. 3E35 (3. 0 g), N-methyl-5-(4,4,5,5-tetramethyl-1,3,2-dioxaborolan-2-yl)pyridin-2-amine (1.8 g), NaHCO₃ (1.1 g), and PdDPPFCl₂-DCM (0.27 g) were stirred 16 h in dioxane/H₂O (4:1, 30 mL) at 85° C. under an inert atmosphere. The OL (water/EtOAc) was dried, filtered, concentrated, and purified by FC (PE/EtOAc 1:1 to 2:3) to give Int. 2C83 methyl (S)-4-(1-(1-(1-methyl-4-(6-(methylamino)-pyridin-3-yl)-1H-pyrrolo[2,3-b]pyridin-2-yl)ethyl)piperidin-4-yl)benzoate (3.0 g). LiOH—H₂O (0.89 g) and Int. 2C83 (2.1 g) were stirred 16 h in THF/MeOH/H₂O (2:2:1, 10 mL) at 0° C. to RT. The residue after concentration was stirred in water (pH adjusted aq. citric acid), filtered, and dried to give Int. 3C36 (S)-4-(1-(1-(1-methyl-4-(6-(methylamino)pyridin-3-yl)-1H-pyrrolo[2,3-b]pyridin-2-yl)ethyl)piperidin-4-yl)benzoic acid (1.9 g).

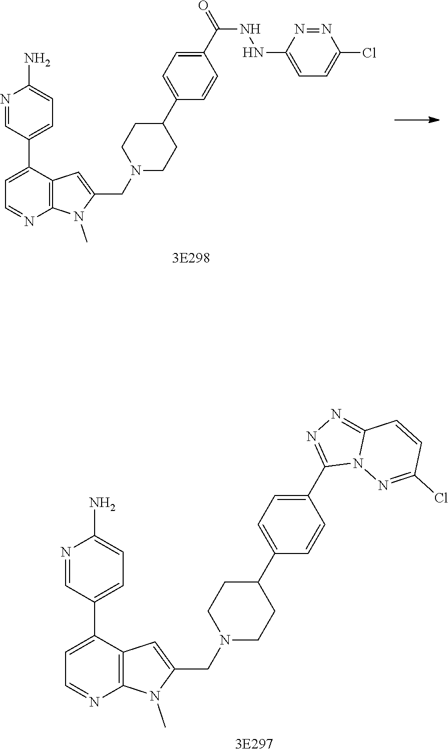 [SMILES: Cn1c(CN2CCC(c3ccc(C(=O)NNc4ccc(Cl)nn4)cc3)CC2)cc2c(-c3ccc(N)nc3)ccnc21 ; Cn1c(CN2CCC(c3ccc(-c4nnc5ccc(Cl)nn45)cc3)CC2)cc2c(-c3ccc(N)nc3)ccnc21]

Int. 3E23 (0.17 g) and HATU (0.2 g) were stirred 4 h in DMF/DIPEA (5.6:1, 1.8 mL). 3-Chloro-6-hydrazineyl-pyridazine (67 mg) was added and stirring continued ON. The OL (2MeTHF/water) was washed with brine, dried, concentrated, and purified by FC (DCM/MeOH/Et₃N 1:0:0 to 90:8.5:0.5) to give Int. 3E298 4-[1-[[4-(6-amino-3-pyridyl)-1-methyl-pyrrolo[2,3-b]-pyridin-2-yl]methyl]-4-piperidyl]-N′-(6-chloropyridazin-3-yl)benzohydrazide (0.12 g). This material was stirred 3 h in AcOH (4 mL) at 90° C., concentrated, and purified by FC (DCM/MeOH/Et₃N 95:4.5:0.5) to give Int. 101/3E297 5-[2-[[4-[4-(6-chloro-[1,2,4]triazolo[4,3-b]pyridazin-3-yl)phenyl]-1-piperidyl]-methyl]-1-methyl-pyrrolo[2,3-b]pyridin-4-yl]pyridin-2-amine (77 mg).

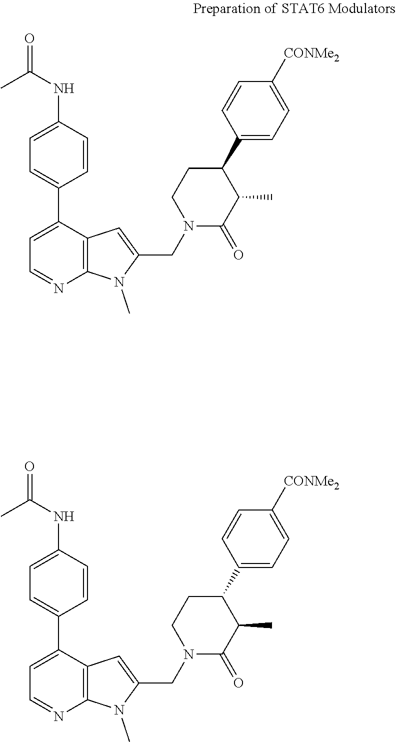

### Example 1aa2 and 1aa2′. N,N-Dimethyl-4-[(3S,4R)-1-[[4-(4-acetamidophenyl)-1-methyl-pyrrolo[2,3-b]pyridin-2-yl]methyl]-3-methyl-2-oxo-4-piperidyl]benzamide and N,N-dimethyl-4-[(3R,4S)-1-[[4-(4-acetamidophenyl)-1-methyl-pyrrolo[2,3-b]pyridin-2-yl]methyl]-3-methyl-2-oxo-4-piperidyl]benzamide

Int. 1A10 (30 mg), (4-acetamidophenyl)boronic acid (0.11 g), PdDPPFCl₂-DCM (34 mg), and NaHCO₃ (84 mg) were degassed in dioxane/water (10:1, 1.1 mL) and stirred 1.5 h at 105° C. under MW conditions. The mixture was filtered and mixed with another batch prepared on the same scale and purified by FC (EtOAc/hexane 7:3) to give Examples 1aa2/1aa2′. This mixture was resolved by HPLC using a Chiralpak IC 250×4.6 mm 5 μm column and an eluent of MeOH/DCM 9:1 at a (1.0 mL/min) to give Example 1aa2 (25 mg, first peak). The second peak was purified again by SFC using a TORUS 1-AA 250×4.6 5 μm column operated at 30° C. with an eluent of 70% CO₂ and 30% MeOH (3 g/min) and a back pressure of 1500 psi to give Example 1aa2′ (20 mg). The absolute configurations of these compounds were not determined.

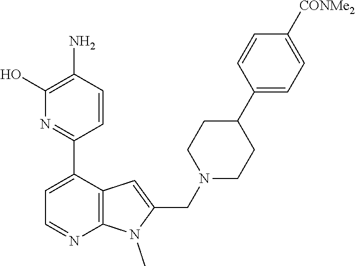

### Example 1ad1. 4-(1-((4-(5-Amino-6-hydroxypyridin-2-yl)-1-methyl-1H-pyrrolo[2,3-b]pyridin-2-yl)methyl)piperidin-4-yl)-N,N-dimethylbenzamide

Int. 1AB4 (20 mg), PdDPPFCl₂-DCM (7 mg), 3-amino-6-bromopyridin-2-ol (24 mg, HBr salt), and K₂CO₃ (28 mg) were degassed in dioxane/water (5:1, 1.2 mL) and stirred 1 h at 100° C. The mixture was filtered and purified by HPLC to give Example 1ad1 (10 mM in DMSO (1.05 mL)).

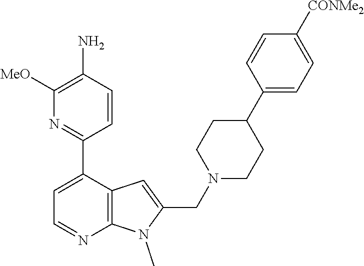

### Example 1ad2. 4-(1-((4-(5-Amino-6-methoxypyridin-2-yl)-1-methyl-1H-pyrrolo[2,3-b]pyridin-2-yl)methyl)piperidin-4-yl)-N,N-dimethylbenzamide

### Example 1ad2 (10 mM in DMSO (1.16 mL)) was prepared similarly to Example 1ad1 from 6-bromo-2-methoxypyridin-3-amine (24 mg)

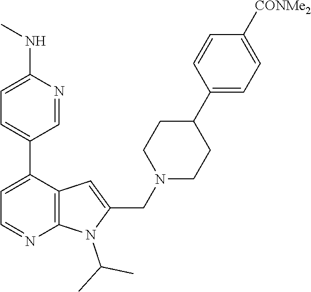

### Example 1ad3. 4-[1-[[1-iso-Propyl-4-[6-(methylamino)-3-pyridyl]pyrrolo[2,3-b]pyridin-2-yl]methyl]-4-piperidyl]-N,N-dimethyl-benzamide

Int. 1AD4 (50 mg) was stirred 4 h in DCM/4M HCl in dioxane (30:1, 3 mL) at 0° C. to RT, concentrated, and HPLC-purified give Example 1ad3 (23 mg).

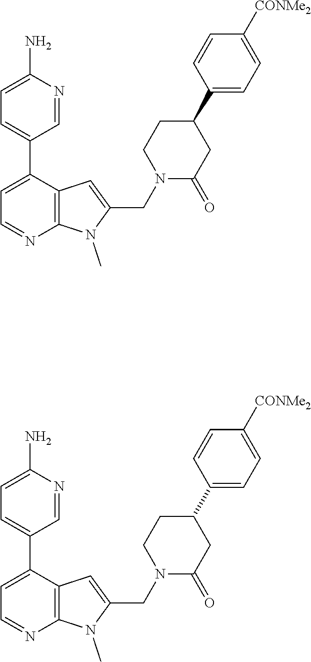

### Examples 1ae4 and 1ae5. (R)—N,N-Dimethyl-4-[1-[[4-(6-amino-3-pyridyl)-1-methyl-pyrrolo[2,3-b]pyridin-2-yl]methyl]-2-oxo-4-piperidyl]benzamide and (S)—N,N-dimethyl-4-[1-[[4-(6-amino-3-pyridyl)-1-methyl-pyrrolo[2,3-b]pyridin-2-yl]methyl]-2-oxo-4-piperidyl]benzamide

Int. 1AB3 (20 mg), PdDPPFCl₂-DCM (3 mg), 5-(4,4,5,5-tetramethyl-1,3,2-dioxaborolan-2-yl)pyridin-2-amine (14 mg), and Na₂CO₃ (14 mg) were degassed in DMF/water (5:1, 1.2 mL) and stirred ON at 100° C., filtered, and HPLC-purified to give Example 1ae4 (52 mM in DMSO (0.5 mL)). Example 1ae5 (10 mM in 1.1 mL DMSO) was prepared similarly from 1AB2 (16 mg) and 5-(4,4,5,5-tetramethyl-1,3,2-dioxaborolan-2-yl)pyridin-2-amine (10 mg). The absolute configurations of these compounds were not determined.

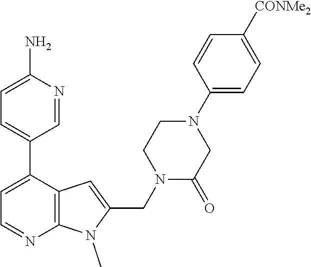

### Example 1ae7. 4-[4-[[4-(6-Amino-3-pyridyl)-1-methyl-pyrrolo[2,3-b]pyridin-2-yl]methyl]-3-oxo-piperazin-1-yl]-N,N-dimethyl-benzamide

Int. 1AE16 (0.10 g), 5-(4,4,5,5-tetramethyl-1,3,2-dioxaborolan-2-yl)pyridin-2-amine (70 mg), NaHCO₃ (54 mg), and PdDPPFCl₂-DCM (17 mg) were degassed in dioxane/water (10:1, 3.3 mL) and stirred 1.5 h at 130° C., concentrated, and purified by FC (DCM/MeOH 9:1) to give Example 1ae7 (35 mg).

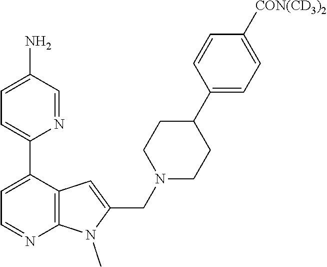

### Example 1ae10. 4-[1-[[4-(5-Amino-2-pyridyl)-1-methyl-pyrrolo[2,3-b]pyridin-2-yl]methyl]-4-piperidyl]-N,N-bis(trideuteriomethyl)benzamide

Int. 1D8 (30 mg), 6-bromopyridin-3-amine (12 mg), and PdDPPFCl₂-DCM (3 mg) were degassed in DMF (3 mL) and water (0.75 mL containing 11 mg Na₂CO₃) and stirred ON at 120° C., filtered, and HPLC-purified to give Example 1ae10 (4.76 mM in DMSO (0.70 mL)).

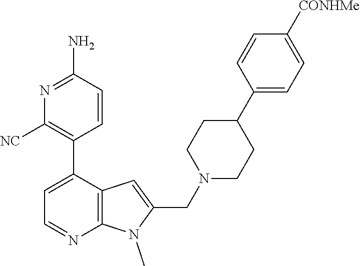

### Example 1ae14. 4-[1-[[4-(6-Amino-2-cyano-3-pyridyl)-1-methyl-pyrrolo[2,3-b]pyridin-2-yl]methyl]-4-piperidyl]-N-methyl-benzamide

Int. 1AE36 (0.12 g), B₂Pin₂ (0.11 g), KOAc (61 mg), and PdDPPFCl₂-DCM (17 mg) were degassed in DMSO (3 mL) and stirred ON at 100° C. The OL (water/DCM) was dried and concentrated. The residue, 6-amino-3-bromo-picolinonitrile (28 mg), K₂CO₃ (58 mg), and PdDPPFCl₂-DCM (11 mg) were degassed in DMF/water (5:1, 2.4 mL) and stirred 2 h at 100° C. The OL (water/DCM) was dried, concentrated, and purified by FC (DCM/MeOH 1:0 to 93:7) to give 4-(1-((4-(6-amino-2-cyanopyridin-3-yl)-1-methyl-1H-pyrrolo[2,3-b]pyridin-2-yl)methyl)piperidin-4-yl)-N-(3,4-dimethoxybenzyl)-N-methylbenzamide. 65 mg of this material was stirred ON in DCM/TFA (1:2, 3 mL), concentrated, and HPLC-purified to give Example 1ae14 (6.5 mg).

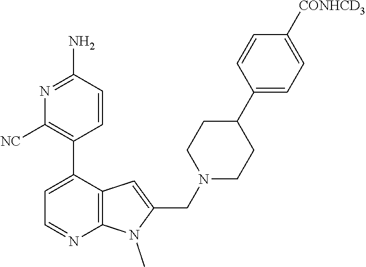

### Example 1ae15. 4-[1-[[4-(6-Amino-2-cyano-3-pyridyl)-1-methyl-pyrrolo[2,3-b]pyridin-2-yl]methyl]-4-piperidyl]-N-(trideuteriomethyl)benzamide

Int. 1AC4 (0.10 g), HATU (0.16 g), and DIPEA (0.19 mL) were stirred in DMF (3 mL) for 2 min. NH₂CD₃ (23 mg, HCl salt) was added and stirring continued 2 h. The OL (sat. aq. NaHCO₃/DCM) was dried, concentrated, and purified by FC (DCM/MeOH 1:0 to 9:1) to give 4-[1-[(4-bromo-1-methyl-pyrrolo[2,3-b]pyridin-2-yl)-methyl]-4-piperidyl]-N-(trideuteriomethyl)benzamide (88 mg). This material, PdDPPFCl₂-DCM (16 mg), B₂Pin₂ (0.15 g), and KOAc (77 mg) were degassed in DMSO (2.5 mL) and stirred 3 h at 100° C. The OL (water/DCM) was dried and concentrated. The residue, 6-amino-3-bromopicolinonitrile (20 mg), K₂CO₃ (41 mg), and PdDPPFCl₂-DCM (8 mg) were degassed in DMF/water (5:1, 1.8 mL) and stirred 1 h at 100° C. The OL (water/DCM) was dried, concentrated, and purified by FC (DCM/MeOH 1:0 to 9:1) and by HPLC to give Example 1ae15 (2.89 mM in DMSO (0.70 mL)).

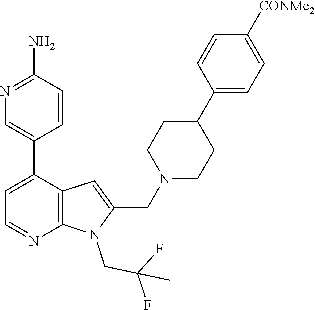

### Example 1ae19. 4-[1-[[1-(2,2-Difluoropropyl)-4-[6-(methylamino)-3-pyridyl]pyrrolo[2,3-b]pyridin-2-yl]methyl]-4-piperidyl]-N,N-dimethyl-benzamide

Int. 1AE32 (0.15 g), Cs₂CO₃ (0.43 g), and 2,2-difluoropropyl 4-methylbenzene-sulfonate (0.66 g) were stirred ON in DMF (15 mL) at 0° C. to 80° C. The OL (water/EtOAc) was dried, concentrated, and purified by FC (EtOAc/hexane 4:1) to give tert-butyl N-[5-[1-(2,2-difluoropropyl)-2-[[4-[4-(dimethyl-carbamoyl)phenyl]-1-piperidyl]methyl]-pyrrolo-[2,3-b]pyridin-4-yl]-2-pyridyl]-N-methyl-carbamate (50 mg). This material was stirred 4 h in DCM/TFA (167:1, 5 mL) at 0° C. to RT, concentrated, and HPLC-purified to give Example 1ae19 (10 mg).

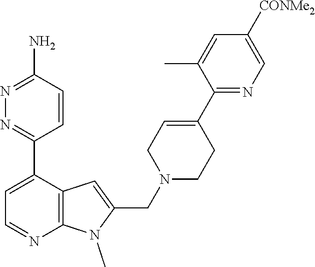

### Example 1ae22. 1′-((4-(6-Aminopyridazin-3-yl)-1-methyl-1H-pyrrolo[2,3-b]pyridin-2-yl)methyl)-N,N,3-trimethyl-1′,2′,3′,6′-tetrahydro-[2,4′-bipyridine]-5-carboxamide

Int. 1AE23 (2.0 g), tert-butyl (6-bromo-3-pyridazinyl)carbamate (2.0 g), PdDPPFCl₂-DCM (0.3 g), and K₂CO₃ (3.0 g) were degassed in dioxane (30 mL) and stirred ON at 80° C. The OL (EtOAc/water) was dried, and concen-trated to give tert-butyl N-[6-[2-[[4-[5-(dimethylcarbamoyl)-3-methyl-2-pyridyl]-3,6-dihydro-2H-pyridin-1-yl]-methyl]-1-methyl-pyrrolo[2,3-b]pyridin-4-yl]pyridazin-3-yl]carbamate (1.5 g). 4.5 g of this material was stirred ON in MeOH/4M HCl in dioxane (6.7:1, 46 mL), concentrated, and HPLC-purified to give Example 1ae22 (0.4 g).

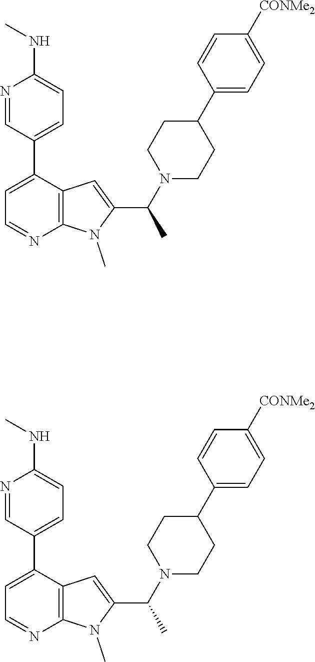

### Examples 1af7 and 1af8. (S)—N,N-Dimethyl-4-(1-(1-(1-methyl-4-(6-(methylamino)pyridin-3-yl)-1H-pyrrolo[2,3-b]pyridin-2-yl)ethyl)piperidin-4-yl)benzamide and (R)—N,N-Dimethyl-4-(1-(1-(1-methyl-4-(6-(methylamino)pyridin-3-yl)-1H-pyrrolo[2,3-b]pyridin-2-yl)ethyl)piperidin-4-yl)benzamide

Int. 1AF17 (0.12 g), tert-butyl N-methyl-N-[5-(4,4,5,5-tetramethyl-1,3,2-dioxaborolan-2-yl)-2-pyridyl]carbamate (94 mg), K₂CO₃ (81 mg), and PdDPPFCl₂-DCM (21 mg) were degassed in dioxane/water (4:1, 3 mL) and stirred 1 h at 100° C. under MW conditions. The mixture was filtered, concentrated, and purified by FC (DCM/MeOH 98:2) to give tert-butyl N-[5-[2-[1-[4-[4-(dimethylcarbamoyl)phenyl]-1-piperidyl]ethyl]-1-methyl-pyrrolo[2,3-b]pyridin-4-yl]-2-pyridyl]-N-methyl-carbamate (80 mg). This material was stirred ON in DCM/TFA (80:1, 2.1 mL), concentrated, and HPLC-purified to give N,N-dimethyl-4-[1-[1-[1-methyl-4-[6-(methyl-amino)-3-pyridyl]-pyrrolo[2,3-b]pyridin-2-yl]ethyl]-4-piperidyl]benzamide (38 mg). This material was purified by SFC using a Chiralcel OJ-H 250×10 5 μm column operated at 30° C. and an eluent of 70% CO₂ and 30% 30 mM NH₃ in MeOH (15 g/min) and a back pressure of 100 bar to give Example 1af7 (11 mg, first peak) and Example 1af8 (11 mg, second peak). The absolute configurations of these compounds were not determined.

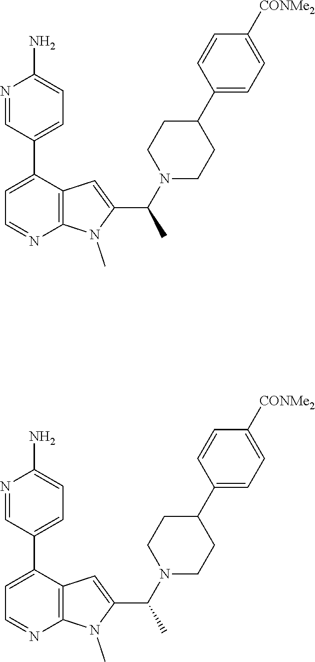

### Examples 1af1 and 1af12. (S)-4-(1-(1-(4-(6-amino-pyridin-3-yl)-1-methyl-1H-pyrrolo[2,3-b]pyridin-2-yl)ethyl)-piperidin-4-yl)-N,N-dimethyl-benzamide and (R)-4-(1-(1-(4-(6-aminopyridin-3-yl)-1-methyl-1H-pyrrolo[2,3-b]pyridin-2-yl)ethyl)piperidin-4-yl)-N,N-dimethylbenzamide

Int. 1AF17 (40 mg) was resolved by SFC using a Chiralpak OD-H 250×30 5 μm column operated at 30° C. and an eluent of 60% CO₂ and 40% IPA (20 g/min) and a back pressure of 100 bar to give Example 1af1l (9 mg, first peak) and Example 1af12 (11 mg, second peak). The absolute configurations of these compounds were not determined.

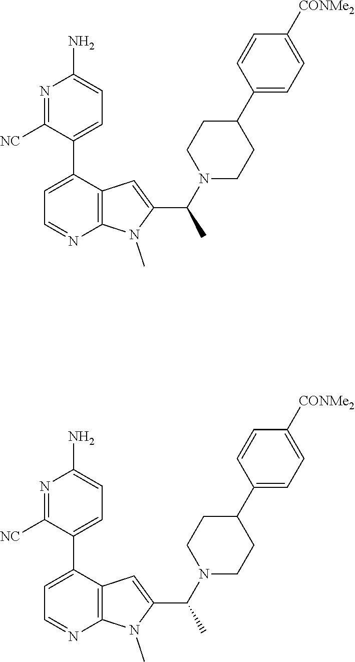

### Examples 1af13 and 1af14. (S)-4-(1-(1-(4-(6-amino-2-cyanopyridin-3-yl)-1-methyl-1H-pyrrolo[2,3-b]pyridin-2-yl)ethyl)piperidin-4-yl)-N,N-dimethylbenzamide and (R)-4-(1-(1-(4-(6-amino-2-cyanopyridin-3-yl)-1-methyl-1H-pyrrolo[2,3-b]pyridin-2-yl)ethyl)piperidin-4-yl)-N,N-dimethylbenzamide

Int. 1AF25 (22 mg) was resolved by SFC using a Chiralcel OJ-H 250×30 5 μm column operated at 30° C. and an eluent of 70% CO₂ and 30% 30 mM NH₃ in MeOH (15 g/min) and a back pressure of 100 bar to give Example 1af13 (5.1 mg, first peak) and Example 1af14 (5.1 mg, second peak). The absolute configurations of these compounds were not determined.

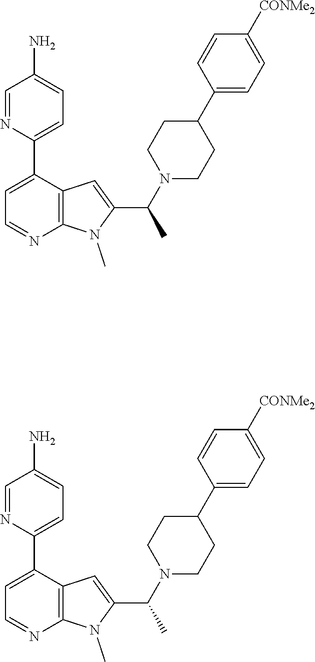

### Examples 1af15 and 1af16. (S)-4-(1-(1-(4-(5-amino-pyridin-2-yl)-1-methyl-1H-pyrrolo[2,3-b]pyridin-2-yl)-ethyl)piperidin-4-yl)-N,N-dimethylbenzamide and (R)-4-(1-(1-(4-(5-aminopyridin-2-yl)-1-methyl-1H-pyrrolo[2,3-b]pyridin-2-yl)ethyl)piperidin-4-yl)-N,N-dimethylbenzamide

Int. 1AF22 (80 mg) was resolved by SFC using a Chiral ART Amylose-C NEO 250×10 5 μm column operated at 30° C. and an eluent of 60% CO₂ and 40% MeOH containing 0.5% NH₃ (20 g/min) and a back pressure of 100 bar to give Example 1af15 (25 mg, first peak) and Example 1af16 (27 mg, second peak). The absolute configurations of these compounds were not determined.

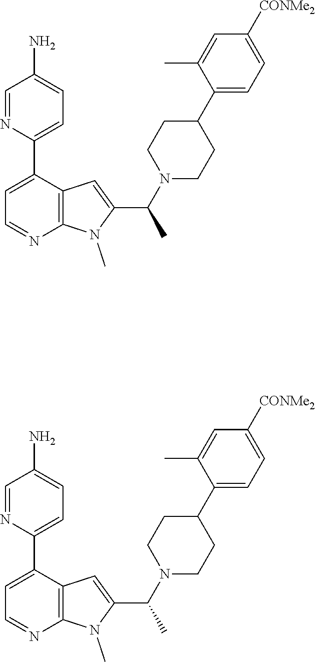

### Examples 1af17 and 1af18. (S)-4-(1-(1-(4-(5-aminopyridin-2-yl)-1-methyl-1H-pyrrolo[2,3-b]pyridin-2-yl)ethyl)piperidin-4-yl)-N,N,3-trimethylbenzamide and (R)-4-(1-(1-(4-(5-aminopyridin-2-yl)-1-methyl-1H-pyrrolo[2,3-b]pyridin-2-yl)ethyl)piperidin-4-yl)-N,N,3-trimethylbenzamide

Int. 1AF41 (60 mg) was resolved by SFC using a Chiral ART Amylose-C NEO 250×10 5 μm column operated at 30° C. and an eluent of 60% CO₂ and 40% 30 mM NH₃ in MeOH (15 g/min) and a back pressure of 100 bar to give Example 1af17 (16 mg, first peak) and Example 1af18 (15 mg, second peak). The absolute configurations of these compounds were not determined.

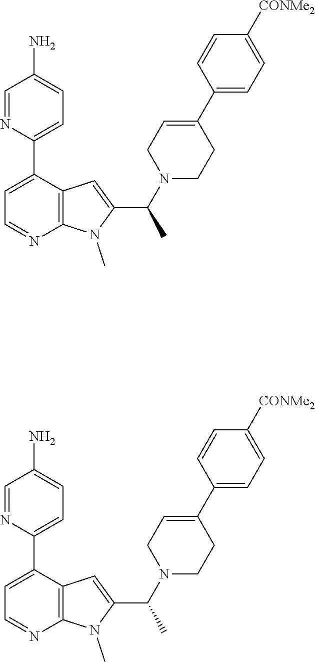

### Examples 1af19 and 1af20. (S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-dimethylbenzamide and (R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N-dimethylbenzamide

Int. 1AF31 (50 mg) was resolved by SFC using a Chiralpak IA 250×10 5 μm column operated at 30° C. and an eluent of MeOH (3 mL/min) and a back pressure of 100 bar to give Example 1af19 (13 mg, first peak) and Example 1af20 (15 mg, second peak). The absolute configurations of these compounds were not determined.

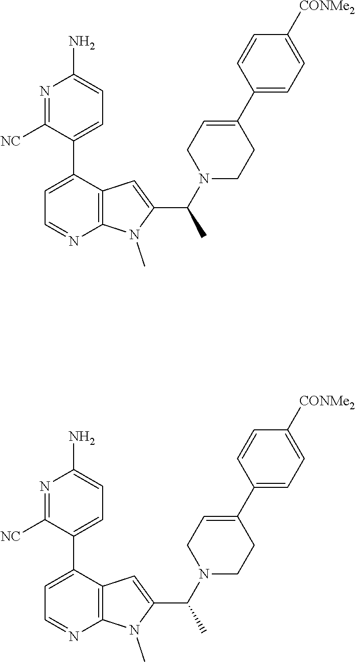

### Examples 1af21 and 1af23. (S)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide and (R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide

Int. 1AF35 (25 mg) was resolved by SFC using a Chiralpak AS-H 250×10 5 μm column operated at 30° C. and an eluent of 75% CO₂ and 25% MeOH containing 0.5% HNEt₂ (15 g/min) and a back pressure of 100 bar to give Example 1af21 (7 mg, first peak) and Example 1af23 (8 mg, second peak). The absolute configurations of these compounds were not determined.

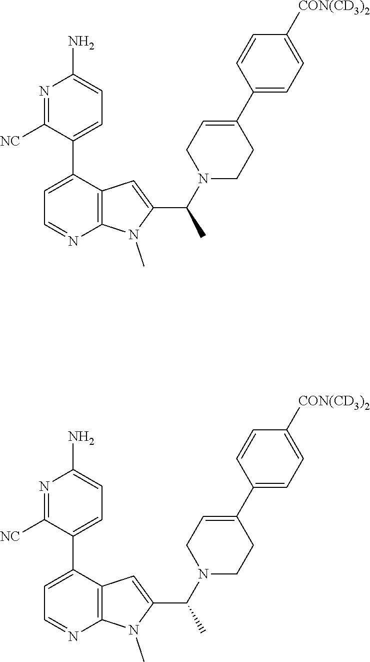

### Examples 1af22 and 1af24. (R)-4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide and (S)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide

Int. 1AF53 (55 mg) was resolved by SFC using a Chiralpak AS-H 250×10 5 μm column operated at 30° C. and an eluent of 80% CO₂ and 20% MeOH containing 0.5% HNEt₂ (20 g/min) and a back pressure of 100 bar to give Example 1af22 (20 mg, first peak) and Example 1af24 (18 mg, second peak). The absolute configurations of these compounds were not determined.

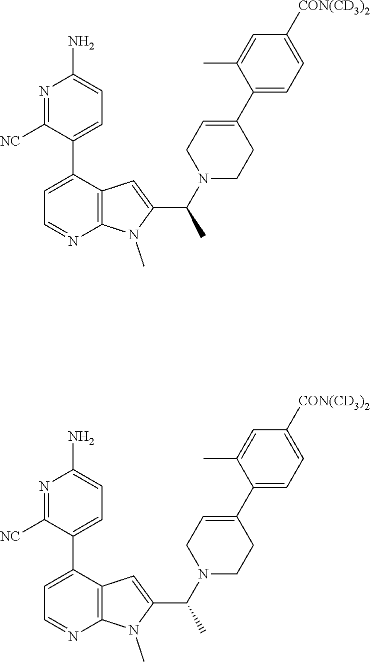

### Examples 1af25 and 1af26. (S)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide and (R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide

Int. 1AF59 (48 mg) was resolved by SFC using a Chiralcel OJ-H 250×10 5 μm column operated at 30° C. and an eluent of 80% CO₂ and 20% 30 mM NH₃ in MeOH (15 g/min) and a back pressure of 100 bar to give Example 1af25 (16 mg, first peak) and Example 1af26 (18 mg, second peak). The absolute configurations of these compounds were not determined.

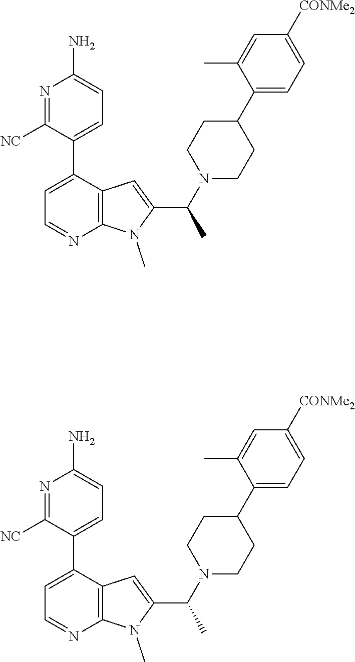

### Examples 1af27 and 1af28. (S)-4-(1-(1-(4-(6-Amino-2-cyanopyridin-3-yl)-1-methyl-1H-pyrrolo[2,3-b]pyridin-2-yl)ethyl)piperidin-4-yl)-N,N,3-trimethylbenzamide and (R)-4-(1-(1-(4-(6-amino-2-cyanopyridin-3-yl)-1-methyl-1H-pyrrolo[2,3-b]pyridin-2-yl)ethyl)piperidin-4-yl)-N,N,3-trimethylbenzamide

Int. 1AF45 (40 mg) was resolved by SFC using a Chiralpak AD-H 250×10 5 μm column operated at 30° C. and an eluent of 75% CO₂ and 25% 30 mM NH₃ in MeOH (15 g/min) and a back pressure of 100 bar to give Example 1af27 (11 mg, first peak) and Example 1af28 (12 mg, second peak). The absolute configurations of these compounds were not determined.

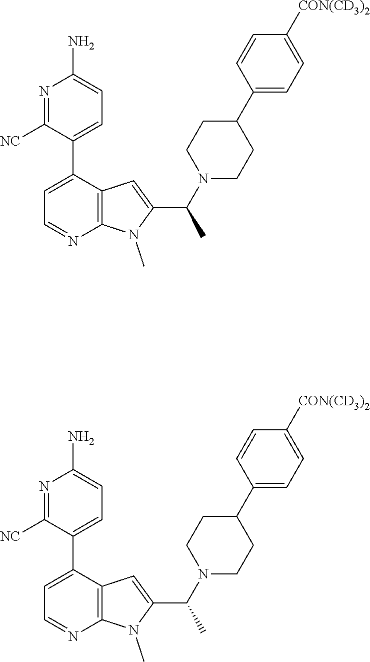

### Examples 1af29 and 1af30. (S)-4-(1-(1-(4-(6-Amino-2-cyanopyridin-3-yl)-1-methyl-1H-pyrrolo[2,3-b]pyridin-2-yl)ethyl)-piperidin-4-yl)-N,N-bis(methyl-d₃)benzamide and (R)-4-(1-(1-(4-(6-amino-2-cyanopyridin-3-yl)-1-methyl-1H-pyrrolo[2,3-b]pyridin-2-yl)ethyl)piperidin-4-yl)-N,N-bis(methyl-d₃)benzamide

Int. 1AF37 (87 mg) was resolved by SFC using a Chiralcel OJ-H 250×3 5 μm column operated at 30° C. and an eluent of 70% CO₂ and 30% 30 mM NH₃ in MeOH (90 g/min) and a back pressure of 100 bar to give Example 1af29 (24 mg, first peak) and Example 1af30 (26 mg, second peak). The absolute configurations of these compounds were not determined.

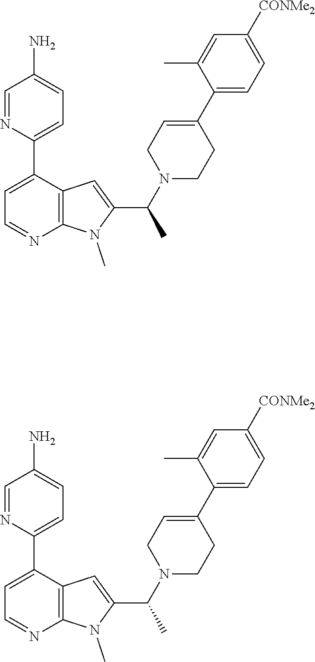

### Examples 1af32 and 1af34. (S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide and (R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide

Int. 1AF63 (38 mg) was resolved by SFC using a Chiralpak IA 250×10 5 μm column operated at 30° C. and an eluent of 60% CO₂ and 40% MeOH (100 g/min) and a back pressure of 100 bar to give Example 1af32 (12 mg, first peak) and Example 1af34 (14 mg, second peak). The absolute configurations of these compounds were not determined.

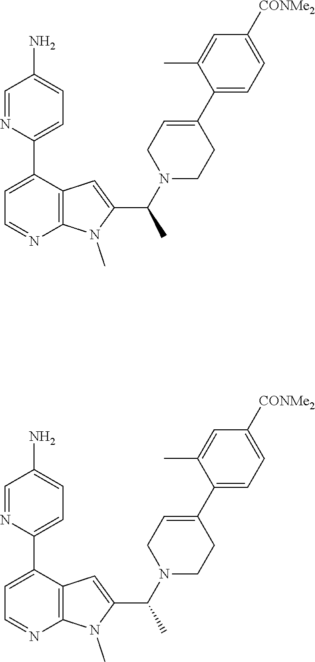

### Examples 1af33 and 1af31. (R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide and (S)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide

Int. 1AF51 (38 mg) was resolved by SFC using a Chiral ART Amylose-C NEO 250×10 5 μm column operated at 30° C. and an eluent of 60% CO₂ and 40% 30 mM NH₃ in MeOH (20 g/min) and a back pressure of 100 bar to give Example 1af33 (6 mg, first peak) and Example 1af31 (6 mg, second peak). The absolute configurations of these compounds were not determined.

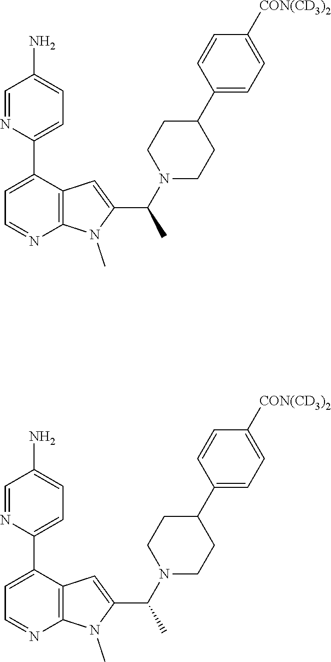

### Examples 1af35 and 1af36. (S)-4-(1-(1-(4-(5-Aminopyridin-2-yl)-1-methyl-1H-pyrrolo[2,3-b]pyridin-2-yl)-ethyl)piperidin-4-yl)-N,N-bis(methyl-d₃)benzamide and (R)-4-(1-(1-(4-(5-aminopyridin-2-yl)-1-methyl-1H-pyrrolo[2,3-b]pyridin-2-yl)ethyl)piperidin-4-yl)-N,N-bis(methyl-d₃)benzamide

Int. 1AF69 (80 mg) was resolved by SFC using a Chiral ART Amylose-C NEO 250×10 5 μm column operated at 30° C. and an eluent of 60% CO₂ and 40% 30 mM NH₃ in MeOH (20 g/min) and a back pressure of 100 bar to give Example 1af35 (11 mg, first peak) and Example 1af36 (16 mg, second peak). The absolute configurations of these compounds were not determined.

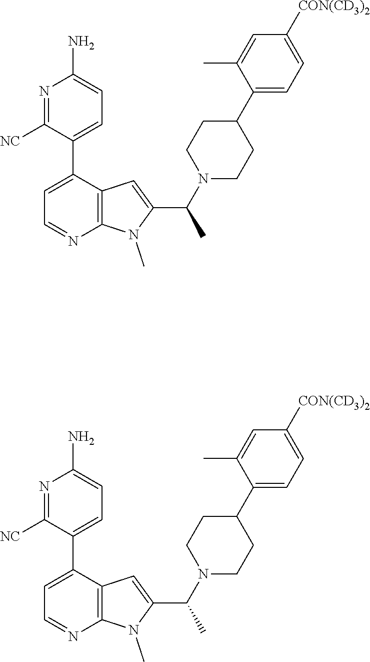

### Examples 1af37 and 1af38. (S)-4-(1-(1-(4-(6-Amino-2-cyanopyridin-3-yl)-1-methyl-1H-pyrrolo[2,3-b]pyridin-2-yl)ethyl)piperidin-4-yl)-3-methyl-N,N-bis(methyl-d₃)benzamide and (R)-4-(1-(1-(4-(6-amino-2-cyanopyridin-3-yl)-1-methyl-1H-pyrrolo[2,3-b]pyridin-2-yl)ethyl)-piperidin-4-yl)-3-methyl-N,N-bis(methyl-d₃)benzamide

Int. 1AF29 (57 mg) was resolved by SFC using a Chiral ART Amylose-C NEO 250×10 5 μm column operated at 30° C. and an eluent of 70% CO₂ and 30% 30 mM NH₃ in MeOH (20 g/min) and a back pressure of 100 bar to give Example 1af37 (25 mg, first peak) and Example 1af38 (20 mg, second peak). The absolute configurations of these compounds were not determined.

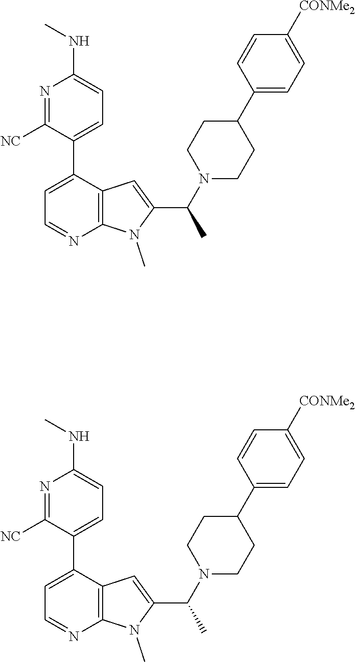

### Examples 1af39 and 1af40. (S)-4-(1-(1-(4-(2-xyano-6-(methylamino)pyridin-3-yl)-1-methyl-1H-pyrrolo-[2,3-b]pyridin-2-yl)ethyl)piperidin-4-yl)-N,N-dimethylbenzamide and (R)-4-(1-(1-(4-(2-cyano-6-(methyl-amino)pyridin-3-yl)-1-methyl-1H-pyrrolo[2,3-b]pyridin-2-yl)ethyl)piperidin-4-yl)-N,N-dimethylbenzamide

Int. 1AF67 (30 mg) was resolved by SFC using a Chiral ART Cellulose SZ 250×10 5 μm column operated at 30° C. and an eluent of 60% CO₂ and 40% MeOH containing 0.5% NH₃ (15 g/min) and a back pressure of 100 bar to give Example 1af39 (7 mg, first peak) and Example 1af40 (8 mg, second peak). The absolute configurations of these compounds were not determined.

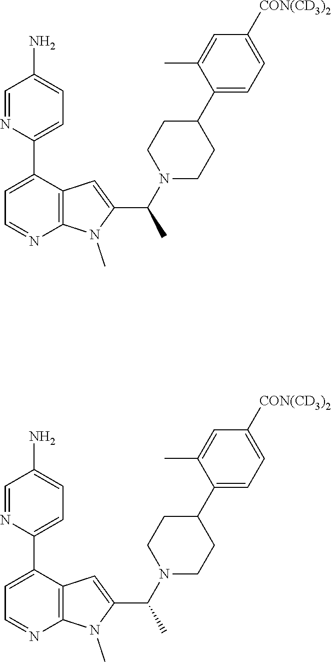

### Examples 1af41 and 1af42. (S)-4-(1-(1-(4-(5-Aminopyridin-2-yl)-1-methyl-1H-pyrrolo[2,3-b]pyridin-2-yl)ethyl)-piperidin-4-yl)-3-methyl-N,N-bis(methyl-d₃)benzamide and (R)-4-(1-(1-(4-(5-amino-pyridin-2-yl)-1-methyl-1H-pyrrolo[2,3-b]pyridin-2-yl)ethyl)piperidin-4-yl)-3-methyl-N,N-bis(methyl-d₃)benzamide

Int. 1AF57 (55 mg) was resolved by SFC using a Chiral ART Cellulose SZ 250×10 5 μm column operated at 30° C. and an eluent of 60% CO₂ and 40% 30 mM NH₃ in MeOH (15 g/min) and a back pressure of 100 bar to give Example 1af41 (5 mg, first peak) and Example 1af42 (8 mg, second peak). The absolute configurations of these compounds were not determined.

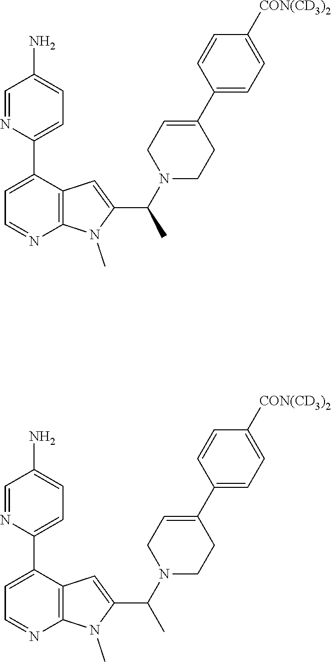

### Examples 1af43 and 1af46. (S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide and (R)-4-(1-(1-(4-(5-amino-pyridin-2-yl)-1-methyl-1H-pyrrolo[2,3-b]pyridin-2-yl)ethyl)-1,2,3,6-tetrahydro-pyridin-4-yl)-N,N-bis(methyl-d₃)benzamide

Int. 1AFF57 (30 mg) was resolved by SFC using a Chiralpak IA 250×10 5 μm column operated at 30° C. and an eluent of MeOH (3 mL/min) and a back pressure of 100 bar to give Example 1af43 (7 mg, first peak) and Example 1af46 (6 mg, second peak). The absolute configurations of these compounds were not determined.

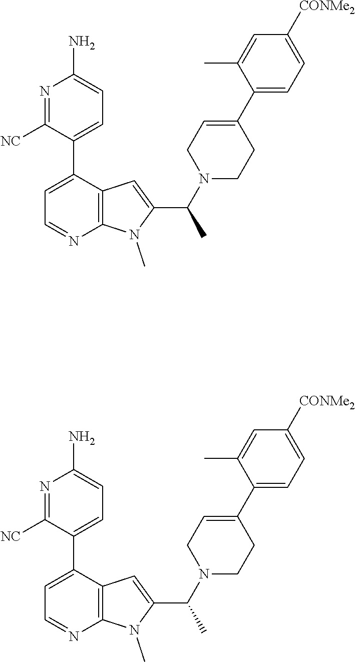

### Examples 1af44 and 1af45. (S)-4-(1-(1-(4-(6-Amino-2-cyanopyridin-3-yl)-1-methyl-1H-pyrrolo[2,3-b]-pyridin-2-yl)ethyl)-1,2,3,6-tetrahydropyridin-4-yl)-N,N,3-trimethylbenzamide and (R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N,3-trimethylbenzamide

Int. 1AF65 (22 mg) was resolved by SFC using a Chiralcel OJ-H 250×10 5 μm column operated at 30° C. and an eluent of 80% CO₂ and 20% 30 mM NH₃ in MeOH (20 g/min) and a back pressure of 100 bar to give Example 1af44 (10 mg, first peak) and Example 1af45 (8 mg, second peak). The absolute configurations of these compounds were not determined.

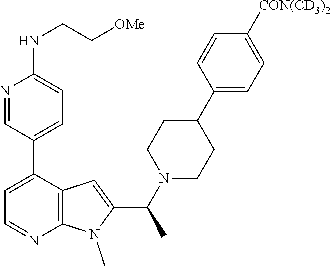

### Example 1af50. (S)-4-(1-(1-(4-(6-((2-methoxyethyl)amino)pyridin-3-yl)-1-methyl-1H-pyrrolo[2,3-b]pyridin-2-yl)ethyl)piperidin-4-yl)-N,N-bis(methyl-d₃)benzamide

Int. 1AF75 (60 mg), 5-bromo-N-(2-methoxyethyl)pyridin-2-amine (32 mg), K₂CO₃ (48 mg), and PdDPPFCl₂-DCM (9 mg) were degassed in dioxane/water 4:1 (4 mL) and stirred ON at 120° C. and filtered. The OL (water/5% MeOH in DCM) was dried, concentrated, and HPLC-purified to give Example 1af50 (10 mg).

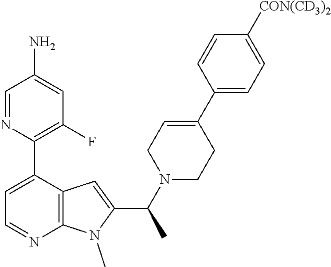

### Example 1af51. (S)-4-(1-(1-(4-(5-Amino-3-fluoropyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af51 (10 mg) was prepared similarly to Example 1af50 from 6-bromo-5-fluoropyridin-3-amine (26 mg).

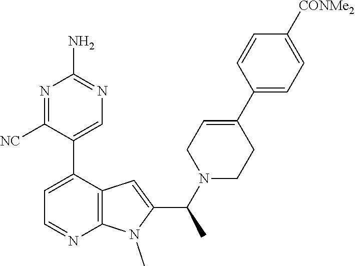

### Example 1af52. (S)-4-(1-(1-(4-(2-Amino-4-cyanopyrimidin-5-yl)-1-methyl-1H-pyrrolo[2,3-b]pyridin-2-yl)ethyl)-1,2,3,6-tetrahydropyridin-4-yl)-N,N-bis(methyl-d₃)benzamide

Int. 1AF76′ (70 mg), 2-amino-5-bromopyrimidine-4-carbonitrile (52 mg), K₃PO₄ (0.10 g), and BrettPhos Pd G4 (15 mg) were degassed in dioxane/water 4:1 (2 mL) and stirred 1 h at 100° C. under MW conditions and filtered. The OL (water/5% MeOH in DCM) was washed with dried, concentrated, and HPLC-purified to give Example 1af52 (33 mg).

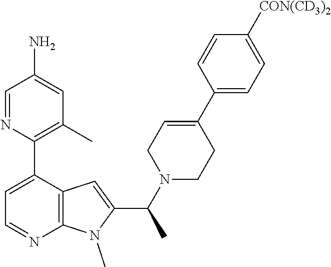

### Example 1af53. (S)-4-(1-(1-(4-(5-Amino-3-methylpyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af53 (12 mg) was prepared similarly to Example 1af50 from 6-bromo-5-methylpyridin-3-amine (26 mg).

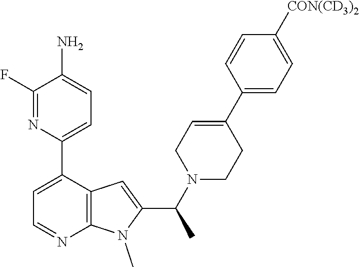

### Example 1af54. (S)-4-(1-(1-(4-(5-Amino-6-fluoropyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af54 (12 mg) was prepared similarly to Example 1af50 from 6-bromo-2-fluoropyridin-3-amine (31 mg).

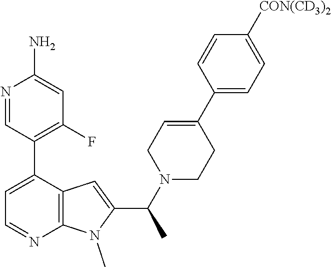

### Example 1af55. (S)-4-(1-(1-(4-(6-Amino-4-fluoropyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af55 (11 mg) was prepared similarly to Example 1af50 from 5-bromo-4-fluoropyridin-2-amine (31 mg).

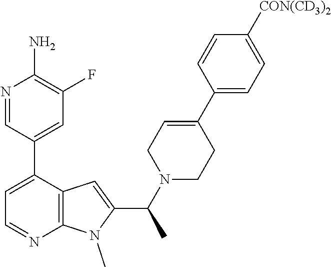

### Example 1af56. (S)-4-(1-(1-(4-(6-Amino-5-fluoropyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af56 (25 mg) was prepared similarly to Example 1af50 from 5-bromo-3-fluoro-pyridin-2-amine (29 mg).

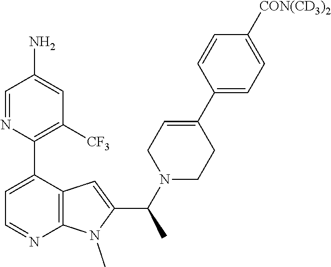

### Example 1af57. (S)-4-(1-(1-(4-(5-Amino-3-(trifluoromethyl)pyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af57 (13 mg) was prepared similarly to Example 1af50 from 6-bromo-5-(trifluoromethyl)pyridin-3-amine (39 mg).

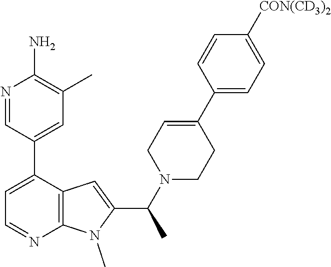

### Example 1af58. (S)-4-(1-(1-(4-(6-Amino-5-methylpyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af58 (13 mg) was prepared similarly to Example 1af50 from 5-bromo-3-methylpyridin-2-amine (31 mg).

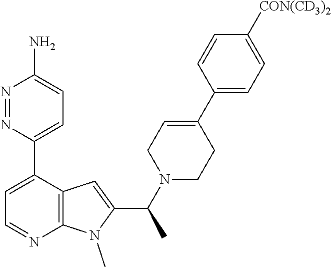

### Example 1af59. (S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-N,N-bis(methyl-d₃)benzamide

Example 1af58 (9 mg) was prepared similarly to Example 1af50 from 6-bromopyridazin-3-amine (24 mg).

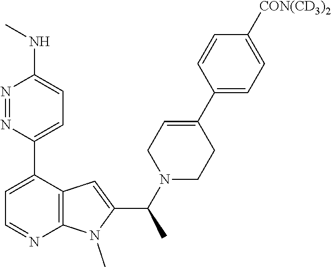

### Example 1af60. (S)—N,N-bis(methyl-d₃)-4-(1-(1-(1-methyl-4-(6-(methylamino)pyridazin-3-yl)-1H-pyrrolo[2,3-b]pyridin-2-yl)ethyl)-1,2,3,6-tetrahydropyridin-4-yl)benzamide

Example 1af60 (14 mg, TFA salt) was prepared similarly to Example 1af50 from 6-bromo-N-methylpyridazin-3-amine (31 mg).

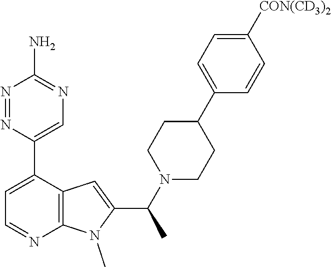

### Example 1af64. (S)-4-(1-(1-(4-(3-amino-1,2,4-triazin-6-yl)-1-methyl-1H-pyrrolo[2,3-b]pyridin-2-yl)ethyl)piperidin-4-yl)-N,N-bis(methyl-d₃)benzamide

Int. 1AF75 (70 mg), 6-bromo-1,2,4-triazin-3-amine (31 mg), BrettPhos Pd G4 (15 mg), and K₃PO₄ (0.10 g) were degassed in dioxane/water (4:1, 2 mL) and stirred 1 h at 100° C. under MW conditions. The mixture was filtered. The filtrate was partitioned between water and 5% MeOH in DCM. The OL was dried, concentrated, and HPLC-purified to give Example 1af64 (12 mg).

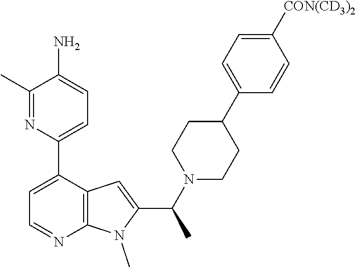

### Example 1af66. (S)-4-(1-(1-(4-(5-amino-6-methylpyridin-2-yl)-1-methyl-1H-pyrrolo[2,3-b]pyridin-2-yl)ethyl)piperidin-4-yl)-N,N-bis(methyl-d₃)benzamide

Int. 1AF75 (60 mg), 6-bromo-2-methylpyridin-3-amine (26 mg), PdDPPFCl₂-DCM (9 mg), and K₂CO₃ (48 mg) were degassed in dioxane/water (4:1, 4 mL) and stirred ON at 120° C. The mixture was filtered. The filtrate was partitioned between water and 5% MeOH in DCM. The OL was dried, concentrated, and HPLC-purified to give Example 1af66 (14 mg).

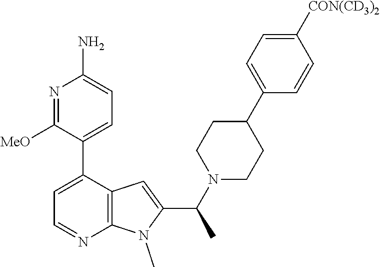

### Example 1af67. (S)-4-(1-(1-(4-(6-amino-2-methoxypyridin-3-yl)-1-methyl-1H-pyrrolo[2,3-b]pyridin-2-yl)ethyl)piperidin-4-yl)-N,N-bis(methyl-d₃)benzamide

Int. 1AF75 (60 mg), 5-bromo-6-methoxy-pyridin-2-amine (30 mg), PdDPPFCl₂-DCM (11 mg), and K₂CO₃ (94 mg) were degassed in dioxane/water (4:1, 2 mL) and stirred ON at 110° C. The mixture was filtered. The filtrate was partitioned between water and 5% MeOH in DCM. The OL was dried, concentrated, and HPLC-purified to give Example 1af67 (25 mg).

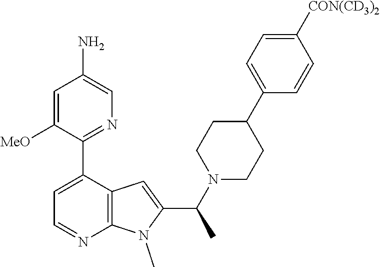

### Example 1af68. (S)-4-(1-(1-(4-(5-amino-3-methoxypyridin-2-yl)-1-methyl-1H-pyrrolo[2,3-b]pyridin-2-yl)ethyl)piperidin-4-yl)-N,N-bis(methyl-d₃)benzamide

Int. 1AF75 (60 mg), 6-bromo-5-methoxy-pyridin-3-amine (30 mg), PdDPPFCl₂-DCM (11 mg), and K₂CO₃ (94 mg) were degassed in dioxane/water (4:1, 2 mL) and stirred ON at 110° C. The mixture was filtered. The filtrate was partitioned between water and 5% MeOH in DCM. The OL was dried, concentrated, and HPLC-purified to give Example 1af68 (30 mg).

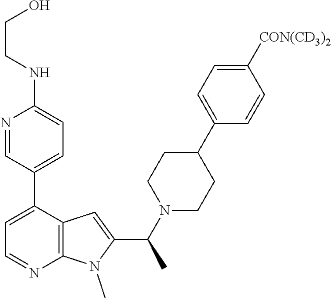

### Example 1af69. (S)-4-(1-(1-(4-(6-((2-hydroxyethyl)amino)pyridin-3-yl)-1-methyl-1H-pyrrolo[2,3-b]pyridin-2-yl)ethyl)piperidin-4-yl)-N,N-bis(methyl-d₃)benzamide

Int. 1AF75 (60 mg), 2-[(5-bromo-2-pyridyl)amino]ethanol (35 mg), BrettPhos Pd G4 (13 mg), and K₃PO₄ (87 mg) were degassed in dioxane/water (4:1, 2 mL) and stirred 1 h at 100° C. under MW conditions. The mixture was filtered. The OL (water/5% MeOH in DCM) was dried, concentrated, and HPLC-purified to give Example 1af69 (20 mg).

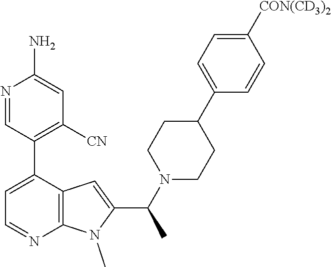

### Example 1af70. (S)-4-(1-(1-(4-(6-amino-4-cyanopyridin-3-yl)-1-methyl-1H-pyrrolo[2,3-b]pyridin-2-yl)ethyl)piperidin-4-yl)-N,N-bis(methyl-d₃)benzamide

Int. 1AF75 (60 mg), 2-amino-5-bromo-pyridine-4-carbonitrile (30 mg), BrettPhos Pd G4 (11 mg), and K₃PO₄ (94 mg) were degassed in dioxane/water (4:1, 2.5 mL) and stirred ON at 110° C. The mixture was filtered. The filtrate was partitioned between water and 5% MeOH in DCM. The OL was dried, concentrated, and HPLC-purified to give Example 1af70 (25 mg).

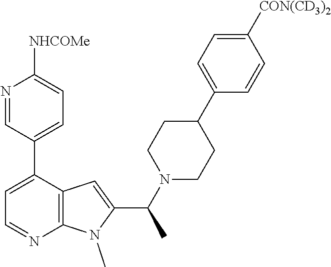

### Example 1af72. 4-((2R)-1-((4-(6-acetamidopyridin-3-yl)-1-methyl-1H-pyrrolo[2,3-b]pyridin-2-yl)methyl)-2-methylpiperidin-4-yl)-N,N-bis(methyl-d₃)benzamide

Int. 1AF75 (0.2 g), N-(5-bromo-1-oxido-pyridin-1-ium-2-yl)acetamide (0.12 g), PdDPPFCl₂-DCM (37 mg), and NaHCO₃ (0.11 g) were degassed in dioxane/water (4:1, 20 mL) and stirred ON at 100° C. The mixture was filtered, concentrated, and purified by FC (DCM/MeOH 9:1) and by HPLC to afford Example 1af72 (17 mg).

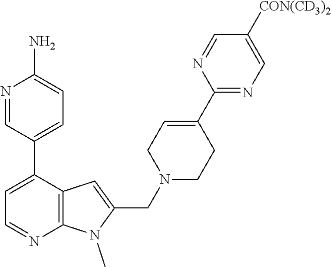

### Example 1af73. 2-(1-((4-(6-Aminopyridin-3-yl)-1-methyl-1H-pyrrolo[2,3-b]pyridin-2-yl)methyl)-1,2,3,6-tetrahydropyridin-4-yl)-N,N-bis(methyl-d₃)pyrimidine-5-carboxamide

Int. 1AF3 (2.0 g), (6-aminopyridin-3-yl)boronic acid (2.0 g), PdDPPFCl₂-DCM (0.30 g), and K₂CO₃ (3.0 g) were degassed in dioxane (30 mL) and stirred ON at 80° C. The OL (EtOAc/water) was dried, concentrated, and purified by HPLC to give Example 1af73 (1.5 g).

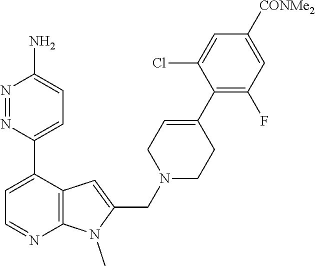

### Example 1af75. 4-(1-((4-(6-Aminopyridazin-3-yl)-1-methyl-1H-pyrrolo[2,3-b]pyridin-2-yl)methyl)-1,2,3,6-tetrahydropyridin-4-yl)-3-chloro-5-fluoro-N,N-dimethylbenzamide

Int. 1AF82 (0.18 g), tert-butyl (6-bromo-3-pyridazinyl)carbamate (98 mg), K₂CO₃ (0.13 g), and AdBrettPhos Pd G3 (6.6 mg) were degassed in dioxane (20 mL) and stirred at 90° C. for 2 days. The OL (water/MTBE) was dried and concentrated. The residue was stirred 0.5 h in THF (20 mL) and 4M HCl in dioxane (5 mL), concentrated, and HPLC-purified to give Example 1af75 (16 mg).

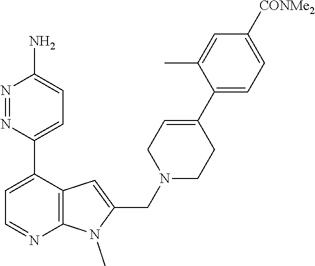

### Example 1af76. 4-(1-((4-(6-Aminopyridazin-3-yl)-1-methyl-1H-pyrrolo[2,3-b]pyridin-2-yl)methyl)-1,2,3,6-tetrahydropyridin-4-yl)-N,N,3-trimethylbenzamide

Int. 1AB5 (0.12 g), B₂Pin₂ (0.13 g), KOAc (0.1 g), PdDPPFCl₂-DCM (4 mg) were degassed in dioxane (10 mL) and stirred 48 h at 90° C. The OL (water/TMBE) was dried and concentrated. The residue, tert-butyl (6-bromo-3-pyridazinyl)carbamate (76 mg), K₂CO₃ (0.10 g), AdBrettPhos Pd G3 (5 mg) were degassed in dioxane (5 mL) and stirred 48 h at 90° C. The OL (water/MTBE) was dried and concentrated. The residue was stirred 0.5 h in THF (20 mL) and 4M HCl in dioxane (5 mL). The OL (THF/water) was dried, concentrated, and purified by HPLC to give Example 1af76 (11 mg).

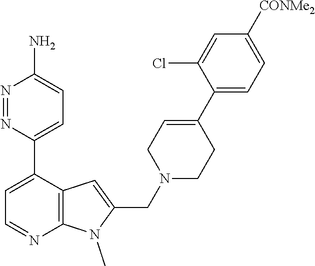

### Example 1af77. 4-(1-((4-(6-Aminopyridazin-3-yl)-1-methyl-1H-pyrrolo[2,3-b]pyridin-2-yl)methyl)-1,2,3,6-tetrahydropyridin-4-yl)-3-chloro-N,N-dimethylbenzamide

Int. 1AF8 (2 g), tert-butyl (6-bromo-3-pyridazinyl)carbamate (2 g), K₂CO₃ (3 g), and PdDPPFCl₂-DCM (0.3 g) were degassed in dioxane (30 mL) and stirred ON at 80° C. The OL (water/dioxane) was dried, concentrated, stirred ON in MeOH/4M HCl in dioxane (4:1, 50 mL), concentrated, and HPLC-purified to give Example 1af77 (0.41 g).

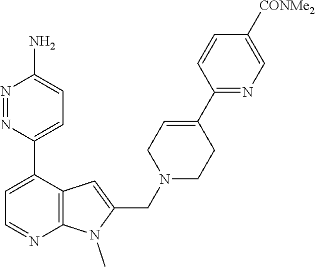

### Example 1af78. 1′-((4-(6-Aminopyridazin-3-yl)-1-methyl-1H-pyrrolo[2,3-b]pyridin-2-yl)methyl)-N,N-dimethyl-1′,2′,3′,6′-tetrahydro-[2,4′-bipyridine]-5-carboxamide

Int. 1AF12 (2.0 g), tert-butyl (6-bromo-3-pyridazinyl)carbamate (2.0 g), PdDPPFCl₂-DCM (0.3 g), and K₂CO₃ (3.0 g) were degassed in dioxane (30 mL) and stirred at 80° C. ON. The OL (EtOAc/water) was concentrated. 1.4 g of the residue was stirred in MeOH (40 mL) and 4M HCl in dioxane (5.8 mL) ON, concentrated, and HPLC-purified to give Example 1af78 (0.41 g).

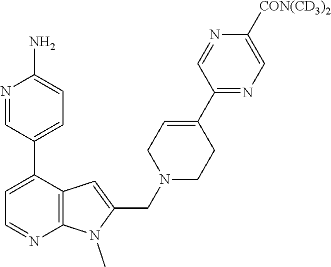

### Example 1af79. 5-(1-((4-(6-Aminopyridin-3-yl)-1-methyl-1H-pyrrolo[2,3-b]pyridin-2-yl)methyl)-1,2,3,6-tetrahydropyridin-4-yl)-N,N-bis(methyl-d3)pyrazine-2-carboxamide

(6-Aminopyridin-3-yl)boronic acid (2.0 g), Int. 1AF15 (2.0 g), K₂CO₃ (3.0 g), and PdDPPFCl₂-DCM (0.3 g) were degassed in dioxane (30 mL) and stirred ON at 80° C. The OL (EtOAc/water) was concentrated and purified by HPLC to give Example 1af79 (1.5 g).

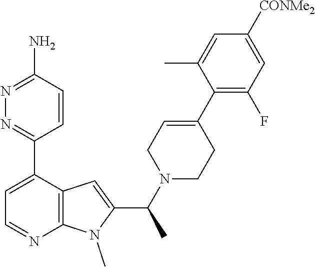

### Example 1af80. (S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-3-fluoro-N,N,5-trimethylbenzamide

Int. 1AF83 (0.15 g), 6-bromopyridazin-3-amine (40 mg), K₃PO₄ (0.16 g), and PdDPPFCl₂-DCM (16 mg) were degassed in dioxane/water (4:1, 5 mL) and stirred 1 h at 110° under MW conditions. The mixture was filtered, concentrated, and purified by FC (DCM/MeOH 91:9) to give Example 1af80 (50 mg). This material was mixed with 0.12 g prepared similarly on 0.5 g scale and precipitated from EtOAc/water to give Example 1af80 (0.11 g).

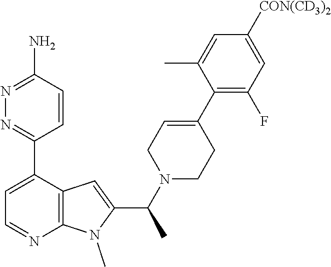

### Example 1af81. (S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-3-fluoro-5-methyl-N,N-bis(methyl-d₃)benzamide

Int. 1AF94 (0.30 g), 6-bromopyridazin-3-amine (0.11 g), K₃PO₄ (0.41 g), and PdDPPFCl₂-DCM (52 mg) were degassed in dioxane/water (5:1, 6 mL) and stirred 1 h at 120° C. under MW conditions. The mixture was concentrated and purified by FC (DCM/MeOH 9:1) to give Example 1af81 (0.20 g).

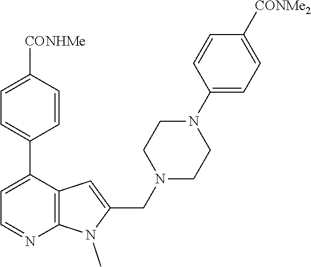

### Example 1j6. 4-[2-[[4-[4-(Dimethylcarbamoyl)phenyl]piperazin-1-yl]methyl]-1-methyl-pyrrolo[2,3-b]pyridin-4-yl]-N-methyl-benzamide

Int. 1J2 (21 mg), N,N-dimethyl-4-(piperazin-1-yl)benzamide (39 mg, HCl salt), and 4A MS were stirred 0.5 h in DCM (2 mL). STAB (46 mg) was added and stirring was continued ON. The mixture was filtered and HPLC-purified to give Example 1j6 (10 mM in DMSO (3.46 mL)).

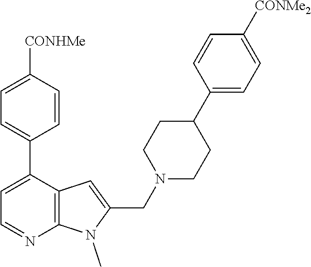

### Example 1j7. 4-[2-[[4-[4-(Dimethylcarbamoyl)phenyl]-1-piperidyl]methyl]-1-methyl-pyrrolo[2,3-b]pyridin-4-yl]-N-methyl-benzamide

Example 1j7 (10 mM in DMSO (3.47 mL)) was prepared similarly to Example 1j6 from Int. 1J2 (21 mg) and N,N-dimethyl-4-(piperidin-4-yl)benzamide (33 mg).

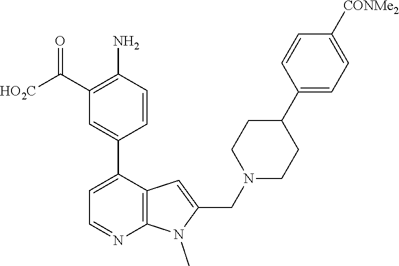

### Examples 1j13. 2-(2-Amino-5-(2-((4-(4-(dimethylcarbamoyl)phenyl)piperidin-1-yl)methyl)-1-methyl-1H-pyrrolo[2,3-b]pyridin-4-yl)phenyl)-2-oxoacetic acid

Int. 1AB4 (30 mg), 5-bromo-3-hydroxyindolin-2-one (11 mg), and PdDPPFCl₂-DCM (5 mg) were degassed in DMF/10% aq. Na₂CO₃ (5:1, 1.2 mL) and stirred ON at 100° C. The mixture was filtered and HPLC-purified to give Example 1j13 (1.4 mM in DMSO (0.50 mL)) [ring-opening occurred during work-up].

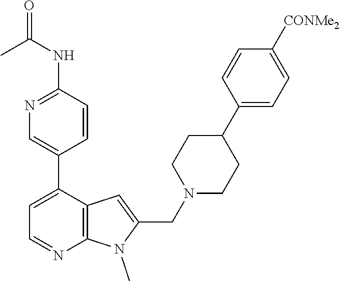

### Example 1k2. 4-[1-[[4-(6-Acetamido-3-pyridyl)-1-methyl-pyrrolo[2,3-b]pyridin-2-yl]methyl]-4-piperidyl]-N,N-dimethyl-benzamide

N-(5-(4,4,5,5-tetramethyl-1,3,2-dioxaborolan-2-yl)pyridin-2-yl)acetamide (17 mg), Int. 1AB1 (20 mg), K₂CO₃ (18 mg), and PdDPPFCl₂-DCM (4 mg) (18 mg) were degassed in DMF/water (5:1, 1.2 and stirred 0.5 h at 75° C. The mixture was filtered and purified by HPLC to give Example 1k2 (10 mM in DMSO (3.20 mL)).

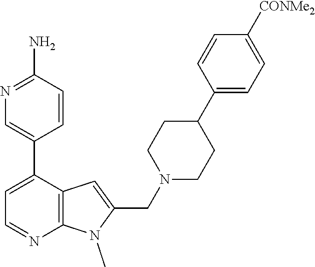

### Example 1k3. 4-[1-[[4-(6-Amino-3-pyridyl)-1-methyl-pyrrolo[2,3-b]pyridin-2-yl]methyl]-4-piperidyl]-N,N-dimethyl-benzamide

Example 1k3 (10 mM in DMSO (3.30 mL)) was prepared similarly to Example 1k2 from 5-(4,4,5,5-tetramethyl-1,3,2-dioxaborolan-2-yl)pyridin-2-amine (15 mg) and Int. 1AB1 (20 mg).

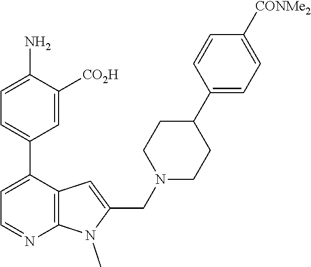

### Example 1k6. 2-Amino-5-[2-[[4-[4-(dimethylcarbamoyl)phenyl]-1-piperidyl]methyl]-1-methyl-pyrrolo[2,3-b]pyridin-4-yl]benzoic acid

Int. 1AB4 (25 mg), 2-amino-5-bromobenzoic acid (21 mg), Na₂CO₃ (16 mg), and PdDPPFCl₂-DCM (4 mg) were degassed in DMF/water (4:1, 1.3 mL) and stirred ON at 80° C. The mixture was filtered and purified by HPLC to give Example 1k6 (2 mM in DMSO (0.22 mL)).

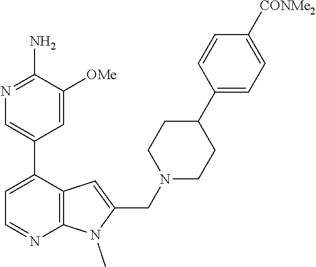

### Example 1k7. 4-(1-((4-(6-Amino-5-methoxypyridin-3-yl)-1-methyl-1H-pyrrolo[2,3-b]pyridin-2-yl)methyl)piperidin-4-yl)-N,N-dimethylbenzamide

Int. 1AB4 (10 mg), 5-bromo-6-methoxypyridin-2-amine (8 mg), Na₂CO₃ (6 mg), and PdDPPFCl₂-DCM (2 mg) were degassed in DMF/water (3.8:1, 0.6 mL) and stirred 0.5 h at 80° C. The mixture was filtered and purified by HPLC to give Example 1k7 (2.84 mM in DMSO (0.70 mL)).

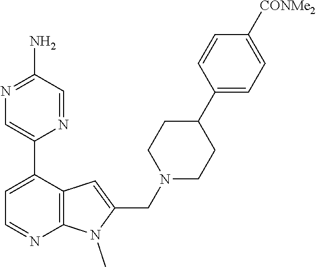

### Example 1k8. 4-[1-[[4-(5-Aminopyrazin-2-yl)-1-methyl-pyrrolo[2,3-b]pyridin-2-yl]methyl]-4-piperidyl]-N,N-dimethyl-benzamide

Example 1k8 (5.77 mM in DMSO (0.70 mL)) was prepared similarly to Example 1k7 from 5-bromopyrazin-2-amine (7 mg).

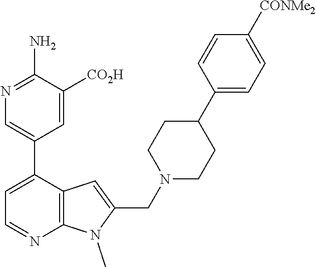

### Example 1k9. 2-Amino-5-[2-[[4-[4-(dimethylcarbamoyl)phenyl]-1-piperidyl]methyl]-1-methyl-pyrrolo[2,3-b]pyridin-4-yl]pyridine-3-carboxylic acid

2-Amino-5-bromonicotinic acid (9 mg) and PdDPPFCl₂-DCM (2 mg) were added to a vial and degassed. Int. 1AB4 (0.5 mL of a degassed solution of 0.25 g in DMF (12.5 mL)) and Na₂CO₃ (0.13 mL of a degassed solution of 0.16 g in water (3.12 mL)) were added. The mixture was stirred at 80° C. ON and HPLC-purified to give Example 1k9 (1.45 mM in DMSO (0.80 mL)).

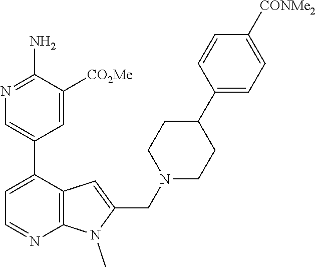

### Example 1k10. Methyl 2-amino-5-[2-[[4-[4-(dimethylcarbamoyl)phenyl]-1-piperidyl]methyl]-1-methyl-pyrrolo[2,3-b]pyridin-4-yl]pyridine-3-carboxylate

Example 1k10 (10 mM in DMSO (1.36 mL)) was prepared similarly to Example 1k9 from Int. 1AB4 (25 mg) and methyl 2-amino-5-bromo-pyridine-3-carboxylate (23 mg).

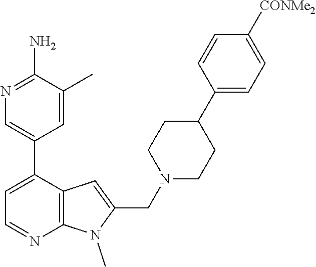

### Example 1k11. 4-[1-[[4-(6-Amino-5-methyl-3-pyridyl)-1-methyl-pyrrolo[2,3-b]pyridin-2-yl]methyl]-4-piperidyl]-N,N-dimethyl-benzamide

Example 1k11 (2.0 mM in DMSO (0.70 mL)) was prepared similarly to Example 1k7 from 5-bromo-3-methylpyridin-2-amine (7 mg).

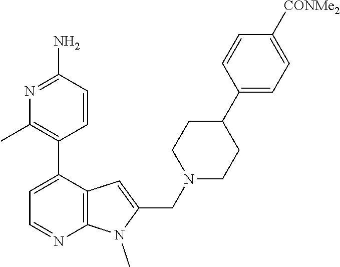

### Example 1k12. 4-[1-[[4-(6-Amino-2-methyl-3-pyridyl)-1-methyl-pyrrolo[2,3-b]pyridin-2-yl]methyl]-4-piperidyl]-N,N-dimethyl-benzamide

Example 1k12 (6.3 mM in DMSO (0.70 mL)) was prepared similarly to Example 1k7 from 4-bromo-3-methylpyridin-2-amine (7 mg).

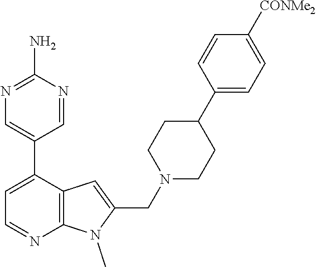

### Example 1k13. 4-[1-[[4-(2-Aminopyrimidin-5-yl)-1-methyl-pyrrolo[2,3-b]pyridin-2-yl]methyl]-4-piperidyl]-N,N-dimethyl-benzamide

Example 1k13 (8.7 mM in DMSO (0.70 mL)) was prepared similarly to Example 1k7 from 5-bromopyrimidin-2-amine (7 mg).

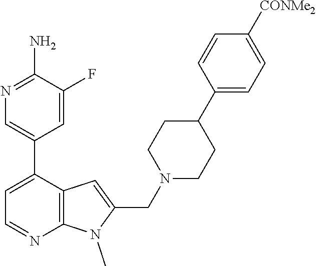

### Example 1k14. 4-[1-[[4-(6-Amino-5-fluoro-3-pyridyl)-1-methyl-pyrrolo[2,3-b]pyridin-2-yl]methyl]-4-piperidyl]-N,N-dimethyl-benzamide

Example 1k14 (7.9 mM in DMSO (0.70 mL)) was prepared similarly to Example 1k7 from 5-bromo-3-fluoro-2-amine (7 mg).

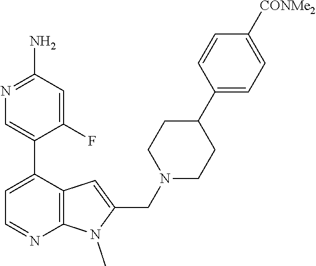

### Example 1k15. 4-[1-[[4-(6-Amino-4-fluoro-3-pyridyl)-1-methyl-pyrrolo[2,3-b]pyridin-2-yl]methyl]-4-piperidyl]-N,N-dimethyl-benzamide

Example 1k15 (6.0 mM in DMSO (0.70 mL)) was prepared similarly to Example 1k7 from 5-bromo-4-fluoro-2-amine (7 mg).

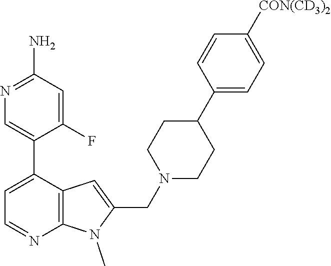

### Example 1k16. 4-[1-[[4-(6-Amino-4-fluoro-3-pyridyl)-1-methyl-pyrrolo[2,3-b]pyridin-2-yl]methyl]-4-piperidyl]-N,N-bis(trideuteriomethyl)benzamide

5-Bromo-4-fluoropyridin-2-amine (7 mg), PdDPPFCl₂-DCM (3 mg), Int. 1AB4′ (0.5 mL of a solution of 0.18 g in DMF (3 mL)) and Na₂CO₃ (0.125 mL of a solution of 0.14 g in water (0.75 mL)) were degassed and stirred at 120° C. ON. The mixture was filtered and HPLC-purified to give Example 1k16 (2.2 mM in DMSO (0.70 mL)).

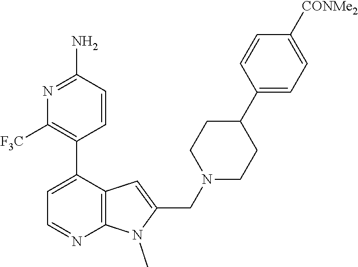

### Example 1k17. 4-[1-[[4-[6-Amino-2-(trifluoromethyl)-3-pyridyl]-1-methyl-pyrrolo[2,3-b]pyridin-2-yl]methyl]-4-piperidyl]-N,N-dimethyl-benzamide

Example 1k17 (8.9 mM in DMSO (0.70 mL)) was prepared similarly to Example 1k7 from 5-bromo-6-(trifluoromethyl)pyridin-2-amine (9 mg).

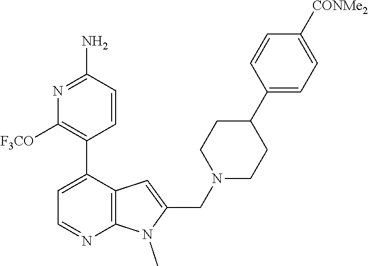

### Example 1k18. 4-[1-[[4-[6-Amino-2-(trifluoromethoxy)-3-pyridyl]-1-methyl-pyrrolo[2,3-b]pyridin-2-yl]methyl]-4-piperidyl]-N,N-dimethyl-benzamide

Example 1k18 (3.8 mM in DMSO (0.80 mL)) was prepared similarly to Example 1k7 from 5-bromo-6-(trifluoromethoxy)pyridin-2-amine (9 mg).

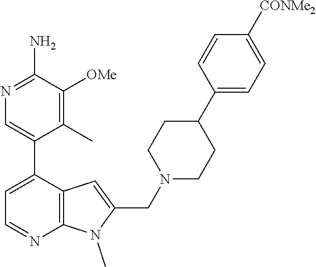

### Example 1k19. 4-[1-[[4-(6-Amino-5-methoxy-4-methyl-3-pyridyl)-1-methyl-pyrrolo[2,3-b]pyridin-2-yl]methyl]-4-piperidyl]-N,N-dimethyl-benzamide

Example 1k19 (4.2 mM in DMSO (0.80 mL)) was prepared similarly to Example 1k7 from 5-bromo-3-methoxy-4-methylpyridin-2-amine (9 mg).

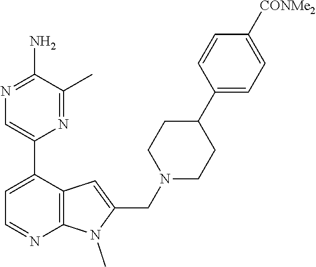

### Example 1k20. 4-[1-[[4-(5-Amino-6-methyl-pyrazin-2-yl)-1-methyl-pyrrolo[2,3-b]pyridin-2-yl]methyl]-4-piperidyl]-N,N-dimethyl-benzamide

Example 1k20 (5.7 mM in DMSO (0.80 mL)) was prepared similarly to Example 1k7 from 5-bromo-3-methylpyrazin-2-amine (8 mg).

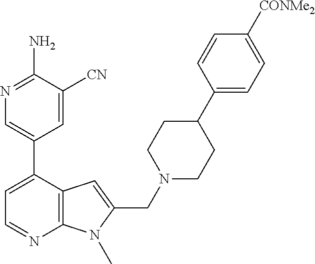

### Example 1k21. 4-[1-[[4-(6-Amino-5-cyano-3-pyridyl)-1-methyl-pyrrolo[2,3-b]pyridin-2-yl]methyl]-4-piperidyl]-N,N-dimethyl-benzamide

Example 1k21 (4.8 mM in DMSO (0.80 mL)) was prepared similarly to Example 1k7 from 2-amino-5-bromonicotinonitrile (9 mg).

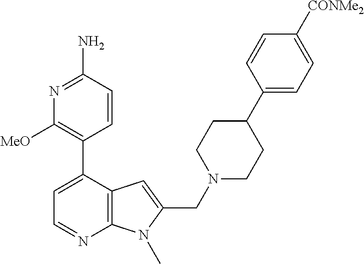

### Example 1k22. 4-[1-[[4-(6-Amino-2-methoxy-3-pyridyl)-1-methyl-pyrrolo[2,3-b]pyridin-2-yl]methyl]-4-piperidyl]-N,N-dimethyl-benzamide

Example 1k22 (5.2 mM in DMSO (0.80 mL)) was prepared similarly to Example 1k7 from 5-bromo-6-methoxypyridin-2-amine (9 mg).

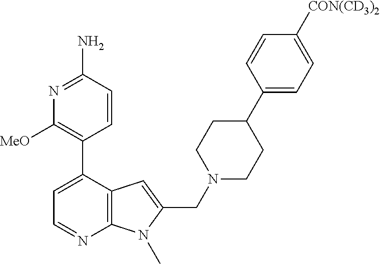

### Example 1k23. 4-[1-[[4-(6-Amino-2-methoxy-3-pyridyl)-1-methyl-pyrrolo[2,3-b]pyridin-2-yl]methyl]-4-piperidyl]-N,N-bis(trideuteriomethyl)benzamide

Example 1k23 (5.6 mM in DMSO (0.80 mL)) was prepared similarly to Example 1k16 from 5-bromo-6-methoxypyridin-2-amine (8 mg).

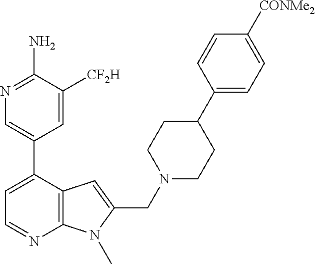

### Example 1k24. 4-[1-[[4-[6-Amino-5-(difluoromethyl)-3-pyridyl]-1-methyl-pyrrolo[2,3-b]pyridin-2-yl]methyl]-4-piperidyl]-N,N-dimethyl-benzamide

Example 1k24 (5.5 mM in DMSO (0.80 mL)) was prepared similarly to Example 1k7 from 5-bromo-3-(difluoromethyl)pyridin-2-amine (12 mg).

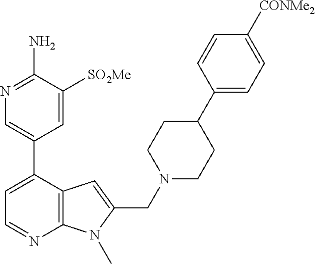

### Example 1k25. 4-[1-[[4-(6-Amino-5-methylsulfonyl-3-pyridyl)-1-methyl-pyrrolo[2,3-b]pyridin-2-yl]methyl]-4-piperidyl]-N,N-dimethyl-benzamide

Example 1k25 (3.3 mM in DMSO (0.80 mL)) was prepared similarly to Example 1k7 from 5-bromo-3-(methylsulfonyl)pyridin-2-amine (13 mg).

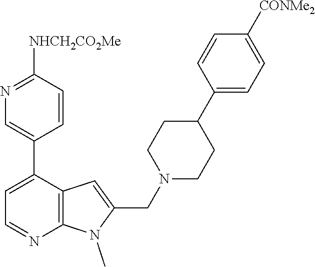

### Example 1k26. Methyl 2-[[5-[2-[[4-[4-(dimethylcarbamoyl)phenyl]-1-piperidyl]methyl]-1-methyl-pyrrolo[2,3-b]pyridin-4-yl]-2-pyridyl]amino]acetate

Example 1k26 (1.6 mM in DMSO (0.80 mL)) was prepared similarly to Example 1k7 from methyl (5-bromopyridin-2-yl)carbamate (11 mg).

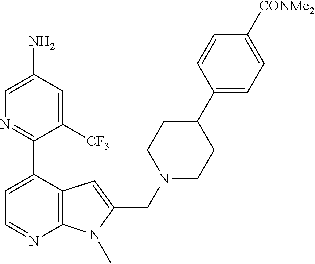

### Example 1k27. 4-[1-[[4-[6-Amino-4-(trifluoromethyl)-3-pyridyl]-1-methyl-pyrrolo[2,3-b]pyridin-2-yl]methyl]-4-piperidyl]-N,N-dimethyl-benzamide

Example 1k27 (4.1 mM in DMSO (0.80 mL)) was prepared similarly to Example 1k7 from 5-bromo-4-(trifluoromethyl)pyridin-2-amine (11 mg).

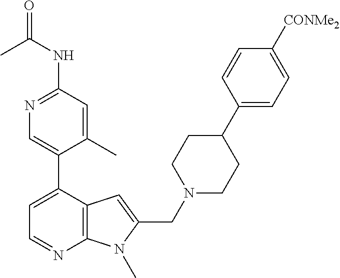

### Example 1k28. 4-[1-[[4-(6-Acetamido-4-methyl-3-pyridyl)-1-methyl-pyrrolo[2,3-b]pyridin-2-yl]methyl]-4-piperidyl]-N,N-dimethyl-benzamide

Example 1k28 (6.0 mM in DMSO (0.80 mL)) was prepared similarly to Example 1k7 from N-(5-bromo-4-methylpyridin-2-yl)acetamide (12 mg).

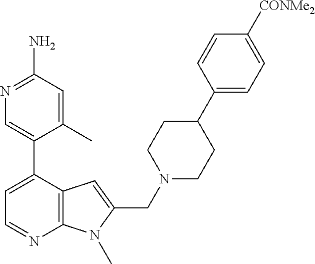

### Example 1k30. 4-[1-[[4-(6-Amino-4-methyl-3-pyridyl)-1-methyl-pyrrolo[2,3-b]pyridin-2-yl]methyl]-4-piperidyl]-N,N-dimethyl-benzamide

Example 1k30 (4.0 mM in DMSO (0.80 mL)) was prepared similarly to Example 1k7 from 5-bromo-4-methylpyridin-2-amine (14 mg).

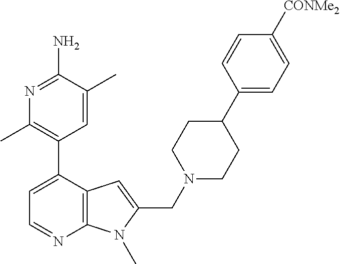

### Example 1k31. 4-[1-[[4-(6-Amino-2,5-dimethyl-3-pyridyl)-1-methyl-pyrrolo[2,3-b]pyridin-2-yl]methyl]-4-piperidyl]-N,N-dimethyl-benzamide

Example 1k31 (4.3 mM in DMSO (0.80 mL)) was prepared similarly to Example 1k7 from 5-bromo-3,6-dimethylpyridin-2-amine (14 mg).

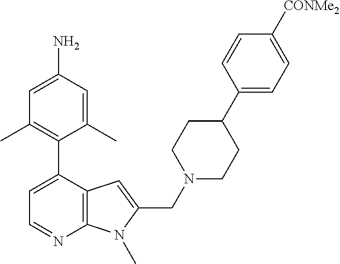

### Example 1k32. 4-[1-[[4-(4-Amino-2,6-dimethyl-phenyl)-1-methyl-pyrrolo[2,3-b]pyridin-2-yl]methyl]-4-piperidyl]-N,N-dimethyl-benzamide

Example 1k32 (4.6 mM in DMSO (0.80 mL)) was prepared similarly to Example 1k7 from 4-bromo-3,5-dimethylaniline (14 mg).

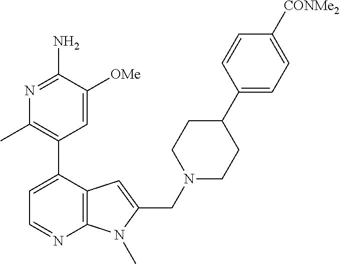

### Example 1k33. 4-[1-[[4-(6-Amino-5-methoxy-2-methyl-3-pyridyl)-1-methyl-pyrrolo[2,3-b]pyridin-2-yl]methyl]-4-piperidyl]-N,N-dimethyl-benzamide

Example 1k33 (4.3 mM in DMSO (0.80 mL)) was prepared similarly to Example 1k7 from 5-bromo-3-methoxy-6-methylpyridin-2-amine (14 mg).

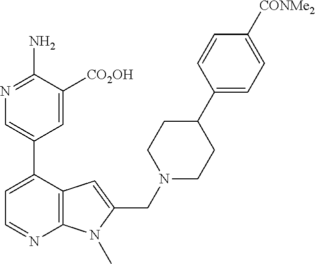

### Example 1k34. 4-[1-[[4-[6-Amino-5-(hydroxymethyl)-3-pyridyl]-1-methyl-pyrrolo[2,3-b]pyridin-2-yl]methyl]-4-piperidyl]-N,N-dimethyl-benzamide

Example 1k34 (3.9 mM in DMSO (0.80 mL)) was prepared similarly to Example 1k7 from (2-amino-5-bromopyridin-3-yl)methanol (14 mg).

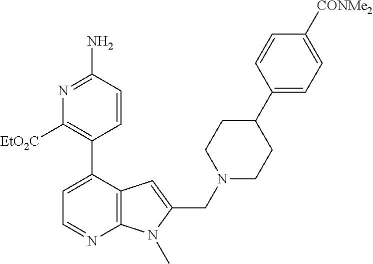

### Example 1k35. Ethyl 6-amino-3-[2-[[4-[4-(dimethylcarbamoyl)phenyl]-1-piperidyl]methyl]-1-methyl-pyrrolo[2,3-b]pyridin-4-yl]pyridine-2-carboxylate

Example 1k35 (7.0 mM in DMSO (0.80 mL)) was prepared similarly to Example 1k7 from ethyl 6-amino-3-bromopicolinate (15 mg).

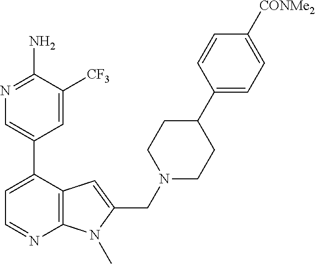

### Example 1k36. 4-[1-[[4-[6-Amino-5-(trifluoromethyl)-3-pyridyl]-1-methyl-pyrrolo[2,3-b]pyridin-2-yl]methyl]-4-piperidyl]-N,N-dimethyl-benzamide

Example 1k36 (6.2 mM in DMSO (0.80 mL)) was prepared similarly to Example 1k7 from 5-bromo-3-(trifluoromethyl)pyridin-2-amine (15 mg).

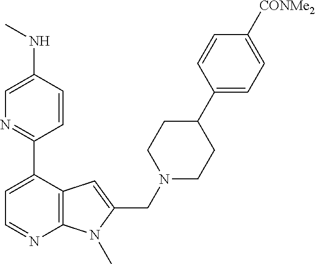

### Example 1k37. N,N-Dimethyl-4-[1-[[1-methyl-4-[6-(methylamino)-3-pyridyl]pyrrolo[2,3-b]pyridin-2-yl]methyl]-4-piperidyl]benzamide

Example 1k37 (5.4 mM in DMSO (0.80 mL)) was prepared similarly to Example 1k7 from 5-bromo-N-methylpyridin-2-amine (9 mg).

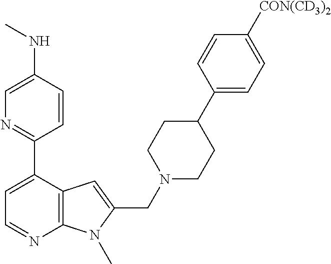

### Example 1k38. 4-[1-[[1-Methyl-4-[6-(methylamino)-3-pyridyl]pyrrolo[2,3-b]pyridin-2-yl]methyl]-4-piperidyl]-N,N-bis(trideuteriomethyl)benzamide

Example 1k38 (4.6 mM in DMSO (0.80 mL)) was prepared similarly to Example 1k16 from 5-bromo-N-methylpyridin-2-amine (7 mg).

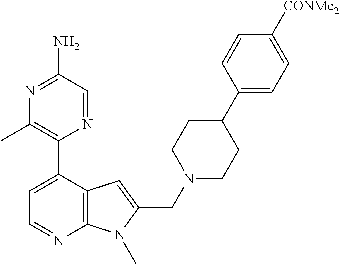

### Example 1k39. 4-[1-[[4-(5-Amino-3-methyl-pyrazin-2-yl)-1-methyl-pyrrolo[2,3-b]pyridin-2-yl]methyl]-4-piperidyl]-N,N-dimethyl-benzamide

Example 1k39 (5.2 mM in DMSO (0.80 mL)) was prepared similarly to Example 1k7 from 5-bromo-6-methylpyrazin-2-amine (9 mg).

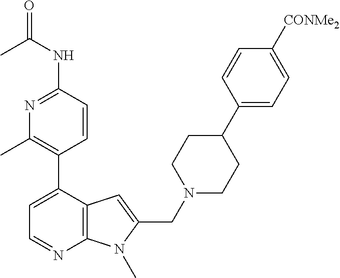

### Example 1k40. 4-[1-[[4-(6-Acetamido-2-methyl-3-pyridyl)-1-methyl-pyrrolo[2,3-b]pyridin-2-yl]methyl]-4-piperidyl]-N,N-dimethyl-benzamide

Example 1k40 (7.6 mM in DMSO (0.80 mL)) was prepared similarly to Example 1k7 from N-(5-bromo-6-methylpyridin-2-yl)acetamide (13 mg).

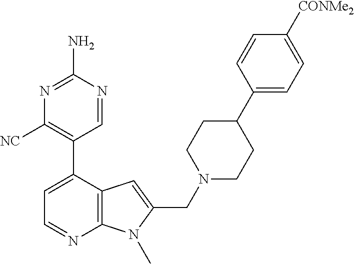

### Example 1k41. 4-[1-[[4-(2-Amino-4-cyano-pyrimidin-5-yl)-1-methyl-pyrrolo[2,3-b]pyridin-2-yl]methyl]-4-piperidyl]-N,N-dimethyl-benzamide

Example 1k41 (7.8 mM in DMSO (0.80 mL)) was prepared similarly to Example 1k7 from 2-amino-5-bromopyrimidine-4-carbonitrile (14 mg).

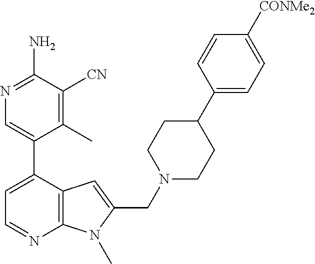

### Example 1k42. 4-[1-[[4-(6-Amino-5-cyano-4-methyl-3-pyridyl)-1-methyl-pyrrolo[2,3-b]pyridin-2-yl]methyl]-4-piperidyl]-N,N-dimethyl-benzamide

Example 1k42 (7.4 mM in DMSO (0.80 mL)) was prepared similarly to Example 1k7 from 2-amino-5-bromo-4-methylnicotinonitrile (15 mg).

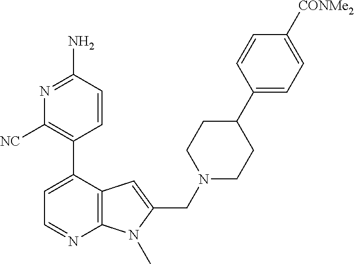

### Example 1k43. 4-[1-[[4-(6-Amino-2-cyano-3-pyridyl)-1-methyl-pyrrolo[2,3-b]pyridin-2-yl]methyl]-4-piperidyl]-N,N-dimethyl-benzamide

Example 1k43 (9.1 mM in DMSO (0.80 mL)) was prepared similarly to Example 1k7 from 6-amino-3-bromopicolinonitrile (12 mg).

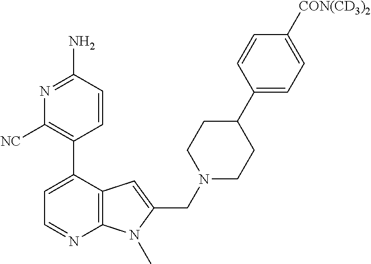

### Example 1k44. 4-[1-[[4-(6-Amino-2-methoxy-3-pyridyl)-1-methyl-pyrrolo[2,3-b]pyridin-2-yl]methyl]-4-piperidyl]-N,N-bis(trideuteriomethyl)benzamide

Example 1k44 (3.6 mM in DMSO (0.80 mL)) was prepared similarly to Example 1k16 from 6-amino-3-bromopicolinonitrile (9 mg).

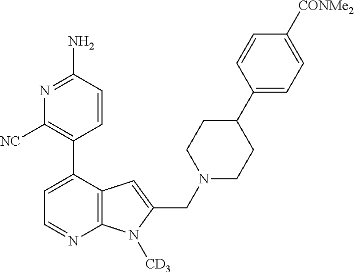

### Example 1k45. 4-[1-[[4-(6-Amino-2-cyano-3-pyridyl)-1-(trideuteriomethyl)pyrrolo[2,3-b]pyridin-2-yl]methyl]-4-piperidyl]-N,N-dimethyl-benzamide

Int. 1K1 (39 mg), B₂Pin₂ (43 mg), KOAc (25 mg), and PdDPPFCl₂-DCM (9 mg) were degassed in DMSO (2.5 mL) and stirred ON at 100° C. The OL (water/DCM) was dried and concentrated. The residue, 6-amino-3-bromo-picolinonitrile (11 mg), K₂CO₃ (23 mg), and PdDPPFCl₂-DCM (5 mg) were degassed in DMF/water (5:1, 1.8 mL) and stirred 1 h at 100° C., filtered, and HPLC-purified to give Example 1k45 (3.1 mM in DMSO (0.80 mL)).

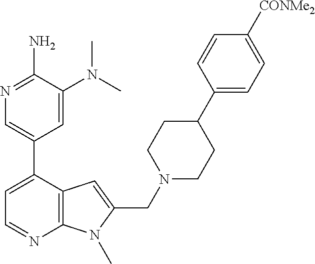

### Example 1k47. 4-[1-[[4-[6-Amino-5-(dimethylamino)-3-pyridyl]-1-methyl-pyrrolo[2,3-b]pyridin-2-yl]methyl]-4-piperidyl]-N,N-dimethyl-benzamide

Example 1k47 (6.6 mM in DMSO (0.80 mL)) was prepared similarly to Example 1k7 from 5-bromo-N3,N3-dimethylpyridine-2,3-diamine (13 mg).

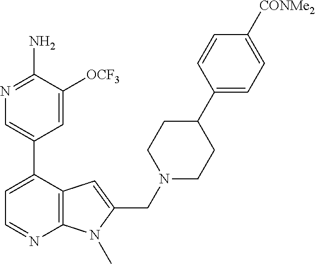

### Example 1k48. 4-[1-[[4-[6-Amino-5-(trifluoromethoxy)-3-pyridyl]-1-methyl-pyrrolo[2,3-b]pyridin-2-yl]methyl]-4-piperidyl]-N,N-dimethyl-benzamide

Example 1k48 (6.1 mM in DMSO (0.80 mL)) was prepared similarly to Example 1k7 from 5-bromo-3-(trifluoromethoxy)pyridin-2-amine (16 mg).

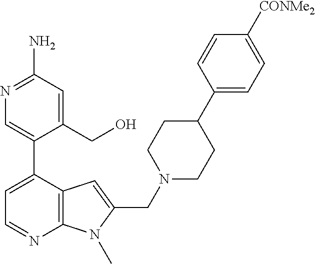

### Example 1k49. 4-[1-[[4-[6-Amino-4-(hydroxymethyl)-3-pyridyl]-1-methyl-pyrrolo[2,3-b]pyridin-2-yl]methyl]-4-piperidyl]-N,N-dimethyl-benzamide

Example 1k49 (4.2 mM in DMSO (0.80 mL)) was prepared similarly to Example 1k7 from (2-amino-5-bromopyridin-4-yl)methanol (13 mg).

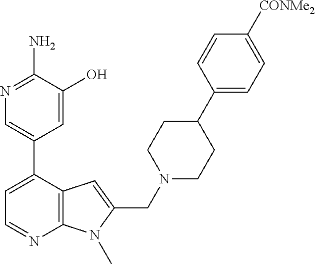

### Example 1k50. 4-[1-[[4-(6-Amino-5-hydroxy-3-pyridyl)-1-methyl-pyrrolo[2,3-b]pyridin-2-yl]methyl]-4-piperidyl]-N,N-dimethyl-benzamide

Example 1k50 (2.6 mM in DMSO (0.80 mL)) was prepared similarly to Example 1k7 from 2-amino-5-bromopyridin-3-ol (13 mg).

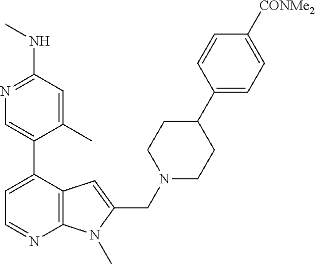

### Example 1k51. N,N-Dimethyl-4-[1-[[1-methyl-4-[4-methyl-6-(methylamino)-3-pyridyl]pyrrolo[2,3-b]pyridin-2-yl]methyl]-4-piperidyl]benzamide

Example 1k51 (8.6 mM in DMSO (0.80 mL)) was prepared similarly to Example 1k7 from 5-bromo-N,4-dimethylpyridin-2-amine (14 mg).

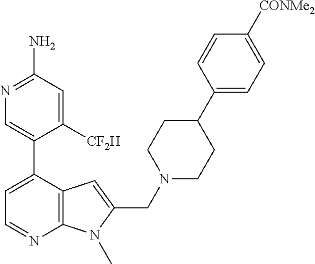

### Example 1k52. 4-[1-[[4-[6-Amino-4-(difluoromethyl)-3-pyridyl]-1-methyl-pyrrolo[2,3-b]pyridin-2-yl]methyl]-4-piperidyl]-N,N-dimethyl-benzamide

Example 1k52 (9.2 mM in DMSO (0.80 mL)) was prepared similarly to Example 1k7 from 5-bromo-4-(difluoromethyl)pyridin-2-amine (17 mg).

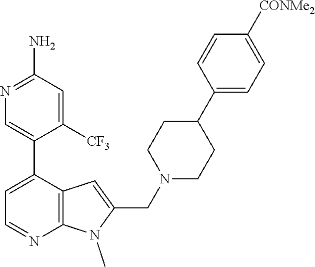

### Example 1k53. 4-[1-[[4-(6-Amino-4-cyano-3-pyridyl)-1-methyl-pyrrolo[2,3-b]pyridin-2-yl]methyl]-4-piperidyl]-N,N-dimethyl-benzamide

Example 1k53 (9.7 mM in DMSO (0.80 mL)) was prepared similarly to Example 1k7 from 2-amino-5-bromoisonicotinonitrile (13 mg).

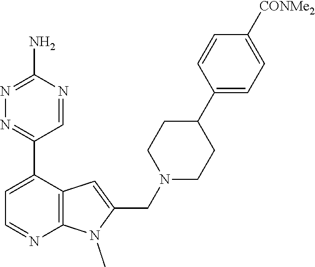

### Example 1k54. 4-[1-[[4-(3-Amino-1,2,4-triazin-6-yl)-1-methyl-pyrrolo[2,3-b]pyridin-2-yl]methyl]-4-piperidyl]-N,N-dimethyl-benzamide

Example 1k54 (10.0 mM in DMSO (1.1 mL)) was prepared similarly to Example 1k7 from 6-bromo-1,2,4-triazin-3-amine (9 mg).

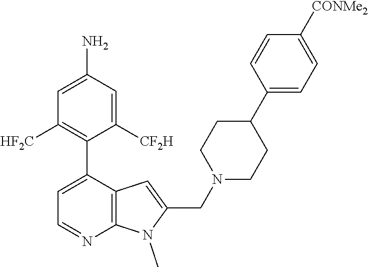

### Example 1k55. 4-[1-[[4-[4-Amino-2,6-bis(difluoromethyl)phenyl]-1-methyl-pyrrolo[2,3-b]pyridin-2-yl]methyl]-4-piperidyl]-N,N-dimethyl-benzamide

Example 1k55 (2.6 mM in DMSO (0.80 mL)) was prepared similarly to Example 1k7 from 4-bromo-3,5-bis(difluoromethyl)aniline (21 mg).

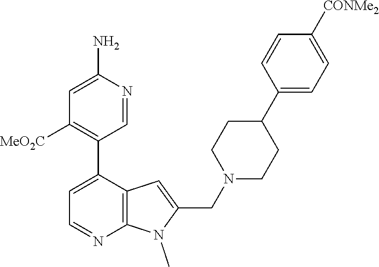

### Example 1k56. Methyl 2-amino-5-[2-[[4-[4-(dimethylcarbamoyl)phenyl]-1-piperidyl]methyl]-1-methyl-pyrrolo[2,3-b]pyridin-4-yl]pyridine-4-carboxylate

Example 1k56 (10.0 mM in DMSO (0.99 mL)) was prepared similarly to Example 1k7 from methyl 2-amino-5-bromoisonicotinate (18 mg).

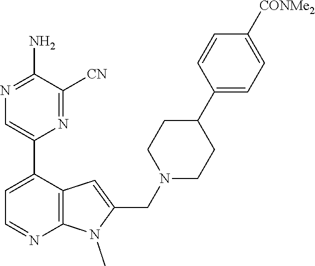

### Example 1k57. 4-[1-[[4-(5-Amino-6-cyano-pyrazin-2-yl)-1-methyl-pyrrolo[2,3-b]pyridin-2-yl]methyl]-4-piperidyl]-N,N-dimethyl-benzamide

Example 1k57 (5.6 mM in DMSO (0.80 mL)) was prepared similarly to Example 1k7 from 3-amino-6-bromopyrazine-2-carbonitrile (18 mg).

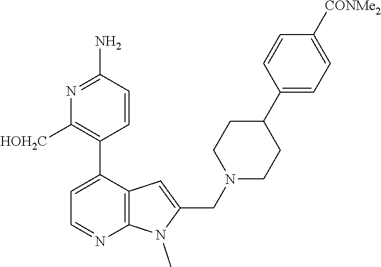

### Example 1k58. 4-[1-[[4-[6-Amino-2-(hydroxymethyl)-3-pyridyl]-1-methyl-pyrrolo[2,3-b]pyridin-2-yl]methyl]-4-piperidyl]-N,N-dimethyl-benzamide

Example 1k58 (9.5 mM in DMSO (0.80 mL)) was prepared similarly to Example 1k7 from (6-amino-3-bromopyridin-2-yl)methanol (18 mg).

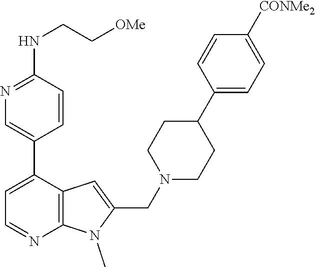

### Example 1k59. 4-[1-[[4-[6-(2-Methoxyethylamino)-3-pyridyl]-1-methyl-pyrrolo[2,3-b]pyridin-2-yl]methyl]-4-piperidyl]-N,N-dimethyl-benzamide

Example 1k59 (10.0 mM in DMSO (0.45 mL)) was prepared similarly to Example 1k7 from 5-bromo-N-(2-methoxyethyl)pyridin-2-amine (20 mg).

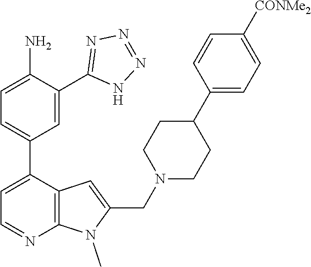

### Example 1k60. 4-[1-[[4-[4-Amino-3-(2H-tetrazol-5-yl)phenyl]-1-methyl-pyrrolo[2,3-b]pyridin-2-yl]methyl]-4-piperidyl]-N,N-dimethyl-benzamide

Example 1k80 (5 mg), NaN₃ (86 mg), and Et₃N (2 mg, HCl salt) were stirred in toluene (1 mL) ON at 80° C. and purified by HPLC to give Example 1k60 (4.3 mM in DMSO (0.44 mL)).

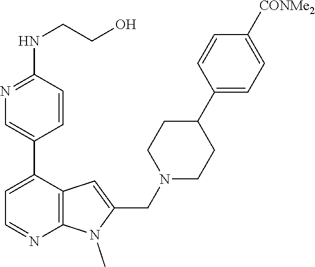

### Example 1k61. 4-[1-[[4-[6-(2-Hydroxyethylamino)-3-pyridyl]-1-methyl-pyrrolo[2,3-b]pyridin-2-yl]methyl]-4-piperidyl]-N,N-dimethyl-benzamide

Example 1k61 (7.99 mM in 0.5 DMSO) was prepared similarly to Example 1k9 from Int. 1AB4 (17 mg) and 2-[(5-bromo-2-pyridyl)amino]ethanol (18 mg).

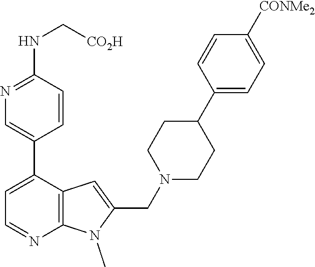

### Example 1k63. 2-[[5-[2-[[4-[4-(Dimethylcarbamoyl)phenyl]-1-piperidyl]methyl]-1-methyl-pyrrolo[2,3-b]pyridin-4-yl]-2-pyridyl]amino]acetic acid

Example 1k26 (9 mg) was stirred 0.5 h in MeOH (1 mL) and 2M aq. NaOH (25 μL). The mixture was purified by HPLC to give Example 1k63 (4.3 mg).

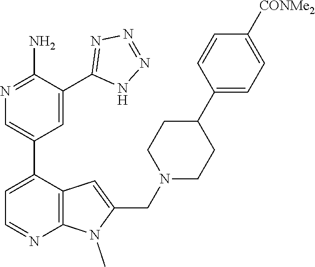

### Example 1k64. 4-[1-[[4-[6-Amino-5-(2H-tetrazol-5-yl)-3-pyridyl]-1-methyl-pyrrolo[2,3-b]pyridin-2-yl]methyl]-4-piperidyl]-N,N-dimethyl-benzamide

Example 1k21 (5 mg), NaN₃ (86 mg), and Et₃N (2 mg, HCl salt) were stirred ON in DMSO/touene (2:1, 1.5 mL) at 90° C. The mixture was purified by HPLC to give Example 1k64 (1.5 mM in DMSO (0.46 mL)).

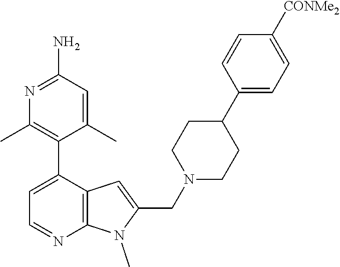

### Example 1k65. 4-[1-[[4-(6-Amino-2,4-dimethyl-3-pyridyl)-1-methyl-pyrrolo[2,3-b]pyridin-2-yl]methyl]-4-piperidyl]-N,N-dimethyl-benzamide

5-Bromo-4,6-dimethyl-pyridin-2-amine (50 mg), Int. 1AB4 (0.15 g), PdDPPFCl₂-DCM (20 mg), and NaHCO₃ (63 mg) were degassed inn dioxane (2.5 mL) and stirred 1 h at 135° C. under MW irradiation. The mixture was filtered, concentrated, and purified by FC (DCM/MeOH 9:1) and by HPLC to give Example 1k65 (41 mg).

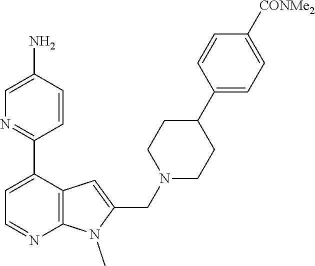

### Example 1k66. 4-(1-((4-(5-Aminopyridin-2-yl)-1-methyl-1H-pyrrolo[2,3-b]pyridin-2-yl)methyl)piperidin-4-yl)-N,N-dimethylbenzamide

Example 1k66 (0.31 g) was prepared similarly to Example 1ad1 from 6-bromopyridin-3-amine (0.26 g).

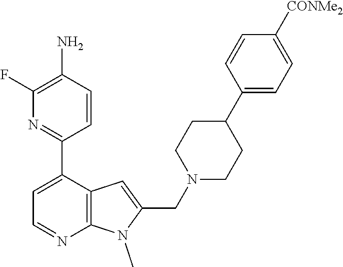

### Example 1k67. 4-(1-((4-(5-Amino-6-fluoropyridin-2-yl)-1-methyl-1H-pyrrolo[2,3-b]pyridin-2-yl)methyl)piperidin-4-yl)-N,N-dimethylbenzamide

Example 1k67 (19 mg) was prepared similarly to Example 1ad1 from 6-bromo-2-fluoropyridin-3-amine (11 mg).

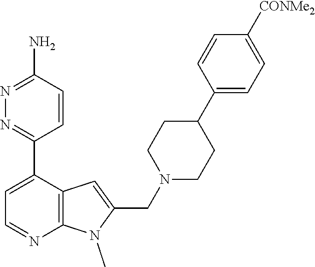

### Example 1k68. 4-[1-[[4-(6-Aminopyridazin-3-yl)-1-methyl-pyrrolo[2,3-b]pyridin-2-yl]methyl]-4-piperidyl]-N,N-dimethyl-benzamide

Example 1k68 (4.5 mM in DMSO (0.70 mL)) was prepared similarly to Example 1k9 from Int. 1AB4 (10 mg) and 6-bromopyridazin-3-amine (5 mg).

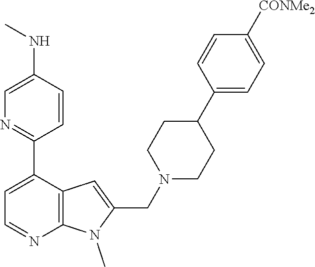

### Example 1k69. N,N-Dimethyl-4-(1-((1-methyl-4-(5-(methylamino)pyridin-2-yl)-1H-pyrrolo[2,3-b]pyridin-2-yl)methyl)piperidin-4-yl)benzamide

Example 1k69 (19 mg) was prepared similarly to Example 1ad1 from 6-bromo-N-methylpyridin-3-amine (11 mg).

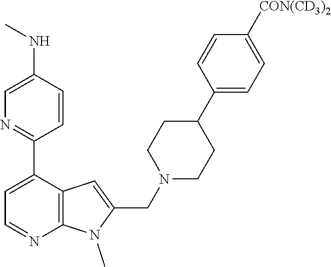

### Example 1k70. 4-[1-[[1-methyl-4-[5-(methylamino)-2-pyridyl]pyrrolo[2,3-b]pyridin-2-yl]methyl]-4-piperidyl]-N,N-bis(trideuteriomethyl)benzamide

Example 1k70 (2.8 mM in DMSO (0.80 mL)) was prepared similarly to Example 1k16 from 6-bromo-N-methylpyridin-3-amine (7 mg).

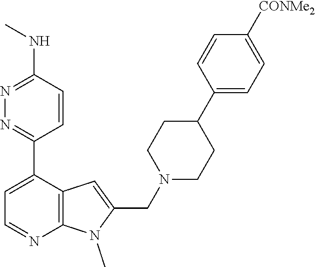

### Example 1k71. N,N-Dimethyl-4-[1-[[1-methyl-4-[6-(methylamino)pyridazin-3-yl]pyrrolo[2,3-b]pyridin-2-yl]methyl]-4-piperidyl]benzamide

Example 1k71 (10.0 mM in DMSO (1.54 mL)) was prepared similarly to Example 1k9 from Int. 1AB4 (13 mg) and 6-bromo-N-methyl-pyridazin-3-amine (10 mg).

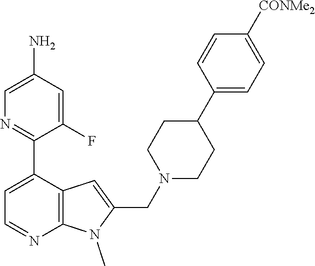

### Example 1k72. 4-[1-[[4-(5-Amino-3-fluoro-2-pyridyl)-1-methyl-pyrrolo[2,3-b]pyridin-2-yl]methyl]-4-piperidyl]-N,N-dimethyl-benzamide

Example 1k72 (10.0 mM in DMSO (1.24 mL)) was prepared similarly to Example 1k9 from Int. 1AB4 (13 mg) and 6-bromo-5-fluoro-pyridin-3-amine (10 mg).

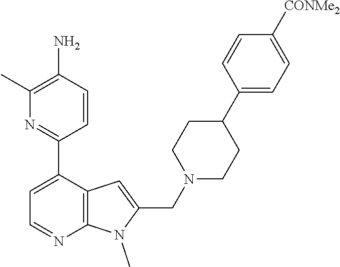

### Example 1k73. 4-[1-[[4-(5-Amino-6-methyl-2-pyridyl)-1-methyl-pyrrolo[2,3-b]pyridin-2-yl]methyl]-4-piperidyl]-N,N-dimethyl-benzamide

Example 1k73 (10.0 mM in DMSO (1.44 mL)) was prepared similarly to Example 1k9 from Int. 1AB4 (13 mg) and 6-bromo-2-methyl-pyridin-3-amine (10 mg).

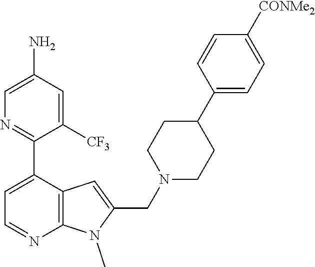

### Example 1k74. 4-[1-[[4-[5-Amino-3-(trifluoromethyl)-2-pyridyl]-1-methyl-pyrrolo[2,3-b]pyridin-2-yl]methyl]-4-piperidyl]-N,N-dimethyl-benzamide

Example 1k74 (10.0 mM in DMSO (1.19 mL)) was prepared similarly to Example 1k9 from Int. 1AB4 (13 mg) and 6-bromo-5-(trifluoromethyl)pyridin-3-amine (15 mg).

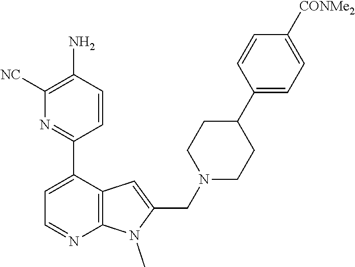

### Example 1k75. 4-(1-((4-(5-Amino-6-cyanopyridin-2-yl)-1-methyl-1H-pyrrolo[2,3-b]pyridin-2-yl)methyl)piperidin-4-yl)-N,N-dimethylbenzamide

Example 1k75 (10 mM in DMSO (2.20 mL)) was prepared similarly to Example 1ad1 from 3-amino-6-chloropicolinonitrile (18 mg).

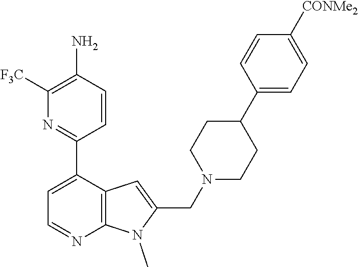

### Example 1k76. 4-[1-[[4-[5-Amino-6-(trifluoromethyl)-2-pyridyl]-1-methyl-pyrrolo[2,3-b]pyridin-2-yl]methyl]-4-piperidyl]-N,N-dimethyl-benzamide

Example 1k76 (10.0 mM in DMSO (2.70 mL)) was prepared similarly to Example 1k9 from Int. 1AB4 (20 mg) and 6-bromo-2-(trifluoromethyl)pyridin-3-amine (29 mg).

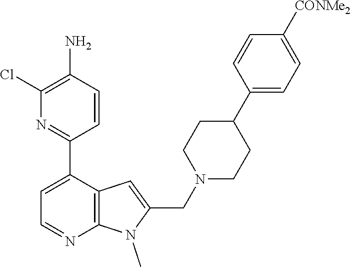

### Example 1k77. 4-(1-((4-(5-Amino-6-chloropyridin-2-yl)-1-methyl-1H-pyrrolo[2,3-b]pyridin-2-yl)methyl)piperidin-4-yl)-N,N-dimethylbenzamide

Example 1k77 (10 mM in DMSO (0.60 mL)) was prepared similarly to Example 1ad1 from ethyl 6-bromo-2-chloropyridin-3-amine (20 mg).

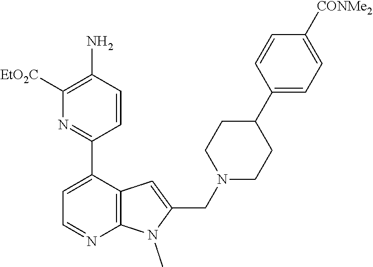

### Example 1k78. Ethyl 3-amino-6-(2-((4-(4-(dimethylcarbamoyl)phenyl)piperidin-1-yl)methyl)-1-methyl-1H-pyrrolo[2,3-b]pyridin-4-yl)picolinate

Example 1k78 (10 mM in DMSO (1.80 mL)) was prepared similarly to Example 1ad1 from ethyl 3-amino-6-bromopicolinate (58 mg).

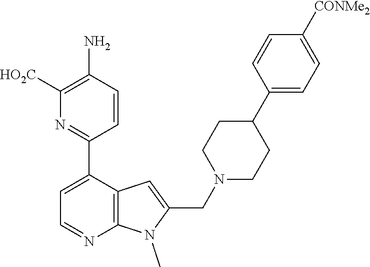

### Example 1k79. 3-Amino-6-[2-[[4-[4-(dimethylcarbamoyl)phenyl]-1-piperidyl]methyl]-1-methyl-pyrrolo[2,3-b]pyridin-4-yl]pyridine-2-carboxylic acid

Example 1k78 (18 mg) was stirred 2.5 h in MeOH/2M aq. NaOH (2:1, 3 mL) and purified by HPLC to give Example 1k79 (10.0 mM in DMSO (2.50 mL)).

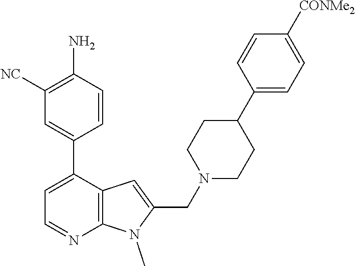

### Example 1k80. 4-(1-((4-(4-Amino-3-cyanophenyl)-1-methyl-1H-pyrrolo[2,3-b]pyridin-2-yl)methyl)piperidin-4-yl)-N,N-dimethylbenzamide

Int. 1AB4 (10 mg), 2-amino-5-bromo-benzonitrile (8 mg), Na₂CO₃ (6 mg), and PdDPPFCl₂-DCM (2 mg) were degassed in DMF/water (5:1, 1.2 ml) and stirred 1 h at 100° C. The mixture was filtered and HPLC-purified to give Example 1k80 (5 mg).

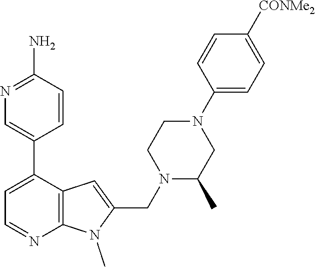

### Example 1n4. 4-[(3R)-4-[[4-(6-amino-3-pyridyl)-1-methyl-pyrrolo[2,3-b]pyridin-2-yl]methyl]-3-methyl-piperazin-1-yl]-N,N-dimethyl-benzamide

N,N-Dimethyl-4-[(3R)-3-methylpiperazin-1-yl]benzamide (78 mg, TFA salt), Int. 1AB8 (36 mg), DIPEA (0.13 mL), and 4A MS were stirred 1.5 h in DCM (1.5 mL). STAB (95 mg) was added and stirring continued 3 days. The OL (sat. aq. NaHCO₃/DCM) was dried, concentrated, and purified by FC (DCM/MeOH 1:0 to 9:1) to give 4-[(3R)-4-[(4-bromo-1-methyl-pyrrolo[2,3-b]pyridin-2-yl)methyl]-3-methyl-piperazin-1-yl]-N,N-dimethyl-benzamide (60 mg). This material, 5-(4,4,5,5-tetramethyl-1,3,2-dioxaborolan-2-yl)pyridin-2-amine (34 mg), PdDPPFCl₂-DCM (10 mg), and K₂CO₃ (53 mg) were degassed in DMF/water (5:1, 2.4 mL) and stirred ON at 100° C., filtered, and purified by HPLC to give Example 1n4 (10 mM in DMSO (1.7 mL)).

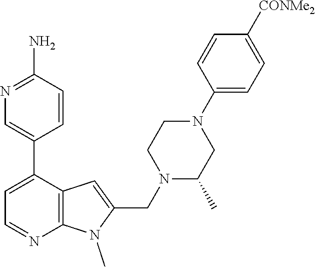

### Example 1n5. 4-[(3S)-4-[[4-(6-Amino-3-pyridyl)-1-methyl-pyrrolo[2,3-b]pyridin-2-yl]methyl]-3-methyl-piperazin-1-yl]-N,N-dimethyl-benzamide

Example 1n5 (10 mM in DMSO (1.89 mL)) was prepared similarly to Example 1n4 from N,N-dimethyl-4-[(3S)-3-methylpiperazin-1-yl]benzamide (78 mg, TFA salt).

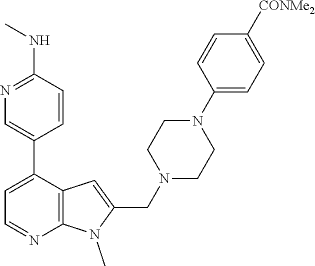

### Example 1n6. N,N-Dimethyl-4-[4-[[1-methyl-4-[6-(methylamino)-3-pyridyl]pyrrolo[2,3-b]pyridin-2-yl]methyl]piperazin-1-yl]benzamide

Int. 1AB8 (0.1 g), N,N-dimethyl-4-piperazin-1-yl-benzamide (0.18 g), and 4A MS were stirred 0.3 h in DCM (5 mL). STAB (0.27 g) was added and stirring continued ON. The mixture was filtered, concentrated, purified by SCX and by FC (heptane to MeOH/EtOAc 1:4) to give 4-[4-[(4-bromo-1-methyl-pyrrolo[2,3-b]pyridin-2-yl)-methyl]-piperazin-1-yl]-N,N-dimethyl-benzamide (0.19 g). 17 mg of this material, N-methyl-5-(4,4,5,5-tetra-methyl-1,3,2-dioxaborolan-2-yl)pyridin-2-amine (13 mg), PdDPPFCl₂-DCM (3 mg), and K₂CO₃ (15 mg) were degassed in DMF/water (10:1, 2.2 mL) and stirred 4 h at 100° C. The mixture was filtered and purified by HPLC to give Example 1n6 (14.6 mg).

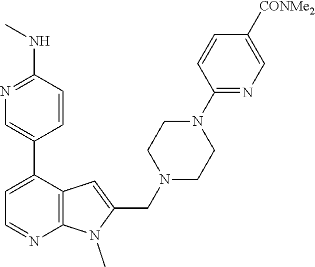

### Example 1n7. N,N-Dimethyl-6-[4-[[1-methyl-4-[6-(methylamino)-3-pyridyl]pyrrolo[2,3-b]pyridin-2-yl]methyl]piperazin-1-yl]pyridine-3-carboxamide

Int. 104 (10 mg) and N,N-dimethyl-6-(piperazin-1-yl)nicotinamide (15 mg, HCl salt) were stirred 0.5 h in DCE (0.5 mL). STAB (24 mg) was added and stirring was continued ON. The mixture was concentrated and HPLC-purified give Example 1n7 (6.7 mM in DMSO (0.70 mL)).

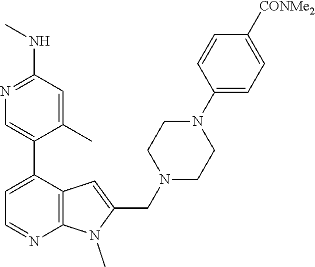

### Example 1n8. N,N-Dimethyl-4-[4-[[1-methyl-4-[4-methyl-6-(methylamino)-3-pyridyl]pyrrolo[2,3-b]pyridin-2-yl]methyl]piperazin-1-yl]benzamide

Int. 1N5 (13 mg), 5-bromo-N,4-dimethyl-pyridin-2-amine (9 mg), K₂CO₃ (12 mg), and PdDPPFCl₂-DCM (2 mg) were degassed in DMF/water (20:1, 2.1 mL) and stirred at 100° C. The mixture was filtered and purified by HPLC to give Example 1n8 (5.7 mg).

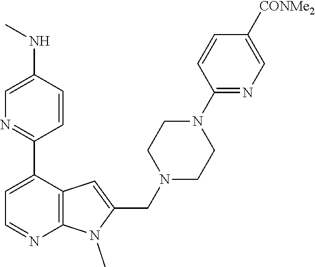

### Example 1n9. N,N-Dimethyl-6-[4-[[1-methyl-4-[5-(methylamino)-2-pyridyl]pyrrolo[2,3-b]pyridin-2-yl]methyl]piperazin-1-yl]pyridine-3-carboxamide

N,N-Dimethyl-6-(piperazin-1-yl)nicotinamide (15 mg, HCl salt), KOAc (7 mg), and Int. 1N6 (0.5 mL of a solution of 0.10 g in DCE (5 mL)) were stirred 0.5 h. STAB (24 mg) was added and stirring continued ON. The mixture was HPLC-purified to give Example 1n9 (6.9 mM in DMSO (0.85 mL)).

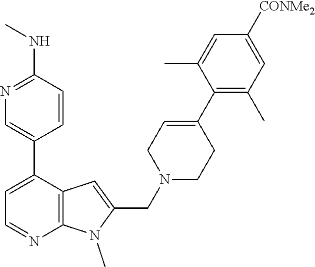

### Example 1o1. N,N,3,5-tetramethyl-4-[1-[[1-methyl-4-[5-(methylamino)-2-pyridyl]pyrrolo[2,3-b]pyridin-2-yl]methyl]-3,6-dihydro-2H-pyridin-4-yl]benzamide

Example 1o1 (10 mM in DMSO (1.6 mL)) was prepared similarly to Example 1n7 from Int. 1S1 (10 mg) and N,N,3,5-tetramethyl-4-(1,2,3,6-tetrahydro-pyridin-4-yl)benzamide (16 mg, HCl salt).

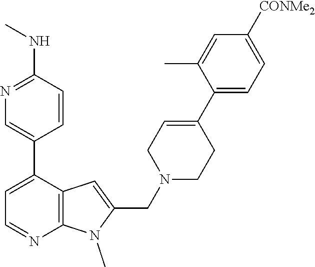

### Example 1o2. N,N,3-Trimethyl-4-[1-[[1-methyl-4-[5-(methylamino)-2-pyridyl]pyrrolo[2,3-b]pyridin-2-yl]methyl]-3,6-dihydro-2H-pyridin-4-yl]benzamide

Example 1o2 (6.2 mM in DMSO (0.7 mL)) was prepared similarly to Example 1n7 from Ints. 1S1/1AE8′ (10 mg/20 mg, TFA salt).

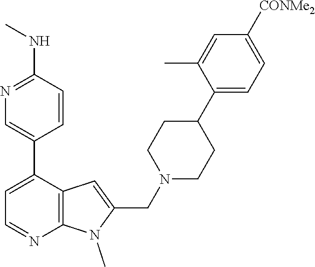

### Example 103. N,N,3-Trimethyl-4-[1-[[1-methyl-4-[5-(methylamino)-2-pyridyl]pyrrolo[2,3-b]pyridin-2-yl]methyl]-4-piperidyl]benzamide

Example 1o3 (1.3 mM in DMSO (0.85 mL)) was prepared similarly to Example 1n7 from Ints. 1S1/1AE8 (10 mg/20 mg, TFA salt).

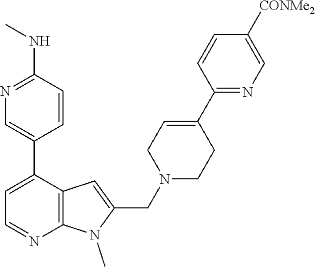

### Example 1o4. N,N-Dimethyl-6-[1-[[1-methyl-4-[5-(methylamino)-2-pyridyl]pyrrolo[2,3-b]pyridin-2-yl]methyl]-3,6-dihydro-2H-pyridin-4-yl]pyridine-3-carboxamide

Example 1o4 (6.1 mM in DMSO (0.85 mL)) was prepared similarly to Example 1n7 from Ints. 1S1/1S4 (10 mg/13 mg).

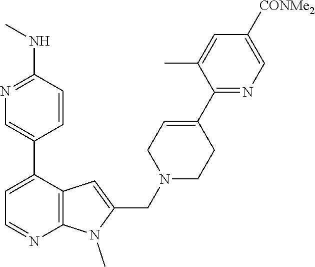

### Example 1o5. N,N,5-Trimethyl-6-[1-[[1-methyl-4-[5-(methylamino)-2-pyridyl]pyrrolo[2,3-b]pyridin-2-yl]methyl]-3,6-dihydro-2H-pyridin-4-yl]pyridine-3-carboxamide

Example 1o5 (4.5 mM in DMSO (0.85 mL)) was prepared similarly to Example 1n7 from Ints. 1S1/1S5 (10 mg/20 mg).

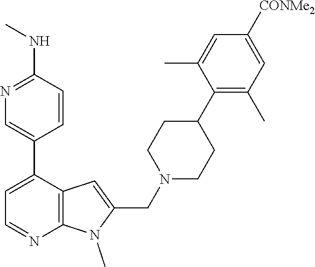

### Example 1o6. N,N,3,5-Tetramethyl-4-[1-[[1-methyl-4-[5-(methylamino)-2-pyridyl]pyrrolo[2,3-b]pyridin-2-yl]methyl]-4-piperidyl]benzamide

Example 1o6 (10.0 mM in DMSO (0.95 mL)) was prepared similarly to Example 1n7 from Ints. 1S1/1S6 (10 mg/13 mg).

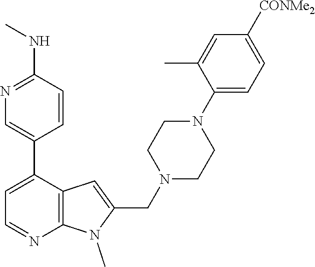

### Example 107. N,N,3-Trimethyl-4-[4-[[1-methyl-4-[5-(methylamino)-2-pyridyl]pyrrolo[2,3-b]pyridin-2-yl]methyl]piperazin-1-yl]benzamide

Example 1o7 (2.5 mM in DMSO (0.85 mL)) was prepared similarly to Example 1n7 from Ints. 1S1/1S7 (10 mg/14 mg).

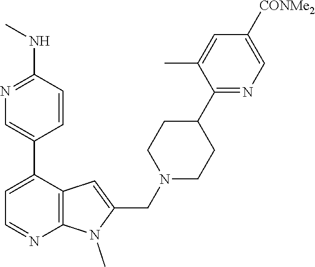

### Example 1o8. N,N,5-trimethyl-6-[1-[[1-methyl-4-[5-(methylamino)-2-pyridyl]pyrrolo[2,3-b]pyridin-2-yl]methyl]-4-piperidyl]pyridine-3-carboxamide

Example 1o8 (1.1 mM in DMSO (0.85 mL)) was prepared similarly to Example 1n7 from Int. 1S1/1S8 (10 mg/20 mg, TFA salt).

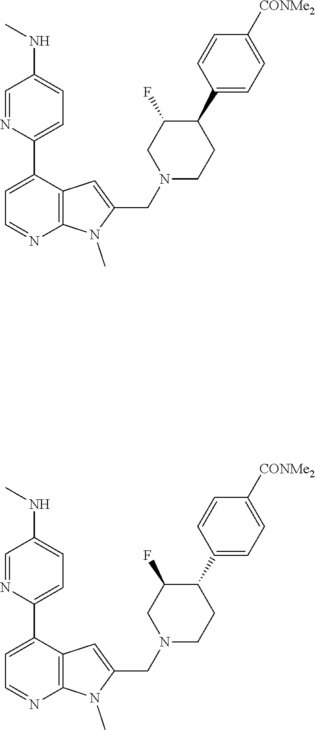

### Examples 1o17 Example 1o18. 4-((3S,4S)-3-fluoro-1-((1-methyl-4-(5-(methylamino)pyridin-2-yl)-1H-pyrrolo[2,3-b]pyridin-2-yl)methyl)piperidin-4-yl)-N,N-dimethylbenzamide and 4-((3R,4R)-3-fluoro-1-((1-methyl-4-(5-(methylamino)pyridin-2-yl)-1H-pyrrolo[2,3-b]pyridin-2-yl)methyl)piperidin-4-yl)-N,N-dimethylbenzamide

Examples 1o17 (8.5 mM in DMSO (0.80 mL)) and 1o18 (8.6 mM in DMSO (0.80 mL)) were prepared similarly to Example 1n7 from Ints. 104/1R9 (10 mg/12 mg) or Ints. 104/1R8 (10 mg/12 mg). The absolute configurations of these compounds were not determined.

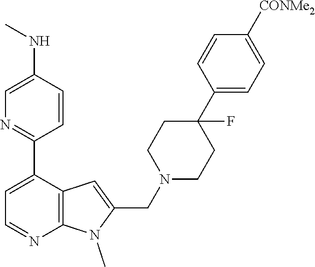

### Example 1o19. 4-[4-Fluoro-1-[[1-methyl-4-[5-(methylamino)-2-pyridyl]pyrrolo[2,3-b]pyridin-2-yl]methyl]-4-piperidyl]-N,N-dimethyl-benzamide

Example 1o19 (7.0 mM in DMSO (0.80 mL)) was prepared similarly to Example 1n7 from Int. 104/1R1 (10 mg/15 mg, HCl salt).

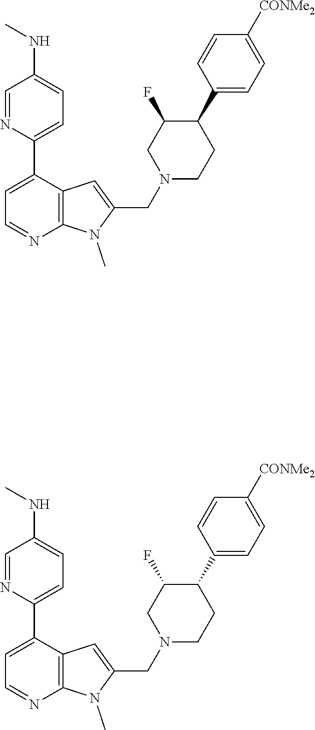

### Examples 1o20 and 1o21. N,N-Dimethyl-4-[(3S,4R)-3-fluoro-1-[[1-methyl-4-[5-(methylamino)-2-pyridyl]pyrrolo[2,3-b]pyridin-2-yl]methyl]-4-piperidyl]benzamide and N,N-dimethyl-4-[(3R,4S)-3-fluoro-1-[[1-methyl-4-[5-(methylamino)-2-pyridyl]pyrrolo[2,3-b]pyridin-2-yl]methyl]-4-piperidyl]benzamide

Example 1o20 (10 mM in DMSO (0.90 mL)) was prepared similarly to Example 1n7 from Ints. 104/1F1 (10 mg/15 mg). Example 1o21 (10 mM in DMSO (0.70 mL)) was prepared similarly from Ints. 104/1F2 (10 mg/15 mg). The absolute configurations of these compounds were not determined.

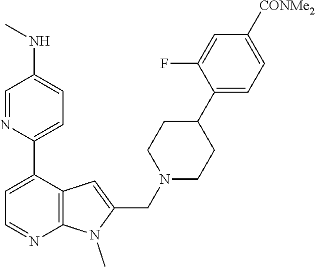

### Example 1o23. 3-Fluoro-N,N-dimethyl-4-[1-[[1-methyl-4-[5-(methylamino)-2-pyridyl]pyrrolo[2,3-b]pyridin-2-yl]methyl]-4-piperidyl]benzamide

Example 1o23 (9.1 mM in DMSO (0.80 mL)) was prepared similarly to Example 1n7 from Ints. 104/1S18 (10 mg/28 mg, HCl salt).

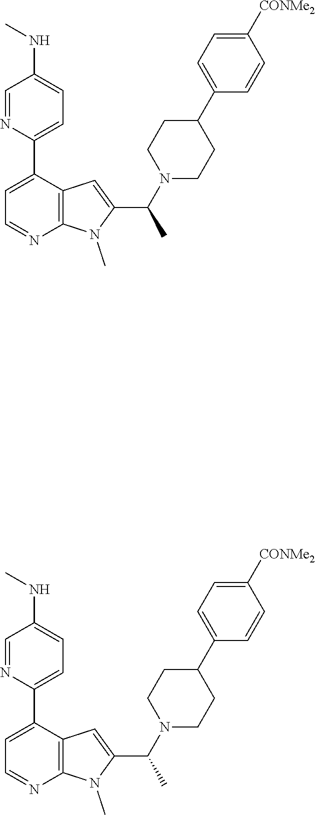

### Examples 1o24 and 1o25. (S)—N,N-dimethyl-4-(1-(1-(1-methyl-4-(5-(methylamino)pyridin-2-yl)-1H-pyrrolo[2,3-b]pyridin-2-yl)ethyl)piperidin-4-yl)benzamide and (R)—N,N-dimethyl-4-(1-(1-(1-methyl-4-(5-(methyl-amino)pyridin-2-yl)-1H-pyrrolo[2,3-b]pyridin-2-yl)ethyl)piperidin-4-yl)benzamide

Int. 1AF97 (19 mg) was resolved by SFC using a Chiralpak AD-H 250×10 5 μm column operated at 30° C. and an eluent of 55% CO₂ and 45% IPA (20 g/min) and a back pressure of 100 bar to give Example 1o24 (7 mg, first peak) and Example 1o25 (5.5 mg, second peak). The absolute configurations of these compounds were not determined.

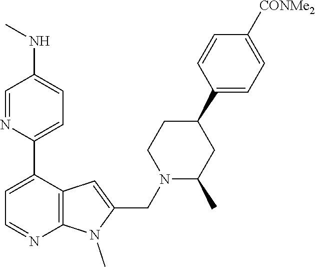

### Example 1o26. N,N-dimethyl-4-((2R,4R)-2-methyl-1-((1-methyl-4-(5-(methylamino)-pyridin-2-yl)-1H-pyrrolo[2,3-b]pyridin-2-yl)methyl)piperidin-4-yl)benzamide

Example 1o26 (15 mg) was prepared similarly to Example 1o28 from Ints. 101/1D4 (0.10 g/95 mg).

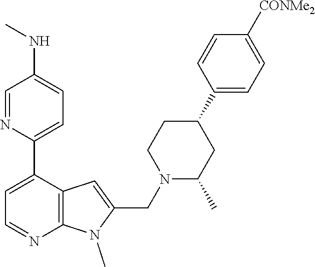

### Example 1o27. N,N-dimethyl-4-((2S,4S)-2-methyl-1-((1-methyl-4-(5-(methylamino)-pyridin-2-yl)-1H-pyrrolo[2,3-b]pyridin-2-yl)methyl)piperidin-4-yl)benzamide

Example 1o27 (28 mg) was prepared similarly to Example 1o28 from Ints. 101/1D3 (80 mg/66 mg).

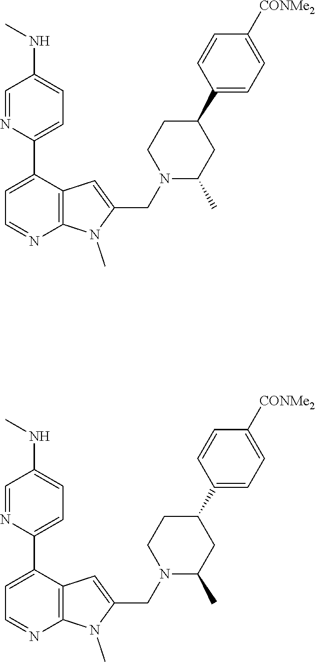

### Examples 1o28 and 1o29. N,N-dimethyl-4-((2S,4R)-2-methyl-1-((1-methyl-4-(5-(methylamino)pyridin-2-yl)-1H-pyrrolo[2,3-b]pyridin-2-yl)methyl)piperidin-4-yl)benzamide and N,N-dimethyl-4-((2R,4S)-2-methyl-1-((1-methyl-4-(5-(methylamino)pyridin-2-yl)-1H-pyrrolo[2,3-b]pyridin-2-yl)methyl)piperidin-4-yl)benzamide

Ints. 101/1D1 (90 mg/75 mg) and KI (4 mg) were stirred ON in DMF/DIPEA (20:1, 4.2 mL) at 0° C. to RT. The OL (water/MeOH/DCM 1:9) was dried, concentrated, stirred 3 h in DCM/TFA (38:1, 5.1 mL), and HPLC-purified to give Example 1o28 (15 mg). Example 1o29 (22 mg) was prepared similarly from Int. 1D2 (75 mg). The absolute configurations of these compounds were not determined.

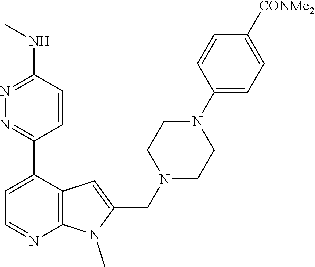

### Example 1p1. N,N-Dimethyl-4-[4-[[1-methyl-4-[6-(methylamino)pyridazin-3-yl]pyrrolo[2,3-b]pyridin-2-yl]methyl]piperazin-1-yl]benzamide

Int. 1P1 (90 mg), KI (4 mg), and N,N-dimethyl-4-piperazin-1-yl-benzamide (0.18 g, TFA salt) were stirred ON in DMF/DIPEA (16.7:1, 2.1 mL) at 0° C. to RT. The OL (EtOAc/water) was dried, concentrated, and purified by FC (DCM/MeOH 95:5) to give tert-butyl N-[6-[2-[[4-[4-(dimethyl-carbamoyl)phenyl]piperazin-1-yl]methyl]-1-methyl-pyrrolo[2,3-b]pyridin-4-yl]pyridazin-3-yl]-N-methyl-carbamate (80 mg). This material was stirred ON in DCM/TFA (45:1, 5.1 mL) at 0° C. to RT ON, concentrated, and purified by HPLC to give Example 1p1 (26 mg).

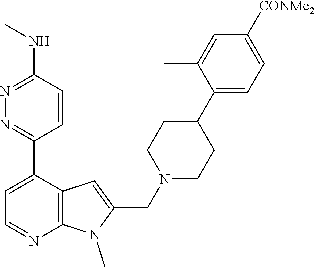

### Example 1p2. N,N,3-Trimethyl-4-[1-[[1-methyl-4-[6-(methylamino)pyridazin-3-yl]pyrrolo[2,3-b]pyridin-2-yl]methyl]-4-piperidyl]benzamide

Example 1p2 (9.1 mg and 9.49 mM in DMSO (1.0 mL)) was prepared similarly to Example 1p1 from Int. 1S7.

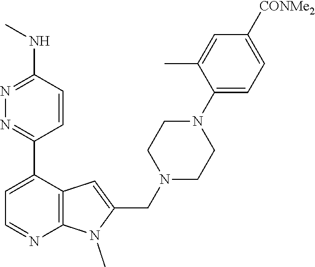

### Example 1p3. N,N,3-Trimethyl-4-[4-[[1-methyl-4-[6-(methylamino)pyridazin-3-yl]pyrrolo[2,3-b]pyridin-2-yl]methyl]piperazin-1-yl]benzamide

Example 1p3 (14.6 mg (TFA salt) and 6.1 mM in DMSO (1.0 mL)) was prepared similarly to Example 1p1 from Int. 1P2.

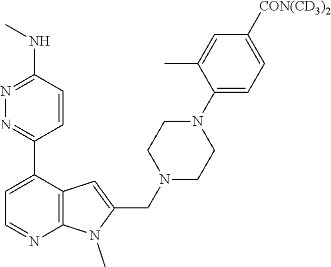

### Example 1p4. 3-Methyl-4-[4-[[1-methyl-4-[6-(methylamino)pyridazin-3-yl]pyrrolo[2,3-b]pyridin-2-yl]methyl]piperazin-1-yl]-N,N-bis(trideuteriomethyl)benzamide

Example 1p4 (10.0 mM in DMSO (1.8 mL)) was prepared similarly to Example 1p1 from Int. 1S7′.

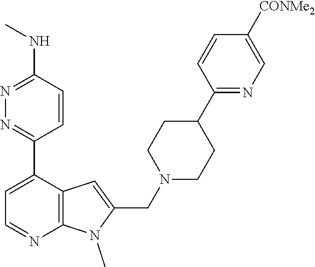

### Example 1p5. N,N-Dimethyl-6-[1-[[1-methyl-4-[6-(methylamino)pyridazin-3-yl]pyrrolo[2,3-b]pyridin-2-yl]methyl]-4-piperidyl]pyridine-3-carboxamide

Example 1p5 (15 mg) was prepared similarly to Example 1p1 from Int. 1M57 (0.18 g, TFA salt).

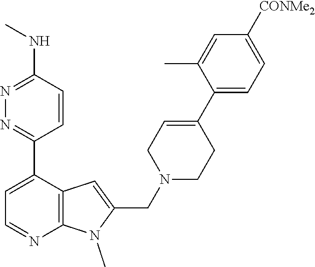

### Example 1p6. N,N,3-Trimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p6 (20 mg) was prepared similarly to Example 1p1 from Int. 1AE8′ (0.41 g, TFA salt).

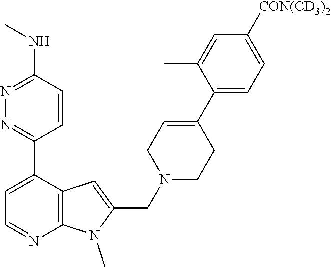

### Example 1p7. 3-Methyl-4-[1-[[1-methyl-4-[6-(methylamino)pyridazin-3-yl]pyrrolo[2,3-b]pyridin-2-yl]methyl]-3,6-dihydro-2H-pyridin-4-yl]-N,N-bis(trideuteriomethyl)benzamide

Example 1p7 (14 mg) was prepared similarly to Example 1p1 from 3-methyl-N,N-bis(methyl-d₃)-4-(1,2,3,6-tetrahydro-pyridin-4-yl)benzamide (30 mg, TFA salt).

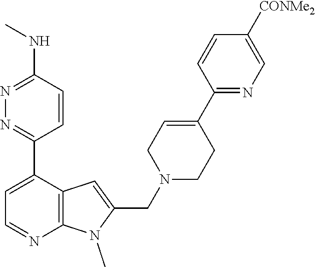

### Example 1p8. N,N-Dimethyl-6-[1-[[1-methyl-4-[6-(methylamino)pyridazin-3-yl]pyrrolo[2,3-b]pyridin-2-yl]methyl]-3,6-dihydro-2H-pyridin-4-yl]pyridine-3-carboxamide

Example 1p8 (17 mg) was prepared similarly to Example 1p1 from Int. 1S4 (0.22 g).

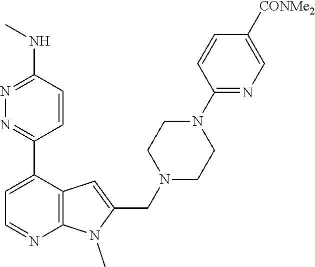

### Example 1p9. N,N-Dimethyl-6-[4-[[1-methyl-4-[6-(methylamino)pyridazin-3-yl]pyrrolo[2,3-b]pyridin-2-yl]methyl]piperazin-1-yl]pyridine-3-carboxamide

give Example 1p9 (17 mg) was prepared similarly to Example 1p1 from N,N-dimethyl-6-(piperazin-1-yl)-nicotinamide (0.22 g).

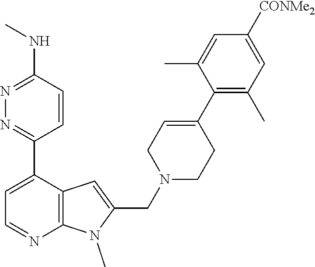

### Example 1p10. N,N,3,5-Tetramethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p10 (10 mM in DMSO (1.74 mL)) was prepared similarly to Example 1p1 from Int. 1S3 (24 mg).

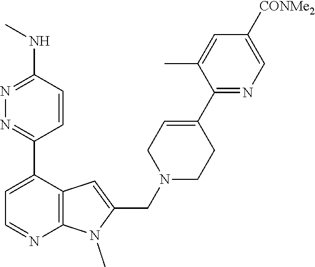

### Example 1p11. N,N,5-Trimethyl-6-[1-[[1-methyl-4-[6-(methylamino)pyridazin-3-yl]pyrrolo[2,3-b]pyridin-2-yl]methyl]-3,6-dihydro-2H-pyridin-4-yl]pyridine-3-carboxamide

Example 1p11 (10 mM in DMSO (0.83 mL)) was prepared similarly to Example 1p1 from Int. 1S5 (29 mg).

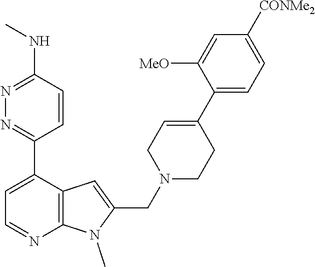

### Example 1p14. 3-Methoxy-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p14 (10 mM in DMSO (1.56 mL)) was prepared similarly to Example 1p1 from Int. 1M23 (22 mg).

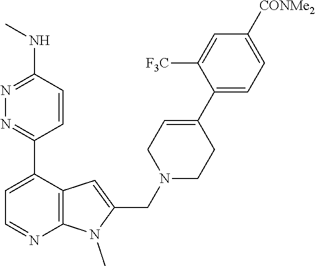

### Example 1p15. N,N-Dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]-3-(trifluoromethyl)benzamide

Example 1p15 (10 mM in DMSO (2.31 mL)) was prepared similarly to Example 1p1 from Int. 1M45 (24 mg).

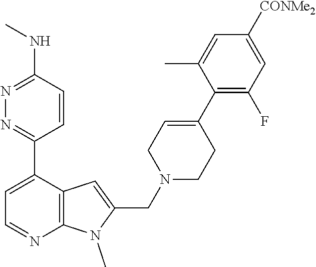

### Example 1p17. 3-Fluoro-N,N,5-trimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p17 (10.0 mM in DMSO (2.5 mL)) was prepared similarly to Example 1p1 from Int. 1M25.

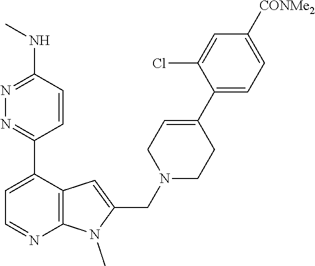

### Example 1p18. 3-Chloro-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p18 (10.0 mM in DMSO (2.5 mL)) was prepared similarly to Example 1p1 from Int. 1M43.

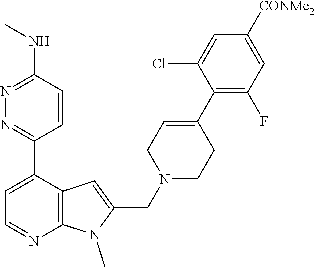

### Example 1p20. 3-Chloro-5-fluoro-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide

Example 1p20 (10 mM in DMSO (2.8 mL)) was prepared similarly to Example 1p1 from 3-chloro-5-fluoro-N,N-dimethyl-4-(1,2,3,6-tetrahydropyridin-4-yl)benzamide.

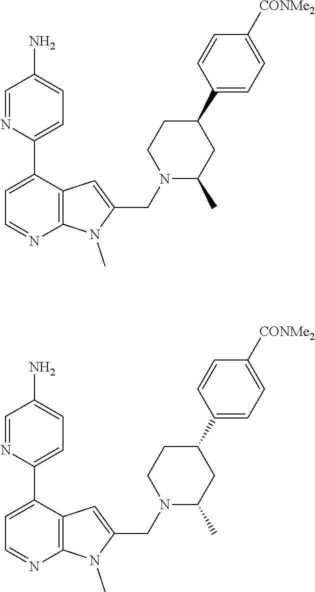

### Examples 1q1 and 1q6. N,N-Dimethyl-4-[(2R,4R)-1-[[4-(5-amino-2-pyridyl)-1-methyl-pyrrolo[2,3-b]pyridin-2-yl]methyl]-2-methyl-4-piperidyl]benzamide and N,N-dimethyl-4-[(2S,4S)-1-[[4-(5-amino-2-pyridyl)-1-methyl-pyrrolo[2,3-b]pyridin-2-yl]methyl]-2-methyl-4-piperidyl]benzamide

Examples 1q1 (3.9 mg) and 1q6 (17 mg) were prepared similarly to Example 1q3 from Ints. 1Q4/1D4 (76 mg/50 mg) or Ints. 1Q4/1D3 (80 mg/58 mg). The absolute configurations of these compounds were not determined.

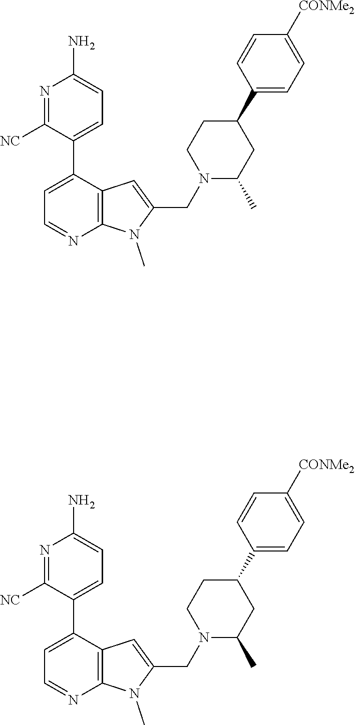

### Examples 1q2 and 1q3. N,N-Dimethyl-4-[(2S,4R)-1-[[4-(6-amino-2-cyano-3-pyridyl)-1-methyl-pyrrolo[2,3-b]pyridin-2-yl]methyl]-2-methyl-4-piperidyl]benzamide and N,N-dimethyl-4-[(2R,4S)-1-[[4-(6-amino-2-cyano-3-pyridyl)-1-methyl-pyrrolo[2,3-b]pyridin-2-yl]methyl]-2-methyl-4-piperidyl]benzamide

Example 1q2 (27 mg) was prepared similarly to Example 1q3 from Ints. 1Q1/1D2 (0.10 g/81 mg). Ints. 1Q1/1D1 (50 mg/40 mg) and KI (2 mg) were stirred ON in DMF/DIPEA (27:1, 3.1 mL). The OL (EtOAc/water) was dried, concentrated, stirred ON in DCM/TFA (107:1, 3 mL) at 0° C. to RT. The mixture was combined with another batch prepared similarly. The OL (water/DCM) was dried, concentrated, and HPLC-purified to give Example 1q3 (20 mg). The absolute configurations of these compounds were not determined.

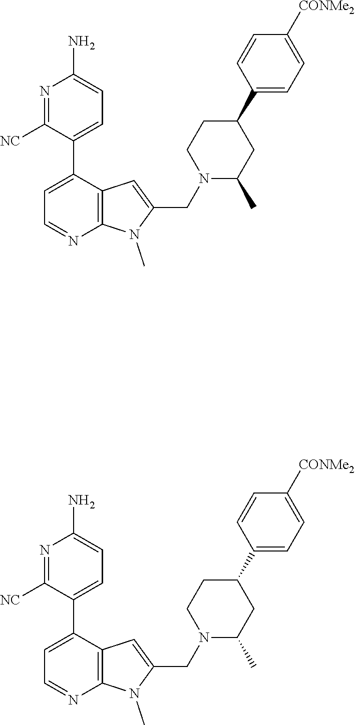

### Examples 1q4 and 1q5. N,N-Dimethyl-4-[(2R,4R)-1-[[4-(6-amino-2-cyano-3-pyridyl)-1-methyl-pyrrolo[2,3-b]pyridin-2-yl]methyl]-2-methyl-4-piperidyl]benzamide and N,N-dimethyl-4-[(2S,4S)-1-[[4-(6-amino-2-cyano-3-pyridyl)-1-methyl-pyrrolo[2,3-b]pyridin-2-yl]methyl]-2-methyl-4-piperidyl]benzamide

Example 1q4 (13 mg) was prepared similarly to Example 1q3 from Ints. 1Q1/1D4 (60 mg/45 mg). tert-Butyl N-tert-butoxycarbonyl-N-[6-cyano-5-[2-(hydroxymethyl)-1-methyl-pyrrolo[2,3-b]pyridin-4-yl]-2-pyridyl]-carbamate (0.22 g) was prepared similarly to Int. 1Q2 from tert-butyl N-(5-bromo-6-cyano-2-pyridyl)-N-tert-butoxycarbonyl-carbamate (0.25 g). 60 mg of this material was converted to tert-butyl N-tert-butoxy-carbonyl-N-[5-[2-(chloromethyl)-1-methyl-pyrrolo[2,3-b]pyridin-4-yl]-6-cyano-2-pyridyl]carbamate (60 mg) similarly to Int. 1Q1. This material and Int. 1D3 (33 mg) were converted to Example 1q5 (10 mg) similarly to Example 1q3. The absolute configurations of these compounds were not determined.

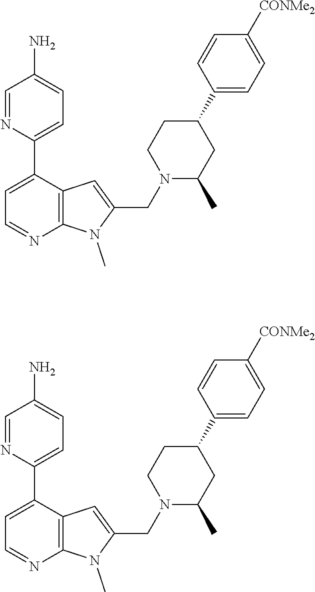

### Examples 1q7 and 1q8. N,N-Dimethyl-4-[(2R,4S)-1-[[4-(5-amino-2-pyridyl)-1-methyl-pyrrolo[2,3-b]pyridin-2-yl]methyl]-2-methyl-4-piperidyl]benzamide and N,N-dimethyl-4-[(2S,4R)-1-[[4-(5-amino-2-pyridyl)-1-methyl-pyrrolo[2,3-b]pyridin-2-yl]methyl]-2-methyl-4-piperidyl]benzamide

Examples 1q7 (17 mg) and 1q8 (11 mg) were prepared similarly to Example 1q3 from Ints. 1Q4/1D1 (0.10 g/86 mg) or Ints. 1Q4/1D2 (25 mg/21 mg). The absolute configurations of these compounds were not determined.

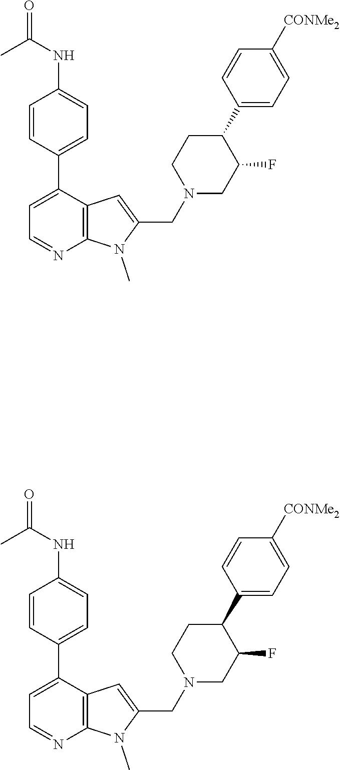

### Examples 1r5 and 1r6. N,N-Dimethyl-4-[(3S,4R)-1-[[4-(4-acetamidophenyl)-1-methyl-pyrrolo[2,3-b]pyridin-2-yl]methyl]-3-fluoro-4-piperidyl]benzamide and N,N-dimethyl-4-[(3R,4S)-1-[[4-(4-acetamidophenyl)-1-methyl-pyrrolo[2,3-b]pyridin-2-yl]methyl]-3-fluoro-4-piperidyl]benzamide

Int. 1R12 (60 mg), (4-acetamidophenyl)boronic acid (34 mg), NaHCO₃ (37 mg), and PdDPPFCl₂-DCM (10 mg) were degassed in dioxane/water (5:1, 1.2 mL) and stirred 1 h at 100° C. under MW conditions. The mixture was filtered, concentrated, and HPLC-purified to give Example 1r5 (28 mg). Example 1r6 (27 mg) was prepared similarly from Int. 1R13 (60 mg). The absolute configurations of these compounds were not determined.

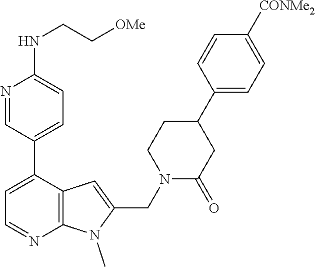

### Example 1s1. 4-[1-[[4-[6-(2-Methoxyethylamino)-3-pyridyl]-1-methyl-pyrrolo[2,3-b]pyridin-2-yl]methyl]-2-oxo-4-piperidyl]-N,N-dimethyl-benzamide

Prepared similarly to Example 1s2 from Int. 1A18 (7 mg) and N-(2-methoxyethyl)-5-(4,4,5,5-tetramethyl-1,3,2-dioxaborolan-2-yl)pyridin-2-amine (7 mg) to give Example 1s1 (10 mM in DMSO (0.95 mL)).

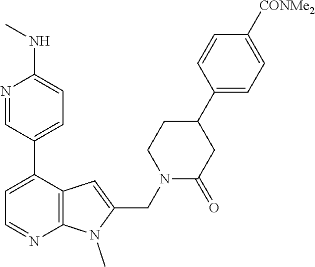

### Example 1s2. N,N-Dimethyl-4-[1-[[1-methyl-4-[6-(methylamino)-3-pyridyl]pyrrolo[2,3-b]pyridin-2-yl]methyl]-2-oxo-4-piperidyl]benzamide

Int. 1A18 (10 mg), N-methyl-5-(4,4,5,5-tetramethyl-1,3,2-dioxaborolan-2-yl)pyridin-2-amine (8 mg), PdDPPFCl₂-DCM (2 mg), and Na₂CO₃ (8 mg) were degassed in DMF/water (1:12, 13 mL) and stirred ON at 100° C. The mixture was filtered and purified by HPLC to give Example 1s2 (10 mM in DMSO (1.56 mL)).

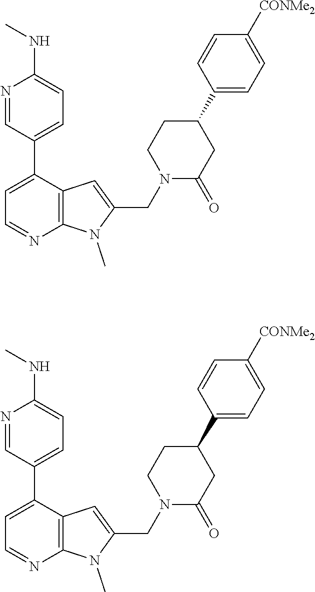

### Examples 1s3 and 1s4. N,N-Dimethyl-4-[(4R)-1-[[1-methyl-4-[6-(methylamino)-3-pyridyl]pyrrolo[2,3-b]pyridin-2-yl]methyl]-2-oxo-4-piperidyl]benzamide and N,N-dimethyl-4-[(4S)-1-[[1-methyl-4-[6-(methylamino)-3-pyridyl]pyrrolo[2,3-b]pyridin-2-yl]methyl]-2-oxo-4-piperidyl]benzamide

Example 1s3 (12 mg) was prepared similarly to Example 1s4 from Int. 1AB2 (13 mg) and N-methyl-5-(4,4,5,5-tetramethyl-1,3,2-dioxaborolan-2-yl)pyridin-2-amine (10 mg). Int. 1AB3 (10 mg), N-methyl-5-(4,4,5,5-tetra-methyl-1,3,2-dioxaborolan-2-yl)pyridin-2-amine (7 mg), PdDPPFCl₂-DCM (2 mg), and K₂CO₃ were degassed in DMF/water (3.8:1, 0.6 mL) and stirred 2 h at 100° C., filtered, and HPLC-purified to give Example 1s4 (10 mM in DMSO (1.12 mL)). The absolute configurations of these compounds were not determined.

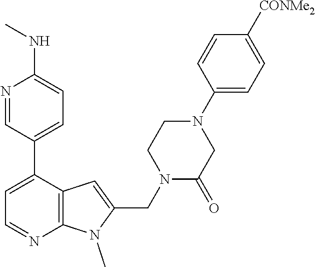

### Example 1s5. N,N-Dimethyl-4-[4-[[1-methyl-4-[6-(methylamino)-3-pyridyl]pyrrolo[2,3-b]pyridin-2-yl]methyl]-3-oxo-piperazin-1-yl]benzamide

Int. 1AE16 (0.10 g), N-methyl-5-(4,4,5,5-tetramethyl-1,3,2-dioxaborolan-2-yl)pyridin-2-amine (75 mg), NaHCO₃ (54 mg), and PdDPPFCl₂-DCM (17 mg) were degassed in dioxane/water (10:1, 3.3 mL) and stirred 1.5 h at 130° C. The mixture was concentrated and purified by FC (DCM/MeOH 9:1) and HPLC to give Example 1s5 (15 mg).

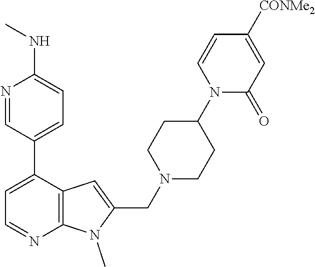

### Example 1s6. N,N-Dimethyl-1-[1-[[1-methyl-4-[6-(methylamino)-3-pyridyl]pyrrolo[2,3-b]pyridin-2-yl]methyl]-4-piperidyl]-2-oxo-pyridine-4-carboxamide

Ints. 1S1 (0.10 g) and 1S2 (0.11 g) were stirred ON in DCE/DIPEA (16.7:1, 5.3 mL). STAB (0.16 g) was added and stirring continued 2 h. The OL (water/DCM/MeOH 9:1) was dried, concentrated, and purified by FC (DCM/MeOH 9:1) and HPLC to give Example 1S6 (7 mg).

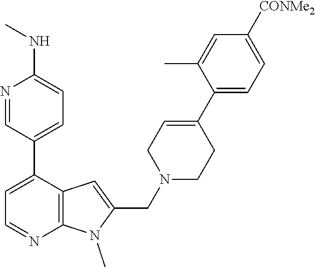

### Example 1s8. N,N,3-Trimethyl-4-[1-[[1-methyl-4-[6-(methylamino)-3-pyridyl]pyrrolo[2,3-b]pyridin-2-yl]methyl]-3,6-dihydro-2H-pyridin-4-yl]benzamide

Ints. 1AB6/1S1 (20 mg/0.5 mL of a solution of 0.1 g in 5 mL DCE), and KOAc (7 mg) were stirred 0.5 h. STAB (24 mg) was added and stirring continued ON. The mixture was concentrated and purified by HPLC to give Example 1s8 (6.23 mM in DMSO (0.70 mL)).

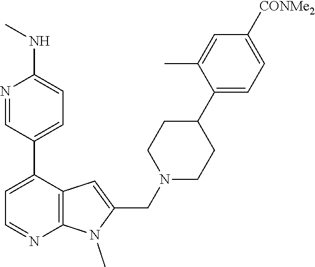

### Example 1s9. N,N,3-Trimethyl-4-[1-[[1-methyl-4-[6-(methylamino)-3-pyridyl]pyrrolo[2,3-b]pyridin-2-yl]methyl]-4-piperidyl]benzamide

Example 1s9 (7.35 mM in DMSO (0.70 mL)) was prepared similarly to Example 1s8 from Ints. 1S1/1AE8 (20 mg, TFA salt/10 mg).

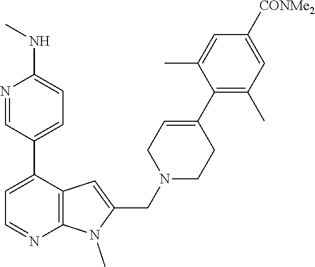

### Example 1s10. N,N,3,5-Tetramethyl-4-[1-[[1-methyl-4-[6-(methylamino)-3-pyridyl]pyrrolo[2,3-b]pyridin-2-yl]methyl]-3,6-dihydro-2H-pyridin-4-yl]benzamide

Example 1s10 (10 mM in DMSO (1.60 mL)) was prepared similarly to Example 1s8 from Ints. 1S1/1S3 (10 mg/17 mg).

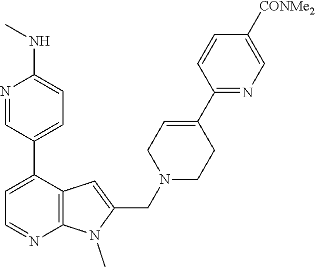

### Example 1s11. N,N-Dimethyl-6-[1-[[1-methyl-4-[6-(methylamino)-3-pyridyl]pyrrolo[2,3-b]pyridin-2-yl]methyl]-3,6-dihydro-2H-pyridin-4-yl]pyridine-3-carboxamide

Example 1s11 (10 mM in DMSO (0.85 mL)) was prepared similarly to Example 1s8 from Ints. 1S1/1S4 (10 mg/15 mg).

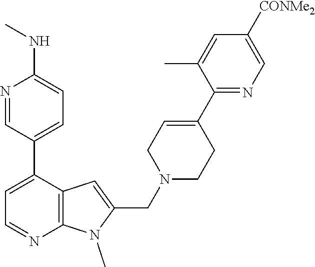

### Example 1s12. N,N,5-Trimethyl-6-[1-[[1-methyl-4-[6-(methylamino)-3-pyridyl]pyrrolo[2,3-b]pyridin-2-yl]methyl]-3,6-dihydro-2H-pyridin-4-yl]pyridine-3-carboxamide

Example 1s12 (10 mM in DMSO (0.89 mL)) was prepared similarly to Example 1s8 from Ints. 1S1/1S5 (10 mg/20 mg).

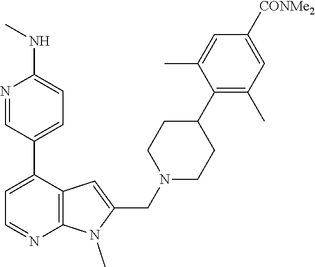

### Example 1s13. N,N,3,5-Tetramethyl-4-[1-[[1-methyl-4-[6-(methylamino)-3-pyridyl]pyrrolo[2,3-b]pyridin-2-yl]methyl]-4-piperidyl]benzamide

Example 1s13 (5.13 mM in DMSO (0.70 mL)) was prepared similarly to Example 1s8 from Ints. 1S1/1S6 (10 mg/16 mg).

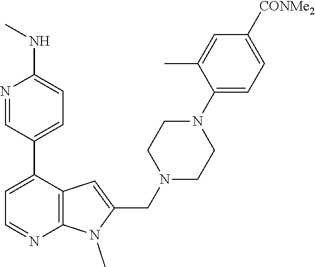

### Example 1s14. N,N,3-Trimethyl-4-[4-[[1-methyl-4-[6-(methylamino)-3-pyridyl]pyrrolo[2,3-b]pyridin-2-yl]methyl]piperazin-1-yl]benzamide

Example 1s14 (4.06 mM in DMSO (0.70 mL)) was prepared similarly to Example 1s8 from Ints. 1S1/1S7 (10 mg/16 mg).

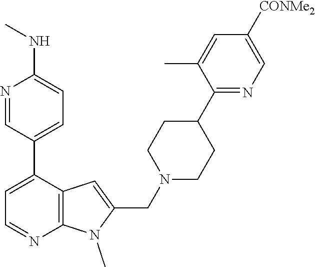

### Example 1s15. N,N,5-Trimethyl-6-[1-[[1-methyl-4-[6-(methylamino)-3-pyridyl]pyrrolo[2,3-b]pyridin-2-yl]methyl]-4-piperidyl]pyridine-3-carboxamide

Example 1s15 (1.18 mM in DMSO (0.70 mL)) was prepared similarly to Example 1s8 from Ints. 1S1/1S8 (10 mg/16 mg).

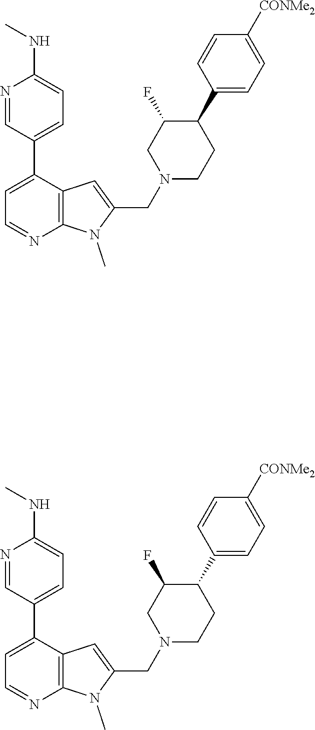

### Examples 1s24 and 1s25. N,N-Dimethyl-4-[(3R,4R)-3-fluoro-1-[[1-methyl-4-[6-(methylamino)-3-pyridyl]pyrrolo[2,3-b]pyridin-2-yl]methyl]-4-piperidyl]benzamide and N,N-dimethyl-4-[(3S,4S)-3-fluoro-1-[[1-methyl-4-[6-(methylamino)-3-pyridyl]pyrrolo[2,3-b]pyridin-2-yl]methyl]-4-piperidyl]benzamide

Examples 1s24 (9.95 mM in DMSO (0.97 mL)) and 1s25 (10 mM in DMSO (0.93 mL)) were prepared similarly to Example 1s26 from Ints. 1S1/1R8 (8 mg/20 mg) or Ints. 1S1/1R9 (8 mg/20 mg). The absolute configurations of these compounds were not determined.

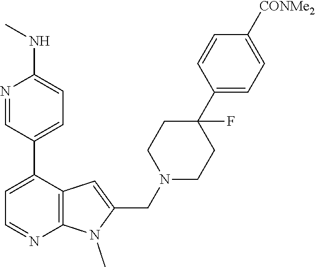

### Example 1s26. 4-[4-Fluoro-1-[[1-methyl-4-[6-(methylamino)-3-pyridyl]pyrrolo[2,3-b]pyridin-2-yl]methyl]-4-piperidyl]-N,N-dimethyl-benzamide

Ints. 1S1/1R1 (8 mg/11 mg) and KOAc (9 mg) were stirred 0.5 h in DCE (0.5 mL). STAB (28 mg) was added and stirring continued ON. The mixture was HPLC-purified to give Example 1s26 (9.68 mM in DMSO (0.80 mL)).

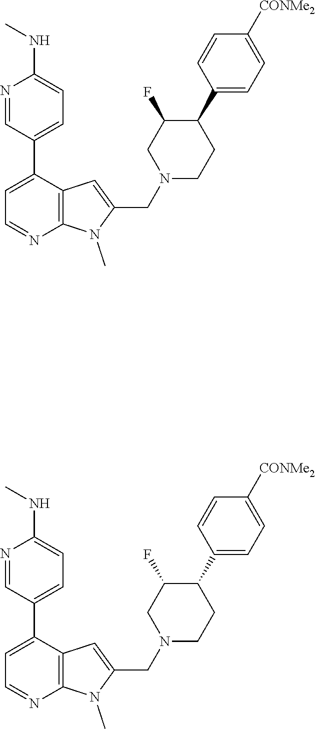

### Examples 1s27 and 1s28. N,N-Dimethyl-4-[(3R,4S)-3-fluoro-1-[[1-methyl-4-[6-(methylamino)-3-pyridyl]pyrrolo[2,3-b]pyridin-2-yl]methyl]-4-piperidyl]benzamide and N,N-dimethyl-4-[(3S,4R)-3-fluoro-1-[[1-methyl-4-[6-(methylamino)-3-pyridyl]pyrrolo[2,3-b]pyridin-2-yl]methyl]-4-piperidyl]benzamide

Examples 1s27 (9.97 mM in DMSO (1.26 mL)) and 1s28 (10 mM in DMSO (0.99 mL)) were prepared similarly to Example 1s26 from Ints. 1S1/1F2 (8 mg/20 mg) or Ints. 1S1/1F1 (8 mg/20 mg). The absolute configurations of these compounds were not determined.

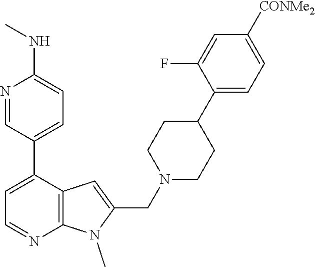

### Example 1s30. 3-Fluoro-N,N-dimethyl-4-(1-((1-methyl-4-(6-(methylamino)pyridin-3-yl)-1H-pyrrolo[2,3-b]pyridin-2-yl)methyl)piperidin-4-yl)benzamide

Example 1s30 (9.92 mM in DMSO (0.80 mL)) was prepared similarly to Example 1s26 from Ints. 1S1/1S18 (8 mg/20 mg).

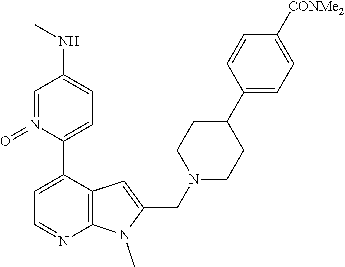

### Example 1u2. N,N-Dimethyl-4-[1-[[1-methyl-4-[5-(methylamino)-1-oxido-pyridin-1-ium-2-yl]pyrrolo[2,3-b]pyridin-2-yl]methyl]-4-piperidyl]benzamide

Int. 1AB1 (62 mg), tert-butyl N-methyl-N-(1-oxo-3-pyridyl)carbamate (61 mg), Pd(OAc)₂ (3 mg), and P(tBu)₃-HBF₄ (5 mg) were degassed in toluene (0.34 mL) and stirred 0.25 h and 24 h at 120° C. The mixture was purified by FC (hexane to MeOH), stirred 1 h in DCM/TFA (1:1, 2 mL), concentrated, and HPLC-purified to give Example 1u2 (10 mM in DMSO (1.94 mL)).

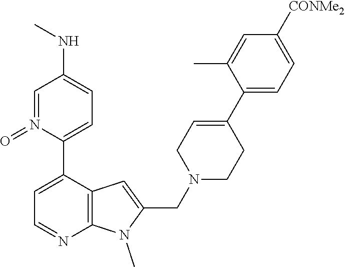

### Example 1u3. N,N,3-Trimethyl-4-[1-[[1-methyl-4-[5-(methylamino)-1-oxido-pyridin-1-ium-2-yl]pyrrolo[2,3-b]pyridin-2-yl]methyl]-3,6-dihydro-2H-pyridin-4-yl]benzamide

Example 1u3 (10 mM in DMSO (0.69 mL)) was prepared similarly to Example 1u2 from tert-butyl N-methyl-N-(1-oxo-3-pyridyl)carbamate (76 mg) and Int. 1AB5 (79 mg).

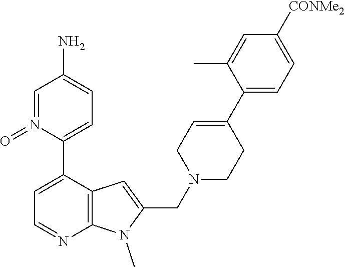

### Example 1u5. 4-[1-[[4-(5-Amino-1-oxido-pyridin-1-ium-2-yl)-1-methyl-pyrrolo[2,3-b]pyridin-2-yl]methyl]-4-piperidyl]-N,N-dimethyl-benzamide

Example 1u5 (10 mM in DMSO (0.35 mL)) was prepared similarly to Examples 1u2 from tert-butyl N-tert-butoxycarbonyl-N-(1-oxo-3-pyridyl)carbamate (0.11 g) and Int. 1AB1 (80 mg).

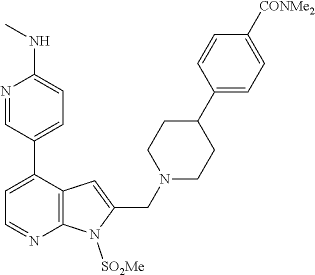

### Example 1x1. N,N-Dimethyl-4-[1-[[4-[6-(methylamino)-3-pyridyl]-1-methylsulfonyl-pyrrolo[2,3-b]pyridin-2-yl]methyl]-4-piperidyl]benzamide

Int. 1X1 (12 mg) and NaH (2 mg) were stirred in DMF (2 mL) for 10 minutes. MsCl (5 μL) was added and stirring was continued 72 h. More NaH (4 mg) and MsCl (5 μL) was added after 16 h and 36 h and stirring continued ON. The mixture was purified by HPLC to give 4-[1-[(4-bromo-1-methylsulfonyl-pyrrolo[2,3-b]-pyridin-2-yl)methyl]-4-piperidyl]-N,N-dimethyl-benzamide. 12.4 mg of this material, N-methyl-5-(4,4,5,5-tetramethyl-1,3,2-dioxaborolan-2-yl)pyridin-2-amine (8.4 mg), KOAc (10 mg), and PdDPPFCl₂-DCM (1 mg) were degassed in DMF/water (10:1, 2.2 mL) and stirred ON at 70° C. The mixture was filtered and purified by HPLC to give Example 1x1 (3.5 mg).

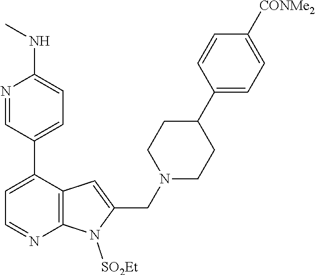

### Example 1x2. 4-[1-[[1-Ethylsulfonyl-4-[6-(methylamino)-3-pyridyl]pyrrolo[2,3-b]pyridin-2-yl]methyl]-4-piperidyl]-N,N-dimethyl-benzamide

Example 1x2 (3.9 mg) was prepared similarly to Example lxi from Int. 1X1 (23 mg) and ethanesulfonyl chloride (49 μL).

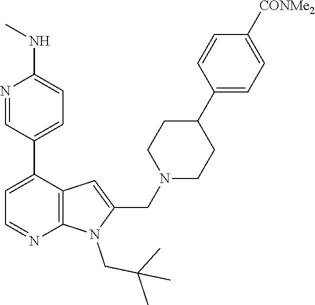

### Example 1×3. 4-[1-[[1-(2,2-Dimethylpropyl)-4-[6-(methylamino)-3-pyridyl]pyrrolo[2,3-b]pyridin-2-yl]methyl]-4-piperidyl]-N,N-dimethyl-benzamide

Int. 1AD2 (0.10 g), Cs₂CO₃ (0.58 g), and 1-bromo-2,2-dimethyl-propane (0.53 g) were stirred ON in DMF (2 mL) at 0° C. to 80° C. The mixture was filtered and concentrated to give tert-butyl N-[5-[2-[[4-[4-(dimethyl-carbamoyl)phenyl]-1-piperidyl]methyl]-1-(2,2-dimethylpropyl)-pyrrolo[2,3-b]pyridin-4-yl]-2-pyridyl]-N-methyl-carbamate, 0. 1 g of this material was stirred 4 h in DCM/TFA (333:1, 5 mL) at 0° C. to RT, concentrated, and purified by HPLC to give Example 1x3 (1 1 mg).

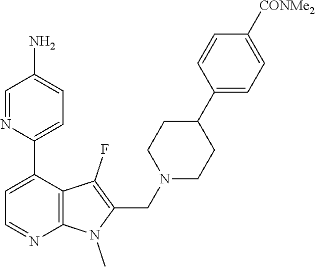

### Example 1y1. 4-(1-((4-(5-Aminopyridin-2-yl)-3-fluoro-1-methyl-1H-pyrrolo[2,3-b]pyridin-2-yl)methyl)piperidin-4-yl)-N,N-dimethylbenzamide

Ints. 1Y1/1Y14 (0.12 g/77 mg), K₂CO₃ (61 mg), and Pd(PPh₃)₂Cl₂ (14 mg) were degassed in dioxane/water (7:3, 10 mL) and stirred ON at 100° C. and filtered. The OL (water/EtOAc) was concentrated. The residue was washed with pentane to give tert-butyl N-[6-[2-[[4-[4-(dimethylcarbamoyl)-phenyl]-1-piperidyl]methyl]-3-fluoro-1-methyl-pyrrolo[2,3-b]pyridin-4-yl]-3-pyridyl]-N-methyl-carbamate. This material was stirred ON in DCM/TFA (40:1, 20.5 mL) at 0° C. to RT, concentrated, and HPLC-purified to give Example 1y1 (11 mg).

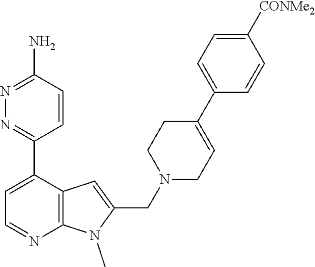

### Example 1z1. 4-(1-((4-(6-aminopyridazin-3-yl)-1-methyl-1H-pyrrolo[2,3-b]pyridin-2-yl)methyl)-1,2,3,6-tetrahydropyridin-4-yl)-N,N-dimethylbenzamide

Int. 1Z12 (0.75 g) was stirred 0.5 h in THF/2.8M HCl in dioxane (2:1, 30 mL). The mixture was concentrated and HPLC-purified to give Example 1z1 (16 mg).

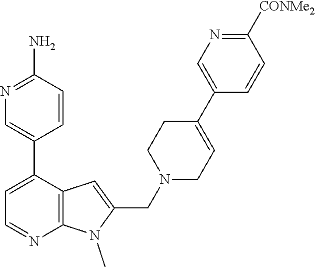

### Example 1z2. 1′-((4-(6-aminopyridin-3-yl)-1-methyl-1H-pyrrolo[2,3-b]pyridin-2-yl)methyl)-N,N-dimethyl-1′,2′,3′,6′-tetrahydro-[3,4′-bipyridine]-6-carboxamide

Example 1z2 (35 mg) was prepared similarly to Example 1z1 from Int. 1Z13 (0.20 g).

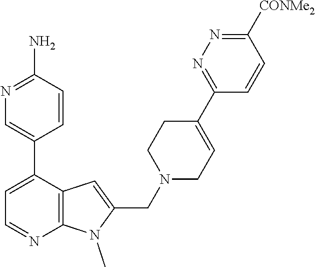

### Example 1z3. 6-(1-((4-(6-aminopyridin-3-yl)-1-methyl-1H-pyrrolo[2,3-b]pyridin-2-yl)methyl)-1,2,3,6-tetrahydropyridin-4-yl)-N,N-dimethylpyridazine-3-carboxamide

PdDPPFCl₂-DCM (10 mg), Int. 1Z14 (70 mg), (6-aminopyridin-3-yl)boronic acid (50 mg), and K₂CO₃ (70 mg) were stirred 12 h in dioxane/H₂O (5:1, 6 mL) at 80° C. under an inert atmosphere. The OL (water/EtOAc) was concentrated and HPLC-purified to give Example 1z3 (30 mg).

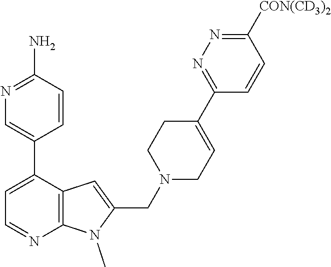

### Example 1z4. 6-(1-((4-(6-aminopyridin-3-yl)-1-methyl-1H-pyrrolo[2,3-b]pyridin-2-yl)methyl)-1,2,3,6-tetrahydropyridin-4-yl)-N,N-bis(methyl-d₃)pyridazine-3-carboxamide

Example 1z4 (16 mg) was prepared similarly to Example 1z3 from Int. 1Z17 (50 mg).

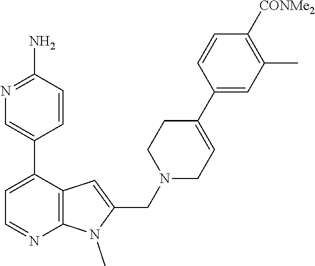

### Example 1z5. 4-(1-((4-(6-aminopyridin-3-yl)-1-methyl-1H-pyrrolo[2,3-b]pyridin-2-yl)methyl)-1,2,3,6-tetrahydropyridin-4-yl)-N,N,2-trimethylbenzamide

Example 1z5 (0.15 g) was prepared similarly to Example 1z3 from Int. 1Z62 (0.19 g) and 5-(4,4,5,5-tetramethyl-1,3,2-dioxaborolan-2-yl)pyridin-2-amine (0.11 g).

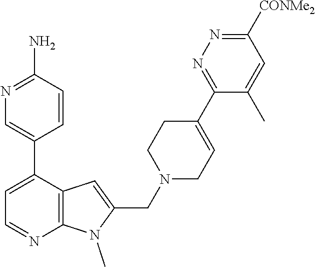

### Example 1z6. 6-(1-((4-(6-aminopyridin-3-yl)-1-methyl-1H-pyrrolo[2,3-b]pyridin-2-yl)methyl)-1,2,3,6-tetrahydropyridin-4-yl)-N,N,5-trimethylpyridazine-3-carboxamide

Example 1z6 (70 mg) was prepared similarly to Example 1z3 from Int. 1Z57 (0.19 g) and 5-(4,4,5,5-tetramethyl-1,3,2-dioxaborolan-2-yl)pyridin-2-amine (0.13 g).

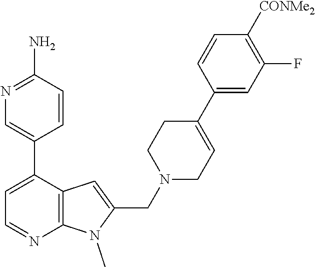

### Example 1z7. 4-(1-((4-(6-aminopyridin-3-yl)-1-methyl-1H-pyrrolo[2,3-b]pyridin-2-yl)methyl)-1,2,3,6-tetrahydropyridin-4-yl)-2-fluoro-N,N-dimethylbenzamide

Example 1z7 (50 mg) was prepared similarly to Example 1z1 from Int. 1Z21 (0.20 g).

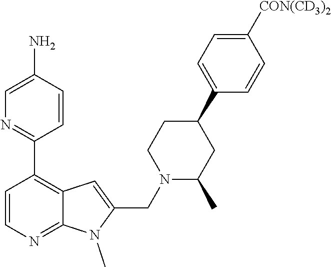

### Example 1z9. 4-((2R,4R)-1-((4-(5-aminopyridin-2-yl)-1-methyl-1H-pyrrolo[2,3-b]pyridin-2-yl)methyl)-2-methylpiperidin-4-yl)-N,N-bis(methyl-d₃)benzamide

Int. 1Z22 (0.12 g) was stirred 16 h in DCM/TFA (64:1, 10.6 mL). The mixture was concentrated. The OL (aq. Na₂CO₃/DCM/MeOH) dried, filtered, concentrated, and purified by FC (DCM/MeOH 9:1) to give Example 1z9 (42 mg).

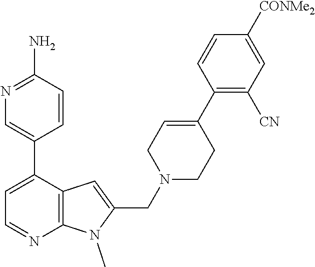

### Example 1z10. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyano-N,N-dimethylbenzamide

Int. 1Z23 (60 mg) was stirred ON in 2.8M HCl in dioxane (10 mL). The mixture was concentrated and HPLC-purified to give Example 1z10 (9 mg).

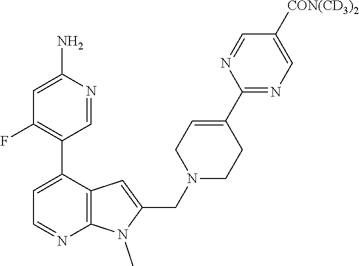

### Example 1z11. 2-(1-((4-(6-amino-4-fluoropyridin-3-yl)-1-methyl-1H-pyrrolo[2,3-b]pyridin-2-yl)methyl)-1,2,3,6-tetrahydropyridin-4-yl)-N,N-bis(methyl-d₃)pyrimidine-5-carboxamide

Example 1z11 (11 mg) was prepared similarly to Example 1z3 from Int. 1AF3 (45 mg) and 4-fluoro-5-(4,4,5,5-tetramethyl-1,3,2-dioxaborolan-2-yl)pyridin-2-amine (40 mg).

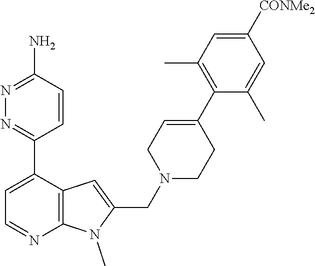

### Example 1z12. 4-(1-((4-(6-aminopyridazin-3-yl)-1-methyl-1H-pyrrolo[2,3-b]pyridin-2-yl)methyl)-1,2,3,6-tetrahydropyridin-4-yl)-N,N,3,5-tetramethylbenzamide

Int. 1Z28 (0.3 g) was stirred ON in MeOH/2.8M HCl in dioxane (10:1, 11 mL). The mixture was concentrated and HPLC-purified to give Example 1z12 (37 mg).

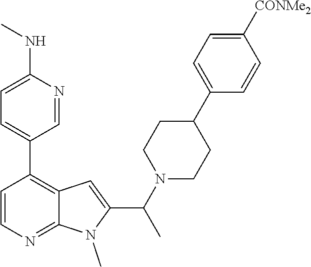

### Example 1z13. N,N-dimethyl-4-(1-(1-(1-methyl-4-(6-(methylamino)pyridin-3-yl)-1H-pyrrolo[2,3-b]pyridin-2-yl)ethyl)piperidin-4-yl)benzamide

Example 1z13 (35 mg) was prepared similarly to Example 1z3 from Int. 1AF17 (60 mg) and N-methyl-5-(4,4,5,5-tetramethyl-1,3,2-dioxaborolan-2-yl)pyridin-2-amine (45 mg).

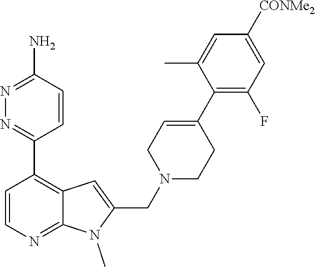

### Example 1z14. 4-(1-((4-(6-aminopyridazin-3-yl)-1-methyl-1H-pyrrolo[2,3-b]pyridin-2-yl)methyl)-1,2,3,6-tetrahydropyridin-4-yl)-3-fluoro-N,N,5-trimethylbenzamide

Example 1z14 (0.13 g) was prepared similarly to Example 1z16 from Int. 1Z31 (0.50 g) and 6-bromopyridazin-3-amine (0.19 g) using [dicyclohexyl(2′,6′-diisopropoxy-2-biphenylyl)-phosphine-κP](methanesulfonatato-κO)[2′-(methylamino-κN)-2-biphenylyl-κC²]palladium (90 mg) as catalyst.

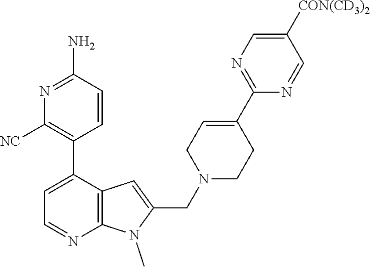

### Example 1z15. 2-(1-((4-(6-amino-2-cyanopyridin-3-yl)-1-methyl-1H-pyrrolo[2,3-b]pyridin-2-yl)methyl)-1,2,3,6-tetrahydropyridin-4-yl)-N,N-bis(methyl-d₃)pyrimidine-5-carboxamide

Example 1z14 (9 mg) was prepared similarly to Example 1z3 from Ints. 1Z32/1AF3 (40 mg/45 mg).

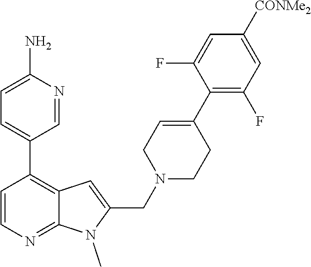

### Example 1z16. 4-(1-((4-(6-aminopyridin-3-yl)-1-methyl-1H-pyrrolo[2,3-b]pyridin-2-yl)methyl)-1,2,3,6-tetrahydropyridin-4-yl)-3,5-difluoro-N,N-dimethylbenzamide

Int. 1Z33 (0.15 g), 5-bromopyridin-2-amine (57 mg), PdDPPFCl₂-DCM (27 mg), and K₂CO₃ (0.14 g) were stirred 2 h in dioxane/H₂O (4:1, 3.5 mL) at 110° C. under MW conditions. The mixture was filtered, concentrated, and HPLC-purified to give Example 1z16 (33 mg).

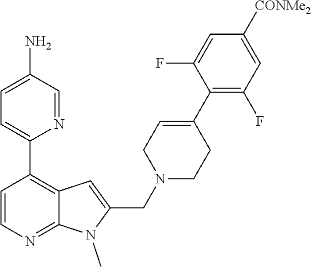

### Example 1z17. 4-(1-((4-(5-aminopyridin-2-yl)-1-methyl-1H-pyrrolo[2,3-b]pyridin-2-yl)methyl)-1,2,3,6-tetrahydropyridin-4-yl)-3,5-difluoro-N,N-dimethylbenzamide

Example 1z17 (23 mg) was prepared similarly to Example 1z16 from Int. 1Z33 (0.15 g) and 6-bromopyridin-3-amine (57 mg).

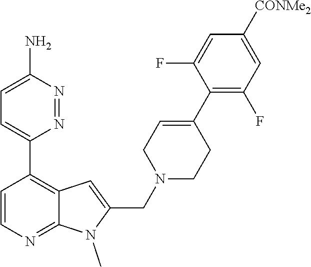

### Example 1z18. 4-(1-((4-(6-aminopyridazin-3-yl)-1-methyl-1H-pyrrolo[2,3-b]pyridin-2-yl)methyl)-1,2,3,6-tetrahydropyridin-4-yl)-3,5-difluoro-N,N-dimethylbenzamide

Example 1z18 (16 mg) was prepared similarly to Example 1z16 from Int. 1Z33 (0.10 g) and 6-bromopyridazin-3-amine (38 mg).

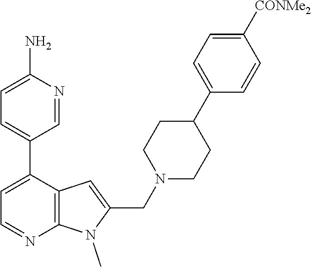

### Example 1z19. 4-(1-((4-(6-aminopyridin-3-yl)-1-methyl-1H-pyrrolo[2,3-b]pyridin-2-yl)methyl)piperidin-4-yl)-N,N-dimethylbenzamide

Example 1z19 (0.17 g) was prepared similarly to Example 1z16 from Int. 1AB1 (0.31 g) and 5-(4,4,5,5-tetramethyl-1,3,2-dioxaborolan-2-y1)pyridin-2-amine (0.18 g).

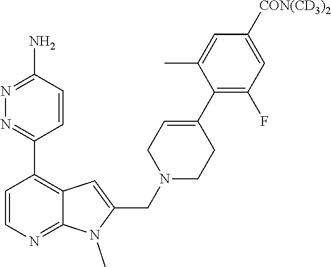

### Example 1z20. 4-(1-((4-(6-aminopyridazin-3-yl)-1-methyl-1H-pyrrolo[2,3-b]pyridin-2-yl)methyl)-1,2,3,6-tetrahydropyridin-4-yl)-3-fluoro-5-methyl-N,N-bis(methyl-d₃)benzamide

Example 1z20 (33 mg) was prepared similarly to Example 1z16 from Int. 1Z53 (0.15 g) and 6-bromopyridazin-3-amine (63 mg).

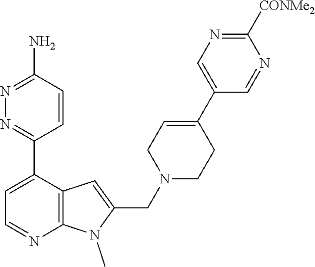

### Example 1z21. 5-(1-((4-(6-aminopyridazin-3-yl)-1-methyl-1H-pyrrolo[2,3-b]pyridin-2-yl)methyl)-1,2,3,6-tetrahydropyridin-4-yl)-N,N-dimethylpyrimidine-2-carboxamide

Example 1z21 (2 mg, HCl salt) was prepared similarly to Example 1z1 from Int. 1Z39 (75 mg).

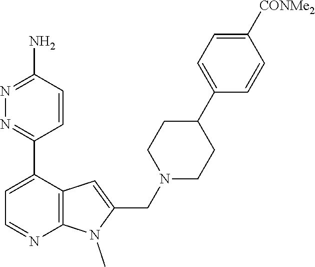

### Example 1z22. 4-(1-((4-(6-aminopyridazin-3-yl)-1-methyl-1H-pyrrolo[2,3-b]pyridin-2-yl)methyl)piperidin-4-yl)-N,N-dimethylbenzamide

Example 1z22 (5 mg, HCl salt) was prepared similarly to Example 1z1 from Int. 1Z34 (0.5 g).

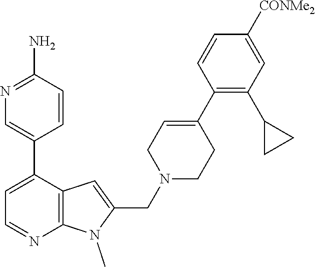

### Example 1z23. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyclopropyl-N,N-dimethylbenzamide

Example 1z23 (0.13 g) was prepared similarly to Example 1z3 from Int. 1Z109 (0.20 g) and 5-(4,4,5,5-tetramethyl-1,3,2-dioxaborolan-2-yl)pyridin-2-amine (0.10 g).

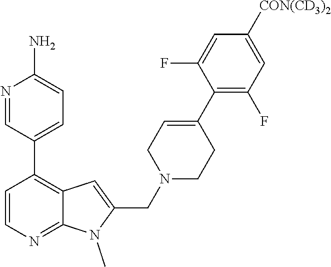

### Example 1z24. 4-(1-((4-(6-aminopyridin-3-yl)-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide

Example 1z24 (21 mg) was prepared similarly to Example 1z3 from Int. 1Z40 (0.15 g) and 5-bromopyridin-2-amine (56 mg).

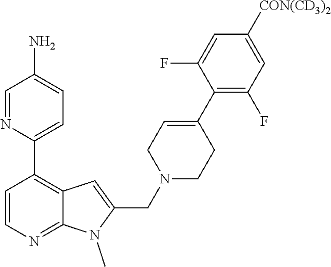

### Example 1z25. 4-(1-((4-(5-aminopyridin-2-yl)-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide

Example 1z25 (25 mg) was prepared similarly to Example 1z3 from Int. 1Z40 (0.15 g) and 6-bromopyridin-3-amine (56 mg).

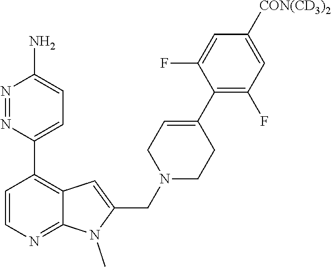

### Example 1z27. 4-(1-((4-(6-aminopyridazin-3-yl)-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide

Example 1z27 (22 mg) was prepared similarly to Example 1z3 from Int. 1Z40 (0.15 g) and 6-bromopyridazin-3-amine (57 mg).

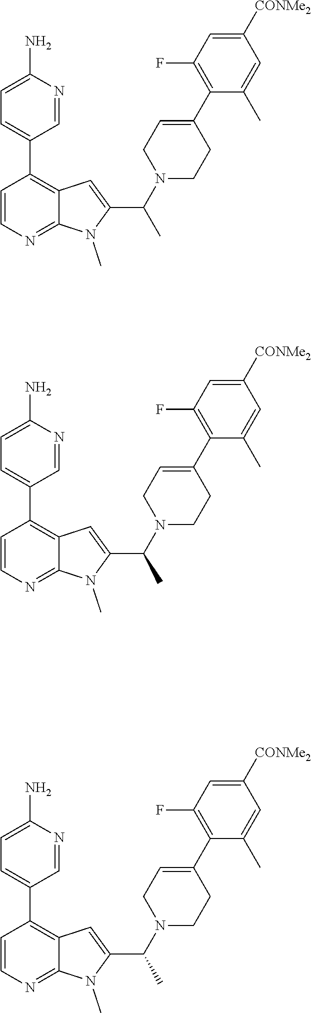

### Examples 1z28, 1z29 and 1z30. 4-(1-(1-(4-(6-aminopyridin-3-yl)-1-methyl-1H-pyrrolo[2,3-b]pyridin-2-yl)ethyl)-1,2,3,6-tetrahydropyridin-4-yl)-3-fluoro-N,N,5-trimethylbenzamide, (S)-4-(1-(1-(4-(6-aminopyridin-3-yl)-1-methyl-1H-pyrrolo[2,3-b]pyridin-2-yl)ethyl)-1,2,3,6-tetrahydropyridin-4-yl)-3-fluoro-N,N,5-trimethylbenzamide and (R)-4-(1-(1-(4-(6-aminopyridin-3-yl)-1-methyl-1H-pyrrolo[2,3-b]pyridin-2-yl)ethyl)-1,2,3,6-tetrahydropyridin-4-yl)-3-fluoro-N,N,5-trimethylbenzamide

Example 1z28 (24 mg) was prepared similarly to Example 1z3 from Int. 1Z47 (67 mg) and 5-bromopyridin-2-amine (18 mg). Example 1z28 (20 mg) was resolved by HPLC using a Chiralpak IB 250×20 mm 5 μm column with an eluent of 50% hexane containing 0.2% Et₂NH, 25% IPA containing 0.2% Et₂NH and 25% MeOH containing 0.2% Et₂NH (15 mL/min) to give Example 1z29 (9 mg, first peak) and Example 1z30 (9 mg, second peak).

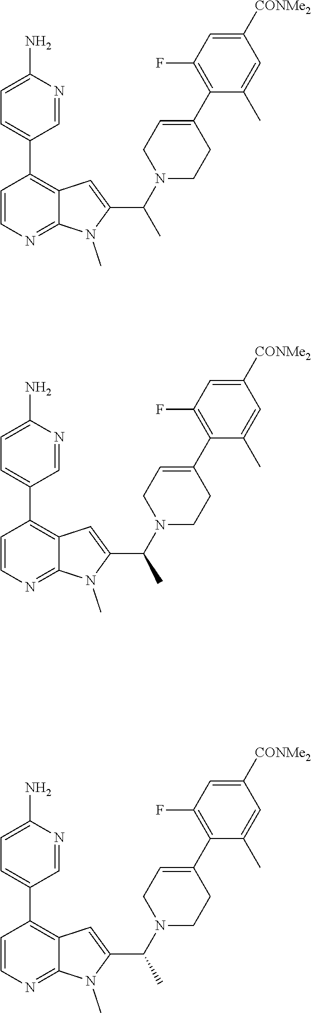

### Examples 1z31, 1z32 and 1z33. 4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-fluoro-N,N,5-trimethylbenzamide, (S)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-fluoro-N,N,5-trimethylbenzamide and (R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-fluoro-N,N,5-trimethylbenzamide

Example 1z31 (26 mg) was prepared similarly to Example 1z28 from Int. 1Z47 (67 mg) and 6-bromopyridin-3-amine (18 mg). Example 1z31 (18 mg) was resolved by HPLC using a Chiralpak IC 250×20 mm 5 μm column with an eluent of 50% IPA containing 0.2% Et₂NH and 50% MeOH (12 mL/min) to give Example 1z32 (9 mg, first peak) and Example 1z33 (8 mg, second peak).

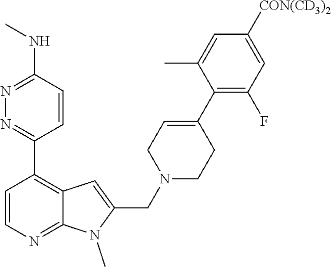

### Example 1z34. 3-fluoro-5-methyl-N,N-bis(methyl-d₃)-4-(1-((1-methyl-4-(6-(methylamino)pyridazin-3-yl)-1H-pyrrolo[2,3-b]pyridin-2-yl)methyl)-1,2,3,6-tetrahydropyridin-4-yl)benzamide

Example 1z34 (20 mg) was prepared similarly to Example 1z3 from Int. 1Z53 (0.20 g) and 6-bromo-N-methylpyridazin-3-amine (82 mg).

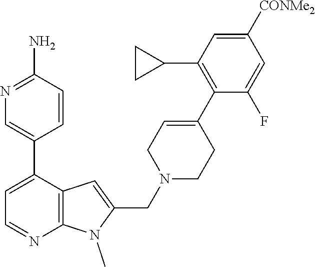

### Example 1z35. 4-(1-((4-(6-aminopyridin-3-yl)-1-methyl-1H-pyrrolo[2,3-b]pyridin-2-yl)methyl)-1,2,3,6-tetrahydropyridin-4-yl)-3-cyclopropyl-5-fluoro-N,N-dimethylbenzamide

Example 1z35 (22 mg) was prepared similarly to Example 1z3 from Int. 1Z124 (0.20 g) and 5-(4,4,5,5-tetramethyl-1,3,2-dioxaborolan-2-yl)pyridin-2-amine (0.10 g).

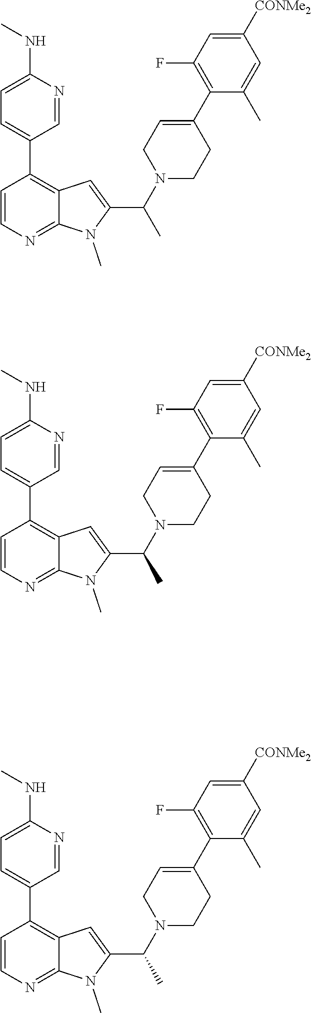

### Examples 1z36, 1z37 and 1z38. 3-fluoro-N,N,5-trimethyl-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzamide, (S)-3-fluoro-N,N,5-trimethyl-4-(1-(1-(1-methyl-4-(6-(methylamino)pyridin-3-yl)-1H-pyrrolo[2,3-b]pyridin-2-yl)ethyl)-1,2,3,6-tetrahydropyridin-4-yl)benzamide and (R)-3-fluoro-N,N,5-trimethyl-4-(1-(1-(1-methyl-4-(6-(methyl-amino)pyridin-3-yl)-1H-pyrrolo[2,3-b]pyridin-2-yl)ethyl)-1,2,3,6-tetrahydropyridin-4-yl)benzamide

Example 1z36 (15 mg) was prepared similarly to Example 1z3 from Int. 1Z47 (0.67 mg) and 5-bromo-N-methylpyridin-2-amine (20 mg). Example 1z36 (7 mg) was resolved by HPLC using a Chiralcel OD-H 250×20 mm 5 μm column with an eluent of 60% hexane containing 0.2% Et₂NH, 20% IPA containing 0.2% Et₂NH and 20% MeOH containing 0.2% Et₂NH (12 mL/min) to give Example 1z37 (3 mg, first peak) and Example 1z38 (3 mg, second peak).

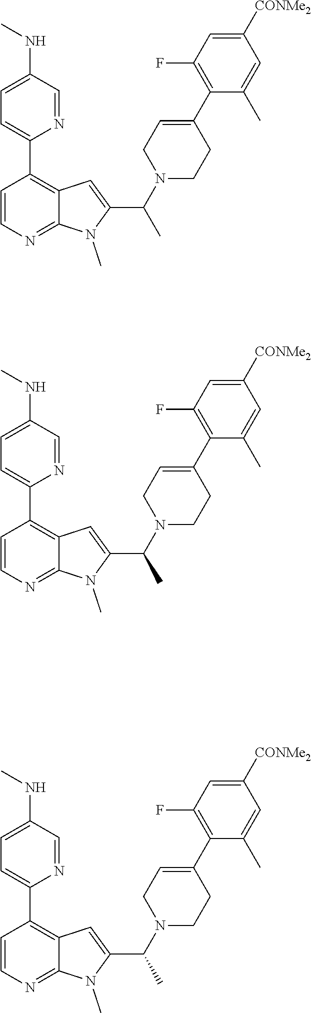

### Example 1z39, 1z40 and 1z41. 3-fluoro-N,N,5-trimethyl-4-(1-(1-(1-methyl-4-(5-(methylamino)pyridin-2-yl)-1H-pyrrolo[2,3-b]pyridin-2-yl)ethyl)-1,2,3,6-tetrahydropyridin-4-yl)benzamide, (S)-3-fluoro-N,N,5-trimethyl-4-(1-(1-(1-methyl-4-(5-(methylamino)pyridin-2-yl)-1H-pyrrolo[2,3-b]pyridin-2-yl)ethyl)-1,2,3,6-tetrahydropyridin-4-yl)benzamide and (R)-3-fluoro-N,N,5-trimethyl-4-(1-(1-(1-methyl-4-(5-(methyl-amino)pyridin-2-yl)-1H-pyrrolo[2,3-b]pyridin-2-yl)ethyl)-1,2,3,6-tetrahydropyridin-4-yl)benzamide

Example 1z39 (20 mg) was prepared similarly to Example 1z3 from Int. 1Z47 (67 mg) and 6-bromo-N-methylpyridin-3-amine (20 mg). Example 1z39 (13 mg) was resolved by HPLC using a Chiralpak IA 250×21 mm 5 μm column with an eluent of 50% hexane, 25% IPA and 25% MeOH (12 mL/min) to give Example 1z40 (6 mg, first peak) and Example 1z41 (6 mg, second peak).

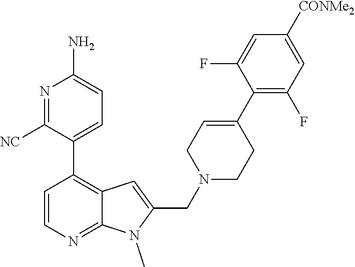

### Example 1z42. 4-(1-((4-(6-amino-2-cyanopyridin-3-yl)-1-methyl-1H-pyrrolo[2,3-b]pyridin-2-yl)methyl)-1,2,3,6-tetrahydropyridin-4-yl)-3,5-difluoro-N,N-dimethylbenzamide

Example 1z42 (52 mg) was prepared similarly to Example 1z3 from Ints. 1Z32/1Z65 (75 mg/0.15 g).

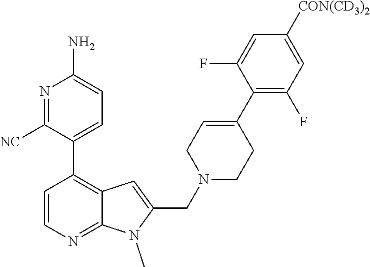

### Example 1z43. 4-(1-((4-(6-amino-2-cyanopyridin-3-yl)-1-methyl-1H-pyrrolo[2,3-b]pyridin-2-yl)methyl)-1,2,3,6-tetrahydropyridin-4-yl)-3,5-difluoro-N,N-bis(methyl-d₃)benzamide

Example 1z43 (39 mg) was prepared similarly to Example 1z3 from Ints. 1Z32/1Z92 (75 mg/0.15 g).

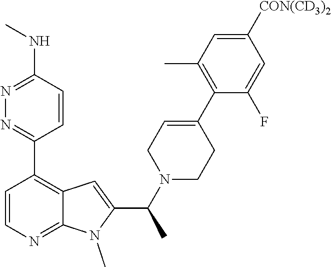

### Example 1z44. (S)-3-fluoro-5-methyl-N,N-bis(methyl-d₃)-4-(1-(1-(1-methyl-4-(6-(methylamino)-pyridazin-3-yl)-1H-pyrrolo[2,3-b]pyridin-2-yl)ethyl)-1,2,3,6-tetrahydropyridin-4-yl)benzamide

Example 1z44 (15 mg) was prepared similarly to Example 1z3 from Int. 1Z48 (0.15 g) and 6-bromo-N-methylpyridazin-3-amine (72 mg).

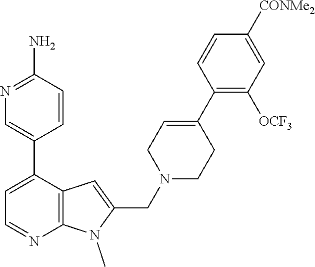

### Example 1z45. 4-(1-((4-(6-aminopyridin-3-yl)-1-methyl-1H-pyrrolo[2,3-b]pyridin-2-yl)methyl)-1,2,3,6-tetrahydropyridin-4-yl)-N,N-dimethyl-3-(trifluoromethoxy)benzamide

Int. 1Z49 (60 mg) was stirred ON in 2.8M HCl in dioxane (10 mL). The mixture was concentrated and HPLC-purified to give Example 1z45 (6 mg).

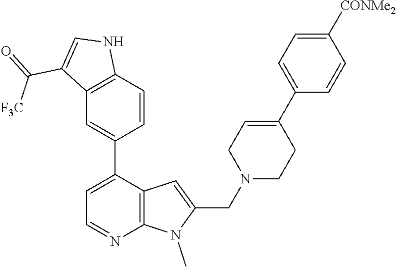

### Example 1m145. N,N-dimethyl-4-(1-((1-methyl-4-(3-(2,2,2-trifluoroacetyl)-1H-indol-5-yl)-1H-pyrrolo[2, 3-b]pyridin-2-yl)methyl)piperidin-4-yl)benzamide

Int. 1AB4 (15 mg), 1-(5-bromo-1H-indol-3-yl)-2,2,2-trifluoroethan-1-one (13 mg), K₂CO₃ (12 mg), and PdDPPFCl₂-DCM (2 mg) were stirred 1 h in DMF/H₂O (5:1, 1.2 mL) at 100° C. under an inert atmosphere. The mixture was filtered, concentrated, and HPLC-purified to give Example 1m145 (2 mg).

Pharmacology Data

| | Eotaxin-3 EC₅₀ | Eotaxin-3 | STAT6 SPR |
|---|---|---|---|
| Example | (nM) | Eₘₐₓ (%) | K_{d} (nM) |
| 1ad1 | | | 79.4 |
| 1ad2 | | | 54 |
| 1ad3 | | | 2730 |
| 1ae10 | | | 25.4 |
| 1ae14 | | | 194 |
| 1ae15 | | | 201 |
| 1ae19 | | | 628 |
| 1ae22 | 392 | | |
| 1ae4 | | | 24.5 |
| 1ae5 | | | 46.4 |
| 1ae7 | | | 67.8 |
| 1af11 | | | 102 |
| 1af12 | 246 | | 12.9 |
| 1af13 | | | 691 |
| 1af14 | 135 | | 18.5 |
| 1af15 | | | 438 |
| 1af16 | | | 15.5 |
| 1af17 | | | 6400 |
| 1af18 | 2910 | | 31.4 |
| 1af19 | | | 2360 |
| 1af20 | | 76.7 | 40.4 |
| 1af21 | | | 8660 |
| 1af22 | | | 6600 |
| 1af23 | | | 45.2 |
| 1af24 | | | 51.3 |
| 1af25 | | | 361 |
| 1af26 | 65.4 | | 20 |
| 1af27 | | | 66.5 |
| 1af28 | | | 5310 |
| 1af29 | | | 645 |
| 1af30 | | | 24.1 |
| 1af31 | | | 169 |
| 1af32 | | | 286 |
| 1af33 | | | 15.4 |
| 1af34 | | | 18.6 |
| 1af35 | | | 605 |
| 1af36 | 404 | | 15.7 |
| 1af37 | 699 | | 57.7 |
| 1af38 | | | 5270 |
| 1af39 | 115 | | 32.7 |
| 1af40 | 44400 | | 2190 |
| 1af41 | | | 3300 |
| 1af42 | 1400 | | |
| 1af43 | | | 2950 |
| 1af44 | 15900 | | 352 |
| 1af45 | 41 | | 21.6 |
| 1af46 | 760 | | 48.3 |
| 1af50 | 466 | | |
| 1af51 | 1670 | | |
| 1af52 | 713 | | |
| 1af53 | 765 | | |
| 1af54 | 534 | | |
| 1af55 | 426 | | |
| 1af56 | 726 | | |
| 1af57 | 17900 | | |
| 1af58 | 638 | | |
| 1af59 | 260 | | |
| 1af60 | 1290 | | |
| 1af64 | 3190 | | |
| 1af66 | 611 | | |
| 1af67 | 97 | | |
| 1af68 | 1090 | | |
| 1af69 | 262 | | |
| 1af7 | | | 222 |
| 1af70 | | 30-40 | |
| 1af72 | 428 | | |
| 1af73 | 46.3 | | |
| 1af75 | 159 | | |
| 1af76 | 308 | | |
| 1af77 | 871 | | |
| 1af78 | 851 | | |
| 1af79 | | 20-30 | |
| 1af8 | 369 | | 14.6 |
| 1af80 | 11.4 | | |
| 1af81 | 12.6 | | |
| 1j13 | | | 12.8 |
| 1j6 | | | 37200 |
| 1j7 | | | 3450 |
| 1k10 | | | 2630 |
| 1k11 | 6590 | | 30.5 |
| 1k12 | 15700 | | 20.1 |
| 1k13 | 14200 | | 87.7 |
| 1k14 | | 65.6 | 37.2 |
| 1k15 | | | 38.9 |
| 1k16 | | | 22.6 |
| 1k17 | | | 212 |
| 1k18 | | | 153 |
| 1k19 | | | 738 |
| 1k2 | | | 64 |
| 1k20 | | | 1510 |
| 1k21 | | | 935 |
| 1k22 | 1490 | | 22.3 |
| 1k23 | | | 25.5 |
| 1k24 | | | 336 |
| 1k25 | | | 6050 |
| 1k26 | | | 16.8 |
| 1k27 | | | 5030 |
| 1k28 | | | 428 |
| 1k3 | 6000 | | 11.4 |
| 1k30 | | | 62.1 |
| 1k31 | | | 78.5 |
| 1k32 | | | 1230 |
| 1k33 | | | 2770 |
| 1k34 | | | 245 |
| 1k35 | | | 11 |
| 1k36 | | | 1120 |
| 1k37 | 7160 | | 16 |
| 1k38 | | | 27.7 |
| 1k39 | | | 2280 |
| 1k40 | | | 475 |
| 1k41 | | | 188 |
| 1k42 | | | 731 |
| 1k43 | 871 | | 31.4 |
| 1k44 | | 77 | 25.6 |
| 1k45 | | | 27.7 |
| 1k47 | | | 1210 |
| 1k48 | | | 8230 |
| 1k49 | | | 148 |
| 1k50 | | | 557 |
| 1k51 | | 43 | 80.5 |
| 1k52 | | | 53.5 |
| 1k53 | | | 962 |
| 1k54 | | | 104 |
| 1k55 | | | 1010 |
| 1k56 | | | 1400 |
| 1k57 | | | 766 |
| 1k58 | | | 60.8 |
| 1k59 | | | 57.3 |
| 1k6 | | 57 | 9.05 |
| 1k60 | | | 35.9 |
| 1k61 | | | 16.5 |
| 1k63 | | | 16.3 |
| 1k64 | | | 1700 |
| 1k65 | | | 254 |
| 1k66 | 6620 | | 18.8 |
| 1k67 | | | 49.2 |
| 1k68 | | | 13.5 |
| 1k69 | | 72 | 53.5 |
| 1k7 | | | 835 |
| 1k70 | | | 51.9 |
| 1k71 | | 78.8 | 22.6 |
| 1k72 | | | 75.1 |
| 1k73 | 7610 | | 50.3 |
| 1k74 | | | 7290 |
| 1k75 | | | 83.9 |
| 1k76 | | | 999 |
| 1k77 | | | 52.3 |
| 1k78 | | | 59.3 |
| 1k79 | 7170 | | |
| 1k8 | | | 457 |
| 1k80 | | | 52.1 |
| 1k9 | | | 144 |
| 1m145 | | | 253 |
| 1n4 | | | 147 |
| 1n5 | | | 68.6 |
| 1n6 | | | 188 |
| 1n7 | | | 1040 |
| 1n8 | | | 2600 |
| 1n9 | | | 8920 |
| 1o1 | 410 | | 38.3 |
| 1o17 | | <20 | |
| 1o18 | | | 139 |
| 1o19 | | | 173 |
| 1o2 | 1670 | | 39.9 |
| 1o20 | | | 275 |
| 1o21 | | | 72.4 |
| 1o23 | 2460 | | 30.6 |
| 1o24 | 444 | | 31.2 |
| 1o25 | | | 1350 |
| 1o26 | 458 | | 32.4 |
| 1o27 | | | 147 |
| 1o28 | | <20 | |
| 1o29 | | <20 | |
| 1o3 | | | 249 |
| 1o4 | | | 1090 |
| 1o5 | | | 176 |
| 1o6 | 910 | | 44.7 |
| 1o7 | | 54 | 43.6 |
| 1o8 | | | 1100 |
| 1p1 | 76000 | | 329 |
| 1p10 | 239 | | |
| 1p11 | 4530 | | |
| 1p14 | 3200 | | |
| 1p15 | 1530 | | |
| 1p17 | 94 | | |
| 1p18 | 1710 | | |
| 1p2 | 14100 | | 70.1 |
| 1p20 | 214 | | |
| 1p3 | 232 | | 19.2 |
| 1p4 | 13300 | | |
| 1p5 | | | 148 |
| 1p6 | 723 | | 22 |
| 1p7 | 504 | | |
| 1p8 | | <20 | |
| 1p9 | | <20 | |
| 1q1 | 370 | | 18.1 |
| 1q2 | | | 2560 |
| 1q3 | | | 4240 |
| 1q4 | 138 | | 18.4 |
| 1q5 | 651 | | 57.4 |
| 1q6 | 3080 | | 47.4 |
| 1q7 | | | 5350 |
| 1q8 | | | 3980 |
| 1r5 | | | 110 |
| 1r6 | | 51.2 | 39.9 |
| 1s1 | | | 166 |
| 1s10 | 205 | | 40.4 |
| 1s11 | | | 133 |
| 1s12 | 4960 | | 32.1 |
| 1s13 | 480 | | 28.8 |
| 1s14 | | 37.2 | 28.9 |
| 1s15 | | | 119 |
| 1s2 | | 54.4 | 35.8 |
| 1s24 | | | 36.7 |
| 1s25 | | | 35.3 |
| 1s26 | | | 61.6 |
| 1s27 | | | 40.6 |
| 1s28 | | 76.4 | 20.6 |
| 1s3 | | | 23.2 |
| 1s30 | 2580 | | 22.1 |
| 1s4 | | | 51.1 |
| 1s5 | | | 70.6 |
| 1s6 | | | 595 |
| 1s8 | 958 | | 33.8 |
| 1s9 | | | 48.1 |
| 1u2 | 328 | | 44.9 |
| 1u3 | 104 | | 16 |
| 1u5 | | | 23.9 |
| 1x1 | 1110 | | 29.5 |
| 1x2 | | | 54.1 |
| 1x3 | | | 5280 |
| 1z1 | 5.76 | | |
| 1z10 | 473 | | |
| 1z11 | | 20-30 | |
| 1z12 | 57 | | |
| 1z13 | | | 22.3 |
| 1z14 | 37.8 | | |
| 1z15 | | <20 | |
| 1z16 | 335 | | |
| 1z17 | 368 | | |
| 1z18 | 204 | | |
| 1z19 | | | 22.3 |
| 1z20 | 53.3 | | |
| 1z21 | | 20-30 | |
| 1z22 | | <20 | |
| 1z23 | 853 | | |
| 1z28 | 21.6 | | |
| 1z29 | 1060 | | |
| 1z3 | | <20 | |
| 1z30 | 10.2 | | |
| 1z31 | 62.8 | | |
| 1z32 | 37.6 | | |
| 1z33 | 431 | | |
| 1z34 | 132 | | |
| 1z35 | 81.8 | | |
| 1z36 | 38.6 | | |
| 1z37 | 439 | | |
| 1z38 | 12.9 | | |
| 1z39 | 301 | | |
| 1z4 | | 20-30 | |
| 1z40 | 3290 | | |
| 1z41 | 110 | | |
| 1z42 | 97.3 | | |
| 1z44 | 39.5 | | |
| 1z45 | 798 | | |
| 1z5 | | 30-40 | |
| 1z6 | | <20 | |
| 1z7 | | 20-30 | |
| 1z9 | 167 | | |
| 1aa2 | | | 203 |
| 1aa2′ | | | 116 |

Eotaxin-3 EC₅₀ values provided for test compounds with >80% Eₘₐₓ
Eotaxin-3 Eₘₐₓ values provided for test compounds with <80% Eₘₐₓ
Data is provided as geometric mean values across multiple assay runs and compound batches to the extent possible
*For Example 1z42/1z43 data is provided for 1z43 (deuterium-variant of 1z42), similarly for Example 1z16/1z24 data is provided for 1z16 (hydrogen-variant of 1z24) and for Example 1z17/1z25 data is provided for 1z17 (hydrogen-variant of 1z25). The hexa-deuterio-and hydrogen-variants display similar activity as shown for example by the data for Examples 1ae10/1k66 and 1k37/1k38

### Surface Plasmon Resonance Assay

SPR was used to confirm and quantify binding of compounds to human STAT6. Biacore instruments from the 8K-series (Cytiva) were used, and measurements were performed at 25° C. Human STAT6 (truncated, aa122-658, with N-terminal His-tag, expressed in insect cells) was immobilized in the active flow cells on a CM5 chip using amine-coupling (default settings) at 10 μg/ml in 10 mM acetate buffer pH 5.5, at a contact time of 120-420 sec. and a flow rate of 10 μl/min. Reference flow cells were deactivated using blank immobilization. PBS-P+ (Cytiva) supplemented with 2% DMSO was used as running buffer, and solvent correction was applied. Compounds were tested by multi-cycle kinetics injections using a contact time of 60 sec., dissociation time of up to 300 sec., and a flow rate of 30 μl/min.

Solvent-corrected, reference-subtracted and blank-corrected data was processed and binding isotherms were fitted according to a single report point from each sensorgram (Standard: Late binding (5 sec. before injection end); alternative: Earlier report point according to binding profile). Data fitting was done both with unfixed Rmax and with Rmax fixed to the theoretical value* assuming 1:1 binding to allow processing of data from both weak and strong binders as well as to evaluate binding stoichiometry.

Positive controls were run at the beginning, during, and at the end of the screen to evaluate surface activity. Generally surface activity was only affected to a minor degree, and no corrections were applied.

$$* Equation for calculation of the theoretical R max ( Theoretical R max = ( MV Analyte / MW Ligand ) \times immobilization level ) .$$

The sensorgrams for Examples 1u2, 1x1, 1af26, 1y1, and 1af36 are shown below (where RU is plotted vs. time in seconds; dotted lines/points were excluded from the analysis due to effects from non-specific binding).

### Human Whole Blood Eotaxin-3 Assay

This was a human whole blood assay using recombinant human Interleukin 4 to stimulate the blood and measuring the ability of the test compounds to inhibit eotaxin-3 release. The biological activities of the test compounds have been tested in human whole blood stimulated with IL-4 measuring Eotaxin-3 release. The test compounds were added to 384 well clear flat-bottom plate in a 4-fold serial dilution in triplicates using a liquid handler. Human whole blood stabilized in lithium-heparin tubes was added in a volume of 65 μL per well, resulting in a final DMSO concentration of 0.5%. The plate is incubated for 2 h at 37° C. under 5% CO₂/95% air. Subsequently, 5 μL of recombinant human IL-4 (R&D Systems, cat #204-ILB) was added to the wells to a final concentration of 400 pM. The plate was incubated for 48 h at 37° C. under 5% CO₂/95% air. The plate was spun down for 10 minutes at 500×g and 20 μL supernatant was harvested and transferred to a 384 well v-bottom plate using a liquid handler and stored at −20° C. until further analysis. 10 μL supernatant was used to measure the level of Eotaxin-3 (CCL26) using Human Eotaxin-3 MSD kit from Mesocsale (Cat #K251UEK-4). The assay was performed according to the manufacture instructions.

The data from the human whole blood eotaxin-3 assay is shown below for Examples 1u2, 1x1, 1af26, and 1af36 (where the effect in % is plotted against the test concentration in M on a log-scale).

### Embodiments

Embodiment 1. a compound according to formula (I)′

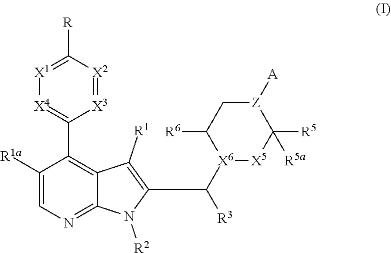

wherein A is selected from

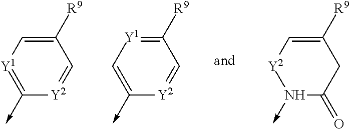

X¹ is selected from CR¹¹ and N; X² is selected from CR¹² and N; X³ is selected from CR¹³ and N, and X⁴ is selected from CR¹⁴ and N, and wherein when X⁴ is N the N may be an N-oxide;
X⁵ is CO or CR⁴;
X⁶ is N or oxidized N;
Y¹ is selected from N and CR⁷;
Y² is selected from N and CR⁸;
Z is selected from N and CR¹⁰;
R is selected from OH, CHF₂, NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′,
wherein said phenyl is optionally substituted one or more times with substituents independently selected from halogen, C₁₋₄alkyl, halo-C₁₋₄alkyl, C₃₋₄cycloalkyl, hydroxy, C₁₋₄alkoxy, cyano, —NR′R″, —CONR′R″ and —CO₂R′, wherein R′ and R″ are each independently selected from hydrogen and C₁₋₄ alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅ alkyl may optionally be substituted one or more times with fluoro;
R³ is independently selected from hydrogen and C₁₋₄alkyl;
R⁴ and R⁶ are independently selected from hydrogen and C₁₋₄alkyl and C₁₋₄alkoxy,
wherein said C₁₋₄alkyl and C₁₋₄alkoxy may optionally be substituted with one or more halogens;
R⁵ is selected from hydrogen, fluoro and C₁₋₄alkyl; wherein said C₁₋₄alkyl may optionally be substituted with one or more halogens, and R^{5a} and R¹⁰ is hydrogen; or
R⁵ and R^{5a} are both selected from fluoro or R⁵ and R^{5a} together form CO and R¹⁰ is hydrogen; or
R⁵ and R¹⁰ together form a bond, and R^{5a} is hydrogen;
R⁷ and R⁸ are independently selected from hydrogen, halogen, C₁₋₄alkyl and C₁₋₄alkoxy, wherein said C₁₋₄alkyl and C₁₋₄alkoxy may optionally be substituted with one or more halogen;
R⁹ is —CONR¹⁵R¹⁶;
R¹¹, R¹², R¹³ and R¹⁴ are independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted one or more times with a substituent independently selected from halogen, hydroxy and C₁₋₄alkoxy, wherein m is 1, 2 or 3, and wherein R′ and R″ are each independently selected from hydrogen and C₁₋₄alkyl;
R¹⁵ and R¹⁶ are independently selected from methyl and deuterated methyl;
or pharmaceutically acceptable salts thereof.

Embodiment 2. The compound according to embodiment 1 having the formula (I′)

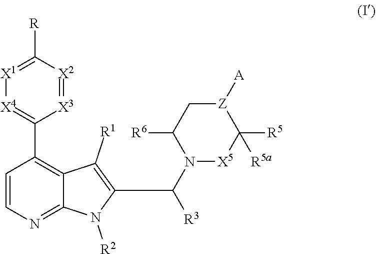

wherein A is selected from

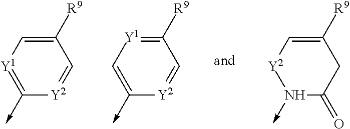

wherein X¹, X², X³, X⁴, X⁵, Y¹, Y², Z, R, R¹, R², R⁵, R^{5a}, R⁶ and R⁹ is as defined in embodiment 1 and R³ is C₁₋₄alkyl or pharmaceutically acceptable salts thereof.

Embodiment 3. A compound according to embodiment 2 having the formula (II) or (III)

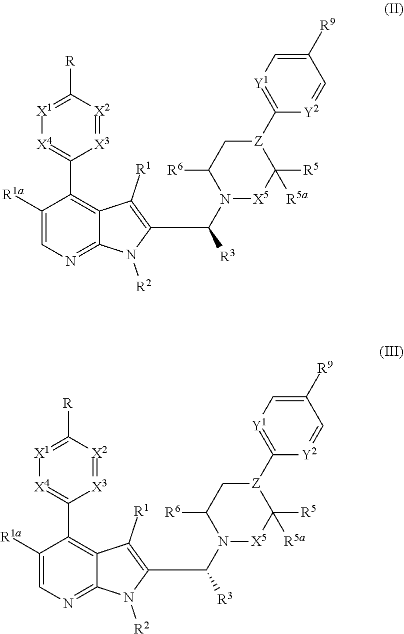

wherein X¹, X², X³, X⁴, X⁵, Y¹, Y², Z, R, R¹, R^{1a}, R², R⁵, R^{5a}, R⁶ and R⁹ is as defined in embodiment 1 and R³ is C₁₋₄alkyl, or pharmaceutically acceptable salts thereof.

Embodiment 4. A compound according to any one of embodiment 1-3, wherein X¹ is CR¹¹ and X³ is CR¹³, X⁴ is CR¹⁴ and X² is N.

Embodiment 5. A compound according to embodiment 4 wherein R¹¹, R¹³ and R¹⁴ are independently selected from hydrogen, halogen, C₁₋₄alkyl, C₁₋₄alkoxy, hydroxy, cyano, —NR′R″, —CO₂R′, tetrazole and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 6. A compound according to embodiment 5, wherein R¹¹, R¹³ and R¹⁴ are independently selected from hydrogen, methyl, methoxy, hydroxy, hydroxymethyl, fluoro, chloro, di- or trifluoromethyl, cyano, methoxycarbonyl, ethoxycarbonyl, —COOH, methylsulfonyl, tetrazolyl and (CH₃)₂N—.

Embodiment 7. The compound according to any one of embodiments 1-3, wherein X¹ is CR¹¹, X² is CR¹², X⁴ is CR¹⁴ and X³ is N.

Embodiment 8. A compound according to embodiments 7, wherein R¹¹, R¹² and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 9. The compound according to embodiment 8, wherein R¹¹, R¹² and R¹⁴ is selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 10. The compound according to any one of embodiments 1-3, wherein X¹ is CR¹¹, X² is CR¹², X³ is CR¹³ and X⁴ is CR¹⁴.

Embodiment 11. A compound according to embodiment 10, wherein R¹¹, R¹², R¹³ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 12. The compound according to embodiment 11, wherein R¹¹, R¹², R¹³ and R¹⁴ is selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 13. The compound according to any one of embodiments 1-3, wherein X¹ is N; X² is selected from CR¹², X³ is N and X⁴ is selected from CR¹⁴.

Embodiment 14. A compound according to embodiment 13, wherein R¹² and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 15. The compound according to embodiment 14, wherein R¹² and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 16. The compound according to any one of embodiments 1-3, wherein X¹ is N, X² is CR¹²; X³ is CR¹³ and X⁴ is CR¹⁴.

Embodiment 17. A compound according to embodiment 16, wherein R¹³ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 18. The compound according to embodiment 17, wherein R¹³ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 19. The compound according to any one of embodiments 1-3, wherein X¹ is CR¹¹, X² is N; X³ is N and X⁴ is CR¹⁴.

Embodiment 20. A compound according to embodiment 19, wherein R¹¹ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 21. The compound according to embodiment 20, wherein R¹¹ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 22. The compound according to any one of embodiments 1-3, wherein X¹, X⁴, X³ is N and X² is CR¹².

Embodiment 23. A compound according to embodiment 22, wherein R¹² is selected from hydrogen, halogen, C₁₋₄alkyl, C₁₋₄alkoxy, hydroxy, cyano, —NR′R″, —CO₂R′, tetrazolyl and —SO₂—C₁₋₄ alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 24. The compound according to embodiment 23, wherein R¹² is selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 25. The compound according to any one of embodiments 1-3, wherein X¹, X² is N, X³ is N and X⁴ is CR¹⁴.

Embodiment 26. A compound according to embodiment 25, wherein R¹³ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 27. The compound according to embodiment 26, wherein R¹³ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 28. The compound according to any one of embodiments 1-3, wherein X³ and X⁴ is N and X¹ is CR¹¹ and X² is CR¹².

Embodiment 29. A compound according to embodiment 28, wherein R¹¹ and R¹⁴ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 30. The compound according to embodiment 29, wherein R¹¹ and R¹⁴ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 31. The compound according to any one of embodiments 1-3, wherein X¹ is CR¹¹, X² is CR¹², X³ is CR¹³, X⁴ is N⁺—O⁻.

Embodiment 32. A compound according to embodiment 31, wherein R¹¹, R¹² and R¹³ are independently selected from hydrogen, halogen, C₁₋₄alkyl, C₁₋₄alkoxy, hydroxy, cyano, —NR′R″, —CO₂R′, tetrazolyl and —SO₂—C₁₋₄alkyl, wherein said C₁₋₄alkyl is optionally substituted one or more times with a substituent independently selected from halogen, hydroxy and C₁₋₄alkoxy and wherein R′ and R″ are each independently selected from hydrogen and C₁₋₄alkyl.

Embodiment 33. The compound according to embodiment 32, wherein R¹¹, R¹² and R¹³ are independently selected from hydrogen, methyl, methoxy, hydroxy, hydroxymethyl, fluoro, chloro, di- or trifluoromethyl, cyano, methoxycarbonyl, —COOH, ethoxycarbonyl, methylsulfonyl, tetrazolyl and (CH₃)₂N—.

Embodiment 34. The compound according to any one of embodiments 1-33, wherein Z is N.

Embodiment 35. The compound according to any one of embodiments 1-33, wherein Z is CR¹⁰, and R⁵ and R¹⁰ together form a bond.

Embodiment 36. The compound according to any one of embodiments 1-33, wherein Z is CR¹⁰ and R¹⁰ is hydrogen.

Embodiment 37. The compound according to any one of embodiments 1-36, wherein R^{5a} is hydrogen.

Embodiment 38. The compound according to any one of embodiments 1-37, wherein Y¹ is CR⁷ and Y² is CR⁸.

Embodiment 39. The compound according to any one of embodiments 1-37, wherein Y¹ is CR⁷ and Y² is N.

Embodiment 40. The compound according to any one of embodiments 1-37, wherein Y¹ and Y² is N.

Embodiment 41. The compound according to embodiment 38, wherein
(a) both of R⁷ and R⁸ is C₁₋₄alkyl;
(b) both of R⁷ and R⁸ is halogen;
(c) one of R⁷ and R⁸ is C₁₋₄alkyl and the other is halogen;
(d) one of R⁷ and R⁸ is C₁₋₄alkyl and the other is hydrogen; or
(e) one of R⁷ and R⁸ is halogen and the other is hydrogen.

Embodiment 42. A compound according to embodiment 38, wherein one or both of R⁷ and R⁸ is hydrogen.

Embodiment 43. A compound according to embodiment 38, wherein one of R⁷ and R⁸ is hydrogen and the other is fluoro.

Embodiment 44. The compound according to any one of embodiments 1-43, wherein R is selected from —NH₂, —NHCH₃ and —NH(CH₂)₂OH.

Embodiment 45. A compound according to any one of embodiments 1-44 wherein R⁹ is selected from CON(CH₃)₂ and —CON(CD₃)₂.

Embodiment 46. A compound according to any one of embodiments 1-45 wherein R¹ is hydrogen, R² is hydrogen and R³ is selected from hydrogen and methyl.

Embodiment 46a. A compound selected from
N,N-Dimethyl-4-[(3S,4R)-1-[[4-(4-acetamidophenyl)-1-methyl-pyrrolo[2,3-b]pyridin-2-yl]methyl]-3-methyl-2-oxo-4-piperidyl]benzamide;
N,N-dimethyl-4-[(3R,4S)-1-[[4-(4-acetamidophenyl)-1-methyl-pyrrolo[2,3-b]pyridin-2-yl]methyl]-3-methyl-2-oxo-4-piperidyl]benzamide;
4-(1-((4-(5-Amino-6-hydroxypyridin-2-yl)-1-methyl-1H-pyrrolo[2,3-b]pyridin-2-yl)methyl)piperidin-4-yl)-N,N-dimethylbenzamide;
4-(1-((4-(5-Amino-6-methoxypyridin-2-yl)-1-methyl-1H-pyrrolo[2,3-b]pyridin-2-yl)methyl)piperidin-4-yl)-N,N-dimethylbenzamide;
4-[1-[[1-iso-Propyl-4-[6-(methylamino)-3-pyridyl]pyrrolo[2,3-b]pyridin-2-yl]methyl]-4-piperidyl]-N,N-dimethyl-benzamide;
R)—N,N-Dimethyl-4-[1-[[4-(6-amino-3-pyridyl)-1-methyl-pyrrolo[2,3-b]pyridin-2-yl]methyl]-2-oxo-4-piperidyl]benzamide;
(S)—N,N-dimethyl-4-[1-[[4-(6-amino-3-pyridyl)-1-methyl-pyrrolo[2,3-b]pyridin-2-yl]methyl]-2-oxo-4-piperidyl]benzamide
4-[4-[[4-(6-Amino-3-pyridyl)-1-methyl-pyrrolo[2,3-b]pyridin-2-yl]methyl]-3-oxo-piperazin-1-yl]-N,N-dimethyl-benzamide
4-[1-[[4-(5-Amino-2-pyridyl)-1-methyl-pyrrolo[2,3-b]pyridin-2-yl]methyl]-4-piperidyl]-N,N-bis(trideuteriomethyl)benzamide;
4-[1-[[4-(6-Amino-2-cyano-3-pyridyl)-1-methyl-pyrrolo[2,3-b]pyridin-2-yl]methyl]-4-piperidyl]-N-methyl-benzamide;
4-[1-[[4-(6-Amino-2-cyano-3-pyridyl)-1-methyl-pyrrolo[2,3-b]pyridin-2-yl]methyl]-4-piperidyl]-N-(trideuteriomethyl)benzamide yl]methyl]-2-oxo-4-piperidyl]benzamide;
4-[1-[[1-(2,2-Difluoropropyl)-4-[6-(methylamino)-3-pyridyl]pyrrolo[2,3-b]pyridin-2-yl]methyl]-4-piperidyl]-N,N-dimethyl-benzamide;
1′-((4-(6-Aminopyridazin-3-yl)-1-methyl-1H-pyrrolo[2,3-b]pyridin-2-yl)methyl)-N,N,3-trimethyl-1′,2′,3′,6′-tetrahydro-[2,4′-bipyridine]-5-carboxamide
(S)—N,N-Dimethyl-4-(1-(1-(1-methyl-4-(6-(methylamino)pyridin-3-yl)-1H-pyrrolo[2,3-b]pyridin-2-yl)ethyl)piperidin-4-yl)benzamide;
(R)—N,N-Dimethyl-4-(1-(1-(1-methyl-4-(6-(methylamino)pyridin-3-yl)-1H-pyrrolo[2,3-b]pyridin-2-yl)ethyl)piperidin-4-yl)benzamide;
(S)-4-(1-(1-(4-(6-Aminopyridin-3-yl)-1-methyl-1H-pyrrolo[2,3-b]pyridin-2-yl)ethyl)-piperidin-4-yl)-N,N-dimethylbenzamide;
(R)-4-(1-(1-(4-(6-aminopyridin-3-yl)-1-methyl-1H-pyrrolo[2,3-b]pyridin-2-yl)ethyl)piperidin-4-yl)-N,N-dimethylbenzamide;
(S)-4-(1-(1-(4-(6-Amino-2-cyanopyridin-3-yl)-1-methyl-1H-pyrrolo[2,3-b]pyridin-2-yl)ethyl)piperidin-4-yl)-N,N-dimethylbenzamide;
(R)-4-(1-(1-(4-(6-amino-2-cyanopyridin-3-yl)-1-methyl-1H-pyrrolo[2,3-b]pyridin-2-yl)ethyl)piperidin-4-yl)-N,N-dimethylbenzamide;
(S)-4-(1-(1-(4-(5-Aminopyridin-2-yl)-1-methyl-1H-pyrrolo[2,3-b]pyridin-2-yl)-ethyl)piperidin-4-yl)-N,N-dimethylbenzamide;
(R)-4-(1-(1-(4-(5-aminopyridin-2-yl)-1-methyl-1H-pyrrolo[2,3-b]pyridin-2-yl)ethyl)piperidin-4-yl)-N,N-dimethylbenzamide;
(S)-4-(1-(1-(4-(5-Aminopyridin-2-yl)-1-methyl-1H-pyrrolo[2,3-b]pyridin-2-yl)ethyl)piperidin-4-yl)-N,N,3-trimethylbenzamide;
(R)-4-(1-(1-(4-(5-aminopyridin-2-yl)-1-methyl-1H-pyrrolo[2,3-b]pyridin-2-yl)ethyl)piperidin-4-yl)-N,N,3-trimethylbenzamide;
(S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-dimethylbenzamide;
(R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N-dimethylbenzamide;
(S)-4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide;
(R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N-dimethylbenzamide;
(R)-4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide;
(R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide;
(S)-4-(1-(1-(4-(6-Amino-2-cyanopyridin-3-yl)-1-methyl-1H-pyrrolo[2,3-b]pyridin-2-yl)ethyl)piperidin-4-yl)-N,N,3-trimethylbenzamide;
(R)-4-(1-(1-(4-(6-amino-2-cyanopyridin-3-yl)-1-methyl-1H-pyrrolo[2,3-b]pyridin-2-yl)ethyl)piperidin-4-yl)-N,N,3-trimethylbenzamide;
(S)-4-(1-(1-(4-(6-Amino-2-cyanopyridin-3-yl)-1-methyl-1H-pyrrolo[2,3-b]pyridin-2-yl)ethyl)piperidin-4-yl)-N,N-bis(methyl-d₃)benzamide;
(R)-4-(1-(1-(4-(6-amino-2-cyanopyridin-3-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide;
(R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N,3-trimethylbenzamide;
(R)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide;
(S)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-3-methyl-N,N-bis(methyl-d₃)benzamide;
(S)-4-(1-(1-(4-(5-Aminopyridin-2-yl)-1-methyl-1H-pyrrolo[2,3-b]pyridin-2-yl)ethyl)piperidin-4-yl)-N,N-bis(methyl-d₃)benzamide;
(R)-4-(1-(1-(4-(5-aminopyridin-2-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(6-Amino-2-cyanopyridin-3-yl)-1-methyl-1H-pyrrolo[2,3-b]pyridin-2-yl)ethyl)piperidin-4-yl)-3-methyl-N,N-bis(methyl-d₃)benzamide;
(R)-4-(1-(1-(4-(6-amino-2-cyanopyridin-3-yl)-1-methyl-1H-pyrrolo[2,3-b]pyridin-2-yl)ethyl)piperidin-4-yl)-3-methyl-N,N-bis(methyl-d₃)benzamide
(S)-4-(1-(1-(4-(2-Cyano-6-(methylamino)pyridin-3-yl)-1-methyl-1H-pyrrolo[2,3-b]pyridin-2-yl)ethyl)piperidin-4-yl)-N,N-dimethylbenzamide;
(R)-4-(1-(1-(4-(2-cyano-6-(methyl-amino)pyridin-3-yl)-1-methyl-1H-pyrrolo[2,3-b]pyridin-2-yl)ethyl)piperidin-4-yl)-N,N-dimethylbenzamide;
(S)-4-(1-(1-(4-(5-Aminopyridin-2-yl)-1-methyl-1H-pyrrolo[2,3-b]pyridin-2-yl)ethyl)piperidin-4-yl)-3-methyl-N,N-bis(methyl-d₃)benzamide;
(R)-4-(1-(1-(4-(5-aminopyridin-2-yl)-1-methyl-1H-pyrrolo[2,3-b]pyridin-2-yl)ethyl)piperidin-4-yl)-3-methyl-N,N-bis(methyl-d₃)benzamide;
(S)-4-(1-(1-(4-(5-Aminopyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(R)-4-(1-(1-(4-(5-aminopyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-Amino-2-cyanopyridin-3-yl)-1-methyl-1H-pyrrolo[2,3-b]pyridin-2-yl)ethyl)-1,2,3,6-tetrahydropyridin-4-yl)-N,N,3-trimethylbenzamide;
(R)-4-(1-(1-(4-(6-amino-2-cyanopyridin-3-yl)-1-methyl-1H-pyrrolo[2,3-b]pyridin-2-yl)ethyl)-1,2,3,6-tetrahydropyridin-4-yl)-N,N,3-trimethylbenzamide;
(S)-4-(1-(1-(4-(6-((2-methoxyethyl)amino)pyridin-3-yl)-1-methyl-1H-pyrrolo[2,3-b]pyridin-2-yl)ethyl)piperidin-4-yl)-N,N-bis(methyl-d₃)benzamide
(S)-4-(1-(1-(4-(5-Amino-3-fluoropyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(2-Amino-4-cyanopyrimidin-5-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(5-Amino-3-methylpyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(5-Amino-6-fluoropyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-Amino-4-fluoropyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-Amino-5-fluoropyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(5-Amino-3-(trifluoromethyl)pyridin-2-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-Amino-5-methylpyridin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-N,N-bis(methyl-d₃)benzamide;
(S)—N,N-bis(methyl-d₃)-4-(1-(1-(1-methyl-4-(6-(methylamino)pyridazin-3-yl)-1H-(S)-4-(1-(1-(4-(3-amino-1,2,4-triazin-6-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(5-amino-6-methylpyridin-2-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(6-amino-2-methoxypyridin-3-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(5-amino-3-methoxypyridin-2-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(6-((2-hydroxyethyl)amino)pyridin-3-yl)-1-methyl-1H-pyrrolo[2,3-b]pyridin-2-yl)ethyl)piperidin-4-yl)-N,N-bis(methyl-d₃)benzamide;
(S)-4-(1-(1-(4-(6-amino-4-cyanopyridin-3-yl)-1-methyl-1H-pyrrolo[2,3-b]pyridin-2-yl)ethyl)piperidin-4-yl)-N,N-bis(methyl-d₃)benzamide;
4-((2R)-1-((4-(6-acetamidopyridin-3-yl)-1-methyl-1H-pyrrolo[2,3-b]pyridin-2-yl)methyl)-2-methylpiperidin-4-yl)-N,N-bis(methyl-d₃)benzamide;
2-(1-((4-(6-Aminopyridin-3-yl)-1-methyl-1H-pyrrolo[2,3-b]pyridin-2-yl)methyl)-1,2,3,6-tetrahydropyridin-4-yl)-N,N-bis(methyl-d₃)pyrimidine-5-carboxamide;
4-(1-((4-(6-Aminopyridazin-3-yl)-1-methyl-1H-pyrrolo[2,3-b]pyridin-2-yl)methyl)-1,2,3,6-tetrahydropyridin-4-yl)-3-chloro-5-fluoro-N,N-dimethylbenzamide;
4-(1-((4-(6-Aminopyridazin-3-yl)-1-methyl-1H-pyrrolo[2,3-b]pyridin-2-yl)methyl)-1,2,3,6-tetrahydropyridin-4-yl)-N,N,3-trimethylbenzamide;
4-(1-((4-(6-Aminopyridazin-3-yl)-1-methyl-1H-pyrrolo[2,3-b]pyridin-2-yl)methyl)-1,2,3,6-tetrahydropyridin-4-yl)-3-chloro-N,N-dimethylbenzamide;
1′-((4-(6-Aminopyridazin-3-yl)-1-methyl-1H-pyrrolo[2,3-b]pyridin-2-yl)methyl)-N,N-dimethyl-1′,2′,3′,6′-tetrahydro-[2,4′-bipyridine]-5-carboxamide;
5-(1-((4-(6-Aminopyridin-3-yl)-1-methyl-1H-pyrrolo[2,3-b]pyridin-2-yl)methyl)-1,2,3,6-tetrahydropyridin-4-yl)-N,N-bis(methyl-d3)pyrazine-2-carboxamide;
(S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-3-fluoro-N,N,5-trimethylbenzamide;
(S)-4-(1-(1-(4-(6-aminopyridazin-3-yl)-1-methyl-1H-pyrrolo[2,3-b]pyridin-2-yl)ethyl)-1,2,3,6-tetrahydropyridin-4-yl)-3-fluoro-5-methyl-N,N-bis(methyl-d₃)benzamide;
4-[2-[[4-[4-(Dimethylcarbamoyl)phenyl]piperazin-1-yl]methyl]-1-methyl-pyrrolo[2,3-b]pyridin-4-yl]-N-methyl-benzamide;
4-[2-[[4-[4-(Dimethylcarbamoyl)phenyl]-1-piperidyl]methyl]-1-methyl-pyrrolo[2,3-b]pyridin-4-yl]-N-methyl-benzamide;
2-(2-Amino-5-(2-((4-(4-(dimethylcarbamoyl)phenyl)piperidin-1-yl)methyl)-1-methyl-1H-pyrrolo[2,3-b]pyridin-4-yl)phenyl)-2-oxoacetic acid;
4-[1-[[4-(6-Acetamido-3-pyridyl)-1-methyl-pyrrolo[2,3-b]pyridin-2-yl]methyl]-4-piperidyl]-N,N-dimethyl-benzamide;
4-[1-[[4-(6-Amino-3-pyridyl)-1-methyl-pyrrolo[2,3-b]pyridin-2-yl]methyl]-4-piperidyl]-N,N-dimethyl-benzamide;
2-Amino-5-[2-[[4-[4-(dimethylcarbamoyl)phenyl]-1-piperidyl]methyl]-1-methyl-pyrrolo[2,3-b]pyridin-4-yl]benzoic acid;
4-(1-((4-(6-Amino-5-methoxypyridin-3-yl)-1-methyl-1H-pyrrolo[2,3-b]pyridin-2-yl)methyl)piperidin-4-yl)-N,N-dimethylbenzamide;
4-[1-[[4-(5-Aminopyrazin-2-yl)-1-methyl-pyrrolo[2,3-b]pyridin-2-yl]methyl]-4-piperidyl]-N,N-dimethyl-benzamide;
2-Amino-5-[2-[[4-[4-(dimethylcarbamoyl)phenyl]-1-piperidyl]methyl]-1-methyl-pyrrolo[2,3-b]pyridin-4-yl]pyridine-3-carboxylic acid;
Methyl 2-amino-5-[2-[[4-[4-(dimethylcarbamoyl)phenyl]-1-piperidyl]methyl]-1-methyl-pyrrolo[2,3-b]pyridin-4-yl]pyridine-3-carboxylate;
4-[1-[[4-(6-Amino-5-methyl-3-pyridyl)-1-methyl-pyrrolo[2,3-b]pyridin-2-yl]methyl]-4-piperidyl]-N,N-dimethyl-benzamide;
4-[1-[[4-(6-Amino-2-methyl-3-pyridyl)-1-methyl-pyrrolo[2,3-b]pyridin-2-yl]methyl]-4-piperidyl]-N,N-dimethyl-benzamide;
4-[1-[[4-(2-Aminopyrimidin-5-yl)-1-methyl-pyrrolo[2,3-b]pyridin-2-yl]methyl]-4-piperidyl]-N,N-dimethyl-benzamide;
4-[1-[[4-(6-Amino-5-fluoro-3-pyridyl)-1-methyl-pyrrolo[2,3-b]pyridin-2-yl]methyl]-4-piperidyl]-N,N-dimethyl-benzamide;
4-[1-[[4-(6-Amino-4-fluoro-3-pyridyl)-1-methyl-pyrrolo[2,3-b]pyridin-2-yl]methyl]-4-piperidyl]-N,N-dimethyl-benzamide;
4-[1-[[4-(6-Amino-4-fluoro-3-pyridyl)-1-methyl-pyrrolo[2,3-b]pyridin-2-yl]methyl]-4-piperidyl]-N,N-bis(trideuteriomethyl)benzamide;
4-[1-[[4-[6-Amino-2-(trifluoromethyl)-3-pyridyl]-1-methyl-pyrrolo[2,3-b]pyridin-2-yl]methyl]-4-piperidyl]-N,N-dimethyl-benzamide;
4-[1-[[4-[6-Amino-2-(trifluoromethoxy)-3-pyridyl]-1-methyl-pyrrolo[2,3-b]pyridin-2-yl]methyl]-4-piperidyl]-N,N-dimethyl-benzamide;
4-[1-[[4-(6-Amino-5-methoxy-4-methyl-3-pyridyl)-1-methyl-pyrrolo[2,3-b]pyridin-2-yl]methyl]-4-piperidyl]-N,N-dimethyl-benzamide;
4-[1-[[4-(5-Amino-6-methyl-pyrazin-2-yl)-1-methyl-pyrrolo[2,3-b]pyridin-2-yl]methyl]-4-piperidyl]-N,N-dimethyl-benzamide;
4-[1-[[4-(6-Amino-5-cyano-3-pyridyl)-1-methyl-pyrrolo[2,3-b]pyridin-2-yl]methyl]-4-piperidyl]-N,N-dimethyl-benzamide;
4-[1-[[4-(6-Amino-2-methoxy-3-pyridyl)-1-methyl-pyrrolo[2,3-b]pyridin-2-yl]methyl]-4-piperidyl]-N,N-dimethyl-benzamide;
4-[1-[[4-(6-Amino-2-methoxy-3-pyridyl)-1-methyl-pyrrolo[2,3-b]pyridin-2-yl]methyl]-4-piperidyl]-N,N-bis(trideuteriomethyl)benzamide;
4-[1-[[4-[6-Amino-5-(difluoromethyl)-3-pyridyl]-1-methyl-pyrrolo[2,3-b]pyridin-2-yl]methyl]-4-piperidyl]-N,N-dimethyl-benzamide;
4-[1-[[4-(6-Amino-5-methylsulfonyl-3-pyridyl)-1-methyl-pyrrolo[2,3-b]pyridin-2-yl]methyl]-4-piperidyl]-N,N-dimethyl-benzamide;
Methyl 2-[[5-[2-[[4-[4-(dimethylcarbamoyl)phenyl]-1-piperidyl]methyl]-1-methyl-pyrrolo[2,3-b]pyridin-4-yl]-2-pyridyl]amino]acetate;
4-[1-[[4-[6-Amino-4-(trifluoromethyl)-3-pyridyl]-1-methyl-pyrrolo[2,3-b]pyridin-2-yl]methyl]-4-piperidyl]-N,N-dimethyl-benzamide;
4-[1-[[4-(6-Acetamido-4-methyl-3-pyridyl)-1-methyl-pyrrolo[2,3-b]pyridin-2-yl]methyl]-4-piperidyl]-N,N-dimethyl-benzamide;
4-[1-[[4-(6-Amino-4-methyl-3-pyridyl)-1-methyl-pyrrolo[2,3-b]pyridin-2-yl]methyl]-4-piperidyl]-N,N-dimethyl-benzamide;
4-[1-[[4-(6-Amino-2,5-dimethyl-3-pyridyl)-1-methyl-pyrrolo[2,3-b]pyridin-2-yl]methyl]-4-piperidyl]-N,N-dimethyl-benzamide;
4-[1-[[4-(4-Amino-2,6-dimethyl-phenyl)-1-methyl-pyrrolo[2,3-b]pyridin-2-yl]methyl]-4-piperidyl]-N,N-dimethyl-benzamide;
4-[1-[[4-(6-Amino-5-methoxy-2-methyl-3-pyridyl)-1-methyl-pyrrolo[2,3-b]pyridin-2-yl]methyl]-4-piperidyl]-N,N-dimethyl-benzamide;
4-[1-[[4-[6-Amino-5-(hydroxymethyl)-3-pyridyl]-1-methyl-pyrrolo[2,3-b]pyridin-2-yl]methyl]-4-piperidyl]-N,N-dimethyl-benzamide;
Ethyl 6-amino-3-[2-[[4-[4-(dimethylcarbamoyl)phenyl]-1-piperidyl]methyl]-1-methyl-pyrrolo[2,3-b]pyridin-4-yl]pyridine-2-carboxylate;
4-[1-[[4-[6-Amino-5-(trifluoromethyl)-3-pyridyl]-1-methyl-pyrrolo[2,3-b]pyridin-2-yl]methyl]-4-piperidyl]-N,N-dimethyl-benzamide;
N,N-Dimethyl-4-[1-[[1-methyl-4-[6-(methylamino)-3-pyridyl]pyrrolo[2,3-b]pyridin-2-yl]methyl]-4-piperidyl]benzamide
4-[1-[[1-Methyl-4-[6-(methylamino)-3-pyridyl]pyrrolo[2,3-b]pyridin-2-yl]methyl]-4-piperidyl]-N,N-bis(trideuteriomethyl)benzamide;
4-[1-[[4-(5-Amino-3-methyl-pyrazin-2-yl)-1-methyl-pyrrolo[2,3-b]pyridin-2-yl]methyl]-4-piperidyl]-N,N-dimethyl-benzamide;
4-[1-[[4-(6-Acetamido-2-methyl-3-pyridyl)-1-methyl-pyrrolo[2,3-b]pyridin-2-yl]methyl]-4-piperidyl]-N,N-dimethyl-benzamide;
4-[1-[[4-(2-Amino-4-cyano-pyrimidin-5-yl)-1-methyl-pyrrolo[2,3-b]pyridin-2-yl]methyl]-4-piperidyl]-N,N-dimethyl-benzamide;
4-[1-[[4-(6-Amino-5-cyano-4-methyl-3-pyridyl)-1-methyl-pyrrolo[2,3-b]pyridin-2-yl]methyl]-4-piperidyl]-N,N-dimethyl-benzamide;
4-[1-[[4-(6-Amino-2-cyano-3-pyridyl)-1-methyl-pyrrolo[2,3-b]pyridin-2-yl]methyl]-4-piperidyl]-N,N-dimethyl-benzamide;
4-[1-[[4-(6-Amino-2-methoxy-3-pyridyl)-1-methyl-pyrrolo[2,3-b]pyridin-2-yl]methyl]-4-piperidyl]-N,N-bis(trideuteriomethyl)benzamide;
4-[1-[[4-(6-Amino-2-cyano-3-pyridyl)-1-(trideuteriomethyl)pyrrolo[2,3-b]pyridin-2-yl]methyl]-4-piperidyl]-N,N-dimethyl-benzamide;
4-[1-[[4-[6-Amino-5-(dimethylamino)-3-pyridyl]-1-methyl-pyrrolo[2,3-b]pyridin-2-yl]methyl]-4-piperidyl]-N,N-dimethyl-benzamide;
4-[1-[[4-[6-Amino-5-(trifluoromethoxy)-3-pyridyl]-1-methyl-pyrrolo[2,3-b]pyridin-2-yl]methyl]-4-piperidyl]-N,N-dimethyl-benzamide;
4-[1-[[4-[6-Amino-4-(hydroxymethyl)-3-pyridyl]-1-methyl-pyrrolo[2,3-b]pyridin-2-yl]methyl]-4-piperidyl]-N,N-dimethyl-benzamide;
4-[1-[[4-(6-Amino-5-hydroxy-3-pyridyl)-1-methyl-pyrrolo[2,3-b]pyridin-2-yl]methyl]-4-piperidyl]-N,N-dimethyl-benzamide;
N,N-Dimethyl-4-[1-[[1-methyl-4-[4-methyl-6-(methylamino)-3-pyridyl]pyrrolo[2,3-b]pyridin-2-yl]methyl]-4-piperidyl]benzamide;
4-[1-[[4-[6-Amino-4-(difluoromethyl)-3-pyridyl]-1-methyl-pyrrolo[2,3-b]pyridin-2-yl]methyl]-4-piperidyl]-N,N-dimethyl-benzamide;
4-[1-[[4-(6-Amino-4-cyano-3-pyridyl)-1-methyl-pyrrolo[2,3-b]pyridin-2-yl]methyl]-4-piperidyl]-N,N-dimethyl-benzamide;
4-[1-[[4-(3-Amino-1,2,4-triazin-6-yl)-1-methyl-pyrrolo[2,3-b]pyridin-2-yl]methyl]-4-piperidyl]-N,N-dimethyl-benzamide;
4-[1-[[4-[4-Amino-2,6-bis(difluoromethyl)phenyl]-1-methyl-pyrrolo[2,3-b]pyridin-2-yl]methyl]-4-piperidyl]-N,N-dimethyl-benzamide;
Methyl 2-amino-5-[2-[[4-[4-(dimethylcarbamoyl)phenyl]-1-piperidyl]methyl]-1-methyl-pyrrolo[2,3-b]pyridin-4-yl]pyridine-4-carboxylate;
4-[1-[[4-(5-Amino-6-cyano-pyrazin-2-yl)-1-methyl-pyrrolo[2,3-b]pyridin-2-yl]methyl]-4-piperidyl]-N,N-dimethyl-benzamide;
4-[1-[[4-[6-Amino-2-(hydroxymethyl)-3-pyridyl]-1-methyl-pyrrolo[2,3-b]pyridin-2-yl]methyl]-4-piperidyl]-N,N-dimethyl-benzamide;
4-[1-[[4-[6-(2-Methoxyethylamino)-3-pyridyl]-1-methyl-pyrrolo[2,3-b]pyridin-2-yl]methyl]-4-piperidyl]-N,N-dimethyl-benzamide;
4-[1-[[4-[4-Amino-3-(2H-tetrazol-5-yl)phenyl]-1-methyl-pyrrolo[2,3-b]pyridin-2-yl]methyl]-4-piperidyl]-N,N-dimethyl-benzamide;
4-[1-[[4-[6-(2-Hydroxyethylamino)-3-pyridyl]-1-methyl-pyrrolo[2,3-b]pyridin-2-yl]methyl]-4-piperidyl]-N,N-dimethyl-benzamide;
2-[[5-[2-[[4-[4-(Dimethylcarbamoyl)phenyl]-1-piperidyl]methyl]-1-methyl-pyrrolo[2,3-b]pyridin-4-yl]-2-pyridyl]amino]acetic acid;
4-[1-[[4-[6-Amino-5-(2H-tetrazol-5-yl)-3-pyridyl]-1-methyl-pyrrolo[2,3-b]pyridin-2-yl]methyl]-4-piperidyl]-N,N-dimethyl-benzamide;
4-[1-[[4-(6-Amino-2,4-dimethyl-3-pyridyl)-1-methyl-pyrrolo[2,3-b]pyridin-2-yl]methyl]-4-piperidyl]-N,N-dimethyl-benzamide;
4-(1-((4-(5-Aminopyridin-2-yl)-1-methyl-1H-pyrrolo[2,3-b]pyridin-2-yl)methyl)piperidin-4-yl)-N,N-dimethylbenzamide;
4-(1-((4-(5-Amino-6-fluoropyridin-2-yl)-1-methyl-1H-pyrrolo[2,3-b]pyridin-2-yl)methyl)piperidin-4-yl)-N,N-dimethylbenzamide;
4-[1-[[4-(6-Aminopyridazin-3-yl)-1-methyl-pyrrolo[2,3-b]pyridin-2-yl]methyl]-4-piperidyl]-N,N-dimethyl-benzamide;
N,N-Dimethyl-4-(1-((1-methyl-4-(5-(methylamino)pyridin-2-yl)-1H-pyrrolo[2,3-b]pyridin-2-yl)methyl)piperidin-4-yl)benzamide;
4-[1-[[1-methyl-4-[5-(methylamino)-2-pyridyl]pyrrolo[2,3-b]pyridin-2-yl]methyl]-4-piperidyl]-N,N-bis(trideuteriomethyl)benzamide;
N,N-Dimethyl-4-[1-[[1-methyl-4-[6-(methylamino)pyridazin-3-yl]pyrrolo[2,3-b]pyridin-2-yl]methyl]-4-piperidyl]benzamide;
4-[1-[[4-(5-Amino-3-fluoro-2-pyridyl)-1-methyl-pyrrolo[2,3-b]pyridin-2-yl]methyl]-4-piperidyl]-N,N-dimethyl-benzamide;
4-[1-[[4-(5-Amino-6-methyl-2-pyridyl)-1-methyl-pyrrolo[2,3-b]pyridin-2-yl]methyl]-4-piperidyl]-N,N-dimethyl-benzamide;
4-[1-[[4-[5-Amino-3-(trifluoromethyl)-2-pyridyl]-1-methyl-pyrrolo[2,3-b]pyridin-2-yl]methyl]-4-piperidyl]-N,N-dimethyl-benzamide;
4-(1-((4-(5-Amino-6-cyanopyridin-2-yl)-1-methyl-1H-pyrrolo[2,3-b]pyridin-2-yl)methyl)piperidin-4-yl)-N,N-dimethylbenzamide
4-[1-[[4-[5-Amino-6-(trifluoromethyl)-2-pyridyl]-1-methyl-pyrrolo[2,3-b]pyridin-2-yl]methyl]-4-piperidyl]-N,N-dimethyl-benzamide
4-(1-((4-(5-Amino-6-chloropyridin-2-yl)-1-methyl-1H-pyrrolo[2,3-b]pyridin-2-yl)methyl)piperidin-4-yl)-N,N-dimethylbenzamide;
Ethyl 3-amino-6-(2-((4-(4-(dimethylcarbamoyl)phenyl)piperidin-1-yl)methyl)-1-methyl-1H-pyrrolo[2,3-b]pyridin-4-yl)picolinate;
3-Amino-6-[2-[[4-[4-(dimethylcarbamoyl)phenyl]-1-piperidyl]methyl]-1-methyl-pyrrolo[2,3-b]pyridin-4-yl]pyridine-2-carboxylic acid;
4-(1-((4-(4-Amino-3-cyanophenyl)-1-methyl-1H-pyrrolo[2,3-b]pyridin-2-yl)methyl)piperidin-4-yl)-N,N-dimethylbenzamide;
4-[(3R)-4-[[4-(6-amino-3-pyridyl)-1-methyl-pyrrolo[2,3-b]pyridin-2-yl]methyl]-3-methyl-piperazin-1-yl]-N,N-dimethyl-benzamide;
4-[(3S)-4-[[4-(6-Amino-3-pyridyl)-1-methyl-pyrrolo[2,3-b]pyridin-2-yl]methyl]-3-methyl-piperazin-1-yl]-N,N-dimethyl-benzamide;
N,N-Dimethyl-4-[4-[[1-methyl-4-[6-(methylamino)-3-pyridyl]pyrrolo[2,3-b]pyridin-2-yl]methyl]piperazin-1-yl]benzamide;
N,N-Dimethyl-6-[4-[[1-methyl-4-[6-(methylamino)-3-pyridyl]pyrrolo[2,3-b]pyridin-2-yl]methyl]piperazin-1-yl]pyridine-3-carboxamide;
N,N-Dimethyl-4-[4-[[1-methyl-4-[4-methyl-6-(methylamino)-3-pyridyl]pyrrolo[2,3-b]pyridin-2-yl]methyl]piperazin-1-yl]benzamide;
N,N-Dimethyl-6-[4-[[1-methyl-4-[5-(methylamino)-2-pyridyl]pyrrolo[2,3-b]pyridin-2-yl]methyl]piperazin-1-yl]pyridine-3-carboxamide;
N,N,3,5-tetramethyl-4-[1-[[1-methyl-4-[5-(methylamino)-2-pyridyl]pyrrolo[2,3-b]pyridin-2-yl]methyl]-3,6-dihydro-2H-pyridin-4-yl]benzamide;
N,N,3-Trimethyl-4-[1-[[1-methyl-4-[5-(methylamino)-2-pyridyl]pyrrolo[2,3-b]pyridin-2-yl]methyl]-3,6-dihydro-2H-pyridin-4-yl]benzamide;
N,N,3-Trimethyl-4-[1-[[1-methyl-4-[5-(methylamino)-2-pyridyl]pyrrolo[2,3-b]pyridin-2-yl]methyl]-4-piperidyl]benzamide;
N,N-Dimethyl-6-[1-[[1-methyl-4-[5-(methylamino)-2-pyridyl]pyrrolo[2,3-b]pyridin-2-yl]methyl]-3,6-dihydro-2H-pyridin-4-yl]pyridine-3-carboxamide;
N,N,5-Trimethyl-6-[1-[[1-methyl-4-[5-(methylamino)-2-pyridyl]pyrrolo[2,3-b]pyridin-2-yl]methyl]-3,6-dihydro-2H-pyridin-4-yl]pyridine-3-carboxamide
N,N,3,5-Tetramethyl-4-[1-[[1-methyl-4-[5-(methylamino)-2-pyridyl]pyrrolo[2,3-b]pyridin-2-yl]methyl]-4-piperidyl]benzamide;
N,N,3-Trimethyl-4-[4-[[1-methyl-4-[5-(methylamino)-2-pyridyl]pyrrolo[2,3-b]pyridin-2-yl]methyl]piperazin-1-yl]benzamide;
N,N,5-trimethyl-6-[1-[[1-methyl-4-[5-(methylamino)-2-pyridyl]pyrrolo[2,3-b]pyridin-2-yl]methyl]-4-piperidyl]pyridine-3-carboxamide;
4-((3S,4S)-3-fluoro-1-((1-methyl-4-(5-(methylamino)pyridin-2-yl)-1H-pyrrolo[2,3-b]pyridin-2-yl)methyl)piperidin-4-yl)-N,N-dimethylbenzamide;
4-((3R,4R)-3-fluoro-1-((1-methyl-4-(5-(methylamino)pyridin-2-yl)-1H-pyrrolo[2,3-b]pyridin-2-yl)methyl)piperidin-4-yl)-N,N-dimethylbenzamide;
4-[4-Fluoro-1-[[1-methyl-4-[5-(methylamino)-2-pyridyl]pyrrolo[2,3-b]pyridin-2-yl]methyl]-4-piperidyl]-N,N-dimethyl-benzamide;
N,N-Dimethyl-4-[(3S,4R)-3-fluoro-1-[[1-methyl-4-[5-(methylamino)-2-pyridyl]pyrrolo[2,3-b]pyridin-2-yl]methyl]-4-piperidyl]benzamide;
N,N-dimethyl-4-[(3R,4S)-3-fluoro-1-[[1-methyl-4-[5-(methylamino)-2-pyridyl]pyrrolo[2,3-b]pyridin-2-yl]methyl]-4-piperidyl]benzamide;
3-Fluoro-N,N-dimethyl-4-[1-[[1-methyl-4-[5-(methylamino)-2-pyridyl]pyrrolo[2,3-b]pyridin-2-yl]methyl]-4-piperidyl]benzamide;
(S)—N,N-dimethyl-4-(1-(1-(1-methyl-4-(5-(methylamino)pyridin-2-yl)-1H-pyrrolo[2,3-b]pyridin-2-yl)ethyl)piperidin-4-yl)benzamide;
(R)—N,N-dimethyl-4-(1-(1-(1-methyl-4-(5-(methylamino)pyridin-2-yl)-1H-pyrrolo[2,3-b]pyridin-2-yl)ethyl)piperidin-4-yl)benzamide;
N,N-dimethyl-4-((2R,4R)-2-methyl-1-((1-methyl-4-(5-(methylamino)pyridin-2-yl)-1H-pyrrolo[2,3-b]pyridin-2-yl)methyl)piperidin-4-yl)benzamide;
N,N-dimethyl-4-((2S,4S)-2-methyl-1-((1-methyl-4-(5-(methylamino)pyridin-2-yl)-1H-pyrrolo[2,3-b]pyridin-2-yl)methyl)piperidin-4-yl)benzamide;
N,N-dimethyl-4-((2S,4R)-2-methyl-1-((1-methyl-4-(5-(methylamino)pyridin-2-yl)-1H-pyrrolo[2,3-b]pyridin-2-yl)methyl)piperidin-4-yl)benzamide;
N,N-dimethyl-4-((2R,4S)-2-methyl-1-((1-methyl-4-(5-(methylamino)pyridin-2-yl)-1H-pyrrolo[2,3-b]pyridin-2-yl)methyl)piperidin-4-yl)benzamide;
N,N-Dimethyl-4-[4-[[1-methyl-4-[6-(methylamino)pyridazin-3-yl]pyrrolo[2,3-b]pyridin-2-yl]methyl]piperazin-1-yl]benzamide;
N,N,3-Trimethyl-4-[1-[[1-methyl-4-[6-(methylamino)pyridazin-3-yl]pyrrolo[2,3-b]pyridin-2-yl]methyl]-4-piperidyl]benzamide;
N,N,3-Trimethyl-4-[4-[[1-methyl-4-[6-(methylamino)pyridazin-3-yl]pyrrolo[2,3-b]pyridin-2-yl]methyl]piperazin-1-yl]benzamide;
3-Methyl-4-[4-[[1-methyl-4-[6-(methylamino)pyridazin-3-yl]pyrrolo[2,3-b]pyridin-2-yl]methyl]piperazin-1-yl]-N,N-bis(trideuteriomethyl)benzamide;
N,N-Dimethyl-6-[1-[[1-methyl-4-[6-(methylamino)pyridazin-3-yl]pyrrolo[2,3-b]pyridin-2-yl]methyl]-4-piperidyl]pyridine-3-carboxamide;
N,N,3-Trimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
3-Methyl-4-[1-[[1-methyl-4-[6-(methylamino)pyridazin-3-yl]pyrrolo[2,3-b]pyridin-2-yl]methyl]-3,6-dihydro-2H-pyridin-4-yl]-N,N-bis(trideuteriomethyl)benzamide;
N,N-Dimethyl-6-[1-[[1-methyl-4-[6-(methylamino)pyridazin-3-yl]pyrrolo[2,3-b]pyridin-2-yl]methyl]-3,6-dihydro-2H-pyridin-4-yl]pyridine-3-carboxamide;
N,N,3,5-Tetramethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
N,N,5-Trimethyl-6-[1-[[1-methyl-4-[6-(methylamino)pyridazin-3-yl]pyrrolo[2,3-b]pyridin-2-yl]methyl]-3,6-dihydro-2H-pyridin-4-yl]pyridine-3-carboxamide
3-Methoxy-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
N,N-Dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]-3-(trifluoromethyl)benzamide;
3-Fluoro-N,N,5-trimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
3-Chloro-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
3-Chloro-5-fluoro-N,N-dimethyl-4-[1-[[1-methyl-4-[6-(methylamino)pyridazin-3-yl]pyrrolo[2,3-b]pyridin-2-yl]methyl]-3,6-dihydro-2H-pyridin-4-yl]benzamide;
N,N-Dimethyl-4-[(2R,4R)-1-[[4-(5-amino-2-pyridyl)-1-methyl-pyrrolo[2,3-b]pyridin-2-yl]methyl]-2-methyl-4-piperidyl]benzamide;
N,N-dimethyl-4-[(2S,4S)-1-[[4-(5-amino-2-pyridyl)-1-methyl-pyrrolo[2,3-b]pyridin-2-yl]methyl]-2-methyl-4-piperidyl]benzamide;
N,N-Dimethyl-4-[(2S,4R)-1-[[4-(6-amino-2-cyano-3-pyridyl)-1-methyl-pyrrolo[2,3-b]pyridin-2-yl]methyl]-2-methyl-4-piperidyl]benzamide;
N,N-dimethyl-4-[(2R,4S)-1-[[4-(6-amino-2-cyano-3-pyridyl)-1-methyl-pyrrolo[2,3-b]pyridin-2-yl]methyl]-2-methyl-4-piperidyl]benzamide;
N,N-Dimethyl-4-[(2R,4R)-1-[[4-(6-amino-2-cyano-3-pyridyl)-1-methyl-pyrrolo[2,3-b]pyridin-2-yl]methyl]-2-methyl-4-piperidyl]benzamide;
N,N-dimethyl-4-[(2S,4S)-1-[[4-(6-amino-2-cyano-3-pyridyl)-1-methyl-pyrrolo[2,3-b]pyridin-2-yl]methyl]-2-methyl-4-piperidyl]benzamide;
N,N-Dimethyl-4-[(2R,4S)-1-[[4-(5-amino-2-pyridyl)-1-methyl-pyrrolo[2,3-b]pyridin-2-yl]methyl]-2-methyl-4-piperidyl]benzamide;
N,N-dimethyl-4-[(2S,4R)-1-[[4-(5-amino-2-pyridyl)-1-methyl-pyrrolo[2,3-b]pyridin-2-yl]methyl]-2-methyl-4-piperidyl]benzamide;
N,N-Dimethyl-4-[(3S,4R)-1-[[4-(4-acetamidophenyl)-1-methyl-pyrrolo[2,3-b]pyridin-2-yl]methyl]-3-fluoro-4-piperidyl]benzamide;
N,N-dimethyl-4-[(3R,4S)-1-[[4-(4-acetamidophenyl)-1-methyl-pyrrolo[2,3-b]pyridin-2-yl]methyl]-3-fluoro-4-piperidyl]benzamide;
4-[1-[[4-[6-(2-Methoxyethylamino)-3-pyridyl]-1-methyl-pyrrolo[2,3-b]pyridin-2-yl]methyl]-2-oxo-4-piperidyl]-N,N-dimethyl-benzamide;
N,N-Dimethyl-4-[1-[[1-methyl-4-[6-(methylamino)-3-pyridyl]pyrrolo[2,3-b]pyridin-2-yl]methyl]-2-oxo-4-piperidyl]benzamide;
N,N-Dimethyl-4-[(4R)-1-[[1-methyl-4-[6-(methylamino)-3-pyridyl]pyrrolo[2,3-b]pyridin-2-yl]methyl]-2-oxo-4-piperidyl]benzamide;
N,N-dimethyl-4-[(4S)-1-[[1-methyl-4-[6-(methylamino)-3-pyridyl]pyrrolo[2,3-b]pyridin-2-yl]methyl]-2-oxo-4-piperidyl]benzamide;
N,N-Dimethyl-4-[4-[[1-methyl-4-[6-(methylamino)-3-pyridyl]pyrrolo[2,3-b]pyridin-2-yl]methyl]-3-oxo-piperazin-1-yl]benzamide;
N,N-Dimethyl-1-[1-[[1-methyl-4-[6-(methylamino)-3-pyridyl]pyrrolo[2,3-b]pyridin-2-yl]methyl]-4-piperidyl]-2-oxo-pyridine-4-carboxamide;
N,N,3-Trimethyl-4-[1-[[1-methyl-4-[6-(methylamino)-3-pyridyl]pyrrolo[2,3-b]pyridin-2-yl]methyl]-3,6-dihydro-2H-pyridin-4-yl]benzamide;
N,N,3-Trimethyl-4-[1-[[1-methyl-4-[6-(methylamino)-3-pyridyl]pyrrolo[2,3-b]pyridin-2-yl]methyl]-4-piperidyl]benzamide;
N,N,3,5-Tetramethyl-4-[1-[[1-methyl-4-[6-(methylamino)-3-pyridyl]pyrrolo[2,3-b]pyridin-2-yl]methyl]-3,6-dihydro-2H-pyridin-4-yl]benzamide;
N,N-Dimethyl-6-[1-[[1-methyl-4-[6-(methylamino)-3-pyridyl]pyrrolo[2,3-b]pyridin-2-yl]methyl]-3,6-dihydro-2H-pyridin-4-yl]pyridine-3-carboxamide;
N,N,5-Trimethyl-6-[1-[[1-methyl-4-[6-(methylamino)-3-pyridyl]pyrrolo[2,3-b]pyridin-2-yl]methyl]-3,6-dihydro-2H-pyridin-4-yl]pyridine-3-carboxamide;
N,N,3,5-Tetramethyl-4-[1-[[1-methyl-4-[6-(methylamino)-3-pyridyl]pyrrolo[2,3-b]pyridin-2-yl]methyl]-4-piperidyl]benzamide;
N,N,3-Trimethyl-4-[4-[[1-methyl-4-[6-(methylamino)-3-pyridyl]pyrrolo[2,3-b]pyridin-2-yl]methyl]piperazin-1-yl]benzamide;
N,N,5-Trimethyl-6-[1-[[1-methyl-4-[6-(methylamino)-3-pyridyl]pyrrolo[2,3-b]pyridin-2-yl]methyl]-4-piperidyl]pyridine-3-carboxamide;
N,N-Dimethyl-4-[(3R,4R)-3-fluoro-1-[[1-methyl-4-[6-(methylamino)-3-pyridyl]pyrrolo[2,3-b]pyridin-2-yl]methyl]-4-piperidyl]benzamide;
N,N-dimethyl-4-[(3S,4S)-3-fluoro-1-[[1-methyl-4-[6-(methylamino)-3-pyridyl]pyrrolo[2,3-b]pyridin-2-yl]methyl]-4-piperidyl]benzamide;
4-[4-Fluoro-1-[[1-methyl-4-[6-(methylamino)-3-pyridyl]pyrrolo[2,3-b]pyridin-2-yl]methyl]-4-piperidyl]-N,N-dimethyl-benzamide;
N,N-Dimethyl-4-[(3R,4S)-3-fluoro-1-[[1-methyl-4-[6-(methylamino)-3-pyridyl]pyrrolo[2,3-b]pyridin-2-yl]methyl]-4-piperidyl]benzamide;
N,N-dimethyl-4-[(3S,4R)-3-fluoro-1-[[1-methyl-4-[6-(methylamino)-3-pyridyl]pyrrolo[2,3-b]pyridin-2-yl]methyl]-4-piperidyl]benzamide;
3-Fluoro-N,N-dimethyl-4-(1-((1-methyl-4-(6-(methylamino)pyridin-3-yl)-1H-pyrrolo[2,3-b]pyridin-2-yl)methyl)piperidin-4-yl)benzamide;
N,N-Dimethyl-4-[1-[[1-methyl-4-[5-(methylamino)-1-oxido-pyridin-1-ium-2-yl]pyrrolo[2,3-b]pyridin-2-yl]methyl]-4-piperidyl]benzamide;
N,N,3-Trimethyl-4-[1-[[1-methyl-4-[5-(methylamino)-1-oxido-pyridin-1-ium-2-yl]pyrrolo[2,3-b]pyridin-2-yl]methyl]-3,6-dihydro-2H-pyridin-4-yl]benzamide
4-[1-[[4-(5-Amino-1-oxido-pyridin-1-ium-2-yl)-1-methyl-pyrrolo[2,3-b]pyridin-2-yl]methyl]-4-piperidyl]-N,N-dimethyl-benzamide;
N,N-Dimethyl-4-[1-[[4-[6-(methylamino)-3-pyridyl]-1-methylsulfonyl-pyrrolo[2,3-b]pyridin-2-yl]methyl]-4-piperidyl]benzamide;
4-[1-[[1-Ethylsulfonyl-4-[6-(methylamino)-3-pyridyl]pyrrolo[2,3-b]pyridin-2-yl]methyl]-4-piperidyl]-N,N-dimethyl-benzamide;
4-[1-[[1-(2,2-Dimethylpropyl)-4-[6-(methylamino)-3-pyridyl]pyrrolo[2,3-b]pyridin-2-yl]methyl]-4-piperidyl]-N,N-dimethyl-benzamide and
4-(1-((4-(5-Aminopyridin-2-yl)-3-fluoro-1-methyl-1H-pyrrolo[2,3-b]pyridin-2-yl)methyl)piperidin-4-yl)-N,N-dimethylbenzamide or a pharmaceutically acceptable salt or stereoisomer thereof:

Embodiment 47. A compound according to any one of embodiments 1-46 for use in therapy.

Embodiment 48. A compound according to embodiment 47 for use in the treatment of a disease, disorder or condition, which disease, disorder or condition is responsive of modulation of STAT-6.

Embodiment 49. A compound according to embodiment 47 for use in the treatment of autoimmune diseases.

Embodiment 50. A compound according to embodiment 47 for use in the treatment diseases characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast cancer and solid fibrous cancers either as a stand-alone treatment or in combination with other anticancer drugs such as check point inhibitors.

Embodiment 51. A pharmaceutical composition comprising a compound according to any one of embodiments 1-46 together with a pharmaceutically acceptable vehicle or excipient or pharmaceutically acceptable carrier(s).

1. A compound according to formula (I): 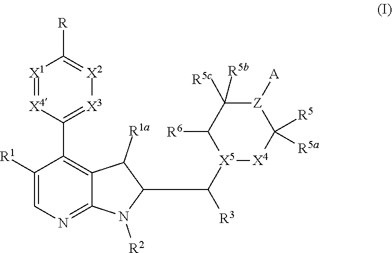
A is selected from 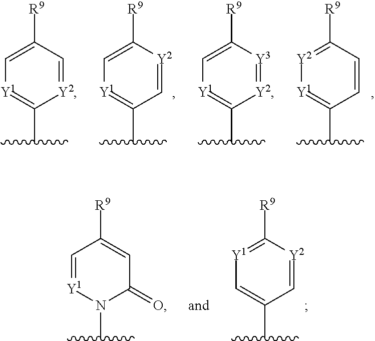
X¹ is selected from CR¹¹ and N; X² is selected from CR¹² and N; X³ is selected from CR¹³ and N, and X⁴′ is selected from CR¹⁴ and N, and wherein when X⁴′ is N the N may be an N-oxide, or X¹ is CR¹¹ and taken together with R forms a 5-membered heteroaryl ring which is optionally substituted with C(O)CF₃;
X⁴ is selected from CR⁴R⁴ and CO;
X⁵ is selected from N and oxidized N;
Y¹ is selected from N and CR⁷;
Y² is selected from N and CR⁸;
Y³ is selected from N and CR¹⁷;
Z is selected from N and CR¹⁰;
R is selected from NHR⁰ and —CONHR⁰;
R⁰ is selected from hydrogen, C₁₋₄alkyl, C₃₋₄ cycloalkyl, C₃₋₄ cycloalkyl-C₁₋₄alkyl, phenyl-C₁₋₄alkyl, halo-C₁₋₄alkyl, —(CH₂)ₙ—O—R′, —(CH₂)ₙ—CO—C₁₋₄alkyl and —(CH₂)ₙ—CO₂R′, wherein said phenyl is optionally substituted with one or more substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein R′ and R″ are each independently selected from hydrogen and C₁₋₄alkyl and n is 0, 1 or 2;
R¹ and R^{1a} are independently selected from hydrogen, fluoro and C₁₋₄alkyl;
R² is selected from hydrogen, C₁₋₅alkyl, —SO₂—C₁₋₄alkyl and deuterated C₁₋₄alkyl, wherein said C₁₋₅alkyl may optionally be substituted with one or more fluoro;
R³ is independently selected from hydrogen and C₁₋₄alkyl;
each R⁴ and R⁶ are independently selected from hydrogen and C₁₋₄alkyl and C₁₋₄alkoxy, wherein said C₁₋₄alkyl and C₁₋₄alkoxy may optionally be substituted with one or more halogen;
R⁵ is selected from hydrogen, halogen, C₁₋₄alkyl and C₁₋₄alkoxy, wherein said C₁₋₄alkyl and C₁₋₄alkoxy may optionally be substituted with one or more halogen;
R¹⁰ is selected from hydrogen and fluoro; or
R⁵ and R¹⁰ together form a bond between the two carbons to which they are attached;
R^{5a} is hydrogen;
or R⁵ and R^{5a} are both fluoro or R⁵ and R^{5a} together with the atom attached thereto form a CO group;
R^{5b} and R^{5c} are each independently selected from hydrogen and fluoro;
R⁷, R⁸, and R¹⁷ are each independently selected from hydrogen, halogen, cyano, C₁₋₄alkyl and C₁₋₄alkoxy, and C₃₋₄cycloalkyl, wherein said C₁₋₄alkyl and C₁₋₄alkoxy may optionally be substituted with one or more halogen;
R⁹ is —CONR¹⁵R¹⁶;
R¹¹, R¹², R¹³ and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted with one or more substituents independently selected from halogen, hydroxy and C₁₋₄alkoxy, wherein m is 1, 2 or 3, and wherein R′ and R″ are each independently selected from hydrogen and C₁₋₄alkyl; or
R¹¹ and R⁰ together with the atoms attached thereto form a 5-6 membered heteroaromatic or heterocyclic ring containing one or two N atoms, wherein said 5-6 membered heteroaromatic ring or heterocyclic ring containing one or two N atoms is optionally substituted with one or more substituents independently selected from halogen, C₁₋₄alkyl, halo-C₁₋₄alkyl, C₃₋₄ cycloalkyl, hydroxy, C₁₋₄alkoxy, cyano, —C(O)CF₃, —NR′R″, —CONR′R″ and —CO₂R′, wherein said C₁₋₄alkyl may optionally be substituted with one or more substituents independently selected from halogen and C₁₋₄alkoxy, and wherein R′ and R″ are each independently selected from hydrogen and C₁₋₄alkyl;
R¹⁵ and R¹⁶ are independently selected from hydrogen, C₁₋₄alkyl, and deuterated C₁₋₄alkyl;
or a pharmaceutically acceptable salt or stereoisomer thereof.

2. The compound according to claim 1 having the formula (I′): 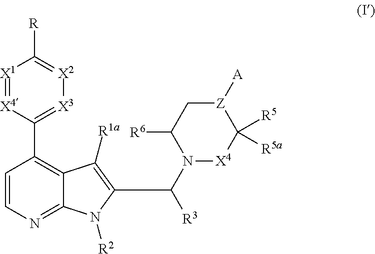
wherein A is selected from 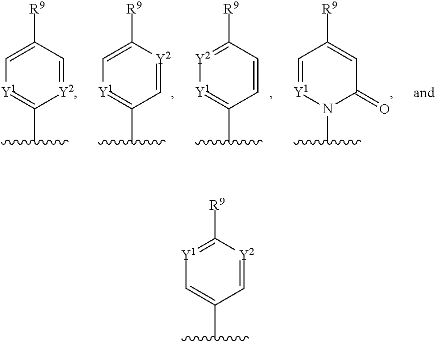
R³ is C₁₋₄alkyl;
R¹¹, R¹², R¹³ and R¹⁴ are each independently selected from hydrogen, halogen, C₁₋₄alkyl, C₃₋₄ cycloalkyl, C₁₋₄alkoxy, hydroxy, cyano, —NR′R″, —CONR′R″, —CO₂R′, —CO—CO₂R′, —CO—(CH₂)ₘOH, tetrazolyl and —SO₂—C₁₋₄alkyl, wherein said C₁₋₄alkyl and C₁₋₄alkoxy are optionally substituted with one or more substituents independently selected from halogen, hydroxy and C₁₋₄alkoxy, wherein m is 1, 2 or 3, and wherein R′ and R″ are each independently selected from hydrogen and C₁₋₄alkyl; or
R¹¹ and R⁰ together with the atoms attached thereto form a 5-6 membered heteroaromatic containing one or two N atoms, wherein said 5-6 membered heteroaromatic is optionally substituted with one or more substituents independently selected from halogen, C₁₋₄alkyl, halo-C₁₋₄alkyl, C₃₋₄ cycloalkyl, hydroxy, C₁₋₄alkoxy, cyano, —NR′R″, —CONR′R″ and —CO₂R′, wherein said C₁₋₄alkyl may optionally be substituted with one or more substituents independently selected from halogen and C₁₋₄alkoxy, and wherein R′ and R″ are each independently selected from hydrogen and C₁₋₄alkyl;
or a pharmaceutically acceptable salt or stereoisomer thereof.

3. The compound according to claim 1, wherein X¹ is selected from CR¹¹ and N; X² is selected from CR¹² and N; X³ is selected from CR¹³ and N and X⁴′ is selected from CR⁴ and N.

4. The compound according to claim 1, wherein: X¹ is N, X² is CR¹², X³ is CR¹³ and X⁴′ is CR¹⁴.

5. The compound according to claim 1, wherein:
X¹ is CR¹¹, X² is CR¹², X⁴′ is CR¹⁴ and X³ is N;
X¹ is CR¹¹, X² is CR¹², X³ is CR¹³ and X⁴′ is CR¹⁴:
X¹ is N; X² is selected from CR¹², X³ is N and X⁴′ is CR¹⁴:
X¹ is N, X² is N, X³ is CR¹³ and X⁴′ is CR¹⁴:
X¹, X², X³ is N and X⁴′ is CR¹⁴:
X¹ is CR¹¹, X² is CR¹², and X³ and X⁴′ is N;
X¹ is N, X² is CR¹², and X³ and X⁴′ is N; or
X¹ is CR¹¹, X² is N, X³ is N and X⁴′ is CR¹⁴.

6.-13. (canceled)

14. The compound according to claim 1, wherein Z is CR¹⁰ and R⁵ and R¹⁰ together form a bond.

15. The compound according to claim 1, wherein Z is CR¹⁰ and R¹⁰ is hydrogen.

16. The compound according to claim 1, wherein Y¹ is CR⁷ and Y² is CR⁸.

17. The compound according to claim 1, wherein Y¹ is CR⁷ and Y² is N; or Y¹ and Y² is N.

18. (canceled)

19. The compound according to claim 16, wherein
(a) both of R⁷ and R⁸ are C₁₋₄alkyl;
(b) both of R⁷ and R⁸ are halogen;
(c) one of R⁷ and R⁸ is C₁₋₄alkyl and the other is halogen;
(d) one of R⁷ and R⁸ is C₁₋₄alkyl and the other is hydrogen;
(e) one of R⁷ and R⁸ is halogen and the other is hydrogen;
(f) one of R⁷ and R⁸ is cyclopropyl; or
(g) one R⁷ and R⁸ is cyano.

20. A compound according to claim 16, wherein both of R⁷ and R⁸ are hydrogen.

21. A compound according to claim 19, wherein one of R⁷ and R⁸ is hydrogen and the other is fluoro, cyano, or cyclopropyl.

22. The compound according to claim 1, wherein R is NHR⁰ and R⁰ is selected from C₁₋₄alkyl, —(CH₂)ₙ—O—C₁₋₄alkyl, —(CH₂)ₙ—CO—C₁₋₄alkyl and —(CH₂)ₙ—CO₂R′ where n is 0, 1 or 2.

23. A compound according to claim 1, wherein R⁹ is selected from CON(CH₃)₂ and —CON(CD₃)₂.

24. A compound according to claim 1, wherein R¹ is hydrogen, R² is hydrogen and R³ is selected from hydrogen and methyl.

25. A compound selected from:

| Example | Structure |
|---|---|
| 1aa2 1aa2′ | 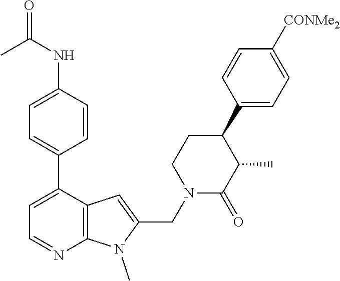 |
| | Single unknown isomer |
| | 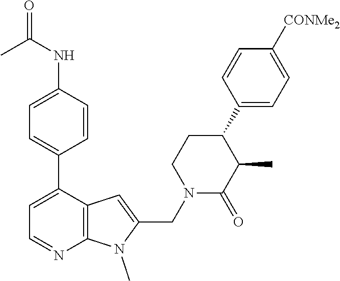 |
| 1ad1 | 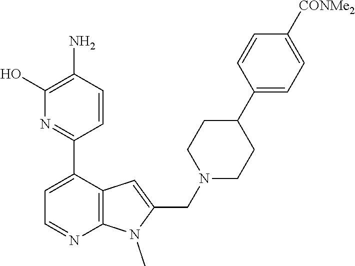 |
| 1ad2 | 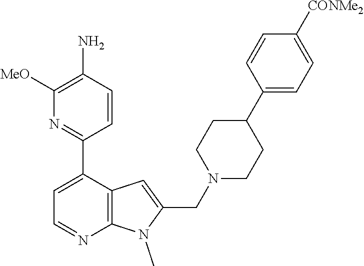 |
| 1ad3 | 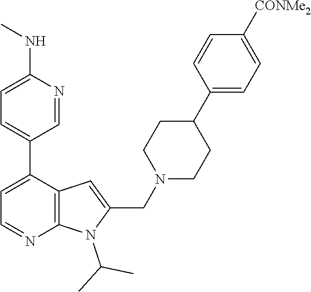 |
| 1ae4 1ae5 | 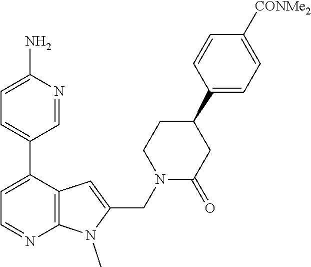 |
| | 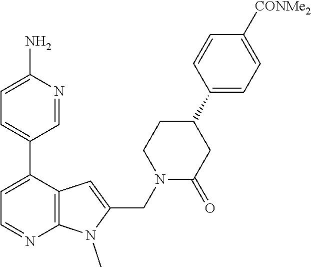 |
| 1ae7 | 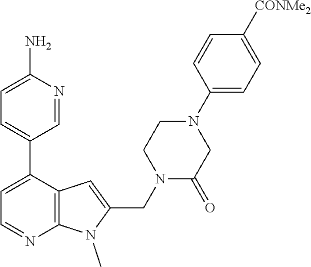 |
| 1ae10 | 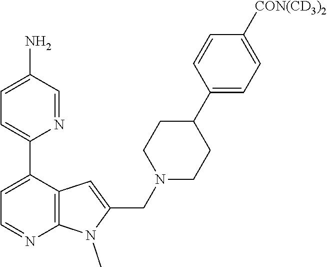 |
| 1ae14 | 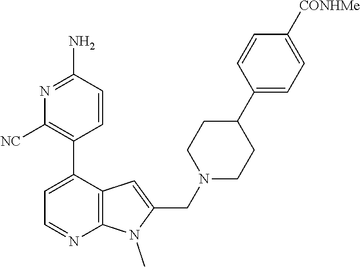 |
| 1ae15 | 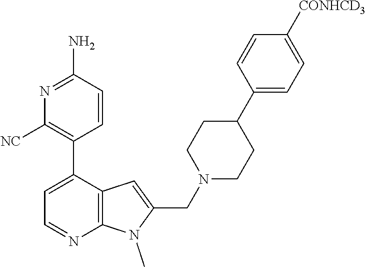 |
| 1ae19 | 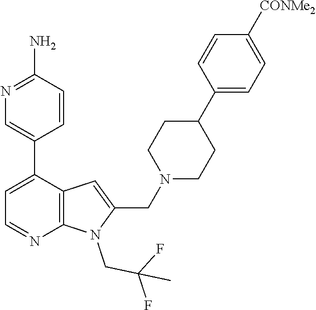 |
| 1ae22 | 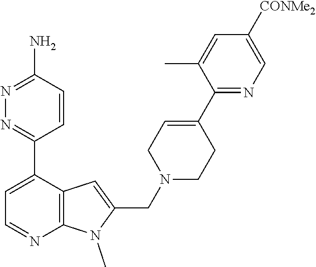 |
| 1af7 1af8 | 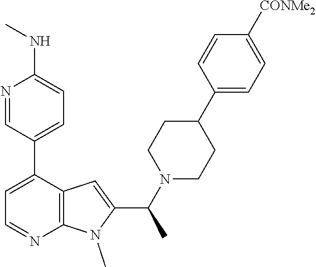 |
| | 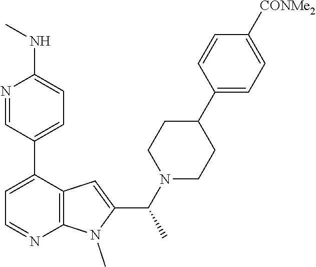 |
| 1af11 1af12 | 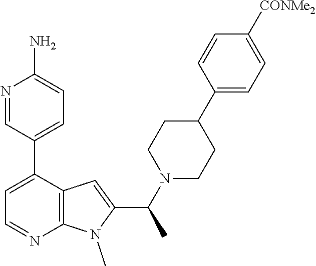 |
| | 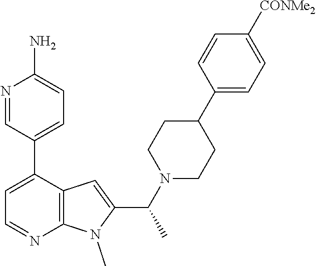 |
| 1af13 1af14 | 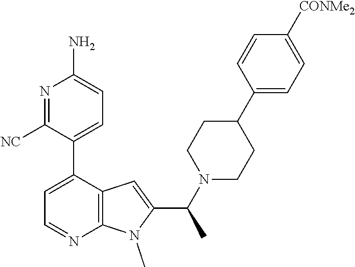 |
| | 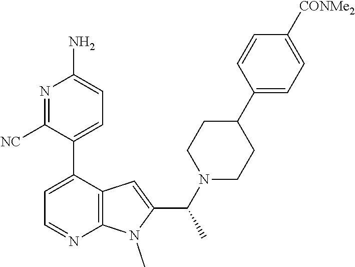 |
| 1af15 1af16 | 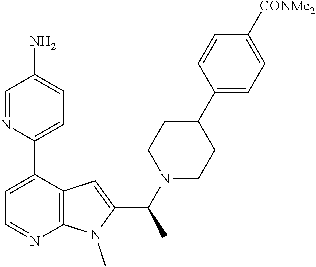 |
| | 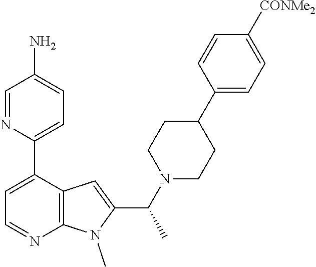 |
| 1af17 1af18 | 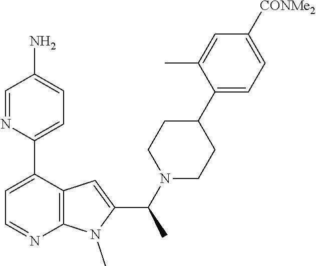 |
| | 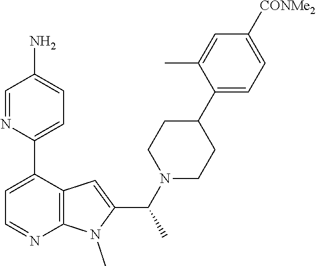 |
| 1af19 1af20 | 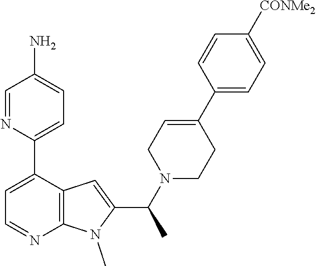 |
| | 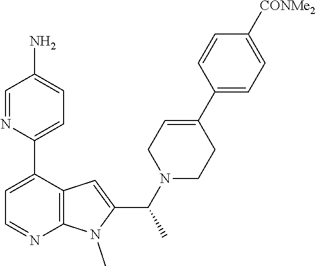 |
| 1af21 1af23 | 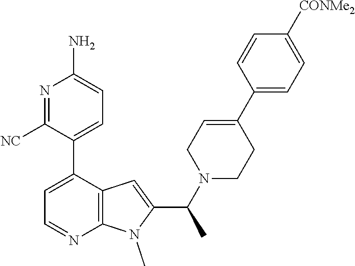 |
| | 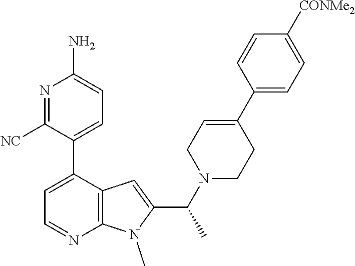 |
| 1af22 1af24 | 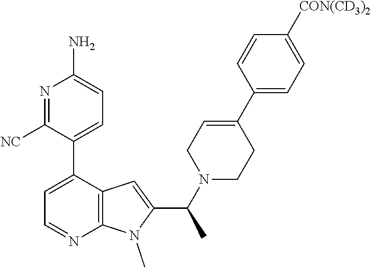 |
| | 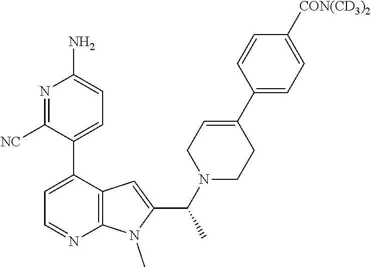 |
| 1af25 1af26 | 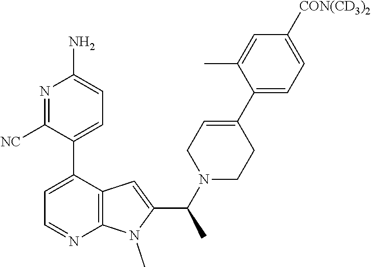 |
| | 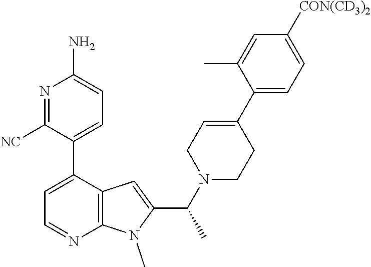 |
| 1af27 1af28 | 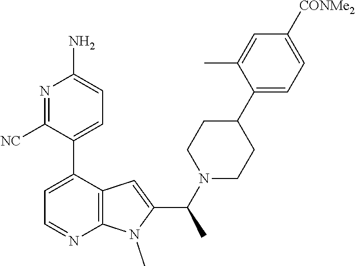 |
| | 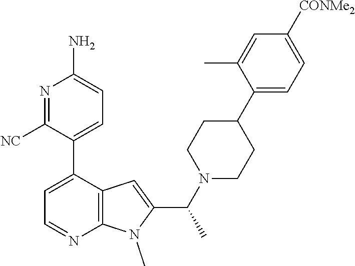 |
| 1af29 1af30 | 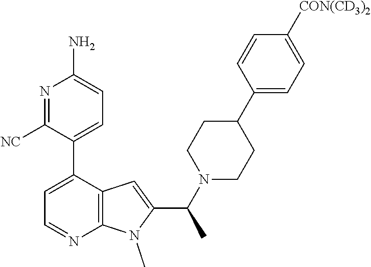 |
| | 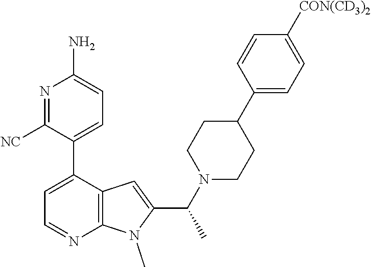 |
| 1af31 1af33 | 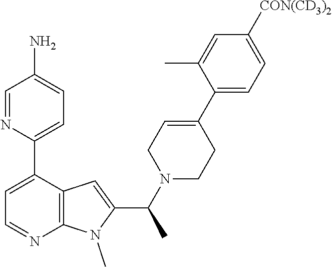 |
| | 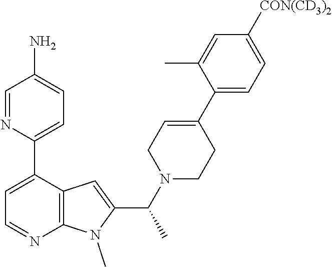 |
| 1af32 1af34 | 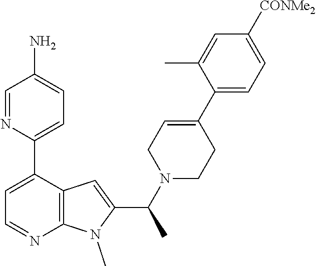 |
| | 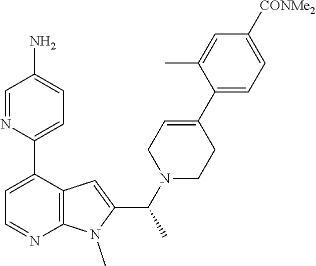 |
| 1af35 1af36 | 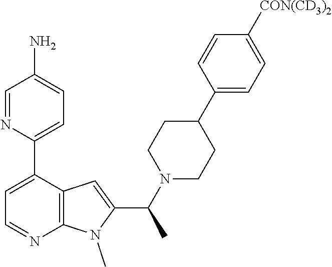 |
| | 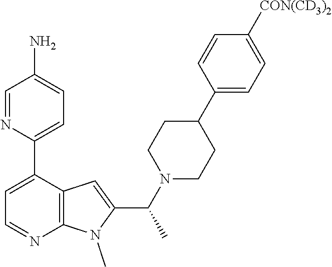 |
| 1af37 1af38 | 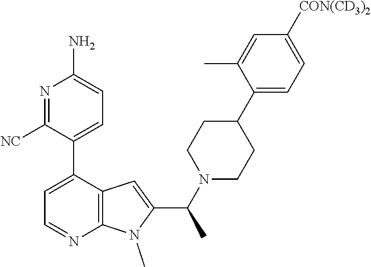 |
| | 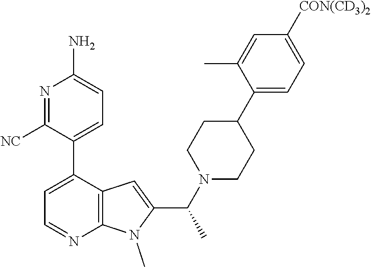 |
| 1af39 1af40 | 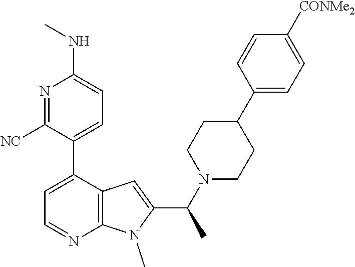 |
| | 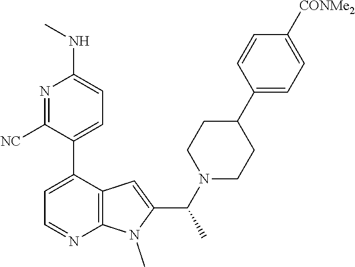 |
| 1af41 1af42 | 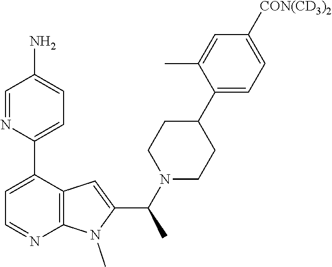 |
| | 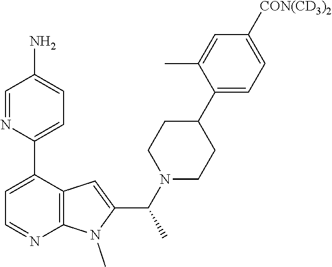 |
| 1af43 1af46 | 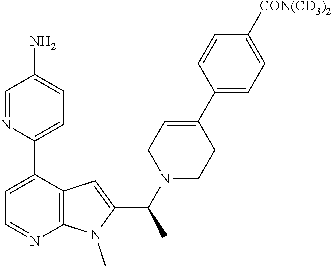 |
| | 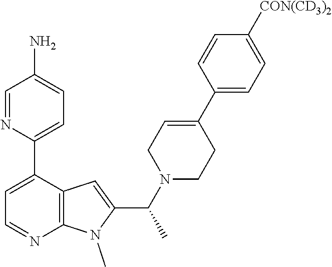 |
| 1af44 1af45 | 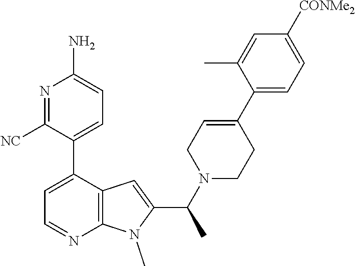 |
| | 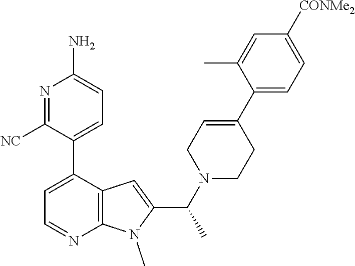 |
| 1af50 | 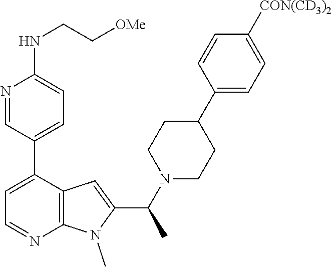 |
| 1af51 | 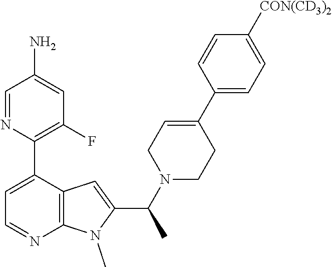 |
| 1af52 | 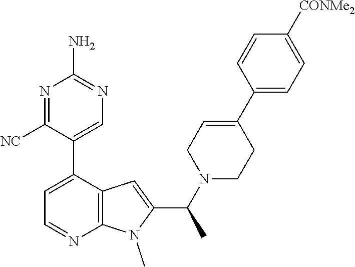 |
| 1af53 | 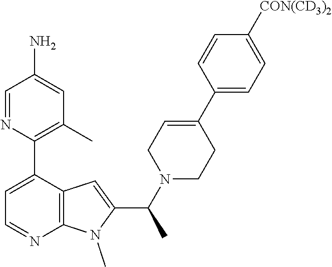 |
| 1af54 | 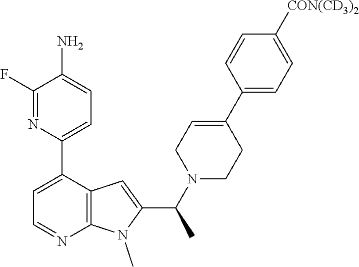 |
| 1af55 | 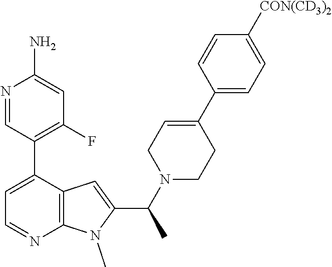 |
| 1af56 | 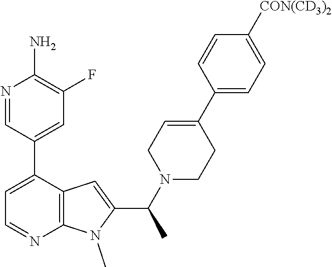 |
| 1af57 | 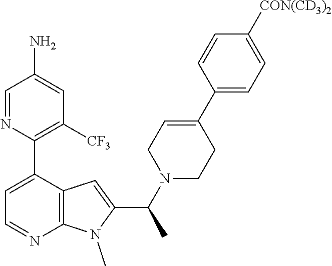 |
| 1af58 | 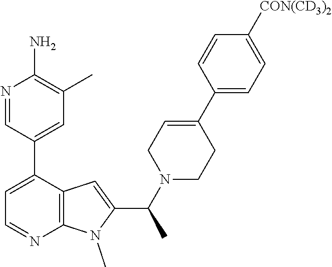 |
| 1af59 | 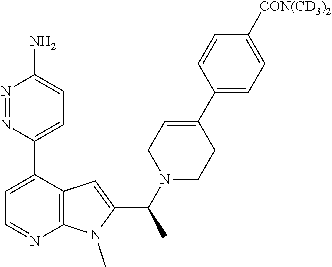 |
| 1af60 | 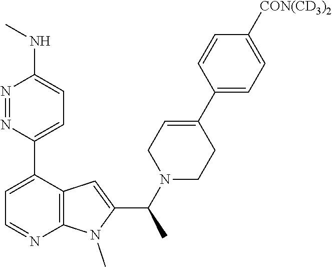 |
| 1af64 | 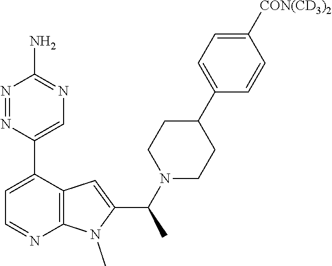 |
| 1af66 | 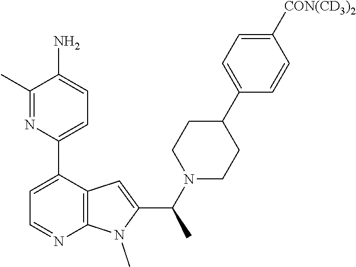 |
| 1af67 | 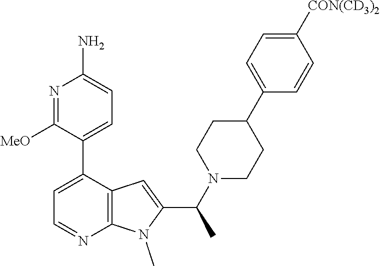 |
| 1af68 | 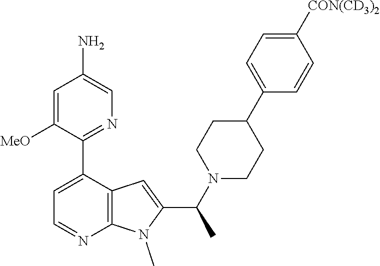 |
| 1af69 | 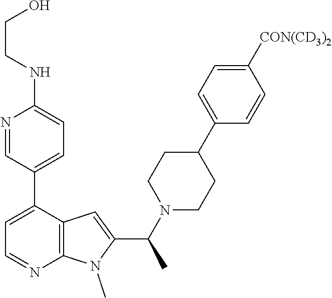 |
| 1af70 | 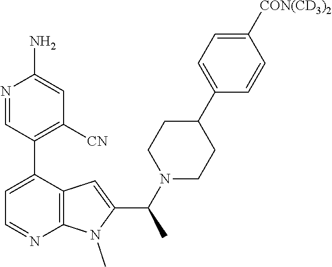 |
| 1af72 | 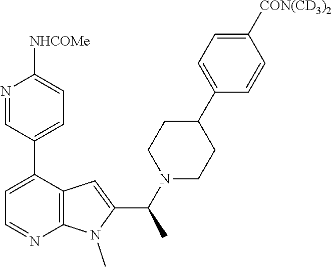 |
| 1af73 | 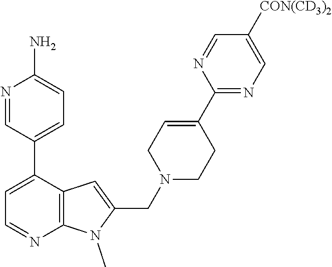 |
| 1af75 | 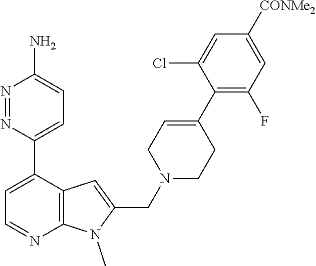 |
| 1af76 | 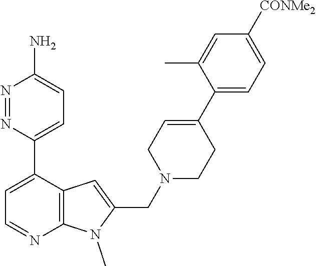 |
| 1af77 | 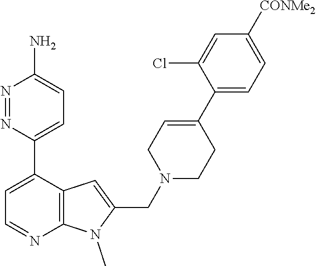 |
| 1af78 | 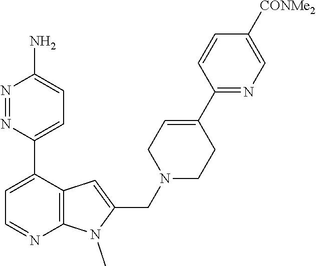 |
| 1af79 | 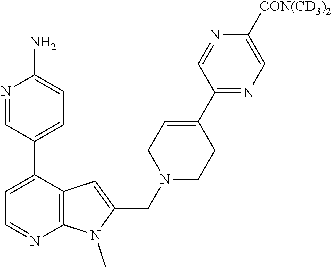 |
| 1af80 | 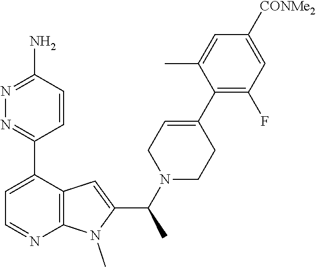 |
| 1af81 | 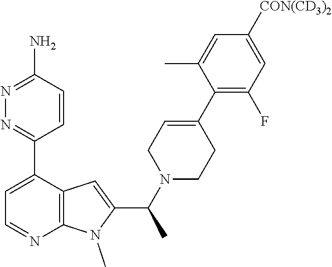 |
| 1j6 | 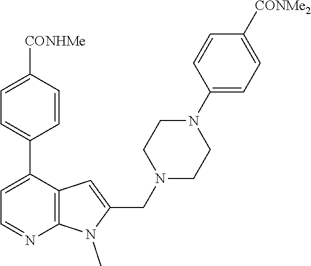 |
| 1j7 | 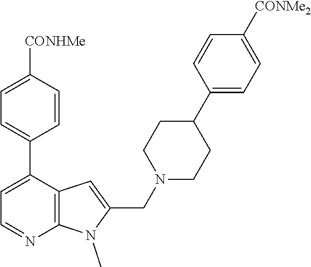 |
| 1j13 | 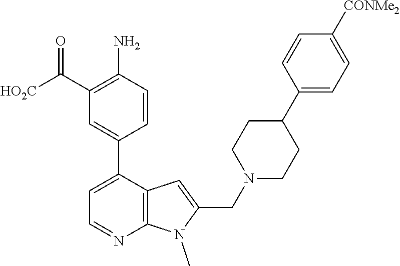 |
| 1k2 | 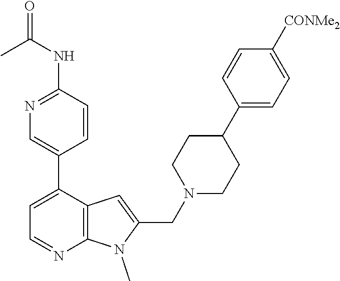 |
| 1k3 | 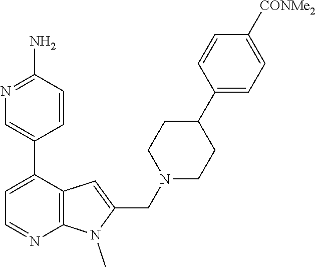 |
| 1k6 | 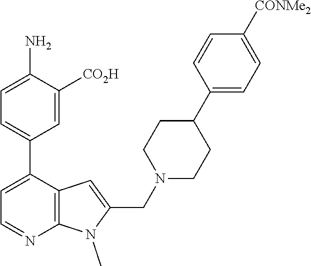 |
| 1k7 | 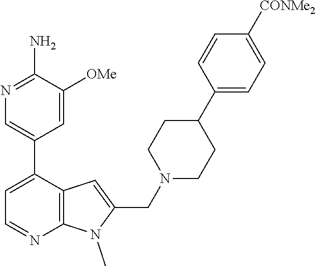 |
| 1k8 | 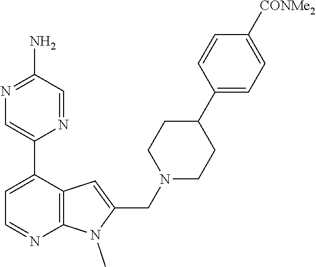 |
| 1k9 | 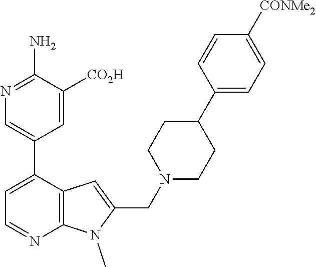 |
| 1k10 | 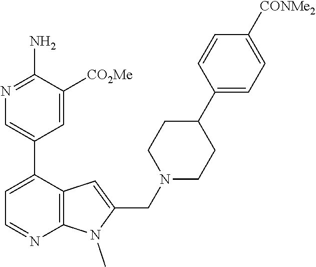 |
| 1k11 | 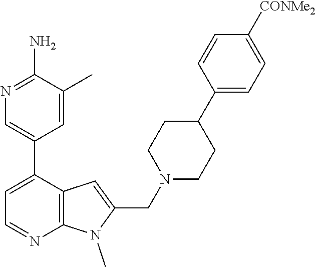 |
| 1k12 | 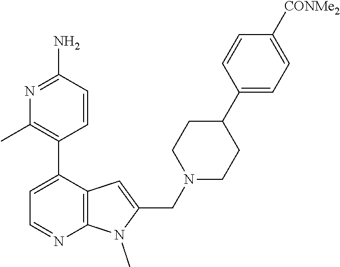 |
| 1k13 | 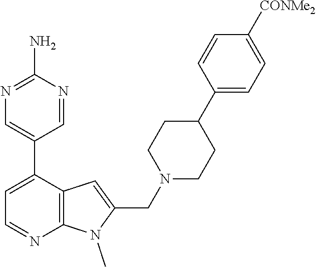 |
| 1k14 | 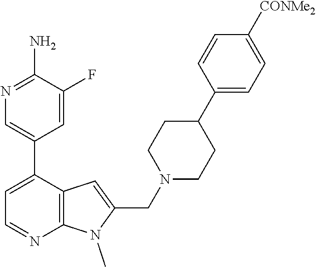 |
| 1k15 | 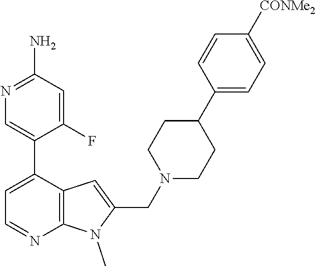 |
| 1k16 | 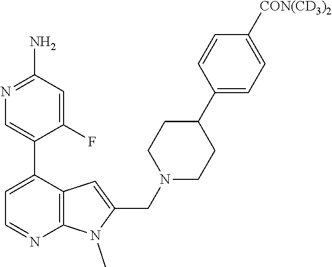 |
| 1k17 | 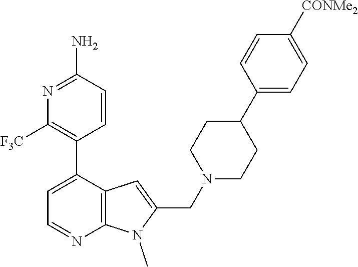 |
| 1k18 | 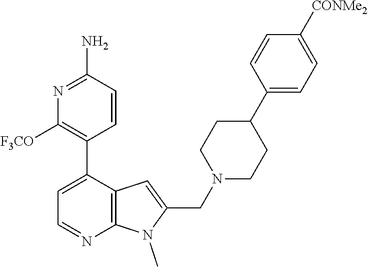 |
| 1k19 | 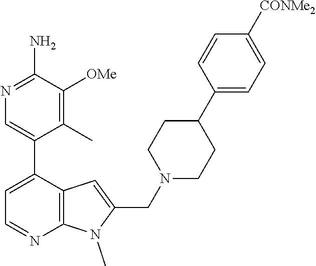 |
| 1k20 | 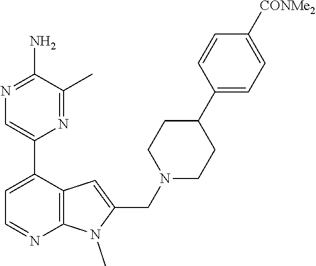 |
| 1k21 | 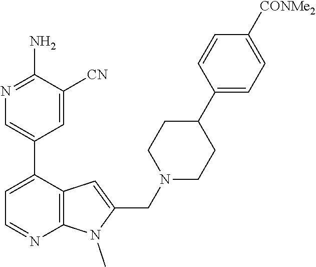 |
| 1k22 | 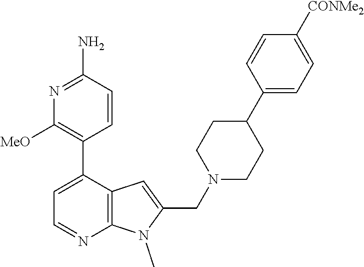 |
| 1k23 | 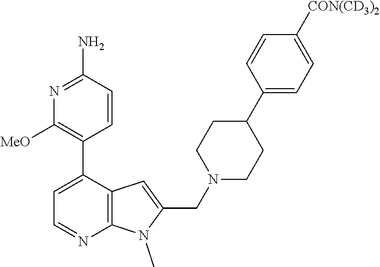 |
| 1k24 | 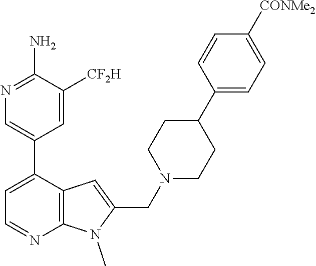 |
| 1k25 | 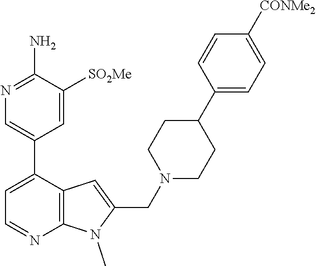 |
| 1k26 | 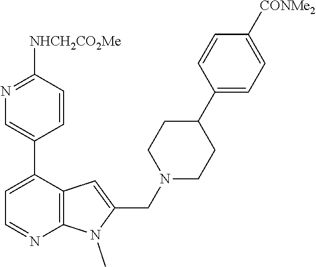 |
| 1k27 | 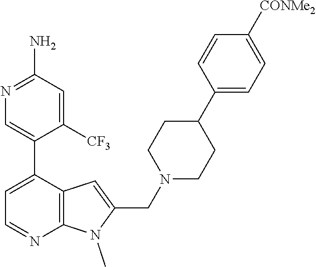 |
| 1k28 | 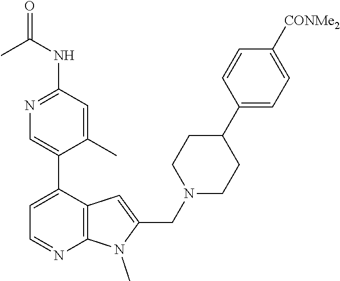 |
| 1k30 | 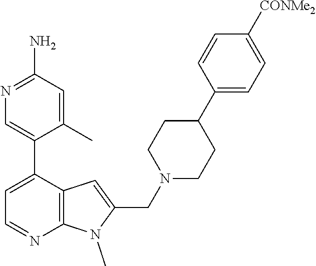 |
| 1k31 | 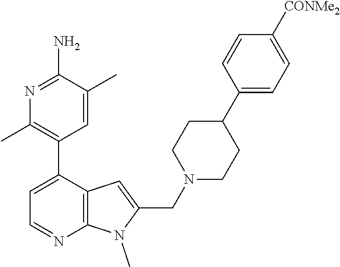 |
| 1k32 | 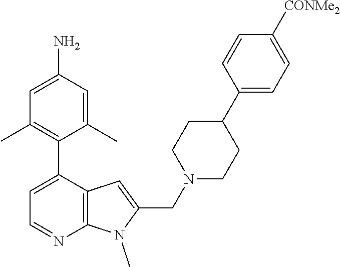 |
| 1k33 | 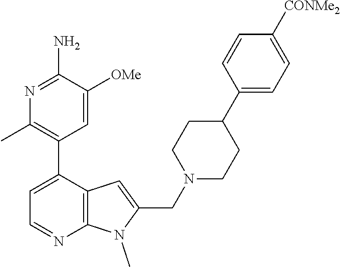 |
| 1k34 | 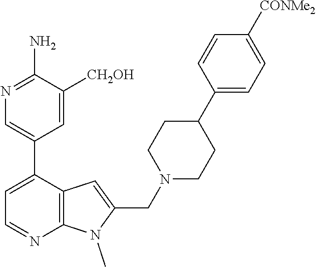 |
| 1k35 | 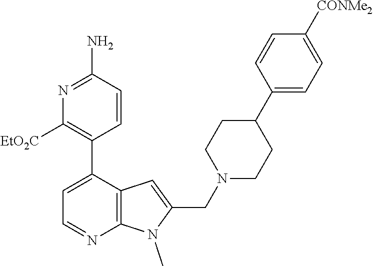 |
| 1k36 | 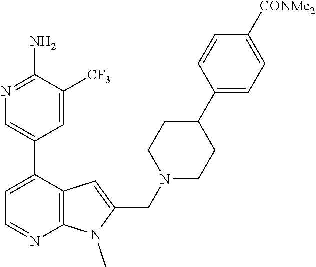 |
| 1k37 | 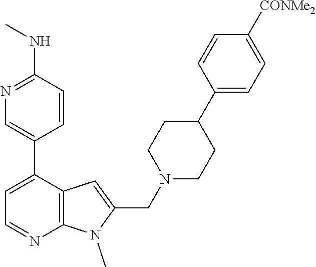 |
| 1k38 | 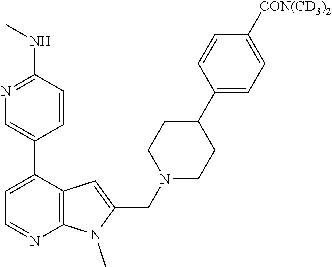 |
| 1k39 | 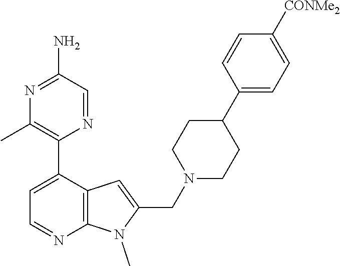 |
| 1k40 | 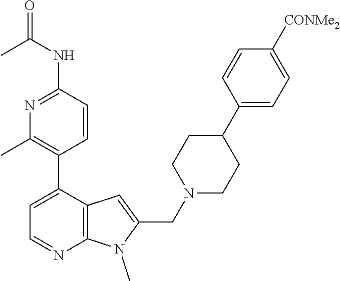 |
| 1k41 | 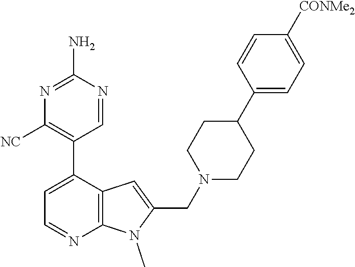 |
| 1k42 | 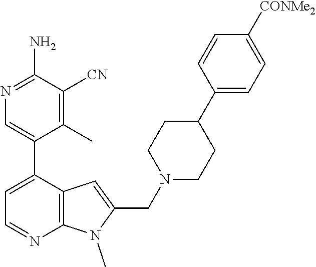 |
| 1k43 | 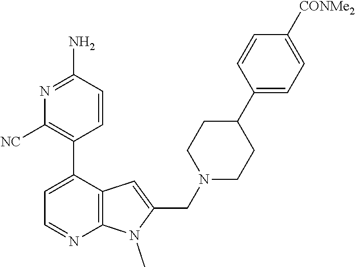 |
| 1k44 | 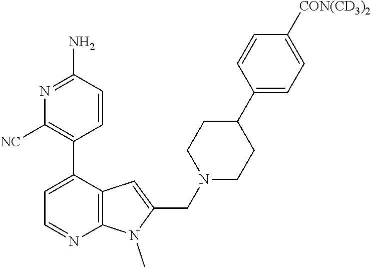 |
| 1k45 | 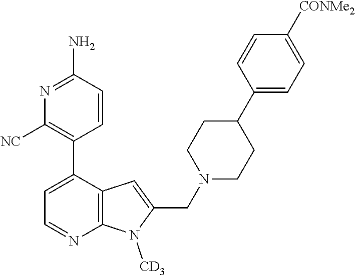 |
| 1k47 | 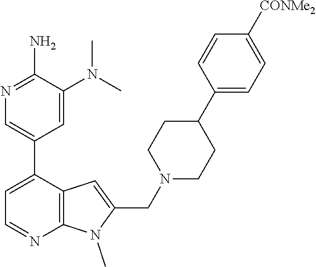 |
| 1k48 | 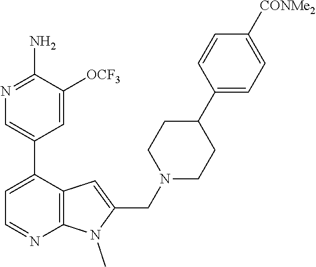 |
| 1k49 | 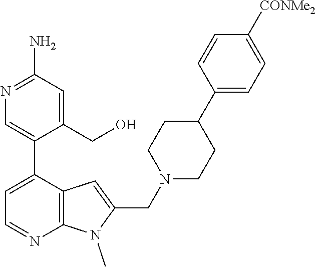 |
| 1k50 | 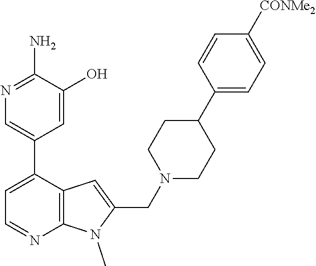 |
| 1k51 | 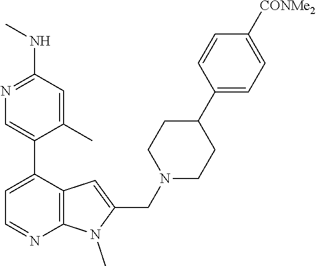 |
| 1k52 | 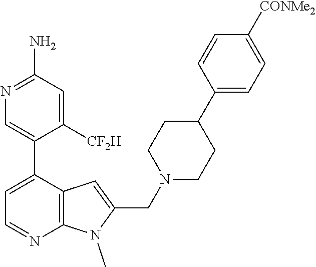 |
| 1k53 | 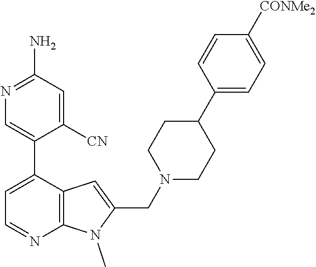 |
| 1k54 | 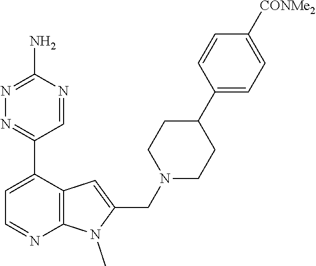 |
| 1k55 | 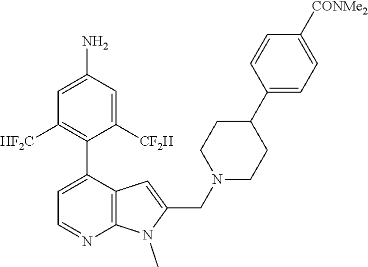 |
| 1k56 | 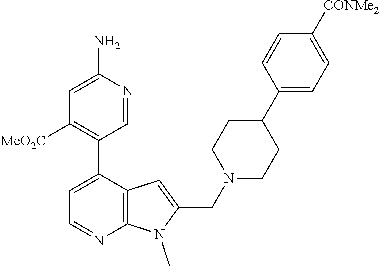 |
| 1k57 | 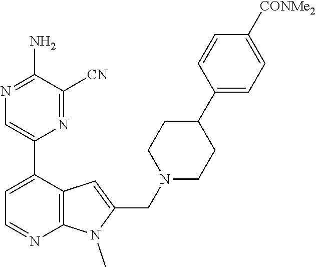 |
| 1k58 | 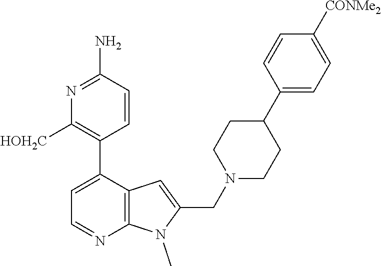 |
| 1k59 | 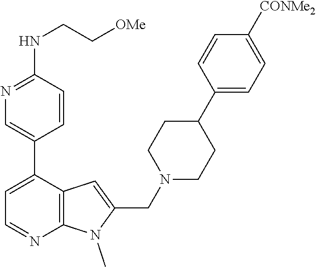 |
| 1k60 | 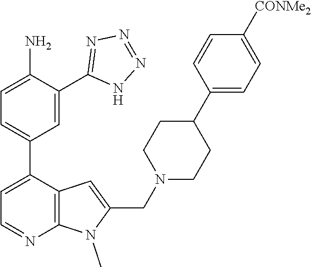 |
| 1k61 | 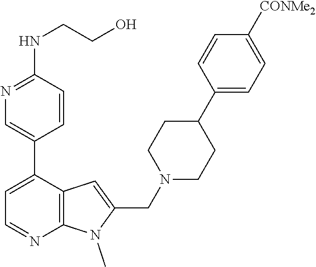 |
| 1k63 | 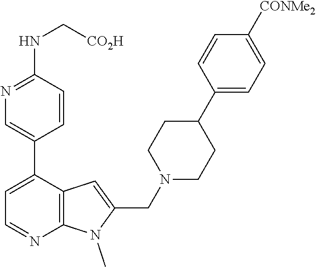 |
| 1k64 | 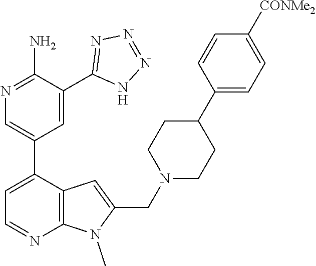 |
| 1k65 | 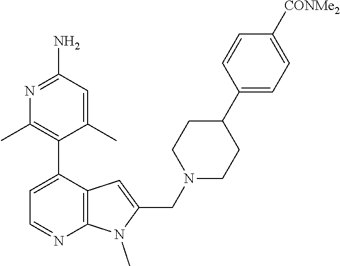 |
| 1k66 | 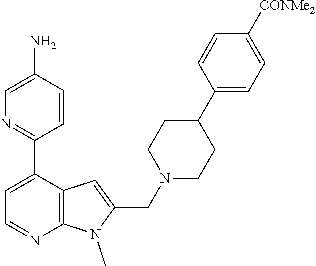 |
| 1k67 | 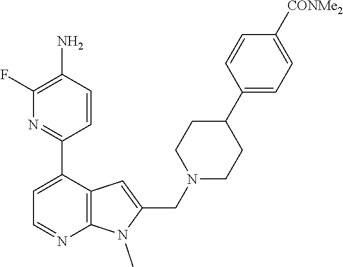 |
| 1k68 | 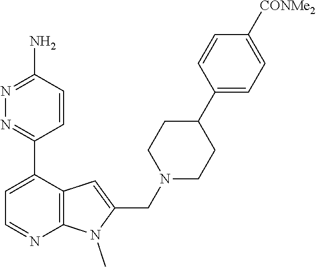 |
| 1k69 | 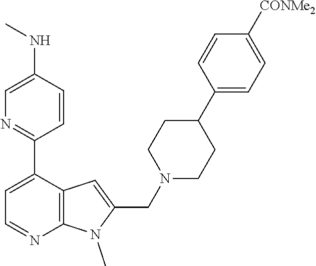 |
| 1k70 | 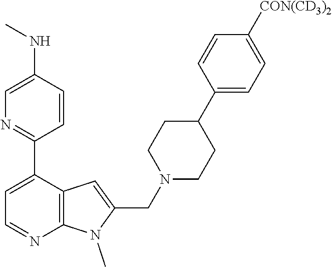 |
| 1k71 | 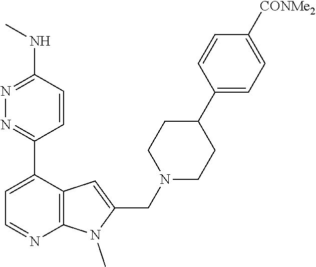 |
| 1k72 | 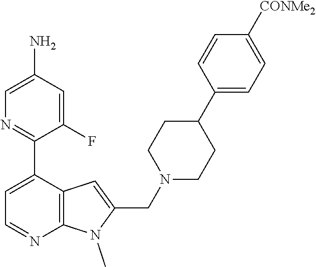 |
| 1k73 | 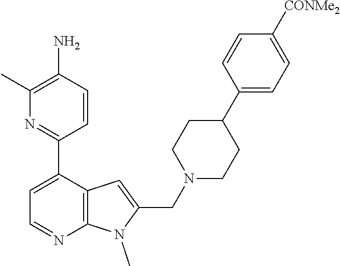 |
| 1k74 | 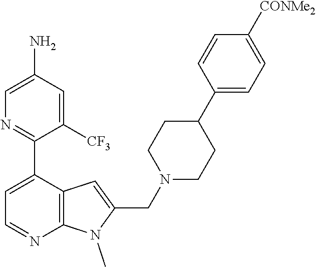 |
| 1k75 | 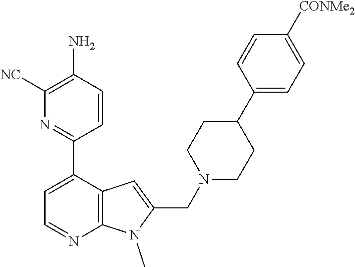 |
| 1k76 | 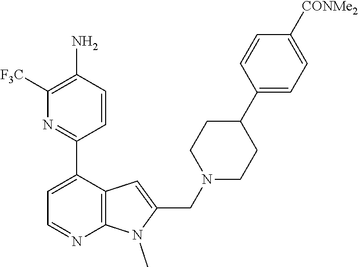 |
| 1k77 | 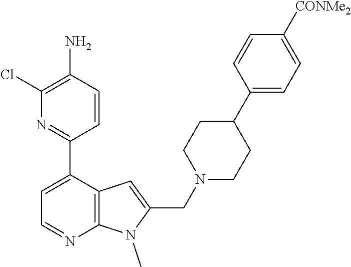 |
| 1k78 | 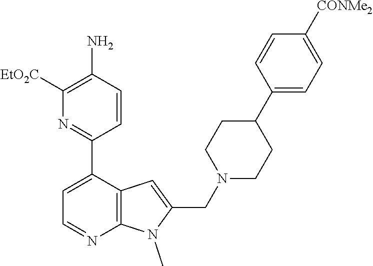 |
| 1k79 | 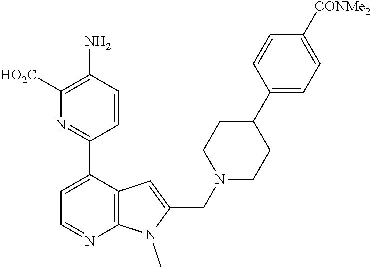 |
| 1k80 | 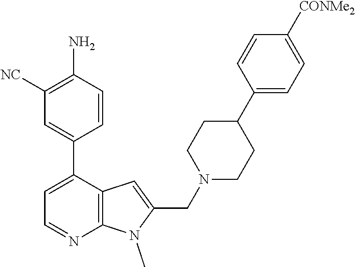 |
| 1m145 | 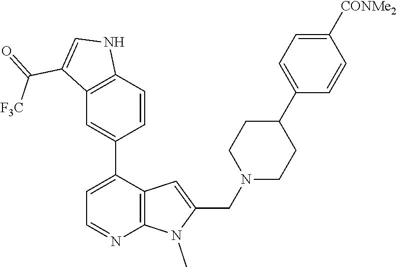 |
| 1n4 | 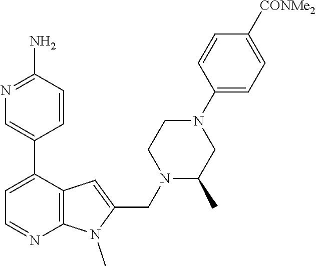 |
| 1n5 | 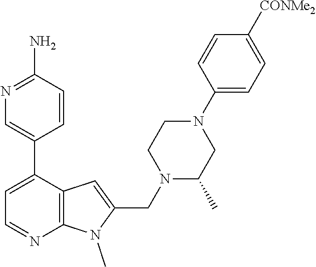 |
| 1n6 | 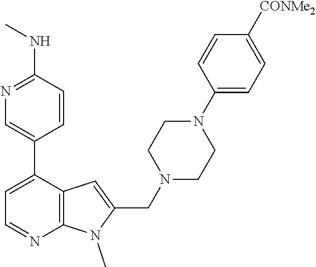 |
| 1n7 | 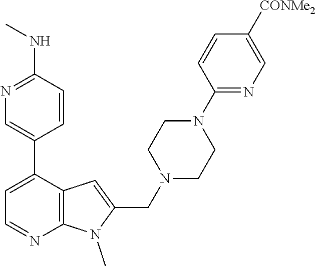 |
| 1n8 | 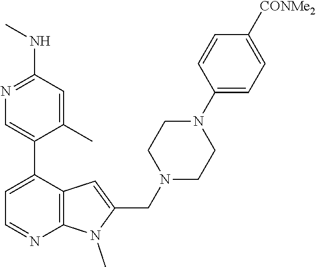 |
| 1n9 | 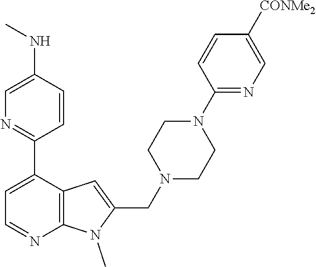 |
| 1o1 | 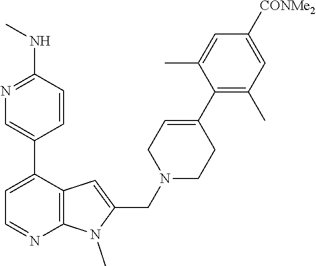 |
| 1o2 | 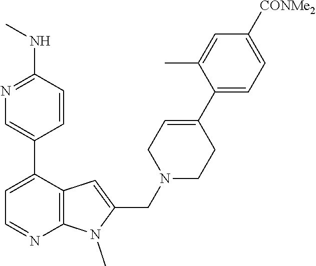 |
| 1o3 | 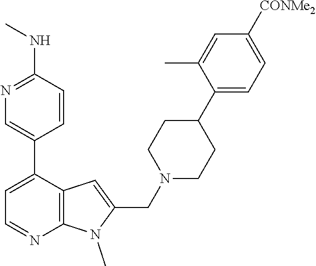 |
| 1o4 | 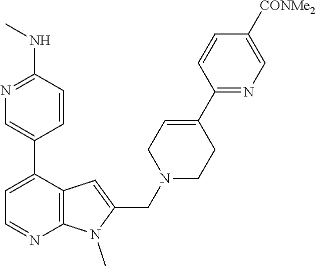 |
| 1o5 | 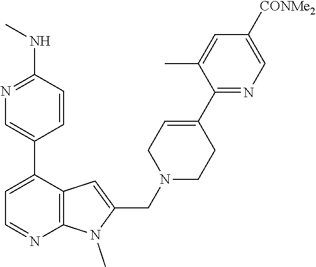 |
| 1o6 | 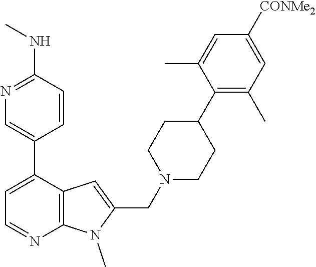 |
| 1o7 | 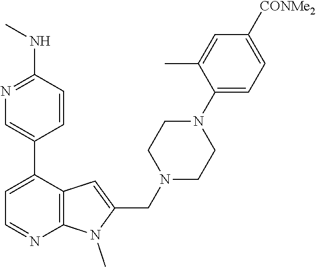 |
| 1o8 | 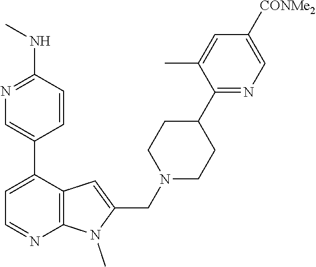 |
| 1o17 1o18 | 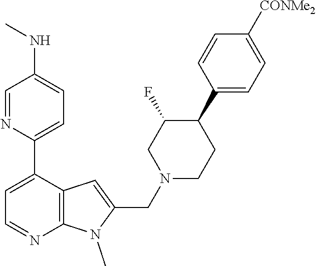 |
| | 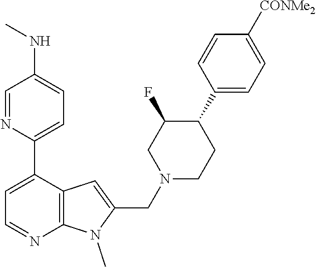 |
| 1o19 | 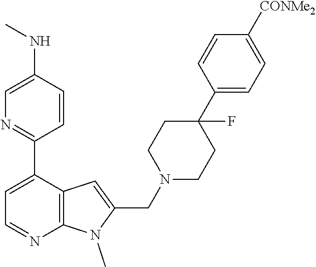 |
| 1o20 1o21 | 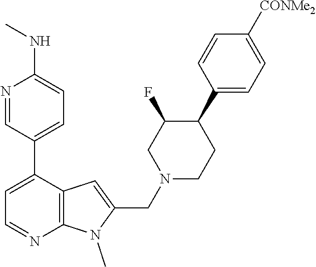 |
| | 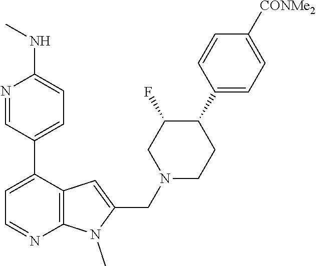 |
| 1o23 | 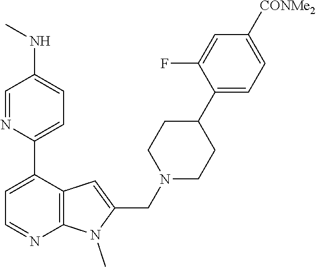 |
| 1o24 1o25 | 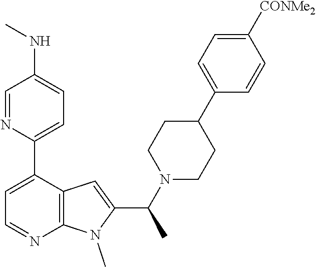 |
| | 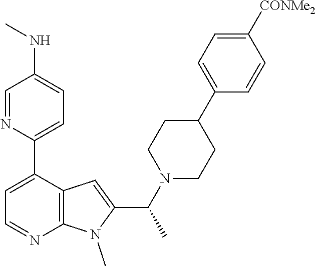 |
| 1o26 | 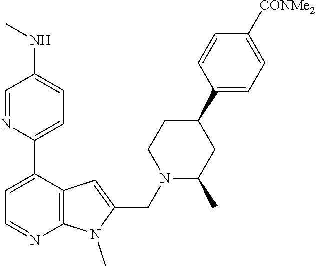 |
| 1o27 | 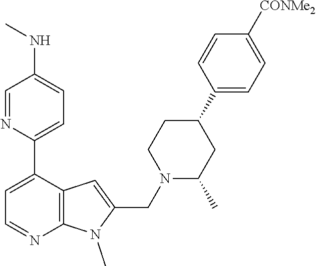 |
| 1o28 1o29 | 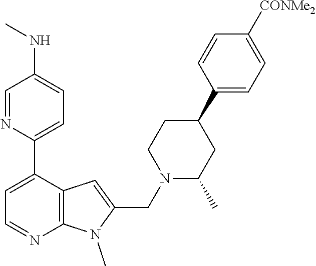 |
| | 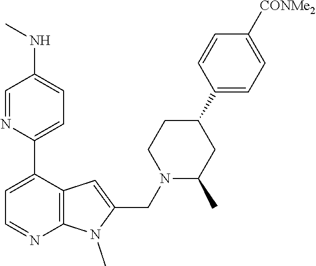 |
| 1p1 | 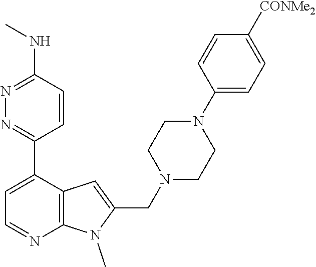 |
| 1p2 | 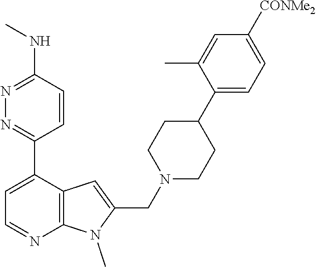 |
| 1p3 | 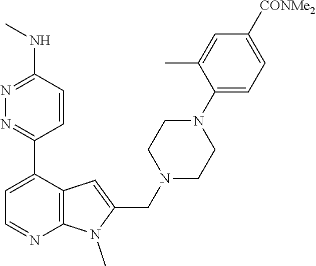 |
| 1p4 | 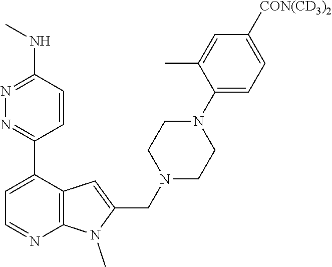 |
| 1p5 | 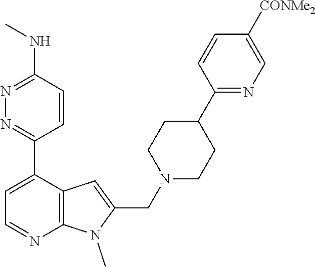 |
| 1p6 | 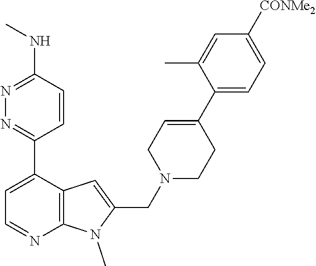 |
| 1p7 | 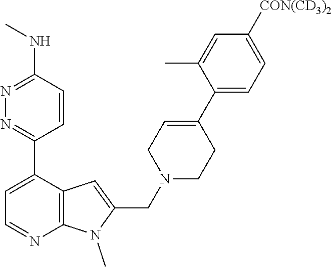 |
| 1p8 | 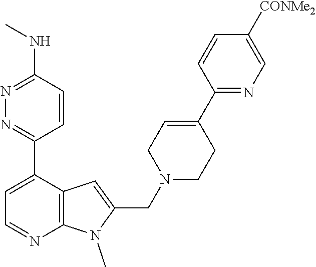 |
| 1p9 | 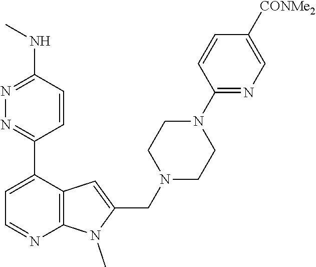 |
| 1p10 | 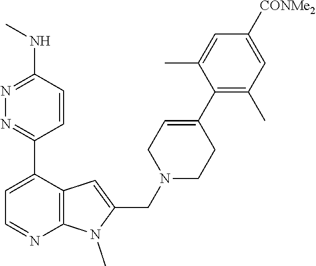 |
| 1p11 | 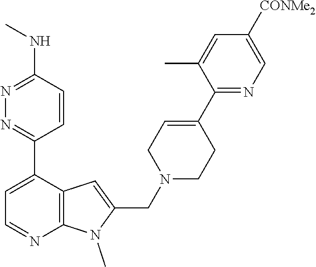 |
| 1p14 | 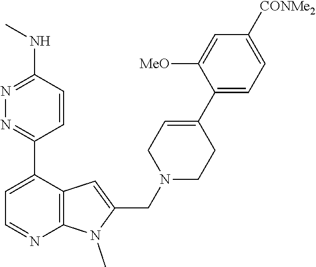 |
| 1p15 | 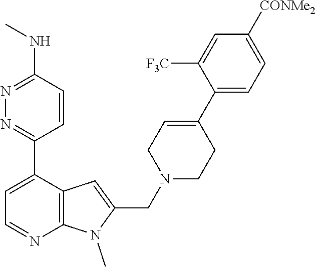 |
| 1p17 | 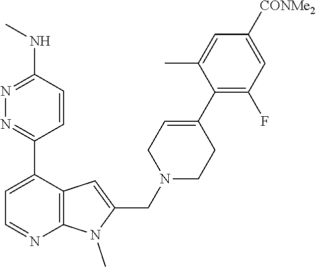 |
| 1p18 | 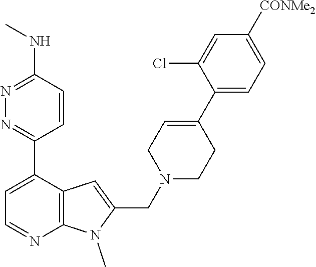 |
| 1p20 | 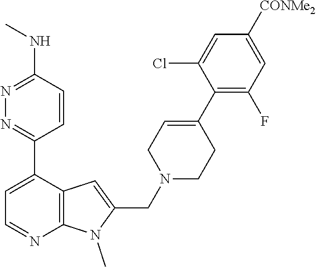 |
| 1q1 1q6 | 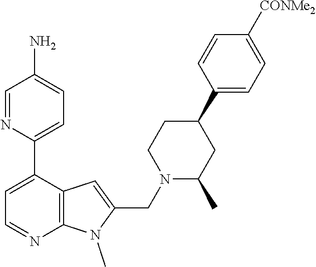 |
| | 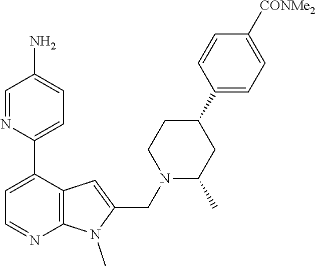 |
| 1q2 1q3 | 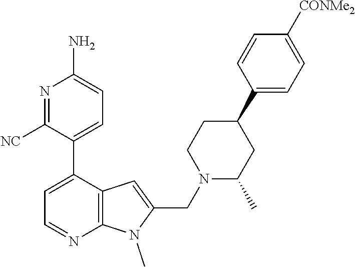 |
| | 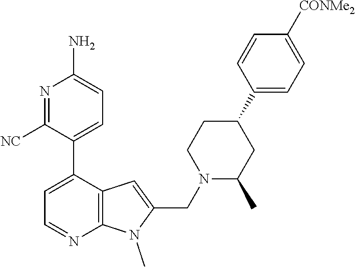 |
| 1q4 1q5 | 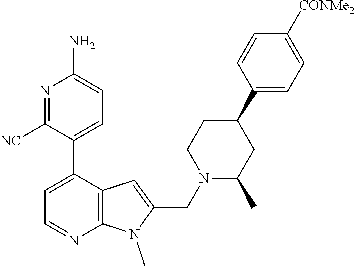 |
| | 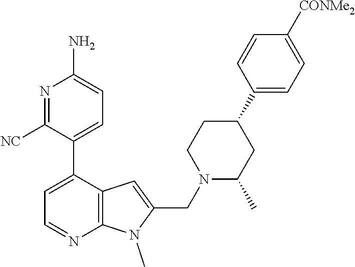 |
| 1q7 1q8 | 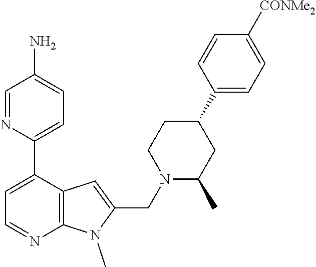 |
| | 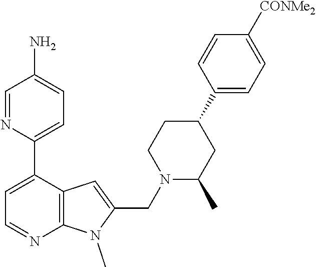 |
| 1r5 1r6 | 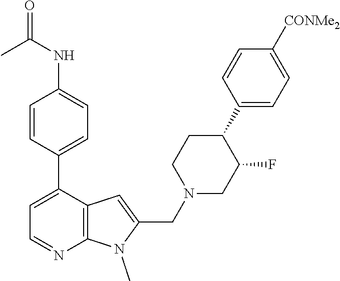 |
| | 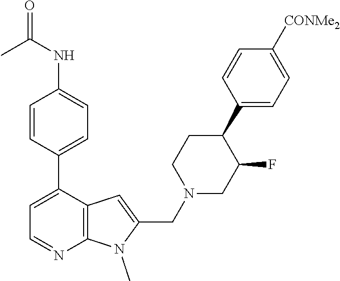 |
| 1s1 | 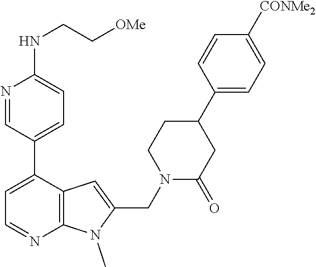 |
| 1s2 | 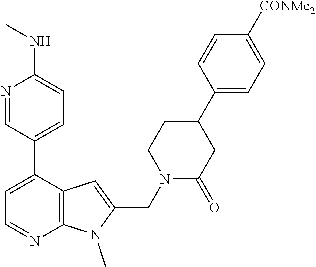 |
| 1s3 1s4 | 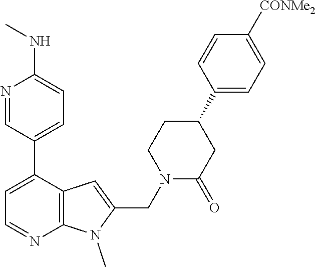 |
| | 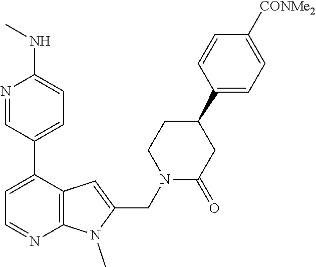 |
| 1s5 | 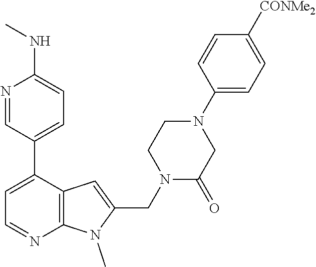 |
| 1s6 | 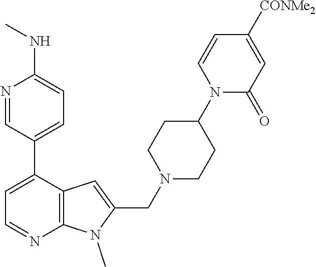 |
| 1s8 | 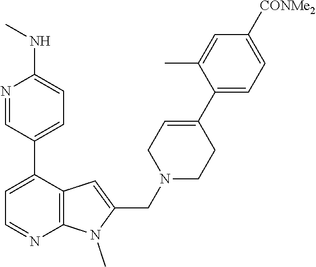 |
| 1s9 | 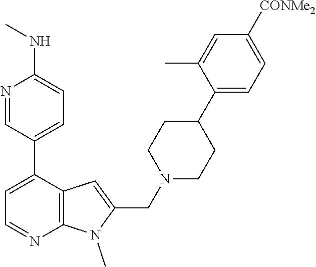 |
| 1s10 | 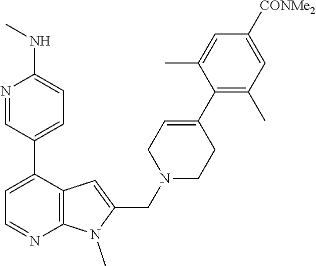 |
| 1s11 | 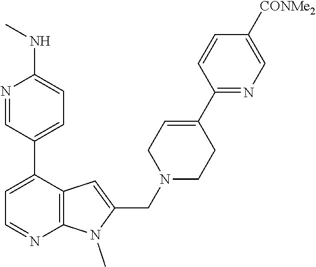 |
| 1s12 | 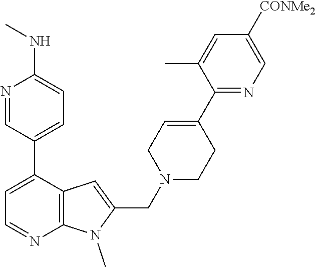 |
| 1s13 | 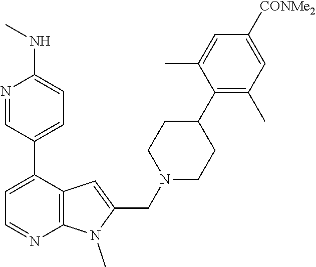 |
| 1s14 | 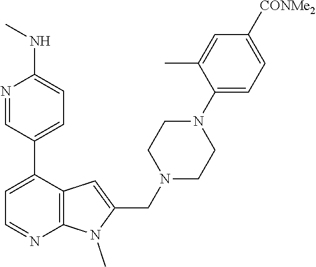 |
| 1s15 | 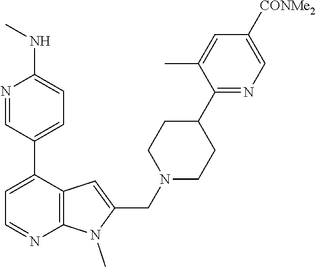 |
| 1s24 1s25 | 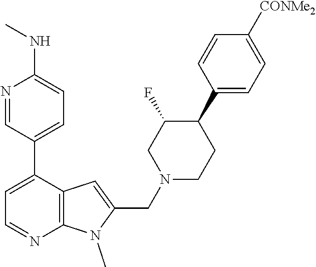 |
| | 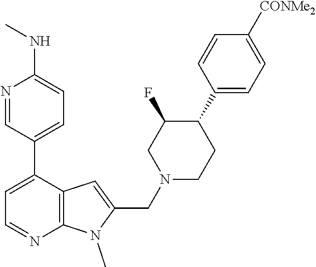 |
| 1s26 | 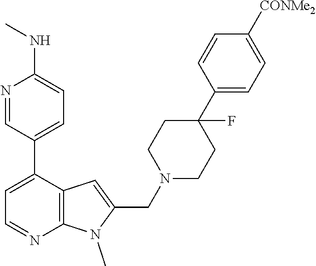 |
| 1s27 1s28 | 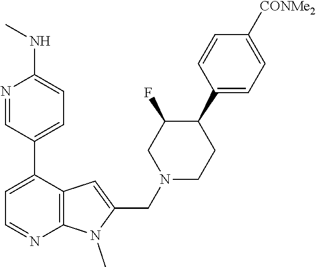 |
| | 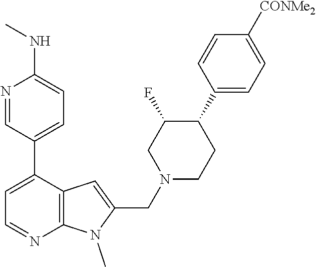 |
| 1s30 | 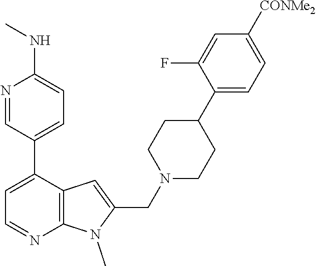 |
| 1u2 | 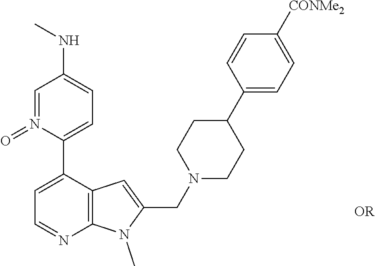 |
| | 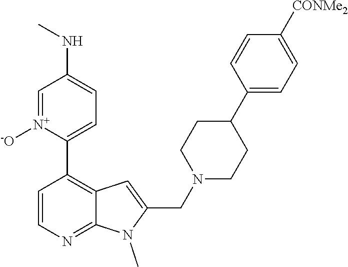 |
| 1u3 | 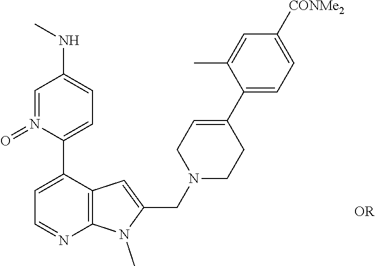 |
| | 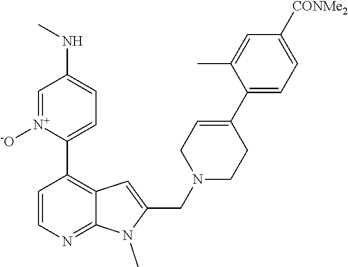 |
| 1u5 | 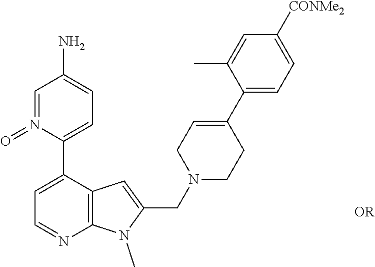 |
| | 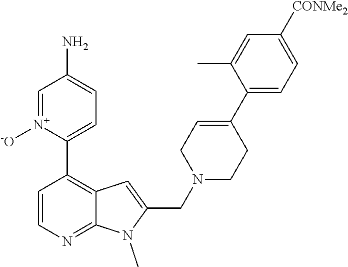 |
| 1x1 | 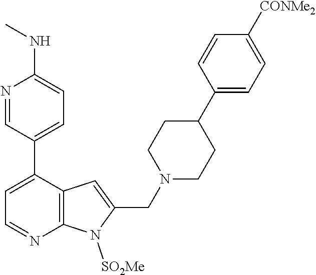 |
| 1x2 | 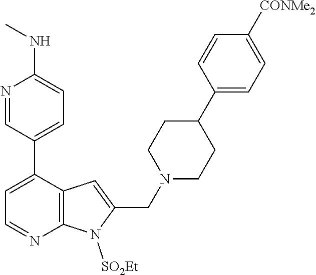 |
| 1x3 | 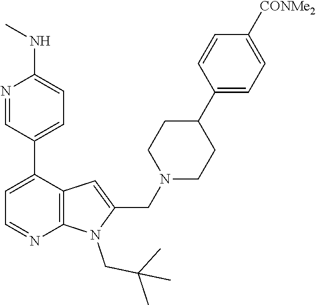 |
| 1y1 | 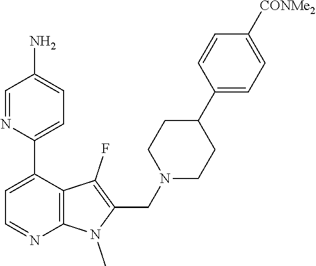 |
| 1z1 | 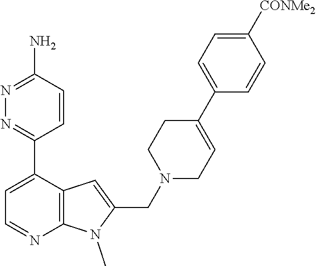 |
| 1z2 | 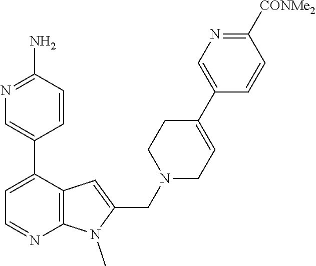 |
| 1z3 | 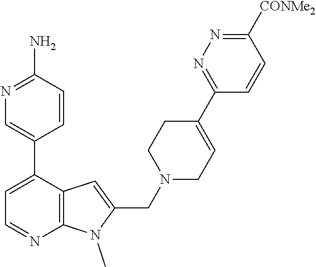 |
| 1z4 | 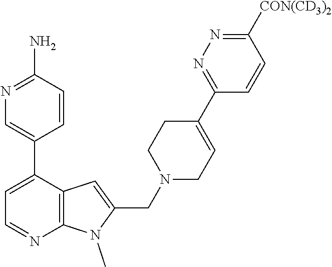 |
| 1z5 | 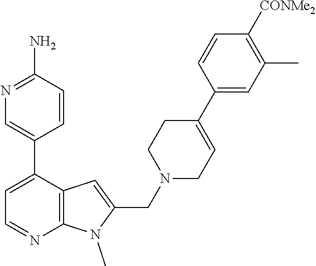 |
| 1z6 | 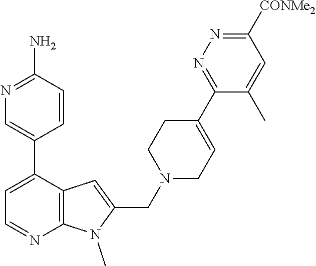 |
| 1z7 | 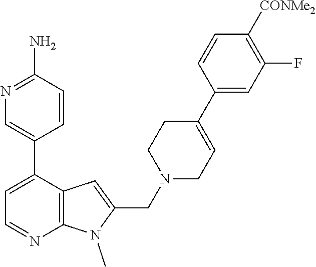 |
| 1z9 | 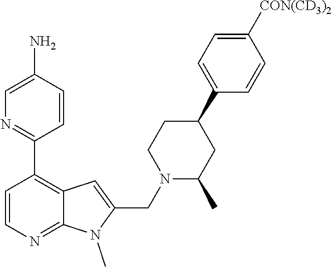 |
| 1z10 | 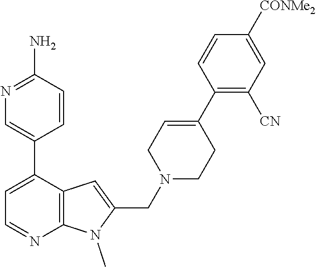 |
| 1z11 | 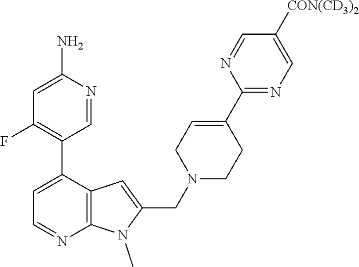 |
| 1z12 | 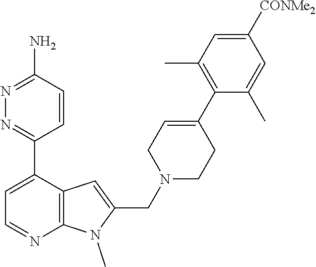 |
| 1z13 | 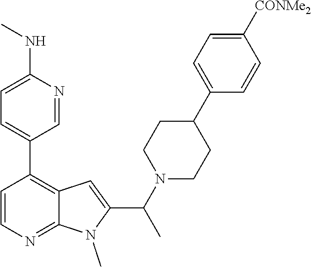 |
| 1z14 | 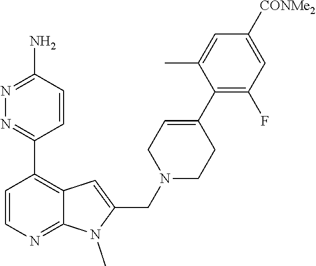 |
| 1z15 | 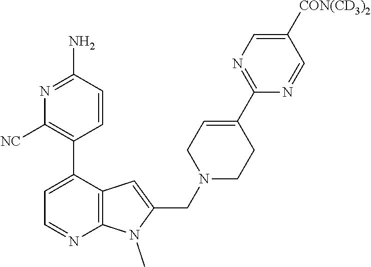 |
| 1z16 | 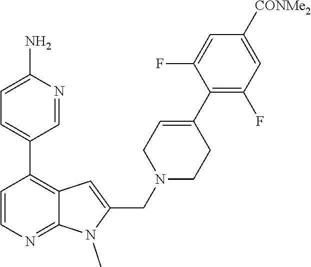 |
| 1z17 | 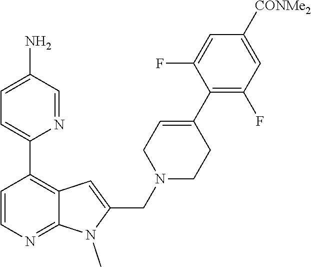 |
| 1z18 | 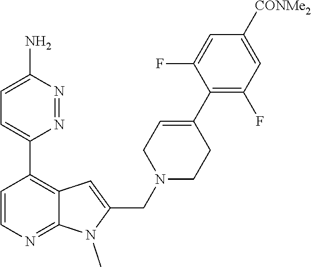 |
| 1z19 | 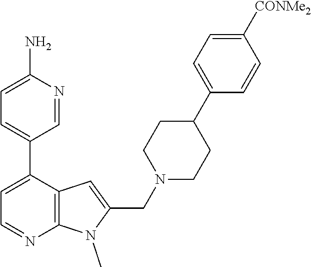 |
| 1z20 | 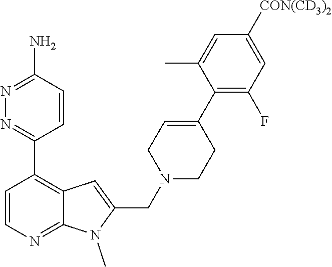 |
| 1z21 | 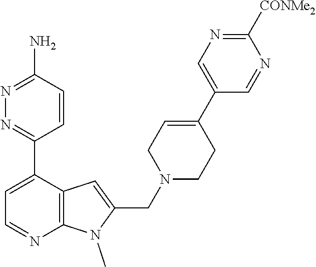 |
| 1z22 | 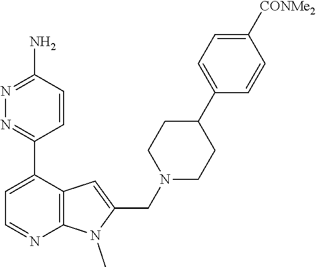 |
| 1z23 | 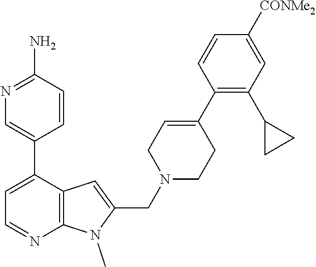 |
| 1z24 | 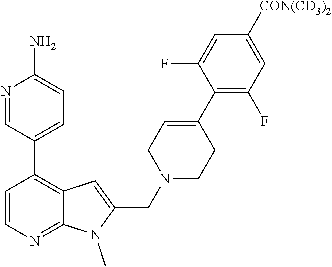 |
| 1z25 | 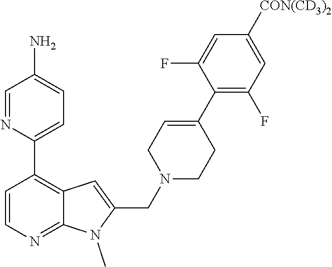 |
| 1z27 | 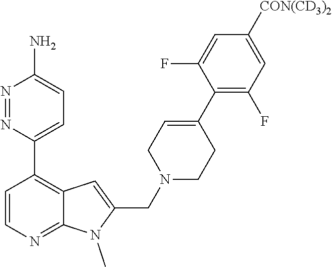 |
| 1z28 1z29 1z30 | 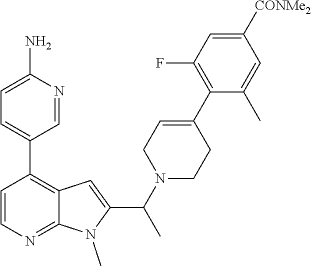 |
| | 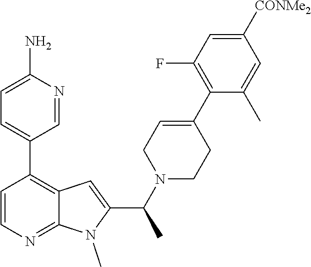 |
| | 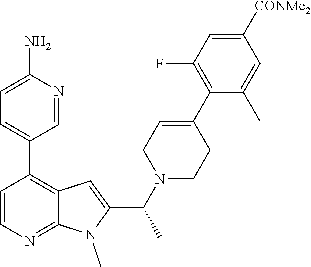 |
| 1z31 1z32 1z33 | 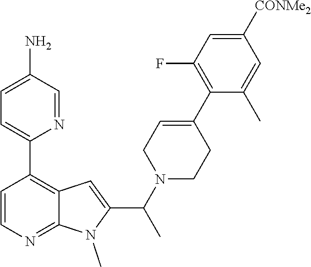 |
| | 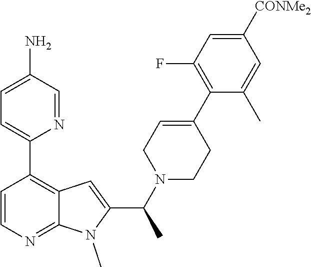 |
| | 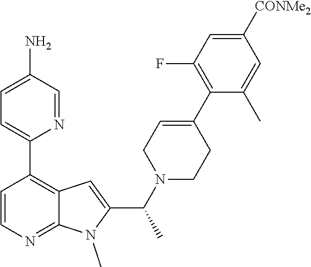 |
| 1z34 | 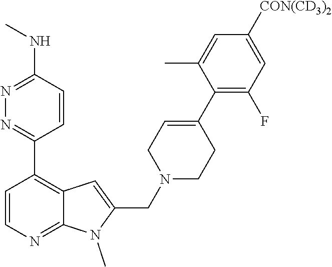 |
| 1z35 | 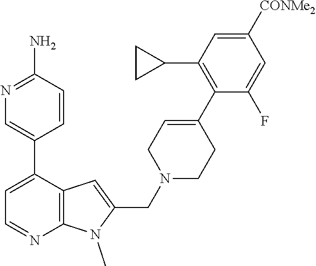 |
| 1z36 1z37 1z38 | 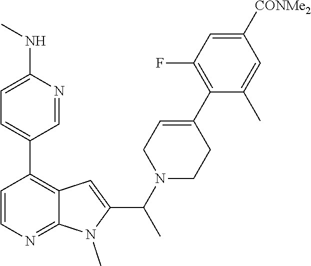 |
| | 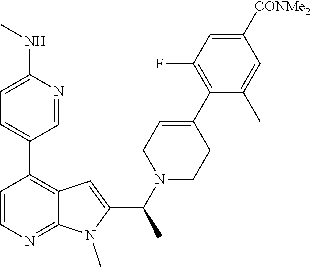 |
| | 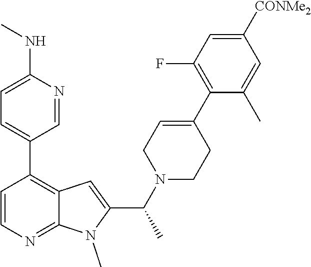 |
| 1z39 1z40 1z41 | 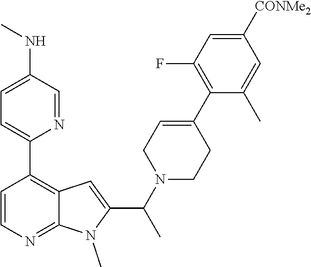 |
| | 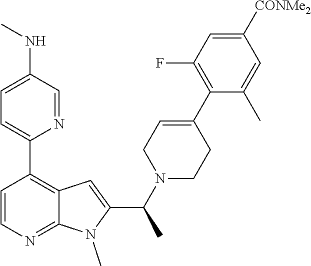 |
| | 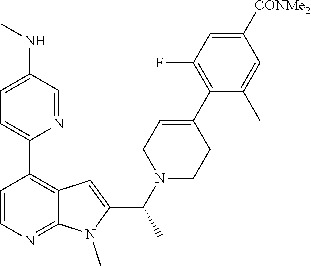 |
| 1z42 | 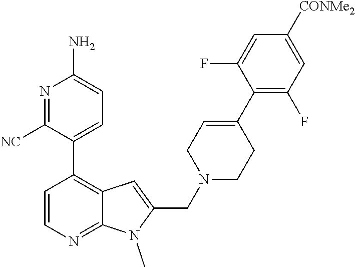 |
| 1z43 | 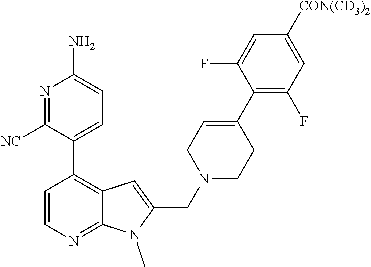 |
| 1z44 | 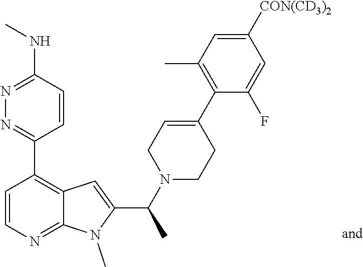 |
| 1z45 | 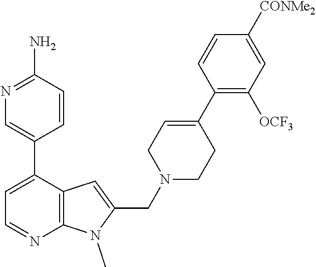 |

or a pharmaceutically acceptable salt or stereoisomer thereof.

26. A method of treating a disease, disorder or condition in a patient in need thereof, the method comprising administering to the patient a therapeutically effective amount of a compound according to claim 1, or a pharmaceutically acceptable salt or stereoisomer thereof, which disease, disorder or condition is responsive of modulation of STAT-6.

27. The method of claim 26, wherein the disease is an autoimmune disease.

28. A method of treating or ameliorating a disease in a patient in need thereof, the method comprising administering to the patient a therapeutically effective amount of a compound according to claim 1, or a pharmaceutically acceptable salt or stereoisomer thereof, wherein said disease is characterized by Th2-mediated inflammation such as atopic dermatitis, asthma, chronic rhinosinusitis with nasal polyposis, urticaria, rhinitis, eosinophilic esophagitis, food allergy, diffuse cutaneous systemic sclerosis, alopecia areata and/or COPD (chronic obstructive pulmonary disease) and different cancers such as lymphomas, triple negative breast and solid fibrous cancers either as a stand-alone treatment or in combination with other anticancer drugs such as check point inhibitors.

29.-32. (canceled)

33. A pharmaceutical composition comprising a compound according to claim 1, or a pharmaceutically acceptable salt or stereoisomer thereof, together with a pharmaceutically acceptable vehicle or excipient or pharmaceutically acceptable carrier(s).

34. A method of treating a disease or condition mediated by interleukin 4 (IL-4) or interleukin 3 (IL-3) in a patient in need thereof, the method comprising administering to the patient a therapeutically effective amount of a compound of claim 1 or a pharmaceutically acceptable salt or stereoisomer thereof.
